(19) United States
(12) Patent Application Publication
(10) Pub. No.: US 2004/0014782 A1
(43) Pub. Date: Jan. 22, 2004

(54) Combination therapy for the treatment of diseases involving inflammatory components

(76) Inventor: James E. Krause, Madison, CT (US)

Correspondence Address: Leslie-Anne Horvath, Neurogen Corporation, Patent Department, 35 NE Industrial Road, Branford, CT, 06405, US

(21) Appl. No.: 10/401,113

(22) Filed: Mar. 27, 2003

Related U.S. Application Data
(60) Provisional application No. 60/368,925, filed on Mar. 29, 2002.

(51) Int. Cl. A61K 31/4709; A61K 31/426; A61K 31/427; A61K 31/4178; A61K 31/16

(52) U.S. Cl. 514/313; 514/397; 514/365; 514/629

(57) ABSTRACT

Compositions and methods for treating diseases that are associated with inflammation are provided. Such diseases include arthritis (particularly rheumatoid arthritis) and other autoimmune disorders, asthma, cardio- and cerebrovascular disease, burns, psoriasis, reperfusion injury, and traumatic CNS and spinal cord injury. The compositions generally comprise at least one C5a antagonist and at least one C5a receptor-inactive therapeutic agent. The methods involve co-administration of at least one C5a antagonist and at least one C5a receptor-inactive therapeutic agent to a patient. The C5a antagonist and C5a receptor-inactive therapeutic agent may be present within the same composition, or may be administered separately to the patient.

### CROSS-REFERENCE TO RELATED APPLICATION

This application claims priority to U.S. Provisional Application No. 60/368,925, filed Mar. 28, 2002.

### FIELD OF THE INVENTION

This invention relates generally to compositions and methods for treating diseases that are associated with inflammation. The invention relates more specifically to compositions comprising a C5a antagonist and a C5a receptor-inactive therapeutic agent, and to therapeutic methods in which a C5a antagonist and a C5a receptor-inactive therapeutic agent are administered to a patient.

### BACKGROUND

Inflammation is a localized defense mechanism that is elicited by tissue damage or injury, and serves to destroy, dilute or wall off both injurious agents and injured tissues. Individuals suffering from inflammation typically experience redness, heat, swelling, pain and loss of function in the afflicted area. In addition, inflammatory responses cause or exacerbate the harmful effects of many diseases. Inhibition of inflammatory responses in patients afflicted with such diseases can decrease symptom severity and improve treatment outcome.

Harmful inflammation typically involves the pathogenic activation of the complement system, and in particular the C5a anaphylatoxin. C5a, a 74 amino acid peptide, is a complement component generated early in the terminal phase of the complement cascade by the proteolytic cleavage (by C5 convertase) of the complement component plasma protein C5, and is itself a plasma protein and a key mediator of inflammation. C5a promotes both vascular and cellular inflammatory responses; it has both anaphylatoxic (e.g., bronchoconstricting and vascular spasmogenic) and chemotactic effects. C5a is a potent chemoattractant for polymorphonuclear leukocytes, bringing neutrophils, basophils, eosinophils and monocytes to sites of inflammation and/or cellular injury and is one of the most potent chemotactic agents known for a wide variety of inflammatory cell types. C5a also primes neutrophils for various antibacterial functions (e.g., phagocytosis). Additionally, C5a stimulates the release of various inflammatory mediators (e.g., activated oxygen radicals, histamines, TNF-alpha, IL-1, IL-6, IL-8, prostaglandins, and leukotrienes) from various cell types and the release of lysosomal enzymes and other cytotoxic components from granulocytes. The anaphylatoxic actions of C5a result from its stimulation of smooth muscle contraction. Both the anaphylatoxic and chemotactic effects of C5a are believed to be mediated through its interaction with the C5a receptor (CD88 antigen), a 52 kD membrane-bound cell surface G-protein coupled receptor (GPCR).

A wide variety of diseases and medical procedures can result in harmful inflammation, and inhibition of C5a-mediated inflammatory responses in patients afflicted with diseases or undergoing procedures that are associated with such inflammation can be beneficial. Diseases associated with harmful inflammation include, for example, diseases of the joints, lungs, kidneys, heart, skin, liver, and digestive system, and as well as more generally, trauma and auto-immune and infectious diseases. For example, in mice, inhibition of C5a receptor activity was found to improve survival rates for sepsis (Riedemann et al. (2002) J. Clin. Invest. 110:101-108). Medical procedures associated with harmful inflammation include, for example, organ transplantation, tissue grafts, cardiopulmonary bypass and hemodialysis.

Asthma

Asthma is a lung disease characterized by a usually reversible airway obstruction, airway inflammation and increased airway responsiveness to stimuli. The airway obstruction in an asthma attack is thought to be due to the combination of bronchospasm of the smooth muscles of the bronchial tree, increased mucous section, edema of airway mucosa due to increased vascular permeability, cellular infiltration of the airway walls, and injury to airway epithelium. Studies in animal models have implicated both IgE and the complement system (and C5a in particular) in airway hyperresponsiveness and asthma pathogenesis.

Asthma may be triggered by a variety of causes such as allergic reactions, a secondary response to infections, industrial or occupational exposures, ingestion of certain chemicals or drugs, exercise, and vasculitis. Regardless of the trigger, many of the pathological features of asthma can be attributed to mast cell degranulation. Such responses are, at least in part, mediated by IgE antibodies, which trigger mast cell degranulation in the lung interstitium. The mast cell degranulation releases, among other factors, histamine, bradykinin, and slow-reacting substance of anaphylaxis (SRS-A) which includes the leukotrienes C, D and E, prostaglandins including PGF₂, PGF_{2α} and PGD₂, and thromboxane A₂. The histamine then attaches to receptor sites in the larger bronchi, causing irritation, inflammation and edema. The SRS-A attaches to receptor sites in the smaller bronchi, causing edema and attracting prostaglandins, which enhance the effects of histamine in the lungs. With the help of the prostaglandins, histamine also stimulates excessive mucous secretion, further narrowing the bronchial lumen. When the asthmatic inhales, the narrowed bronchial lumen still expands slightly, allowing air to reach the alveoli. However, upon exertion to exhale, the increased thoracic pressure closes the bronchial lumen completely. Thus, in an asthma attack, air can enter, but not exit the lungs. Mucous then fills the lung bases, inhibiting alveolar ventilation. In an effort to compensate for lowered alveolar ventilation, blood is shunted to other alveoli. Without adequate compensation, hypoxia, and in extreme cases, respiratory acidosis may result.

In many cases, there are two phases to an allergic asthma attack, an early phase and a late phase which follows 4-6 hours after bronchial stimulation. The early phase includes the immediate inflammatory response including the reactions caused by the release of cellular mediators from mast cells. Late phase reactions develop over a period of hours and are characterized histologically by an early influx of polymorphonuclear leukocytes and fibrin deposition followed later by infiltration of eosinophils. Late phase reactions increase airway reactivity and lead to prolonged asthmatic exacerbations that may last from hours to days to months in some subjects.

Asthma is most commonly treated with oral and inhaled bronchodilators. Such agents alleviate the symptoms of asthma, but have no effect on the underlying inflammation. Corticosteroids are also used to treat the inflammation, but these drugs can have serious side effects and many patients continue to suffer from incompletely controlled asthma.

Skin Disorders Associated with Inflammation

The complement system is an important skin defense mechanism, and complement activation (particularly C5a) is involved in the pathogenesis of a variety of skin conditions such as bullous pemphigoid, lichen planas, herpes gestationis and psoriasis.

Psoriasis is one of the most common dermatologic diseases, affecting about 2 percent of the population. This condition presents as elevated lesions that vary in size from one to several centimeters, and results from an overproduction of epidermal cells. The increased production of epidermal cells is due to a shortened cell cycle time, an increase in the absolute number of cells capable of proliferating and an increased rate of division. The thickened epidermis, overgrowth of blood vessels, and infiltration of neutrophils and lymphocytes account for the psoriatic lesions being raised and easily palpable. T cell mediated immune responses appear to be responsible for the inflammation and hyperproliferation of epidermal cells. Neutrophils are found in psoriatic lesions, associated with increased levels of plasminogen activator. Psoriatic fibroblasts have increased levels of enzymes involved in collagen synthesis, secondary to expansion of the papillary dermis. Psoriatic plaques comprise HLA-DR positive keratinocytes and Langerhans cells, as well as activated T cells expressing elevated levels of IL-2 receptors and secreting cytokines including TNF and interleukin-6, which stimulate skin cell growth.

It is not known what causes psoriasis, although there is evidence of a genetic predisposition and an autoimmune etiology. Onset may be triggered by systemic infections such as strep throat infection, skin injury, vaccinations, and certain oral medications such as steroids, which induce inflammation and excessive skin cell reproduction. Psoriasis can be exacerbated by additional factors such as stress and diet. Regardless of the trigger, C5a activation appears to play a direct role in the pathogenesis of the disease.

Rheumatoid Arthritis

Rheumatoid arthritis (RA) is a chronic disease characterized by persistent joint inflammation (inflammatory synovitis). Early clinical manifestations of the disease include pain, swelling and tenderness of the joints that is initially poorly localized. Many patients exhibit general fatigue, weakness, loss of appetite, low-grade fever and musculoskeletal symptoms before joint pain becomes localized. As the disease progresses, joint pain, swelling and stiffness become more evident. Movement, particularly after periods of inactivity, becomes painful and difficult. The persistent inflammation caused by rheumatoid arthritis often leads to destruction or weakening of ligaments and tendons, and destruction of cartilage and bone. Deformities of the hands and feet, due to fibrous or bony ankylosis or soft tissue contracture, are often present in advanced disease. Current treatments of RA can be divided into two types—therapies which act to alleviate the symptoms of the disease, such as pain medications, and disease-modifying therapies which act on some underlying cause of the disease and slow its progression, such as steroids. Inhibitors of cyclooxygenase (COX) enzymes, which inhibit prostaglandin production and thereby reduce inflammation are commonly used to treat rheumatoid arthritis. Such compounds may inhibit COX enzymes non-specifically (e.g., the salicylates), or may specifically inhibit COX-2. Injections of gold sodium thiomalate and oral administration of gold compounds have also been shown to suppress the synovitis of active rheumatoid arthritis. In some case, surgery may be performed.

Considerable experimental evidence has demonstrated the presence of increased levels of C5a in rheumatoid arthritis. Antibodies that bind to C5 and inhibit the conversion of C5 to C5a (and C5b) by C5 convertase have been shown to be effective in reducing symptoms of rheumatoid arthritis in animal models of arthritis. A C5a receptor antagonist also showed activity as an anti-arthritic agent in a rat RA model (Woodruff et al. (2002) Arthritis Rheum 46:2476-85).

Other Autoimmune Disorders

A number of other autoimmune disorders and pathologic autoimmune responses are known to have an inflammatory component, such as multiple sclerosis, myasthenia gravis, Alzheimer's disease, glomerulonephritis, Crohn's disease, lupus erythematosus and irritable bowel syndrome. Considerable experimental evidence has demonstrated the presence of increased levels of C5a in a number of autoimmune diseases and inflammatory disorders. In addition, anti-C5 antibodies also been used to treat glomerulonephritis, a disease characterized by inflammation of the kidney (see U.S. Pat. No. 6,355,245).

Medical Procedures

A variety of medical procedures involve the introduction of foreign matter into the patient's body. Such procedures generally trigger, and are complicated by, inflammation. For example, inflammation may result from cardiopulmonary bypass surgery or hemodialysis. Rejection of transplanted organs and tissue grafts also has an inflammatory component. In some cases, for example, the blood supply to the transplant becomes blocked due to inflammation of the blood vessels leading to the transplanted organ. Current therapy for transplant rejection involves a regimen of immunosuppressants, including cyclosporin A, tacrolimus, and rapamycin (sirolimus). However, patients continue to have a 20 to 50 percent risk of rejecting a donated organ during the first three years following transplantation, and less than 50 percent of patients have functioning transplants after 10 years. Additionally, chronic use of immunosuppressants can lead to impairment of the recipients' immune system.

Cardio- and Cerebrovascular Disease

Low-level inflammation has been correlated to the incidence of heart attack and stroke. C-reactive protein, a marker for inflammation, has been shown to be associated with increased risk for cardiovascular events (Ridker et al. (2002) N. Engl. J. Med. 347:1557-1565). In a study of men with levels of C-reactive protein considered to be within the normal range, the benefits of aspirin in the prevention of heart attack and stroke were more pronounced in individuals with higher levels of C-reactive protein, suggesting that aspirin's anti-inflammatory effects are responsible for the reduction in heart attacks and strokes (Ridker et al (1997) N. Eng. J. Med. 336:973079).

Ischemia-Reperfusion Injury

Ischemia is a condition in which blood flow (and thus oxygen) is restricted to a part of the body and may occur, for example, due to thrombosis or surgery. Reperfusion injury occurs when a blood flow is restored to a blood vessel that has been previously occluded. Reperfusion injury has also been found to occur during surgeries in which blood vessels are not occluded but in which a heart bypass pump is employed. The hypoxic conditions in occluded blood vessels induces the production of a number of pro-inflammatory cytokines. While prompt restoration of blood flow is necessary to restore normal function, reperfusion also causes the destruction of additional cells and an intense inflammatory reaction that involves C5a activation. The pro-inflammatory cytokines produced while the vessel was occluded causes leads leukocyte recruitment and subsequent destruction of the endothelium. Additional damage may occur due to obstruction of microcapillaries by leukocytes. In mice, inhibition of C5a receptor activity has been found to improve survival rates for ischemia-reperfusion injury (De Vries et al. (2003) Transplantation 75:375-82). A small molecule C5a receptor antagonist also was shown to protect kidneys from ischemia-reperfusion injury in rats (Arumugam et al. (2003) Kidney Int. 63:134-42).

While some treatments are presently available for both inflammatory and non-inflammatory disease components, there remains a need in the art for improved anti-inflammatory medications and methods for using such improved medications in combination with currently available therapies. The present invention fulfills this need and provides further related advantages.

### SUMMARY OF THE INVENTION

In a first aspect, the invention provides compositions useful for the treatment of diseases with inflammatory components, such as arthritis (particularly rheumatoid arthritis) and other autoimmune disorders, asthma, cardio- and cerebrovascular disease, psoriasis, reperfusion injury, and traumatic CNS and spinal cord injury. Such compositions comprise at least one C5a receptor antagonist (hereinafter a “C5a antagonist”) and at least one C5a receptor-inactive therapeutic agent (i.e., a therapeutic agent that is not a C5a antagonist).

Preferred properties for C5a antagonists for use in the practice of the invention are one or preferably 2 or most preferably all 3 of: 1) having a molecular mass less than 700 a.m.u. 2) being nonpeptidic (i.e., do not contain amino acids joined by a peptide bond; preferably do not contain any amino acid moiety) and 3) having minimal agonist activity (i.e., induce an increase in the basal activity of the C5a receptor in the absence of C5a that is less than 5% of the increase that would be induced by C5a, preferably inducing no statistically significant increase). Preferred C5a antagonists for used in the practice of the invention include neutral antagonists and inverse agonists of the C5a receptor.

Within certain embodiments, the C5a receptor-inactive therapeutic agent is an NSAID, a cyclo-oxygenase enzyme inhibitor, a gold compound, a salicylate, a steroid such as a corticosteroid, methotrexate, lefunomide, a TNF antagonist, a cholesterol lowering agent, an HMG-CoA reductase inhibitor, a platelet aggregation inhibitor, or an anti-hypertensive agent.

Within further aspects, the present invention provides pharmaceutical compositions, comprising a C5a antagonist in combination with a C5a receptor-inactive therapeutic agent and a pharmaceutically acceptable carrier or excipient. Pharmaceutical formulations, such as tablets, pills and capsules, containing a C5a antagonist and a C5a receptor-inactive therapeutic agent are included in the invention. Pharmaceutical formulations of the invention may include additional active or inert ingredients. Processes for preparing such pharmaceutical compositions and pharmaceutical formulations are included in the invention.

Also provided are packages comprising such a pharmaceutical composition and instructions for use to treat a patient suffering from arthritis or another autoimmune disorder, asthma, cardio- and cerebrovascular disease, psoriasis, reperfusion injury, or traumatic CNS or spinal cord injury. The C5a antagonist and a C5a receptor-inactive therapeutic agent may be provided each in a separate container within the package or—where both are to be given by the same route of administration—preferably combined in a single formulation.

Methods are further provided, within other aspects for treating a patient suffering from arthritis or another autoimmune disorder, asthma, cardio- or cerebrovascular disease, psoriasis, reperfusion injury, burns, or traumatic CNS or spinal cord injury, comprising administering to the patient a therapeutically effective amount of a C5a receptor antagonist in combination with a therapeutically effective amount of a C5a receptor-inactive therapeutic agent. The combination therapy provided herein encompasses either or both of 1) the administration of a C5a antagonist and a C5a receptor-inactive therapeutic agent together, preferably in a single pharmaceutical formulation, and 2) the administration of a C5a antagonist in a first formulation and the separate administration of a C5a receptor-inactive therapeutic agent in a second pharmaceutical formulation.

These and other aspects of the present invention will become apparent upon reference to the following detailed description.

### DETAILED DESCRIPTION OF THE INVENTION

Terminology

A “C5a antagonist” or “C5a receptor antagonist” is any compound that exhibits C5a antagonist activity within the a C5a receptor-mediated chemotaxis, radioligand binding assay, or calcium mobilization assay as provided herein. In other words, in a calcium mobilization assay, a compound is a C5a antagonist if incubation of cells with 1 uM of C5a antagonist results in at least a 2-fold increase in the fluorescence response relative to that measured in the presence of C5a alone. In a chemotaxis assay, a compound is a C5a antagonist if it displays an affinity constant or IC₅₀ of 1 uM or less. Preferably, a C5a antagonist displays an IC₅₀ of less than 500 nM, 200 nM, 100 nM, 50 nM, 25 nM, 10 nM or 5 nM (in a chemotaxis and/or calcium mobilization assay) in a standard C5a receptor-mediated chemotaxis assay, radioligand binding assay, or calcium mobilization assay. In certain embodiments, C5a antagonists provided herein inhibit activation and/or activity of a primate C5a receptor, such as human C5a receptor, which may be a cloned, recombinantly expressed receptor or a naturally expressed receptor. For treating non-human animals of any particular species, a compound exhibiting high affinity for the C5a receptor of that particular species is preferred.

As used herein, “therapeutic agent” refers to a compound which has been shown to exhibit clinical efficacy in reducing the symptoms of one or more of arthritis (preferably rheumatoid arthritis) or another autoimmune disorder, asthma, cardio- or cerebrovascular disease, psoriasis, reperfusion injury, burns, or traumatic CNS or spinal cord injury. A “C5a receptor-inactive therapeutic agent” is such an agent that does not satisfy the criteria (above) for a C5a antagonist.

As used herein, “active agent” refers to either or both of the C5a antagonist and the C5a receptor-inactive therapeutic agent. This term is intended to encompass all salt, ester and prodrug forms of C5a antagonists and C5a receptor-inactive therapeutic agents, even where the prodrug is not active itself but is converted to the active form after administration to the patient.

An active agent is said to be “administered” if it is caused to be contacted with a patient so as to provide a detectable therapeutic effect. Administration may be oral, intranasal, topical, rectal or parenteral. The term parenteral as used herein includes subcutaneous, intradermal, intravascular (e.g., intravenous), intramuscular, spinal, intracranial, intrathecal and intraperitoneal injection, as well as any similar injection or infusion technique.

A “condition with a pathogenic inflammatory component” is any disease, disorder or injury that is caused, prolonged or exacerbated by C5a-mediated inflammation. Such conditions include, but are not limited to, arthritis (such as rheumatoid arthritis) and other autoimmune disorders (e.g., multiple sclerosis, myasthenia gravis, Alzheimer's disease, glomerulonephritis, Crohn's disease, Guillain-Barre Syndrome, lupus erythematosus and irritable bowel syndrome); asthma and other lung disorders, including respiratory distress syndrome; skin conditions and injuries such as psoriasis, bullous pemphigoid, lichen planas, burns and wounds; cardio- and cerebrovascular disease, including restenosis; ischemia-reperfusion injury; trauma (e.g., CNS); sepsis and other infections; hemorrhagic shock; and multiple organ system failure. Such conditions also include medical procedures such as organ transplantation (e.g., lung), tissue grafts, hemodialysis, and cardiopulmonary bypass surgery, where recovery may be inhibited or delayed as a result of inflammation.

C5a antagonists used in the compositions and methods provided herein are generally described using standard nomenclature. Certain compounds described herein contain one or more asymmetric elements such as stereogenic centers, stereogenic axes and the like (e.g., asymmetric carbon atoms) so that the compounds can exist in different stereoisomeric forms. These compounds can be, for example, racemates or optically active forms. For compounds with two or more asymmetric elements, these compounds can additionally be mixtures of diastereomers. Unless otherwise specified all optical isomers and mixtures thereof are encompassed for compounds having asymmetric centers. In addition, compounds with carbon-carbon double bonds may occur in Z- and E-forms, with all isomeric forms of the compounds being included in the present invention unless otherwise specified. Where a compound exists in various tautomeric forms, the invention is not limited to any one of the specific tautomers, but rather encompasses all tautomeric forms.

The present invention is intended to include all isotopes of atoms occurring in the present compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

Certain compounds are described herein using a general formula, such as Formula I, that includes variables, such as various R groups, Ar¹, Ar², and x. Unless otherwise specified, each variable within such a formula is defined independently of other variables. Thus, for example, if a group is shown to be substituted with 0-2 R*, then said group may optionally be substituted with up to two R* groups and R* at each occurrence is selected independently from the definition of R*. Also, combinations of substituents and/or variables are permissible only if such combinations result in stable compounds.

A “substituent,” as used herein, refers to a molecular moiety that is covalently bonded to an atom within a molecule of interest. For example, a “ring substituent” may be a moiety such as a halogen, alkyl group, haloalkyl group or other substituent discussed herein that is covalently bonded to an atom (preferably a carbon or nitrogen atom) that is a ring member. The term “substituted,” as used herein, means that any one or more hydrogens on the designated atom is replaced with a selection from the indicated substituents, provided that the designated atom's normal valence is not exceeded, and that the substitution results in a stable compound (i.e., a compound that can be isolated, characterized and tested for biological activity). When a substituent is oxo (i.e., =0), then 2 hydrogens on the atom are replaced. When aromatic moieties are substituted by an oxo group, the aromatic ring is replaced by the corresponding partially unsaturated ring. For example a pyridyl group substituted by oxo is a tetrahydropyridone.

The phrase “optionally substituted” indicates that a group may either be unsubstituted or substituted at one or more of any of the available positions, typically 1, 2, 3, 4, or 5 positions, by one or more suitable substituents such as those disclosed herein. Various groups within the compounds and formulae set forth herein are “optionally substituted” including, for example, R¹, R², and Ar¹. Optional substitution may also be indicated by the phrase “substituted with from 0 to X substituents,” in which X is the maximum number of substituents.

Suitable substituents include, for example, halogen, cyano, amino, hydroxy, nitro, azido, carboxamido, —COOH, SO₂NH₂, alkyl (e.g., C₁-C₈alkyl), alkenyl (e.g., C₂-C₈alkenyl), alkynyl (e.g., C₂-C₈alkynyl), alkoxy (e.g., C₁-C₈alkoxy), alkyl ether (e.g., C₂-C₈alkyl ether), alkylthio (e.g., C₁-C₈alkylthio), mono- or di-(C₁-C₈alkyl)amino, haloalkyl (e.g., C₁-C₆haloalkyl), hydroxyalkyl (e.g., C₁-C₆hydroxyalkyl), aminoalkyl (e.g., C₁-C₆aminoalkyl), haloalkoxy (e.g., C₁-C₆haloalkoxy), alkanoyl (e.g., C₁-C₈alkanoyl), alkanone (e.g., C₁-C₈alkanone), alkanoyloxy (e.g., C₁-C₈alkanoyloxy), alkoxycarbonyl (e.g., C₁-C₈alkoxycarbonyl), mono- and di-(C₁-C₈alkyl)amino, mono- and di-(C₁-C₈alkyl)aminoC₁-C₈alkyl, mono- and di-(C₁-C₈alkyl)carboxamido, mono- and di-(C₁-C₈alkyl)sulfonamido, alkylsulfinyl (e.g., C₁-C₈alkylsulfinyl), alkylsulfonyl (e.g., C₁-C₈alkylsulfonyl), aryl (e.g., phenyl), arylalkyl (e.g., (C₆-C₁₈aryl)C₁-C₈alkyl, such as benzyl and phenethyl), aryloxy (e.g., C₆-C₁₈aryloxy such as phenoxy), arylalkoxy (e.g., (C₆-C₁₈aryl)C₁-C₈alkoxy) and/or 3- to 8-membered heterocyclic groups. Certain groups within the formulas provided herein are optionally substituted with from 1 to 3, 1 to 4 or 1 to 5 independently selected substituents.

A dash (“—”) that is not between two letters or symbols is used to indicate a point of attachment for a substituent. For example, —CONH₂ is attached through the carbon atom.

As used herein, “alkyl” is intended to include both branched and straight-chain saturated aliphatic hydrocarbon groups, and where specified, having the specified number of carbon atoms. Thus, the term C₁-C₆alkyl, as used herein, indicates an alkyl group having from 1 to 6 carbon atoms. “C₀-C₄alkyl” refers to a bond or a C₁-C₄alkyl group. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, n-propyl, isopropyl, n-butyl, sec-butyl, tert-butyl, pentyl, 2-pentyl, isopentyl, neopentyl, hexyl, 2-hexyl, 3-hexyl, and 3-methylpentyl. “Aminoalkyl” is an alkyl group as defined herein substituted with one or more —NH₂ groups. “Hydroxyalkyl” is a hydroxy group as defined herein substituted with one or more —OH groups.

“Alkenyl” refers to a straight or branched hydrocarbon chain comprising one or more unsaturated carbon-carbon bonds, such as ethenyl and propenyl. Alkenyl groups include C₂-C₈alkenyl, C₂-C₆alkenyl and C₂-C₄alkenyl groups (which have from 2 to 8, 2 to 6 or 2 to 4 carbon atoms, respectively), such as ethenyl, allyl or isopropenyl.

“Alkynyl” refers to straight or branched hydrocarbon chains comprising one or more triple carbon-carbon bonds. Alkynyl groups include C₂-C₈alkynyl, C₂-C₆alkynyl and C₂-C₄alkynyl groups, which have from 2 to 8, 2 to 6 or 2 to 4 carbon atoms, respectively. Alkynyl groups include for example groups such as ethynyl and propynyl.

“Alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

The term “alkanoyl” refers to an acyl group in a linear or branched arrangement (e.g., —(C═O)-alkyl). Alkanoyl groups include C₂-C₈alkanoyl, C₂-C₆alkanoyl and C₂-C₄alkanoyl groups, which have from 2 to 8, 2 to 6, or 2 to 4 carbon atoms, respectively. “Cialkanoyl” refers to —(C═O)—H, which (along with C₂-C₈alkanoyl) is encompassed by the term “C₁-C₈alkanoyl.”

The term, “alkyl ether” refers to a linear or branched ether substituent linked via a carbon-carbon bond. Alkyl ether groups include C₂-C₈alkyl ether, C₂-C₆alkyl ether and C₂-C₆alkyl ether groups, which have 2 to 8, 2 to 6, or 2 to 4 carbon atoms, respectively. By way of example, a C₂alkyl ether group has the structure —CH₂—O—CH₃.

The term “alkoxycarbonyl” refers to an alkoxy group linked via a carbonyl (i.e., a group having the general structure —C(═O)—O-alkyl). Alkoxycarbonyl groups include C₂-C₈, C₂-C₆, and C₂-C₄alkoxycarbonyl groups, which have from 2 to 8, 2 to 6, or 2 to 4 carbon atoms, respectively. “C₁alkoxycarbonyl” refers to —C(═O)OH, and is encompassed by “C₁-C₈alkoxycarbonyl.”

“Alkanoyloxy,” as used herein, refers to an alkanoyl group linked via an oxygen bridge (i.e., a group having the general structure —O—C(═O)-alkyl). Alkanoyloxy groups include C₂-C₈, C₂-C₆, and C₂-C₄alkanoyloxy groups, which have from 2 to 8, 2 to 6, or 2 to 4 carbon atoms, respectively.

As used herein, the term “alkylthio” refers to an alkyl group attached via a thioether linkage. Alkylthio groups include C₁-C₈alkylthio, C₁-C₆alkylthio and C₁-C₄alkylthio, which have from 1 to 8, 1 to 6 or 1 to 4 carbon atoms, respectively.

“Alkylsulfinyl,” as used herein, refers to an alkyl group attached via a sulfinyl linkage. Alkylsulfinyl groups include C₁-C₈alkylsulfinyl, C₁-C₆alkylsulfinyl, and C₁-C₄alkylsulfinyl, which have from 1 to 8, 1 to 6, and 1 to 4 carbon atoms, respectively.

By “alkylsulfonyl,” as used herein, is meant an alkyl group attached via a sulfonyl linkage. Alkylsulfonyl groups include C₁-C₈alkylsulfonyl, C₁-C₆alkylsulfonyl, and C₁-C₄alkylsulfonyl, which have from 1 to 8, 1 to 6, and 1 to 4 carbon atoms, respectively.

“Alkylamino” refers to a secondary or tertiary amine having the general structure —NH-alkyl or —N(alkyl)(alkyl), wherein each alkyl may be the same or different. Such groups include, for example, mono- and di-(C₁-C₈alkyl)amino groups, in which each alkyl may be the same or different and may contain from 1 to 8 carbon atoms, as well as mono- and di-(C₁-C₆alkyl)amino groups and mono- and di-(C₁-C₄alkyl)amino groups. Alkylaminoalkyl refers to an alkylamino group linked via an alkyl group (i.e., a group having the general structure -alkyl-NH-alkyl or -alkyl-N(alkyl)(alkyl)). Such groups include, for example, mono- and di-(C₁-C₈alkyl)aminoC₁-C₈alkyl, mono- and di-(C₁-C₆alkyl)aminoC₁-C₆alkyl, and mono- and di-(C₁-C₄alkyl)aminoC₁-C₄alkyl, in which each alkyl may be the same or different.

The term “carboxamido” or “amido” refers to an amide group (i.e., —(C═O)NH₂). “Alkylcarboxamido” refers to —NHC(═O)alkyl, preferably —NHC(═O)C₁-C₂alkyl.

The term “cycloalkyl” refers to hydrocarbon ring groups, having the specified number of carbon atoms, usually from 3 to about 8 ring carbon atoms, or from. Cycloalkyl groups include C₃-C₈, and C₃-C₇ cycloalkyl groups, which have from 3 to 8 and 3 to 7 carbon atoms, respectively. Examples of cycloalkyl groups include cyclopropyl, cyclobutyl, cyclopentyl, and cyclohexyl groups, as well as bridged and caged saturated ring groups such as norbornane or adamantane and the like.

In the term “(cycloalkyl)alkyl,” “cycloalkyl” and “alkyl” are as defined above, and the point of attachment is on the alkyl group. This term encompasses, but is not limited to, cyclopropylmethyl, cyclohexylmethyl, and cyclohexylethyl.

The term “halogen” indicates fluorine, chlorine, bromine, or iodine.

“Haloalkyl” refers to both branched and straight-chain saturated aliphatic hydrocarbon groups having the specified number of carbon atoms, substituted with I or more halogen atoms. Examples of haloalkyl include, but are not limited to, trifluoromethyl, difluoromethyl, 2-fluoroethyl, and penta-fluoroethyl.

“Haloalkoxy” indicates a haloalkyl group as defined above attached through an oxygen bridge.

As used herein, the term “aryl” indicates aromatic groups containing only carbon in the aromatic ring(s). Such aromatic groups may be further substituted with carbon or non-carbon atoms or groups. Typical aryl groups contain 1 to 3 separate or fused rings, at least one of which is aromatic, and from 6 to about 18 ring atoms, without heteroatoms as ring members. Specifically preferred carbocyclic aryl groups include phenyl and napthyl, including 1-naphthyl and 2-naphthyl. When indicated, carbon atoms present within a carbocyclic ring may be optionally substituted with any of variety of ring substituents, as described above, or with specifically listed substituents.

The term “arylalkyl” refers to an aryl group is linked via an alkyl group. Certain arylalkyl groups are (C₆-C₁₈aryl)C₁-C₈alkyl groups (i.e., groups in which a 6- to 18-membered aryl group is linked via a C₁-C₈alkyl group). Such groups include, for example, groups in which phenyl or naphthyl is linked via a bond or C₁-C₈alkyl, preferably via C₁-C₄alkyl, such as benzyl, 1-phenyl-ethyl, 1-phenyl-propyl and 2-phenyl-ethyl.

The term “aryloxy” refers to an aryl group linked via a carbonyl (i.e., a group having the general structure —C(═O)—O-aryl). Phenoxy is a representative aryloxy group.

“Biphenyl” as used herein indicates, for example, a group of the formula:

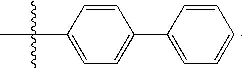

3-phenyl-phenyl and 2-phenyl-phenyl groups are also included in the definition of biphenyl. When indicated, the biphenyl group is substituted.

As used herein, the term “heteroaryl” is intended to indicate a stable 5-to 7-membered monocyclic or bicyclic or 7-to 10-membered bicyclic heterocyclic ring which contains at least 1 aromatic ring that contains from 1 to 4 heteroatoms selected from N, O, and S, with remaining ring atoms being carbon. When the total number of S and O atoms in the heteroaryl group exceeds 1, then these heteroatoms are not adjacent to one another. It is preferred that the total number of S and O atoms in the heterocycle is not more than 1, 2, or 3, more typically 1 or 2. It is particularly preferred that the total number of S and O atoms in the aromatic heterocycle is not more than 1. Examples of heteroaryl groups include pyridyl, furanyl, indolyl, pyrimidinyl, pyridizinyl, pyrazinyl, imidazolyl, oxazolyl, thienyl, thiazolyl, triazolyl, isoxazolyl, quinolinyl, pyrrolyl, pyrazolyl, and 5,6,7,8-tetrahydroisoquinoline.

The term “heterocyclic group” or “heterocycle” is used to indicate saturated, partially unsaturated, or aromatic groups having 1 or 2 rings, 3 to 8 atoms in each ring and in at least one ring between 1 and 3 heteroatoms selected from N, O, and S. Any nitrogen or sulfur heteroatoms may optionally be oxidized. The heterocyclic group may be attached to its pendant group at any heteroatom or carbon atom that results in a stable structure. The heterocyclic groups described herein may be substituted on a carbon or nitrogen atom if the resulting compound is stable. A nitrogen in the heterocycle may optionally be quaternized.

Representative examples of heteroaryl groups and heterocyclic groups include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl;-1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

An “inverse agonist” of the C5a receptor is a compound which inhibits the activity of C5a at the C5a receptor, and reduces the activity of the C5a receptor below its basal activity level in the absence of added C5a. Inverse agonists of the C5a receptor may also inhibit binding of C5a to the C5a receptor. The ability of a compound to inhibit the binding of C5a to the C5a receptor may be measured by a binding assay, such as the radioligand binding assay given in Example 5. The basal activity of the C5a receptor may be determined from a GTP binding assay, such as the assay of Example 6. The reduction of C5a activity may also be determined from a GTP binding assay such as the assay of Example 6 or a calcium mobilization assay such as the assay of Example 7.

A “neutral antagonist of the C5a receptor is a compound which inhibits the activity of C5a at the C5a receptor, but does not significantly change the basal activity of the C5a receptor. Neutral antagonists of the C5a receptor may inhibit the binding of C5a to the C5a receptor.

A “partial agonist” of the C5a receptor elevates the activity of the C5a receptor above the basal activity level of the receptor in the absence of C5a, but does not elevate the activity of the C5a receptor to the level brought about by saturating levels of the natural agonist, C5a. Partial agonist compounds may inhibit the binding of C5a to the C5a receptor. Partial agonists of the C5a receptor usually elevate the active of the C5a receptor from 5% to 90% of the activity level brought about by saturated concentrations of the natural agonist, C5a.

As used herein, a “pharmaceutically acceptable salt” is an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4 and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein, including those listed by Remington's Pharmaceutical Sciences, 17th ed., Mack Publishing Company, Easton, Pa., p. 1418 (1985). Accordingly, the present disclosure should be construed to include all pharmaceutically acceptable salts of the compounds specifically recited. A wide variety of synthetic procedures is available for the preparation of pharmaceutically acceptable salts. In general, a pharmaceutically acceptable salt can be synthesized from a parent compound that contains a basic or acidic moiety by any conventional chemical method. Briefly, such salts can be prepared by reacting the free acid or base forms of these compounds with a stoichiometric amount of the appropriate base or acid in water, an organic solvent, or a mixture of the two; generally, nonaqueous media like ether, ethyl acetate, ethanol, isopropanol or acetonitrile are preferred.

“A C5a receptor” is a G-coupled protein receptor that specifically binds C5a protein. Preferably the C5a receptor is a human C5a receptor such as the protein product of the sequence of the resulting PCR product described by Gerard and Gerard, (1991) Nature 349:614-17. The human C5a receptor may also be that described by Boulay (1991) Biochemistry, 30(12): 2993-9 (GENBANK Accession No. M62505). Non-primate C5a receptors may be a rat C5a receptor such as a rat C5a receptor, GENBANK Accession Nos. X65862, Y09613, and AB003042, a canine C5a receptor, GENBANK Accession No. X65860, or a guinea pig C5a receptor, GENBANK Accession No. U86103.

A “C5a receptor modulatory amount” of a compound is an amount that is sufficient to yield a plasma concentration of the compound (or its active metabolite, if a prodrug) high enough to detectably alter (modulate) C5a receptor activity and/or ligand binding, when that concentration is used in an in vitro assay. Suitable in vitro assays include the standard in vitro C5 receptor-mediated chemotaxis assay (described in Example 8 herein); C5a receptor-mediated calcium mobilization assay (described in Example 7 herein); and/or radioligand binding assay such as the assay provided in Example 5.

A “therapeutically effective amount” of a drug or pharmaceutical agent that elicits a detectable and desirable biological or medical response in a patient. Such a biological or medical response may take place in a tissue, a system, a non-human animal or a human and is generally sought by a researcher, veterinarian, medical doctor or other clinician. For example, a therapeutically effective amount may reduce symptom severity or frequency. Alternatively, or in addition, a therapeutically effective amount may improve patient outcome and/or prevent or delay disease or symptom onset. The dosage regimen utilizing an C5a antagonist in combination with a C5a receptor-inactive therapeutic agent is selected in accordance with a variety of factors including type, species, age, weight, sex and medical condition of the patient; the severity of the condition to be treated; the route of administration; the renal and hepatic function of the patient; and the particular compound or salt or ester thereof employed. Since two different active agents are being used together in a combination therapy, the potency of each of the agents and the enhanced effects achieved by combining them together (if any) must also be taken into account in determining a therapeutically effective amount. One marker of pathogenic inflammation is C reactive protein (CRP). Decreased plasma levels of CRP after treatment (as compared to levels before treatment) is an indication of an effective anti-inflammatory treatment of a disease with an inflammatory component.

A “patient” is any individual treated with a C5a antagonist as provided herein. Patients include humans, as well as other animals such as companion animals (e.g., dogs and cats) and livestock. Patients may be experiencing one or more symptoms of a condition responsive to C5a receptor modulation, or may be free of such symptom(s) (i.e., treatment may be prophylactic). In certain embodiments, the patient is a human.

As used herein a “patient at risk for myocardial infarction or stroke” is any patient determined to have one or more of the known risk factors for such vascular events. Known risk factors for myocardial infarction and stroke include, but are not limited to obesity, an elevated cholesterol level, hypertension, elevated low density lipoprotein (LDL) levels, congenital heart defects, smoking, previous history of myocardial infarction or stroke, and sedentary lifestyle.

A “prodrug” is a compound that may not fully satisfy the structural requirements of the compounds provided herein, but is modified in vivo, following administration to a patient, to produce a substituted tetrahydroisoquinoline. For example, a prodrug may be an acylated derivative of a compound as provided herein. Prodrugs include compounds wherein hydroxy, amine, or sulfhydryl groups are bonded to any group that, when administered to a mammalian subject, cleaves to form a free hydroxyl, amino, or sulfhydryl group, respectively. Examples of prodrugs include, but are not limited to, acetate, formate, and benzoate derivatives of alcohol and amine functional groups within the compounds provided herein. Preferred prodrugs include acylated derivatives. Prodrugs may be prepared by modifying functional groups present in the compounds in such a way that the modifications are cleaved to the parent compounds. Those of ordinary skill in the art will recognize various synthetic methods that may be employed to prepare prodrugs of the compounds provided herein.

C5A Antagonists

Any C5a antagonist, including neutral antagonists and inverse agonists, may be used in the compositions and methods provided herein. The synthesis of certain C5a antagonists listed herein has been described in PCT International Application WO 02/49993 and U.S. patent application Ser. No. 09/672,701 at pages 161-201 which is hereby incorporated by reference for its teachings regarding the synthesis of C5a antagonists. Additional C5a antagonists have been described in PCT International Application WO 02/14265, published Feb. 21, 2002. As noted above, compounds that act as C5a antagonists may be readily identified by assays for C5a receptor antagonist activity such as the assays provided in Examples 7 and 8, herein. The C5a antagonist may also be chosen from the compounds given in Table I and their pharmaceutically acceptable salts.

Certain C5a antagonists have a molecular mass of less than 700 a.m.u. and exhibit a three dimensional structure that is described by Formula I.

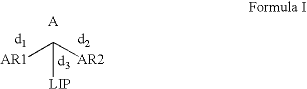

wherein:

AR1 and AR2 are independently carbocyclic aryl or heteroaryl;

LIP represents an alkyl, carbocyclic aryl, heteroaryl, or arylalkyl;

A is oxygen or nitrogen;

d₁ represents the distance between A and the geometric center of AR1 and is between 3 and 6 angstroms in at least one energetically accessible conformer of the compound;

d₂ represents the distance between A and the geometric center of AR2 and is between 7 and 10 angstroms in at least one energetically accessible conformer of the compound; and

d₃ represents the distance between A and the nearest atom of LIP and is between 3 and 6 angstroms in at least one energetically accessible conformer of the compound.

Preferred C5a antagonists of Formula I are non-peptidic and contain one or more heteroaryl rings.

Within certain combinations provided herein, the C5a antagonist is a compound of Formula II:

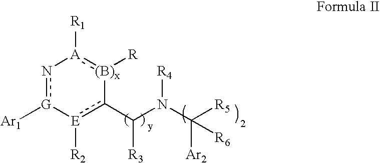

and the pharmaceutically acceptable salts thereof.

The ring system in Formula II represented by

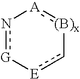

is (i) a 5-membered heteroaryl ring system, in which x is 0; A is m carbon, nitrogen, oxygen, or sulfur; and E and G are independently carbon or nitrogen, provided that the 5-membered heteroaryl ring system does not contain more than 3 heteroatoms or more than 1 oxygen or sulfur atom; or (ii) a 6-membered heteroaryl ring system, in which x is 1; A, B, E, and G are independently chosen from carbon and nitrogen, provided that the 6-membered heteroaryl ring system does not contain more than 3 nitrogen atoms.

R and R₁ in Formula II independently represent: i) hydrogen, hydroxy, halogen, amino, cyano, nitro, —CHO, —CONH₂, C₁-C₆haloalkyl, or C₁-C₆haloalkoxy; ii) C₁-C₆alkyl, C₁-C₆alkenyl, C₁-C₆alkynyl, C₁-C₆alkoxy, C₃-C₇cycloalkyl, (C₃-C₇cycloalkyl)C₁-C₄alkyl, mono- or di-(C₁-C₆alkyl)amino, mono- or di-(C₁-C₆alkyl)aminoC₁-C₆alkyl, mono- or di-(C₁-C₆alkyl)carboxamide, C₁-C₆alkoxycarbonyl, —NHSOₙC₁-C₆alkyl, —SOₙN(C₁-C₆alkyl)(C₁-C₆alkyl), or phenyl-SOₙ—, each of which is optionally substituted, and wherein n is 0, 1 or 2; or iii) naphthyl, phenyl, phenylC₁-C₄carbhydryl, a 5- or 6-membered heteroaryl group, or a 5- or 6-membered heteroarylC₁-C₄carbhydryl group, each of which is optionally substituted.

If E is nitrogen, R₂ is chosen from C₁-C₇alkyl, C₂-C₇alkenyl, C₂-C₇alkynyl, C₃-C₇cycloalkyl(C₁-C₄alkyl), benzyl and C₁-C₆haloalkyl. If E is Carbon, R₂ is chosen from halogen, hydroxy, C₁-C₇alkyl, C₂-C₇alkenyl, C₂-C₇alkynyl, C₁-C₇alkoxy, C₁-C₇alkylamino, C₃-C₇cycloalkyl(C₁-C₄alkyl), benzyl, C₁-C₆haloalkyl, and C₁-C₆haloalkoxy.

R₃ is hydrogen, C₁-C₆alkyl, C₂-C₆alkenyl, hydroxyC₁-C₆alkyl, C₁-C₆haloalkyl, C₃-C₇cycloalkyl, (C₃-C₇cycloalkyl)C₁-C₄alkyl, or phenyl(C₁-C₄alkyl), or when x is 0, R₁ and R₃ may be joined to form a C₃-C₇cycloalkyl ring, which may be optionally substituted. y is and integer ranging from 1 to 6; in certain embodiments y is 1 or 2.

R₄ represents i) hydrogen; ii) C₁-C₆alkyl, C₂-C₆alkenyl, C₂-C₆alkynyl, C₃-C₇cycloalkyl, C₃-C₇cycloalkenyl, (C₃-C₇cycloalkyl)C₁-C₄alkyl, (C₃-C₇cycloalkenyl)C₁-C₄alkyl, or hexahydro-1,3-benzodioxolylmethyl, each of which may be optionally substituted; iii) optionally substituted arylC₁-C₄alkyl having 1 or 2 fused or pendant rings; iv) optionally substituted arylC₁-C₄alkyl, wherein the aryl portion is fused to a 5- to 7-membered saturated or partially unsaturated ring having 0, 1, or 2 ring atoms chosen from N, O, and S with remaining ring atoms being carbon; v) optionally substituted heterocycloalkyl(C₀-C₄alkyl); vi) optionally substituted heteroarylC₁-C₂alkyl having 1 or 2 fused or pendant rings, from 5 to 7 members in each ring, and in at least one ring 1 to 3 heteroatoms selected from N, O, and S; or vii) optionally substituted saturated or partially unsaturated heterocycle(C₀C₄alkyl) having from 4 to 7 ring members, 1 or 2 of which ring members are N, S or O, with remaining ring members being carbon.

R₅ and R₆ are independently chosen from hydrogen and C₁-C₆alkyl, and z is 1, 2, or 3.

Ar₁ is optionally substituted aryl having 1 or 2 fused or pendant rings, optionally substituted phenyl fused to a 5- to 7-membered saturated or partially unsaturated ring having 0, 1, or 2 ring atoms chosen from N, O, and S with remaining ring atoms being carbon, or optionally substituted heteroaryl, having 1 or 2 fused or pendant rings, from 5 to 7 members in each ring, and in at least one ring 1 to 3 heteroatoms selected from N, O, and S.

Ar₂ is optionally substituted C₃-C₇cycloalkyl, C₃-C₇cycloalkyl(C₁-C₄alkyl), C₃-C₇cycloalkenyl, C₃-C₇cycloalkenyl(C₁-C₄alkyl), or hexahydro-1,3-benzodioxolyl, optionally substituted aryl having 1 or 2 fused or pendant rings, optionally substituted phenyl fused to a 5- to 7-membered saturated or partially unsaturated ring having 0, 1, or 2 ring atoms chosen from N, O, and S with remaining ring atoms being carbon, or optionally substituted heteroaryl having 1 or 2 fused or pendant rings, from 5 to 7 members in each ring, and in at least one ring from 1 to 3 heteroatoms selected from the group consisting of N, O, and S.

Certain compounds and salts of Formula II are imidazoles in which A and G are carbon, E is nitrogen, X is 0 and R₁ and R₃ do not form a ring. Such compounds satisfy Formula IIa:

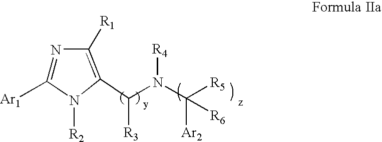

Other compounds and salts of Formula II are 5-aryl-pyrazoles or 1-aryl pyrazoles, in which E is carbon, one of A and G is nitrogen (with the other being carbon), x is 0, and R₁ and R₃ are not joined to form a cycloalkyl ring. Such compounds satisfy Formula IIB or IIc:

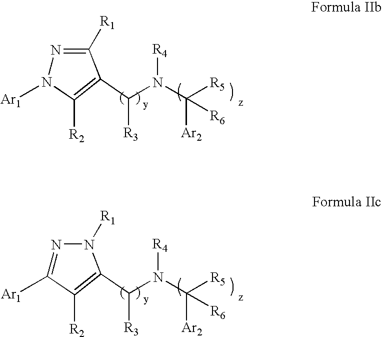

Additional compounds and salts of Formula II are triazoles in which in which A and E are nitrogen, G is carbon, x is 0, and R₁ and R₃ are not joined to form a cycloalkyl ring. Such compounds satisfy Formula IId:

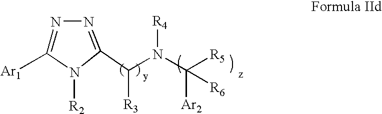

Further compounds and salts of Formula II are isothiazoles or isoxazoles in which x is 0, A is sulfur or oxygen, G and E are carbon; and R₁ and R₃ are not joined to form a cycloalkyl ring. Such compounds satisfy Formula IIe or IIf:

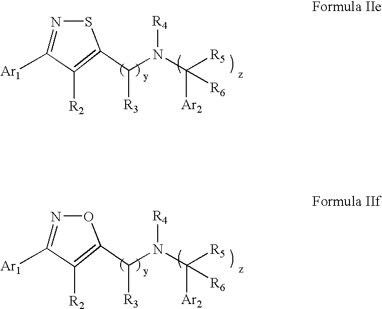

Additional compounds and salts of Formula II are pyridines in which x is 1, A, B, E, and G are carbon, and R₁ and R₃ are not joined to form a cycloalkyl ring. Such compounds satisfy Formula IIg:

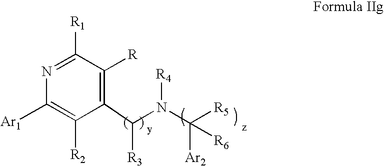

Still further compounds and salts of Formula II are pyrimidines or pyridizines in which x is 1, either A or B is nitrogen, and E and G are carbon. Such compounds satisfy Formula IIh or Iii.

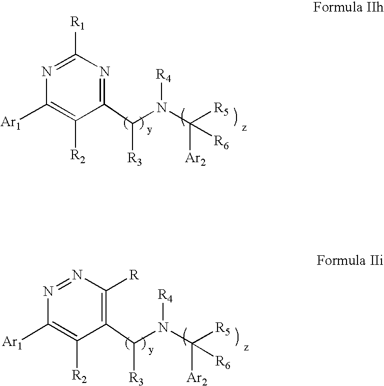

Compounds and salts of Formula II also include those in which R₁ and R₃ are joined to form a cycloalkyl ring. Certain such compounds are illustrated by Formula IIj, in which x is 0; y is 1, A and G are carbon, and E is nitrogen. X, in Formula IIj, represents from 1 to 4 optional substituents independently chosen from hydroxy, halogen, cyano, C₁-C₂alkyl, C₁-C₂alkoxy, C₁-C₂haloalkyl, and C₁-C₂haloalkoxy, and a is 1, 2 or 3

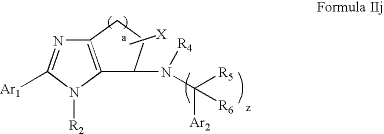

Within certain embodiments, compounds and salts of Formula II and subformulas thereof satisfy one or more of the following:

z is 1; R₅ is hydrogen; and R₆ is hydrogen, methyl, or ethyl.

z is 1; R₅ is hydrogen; R₆ is hydrogen, methyl, or ethyl; and Ar₁ is phenyl, pyrazolyl, or thienyl, each of which is optionally substituted with 1 or 2 groups independently chosen from halogen, hydroxy, C₁-C₂alkyl, C₁-C₂alkoxy, C₁-C₂haloalkyl, and C₁-C₂haloalkoxy.

z is 1; R₅ and R₆ are hydrogen; and Ar₁ is unsubstituted phenyl, unsubstituted pyrazolyl, or unsubstituted thienyl.

Representative R₁ groups include phenyl substituted with from 0 to 4 groups independently chosen from hydroxy, halogen, amino, cyano, nitro, —COOH, —CONH₂, —SO₂NH₂, C₁-C₆ haloalkyl, C₁-C₆ haloalkoxy, C₁-C₆ alkyl, C₁-C₆alkoxy, 1,3-dioxol-5-yl, C₁-C₆alkanoyl, C₁-C₆alkylsulfonyl, C₁-C₆alkylsulfinyl, C₁-C₆alkylthio, C₂-C₆alkanone; C₁-C₆alkanoyl; C₂-C₆alkyl ether; C₁-C₆ alkanoyloxy; C₁-C₆alkoxycarbonyl, and C₁-C₆alkylcarboxamide. For example, R₁ may be phenyl substituted with from 0 to 2 groups independently chosen from hydroxy, halogen, amino, cyano, nitro, —COOH, —CONH₂, —SO₂NH₂, C₁-C₂ haloalkyl, C₁-C₂ haloalkoxy, C₁-C₂alkyl, and C₁-C₂alkoxy. In certain embodiments, R₁ is unsubstituted phenyl. Additional representative R₁ groups include thienyl and pyridyl, each of which is substituted with from 0 to 2 groups independently chosen from hydroxy, halogen, amino, cyano, nitro, —COOH, —CONH₂, —SO₂NH₂, C₁-C₂ haloalkyl, C₁-C₂ haloalkoxy, C₁-C₂alkyl, and C₁-C₂alkoxy. In other embodiments, R₁ is hydrogen, halogen, C₁-C₄alkyl, C₁-C₄alkoxy, cyano, trifluoromethyl, pentafluoroethyl, difluoromethyl, trifluoromethoxy, difluoromethoxy, 1,1-difluoroethyl, C₁-C₂alkylaminoC₁-C₂alkyl, hydroxymethyl, and hydroxyethyl.

Representative R₂ groups include hydrogen, propyl, butyl, pentyl and 3-methylbutyl; R₂ is preferably hydrogen when Ar₁ is alkyl-substituted phenyl, pyrazolyl or phenyl, and E is carbon.

Representative R₃ groups include hydrogen and C₁-C₅alkyl.

R₄, within certain embodiments, is C₁-C₆alkyl, C₂-C₆alkenyl, C₂-C₆alkynyl, C₃-C₇cycloalkyl, C₃-C₇cycloalkenyl, (C₃-C₇cycloalkyl)C₁-C₄alkyl, (C₃-C₇cycloalkenyl)C₁-C₄alkyl, or hexahydro-1,3-benzodioxolylmethyl, each of which is substituted with from 0 to 3 substituents independently chosen from hydrogen, hydroxy, halogen, amino, cyano, C₁-C₂alkyl, C₁-C₂alkoxy, and C₁-C₂alkoxycarbonyl. Certain such R₄ groups are unsubstituted, such as cyclopentyl, cyclohexyl, cyclohexenyl, cyclohexylmethyl, cyclohexenylmethyl, cyclhexenyl, or hexahydro-1,3-benzodioxolylmethyl. In other embodiments, R₄ is: a) aryl or aryl(C₀-C₂)alkyl having 1 or 2 fused or pendant rings, b) benzyl fused to a 5- to 7-membered saturated or partially unsaturated ring having 0, 1, or 2 ring atoms chosen from N, O, and S with remaining ring atoms being carbon, c) saturated or partially unsaturated heterocycle(C₀-C₄alkyl) having 1 or 2 fused or pendant rings, from 5- to 7-members in each ring, and in at least one ring 1 to 3 heteroatoms selected from N, O, and S or d) heteroaryl or heteroaryl(C₁-C₂alkyl) having 1 or 2 fused or pendant rings, from 5- to 7-members in each ring, and in at least one ring 1 to 3 heteroatoms selected from N, O, and S, wherein each of a), b), c), and d) are substituted with from 0 to 4 groups independently chosen from hydroxy, halogen, amino, cyano, nitro, —COOH, —CONH₂, —SO₂NH₂, oxo, C₁-C₆ haloalkyl, C₁-C₆ haloalkoxy, C₁-C₆ alkyl, C₁-C₆alkoxy, C₁-C₆alkanoyl, C₁-C₆sulfonate, C₁-C₆alkylsulfonyl, C₁-C₆alkylsulfinyl, C₁-C₆alkylthio, C₁-C₆alkanone, C₂-C₆alkyl ether, C₁-C₆ alkanoyloxy, C₁-C₆alkoxycarbonyl, and C₁-C₆alkylcarboxamide. For example, R₄ may be optionally substituted benzyl. Alternatively, R₄ may be pyridylmethyl, pyrimidylmethyl, thienylmethyl, napthylmethyl, indolylmethyl, benzoxadiazolylmethyl (e.g., benzoxadiazol-5-ylmethyl), benzooxazolylmethyl, benzothiazolyl, quinazolinylmethyl, or benzimidazolylmethyl, each of which is substituted with from 0 to 2 groups independently chosen from hydroxy, halogen, amino, cyano, C₁-C₂haloalkyl, C₁-C₂haloalkoxy, C₁-C₂ alkyl, mono- or di-(C₁-C₂)alkylamino, and C₁-C₂alkoxy. In further embodiments, R₄ is 1,3-benzodioxol-5-ylmethyl, 2,3-dihydro-1-benzofuran-6-ylmethyl, 2,3-dihydro-1-benzofuran-5-ylmethyl, 2,3-dihydro-1,4-benzodioxin-6-ylmethyl, chroman-6-ylmethyl, chroman-7-ylmethyl, 1,3-benzothiazolylmethyl, 2,3-dihydroindol-5-ylmethyl, each of which is substituted with from 0 to 2 substituents independently selected from hydroxy, halogen, amino, cyano, oxo, C₁-C₂haloalkyl, C₁-C₂haloalkoxy, C₁-C₂ alkyl, mono- or di-(C₁-C₂)alkylamino, and C₁-C₂alkoxy. In certain embodiments in which R₄ is heteroaryl or heteroaryl(C₁-C₂alkyl), the heteroaryl group is pyridyl, pyrimidyl, thienyl, naphthyl, indolyl, benzoxadiazolyl, benzoxazolyl, quinazolinyl, or benzimidazolyl, each optionally substituted with from 1 to 2 groups independently chosen from hydroxy, halogen, amino, cyano, C₁-C₂haloalkyl, C₁-C₂haloalkoxy, C₁-C₂ alkyl, and C₁-C₂alkoxy.

Representative Ar₂ groups include C₃-C₇cycloalkyl (e.g., cyclopentyl or cyclohexyl), C₃-C₇cycloalkenyl (e.g., cyclohexenyl), (C₃-C₇cycloalkyl) C₁-C₄alkyl, (C₃-C₇cycloalkenyl)C₁-C₄alkyl, and hexahydro-1,3-benzodioxolyl, each of which is optionally substituted with from 0 to 3 groups independently chosen from hydrogen, hydroxy, halogen, amino, cyano, C₁-C₂alkyl, C₁-C₂alkoxy, or C₁-C₂alkoxycarbonyl. Further Ar₂ groups include phenyl substituted with from 0 to 4 groups independently chosen from hydroxy, halogen, amino, cyano, nitro, —COOH, —CONH₂, —SO₂NH₂, oxo, C₁-C₆haloalkyl, C₁-C₆haloalkoxy, C₁-C₆alkyl, C₁-C₆alkoxy, mono- and di-(C₁-C₆alkyl)amino, C₁-C₆alkanoyl, C₁-C₆alkylsulfonyl, C₁-C₆alkylsulfinyl, C₁-C₆alkylthio, C₂-C₆alkanone, C₂-C₆alkylether, C₁-C₆alkanoyloxy, C₁-C₆alkoxycarbonyl, C₁-C₆alkylcarboxamide, C₂-C₆cycloalkylamino, and C₂-C₆cycloalkylamino(C₁-C₄alkyl). Still further Ar₂ groups include pyridyl, pyrimidyl, thienyl, naphthyl, indolyl, benzoxadiazolyl (e.g., benzoxadiazol-5-yl), benzoxazolyl, quinazolinyl, and benzimidazolyl, each of which is substituted with from 0 to 2 groups independently chosen from hydroxy, halogen, amino, cyano, C₁-C₂haloalkyl, C₁-C₂haloalkoxy, C₁-C₂alkyl, mono- or di-(C₁-C₂alkyl)amino, C₁-C₂alkoxy, and C₂-C₆cycloalkylamino. Additional representative Ar₂ groups include phenyl fused to a 5- to 7-membered saturated or partially unsaturated ring having 0, 1, or 2 ring atoms chosen from N, O, and S with remaining ring atoms being carbon, or a heteroaryl or heteroaryl(C₁-C₂alkyl) group, having from 1 to 2 fused or pendant rings, from 5 to 7 members in each ring, and in at least one ring 1 to 3 heteroatoms selected from N, O, and S, each of which is substituted with from 0 to 4 groups independently chosen from hydroxy, halogen, amino, cyano, nitro, —COOH, —CONH₂, —SO₂NH₂, oxo, C₁-C₆haloalkyl, C₁-C₆haloalkoxy, C₁-C₆alkyl, C₁-C₆alkoxy, C₁-C₆alkanoyl, C₁-C₆sulfonate, C₁-C₆alkylsulfonyl, C₁-C₆alkylsulfinyl, C₁-C₆alkylthio, C₂-C₆alkanone, C₂-C₆alkyl ether, C₁-C₆ alkanoyloxy, C₁-C₆alkoxycarbonyl, and C₁-C₆alkylcarboxamide. For example, the phenyl group fused to a 5-to 7-membered saturated or partially unsaturated ring may be 1,3-benzodioxol-5-yl, 2,3-dihydro-1-benzofuran-6-yl, 2,3-dihydro-1-benzofuran-5-yl, 2,3-dihydro-1,4-benzodioxin-6-yl, chroman-6-yl, chroman-7-yl, 1,3-benzothiazolyl, or 2,3-dihydroindol-5-yl, each of which is substituted with from 0 to 2 substituents independently selected from hydroxy, halogen, amino, cyano, oxo, C₁-C₂haloalkyl, C₁-C₂haloalkoxy, C₁-C₂ alkyl, and C₁-C₂alkoxy.

Formulas IIk-IIp represent additional subformulas of compounds within the scope of Formula II.

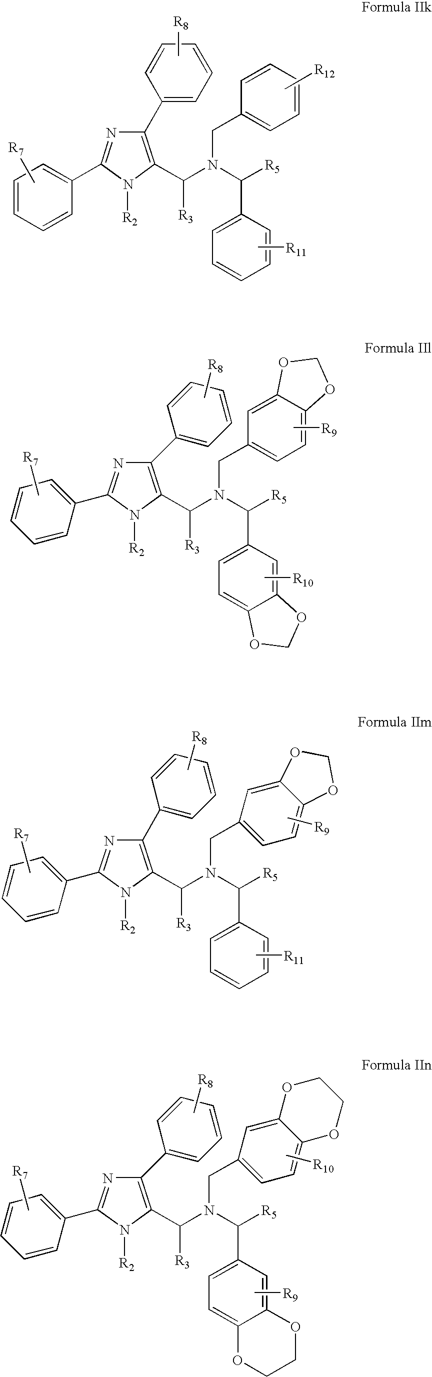

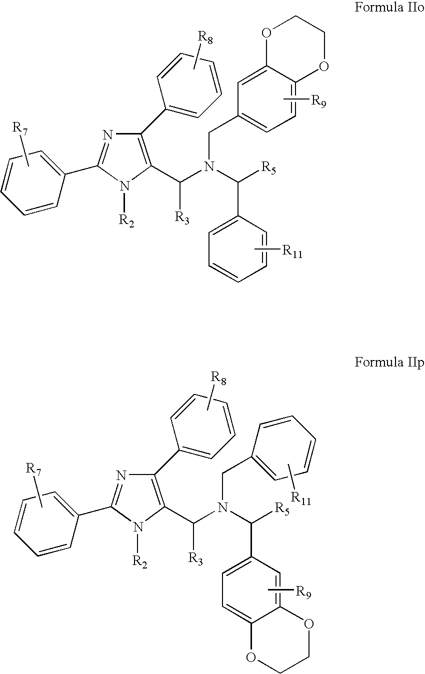

Within Formulas Ilk-IIp, R₂ is C₃-C₅ alkyl; R₃ is hydrogen or methyl; R₅ is hydrogen or methyl; R₇ represents from 0 to 3 substituents independently chosen from hydroxy, cyano, halogen, methyl, ethyl, methoxy, and ethoxy; R₈ represents from 0 to 3 substituents independently chosen from halogen, hydroxy, nitro, methyl, ethyl, methoxy, ethoxy, trifluoromethyl, difluoromethyl, trifluoromethoxy, difluoromethoxy, —CONH₂, —OC(═O)CH₃, —COOH, methylthio, ethylthio, and —SO₂CH₃; R₉ and R₁₀ may occur at any position on the benzo[1,3]dioxole or 2,3-Dihydro-benzo[1,4]dioxine group available for substitution and represent from 0 to 3 substituents independently chosen from halogen, methyl, and methoxy; R₁₁ and R₁₂ independently represent from 0 to 3 substituents independently chosen from halogen, hydroxy, nitro, methyl, ethyl, methoxy, ethoxy, trifluoromethyl, difluoromethyl, pentafluoroethyl, —CF₂CHF₂, trifluoromethoxy, difluoromethoxy, pentafluoroethoxy, —OCF₂CHF₂, —CONH₂, —C(═O)OCH₃, —OC(═O)CH₃, —COOH, methylthio, ethylthio, —SO₂NH₂, and —SO₂CH₃.

[Redo and Expand the Formula III Section After Completing 3003 Claims]

The present invention further comprises combinations in which the C5a receptor antagonist is a compound of Formula III:

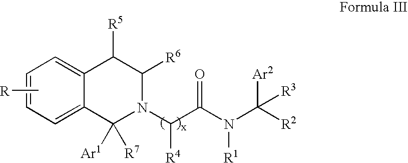

or a pharmaceutically acceptable salt thereof, wherein:

x is 1, 2, or 3.

R represents up to 4 groups independently chosen from hydrogen, halogen, hydroxy, optionally substituted alkoxy, optionally substituted alkyl, optionally substituted alkenyl, optionally substituted alkynyl, cyano, amino, nitro, —COOH, carboxamide, optionally substituted mono or di-alkyl amino, optionally substituted haloalkyl, and optionally substituted haloalkoxy.

R¹ for compounds of Formula I is selected from the group consisting of optionally substituted alkyl, optionally substituted alkenyl, optionally substituted alkynyl, optionally substituted cycloalkyl, optionally substituted (cycloalkyl)alkyl, optionally substituted aryl, optionally substituted heteroaryl, optionally substituted (aryl)alkyl, optionally substituted (heteroaryl)alkyl, and optionally substituted indanyl.

R², R³, and R⁴ are independently selected at each occurrence from the group consisting of hydrogen, optionally substituted alkyl, halogen, and optionally substituted alkoxy.

R⁵ and R⁶ are independently selected from the group consisting of hydrogen, halogen, optionally substituted alkyl, optionally substituted alkenyl, optionally substituted alkynyl, optionally substituted alkoxy, optionally substituted haloalkyl, optionally substituted haloalkoxy, hydroxy, amino, mono or dialkyl amino, and cyano.

R⁷ is hydrogen, optionally substituted alkyl, optionally substituted alkenyl, optionally substituted alkynyl, optionally substituted alkoxy, or optionally substituted arylalkyl.

Ar¹ is an optionally substituted phenyl, optionally substituted naphthyl, optionally substituted biphenyl, optionally substituted heterocyclic group, or and optionally substituted phenyl group fused to a 5 to 7 membered saturated or partially unsaturated ring, said saturated or partially unsaturated ring having from 5 to 7 ring atoms, with 0, 1, or 2 ring atoms chosen from N, O, and S with remaining ring atoms being carbon.

Alternatively, Ar¹ is taken in combination with CR⁷ (CR⁷Ar¹), to form an optionally substituted group of the formula:

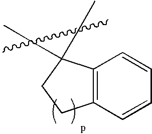

wherein p is an integer from 1 to about 3.

Ar² is optionally substituted aryl or optionally substituted heteroaryl having about 5 to 7 ring atoms and between 1 and 3 ring heteroatoms selected from N, O, and S.

Other C5a antagonists for use in the combination provided by the invention include compounds of Formula IV:

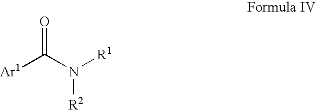

or a pharmaceutically acceptable salt thereof.

Ar¹ is, in Formula IV: i) optionally substituted phenyl having at least one optionally substituted phenyl or optionally substituted heterocyclic substituent attached thereto; ii) optionally substituted carbocycle having from 2 to about 4 partially unsaturated or aromatic rings, 3 to 8 members in each ring, or iii) optionally substituted heteroaryl.

R¹ in formula IV is optionally substituted cycloalkyl, optionally substituted (cycloalkyl)alkyl, optionally substituted (heteroaryl)alkyl, optionally substituted (aryl)alkyl, optionally substituted aryl, optionally substituted heteroaryl having about 5 to 7 ring atoms and between 1 and 3 ring heteroatoms selected from N, O, and S, or optionally substituted (aryl)alkyl, wherein the aryl portion is fused to a 5- to 7-membered saturated or partially unsaturated ring that (a) has 0, 1 or 2 ring atoms independently chosen from N, O and S, with remaining ring atoms being carbon, and (b) is substituted with from 0 to 2 substituents independently chosen from halogen, alkyl and alkoxy.

R² in Formula IV is alkyl, cycloalkyl, (cycloalkyl)alkyl, heteroaryl, (heteroaryl)alkyl, aryl, (aryl)alkyl, or indanyl, each of which is optionally substituted, or R² is optionally substituted phenyl(Co-C₂alkyl), wherein the phenyl portion is fused to a 5 to 7 membered saturated or partially unsaturated ring that (a) has 0, 1 or 2 ring atoms independently chosen from N, O and S, with remaining ring atoms being carbon, and (b) is substituted with from 0 to 3 substituents independently chosen from halogen, alkyl, alkoxy, haloalkyl, and haloalkoxy. For example, in certain embodiments R² is indanyl, (aryl)alkyl, or cycloalkyl, each of which is optionally substituted.

In certain embodiments of compounds of Formula IV:

Ar¹ is selected from:

i) phenyl having at least one phenyl substituent or heterocyclic substituent attached thereto, wherein each phenyl, phenyl substituent, or heterocyclic substituent is substituted with from 0 to 4 substituents independently selected from halogen, amino, cyano, hydroxy, C₁-C₄alkyl, C₁-C₄alkoxy, C₁-C₂ haloalkyl, C₁-C₂ haloalkoxy, mono- and di-C₁-C₄alkylamino, C₁-C₂ alkylthio, and —NHC(═O)C₁-C₂ alkyl;

ii) naphthyl substituted with from 1 to 3 substituents independently selected from halogen, amino, cyano, hydroxy, C₁-C₄alkyl, C₁-C₄alkoxy, C₁-C₂ haloalkyl, C₁-C₂ haloalkoxy, mono- and di-C₁-C₄alkylamino, C₁-C₂ alkylthio, —NHC(═O)C₁-C₂ alkyl, optionally substituted phenyl, and optionally substituted thienyl; and

iii) 9h-fluorenyl susbstituted with from 0 to 3 substituents independently selected from halogen, amino, cyano, hydroxy, C₁-C₄alkyl, C₁-C₄alkoxy, C₁-C₂ haloalkyl, C₁-C₂ haloalkoxy, mono- and di-C₁-C₄alkylamino, C₁-C₂ alkylthio, —NHC(═O)C₁-C₂ alkyl; and

iv) heteroaryl substituted susbstituted with from 0 to 3 substituents independently selected from halogen, amino, cyano, hydroxy, C₁-C₄alkyl, C₁-C₄alkoxy, C₁-C₂ haloalkyl, C₁-C₂ haloalkoxy, mono- and di-C₁-C₄alkylamino, C₁-C₂ alkylthio, —NHC(═O)C₁-C₂ alkyl;

R¹ is:

i) (heteroaryl)C₀-C₄alkyl or (aryl) C₀-C₄alkyl, each of which is substituted with from 0 to 3 substituents independently selected from halogen, hydroxy, nitro, cyano, C₁-C₄ alkyl, C₂-C₄alkenyl, C₂-C₄alkynyl, C₁-C₄alkoxy, C₁-C₄alkylthio, —NC(═O)C₁-C₂alkyl, mono- and di(C₁-C₂alkyl)amino, C₂-C₃alkanoyloxy, C₁-C₃alkoxycarbonyl, C₁-C₂ haloalkyl, C₁-C₂haloalkoxy, thienyl, and phenyl; or

ii) (aryl)C₁-C₄alkyl substituted with from 0 to 5 substituents independently chosen from halogen, hydroxy, C₁-C₂ alkyl, C₁-C₂ alkoxy, C₁-C₂haloalkyl, and C₁-C₂haloalkoxy, wherein the aryl portion is fused to a 5- to 7-membered saturated or partially unsaturated ring that (a) has 0, 1 or 2 ring atoms independently chosen from N, O and S, with remaining ring atoms being carbon, and (b) is substituted with from 0 to 2 substituents independently chosen from halogen, C₁-C₄alkyl and C₁-C₄alkoxy; and

R² is selected from C₃-C₇ cycloalkyl, (C₃-C₇cycloalkyl)C₁-C₄alkyl, (heteroaryl)C₁-C₄alkyl, (aryl)C₁-C₄alkyl, and indanyl; each of which is substituted with from 0 to 5 substituents independently selected from halogen, hydroxy, C₁-C₄ alkyl, C₁-C₄ alkoxy, C₁-C₄alkylthio, —NC(═O)C₁-C₂alkyl, C₁-C₂haloalkyl, C₁-C₂haloalkoxy, thienyl, and phenyl.

Certain compounds and salts of Formula IV satisfy Formula IVa:

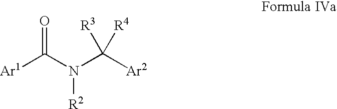

wherein:

Ar¹ is: i) phenyl substituted with from 0 to 4 substituents independently selected from halogen, hydroxy, C₁-C₄alkyl, C₁-C₄alkoxy, C₁-C₂ haloalkyl, C₁-C₂ haloalkoxy, C₁-C₂alkylthio, and —NHC(═O)C₁-C₂ alkyl, and substituted at the 2-position relative to the point of attachment with phenyl, thienyl, imidazolyl, pyrrolyl, pyrazolyl, oxazolyl, naphthyl, thiazolyl, or pyrimidinyl, each of which is substituted with from 0 to 3 substituents independently selected from halogen, amino, cyano, hydroxy, C₁-C₄alkyl, C₁-C₄alkoxy, C₁-C₂ haloalkyl, C₁-C₂haloalkoxy, mono- and di-C₁-C₄alkylamino, C₁-C₂ alkylthio, and —NHC(═O) C₁-C₂ alkyl, or

ii) naphthyl substituted with from 1 to 3 substituents independently selected from (a) halogen, amino, cyano, hydroxy, C₁-C₄alkyl, C₁-C₄alkoxy, C₁-C₂haloalkyl, C₁-C₂ haloalkoxy, mono- and di-C₁-C₄alkylamino, C₁-C₂alkylthio, and —NHC(═O)C₁-C₂ alkyl, and (b) phenyl and thienyl, each of which is substituted with from 0 to 3 substitutents indpendenlty chosen from halogen, C₁-C₂alkyl, C₁-C₂alkoxy, C₁-C₂haloalkyl, and C₁-C₂haloalkoxy; or

iii) 9H-fluorenyl substituted with from 0 to 3 substituents independently selected from halogen, amino, cyano, hydroxy, C₁-C₄alkyl, C₁-C₄alkoxy, C₁-C₂haloalkyl, C₁-C₂ haloalkoxy, mono- and di-C₁-C₄alkylamino, C₁-C₂alkylthio, and —NHC(═O)C₁-C₂ alkyl; or

iv) heteroaryl substituted with from 0 to 3 substituents independently selected from halogen, amino, cyano, hydroxy, C₁-C₄alkyl, C₁-C₄alkoxy, C₁-C₂haloalkyl, C₁-C₂haloalkoxy, mono- and di-C₁-C₄alkylamino, C₁-C₂alkylthio, and —NHC(═O)C₁-C₂ alkyl;

Ar² is: i) phenyl, ii) naphthyl, iii) a heterocycle having 1 or 2 rings, 3 to 8 atoms in each ring, and 1 to 3 heteroatoms independently selected from N, O, and S; or iv) phenyl fused to a 5- to 7-membered saturated or partially unsaturated ring having 0, 1, or 2 ring atoms independently chosen from N, O, and S, with remaining ring atoms being carbon; wherein each of i), ii), iii), and iv) is substituted with from 0 to 5 substituents independently selected from halogen, hydroxy, C₁-C₄alkyl, C₁-C₄alkoxy, C₁-C₂ haloalkyl, C₁-C₂ haloalkoxy, C₁-C₂ alkylthio, and —NHC(═O)C₁-C₂ alkyl;

R² is selected from C₃-C₇ cycloalkyl, (C₃-C₇cycloalkyl)C₁-C₄alkyl, (heteroaryl)C₁-C₄alkyl, (aryl)C₁-C₄alkyl, and indanyl; each of which is substituted with from 0 to 3 substituents independently selected from halogen, hydroxy, C₁-C₄ alkyl, C₁-C₄alkoxy, C₁-C₄alkylthio, —NC(═O)C₁-C₂alkyl, C₁-C₂ haloalkyl, C₁-C₂haloalkoxy, thienyl, and phenyl; and

R³ and R⁴ are independently selected from hydrogen, hydroxy, amino, cyano, halogen, C₁-C₄ alkyl, C₂-C₄alkenyl, and C₂-C₄alkynyl.

Further compounds and salts of Formula IV satisfy Formula IVb:

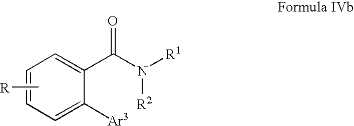

wherein:

Ar³ is phenyl, pyridyl, furanyl, thienyl, imidazolyl, pyrrolyl, pyrazolyl, oxazolyl, naphthyl, thiazolyl, or pyrimidinyl, each of which is substituted with from 0 to 3 substituents independently selected from halogen, hydroxy, amino, cyano, C₁-C₄alkyl, C₁-C₄alkoxy, mono- and di-(C₁-C₄alkyl)amino, C₁-C₂ haloalkyl, C₁-C₂haloalkoxy, C₁-C₂ alkylthio, and —NHC(═O)C₁-C₂ alkyl;

R⁶ represents from 0 to 4 substituents independently selected from halogen, hydroxy, C₁-C₄alkyl, C₁-C₄alkoxy, C₁-C₂haloalkyl, C₁-C₂haloalkoxy, C₁-C₂alkylthio, and —NHC(═O)C₁-C₂ alkyl; and

R¹ and R² are as described for Formula IV.

For example, certain such compounds satisfy Formula IVc:

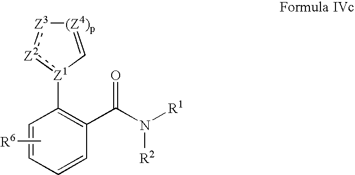

wherein Z¹ is carbon or nitrogen; Z², Z³, and each occurrence of Z⁴ are independently selected from CR⁷, NR⁸, S, and O such that each S or O ring atom, if any, is disposed between two CR⁷ groups; p is an integer ranging from 1 to about 3; R⁶ represents from 0 to 4 substituents independently selected from halogen, hydroxy, C₁-C₄alkyl, C₁-C₄alkoxy, C₁-C₂haloalkyl, C₁-C₂haloalkoxy, C₁-C₂ alkylthio, and —NHC(═O)C₁-C₂ alkyl; R⁷ is independently selected at each occurrence from hydrogen, halogen, hydroxy, amino, C₁-C₆alkyl, C₁-C₆alkoxy, C₁-C₆haloalkyl, C₁-C₆haloalkoxy, C₂-C₆alkenyl, C₂-C₆alkynyl, C₃-C₈cycloalkyl, mono-and di-(C₁-C₆alkyl)amino, cyano, nitro, and C₁-C₆alkanoyl; and R⁸ is independently selected at each occurrence from hydrogen, amino, C₁-C₆alkyl, C₁-C₆alkoxy, C₁-C₆haloalkyl, C₁-C₆haloalkoxy, C₂-C₆alkenyl, C₂-C₆alkynyl, C₃-C₈cycloalkyl, and C₁-C₆alkanoyl.

Further such compounds satisfy Formula IVd or IVe:

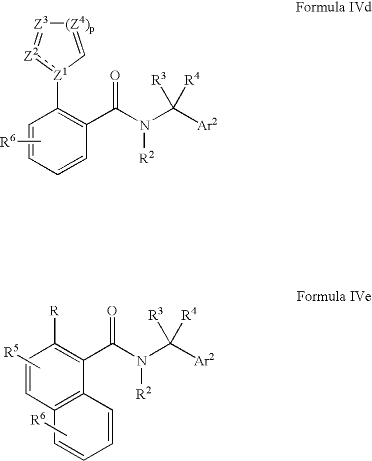

wherein R² is as described for Formula IV; R³, R⁴ and Ar² are as described for Formula IVa; and Z¹, Z² Z³, Z⁴, p, and R⁶ are as described for Formula IVc;

R is: (i) halogen, hydroxy, C₁-C₃haloalkyl, or C₁-C₃haloalkoxy;

(ii) C₁-C₆alkyl, C₂-C₆alkenyl, C₂-C₆alkynyl, C₁-C₆alkoxy, mono- or di-(C₁-C₆alkyl)amino, or C₃-C₇cycloalkyl(C₀-C₄alkyl), each of which is substituted with from 0 to 4 substituents independently chosen from halogen, hydroxy, amino, oxo, cyano, C₁-C₄alkyl, C₁-C₄alkoxy, C₁-C₂haloalkyl, C₁-C₂haloalkoxy, and mono- and di(C₁-C₄)alkylamino;

(iii) phenyl;

(iv) a heterocyclic ring, having from 4 to 8 ring atoms, and 1 to 3 heteroatoms independently selected from N, O, and S;

wherein each of (iii) and (iv) is substituted with from 0 to 3 substituents independently chosen from hydroxy, halogen, amino, cyano, nitro, C₁-C₆alkyl, C₁-C₆alkoxy, mono- and di-C₁-C₆alkylamino, mono- and di-C₁-C₆alkylaminoC₁-C₆alkyl, C₁-C₆haloalkyl, C₁-C₆haloalkoxy, C₁-C₂ alkylthio, and —NHC(═O)C₁-C₂ alkyl; and

R⁵ and R⁶ each represent 0 or more substituents independently chosen from halogen, hydroxy, cyano, nitro, amino, C₁-C₆alkyl, C₂-C₆alkenyl, C₁-C₆alkynyl, C₁-C₆alkoxy, mono- and di-(C₁-C₆)alkylamino, C₁-C₃haloalkyl, C₁-C₃haloalkoxy, C₁-C₂alkylthio, and —NHC(═O)C₁-C₂ alkyl.

Other compounds and salts for use in the combinations provided herein are compounds of Formula V:

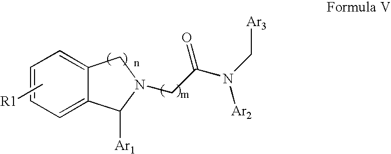

and the pharmaceutically acceptable salts thereof.

In Formula V,

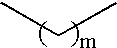

represents a carbon chain that may be substituted with hydrogen, halogen, cyano, nitro amino, mono or dialkyl amino, alkenyl, alkynyl, alkoxy, trifluoromethyl, trifluoromethoxy, straight or branched chain alkyl, or cycloalkyl. and n is 1, 2, or 3.

Ar₁, Ar₂, and Ar₃ are independently optionally substituted carbocyclic aryl, optionally substituted arylalkyl, optionally substituted heteroaryl or heteroalicyclic group having from 1 to 3 rings, 3 to 8 members in each ring and from 1 to 3 heteroatoms.

R₁ represents up to 4 groups independently chosen from hydrogen, halogen, hydroxy, amino, alkoxy, acetoxy, mono- or dialkylamino, cyano, nitro, haloalkyl, alkyl, alkenyl, alkynyl, cycloalkyl, (cycloalkyl)alkyl, hydroxy carbonyl (COOH), aminocarbonyl (CONH₂), mono or dialkylaminocarbonyl, sulfonamido, and mono or dialkylsulfonamido.

The invention includes combinations in which the C5a receptor is a compound of Formula VI:

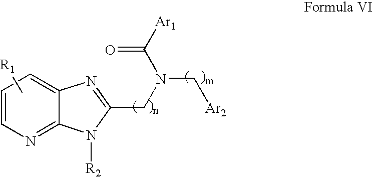

or a pharmaceutically acceptable salt, prodrug or hydrate thereof.

In Formula VI, m is 0, 1, 2, or 3, and

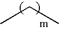

represents a carbon chain which is optionally substituted with methyl, ethyl, methoxy, ethoxy, hydroxy, halogen, or amino; n is 0, 1, 2, or 3, and

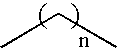

represents a carbon chain which is optionally substituted with methyl, ethyl, methoxy, ethoxy, hydroxy, halogen, or amino.

R₁ represents up to 4 groups independently chosen from hydrogen, halogen, hydroxy, amino, alkoxy, acetoxy, mono- or dialkylamino, cyano, nitro, haloalkyl, alkyl, alkenyl, alkynyl, cycloalkyl, and(cycloalkyl)alkyl.

R₂ is i) hydrogen, halogen, hydroxy, amino, alkoxy, mono- or dialkylamino, cyano, nitro, haloalkyl, and ii) alkyl, alkenyl, alkynyl, cycloalkyl, and (cycloalkyl) alkyl, each of which may be unsubstituted or substituted by one or more of halogen, nitro, cyano, trifluoromethyl, trifluoromethoxy, haloalkyl, hydroxy, acetoxy, alkoxy, amino, mono- or dialkylamino.

Ar₁ is ethylenedioxyphenyl, methylenedioxyphenyl, or optionally substituted carbocyclic aryl, optionally substituted arylalkyl, optionally substituted heteroaryl, optionally substituted heteroarylalkyl, optionally substituted heteroarylalkyl, or an optionally substituted heteroalicyclic or heteroalicyclicalkyl group having from 1 to 3 rings, 3 to 8 members in each ring and from 1 to 3 heteroatoms.

Ar₂ is carbocyclic aryl, optionally substituted arylalkyl, optionally substituted heteroaryl, optionally substituted heteroarylalkyl, optionally substituted heteroarylalkyl, or an optionally substituted heteroalicyclic or heteroalicyclicalkyl group having from 1 to 3 rings, 3 to 8 members in each ring and from 1 to 3 heteroatoms.

Preferred compounds for use in the combinations provided herein exhibit an IC₅₀ of about 500 nM or less in such a standard C5a receptor-mediated chemotaxis, radioligand binding, and/or calcium mobilization assay, more preferably an IC₅₀ of about 250 nM or less in such an assay, still more preferably an IC₅₀ of about 200, 150, 100, 50, 25, 10, or 5 nM or less in such an assay. Such assays are provided in Examples 5, 7 and 8.

Certain preferred compounds further have favorable pharmacological properties, including oral bioavailability (such that a sub-lethal or preferably a pharmaceutically acceptable oral dose, preferably less than 2 grams, more preferably of less than or equal to one gram, can provide a detectable in vivo effect such as a reduction of C5a-induced neutropenia), ability to inhibit leukocyte chemotaxis at nanomolar concentrations and preferably at sub-nanomolar concentrations, low toxicity (a preferred compound is nontoxic when a C5a receptor-modulatory amount is administered to a subject), minimal side effects (a preferred compound produces side effects comparable to placebo when a C5a receptor-modulatory amount of the compound is administered to a subject), low serum protein binding, and a suitable in vitro and in vivo half-life (a preferred compound exhibits an in vitro half-life that is equal to an in vivo half-life allowing for Q.I.D. dosing, preferably T.I.D. dosing, more preferably B.I.D. dosing, and most preferably once-a-day dosing). Distribution in the body to sites of complement activity is also desirable (e.g., compounds used to treat CNS disorders will preferably penetrate the blood brain barrier, while low brain levels of compounds used to treat periphereal disorders are typically preferred).

Routine assays that are well known in the art may be used to assess these properties, and identify superior compounds for a particular use. For example, assays used to predict bioavailability include transport across human intestinal cell monolayers, such as Caco-2 cell monolayers. Penetration of the blood brain barrier of a compound in humans may be predicted from the brain levels of the compound in laboratory animals given the compound (e.g., intravenously). Serum protein binding may be predicted from albumin binding assays, such as those described by Oravcová, et al. (1996) Journal of Chromatography B 677:1-27. Compound half-life is inversely proportional to the frequency of dosage of a compound required to achieve an effective amount. In vitro half-lives of compounds may be predicted from assays of microsomal half-life as described by Kuhnz and Gieschen (1998) Drug Metabolism and Disposition 26:1120-27.

Toxicity and side effects may be assessed using any standard method. In general, the term “nontoxic” as used herein shall be understood in a relative sense and is intended to refer to any substance that has been approved by the United States Food and Drug Administration (“FDA”) for administration to mammals (preferably humans) or, in keeping with established criteria, is susceptible to approval by the FDA for administration to mammals (preferably humans). Toxicity may be also evaluated using the assay detecting an effect on cellular ATP production. Other assays that may be used include bacterial reverse mutation assays, such as an Ames test, as well as standard teratogenicity and tumorogenicity assays. Preferably, administration of compounds provided herein at certain doses (i.e., doses yielding effective in vivo concentrations) does not result in prolongation of heart QT intervals (i.e., as determined by electrocardiography in guinea pigs, minipigs or dogs). When administered daily for five or preferably ten days, such doses also do not cause liver enlargement resulting in an increase of liver to body weight ratio of more than 100%, preferably not more than 75%, and more preferably not more than 50% over matched controls in laboratory rodents (e.g., mice or rats). Such doses also preferably do not cause liver enlargement resulting in an increase of liver to body weight ratio of more than 50%, preferably not more than 25%, and more preferably not more than 10% over matched untreated controls in dogs or other non-rodent mammals.

Certain preferred compounds also do not promote substantial release of liver enzymes (e.g., ALT, LDH or AST) from hepatocytes in vivo. Preferably the above doses do not elevate serum levels of such enzymes by more than 100%, preferably not by more than 75%, and more preferably not by more than 50% over matched untreated controls in vivo in laboratory rodents. Similarly, concentrations (in culture media or other such solutions that are contacted and incubated with cells in vitro) equivalent to two-fold, preferably five-fold, and most preferably ten-fold the minimum in vivo therapeutic concentration do not cause detectable release of any of such liver enzymes from hepatocytes in vitro into culture medium above baseline levels seen in media from untreated cells.

In certain embodiments, preferred compounds exert their receptor-modulatory effects with high specificity. This means that they only bind to, activate, or inhibit the activity of certain receptors other than C5a receptors with affinity constants of greater than 100 nanomolar, preferably greater than 1 micromolar, more preferably greater than 4 micromolar. The invention also includes highly specific C5a receptor modulatory compounds that exhibit 200-fold greater affinity for the C5a receptor that for other cellular receptors. Such receptors include neurotransmitter receptors such as alpha- or beta-adrenergic receptors, muscarinic receptors (particularly m1, m2 or m3 receptors), dopamine receptors, and metabotropic glutamate receptors; as well as histamine receptors and cytokine receptors (e.g., interleukin receptors, particularly IL-8 receptors). Such receptors may also include GABA_{A} receptors, bioactive peptide receptors (other than C5a receptors and C3a receptors, including NPY or VIP receptors), neurokinin receptors, bradykinin receptors, and hormone receptors (e.g., CRF receptors, thyrotropin releasing hormone receptors or melanin-concentrating hormone receptors). Compounds that act with high specificity generally exhibit fewer undesirable side effects.

Within certain embodiments, modulators provided herein do not bind detectably to receptors that do not mediate inflammatory responses, such as GABA receptors, MCH receptors, NPY receptors, dopamine receptors, serotonin receptors and VR1 receptors, with high or even moderate affinity. In addition, or alternatively, certain preferred C5a receptor modulators exhibit an affinity for C5a receptor that is substantially higher than for receptors that do not mediate inflammatory responses (e.g., at least five times higher, at least ten times higher or at least 100 times higher). Assays for evaluating binding to receptors that do not mediate inflammatory responses include, for example, those described in U.S. Pat. No. 6,310,212, which is incorporated herein by reference for its disclosure of a GABA_{A} receptor binding assays in Examples 14, columns 16-17, in U.S. patent application Ser. No. 10/152,189 which is incorporated herein by reference for its disclosure of an MCH receptor binding assay in Example 2, pages 104-105, in U.S. Pat. No. 6,362,186, which is incorporated herein by reference for its disclosure of CRF1 and NPY receptor binding assays in Examples 19, columns 45-46, in U.S. Pat. No. 6,355,644, which is incorporated herein by reference for its disclosure of a dopamine receptor binding assay at column 10, and in U.S. Pat. No. 6,482,611, which is incorporated herein by reference for its disclosure of VR1 receptor binding assays in Examples 4-5, column 14. It will be apparent that the C5a receptor modulators provided herein may, but need not, bind to one or more other receptors known to mediate inflammatory responses, such as C3a receptors and/or A₃ receptors.

Certain preferred compounds are C5a receptor antagonists that do not possess significant (e.g., greater than 5%) agonist activity in any of the C5a receptor-mediated functional assays discussed herein. Specifically, this undesired agonist activity can be evaluated, for example, in the GTP binding assay of Example 6, by measuring small molecule mediated GTP binding in the absence of the natural agonist, C5a. Similarly, in a calcium mobilization assay (e.g., that of Example 6) a small molecule compound can be directly assayed for the ability of the compound to stimulate calcium levels in the absence of the natural agonist, C5a. The preferred extent of C5a agonist activity exhibited by compounds provided herein is less than 10%, 5% or 2% of the response elicited by the natural agonist, C5a.

Additionally, preferred C5a receptor modulators do not inhibit or induce microsomal cytochrome P450 enzyme activities, such as CYP1A2 activity, CYP2A6 activity, CYP2C9 activity, CYP2C19 activity, CYP2D6 activity, CYP2E1 activity or CYP3A4 activity. Preferred C5a receptor modulators also do not exhibit cytotoxicity in vitro or in vivo, are not clastogenic (e.g., as determined using a mouse erythrocyte precursor cell micronucleus assay, an Ames micronucleus assay, a spiral micronucleus assay or the like) and do not induce sister chromatid exchange (e.g., in Chinese hamster ovary cells). Also preferred are C5a receptor modulators that inhibit the occurrence of C5a-induced oxidative burst (OB) in inflammatory cells (e.g., neutrophil) as can be conveniently determined using an in vitro neutrophil OB assay.

Combination Therapy

Within the therapeutic methods provided herein, at least one therapeutic compound that is not a C5a antagonist (i.e., a C5a receptor-inactive therapeutic agent) is administered in combination with at least one C5a antagonist in order to provide a therapeutic effect to a patient suffering from a condition (e.g., disease, disorder or injury) associated with pathogenic inflammation. The C5a antagonist(s) and C5a receptor-inactive agent(s) may be present in the same composition during administration, or may be administered separately, either essentially simultaneously or sequentially in either order. In certain embodiments, the C5a receptor-inactive therapeutic agent(s) are packaged in combination with the C5a antagonist(s). The precise formulation of combinations described herein will vary depending on the disease to be treated, as discussed in more detail below.

Combinations for the Treatment of Arthritis

In one aspect, the present invention provides combinations useful for the treatment of arthritis, particularly rheumatoid arthritis, comprising a C5a antagonist and a C5a receptor-inactive therapeutic agent that is an anti-arthritic agent (i.e., a C5a receptor-inactive anti-arthritic agent). C5a receptor-inactive therapeutic agents useful in such combinations include, but are not limited to NSAIDs, non-specific and COX-2 specific cyclooxgenase enzyme inhibitors, gold compounds, corticosteroids, methotrexate, tumor necrosis factor receptor (TNF) receptors antagonists, immunosuppressants and modulators of other enzymes and receptors associated with arthritis. In one embodiment, a C5 antagonist is combined with methotrexate for treatment of rheumatoid arthritis.

In one embodiment, the C5a receptor-inactive therapeutic agent is a non-steroidal anti-inflammatory drug (NSAID). Examples of NSAIDs include, but are not limited to ibuprofen (e.g., ADVIL™, MOTRIN™), flurbiprofen (ANSAID™), naproxen or naproxen sodium (e.g., NAPROXYN, ANAPROX, ALEVE™), diclofenac (e.g., CATAFLAM™, VOLTAREN™), combinations of diclofenac sodium and misoprostol (e.g., ARTHROTEC™), sulindac (CLINORIL™), oxaprozin (DAYPRO™), diflunisal (DOLOBID™), piroxicam (FELDENE™), indomethacin (INDOCIN™), etodolac (LODINE™), fenoprofen calcium (NALFON™), ketoprofen (e.g., ORUDIS™, ORUVAIL™), sodium nabumetone (RELAFEN™), sulfasalazine (AZULFIDINE™), tolmetin sodium (TOLECTIN™), and hydroxychloroquine (PLAQUENIL™). In some cases, the C5a receptor-inactive therapeutic agent(s) comprise one or more NSAIDS combined with one or more anti-ulcer agents such as misoprostol (CYTOTEC™). Accordingly, the invention comprises the use of a preparation of diclofenac and misoprostol (e.g., as marketed under the brand name ARTHROTEC™) as the C5a receptor-inactive therapeutic agents.

Certain NSAIDs inhibit cyclooxygenase (COX) enzymes. In one embodiment, at least one C5a receptor-inactive therapeutic agent is a COX-2 specific inhibitor (i.e., a compound that inhibits COX-2 with an IC₅₀ that is at least 50-fold lower than the IC₅₀ for COX-1). Preferably, a COX-2 inhibitor such as celecoxib (CELEBREX™), valdecoxib (BEXTRA™), lumiracoxib (PREXIGE™), etoricoxib (ARCOXIA™) and/or rofecoxib (VIOXX™). In a further embodiment, at least one C5a receptor-inactive therapeutic agent is a salicylate. Salicylates include by are not limited to acetylsalicylic acid or aspirin, sodium salicylate, choline and magnesium salicylates (TRILISATE™), and salsalate (DISALCID™). The C5a receptor-inactive therapeutic agent may also be a corticosteroid. For example, the corticosteroid may be cortisone (CORTONE™ acetate), dexamethasone (e.g., DECADRON™), methylprednisolone (MEDROL™) prednisolone (PRELONE™), prednisolone sodium phosphate (PEDIAPRED™), and prednisone (e.g., PREDNICEN-M™, DELTASONE™, STERAPRED™). Additional coriticosteroids are listed herein in the description of combinations for the treatment of asthma.

In further embodiments, at least one C5a receptor-inactive therapeutic agent is a gold compound such as gold sodium thiomalate (MYOCHRYSINE™) or auranofin (RIDAURA™). The invention also includes embodiments in which the C5a receptor-inactive therapeutic agent is a metabolic inhibitor such as a dihydrofolate reductase inhibitor, such as methotrexate (e.g., RHEUMATREX™, TREXALL™) or a dihydroorotate dehydrogenase inhibitor, such as leflunomide (ARAVA™). In other embodiments for the treatment of joint disease, at least one C5a receptor-inactive therapeutic agent is a joint lubricant such as sodium hyaluronate (HYALGAN™ or SYNVISC™). Other embodiments of the invention are directed to combinations in which that least one C5a receptor-inactive therapeutic agent is an immunosuppressant compound such as methotrexate, leflunomide, cyclosporine (NEORAL™, SANDIMMUNE™), tacrolimus (PROGRAF™), azathioprine (IMURAN™), or mycophenolate mofetil (CellCept™).

Still other embodiments of the invention are directed to combinations in which at least one C5a receptor-inactive therapeutic agent is:

an anti-C5 monoclonal antibody (such as eculizumab or pexelizumab);

a TNF antagonist, such as entanercept (ENBREL™), which is an injectible fusion protein consisting of the extracellular domain of the TNF receptor and the Fe portion of human IgG1, adalimumab (MUNIRA™), the humanized antibody CDP-571 (HUMICADE™; WO 92/11383), the anti-TNFα humanized antibody fragment CDP-870 (WO 01/94585), or infliximab (REMICADE™), which is an anti-TNF alpha monoclonal antibody;

a modulator of TNF-alpha induced inflammatory genes, such as AGIX-4207 (AtheroGenics, Inc.);

an antisense oligonucleotide specific for TNFa mRNA such as ISIS 104838 (Isis Pharmaceuticals, Inc.);

a natural product such as RA-1;

an IL-1 receptor antagonist, such as anakinra (KINERET™) or AMG-719 (Amgen);

IL-1 receptor, type I or type II, such as SIL-1r2 (Amgen);

an IL-1 trap, such as Regeneron's IL-1 trap;

an IL-6 receptor antagonist, such as the humanized anti-IL-6 receptor monoclonal antibody Altizumab (WO 92/19759);

an anti IL-12 antibody, such as the human anti-IL-12 monoclonal antibody J695 (Abbott Laboratories);

an IL-15 antagonist such as the human anti-IL-15 monoclonal antibody HuMax IL-15 (Genmab);

a B-cell targeted chimeric monoclonal antibody such as Rituximab (RIUXAN™);

a cytotoxic T lymphocyte antigen 4 immunoglobulin such as CTLA4Ig (BMS-188667; EP 00613944);

an inhibitor of p38 mitogen-activated protein (MAP) kinase such as AMG-548, BIRB-796, VX-702, VX-850, VX-745, SCIO-469, SCIO-323, or GXK-681323;

an integrin antagonist such as GSK-683699;

a TNF alpha converting enzyme (TACE) inhibitor such as BMS-561392;

an inhibitor of IKappaB kinase (IKK), phospholipase A2 or an LCK-selective tyrosine kinase;

a CCR3 receptor antagonist such as DPC-168 or GXK-766994;

an ICE inhibitor such as VX-740.

Combinations for the Treatment of Lung Disorders

Certain compositions and methods provided herein are useful for the treatment of respiratory diseases, such as respiratory distress syndrome and asthma. For asthma therapy, combinations provided herein may be used to prevent or decrease the severity of both acute early phase asthma attack and the late phase reactions that follow such an asthma attack. C5a receptor-inactive therapeutic agents useful for the treatment of asthma include anti-thrombin agents, which reduce bronchoconstriction by inhibiting the release of calcium and proliferation of smooth muscle cells, adrenergic receptor agonists, particularly Beta adrenergic receptor agonists; methylxanthines such as theophylline (e.g., AEROLATE SR™, AEROLATE JR™, THEO-DUR™, UNI-DUR™, or UNIPHYL™); corticosteroids; leukotriene modifiers such as zafirlukast (e.g., ACCOLATE™) and zileuton (e.g., ZYFLO™, or FILMTAB™); anticholinergics such as ipratropium bromide (e.g., ATROVENT™, COMBIVENT™, DUONEB™); phosphodiesterase 4 (PDE4) inhibitors such as cilomilast (ARIFLO™); and cromolyn sodium (e.g., NASALCROM™), which act by stabilizing mast cells and reducing the release of cellular mediators.

Representative corticosteroids include, but are not limited to betamethasone (e.g., Celestone™), beclomethasone dipropionate (e.g., VANCERIL™, QVAR™), budesonide (e.g., PULMICORT™), cortisone (e.g., CORTONE ACETATE™), dexamethasone (e.g., DEXASONE™), fluticasone propionate, (e.g., ADVAIR™, FLOVENT™) hydrocortisone, ethylprednisolone (e.g., MEDROL™), flunisolide (AEROBID™), prednisolone (e.g., PREDALONE™, PRELONE™), prednisone (e.g., CORDROL™, DELTASONE™, STERAPRED™, STERAPRED DS™), and triamcinolone (AZMACORT™). Additional coriticosteroids are listed herein in the description of combinations for the treatment of arthritis.

Preferred Beta adrenergic receptor agonists are Beta-2 adrenergic receptor agonists. The Beta agonist may be a long or short acting Beta adrenergic receptor agonist. Examples of Beta adrenergic receptor agonists include, but are not limited to, albuterol (e.g., PROVENTIL™, VENTELIN™), bitolterol, epinephrine (e.g., PRIMATENE™, ADRENALIN CHLORIDE, BRONCAID MIST), fenoterol (e.g., FORADIL™), formoterol, isoetharine, isoproterenol, metaproterenol sulfate (e.g., ALUPENT™), pirbuterol (e.g., MAXAIR), procaterol, racepinephrine, salmeterol xinafoate (e.g., ADVAIR DISKUS™, SEREVENT DISKUS™) and terbutaline (e.g., BRETHINE™).

Combinations for the Treatment of Skin Injury and Disease

The present invention further provides combinations useful for the treatment of T cell-mediated inflammatory diseases such as psoriasis. The C5a receptor-inactive therapeutic agent(s) may inhibit the hyperfunctional proliferation of epidermal cells; inhibit the inflammatory response; promote immunomodulation; and/or inhibit infection by bacteria and fungi. Included in the invention are combinations in which the C5a receptor-inactive therapeutic agent is a non-steroidal anti-inflammatory drug (NSAID), such as the non-specific nonsteroidal anti-inflammatory agents, COX-2 specific and salicylates listed above.

The present invention includes combinations in which the C5a receptor-inactive therapeutic agent is a steroid (e.g., corticosteroid such as clobetasol propionate (e.g., TEMOVATE™), coal tar, a moisturizer, calendula plant extract, theophylline (which arrests the proliferation of cells during the metaphase stage of cell division; e.g., SLO-PHYLLIN™ or THEO-DUR SPRINKLE™), anthralin (a synthetic derivative of a tree bark extract; e.g., DRITHOCREME™, DRITHO-SCALP™ or MICANOL™), calcipotriene (a synthetic vitamin D-3 analog that regulates skin cell production; e.g., CALCIOPTRIOL™ or DOVONEX™), dithranol, an angiogenesis inhibitor such as ganglioside GM3 or a GM3 analog, a phospholipase A2 inhibitor such as an n-deacetyl-lysoganglioside derivative, hydroxyurea, sulfasalazine, 6-thioguanine, betamethasone valerate, mycophenolate mofetil, parathyroid hormone or an imidazole antifungal.

Further combinations comprise a C5a receptor-inactive therapeutic agent that is an immunomodulator. Such agents may be included to control the abnormal cornification of epidermal cells and the hyperfunction of leukocyte migration, and include methotrexate (a folic acid antagonist that inhibits DNA synthesis in tissues with high rates of turnover and is immunosuppressive to mononuclear cells), cyclosporine (which inhibits production of interleukin-2, the cytokine responsible for inducing T-cell proliferation), tacrolimus (FK506; e.g., PROGRAF™ or PROTOPIC™), TNF-alpha modulators such as etanercept (e.g., ENBREL™) and infliximab (e.g., REMICADE™), antileukotriene agents, and retinoid Vitamin A derivatives such as tazarotene (e.g., TAZORAC™), etretinate, isotretinoin (e.g., ACCUTANE™), bexarotene, DAB(389)IL-2 (e.g., DENILEUKIN DIFTITOX™) or acitretin (e.g., SORIATANE™).

Combinations provided herein may also be used in the treatment of other skin conditions associated with inflammation, including bullous pemphigoid and lichen planas. Within such compositions, the C5a receptor-inactive agent may be, for example, a steroid such as prednisone, an antibiotic such as tetracycline or dapsone, an immunosuppressant such as azothioprine, or methotrexate.

Still further combinations are useful for promoting healing of burns and wounds. Such compositions are generally formulated for topical administration to a wound or burn site. Any agent that facilitates healing of such conditions may be used as the C5a receptor-inactive therapeutic agent(s) including, for example, antibiotics and growth factors.

Combinations for the Treatment of Other Autoimmune Disorders

The present invention also provides combinations useful for treating autoimmune disorders and pathologic autoimmune responses known to have an inflammatory component including, but not limited to multiple sclerosis, myasthenia gravis, Alzheimer's disease, glomerulonephritis, Crohn's disease, Guillain-Barre Syndrome, lupus erythematosus and irritable bowel syndrome. A C5a receptor-antagonist may be combined with another anti-inflammatory agent to increase the effectiveness the anti-inflammatory agent, or may be combined with a C5a receptor-inactive therapeutic agent suitable for the disease of interest that is not an anti-inflammatory agent. Suitable C5a receptor-inactive therapeutic agents include, for example, steroids as discussed above.

Combinations for the Treatment of Infection

Also provided herein are combinations for use in treating sepsis and other potentially life-threatening infections, including drug-resistant bacterial infections, such as pneumococcal bacteremia, pneumococcal meningitis, nosocomial (hospital-acquired) infections (e.g., Staphylococcus aureus and Enterococcus faecium), Group A streptococci infections, multiple drug-resistant tuberculosis, and infections with other bacteria such as Acinetobacter spp., Enterobacter spp., E. coli, H. influenzae, K. pneumoniae, P. aeruginosa, S. marcescens, S. maltophilia, Bordetella pertussis, Brucella spp., Campylobacter spp., Haemophilus ducreyi, Helicobacter pylori, Legionella spp., Moraxella catarrhalis, Neisseria spp., Salmonella spp., Shigella spp., Yersinia spp., Bacillus spp., E. faecalis, S. pyogenes, and coagulase-negative Staphylococci, Corynebacterium spp., Diphtheroids, Listeria spp., and Viridans Streptococci. Suitable C5a receptor-inactive therapeutic agents for use in such combinations include, for example, antibiotic agents. Representative anti-bacterial antibiotic agents include, for example, penicillins, cephalosporins, carbacephems, cephamycins, carbapenems, monobactams, aminoglycosides, glycopeptides (e.g., vancomycin), quinolones, tetracyclines, macrolides, and fluoroquinolones. Combinations may be administered after a patient has acquired an infection, or may be administered to a patient considered at risk for such an infection (e.g., post-surgically).

Combinations for use in Medical Procedures

Certain combinations are provided herein are suitable for use in medical procedures in which foreign material is introduced into a patient. For example, such combinations may be used to prevent or decrease rejection of transplanted organs and tissue grafts, or in hemodialysis or cardiopulmonary bypass surgery. Certain compositions may be used in the treatment of lung inflammation associated with transplantation. C5a receptor-antagonists may be combined with another anti-inflammatory agent to increase the effectiveness the anti-inflammatory agent or combined with a C5a receptor-inactive therapeutic agent that is not an anti-inflammatory agent in order to decrease complications and improve outcome for the patient undergoing the medical procedure.

Combinations may also be used to prevent activation of platelets during storage, and thereby decreasing the loss of function and viability known as “platelet storage lesion.” Such combinations are typically added to platelets prior to storage.

Combinations for the Treatment of Cardio- and Cerebrovascular Disease

Also provided herein are combinations useful for the treatment of cardio- and cerebrovascular disease and reducing the risk of cardio- and cerebrovascular events including myocardial infarction and stroke. Combinations provided herein are useful for preventing or reducing the risk of formation of thrombi and thromboemboli, for preventing or reducing the risk of thrombotic occlusions and reocclusions, for treating, preventing or reducing the risk of a first or subsequent myocardial infarction, for preventing or reducing the risk of restenosis, for treating, preventing or reducing the risk of acute cerebrovascular ischemic events such as a first or subsequent thrombotic stroke or transient ischemic attack, and for halting or slowing the progression of atherosclerotic disease. C5a receptor-inactive therapeutic agents useful in the combinations provided herein for the treatment or cardio- and cerebrovascular disease include, but are not limited to, anti-inflammatories (e.g., non-steroidal anti-inflammatory drugs (NSAIDs) such as the non-specific nonsteroidal anti-inflammatory agents, COX-2 specific and salicylates listed above), antihypertensive agents, platelet inhibitors such as aspirin, ticlopidine, and GP IIb/IIIa antagonists, antihypertensive agents, including adrenergic receptor agonists, alpha/beta adrenergic receptor antagonists, beta adrenergic receptor antagonists, angiotensin converting enzyme (ACE) inhibitors, angiotensin II receptor antagonists, calcium channel blockers, diuretics and periphereal vasodilators, anti-coagulants, thrombolytics, and cholesterol lowering agents such as HMG-CoA reductase inhibitors.

Within certain combinations, the C5a receptor-inactive therapeutic agent is a cholesterol lowering drug. In one embodiment the cholesterol lowering drug is a 3-hydroxy-methyl-glutaryl coenzyme A reductase inhibitor (HMG-CoA RI). Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33.

Examples of HMG-CoA reductase inhibitors that may be used include but are not limited to lovastatin (MEVACOR™; see U.S. Pat. No. 4,231,938), simvastatin (ZOCOR™; see U.S. Pat. No. 4,444,784), pravastatin (PRAVACHOL™; see U.S. Pat. No. 4,346,227), fluvastatin (LESCOL™; see U.S. Pat. No. 5,354,772), atorvastatin (LIPITOR™.; see U.S. Pat. No. 6,273,995) and cerivastatin (also known as rivastatin; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (Feb. 5, 1996). The term HMG-CoA reductase inhibitor is intended to include all pharmaceutically acceptable salt, ester and lactone forms of compounds which have HMG-CoA reductase inhibitory activity, and therefore the use of such salts, esters and lactone forms is included within the scope of this invention.

In further combinations, the C5a receptor-inactive therapeutic agent is a platelet aggregation inhibitor such as a salicylate as described above; a glycoprotein (GP) IIb/IIIa inhibitor, such as tirofiban hydrochloride (AGGRASTAT™), abciximab (REOPRO™), or eptifibatide (INTEGRILIN™); a phosphodiesterase (PDE) inhibitor, such as dipyridimole (PERSANTINE™) or cilostazol (PLETAL™); a compound that reduces megakaryocyte hypermaturation, (e.g., anagrelide, AGRYLIN™); or ADP receptor inhibitor such as clopidogrel bisulfate (PLAVIX™) or ticlopidine hydrochloride (TICLID™).

Within other combinations, the C5a receptor-inactive therapeutic agent is an anti-hypertensive agent. In one such embodiment, the anti-hypertensive agent is an alpha adrenergic receptor antagonist such as phenoxybenzamine hydrochloride (DIBENZYLINE™), doxazosin mesylate (CARDURA™), terazosin hydrochloride (HYTRIN™), or prazosin hydrochloride (MINIPRESS™).

In other embodiments, the antihypertensive agent is an adrenergic receptor stimulator. Such antihypertensive agents may affect adrenergic receptors either directly (adrenergic receptor agonists) or through interaction with a separate receptor or enzyme that affects the activity of adrenergic receptors. Thus, adrenergic receptor stimulators that may be used in the combinations provided herein include, but are not limited to, methyldopa (ALDOMET™), clonidine hydrochloride (CATAPRES™), and guanfacine hydrochloride (TENEX™).

In still further embodiments, the antihypertensive agent is a non-specific alpha/beta adrenergic receptor antagonist such as carvedilol (COREG™) or labetalol hydrochloride (NORMODYNE™).

Within other such embodiments, the antihypertensive agent is an angiotensin converting enzyme (ACE) inhibitor. Suitable angiotensin converting enzyme (ACE) inhibitors include, but are not limited to enalaprilat (VASOTEC™ for injection), enalapril maleate (VASOTEC™), catopril, benazepril hydrochloride (LOTREL™, LOTENSIN™), fosinopril sodium (MONOPRIL™), lisinopril (PRINUVIL™ and ZESTRIL™), quinapril (ACCUPRIL™), perindopril erbumine (ACEON™), ramipril (ACEON™), moexipril hydrochloride (UNIVASC™), and trandolapril (MAVIK™).

In other embodiments, the antihypertensive agent is an angiotensin II receptor antagonist such as candesartan cilexetil (ATACAND™), irbesartan (AVAPRO™), losartan potassium (COZAAR™), valsartan (DIOVAN™), telmisartan (MICARDIS™), or eprosartan mesylate (TEVETEN™).

Within further such embodiments, the antihypertensive agent is a beta-adrenergic receptor blocker, including combinations in which the antihypertensive agent is a beta-adrenergic receptor antagonist. Beta-adrenergic receptor blockers include, but are not limited to chlorthalidone, sotalol hydrochloride (BETAPACE™ and BETAPACE AF™), timolol maleate (BLOCADREN™), nadolol (CORGARD™), propranolol hydrochloride (INDERAL™), acebutolol hydrochloride (SECTRAL™), atenolol (TENORMIN™), metoprolol succinate (TOPROL-XL™), and bisoprolol fumarate (ZEBETA™).

In other embodiments, the antihypertensive agent is a calcium channel blocker, such as felodipine (PLENDIL™), nifedipine (ADALAT™, PROCARDIA™, PROCARDIA™), nicardipine hydrochloride (CARDENE™), nimodipine (NIMOTOP™), amlodipine besylate (NORVASC™) diltiazem hydrochloride (CARDIZEM™), verapamil hydrochloride (COVERA-HS™, ISOPTIN™, VERELAN™, VERELAN PM™), isradipine (DYNACIRC™, DYNACIRC CR™), nisoldipine (SULAR™), diltiazem hydrochloride (TIAZIC™), and bepridil hydrochloride(VASCOR™).

Also provided herein are combinations in which the C5a receptor-inactive therapeutic agent is a diuretic such as ionized potassium, chlorothiazide (DIURIL™), chlorthalidone, hydrochlorothiazide (HYDRODIURIL™, MICROZIDE™), polythiazide (RENESE™), bendroflumethiazide, atenolol, dichlorphenamide (DARANIDE™), torsemide (DEMADEX™), ethacrynic acid (EDECRIN™), furosemide, triamterene (DYRENIUM™) amioride (MIDAMOR™), hydroflumethiazide (DIUCARDIN™), indapamide, or metolazone (MYKROX™, ZAROXOLYN™).

In other aspects, the C5a receptor-inactive therapeutic agent is blood modifier, such as an anticoagulant. Anticoagulants useful in the combinations provided herein include, but are not limited to, thrombin inhibitors such as argatroban, inhibitors of thrombin and Factor X, such as dalteparin sodium injection (FRAGMIN™), heparin (e.g., TUBEX™ Heparin Sodium Injection, INNOHEP™), enoxaparin sodium a low molecular weight heparin (LOVENOX™), danaparoid sodium (ORGARAN™) inhibitors of vitamin K dependent coagulation, such as coumarin and warfarin, and hirudin.

The present invention provides, in an additional embodiment, a combination comprising a C5a antagonist and a C5a receptor-inactive therapeutic agent that is a thrombolytic. Examples of thrombolytics include, but are not limited to recombinant alteplase (ACTIVASE™), anistreplase (EMINASE™), recombinant reteplase (RETAVASE™), streptokinase (STREPTASE™), and urokinase (ABBOKINASE™).

Also provided herein are combinations in which the C5a receptor-inactive therapeutic agent is a prescribed combination, such as AGGRENOX™, a tablet comprised of dypridimole and aspirin or MINIZIDE™, a marketed combination of polythiazide (RENESE™) and prazosin hydrochloride (MINIPRESS™), MICARDIS™ HCT (telmisartan and hydrochlorothiazide), ATACAND™ HCT (candesartan cilexetil and hydrochlorothiazide), AVALIDE™ (irbesartan and hydrochlorothiazide), DIOVAN™ HCT (valsartan and hydrochlorothiazide), HYZAAR™ (losartan potassium-hydrochlorothiazide tablets), CORZIDE™ (nadolol and bendroflumethiazide), INDERIDE™ (propranolol hydrochloride and hydrochlorothiazide), TENORETIC™ (atenolol and chlorthalidone), TIMOLIDE™ (timolol maleate and hydrochlorothiazide), ZIAC™ (bisoprolol fumarate and hydrochlorothiazide), CLORPRES™ and COMBIPRES™, both of which are marketed combinations of clonidine hydrochloride and chlorthalidone, LEXXEL™, a combination of enalapril and felodipine, ALDOCLOR™, a tablet containing methyldopa and chlorothiazide, TAREX™, a combination of trandolapril and verapamil hydrochloride, and ALDORIL™ (methyldopa and hydrochlorothiazide).

Combinations for the Treatment of Ischemia-Reperfusion Injury

Certain combinations provided herein are useful for treating or preventing reperfusion injury due to thrombosis or surgery. The invention particularly provides combinations for preventing or reducing reperfusion injury during surgeries in which blood vessels are occluded, or in which a heart bypass pump is employed. The C5a receptor-inactive agent(s) used in the combination may be one or more or an anticoagulant or thrombolyitc agent, as described above in the discussion of combinations for the treatment of cardio- and cerebrovascular disease, and/or a surgical anesthetic.

Combinations for the Treatment of Traumatic Injury, Infection or Organ Failure

The present invention includes combinations useful for the treatment of CNS trauma (e.g., injury to the head or spinal cord). Injuries to the brain and spinal cord generally result in an upregulation of pro-inflammatory cytokines and subsequent leukocyte recruitment leading to tissue destruction beyond the original site of injury. The C5a receptor-inactive therapeutic agent(s) may further inhibit such inflammation, or may mediate pain or damage resulting form other aspects of CNS trauma. Suitable anti-inflammatory agents include those described elsewhere herein (e.g., steroids such as methylprednisone), as well as agents (e.g., antibodies) that inhibit proinflammatory cytokine responses, such as IL-1, IL-6, IL-8 and TNF. Other suitable C5a receptor-inactive therapeutic agents include, for example, diazepam (to control seizures). Further therapies that may be used in combination with the C5a receptor antagonist are surgery to control bleeding and ensure adequate blood flow to the brain, as well as therapies that promote nerve growth, reduce scar tissue barriers, repair damaged myelin and promote compensatory growth of intact nerve fibers. Combinations provided herein may also be used in the treatment of hemorrhagic shock, multiple organ system failure or sepsis.

Dosage Information

Therapeutically effective amounts of C5a antagonist(s) and C5a receptor-inactive therapeutic agent(s) are preferred for use in the compositions and methods of the present invention. Dosages and methods of administration of therapeutic agents that are not C5a receptor antagonists are known to those skilled in the medical and pharmaceutical arts and can be found, for example, in the manufacturer's instructions set forth in the package insert for the agent, conveniently recorded in the Physician's Desk Reference. In determining the optimal mode of administration, it will be apparent that is necessary to take into account that certain therapeutic compounds, such as NSAIDS, are suitable for oral administration while others, such as antibody-based drugs, are typically only suitable for non-oral administration.

In certain particularly preferred embodiments, the combination administration of at least one C5a receptor antagonist with at least one C5a receptor-inactive therapeutic agent results in a reduction of the dosage of the C5a receptor-inactive therapeutic agent required to produce a therapeutic effect. Thus, preferably, the dosage of a C5a receptor-inactive therapeutic agent in a combination or combination treatment method of the invention is less than the maximum dose advised by the manufacturer for administration of the C5a receptor-inactive therapeutic agent without combination administration of a C5a receptor antagonist. More preferably this dosage is less than ¾, even more preferably less than ½, and highly preferably, less than ¼ of the maximum dose, while most preferably the dose is less than 10% of the maximum dose advised by the manufacturer for administration of the C5a receptor-inactive therapeutic agent(s) when administered without combination administration of a C5a receptor antagonist. It will be apparent that the dosage amount of C5a antagonist component of the combination needed to achieve the desired effect may similarly be affected by the dosage amount and potency of the C5a receptor-inactive therapeutic agent component of the combination.

For compounds administered orally, transdermally, intravaneously, or subcutaneously, dosage of a C5a receptor antagonist administered as part of a combination in as described herein is preferably adjusted to achieve specific levels of the C5a receptor antagonist in a body fluid that is in contact with the site of disease or injury (a target body fluid) in the patient being treated. Such target body fluids include, for example one or more of 1) in arthritis, synovial fluid, 2) in, e.g., septicemia, myocardial infarction and conditions involving a potential for reperfusion injury, blood, plasma, or preferably serum, 3) in, e.g., traumatic spinal or brain injury, cerebrospinal fluid, 4) in, e.g., retinopathy, aqueous humor, and 5) in various conditions including the foregoing, cellular interstitial fluid or lymphatic fluid. When administered as part of a combination therapy (e.g., by intravenous, intra-articular or intrathecal injection), preferred levels to be achieved in a target body fluid range from about 5 ng/mL to about 10 ug/mL, preferably from about 20 ng/mL to about 1 ug/mL, more preferably from about 30 ng/mL to about 500 ng/ml, and most preferably from about 50 ng/ml to about 100 ng/ml. Dosages and routes of administration are preferably adjusted to achieve such preferred levels.

Oral dosage amount of the C5a antagonist component of the combination preferably ranges from about 0.001 mg per kg of body weight per day (mg/kg/day) to about 50.0 mg/kg/day, more preferably 0.005-20.0 mg/kg/day and most preferably 0.005-10.0 mg/kg/day. Suitable oral tablets and capsules for human patients contain between about 0.1 mg and 5.0 g, preferably between about 0.5 mg and 2.0 g, most preferably between about 0.5 mg and 1.0 g, for example, 0.5 mg, 1 mg, 5 mg, 10 mg, 50 mg, 150 mg, 250 mg, or 500 mg of C5a antagonist receptor antagonist. Oral administration may be in one or divided doses of two, three, or four times daily. Preferably fewer daily doses are preferred, with a single daily dose being most preferred. Preferably, for a human patient the oral dosage amount of the C5a antagonist component of the combination is from about 1 to 200 mg/day, and more preferably from about 5 to 160 mg/day. However, dosage amounts will vary depending on the potency, solubility and bioavailability of the specific C₅a antagonist used as well as other factors noted above.

Intravenously, the most preferred doses for the C5a antagonist component of the combination will range from about 0.5 pg to about 5 mg/kg/minute during a constant rate infusion, to achieve a plasma level concentration during the period of time of administration of between 0.1 ng/ml and 1.0 mg/ml.

The combination therapy methods of the invention include administration of a single pharmaceutical dosage formulation which contains both the C5a antagonist and the C5a receptor-inactive therapeutic agent, as well as administration of each active agent in its own separate pharmaceutical dosage formulation. The C5a receptor antagonist can be administered by a number of methods, for example orally, parenterally, transdermally, intravenously, intranasally, intra-articularly, or by any other known method for pharmaceutical administration. Oral, intranasal or topical dosing is generally preferred.

Within certain embodiments, all active agents of the instant combination therapy are administered orally, and the active agents are combined in a single oral dosage formulation. For example, a C5a antagonist and the C5a receptor-inactive therapeutic agent can be administered to the patient together in one oral composition such as a tablet or capsule. Alternatively, the combination therapy may comprise administration of an oral preparation of a C5a antagonist with a separate oral preparation of a C5a receptor-inactive therapeutic agent, or with a separate intravenous, transdermal, intraocular, intranasal, or intra-articular preparation of a C5a receptor-inactive therapeutic agent.

Where separate dosage formulations are used, the combination of C5a antagonist and the C5a receptor-inactive therapeutic agent can result from administration in various temporal relationships (e.g., at essentially the same time oor concurrently, or at separately staggered times, sequentially in either order). As used herein, combination administration and combination therapy include all such regimens that result in both the C5a antagonist and the C5a receptor-inactive therapeutic agent providing therapeutic effects that overlap in time with each other. With the exception of combination treatments where one or more of the therapeutic agents are administered by direct application to an affected area (e.g., by topical administration or injection), such effect is generally achieved when target blood level concentrations of each active agent occur at substantially the same time. As used herein “combination” indicates both composition of matter and method of treatment.

The dosage regimen utilizing a C5a antagonist and a C5a receptor-inactive therapeutic agent is selected in accordance with a variety of factors including species, age, weight, sex, medical condition of the patient, and other pharmaceutical agents that are being administered to the patient during treatment in accordance with the present invention. These factors further include the severity of the condition to be treated; the route of administration; the renal and hepatic function of the patient; and the particular compounds (including any salts or prodrugs thereof) employed. In some cases, the therapy may be administered on a long-term chronic basis, such as a period of several months or years, for as long as deemed medically appropriate for the patient.

Pharmaceutical Kits

The present invention also provides pharmaceutical kits comprising at least one C5a antagonist and at least one C5a receptor-inactive therapeutic agent together with indicia comprising instructions for using the contents of the kit to treat a patient suffering from a condition with a pathogenic inflammatory component. Such conditions include those described above, such as arthritis, asthma, psoriasis, reperfusion, traumatic brain and spinal cord injury, and cardio- and cerebrovascular disease. The active agents in a pharmaceutical kit (i.e., the C5a antagonist(s) and the C5a receptor-inactive therapeutic agent(s)), may be formulated for administration in a single pharmaceutical preparation, such as a formulation of both active agents in one tablet or capsule. Alternatively, one or more of the active agents of the pharmaceutical kit may be formulated for separate administration, for example by different routes of administration.

The packaging for the kit may be designed and manufactured in a variety of ways. For example, a pharmaceutical kit for oral administration of the active agents comprise a blister package containing rows of a C5a antagonist tablet and a tablet containing a C5a receptor-inactive therapeutic agent (e.g., COX-2 inhibitor or methotrexate), placed side by side on the same blister card, one each of the two types of tablets in its own blister bubble (or one each of the two types of tablets in a single blister bubble with no other tablets), with indicia on the card directing that one “pair” of tablets (i.e., one C5a antagonist tablet and one C5a receptor-inactive therapeutic agent) is to be ingested per day.

Kits for the Treatment of Arthritis

More particularly, the pharmaceutical kit is comprised of a C5a receptor-inactive therapeutic agent selected from non-steroidal anti-inflammatory drugs (NSAIDs) including cyclooxygenase enzyme inhibitors, such as salicylates, and particularly including cyclooxygenase-2 enzyme specific inhibitors, such as rofecoxib or celecoxib; corticosteroids, such as cortisone, prednisolone and prednisone; gold compounds including injectible gold sodium thiomalate and gold compounds formulated for oral administration; methotrexate; dihydroorotate dehydrogenase inhibitors, such as leflunomide; anti-C5 monoclonal antibodies; TNF alpha antagonists, such as entanercept or infliximab; and IL-1 receptor antagonists, such as anakinra.

One example of this embodiment is a pharmaceutical kit comprised of an oral dosage formulation of a C5a antagonist and an oral dosage formulation of aspirin and instructions for using the contents of the pharmaceutical kit to treat rheumatoid arthritis. Another example of this embodiment is a pharmaceutical kit comprised of a C5a antagonist and a COX-2 inhibitor formulated for either single or separate oral administration together with instructions for using the contents of the pharmaceutical kit to treat rheumatoid arthritis.

Kits for the Treatment of Asthma

In another embodiment, the pharmaceutical kit provided is comprised of a C5a receptor antagonist and a C5a receptor-inactive therapeutic agent for the treatment of asthma. The C5a receptor-inactive therapeutic agent for the treatment of asthma may be a corticosteroid, such as fluticasone propionate or triamcinolone. The C5a receptor-inactive therapeutic agent may also be cromolyn sodium, a long- or short-acting Beta adrenergic receptor agonist, particularly a Beta-2-receptor agonist, a leukotriene modifier, and anticholinergic, or a methylxanthine, for example, theophylline.

One example of this embodiment is a pharmaceutical kit comprised of an oral dosage formulation of a C5a receptor antagonist and an inhaled formulation of a Beta adrenergic receptor agonist, for example, albuterol. Other pharmaceutical kits of the invention for the treatment of asthma may also comprise inhaled formulations of both the C5a receptor antagonist and the C5a receptor-inactive therapeutic agent for the treatment of asthma, prepared as separate formulations or as a single formulation. When one agent of the kit is prepared as an inhaled formulation, an inhaler, nebulizer, or other device for inhalation may be provided in the kit.

Kits for the Treatment of Psoriasis

Pharmaceutical kits for the treatment of psoriasis comprised of a C5a-antagonist and a C5a receptor-inactive therapeutic agent for the treatment of psoriasis which is preferably selected from therapeutic agents the inhibit the hyperfunctional proliferation of epidermal cells; inhibit the inflammatory response; promote immunomodulation; and/or inhibit infection by bacteria and fungi. Included in the invention are combinations in which the C5a receptor-inactive therapeutic agent of the kit is a non-steroidal anti-inflammatory drug (NSAID), a steroid, coal tar, a moisturizer, calendula plant extract, an agents that arrests the proliferation of cells during the metaphase stage of cell division; e.g., SLO-PHYLLIN™ or THEO-DUR SPRINKLE™), an angiogenesis inhibitor such as ganglioside GM3 or a GM3 analog, a phospholipase A2 inhibitor or an imidazole antifungal. A C5a receptor-inactive therapeutic agent included in a kit for the treatment of psoriasis may also be an immunomodulator, such as a folic acid antagonist, an inhibitor of interleukin-2 production, an immunosuppressant, a TNF-alpha modulators, and antileukotriene agents, or a Vitamin A derivatives.

The invention particularly includes kits for the treatment of psoriasis in which one or both or the C5a antagonist and C5a receptor-inactive therapeutic agent is formulated for topical administration.

Kits for the Treatment of Cardio- and Cerebrovascular Disease

Pharmaceutical kits for the treatment of cardio- and cerbrovascular disease comprised of a C5a receptor-inactive therapeutic agent selected from the group that includes non-steroidal anti-inflammatory drugs (NSAIDs), cholesterol lowering drugs, particularly 3-hydroxy-3-methylglutaryl coenzyme A (HMG-CoA) reductase inhibitors (HMG-COA RI) such as lovastatin, pravastatin, or simvastatin, anti-hypertensive agents, including adrenergic receptor stimulators, including agonists, alpha/beta adrenergic receptor antagonists, beta adrenergic receptor antagonists, angiotensin converting enzyme (ACE) inhibitors, angiotensin II receptor antagonists, calcium channel blockers, diuretics and periphereal vasodilators, and platelet aggregation inhibitors, particularly GP IIb/IIa inhibitors, PDE inhibitors, particularly PDE III inhibitors, inhibitors of megakaryocyte hypermaturation, and ADP receptor inhibitors such as clopidogrel.

An example of this embodiment is a pharmaceutical kit comprising an oral dosage formulation of a C5a antagonist and an oral dosage formulation of simvastatin (ZOCOR™) and instructions for using the contents of the pharmaceutical kit to reduce the risk of myocardial infarction and/or stroke.

Pharmaceutical Preparations

The compounds for use in the described combination therapy may be administered orally, topically, transdermally, parenterally, by inhalation or spray in dosage unit formulations containing conventional non-toxic pharmaceutically acceptable carriers, adjuvants and vehicles. The term parenteral as used herein includes subcutaneous, intravenous, intramuscular, intrathecal and like types of injection or infusion techniques. One or more compounds of the combination may be present in association with one or more non-toxic pharmaceutically acceptable carriers and/or diluents and/or adjuvants and if desired other active ingredients. The pharmaceutical compositions containing compounds of the combination may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Compounds for use in the described combination therapy may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. Compounds used in the described combination therapy may be prepared as a sterile injectible aqueous or oleaginous suspension. This suspension may be formulated according to the known art using those suitable dispersing or wetting agents and suspending agents that have been mentioned above. The sterile injectible preparation may also be sterile injectible solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectibles.

Compounds of for use in the combination therapy may be administered parenterally in a sterile medium. The drug, depending on the vehicle and concentration used, can either be suspended or dissolved in the vehicle. Advantageously, one or more adjuvants such as preservatives, buffering agents, or local anesthetics can also be present in the vehicle.

For inhalation formulations, the compounds of the present invention may be delivered via any inhalation methods known to those skilled in the art. Such inhalation methods and devices include, but are not limited to, metered dose inhalers with propellants such as CFC or HFA or propellants that are physiologically and environmentally acceptable. Other included devices are breath operated inhalers, multidose dry powder inhalers and aerosol nebulizers.

Formulation suitable for administration by inhalation includes formulations of the active ingredient in a form that can be dispensed by such inhalation devices known to those in the art. Such formulations may include carriers such as powders and aerosols. The inhalant compositions used in the present invention may comprise liquid or powdered compositions containing the active ingredient that are suitable for nebulization and intrabronchial use, or aerosol compositions administered via an aerosol unit dispensing metered doses.

Suitable liquid compositions comprise the active ingredient in an aqueous, pharmaceutically acceptable inhalant solvent, e.g., isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs.

Suitable powder compositions include, by way of illustration, powdered preparations of the active ingredient thoroughly intermixed with lactose or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation.

Aerosol formulations for use in the subject method would typically include propellants, surfactants and co-solvents and may be filled into conventional aerosol containers that are closed by a suitable metering valve.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Within certain embodiments, compositions provided herein are formulated for topical administration. Such formulations typically comprise a topical vehicle combined with active agent(s), with or without additional optional components. Topical formulations are useful, for example, in the treatment of psoriasis and sunburn.

Suitable topical vehicles and additional components are well known in the art, and it will be apparent that the choice of a vehicle will depend on the particular physical form and mode of delivery. Topical vehicles include water; organic solvents such as alcohols (e.g., ethanol or isopropyl alcohol) or glycerin; glycols (e.g., butylene, isoprene or propylene glycol); aliphatic alcohols (e.g., lanolin); mixtures of water and organic solvents and mixtures of organic solvents such as alcohol and glycerin; lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile); and hydrocarbon-based materials such as microsponges and polymer matrices. A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatin-microcapsules, liposomes, albumin microspheres, microemulsions, nanoparticles or nanocapsules.

The topical formulations provided herein may be prepared in a variety of physical forms. For example, solids, pastes, creams, foams, lotions, gels, powders, aqueous liquids and emulsions are all contemplated by the present invention. The physical appearance and viscosity of such forms can be governed by the presence and amount of emulsifiers and viscosity adjusters present in the formulation. Particular topical formulations can often be prepared in a variety of these forms. Solids are generally firm and non-pourable and commonly are formulated as bars or sticks, or in particulate form; solids can be opaque or transparent, and optionally can contain solvents, emulsifiers, moisturizers, emollients, fragrances, dyes/colorants, preservatives and other active ingredients that increase or enhance the efficacy of the final product. Creams and lotions are often similar to one another, differing mainly in their viscosity; both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams may also contain solvents, emulsifiers, moisturizers, emollients, fragrances, dyes/colorants, preservatives and other active ingredients that increase or enhance the efficacy of the final product. Liquids are thinner than creams, lotions, or gels and often do not contain emulsifiers. Liquid topical products often contain solvents, emulsifiers, moisturizers, emollients, fragrances, dyes/colorants, preservatives and other active ingredients that increase or enhance the efficacy of the final product.

Suitable emulsifiers for use in topical formulations include, but are not limited to, ionic emulsifiers, behentirmonium methosulfate, cetearyl alcohol, non-ionic emulsifiers like polyoxyethylene oleyl ether, PEG-40 sterate, ceteareth-12, ceteareth-20, ceteareth-30, ceteareth alcohol, PEG-100 stearate and glyceryl stearate. Suitable viscosity adjusting agents include, but are not limited to, protective colloids or non-ionic gums such as hydroxyethylcellulose, xanthan gum, magnesium aluminum silicate, silica, microcrystalline wax, beeswax, paraffin, and cetyl palmitate. A gel composition may be formed by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations. Suitable preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerin, propylene glycol, and butylene glycol. Suitable emollients include lanolin alcohol, lanolin, lanolin derivatives, cholesterol, petrolatum, isostearyl neopentanoate and mineral oils. Suitable fragrances and colors for use in the formulations of the present invention include, but are not limited to, FD&C Red No. 40 and FD&C Yellow No. 5. Other suitable additional ingredients that may be included a topical formulation include, but are not limited to, abrasives, absorbents, anti-caking agents, anti-foaming agents, anti-static agents, astringents (e.g., witch hazel, alcohol and herbal extracts such as chamomile extract), binders/excipients, buffering agents, chelating agents, film forming agents, conditioning agents, propellants, opacifying agents, pH adjusters and protectants.

An example of a suitable topical vehicle for formulation of a gel is: hydroxypropylcellulose (2.1%); 70/30 isopropyl alcohol/water (90.9%); propylene glycol (5.1%); and Polysorbate 80 (1.9%). An example of a suitable topical vehicle for formulation as a foam is: cetyl alcohol (1.1%); stearyl alcohol (0.5%; Quaternium 52 (1.0%); propylene glycol (2.0%); Ethanol 95 PGF3 (61.05%); deionized water (30.05%); P75 hydrocarbon propellant (4.30%). All percents are by weight.

Typical modes of delivery for topical compositions include application using the fingers; application using a physical applicator such as a cloth, tissue, swab, stick or brush; spraying (including mist, aerosol or foam spraying); dropper application; sprinkling; soaking; and rinsing. Controlled release vehicles can also be used to administer the compounds of the present invention. The technology and products in this art are variably referred to as controlled release, sustained release, prolonged action, depot, repository, delayed action, retarded release and timed release; the words “controlled release” as used herein is intended to incorporate each of the foregoing technologies.

Numerous controlled release vehicles are known, including biodegradable or bioerodable polymers such as polylactic acid, polyglycolic acid, and regenerated collagen. Controlled release drug delivery devices include creams, lotions, tablets, capsules, gels, microspheres and liposomes. Transdermal formulations, from which active ingredients are slowly released are also well known and can be used in the present invention. Controlled release preparations can be achieved by the use of polymers to complex or absorb the active agent(s). The controlled delivery can be exercised by selecting appropriate macromolecule such as polyesters, polyamino acids, polyvinylpyrrolidone, ethylenevinyl acetate, methylcellulose, carboxymethylcellulose, and protamine sulfate, and the concentration of these macromolecule as well as the methods of incorporation are selected in order to control release of active compound.

Hydrogels, wherein the active agent(s) are dissolved in an aqueous constituent to gradually release over time, can be prepared by copolymerization of hydrophilic mono-olefinic monomers such as ethylene glycol methacrylate. Matrix devices, wherein the active agent(s) are dispersed in a matrix of carrier material, can be used. The carrier can be porous, non-porous, solid, semi-solid, permeable or impermeable. Alternatively, a device comprising a central reservoir of active agent(s) surrounded by a rate controlling membrane can be used to control the release of active agent(s). Rate controlling membranes include, for example, ethylene-vinyl acetate copolymer and butylene terephthalate/polytetramethylene ether terephthalate.

The following Examples are offered by way of illustration and not by way of limitation. Unless otherwise specified all reagents and solvent are of standard commercial grade and are used without further purification.

### EXAMPLES

### Example 1

### Expression of a C5A Receptor

A human C5a receptor cDNA is obtained by PCR using 1) a forward primer adding a Kozak ribosome binding site and 2) a reverse primer that added no additional sequence, and 3) an aliquot of a Stratagene Human Fetal Brain cDNA library as template. The sequence of the resulting PCR product is as described by Gerard and Gerard, (1991) Nature 349:614-17. The PCR product is subcloned into the cloning vector pCR-Script AMP (STRATAGENE, La Jolla, Calif.) at the Srf I site. It is then excised using the restriction enzymes EcoRI and NotI and subcloned in the appropriate orientation for expression into the baculoviral expression vector pBacPAK 9 (CLONTECH, Palo Alto, Calif.) that has been digested with EcoRI and NotI.

### Example 2

### Baculoviral Preparations for C5A Expression

The human C5a (hC5a) receptor baculoviral expression vector is co-transfected along with BACULOGOLD DNA (BD PharMingen, San Diego, Calif.) into Sf9 cells. The Sf9 cell culture supernatant is harvested three days post-transfection. The recombinant virus-containing supernatant is serially diluted in Hink's TNM-FH insect medium (JRH Biosciences, Lenexa, Kans.) supplemented Grace's salts and with 4. 1 mM L-Gln, 3.3 g/L LAH, 3.3 g/L ultrafiltered yeastolate and 10% heat-inactivated fetal bovine serum (hereinafter “insect medium”) and plaque assayed for recombinant plaques. After four days, recombinant plaques are selected and harvested into 1 ml of insect medium for amplification. Each 1 ml volume of recombinant baculovirus (at passage 0) is used to infect a separate T25 flask containing 2×10⁶ Sf9 cells in 5 mls of insect medium. After five days of incubation at 27° C., supernatant medium is harvested from each of the T25 infections for use as passage I inoculum.

Two of seven recombinant baculoviral clones are then chosen for a second round of amplification, using 1 ml of passage 1 stock to infect 1×10⁸ cells in 100 ml of insect medium divided into 2 T175 flasks. Forty-eight hours post infection, passage 2 medium from each 100 ml prep is harvested and plaque assayed for titer. The cell pellets from the second round of amplification are assayed by affinity binding as described below to verify recombinant receptor expression. A third round of amplification is then initiated using a multiplicity of infection of 0.1 to infect a liter of Sf9 cells. Forty hours post-infection the supernatant medium is harvested to yield passage 3 baculoviral stock.

The remaining cell pellet is assayed for affinity binding using the “Binding Assays” essentially as described by DeMartino et al. (1994) J. Biol. Chem. 269:14446-50 at page 14447, adapted as follows. Radioligand is 0.005-0.500 nM [¹²⁵I]C5a (human recombinant; New England Nuclear Corp., Boston, Mass.); the hC5a receptor-expressing baculoviral cells are used instead of 293 cells; the assay buffer contains 50 mM Hepes pH. 7.6, 1 mM CaCl₂, 5 mM MgCl₂, 0.1% BSA, pH 7.4, 0.1 mM bacitracin, and 100 KIU/ml aprotinin; filtration is carried out using GF/C WHATMAN filters (presoaked in 1.0% polyethyeneimine for 2 hours prior to use); and the filters are washed twice with 5 mLs cold binding buffer without BSA, bacitracin, or aprotinin.

Titer of the passage 3 baculoviral stock is determined by plaque assay and a multiplicity of infection, incubation time course, binding assay experiment is carried out to determine conditions for optimal receptor expression. A multiplicity of infection of 0.1 and a 72-hour incubation were the best infection parameters found for hC5a receptor expression in up to 1-liter Sf9 cell infection cultures.

### Example 3

### Baculoviral Infections

Log-phase Sf9 cells (INVITROGEN Corp., Carlsbad Calif.) are infected with one or more stocks of recombinant baculovirus followed by culturing in insect medium at 27° C. Infections are carried out either only with virus directing the expression of the hC5a receptor or with this virus in combination with three G-protein subunit-expression virus stocks: 1) rat G□ᵢ₂ G-protein-encoding virus stock (BIOSIGNAL #V5J008), 2) bovine b1 G-protein-encoding virus stock (BIOSIGNAL #V5H012), and 3) human g2 G-protein-encoding virus stock (BIOSIGNAL #V6B003), all of which may be obtained from BIOSIGNAL Inc. (Montreal, Canada).

The infections are conveniently carried out at a multiplicity of infection of 0.1:1.0:0.5:0.5. At 72 hours post-infection, a sample of cell suspension is analyzed for viability by trypan blue dye exclusion, and the remaining Sf9 cells are harvested via centrifugation (3000 rpm/10 minutes/4° C.).

### Example 4

### Purified Recombinant Insect Cell Membranes

Sf9 cell pellets are resuspended in homogenization buffer (10 mM HEPES, 250 mM sucrose, 0.5 ug/ml leupeptin, 2 ug/ml Aprotinin, 200 uM PMSF, and 2.5 mM EDTA, pH 7.4) and homogenized using a POLYTRON homogenizer (setting 5 for 30 seconds). The homogenate is centrifuged (536×g/10 minutes/4° C.) to pellet the nuclei. The supernatant containing isolated membranes is decanted to a clean centrifuge tube, centrifuged (48,000×g/30 minutes, 4° C.) and the resulting pellet resuspended in 30 ml homogenization buffer. This centrifugation and resuspension step is repeated twice. The final pellet is resuspended in ice cold Dulbecco's PBS containing 5 mM EDTA and stored in frozen aliquots at −80° C. until needed. The protein concentration of the resulting membrane preparation (hereinafter “P2 membranes”) is conveniently measured using a Bradford protein assay (Bio-Rad Laboratories, Hercules, Calif.). By this measure, a 1-liter culture of cells typically yields 100-150 mg of total membrane protein.

### Example 5

### Radioligand Binding Assays

Purified P2 membranes, prepared by the method given above, are resuspended by Dounce homogenization (tight pestle) in binding buffer (50 mM Hepes pH. 7.6, 120 mM NaCl, 1 mM CaCl₂, 5 mM MgCl₂, 0.1% BSA, pH 7.4, 0.1 mM bacitracin, 100 KIU/ml aprotinin).

For saturation binding analysis, membranes (5-50 μg) are added to polypropylene tubes containing 0.005-0.500 nM [¹²⁵I]C5a (human (recombinant), New England Nuclear Corp., Boston, Mass.). Nonspecific binding is determined in the presence of 300 nM hC5a (Sigma Chemical Co., St. Louis, Mo.) and accounts for less than 10% of total binding. For evaluation of guanine nucleotide effects on receptor affinity, GTPyS is added to duplicate tubes at the final concentration of 50 μM.

For competition analysis, membranes (5-50 μg) are added to polypropylene tubes containing 0.030 nM [¹²⁵I]C5a (human). Non-radiolabeled displacers are added to separate assays at concentrations ranging from 10⁻¹⁰ M to 10⁻⁵ M to yield a final volume of 0.250 mL. Nonspecific binding is determined in the presence of 300 nM hC5a (Sigma Chemical Co., St. Louis, Mo.) and accounts for less than 10% of total binding. Following a 2-hour incubation at room temperature, the reaction is terminated by rapid vacuum filtration. Samples are filtered over presoaked (in 1.0% polyethyleneimine for 2 hours prior to use) GF/C WHATMAN filters and rinsed 2 times with 5 mLs cold binding buffer without BSA, bacitracin, or aprotinin. Remaining bound radioactivity is quantified by gamma counting. K₁ and Hill coefficient (“nH”) are determined by fitting the Hill equation to the measured values with the aid of SIGMAPLOT software (SPSS Inc., Chicago, Ill.).

### Example 6

### Agonist-Induced GTP Binding

Agonist-stimulated GTP-gamma ³⁵S binding (“GTP binding”) activity can be used to identify agonist and antagonist compounds and to differentiate neutral antagonist compounds from those that possess inverse agonist activity. This activity can also be used to detect partial agonism mediated by antagonist compounds. A compound being analyzed in this assay is referred to herein as a “test compound.” Agonist-stimulated GTP binding activity is measured as follows: Four independent baculoviral stocks (one directing the expression of the hC5a receptor and three directing the expression of each of the three subunits of a heterotrimeric G-protein) are used to infect a culture of Sf9 cells as described in Example 3.

Agonist-stimulated GTP binding on purified membranes (prepared as described in Example 9) is assessed using hC5a (Sigma Chemical Co., St. Louis, Mo.) as agonist in order to ascertain that the receptor/G-protein-alpha-beta-gamma combination(s) yield a functional response as measured by GTP binding.

P2 membranes are resuspended by Dounce homogenization (tight pestle) in GTP binding assay buffer (50 mM Tris pH 7.0, 120 mM NaCl, 2 mM MgCl2, 2 mM EGTA, 0.1% BSA, 0.1 mM bacitracin, 100KIU/mL aprotinin, 5 μM GDP) and added to reaction tubes at a concentration of 30 μg protein/reaction tube. After adding increasing doses of the agonist hC5a at concentrations ranging from 10⁻¹² M to 10⁻⁶ M, reactions are initiated by the addition of 100 pM GTP-gamma ³⁵S. In competition experiments, non-radiolabeled test compounds are added to separate assays at concentrations ranging from 10⁻¹⁰ M to 10⁻⁵ M along with 10 nM hC5a to yield a final volume of 0.25 mL.

Neutral antagonists are those test compounds that reduce the C5a-stimulated GTP binding activity towards, but not below, baseline (the level of GTP bound by membranes in this assay in the absence of added C5a or other agonist and in the further absence of any test compound).

In contrast, in the absence of added C5a certain preferred compounds will reduce the GTP binding activity of the receptor-containing membranes below baseline, and are thus characterized as inverse agonists. If a test compound that displays antagonist activity does not reduce the GTP binding activity below baseline in the absence of the C5a agonist, it is characterized as a neutral antagonist.

An antagonist test compound that elevates GTP binding activity above baseline in the absence of added hC5a in this GTP binding assay is characterized as having partial agonist activity. Preferred antagonist compounds do not elevate GTP binding activity under such conditions more than 10%, 5% or 2% above baseline.

Following a 60-minute incubation at room temperature, the reactions are terminated by vacuum filtration over GF/C filters (pre-soaked in wash buffer, 0.1% BSA) followed by washing with ice-cold wash buffer (50 mM Tris pH 7.0, 120 mM NaCl). The amount of receptor-bound (and thereby membrane-bound) GTP-gamma ³⁵S is determined by measuring the bound radioactivity, preferably by liquid scintillation spectrometry of the washed filters. Non-specific binding is determined using 10 mM GTP-gamma ³⁵S and typically represents less than 5 percent of total binding. Data is expressed as percent above basal (baseline). The results of these GTP binding experiments may be conveniently analyzed using SIGMAPLOT software.

### Example 7

### Calcium Mobilization Assays

A. Response to C5a

U937 cells are grown in differentiation media (1 mM dibutyrl cAMP in RPMI 1640 medium containing 10% fetal bovine serum) for 48 hrs at 37° C. then reseeded onto 96-well plates suitable for use in a FLIPR™ Plate Reader (Molecular Devices Corp., Sunnyvale Calif.). Cells are grown an additional 24 hours (to 70-90% confluence) before the assay. The cells are then washed once with Krebs Ringer solution. FLUO-3 calcium sensitive dye (Molecular Probes, Inc. Eugene, Oreg.) is added to 10 μg/mL and incubated with the cells at room temperature for 1 to 2 hours. The 96 well plates are then washed to remove excess dye. Fluorescence responses, measured by excitation at 480 nM and emission at 530 nM, are monitored upon the addition of human C5a to the cells to a final concentration of 0.01-30.0 nM, using the FLIPR™ device (Molecular Devices). Differentiated U937 cells typically exhibit signals of 5,000-50,000 Arbitrary Fluorescent Light Units in response to agonist stimulation.

B. Assays for Determination of ATP Responses

Differentiated U937 cells (prepared and tested as described above under “A. Response to C5a”) are stimulated by the addition of ATP (rather than C5a) to a final concentration of 0.01 to 30 μM. This stimulation typically triggers a signal of 1,000 to 12,000 arbitrary fluorescence light units. Certain preferred compounds produce less than a 10%, less than a 5%, or less than a 2% alteration of this calcium mobilization signal when this control assay is carried out in the presence of the compound, as compared to the signal when the assay is performed in the absence of the compound.

C. Assays for the Identification of Receptor Modulatory Agents: Antagonists and Agonists

The calcium mobilization assay described above may be readily adapted for identifying test compounds that have agonist or antagonist activity at the human C5a receptor.

For example, in order to identify antagonist compounds, differentiated U937 cells are washed and incubated with Fluo-3 dye as described above. One hour prior to measuring the fluorescence signal, a subset of the cells is incubated with 1 μM of at least one compound to be tested. The fluorescence response upon the subsequent addition of 0.3 nM (final concentration) human recombinant C5a is monitored using the FLIPR™ plate reader. Antagonist compounds elicit at least a 2-fold decrease in the fluorescence response relative to that measured in the presence of human C5a alone. Preferred antagonist compounds elicit at least a 5-fold, preferably at least a 10-fold, and more preferably at least a 20-fold decrease in the fluorescence response relative to that measured in the presence of human C5a alone. Agonist compounds elicit an increase in fluorescence without the addition of C5a, which increase will be at least partially blocked by a known C5a receptor antagonist.

### Example 8

### Assay for C5A Receptor Mediated Chemotaxis

This assay is a standard assay of C5a receptor mediated chemotaxis.

Human promonocytic U937 cells or purified human or non-human neutrophilis are treated with dibutyryl cAMP for 48 hours prior to performing the assay. Human neutrophils or those from another mammalian species are used directly after isolation. The cells are pelleted and resuspended in culture media containing 0.1% fetal bovine serum (FBS) and 10 ug/ml calcein AM (a fluorescent dye). This suspension is then incubated at 37° C. for 30 minutes such that the cells take up the fluorescent dye. The suspension is then centrifuged briefly to pellet the cells, which are then resuspended in culture media containing 0.1% FBS at a concentration of approximately 3×10⁶ cells/mL. Aliquots of this cell suspension are transferred to clean test tubes, which contain vehicle (1% DMSO) or varying concentrations of a compound of interest, and incubated at room temperature for at least 30 minutes. The chemotaxis assay is performed in CHEMO TX 101-8, 96 well plates (Neuro Probe, Inc. Gaithersburg, Md.). The bottom wells of the plate are filled with medium containing 0-10 nM of C5a, preferably derived from the same species of mammal as are the neutrophils or other cells (e.g., human C5a for the human U937 cells). The top wells of the plate are filled with cell suspensions (compound or vehicle-treated). The plate is then placed in a tissue culture incubator for 60 minutes. The top surface of the plate is washed with PBS to remove excess cell suspension. The number of cells that have migrated into the bottom well is then determined using a fluorescence reader. Chemotaxis index (the ratio of migrated cells to total number of cells loaded) is then calculated for each compound concentration to determine an IC₅₀ value.

As a control to ensure that cells retain chemotactic ability in the presence of the compound of interest, the bottom wells of the plate may be filled with varying concentrations chemo-attractants that do not mediate chemotaxis via the C5a receptor (e.g., zymosan-activated serum (ZAS), N-formylmethionyl-leucyl-phenylalanine (FMLP) or leukotriene B4 (LTB4)), rather than C5a, under which conditions the compounds provided herein preferably do not inhibit chemotaxis.

Preferred compounds exhibit IC₅₀ values of less than 1 μM in the above assay for C5a receptor mediated chemotaxis.

### Example 9

### Pharmaceutical Preparations of Oral and Intravenous Administration

A. Tablets containing a C5a antagonist and an anti-arthritic agent which is not a C5a antagonist can be prepared as illustrated below:

| Ingredient | Amount |
|---|---|
| C5a antagonist | 5 mg-500 mg |
| C5a receptor-inactive therapeutic agent | 1 mg-500 mg |
| diluent, binder, distigrant, lubricant, excipients | q.s. 200-400 mg. |

B. Tablets containing a C5a antagonist as the only active ingredient can be prepared as illustrated below:

| Ingredient | mg | mg |
|---|---|---|
| C5a antagonist | 10 | 50 |
| Microcrystalline Cellulose | 70.4 | 352 |
| Granular Mannitol | 15.1 | 75.5 |
| Croscarmellose Sodium | 3.0 | 15.0 |
| Colloidal Silicon Dioxide | 0.5 | 2.5 |
| Magnesium Stearate (Impalpable Powder) | 1.0 | 5.0 |
| Total (mg) | 100 | 500 |

C. Tablets containing a C5a receptor antagonist and a C5a receptor inactive agent may be prepared as follows:

| Ingredient | mg | mg |
|---|---|---|
| C5a antagonist | 10 | 25 |
| C5a receptor inactive therapeutic agent | 10 | 25 |
| Microcrystalline Cellulose | 40 | 100 |
| Modified food corn starch | 1.05 | 4.25 |
| Magnesium sterate | 1.25 | 0.5 |

D. Intravenous formulations containing a C5a receptor antagonist and a C5a receptor inactive agent may be prepared as follows:

| Ingredient | Amount |
|---|---|
| C5a antagonist | 0.5-10 mg |
| C5a receptor inactive therapeutic agent | 0.5-10 mg |
| Sodium Citrate | 5-50 mg |
| Citric Acid | 1-15 mg |
| Sodium Chloride | 1-8 mg |
| Water for Injection | to 1.0 liter |

E. Oral suspensions containing a C5a receptor antagonist and a C5a receptor inactive agent may be prepared as follows:

| Ingredient | Amount per 5 ml dose |
|---|---|
| C5a antagonist | 5-100 mg |
| C5a receptor inactive therapeutic agent | 5-100 mg |
| Polyvinylpyrrolidone | 150 mg |
| Poly oxyethylene sorbitan monolaurate | 25 mg |
| | 10 mg to 5 mL |
| | with sorbitol solution |
| Benzoic Acid | (70%) |

### Example 10

### Representative C5A Antagonists

Table I provides representative C5a antagonists for use within the compositions and methods provided herein.

TABLE I

| C5a receptor antagonists useful in the combination therapies described | |
|---|---|
| herein. | |
| 1. | 1-(1-butyl)-2-phenyl-5-(N,N-di[3,4-methylenedioxyphenyl |
| | methyl])aminomethylimidazole |
| 2. | 1-(1-butyl)-2-phenyl-5-(1-[N-{3,4-methylenedioxyphenylmethyl}-N- |
| | phenylmethyl]amino)ethylimidazole |
| 3. | 1-Butyl-2-phenyl-4-bromo-5-(N-phenylmethyl-N-[1-butyl])amino-methylimidazole |
| 4. | 1-(1-Butyl)-2-phenyl-4-methyl-5-(N-[3,4-methylenedioxyphenyl-methyl]-N- |
| | phenylmethyl)aminomethylimidazole |
| 5. | 1-(1-Butyl)-2-(4-fluorophenyl)-5-(N-[1,4-benzodioxan-6-yl]methyl-N-phenylmethyl) |
| | aminomethylimidazole |
| 6. | 1-(1-Butyl)-2-(4-fluorophenyl)-5-(N-[3,4-methylenedioxyphenylmethyl]-N- |
| | phenylmethyl) aminomethylimidazole; |
| 7. | 1-(1-Butyl)-2-(2-fluorophenyl)-5-(N-[1,4-benzodioxan-6-ylmethyl]-N- |
| | phenylmethyl)amino-methylimidazole |
| 8. | 1-(1-Butyl)-2-(2-methoxyphenyl)-5-(N-[naphtha-2-ylmethyl]-N-phenylmethyl)amino- |
| | methylimidazole |
| 9. | 1-(1-Butyl)-2-(2-methoxyphenyl)-5-(N-[3,4-methylenedioxyphenylmethyl]-N- |
| | phenylmethyl) aminomethylimidazole |
| 10. | 1-(1-Butyl)-2-(2-methoxyphenyl)-5-(N,N-di[3,4-methylenedioxyphenylmethyl]) |
| | aminomethylimidazole |
| 11. | 1-(1-Butyl)-2-(2-methoxyphenyl)-5-(N-[4-dimethylaminophenylmethyl]-N- |
| | phenylmethyl) aminomethylimidazole |
| 12. | 1-(1-Butyl)-2-(2-methylphenyl)-5-(N-[3,4-methylenedioxyphenylmethyl]-N- |
| | phenylmethyl) aminomethylimidazole |
| 13. | 1-(1-Butyl)-2-(4-fluorophenyl)-5-(N,N-di[3,4-methylenedioxyphenylmethyl])amino- |
| | methylimidazole |
| 14. | 1-(1-Butyl)-2-(2-methylphenyl)-5-(N,N-di[3,4-methylenedioxyphenylmethyl])amino- |
| | methylimidazole |
| 15. | 1-(1-Butyl)-2-(3-fluorophenyl)-5-(N-[naphth-2-ylmethyl]-N-phenylmethyl)amino |
| | methylimidazole |
| 16. | 1-(1-Butyl)-2-(3-fluorophenyl)-5-(N-[3,4-methylenedioxyphenylmethyl]-N- |
| | phenylmethyl) aminomethylimidazole |
| 17. | 1-(1-Butyl)-2-(3-fluorophenyl)-5-(N,N-di[3,4-methylenedioxyphenylmethyl])amino- |
| | methylimidazole |
| 18. | 1-(1-Butyl)-2-(3-methoxyphenyl)-5-(N-[3,4-methylenedioxyphenymethyl]-N- |
| | phenylmethyl)-aminomethylimidazole |
| 19. | 1-(1-Butyl)-2-phenyl-5-{1-(N-[3,4-methylenedioxyphenylmethyl]-N- |
| | phenylmethyl)amino}ethylimidazole |
| 20. | 1-(1-Pentyl)-2-phenyl-5-(N-[indol-5-ylmethyl]-N-phenylmethyl) |
| | aminomethylimidazole |
| 21. | Bis-benzo[1,3]dioxol-5-ylmethyl-(3-butyl-2,5-diphenyl-3H-imidazol-4-ylmethyl)amine |
| 22. | Benzo[1,3]dioxol-5-ylmethyl-benzyl-[3-butyl-5-(4-methoxy-phenyl)-2-phenyl-3H- |
| | imdiazol-4-ylmethyl]-amine |
| 23. | 4-({Benzyl-[1-(3-butyl-2,5-diphenyl-3H-imidazol-4-yl)-ethyl)-amino}- |
| | methyl)benzamide |
| 24. | 4-{[Benzyl-(3-butyl-2,5-diphenyl-3H-imidazol-4-ylmethyl)-amino]-methyl}3-chloro- |
| | phenol |
| 25. | 4-({[1-(3-Butyl-2-phenyl-3H-imidazol-4-yl)-pentyl]-cyclohexymethyl-amino}-methyl)- |
| | phenol |
| 26. | 4-{[Benzyl-(3-butyl-2,5-diphenyl-3H-imidazol-4-ylmethyl)-amino]-methyl}benzamide |
| 27. | 1-(1-Propyl)-2-phenyl-5-(N-[indol-5-ylmethyl]-N-phenylmethyl) |
| | aminomethylimidazole |
| 28. | 1-(1-Butyl)-2-phenyl-5-(N-[1-(S)-phenylethyl]-N-phenylmethyl)aminomethylimidazole |
| 29. | 1-(1-Butyl)-2-phenyl-5-(N-[1-(R)-phenylethyl]-N-phenylmethyl)aminomethylimidazole |
| 30. | 1-(1-Butyl)-2-phenyl-5-(N-[3,4-methylenedioxyphenylmethyl]-N-[3,4- |
| | dichlorophenyl]methyl)aminomethylimidazole |
| 31. | 1-(1-Butyl)-2-phenyl-5-(N,N-di[3,4-methylenedioxyphenylmethyl]) |
| | aminomethylimidazole |
| 32. | 1-(1-Butyl)-2-phenyl-5-(N-[3,4-methylenedioxyphenylmethyl]-N-[3,4- |
| | methoxyphenylmethyl])-aminomethylimidazole |
| 33. | 1-(1-Butyl)-2-phenyl-5-(N-[3,4-methylenedioxyphenylmethyl]-N-[4-{1- |
| | propyl}phenylmethyl])aminomethylimidazole |
| 34. | 1-(1-Butyl)-2-phenyl-5-(N-[3,4-methylenedioxyphenylmethyl]-N-[3,4- |
| | dichlorophenylethyl])aminomethylimidazole |
| 35. | 1-(1-Butyl)-2-phenyl-5-(N-[3,4-methylenedioxyphenyl]methyl-N-[4- |
| | nitrophenylmethyl])aminomethylimidazole |
| 36. | 1-(1-Butyl)-2-phenyl-5-(N-[3,4-methylenedioxyphenylmethyl]-N-[4-{1-propyloxy} |
| | phenylmethyl])aminomethylimidazole |
| 37. | 1-(1-Butyl)-2-phenyl-5-(N-[3,4-methylenedioxyphenylmethyl]-N-[quinol-6-ylmethyl])- |
| | aminomethylimidazole |
| 38. | 1-(1-Butyl)-2-phenyl-5-(N-[3,4-methylenedioxyphenylmethyl]-N-[2,3- |
| | dichlorophenylmethyl])-aminomethylimidazole |
| 39. | 1-(1-Butyl)-2-phenyl-5-(N-[3,4-methylenedioxyphenylmethyl]-N-[3,4- |
| | dimethylphenylmethyl])-aminomethylimidazole |
| 40. | 1-(1-Butyl)-2-phenyl-5-(N-[3,4-methylenedioxyphenyl]methyl-N-[indan-2-yl])- |
| | aminomethylimidazole |
| 41. | 1-(1-Butyl)-2-phenyl-5-(N-[3,4-methylenedioxyphenylmethyl]-N-[2- |
| | phenylethyl])amino-methylimidazole |
| 42. | 1-(1-Propyl)-2-phenyl-5-(N-[1,4-benzodioxan-6-ylmethyl]-N- |
| | phenylmethyl)aminomethyl-imidazole |
| 43. | 1-(1-Butyl)-2-phenyl-5-(N-[3,4-methylenedioxyphenylmethyl]-N- |
| | phenylmethyl)aminomethyl-imidazole |
| 44. | 1-(1-Butyl)-2-phenyl-5-(N-[3,4-methylenedioxyphenylmethyl]-N- |
| | ethyl)aminomethylimidazole |
| 45. | 1-(1-Butyl)-2-phenyl-5-(N-[3,4-methylenedioxyphenylmethyl]-N-[1- |
| | propyl])aminomethyl-imidazole |
| 46. | 1-(1-Butyl)-2-phenyl-5-(N-[3,4-methylenedioxyphenylmethyl]-N-[1- |
| | butyl])aminomethyl-imidazole |
| 47. | 1-(1-Butyl)-2-phenyl-5-(N-[3,4-methylenedioxyphenylmethyl]-N- |
| | cycloheptylmethyl )amino-methylimidazole |
| 48. | 1-(1-Butyl)-2-phenyl-5-(N-[3,4-methylenedioxyphenylmethyl]-N- |
| | isobutyl)aminomethyl-imidazole |
| 49. | 1-(1-Butyl)-2-phenyl-5-(N-[3,4-methylenedioxyphenylmethyl]-N-[2- |
| | cyclopentylethyl])amino-methylimidazole |
| 50. | 1-(1-Butyl)-2-phenyl-5-(N-[3,4-methylenedioxyphenylmethyl]-N-[3- |
| | cyclopentylpropyl])amino-methylimidazole |
| 51. | 1-(1-Butyl)-2-phenyl-5-(N-[3,4-methylenedioxyphenylmethyl]-N-[1-n- |
| | octyl])aminomethyl-imidazole |
| 52. | 1-(1-Butyl)-2-phenyl-5-(N-[3,4-methylenedioxyphenylmethyl]-N- |
| | cyclopropylmethyl)amino-methylimidazole |
| 53. | 1-(1-Butyl)-2-phenyl-5-(N-[3,4-methylenedioxyphenylmethyl]-N- |
| | cyclopentylmethyl)amino-methylimidazole |
| 54. | 1-(1-Butyl)-2-phenyl-5-(N-[3,4-methylenedioxyphenylmethyl]-N- |
| | cyclohexylmethyl)amino-methylimidazole |
| 55. | 1-(1-Butyl)-2-phenyl-5-(N-[3,4-methylenedioxyphenylmethyl]-N-[t- |
| | amyl])aminomethylimidazole |
| 56. | 1-(1-Butyl)-2-phenyl-5-(N-[3,4-methylenedioxyphenylmethyl]-N-[1-{3- |
| | methyl}butyl)]amino-methylimidazole |
| 57. | 1-(1-Butyl)-2-phenyl-5-(N-[3,4-methylenedioxyphenylmethyl]-N-[1-{2,2- |
| | dimethyl}butyl]) aminomethylimidazole |
| 58. | 1-(1-Butyl)-2-phenyl-5-(N-[3,4-methylenedioxyphenylmethyl]-N- |
| | methyl)aminomethylimidazole |
| 59. | 1-(1-Butyl)-2-phenyl-5-(N-[3,4-methylenedioxyphenylmethyl]-N-[2- |
| | thiophenylmethyl])amino-methylimidazole |
| 60. | 1-(1-Butyl)-2-phenyl-5-(N-[3,4-methylenedioxyphenylmethyl]-N-[indol-5- |
| | ylmethyl])amino-methylimidazole |
| 61. | 1-(1-Butyl)-2-phenyl-5-(N-[3,4-methylenedioxyphenylmethyl]-N-[{1-methylindol-5- |
| | yl}methyl])aminomethylimidazole |
| 62. | 1-(1-Butyl)-2-phenyl-5-(N-[3,4-methylenedioxyphenyl]methyl-N-[4-hydroxy-2- |
| | chlorophenyl]-methyl)aminomethylimidazole |
| 63. | 1-(1-Butyl)-2-(3-fluorophenyl)-5-(1-[N-{2-chloro-4-hydroxyphenyl}methyl-N- |
| | phenylmethyl]) aminoethylimidazole |
| 64. | 1-(1-Butyl)-2-phenyl-5-(N-[3,4-methylenedioxyphenyl]methyl-N-[2,3- |
| | dihydrobenzo[b]furan-5-yl]methyl)aminomethylimidazole |
| 65. | 1-Butyl-2-(4-fluorophenyl)-5-(1-[N-{3,4-methylenedioxyphenyl}methyl-N- |
| | phenylmethyl]-amino)ethylimidazole |
| 66. | 1-(1-Butyl)-2-(2-thienyl)-5-(N-[3,4-methylenedioxyphenyl]methyl-N-phenylmethyl] |
| | aminomethylimidazole |
| 67. | 1-(1-Butyl)-2-phenyl-5-(N-[3,4,5-trimethoxyphenylmethyl]-N-phenylmethyl)amino- |
| | methylimidazole |
| 68. | 1-(1-Butyl)-2-phenyl-5-(N-phenylmethyl-N-[3,4- |
| | dimethoxyphenylmethyl])aminomethyl-imidazole |
| 69. | 1-(1-Butyl)-2-phenyl-5-(N-[4-dimethylaminophenylmethyl]-N- |
| | phenylmethyl)aminomethyl-imidazole |
| 70. | 1-(1-Butyl)-2-phenyl-5-(N-[4-methylaminophenylmethyl]-N- |
| | phenylmethyl)aminomethyl-imidazole |
| 71. | 1-(1-Butyl)-2-phenyl-5-(N-[3-methyl-4-aminophenylmethyl]-N- |
| | phenylmethyl)aminomethyl-imidazole) |
| 72. | 1-(1-Butyl)-2-phenyl-5-(N-[2,3-dichlorophenylmethyl]-N- |
| | phenylmethyl)aminomethylimidazole |
| 73. | 1-(1-Butyl)-2-phenyl-5-(N-[3,4-dichlorophenylmethyl]-N- |
| | phenylmethyl)aminomethylimidazole |
| 74. | 1-(1-Butyl)-2-phenyl-5-(N-[3,4-difluorophenylmethyl]-N- |
| | phenylmethyl)aminomethylimidazole |
| 75. | 1-(1-Butyl)-2-phenyl-5-(N-(benzo[b]thiophen-5-ylmethyl)-N- |
| | phenylmethyl)aminomethyl-imidazole |
| 76. | 1-(1-Butyl)-2-phenyl-5-(N-[4-ethoxyphenylmethyl]-N- |
| | phenylmethyl)aminomethylimidazole |
| 77. | 1-(1-Butyl)-2-phenyl-4-bromo-5-(N-phenylmethyl-N-[1-butyl])aminomethylimidazole |
| 78. | 1-(1-Butyl)-2-phenyl-5-(N-[4-methoxyphenylmethyl]-N- |
| | phenylmethyl)aminomethylimidazole |
| 79. | 1-(1-Butyl)-2-phenyl-5-(N-[6-chloro-3,4-methylenedioxyphenylmethyl]-N- |
| | phenylmethyl)-aminomethylimidazole |
| 80. | 1-(1-Butyl)-2-phenyl-5-(N-[2,3-dichlorophenylmethyl]-N-[1- |
| | butyl])aminomethylimidazole |
| 81. | 1-(1-Butyl)-2-phenyl-5-(N-[3-methoxyphenylmethyl]-N- |
| | phenylmethyl)aminomethylimidazole |
| 82. | 1-(1-Butyl)-2-phenyl-5-(N-[2-chloro-4-fluorophenylmethyl]-N- |
| | phenylmethyl)aminomethyl-imidazole |
| 83. | 1-(1-Butyl)-2-phenyl-4-bromo-5-(N-[2,3-dichlorophenylmethyl]-N-[1- |
| | butyl])aminomethyl-imidazole |
| 84. | 1-(1-Butyl)-2-phenyl-5-(N-[2,6-dichlorophenylmethyl]-N- |
| | phenylmethyl)aminomethylimidazole |
| 85. | 1-(1-Butyl)-2-phenyl-5-(N-[2-chloro-4-hydroxyphenylmethyl]-N- |
| | phenylmethyl)aminomethyl-imidazole |
| 86. | 1-(1-Butyl)-2-phenyl-4-chloro-5-(N-phenylmethyl-N-[1-butyl])aminomethylimidazole |
| 87. | 4-{[Benzyl-(3-butyl-2,5-diphenyl-3H-imidazol-4-ylmethyl)-amino]-methyl}-2-methyl- |
| | phenol |
| 88. | 4-{[(3-Butyl-2,5-diphenyl-3H-imidazol-4-ylmethyl]-cyclohexylmethyl-amino}- |
| | methyl)-2-methyl-phenol |
| 89. | (3-Butyl-2,5-diphenyl-3H-imidazol-4-ylmethyl)-(2,6-difluoro-benzyl)-(4-methoxy- |
| | benzyl)-amine |
| 90. | Benzo[1,3]dioxol-5-ylmethyl-butyl-[3-butyl-2-(2-methoxy-phenyl)-5-phenyl-3H- |
| | imidazol-4-ylmethyl]-amine |
| 91. | 4-({Benzyl-[3-butyl-2-(2-methoxy-phenyl)-5-phenyl-3H-imidazol-4-ylmethyl]-amino}- |
| | methyl)-benzenesulfonamide |
| 92. | Benzo[1,3]dioxol-5-ylmethyl-benzyl-[3-butyl-2-(2-methoxy-phenyl)-5-phenyl-3H- |
| | imidazol-4-ylmethyl]-amine |
| 93. | 4-({Butyl-[3-butyl-2-(3-methoxy-phenyl)-5-phenyl-3H-imidazol-4-ylmethyl]-amino}- |
| | methyl)-3-chloro-phenol |
| 94. | 4-{[(3-Butyl-2,5-diphenyl-3H-imidazol-4-ylmethyl)-(4-methoxy-benzyl)-amino]- |
| | methyl}-benzoic acid |
| 95. | 4-({Benzyl-[3-butyl-2-(3-methoxy-phenyl)-5-phenyl-3H-imidazol-4-ylmethyl]-amino}- |
| | methyl)-3-chloro-phenol |
| 96. | Benzo[1,3]dioxol-5-ylmethyl-benzyl-[1-(3-butyl-2,5-diphenyl-3H-imidazol-4-yl)- |
| | pentyl]-amine |
| 97. | Benzo[1,3]dioxol-5-ylmethyl-benzyl-[1-(3-butyl-2,5-diphenyl-3H-imidazol-4-yl)- |
| | ethyl]-amine |
| 98. | 4-{[Butyl-(3-butyl-2,5-diphenyl-3H-imidazol-4-ylmethyl)-amino]-methyl}benzamide |
| 99. | Benzo[1,3]dioxol-5-ylmethyl-benzyl-[3-butyl-5-(4-fluoro-phenyl)-2-phenyl-3H- |
| | imidazol-4-ylmethyl]-amine |
| 100. | 3-{[Benzyl-(3-butyl-2,5-diphenyl-3H-imidazol-4-ylmethyl)amino]-methyl}-phenol |
| 101. | 4-{[Butyl-(3-butyl-5-tert-butyl-2-phenyl-3H-imidazol-4-ylmethyl)-amino]- |
| | methyl}benzamide |
| 102. | Benzyl-(3-butyl-2,5-diphenyl-3H-imidazol-4-ylmethyl)-(2,3-dihydro-benzo[1,4]dioxin- |
| | 6-ylmethyl)-amine |
| 103. | (3-butyl-2,5-diphenyl-3H-imidazol-4-ylmethyl)-(2,5-difluoro-benzyl)-(4-methoxy- |
| | benzyl)-amine |
| 104. | (3-butyl-2,5-diphenyl-3H-imidazol-4-ylmethyl)-(2,6-dichloro-benzyl)-(4-methoxy- |
| | benzyl)-amine |
| 105. | 4-{[Benzyl-(3-butyl-2,5-diphenyl-3H-imidazol-4-ylmethyl)-amino]-methyl}-2,6- |
| | dimethyl-phenol |
| 106. | 4-({[3-Butyl-5-(4-methoxy-phenyl)-2-phenyl-3H-imidazol-4-ylmethyl]- |
| | cyclohexylmethyl-amino}-methyl)-2,6-dimethyl-phenol |
| 107. | [3-Butyl-5-(4-methoxy-phenyl)-2-phenyl-3H-imidazol-4-ylmethyl]-cyclohexylmethyl- |
| | (2,3,dihydro-benzofuran-5-ylmethyl)-amine |
| 108. | 4-{[Butyl-(3-butyl-2,5-diphenyl-3H-imidazol-4-ylmethyl)-amino]-methyl}-2,6- |
| | dimethyl-phenol |
| 109. | 4-({Butyl-[1-(3-butyl-2,5-diphenyl-3H-imidazol-4-yl)-ethyl)-amino}-methyl)-2,6- |
| | dimethyl-phenol |
| 110. | 4-{[(3-Butyl-2,5-diphenyl-3H-imidazol-4-ylmethyl)-(4-dimethylamino-benzyl)- |
| | methyl}-benzoic acid |
| 111. | 4-{5-[(Bis-benzo[1,3]dioxol-5-ylmethyl-amino)-methyl]-2,4-diphenyl-imidazol-1-yl}- |
| | butyric acid ethyl ester |
| 112. | 4-{5-[(Bis-benzo[1,3]dioxol-5-ylmethyl-amino)-methyl]-2,4-diphenyl-imidazol-1- |
| | yl}butan-1-ol |
| 113. | (4-{[(3-Butyl-2,5-diphenyl-3H-imidazol-4-ylmethyl)-cyclohexylmethyl- |
| | amino]methyl}-phenyl)-dimethyl-amine |
| 114. | 1-(1-Butyl)-2-phenyl-5-(N-[4-{1-pyrrolidinyl}phenylmethyl]-N- |
| | phenylmethyl)aminomethyl-imidazole; |
| 115. | 1-(1-Butyl)-2-phenyl-5-(N-[4-diethylaminophenylmethyl]-N- |
| | phenylmethyl)aminomethyl-imidazole; |
| 116. | 1-(1-Butyl)-2-phenyl-5-(N-[pyridin-2-ylmethyl]-N- |
| | phenylmethyl)aminomethylimidazole; |
| 117. | 1-(1-Butyl)-2-phenyl-5-(N-[pyridin-3-ylmethyl]-N- |
| | phenylmethyl)aminomethylimidazole; |
| 118. | 1-(1-Butyl)-2-phenyl-5-(N-[pyridin-4-ylmethyl]-N- |
| | phenylmethyl)aminomethylimidazole; |
| 119. | 1-(1-Butyl)-2-phenyl-5-(N-[2-fluoro-6-chlorophenylmethyl]-N- |
| | phenylmethyl)aminomethyl-imidazole); |
| 120. | 1-(1-Butyl)-2-phenyl-5-(N-[2,4-dichlorophenylmethyl]-N-phenylmethyl)aminomethyl- |
| | imidazole); |
| 121. | 1-(1-Butyl)-2-phenyl-5-(N-[4-chlorophenylmethyl]-N- |
| | phenylmethyl)aminomethylimidazole; |
| 122. | 1-(1-Butyl)-2-phenyl-5-(N-[4-hydroxyphenylmethyl]-N- |
| | phenylmethyl)aminomethylimidazole; |
| 123. | 1-(1-Butyl)-2-phenyl-5-(N-[4-trifluoromethoxyphenylmethyl]-N- |
| | phenylmethyl)aminomethyl-imidazole); |
| 124. | 1-(1-Butyl)-2-phenyl-5-(N-[2-chloro-3,4-dimethoxyphenylmethyl]-N- |
| | phenylmethyl)amino-methylimidazole); |
| 125. | 1-(1-Butyl)-2-phenyl-5-(N-[4-nitrophenylmethyl]-N- |
| | phenylmethyl)aminomethylimidazole; |
| 126. | 1-(1-Butyl)-2-phenyl-5-(N-[4-aminophenylmethyl]-N- |
| | phenylmethyl)aminomethylimidazole; |
| 127. | 1-(1-Butyl)-2,4-diphenyl-5-(N-phenylmethyl-N-[1-butyl])aminomethylimidazole; |
| 128. | 1-(1-Butyl)-2-phenyl-5-(N-[2-aminopyridin-5-ylmethyl]-N- |
| | phenylmethyl)aminomethyl-imidazole |
| 129. | 1-(1-Butyl)-2-phenyl-5-(N-[2,3-dihydrobenzo[b]furan-5-ylmethyl]-N- |
| | phenylmethyl)amino-methylimidazole; |
| 130. | 1-(1-Butyl)-2-phenyl-5-(N-[2-chloro-4-hydroxyphenylmethyl]-N-[1- |
| | butyl])aminomethyl-imidazole) |
| 131. | 1-(1-Butyl)-2-phenyl-4-methyl-5-(N-phenylmethyl-N-[1-butyl])aminomethylimidazole; |
| 132. | 1-(1-Butyl)-2-(4-fluorophenyl)-5-(N-[2-chloro-4-hydroxyphenylmethyl]-N- |
| | phenylmethyl)-aminomethylimidazole; |
| 133. | 1-(1-Butyl)-2-(3-fluorophenyl)-5-(N-[2-chloro-4-hydroxyphenylmethyl]-N- |
| | phenylmethyl)-aminomethylimidazole; |
| 134. | 1-(1-Butyl)-2-(3-fluorophenyl)-5-(N-[2,3-dichlorophenylmethyl]-N- |
| | phenylmethyl)amino-methylimidazole; |
| 135. | 1-(1-Butyl)-2-(3-fluorophenyl)-5-(N-[4-dimethylaminophenylmethyl]-N- |
| | phenylmethyl)amino-methylimidazole; |
| 136. | 1-(1-Butyl)-2-(3-fluorophenyl)-5-(N-[4-{1-pyrrolidinyl}phenylmethyl]-N- |
| | phenylmethyl)amino-methylimidazole; |
| 137. | 1-(1-Butyl)-2-(3-chlorophenyl)-5-(1-[N-{2-chloro-4-hydroxyphenylmethyl}-N- |
| | phenylmethyl]amino)ethylimidazole; |
| 138. | 1-(1-Butyl)-2-phenyl-5-(N-[indol-5-ylmethyl]-N-phenylmethyl)aminomethylimidazole |
| 139. | 1-(1-Butyl)-2-(4-fluorophenyl)-5-(1-N,N-di[3,4-methylenedioxyphenylmethyl] |
| | amino)ethylimidazole; |
| 140. | 2-{[5-({Butyl[(1-butyl-2,4-diphenylimidazol-5-yl)methyl]amino}methyl)-2- |
| | pyridyl]amino}ethan-1-ol; |
| 142. | 4-{[butyl(1-butyl-2-phenyl(4,5,6-trihydrocyclopenta[3,2-d]imidazol-6- |
| | yl))amino]methyl}-3-chlorophenol |
| 143. | 2-phenyl-4-(N,N-di{2H-Benzo[3,4-d]-1,3-dioxolan-5-ylmethyl}amino)methyl-3- |
| | butylpyridine |
| 144. | 1,3-diphenyl-4-(N-{2H-benzo[3,4-d]-1,3-dioxolan-5-ylmethyl}-N-butylamino)methyl- |
| | 5-propylpyrazole |
| 145. | N-(1-fluorobenzyl)-N-indan-2-yl-2-(6,7-dimethoxy-1-phenyl-1,2,3,4- |
| | tetrahydroisoquinolin-1-yl) acetamide |
| 146. | 4′Trifluoromethyl-biphenyl-2-carboxylic acid benzo[1,3]dioxol-5-ylmethyl-benzyl- |
| | amide |
| 147. | N-Benzo[1,3]dioxol-5-ylmethyl-N-benzyl-2-pyrazol-1-yl-benzamide |
| 148. | 1-[1-(2,3-dihydro-1H-indan-2-yl)-2-phenyl-1H-imidazol-5-yl]-N-(2-fluoro-5- |
| | methoxybenzyl)-N-(4-methoxybenzyl)methanamine |
| 149. | 1-(2-bromophenyl)-N-{[1-(2,3-dihydro-1H-indan-2-yl)-2-phenyl-1H-imidazol-5- |
| | yl]methyl}-N-(4-methoxybenzyl)methanamine |
| 150. | N-benzyl-1-(1-butyl-2-phenyl-1H-imidazol-5-yl)-N-(1H-indol-5- |
| | ylmethyl)methanamine |
| 151. | N-[(1-butyl-2-phenyl-1H-imidazol-5-yl)methyl]-N-(1H-indol-4-ylmethyl)-2- |
| | phenylethanamine |
| 152. | N-[(1-butyl-2-phenyl-1H-imidazol-5-yl)methyl]-N-(1H-indol-5-ylmethyl)-2- |
| | phenylethanamine |
| 153. | N-benzyl-1-(1-butyl-2-phenyl-1H-imidazol-5-yl)-N-(1-naphthylmethyl)methanamine |
| 154. | N-benzyl-1-(1-butyl-2-phenyl-1H-imidazol-5-yl)-N-(2-naphthylmethyl)methanamine |
| 155. | 1-(1,3-benzodioxol-5-yl)-N-benzyl-N-[(1-pentyl-2-phenyl-1H-imidazol-5- |
| | yl)methyl]methanamine |
| 156. | N-benzyl-1-(2,3-dihydro-1,4-benzodioxin-6-yl)-N-[(1-pentyl-2-phenyl-1H-imidazol-5- |
| | yl)methyl]methanamine |
| 157. | N-benzyl-1-(1-butyl-2-phenyl-1H-imidazol-5-yl)-N-(3,4- |
| | dimethoxybenzyl)methanamine |
| 158. | N-benzyl-N-[(1-butyl-2-phenyl-1H-imidazol-5-yl)methyl]butan-1-amine |
| 159. | N-benzyl-1-(1-butyl-2-phenyl-1H-imidazol-5-yl)-N-(2,3-dihydro-1,4-benzodioxin-6- |
| | ylmethyl)methanamine |
| 160. | N-benzyl-1-(2,3-dihydro-1,4-benzodioxin-6-yl)-N-[(2-phenyl-1-propyl-1H-imidazol-5- |
| | yl)methyl]methanamine |
| 161. | 1-(1,3-benzodioxol-5-yl)-N-benzyl-N-[(1-butyl-2-phenyl-1H-imidazol-5- |
| | yl)methyl]methanamine; |
| 162. | N-benzyl-N-[(1-butyl-2-phenyl-1H-imidazol-5-yl)methyl]-N-[4- |
| | (dimethylamino)benzyl]amine |
| 163. | N-benzyl-N-[(1-butyl-2-phenyl-1H-imidazol-5-yl)methyl]-N-[4- |
| | (methylamino)benzyl]amine; |
| 164. | N-(4-amino-3-methylbenzyl)-N-benzyl-N-[(1-butyl-2-phenyl-1H-imidazol-5- |
| | yl)methyl]amine; |
| 165. | N-benzyl-1-(1H-indol-5-yl)-N-[(2-phenyl-1-propyl-1H-imidazol-5- |
| | yl)methyl]methanamine; |
| 166. | 1-(1,3-benzodioxol-5-yl)-N-[(1-butyl-2-phenyl-1H-imidazol-5-yl)methyl]-N-(1H-indol- |
| | 5-ylmethyl)methanamine; |
| 167. | 1-(1,3-benzodioxol-5-yl)-N-(1,3-benzodioxol-5-ylmethyl)-N-[(1-butyl-2-phenyl-1H- |
| | imidazol-5-yl)methyl]methanamine; |
| 168. | N-(1,3-benzodioxol-5-ylmethyl)-N-[(1-butyl-2-phenyl-1H-imidazol-5-yl)methyl]-3- |
| | cyclopentylpropan-1-amine; |
| 169. | 1-(1,3-benzodioxol-5-yl)-N-[(1-butyl-2-phenyl-1H-imidazol-5-yl)methyl]-N- |
| | (cyclopentylmethyl)methanamine; |
| 170. | 1-(1,3-benzodioxol-5-yl)-N-benzyl-N-{[1-butyl-2-(2-fluorophenyl)-1H-imidazol-5- |
| | yl]methyl}methanamine; |
| 171. | N-benzyl-1-[1-butyl-2-(2-fluorophenyl)-1H-imidazol-5-yl]-N-(2,3-dihydro-1,4- |
| | benzodioxin-6-ylmethyl)methanamine; |
| 172. | N-benzyl-1-[1-butyl-2-(2-fluorophenyl)-1H-imidazol-5-yl]-N-(2- |
| | naphthylmethyl)methanamine; |
| 173. | N-benzyl-1-(1-butyl-2-phenyl-1H-imidazol-5-yl)-N-(2,3-dichlorobenzyl)methanamine; |
| 174. | 1-(1,3-benzodioxol-5-yl)-N-[(1-butyl-2-phenyl-1H-imidazol-5-yl)methyl]-N- |
| | (cyclohexylmethyl)methanamine; |
| 175. | 1-(1,3-benzodioxol-5-yl)-N-[(1-butyl-2-phenyl-1H-imidazol-5-yl)methyl]-N-(thien-2- |
| | ylmethyl)methanamine; |
| 176. | N-(1,3-benzodioxol-5-ylmethyl)-N-[(1-butyl-2-phenyl-1H-imidazol-5-yl)methyl]butan- |
| | 1-amine; |
| 177. | N-(1,3-benzodioxol-5-ylmethyl)-N-[(1-butyl-2-phenyl-1H-imidazol-5- |
| | yl)methyl]propan-1-amine; |
| 178. | 1-(1,3-benzodioxol-5-yl)-N-[(1-butyl-2-phenyl-1H-imidazol-5-yl)methyl]-N-(3,4- |
| | dichlorobenzyl)methanamine; |
| 179. | 1-(1,3-benzodioxol-5-yl)-N-[(1-butyl-2-phenyl-1H-imidazol-5-yl)methyl]-N-(3,4- |
| | dimethoxybenzyl)methanamine; |
| 180. | N-benzyl-1-(1H-indol-5-yl)-N-[(1-pentyl-2-phenyl-1H-imidazol-5- |
| | yl)methyl]methanamine; |
| 181. | N-benzyl-1-[1-(2-methoxyethyl)-2-phenyl-1H-imidazol-5-yl]-N-(1- |
| | naphthylmethyl)methanamine; |
| 182. | N-benzyl-1-[1-(2-methoxyethyl)-2-phenyl-1H-imidazol-5-yl]-N-(2- |
| | naphthylmethyl)methanamine; |
| 183. | methyl 4-({benzyl[(1-butyl-2-phenyl-1H-imidazol-5-yl)methyl]amino} |
| | methyl)phenyl(methyl)carbamate |
| 184. | methyl 4-({benzyl[(1-butyl-2-phenyl-1H-imidazol-5-yl)methyl]amino}methyl)-2- |
| | methylphenylcarbamate; |
| 185. | 1-(1-benzothien-5-yl)-N-benzyl-N-[(1-butyl-2-phenyl-1H-imidazol-5- |
| | yl)methyl]methanamine; |
| 186. | N-(1,3-benzodioxol-5-ylmethyl)-N-[(1-butyl-2-phenyl-1H-imidazol-5-yl)methyl]-2- |
| | (3,4-dichlorophenyl)ethanamine; |
| 187. | 1-(1,3-benzodioxol-5-yl)-N-[(1-butyl-2-phenyl-1H-imidazol-5-yl)methyl]-N-(4- |
| | propylbenzyl)methanamine; |
| 188. | N-(1,3-benzodioxol-5-ylmethyl)-N-[(1-butyl-2-phenyl-1H-imidazol-5-yl)methyl]-2- |
| | methylpropan-1-amine; |
| 189. | 1-(1,3-benzodioxol-5-yl)-N-[(1-butyl-2-phenyl-1H-imidazol-5-yl)methyl]-N-(4- |
| | propoxybenzyl)methanamine; |
| 190. | 1-(1,3-benzodioxol-5-yl)-N-benzyl-N-{[1-butyl-2-(2-methoxyphenyl)-1H-imidazol-5- |
| | yl]methyl}methanamine; |
| 191. | N-benzyl-1-[1-butyl-2-(2-methoxyphenyl)-1H-imidazol-5-yl]-N-(2- |
| | naphthylmethyl)methanamine; |
| 192. | 1-(1,3-benzodioxol-5-yl)-N-(1,3-benzodioxol-5-ylmethyl)-N-{[1-butyl-2-(2- |
| | methoxyphenyl)-1H-imidazol-5-yl]methyl}methanamine; |
| 193. | 4-[(benzyl{[1-butyl-2-(2-methoxyphenyl)-1H-imidazol-5-yl]methyl}amino)methyl]- |
| | N,N-dimethylaniline; |
| 194. | 1-(1,3-benzodioxol-5-yl)-N-benzyl-N-{[1-butyl-2-(4-methoxyphenyl)-1H-imidazol-5- |
| | yl]methyl}methanamine; |
| 195. | 4-[(benzyl{[1-butyl-2-(4-methoxyphenyl)-1H-imidazol-5-yl]methyl}amino)methyl]- |
| | N,N-dimethylaniline; |
| 196. | 1-(1,3-benzodioxol-5-yl)-N-(1,3-benzodioxol-5-ylmethyl)-N-{[1-butyl-2-(4- |
| | fluorophenyl)-1H-imidazol-5-yl]methyl}methanamine; |
| 197. | 1-(1,3-benzodioxol-5-yl)-N-benzyl-N-{[1-butyl-2-(2-methylphenyl)-1H-imidazol-5- |
| | yl]methyl}methanamine; |
| 198. | 1-(1,3-benzodioxol-5-yl)-N-(1,3-benzodioxol-5-ylmethyl)-N-{[1-butyl-2-(2- |
| | methylphenyl)-1H-imidazol-5-yl]methyl}methanamine; |
| 199. | 1-(1,3-benzodioxol-5-yl)-N-benzyl-N-{[1-butyl-2-(3-fluorophenyl)-1H-imidazol-5- |
| | yl]methyl}methanamine; |
| 200. | 1-(1,3-benzodioxol-5-yl)-N-(1,3-benzodioxol-5-ylmethyl)-N-{[1-butyl-2-(3- |
| | fluorophenyl)-1H-imidazol-5-yl]methyl}methanamine; |
| 201. | N-benzyl-1-[1-butyl-2-(3-fluorophenyl)-1H-imidazol-5-yl]-N-(2- |
| | naphthylmethyl)methanamine; |
| 202. | 1-(1,3-benzodioxol-5-yl)-N-benzyl-N-1{[1-butyl-2-(3-methoxyphenyl)-1H-imidazol-5- |
| | yl]methyl}methanamine; |
| 203. | 1-(1,3-benzodioxol-5-yl)-N-benzyl-N-{[1-butyl-2-(2-naphthyl)-1H-imidazol-5- |
| | yl]methyl}methanamine; |
| 204. | N-benzyl-1-(1-butyl-2-phenyl-1H-imidazol-5-yl)-N-(4-methoxybenzyl)methanamine; |
| 205. | N-benzyl-1-(1-butyl-2-phenyl-1H-imidazol-5-yl)-N-[(6-chloro-1,3-benzodioxol-5- |
| | yl)methyl]methanamine; |
| 206. | N-[(1-butyl-2-phenyl-1H-imidazol-5-yl)methyl]-N-(2,3-dichlorobenzyl)butan-1-amine; |
| 207. | 1-(1,3-benzodioxol-5-yl)-N-benzyl-N-[(1-butyl-2-phenyl-1H-imidazol-4- |
| | yl)methyl]methanamine; |
| 208. | 1-(1,3-benzodioxol-5-yl)-N-benzyl-N-[(1-isopentyl-2-phenyl-1H-imidazol-4- |
| | yl)methyl]methanamine; |
| 209. | N-benzyl-1-(1-butyl-2-phenyl-1H-imidazol-5-yl)-N-(3-methoxybenzyl)methanamine; |
| 210. | N-benzyl-1-(1-butyl-2-phenyl-1H-imidazol-5-yl)-N-(2-chloro-4- |
| | fluorobenzyl)methanamine; |
| 211. | N-benzyl-1-(1-butyl-2-phenyl-1H-imidazol-5-yl)-N-(2,6-dichlorobenzyl)methanamine; |
| 212. | 4-({benzyl[(1-butyl-2-phenyl-1H-imidazol-5-yl)methyl]amino}methyl)-3-chlorophenol; |
| 213. | N-benzyl-1-(1-butyl-2-phenyl-1H-imidazol-5-yl)-N-(4-pyrrolidin-1- |
| | ylbenzyl)methanamine; |
| 214. | N-benzyl-N-[(1-butyl-2-phenyl-1H-imidazol-5-yl)methyl]-N-[4- |
| | (diethylamino)benzyl]amine; |
| 215. | N-benzyl-1-(1-butyl-2-phenyl-1H-imidazol-5-yl)-N-(pyridin-2-ylmethyl)methanamine; |
| 216. | 1-(1,3-benzodioxol-5-yl)-N-[(1-butyl-2-phenyl-1H-imidazol-5-yl)methyl]-N-(quinolin- |
| | 6-ylmethyl)methanamine; |
| 217. | N-benzyl-1-(1-butyl-2-phenyl-1H-imidazol-5-yl)-N-(2-chloro-6- |
| | fluorobenzyl)methanamine; |
| 218. | N-benzyl-1-(1-butyl-2-phenyl-1H-imidazol-5-yl)-N-(2,4-dichlorobenzyl)methanamine; |
| 219. | N-benzyl-1-(1-butyl-2-phenyl-1H-imidazol-5-yl)-N-(4-chlorobenzyl)methanamine; |
| 220. | 4-({benzyl[(1-butyl-2-phenyl-1H-imidazol-5-yl)methyl]amino}methyl)phenol; |
| 221. | N-benzyl-1-(1-butyl-2-phenyl-1H-imidazol-5-yl)-N-[4- |
| | (trifluoromethoxy)benzyl]methanamine; |
| 222. | N-benzyl-1-(1-butyl-2-phenyl-1H-imidazol-5-yl)-N-(2-chloro-3,4- |
| | dimethoxybenzyl)methanamine; |
| 223. | N-benzyl-1-(1-butyl-2-phenyl-1H-imidazol-5-yl)-N-(4-nitrobenzyl)methanamine; |
| 224. | N-(4-aminobenzyl)-N-benzyl-N-[(1-butyl-2-phenyl-1H-imidazol-5-yl)methyl]amine; |
| 225. | 1-(1,3-benzodioxol-5-yl)-N-[(1-butyl-2-phenyl-1H-imidazol-5-yl)methyl]-N-(2,3- |
| | dichlorobenzyl)methanamine; |
| 226. | N-(1,3-benzodioxol-5-ylmethyl)-N-[(1-butyl-2-phenyl-1H-imidazol-5-yl)methyl]indan- |
| | 2-amine; |
| 227. | N-(1,3-benzodioxol-5-ylmethyl)-N-[(1-butyl-2-phenyl-1H-imidazol-5-yl)methyl]-2,2- |
| | dimethylpropan-1-amine; |
| 228. | N-(1,3-benzodioxol-5-ylmethyl)-N-[(1-butyl-2-phenyl-1H-imidazol-5-yl)methyl]-3- |
| | methylbutan-1-amine; |
| 229. | 5-({benzyl[(1-butyl-2-phenyl-1H-imidazol-5-yl)methyl]amino}methyl)pyridin-2-amine; |
| 230. | N-benzyl-1-(1-butyl-2-phenyl-1H-imidazol-5-yl)-N-(2,3-dihydro-1-benzofuran-5- |
| | ylmethyl)methanamine; |
| 231. | 4-({butyl[(1-butyl-2-phenyl-1H-imidazol-5-yl)methyl]amino}methyl)-3-chlorophenol |
| 232. | N-[(1-butyl-2-phenyl-1H-imidazol-5-yl)methyl]-N-[4-(dimethylamino)benzyl]-N- |
| | isopentylamine; |
| 233. | 4-[(benzyl{[1-butyl-2-(4-fluorophenyl)-1H-imidazol-5-yl]methyl}amino)methyl]-3- |
| | chlorophenol; |
| 234. | 4-[(benzyl{[1-butyl-2-(3-fluorophenyl)-1H-imidazol-5-yl]methyl}amino)methyl]-3- |
| | chlorophenol; |
| 235. | N-benzyl-1-[1-butyl-2-(3-fluorophenyl)-1H-imidazol-5-yl]-N-(2,3- |
| | dichlorobenzyl)methanamine; |
| 236. | 4-[(benzyl{[1-butyl-2-(3-fluorophenyl)-1H-imidazol-5-yl]methyl}amino)methyl]-N,N- |
| | dimethylaniline; |
| 237. | N-benzyl-1-[1-butyl-2-(3-fluorophenyl)-1H-imidazol-5-yl]-N-(4-pyrrolidin-1- |
| | ylbenzyl)methanamine; |
| 238. | 1-(1,3-benzodioxol-5-yl)-N-benzyl-N-{[1-butyl-2-(4-chlorophenyl)-1H-imidazol-5- |
| | yl]methyl}methanamine; |
| 239. | N-benzyl-1-[1-butyl-2-(4-chlorophenyl)-1H-imidazol-5-yl]-N-(2,3-dihydro-1,4- |
| | benzodioxin-6-ylmethyl)methanamine; |
| 240. | N-(1,3-benzodioxol-5-ylmethyl)-N-benzyl-1-(1-butyl-2-phenyl-1H-imidazol-5- |
| | yl)ethanamine; |
| 241. | 1-(1,3-benzodioxol-5-yl)-N-benzyl-N-[(1-butyl-2-cyclohexyl-1H-imidazol-5- |
| | yl)methyl]methanamine; |
| 242. | N-benzyl-1-(1-butyl-2-cyclohexyl-1H-imidazol-5-yl)-N-(2,3-dihydro-1,4-benzodioxin- |
| | 6-ylmethyl)methanamine; |
| 243. | N-benzyl-1-(1-butyl-2-cyclohexyl-1H-imidazol-5-yl)-N-(1- |
| | naphthylmethyl)methanamine; |
| 244. | N-benzyl-1-(1-butyl-2-cyclohexyl-1H-imidazol-5-yl)-N-(2- |
| | naphthylmethyl)methanamine; |
| 245. | 1-(1,3-benzodioxol-5-yl)-N-(1,3-benzodioxol-5-ylmethyl)-N-[(1-butyl-2-cyclohexyl- |
| | 1H-imidazol-5-yl)methyl]methanamine; |
| 246. | N-benzyl-1-(1-butyl-2-cyclohexyl-1H-imidazol-5-yl)-N-(2,3- |
| | dichlorobenzyl)methanamine; |
| 247. | N-(1,3-benzodioxol-5-ylmethyl)-N-[(1-butyl-2-phenyl-1H-imidazol-5-yl)methyl]-2- |
| | cyclopentylethanamine; |
| 248. | N-(1,3-benzodioxol-5-ylmethyl)-N-[(1-butyl-2-phenyl-1H-imidazol-5-yl)methyl]-2,2- |
| | dimethylbutan-1-amine; |
| 249. | 1-(1,3-benzodioxol-5-yl)-N-[(1-butyl-2-phenyl-1H-imidazol-5-yl)methyl]-N-(3,4- |
| | dimethylbenzyl)methanamine; |
| 250. | N-(1,3-benzodioxol-5-ylmethyl)-N-[(1-butyl-2-phenyl-1H-imidazol-5-yl)methyl]octan- |
| | 1-amine; |
| 251. | 1-(1,3-benzodioxol-5-yl)-N-[(1-butyl-2-phenyl-1H-imidazol-5-yl)methyl]-N- |
| | (cycloheptylmethyl)methanamine; |
| 252. | 1-(1,3-benzodioxol-5-yl)-N-[(1-butyl-2-phenyl-1H-imidazol-5-yl)methyl]-N- |
| | (cyclopropylmethyl)methanamine; |
| 253. | (1R)-N-benzyl-N-[(1-butyl-2-phenyl-1H-imidazol-5-yl)methyl]-1-phenylethanamine; |
| 254. | N-(1,3-benzodioxol-5-ylmethyl)-N-[(1-butyl-2-phenyl-1H-imidazol-5-yl)methyl]-1- |
| | phenylethanamine; |
| 255. | 2-[(1-butyl-2-phenyl-1H-imidazol-5-yl)methyl]-1-cyclopentyl-1,2,3,4- |
| | tetrahydroisoquinoline; |
| 256. | 1-(1,3-benzodioxol-5-yl)-N-benzyl-N[(1-isopentyl-2-phenyl-1H-imidazol-5- |
| | yl)methyl]methanamine; |
| 257. | 4-{[{[1-butyl-2-(3-fluorophenyl)-1H-imidazol-5-yl]methyl}(isopentyl)amino]methyl}- |
| | N,N-dimethylaniline |
| 258. | N-benzyl-1-[1-butyl-2-(3-fluorophenyl)-1H-imidazol-5-yl]-N-(2,3-dihydro-1- |
| | benzofuran-5-ylmethyl)methanamine; |
| 259. | N-benzyl-1-[1-butyl-2-(3-fluorophenyl)-1H-imidazol-5-yl]-N-(2,3-dihydro-1- |
| | benzofuran-5-ylmethyl)ethanamine |
| 260. | 4-[(benzyl{1-[1-butyl-2-(3-fluorophenyl)-1H-imidazol-5-yl]ethyl}amino)methyl]-N,N- |
| | diethylaniline; |
| 261. | N-benzyl-1-[1-butyl-2-(3-fluorophenyl)-1H-imidazol-5-yl]-N-(4-pyrrolidin-1- |
| | ylbenzyl)ethanamine; |
| 262. | 4-[(benzyl{1-[1-butyl-2-(3-fluorophenyl)-1H-imidazol-5-yl]ethyl}amino)methyl]-3- |
| | chlorophenol; |
| 263. | 1-(1,3-benzodioxol-5-yl)-N-[(1-butyl-2-phenyl-1H-imidazol-5-yl)methyl]-N-(2,3- |
| | dihydro-1-benzofuran-5-ylmethyl)methanamine; |
| 264. | N,N-bis(1,3-benzodioxol-5-ylmethyl)-1-[1-butyl-2-(4-fluorophenyl)-1H-imidazol-5- |
| | yl]ethanamine; |
| 265. | N-(1,3-benzodioxol-5-ylmethyl)-N-benzyl-1-[1-butyl-2-(4-fluorophenyl)-1H-imidazol- |
| | 5-yl]ethanamine; |
| 266. | N-(1,3-benzodioxol-5-ylmethyl)-1-[1-butyl-2-(4-fluorophenyl)-1H-imidazol-5-yl]-N- |
| | (cyclopentylmethyl)ethanamine; |
| 267. | 1-(1,3-benzodioxol-5-yl)-N-(1,3-benzodioxol-5-ylmethyl)-N-[(1-butyl-2-thien-2-yl-1H- |
| | imidazol-5-yl)methyl]methanamine; |
| 268. | 1-(1,3-benzodioxol-5-yl)-N-benzyl-N-[(1-butyl-2-thien-2-yl-1H-imidazol-5- |
| | yl)methyl]methanamine; |
| 269. | 1-(1,3-benzodioxol-5-yl)-N-[(1-butyl-2-thien-2-yl-1H-imidazol-5-yl)methyl]-N- |
| | (cyclopentylmethyl)methanamine; |
| 270. | 1-(1,3-benzodioxol-5-yl)-N-[(1-butyl-2-phenyl-1H-imidazol-5-yl)methyl]-N-[(1- |
| | methyl-1H-indol-5-yl)methyl]methanamine; |
| 271. | 4-({(1,3-benzodioxol-5-ylmethyl)[(1-butyl-2-phenyl-1H-imidazol-5- |
| | yl)methyl]amino}methyl)-3-chlorophenol; |
| 272. | 4-[((1,3-benzodioxol-5-ylmethyl){1-[1-butyl-2-(4-fluorophenyl)-1H-imidazol-5- |
| | yl]ethyl}amino)methyl]-3-chlorophenol; |
| 273. | N-benzyl-1-[1-butyl-2-(3-fluorophenyl)-1H-imidazol-5-yl]-N-(2-chloro-4- |
| | methoxybenzyl)methanamine; |
| 274. | 4-{[{[1-butyl-2-(3-fluorophenyl)-1H-imidazol-5-yl]methyl}(isopentyl)amino]methyl}- |
| | 3-chlorophenol; |
| 275. | 4-{[{1-[1-butyl-2-(3-fluorophenyl)-1H-imidazol-5-yl]ethyl}(isopentyl)amino]methyl}- |
| | 3-chlorophenol; |
| 276. | N-{[1-butyl-2-(3-fluorophenyl)-1H-imidazol-5-yl]methyl}-N-(2,3-dihydro-1- |
| | benzofuran-5-ylmethyl)-3-methylbutan-1-amine; |
| 277. | 4-[(butyl{[1-butyl-2-(3-fluorophenyl)-1H-imidazol-5-yl]methyl}amino)methyl]-3- |
| | chlorophenol; |
| 278. | 4-[(butyl{1-[1-butyl-2-(3-fluorophenyl)-1H-imidazol-5-yl]ethyl}amino)methyl]-3- |
| | chlorophenol; |
| 279. | N-{1-[1-butyl-2-(3-fluorophenyl)-1H-imidazol-5-yl]ethyl}-N-(2,3- |
| | dichlorobenzyl)butan-1-amine; |
| 280. | 1-(1-butyl-2-phenyl-1H-imidazol-5-yl)-N,N-bis(2,3-dihydro-1-benzofuran-5- |
| | ylmethyl)methanamine; |
| 281. | 1-(1,3-benzodioxol-5-yl)-N-benzyl-N-{[1-butyl-2-(3-fluoro-2-methylphenyl)-1H- |
| | imidazol-5-yl]methyl}methanamine hydrofluoride; |
| 282. | 4-[(benzyl{(1R)-1-[1-butyl-2-(3-fluorophenyl)-1H-imidazol-5-yl]ethyl}amino)methyl]- |
| | 3-chlorophenol; |
| 283. | N,N-bis(1,3-benzodioxol-5-ylmethyl)-1-(1-butyl-2-phenyl-1H-imidazol-5- |
| | yl)ethanamine; |
| 284. | N-(1,3-benzodioxol-5-ylmethyl)-1-(1-butyl-2-phenyl-1H-imidazol-5-yl)-N- |
| | (cyclohexylmethyl)pentan-1-amine; |
| 285. | 4-({(1,3-benzodioxol-5-ylmethyl)[1-(1-butyl-2-phenyl-1H-imidazol-5- |
| | yl)ethyl]amino}methyl)-3-chlorophenol; |
| 286. | 4-{[[1-(1-butyl-2-phenyl-1H-imidazol-5-yl)pentyl](cyclohexylmethyl)amino]methyl}- |
| | 3-chlorophenol; |
| 287. | 4-({(1,3-benzodioxol-5-ylmethyl)[1-(1-butyl-2-phenyl-1H-imidazol-5- |
| | yl)ethyl]amino}methyl)phenol; |
| 288. | 4-{[[1-(1-butyl-2-phenyl-1H-imidazol-5- |
| | yl)pentyl](cyclohexylmethyl)amino]methyl}phenol; |
| 289. | 4-{[[1-(1-butyl-2-phenyl-1H-imidazol-5-yl)pentyl](cyclohexylmethyl)amino] |
| | methyl}benzoic acid; |
| 290. | methyl 4-({benzyl[(1-butyl-2-phenyl-1H-imidazol-5- |
| | yl)methyl]amino}methyl)benzoate; |
| 291. | methyl 4-({butyl[(1-butyl-2-phenyl-1H-imidazol-5-yl)methyl]amino}methyl)benzoate; |
| 292. | methyl 4-{[[(1-butyl-2-phenyl-1H-imidazol-5-yl)methyl](cyclohexylmethyl)amino] |
| | methyl}benzoate; |
| 293. | 4-{[[(1-butyl-2-phenyl-1H-imidazol-5-yl)methyl](cyclohexylmethyl)amino] |
| | methyl}benzoic acid; |
| 294. | (4-{[[1-(1-butyl-2-phenyl-1H-imidazol-5-yl)pentyl](cyclohexylmethyl)amino]methyl} |
| | phenyl)methanol; |
| 295. | [4-({benzyl[(1-butyl-2-phenyl-1H-imidazol-5-yl)methyl]amino}methyl)phenyl] |
| | methanol; |
| 296. | [4-({butyl[(1-butyl-2-phenyl-1H-imidazol-5-yl)methyl]amino}methyl)phenyl]methanol; |
| 297. | (4-{[[(1-butyl-2-phenyl-1H-imidazol-5-yl)methyl](cyclohexylmethyl)amino]methyl} |
| | phenyl)methanol; |
| 298. | methyl 3-({benzyl[1-(1-butyl-2-phenyl-1H-imidazol-5-yl)pentyl]amino}methyl) |
| | benzoate; |
| 299. | methyl 3-({butyl[1-(1-butyl-2-phenyl-1H-imidazol-5- |
| | yl)pentyl]amino}methyl)benzoate; |
| 300. | methyl 3-{[[1-(1-butyl-2-phenyl-1H-imidazol-5- |
| | yl)pentyl](cyclohexylmethyl)amino]methyl}benzoate; |
| 301. | 3-({benzyl[1-(1-butyl-2-phenyl-1H-imidazol-5-yl)pentyl]amino}methyl)benzoic acid; |
| 302. | 3-{[[1-(1-butyl-2-phenyl-1H-imidazol-5-yl)pentyl](cyclohexylmethyl)amino]methyl} |
| | benzoic acid; |
| 303. | [3-({benzyl[1-(1-butyl-2-phenyl-1H-imidazol-5-yl)pentyl]amino}methyl)phenyl] |
| | methanol; |
| 304. | [3-({butyl[1-(1-butyl-2-phenyl-1H-imidazol-5- |
| | yl)pentyl]amino}methyl)phenyl]methanol; |
| 305. | (3-{[[1-(1-butyl-2-phenyl-1H-imidazol-5-yl)pentyl](cyclohexylmethyl)amino]methyl} |
| | phenyl)methanol; |
| 306. | methyl 5-({benzyl[1-(1-butyl-2-phenyl-1H-imidazol-5-yl)pentyl]amino}methyl)-2- |
| | hydroxybenzoate; |
| 307. | methyl 5-({butyl[1-(1-butyl-2-phenyl-1H-imidazol-5-yl)pentyl]amino}methyl)-2- |
| | hydroxybenzoate; |
| 308. | methyl 5-{[[1-(1-butyl-2-phenyl-1H-imidazol-5-yl)pentyl](cyclohexylmethyl)amino] |
| | methyl}-2-hydroxybenzoate; |
| 309. | methyl 5-({benzyl[(1-butyl-2-phenyl-1H-imidazol-5-yl)methyl]amino}methyl)-2- |
| | hydroxybenzoate; |
| 310. | methyl 5-({butyl[(1-butyl-2-phenyl-1H-imidazol-5-yl)methyl]amino}methyl)-2- |
| | hydroxybenzoate; |
| 311. | methyl 5-{[[(1-butyl-2-phenyl-1H-imidazol-5-yl)methyl](cyclohexylmethyl)amino] |
| | methyl}-2-hydroxybenzoate; |
| 312. | 5-({benzyl[1-(1-butyl-2-phenyl-1H-imidazol-5-yl)pentyl]amino}methyl)-2- |
| | hydroxybenzoic acid; |
| 313. | 5-{[[1-(1-butyl-2-phenyl-1H-imidazol-5-yl)pentyl](cyclohexylmethyl)amino]methyl}- |
| | 2-hydroxybenzoic acid; |
| 314. | 5-({benzyl[(1-butyl-2-phenyl-1H-imidazol-5-yl)methyl]amino}methyl)-2- |
| | hydroxybenzoic acid; |
| 315. | 5-({butyl[(1-butyl-2-phenyl-1H-imidazol-5-yl)methyl]amino}methyl)-2- |
| | hydroxybenzoic acid; |
| 316. | 5-{[[(1-butyl-2-phenyl-1H-imidazol-5-yl)methyl](cyclohexylmethyl)amino]methyl}-2- |
| | hydroxybenzoic acid; |
| 317. | 4-({benzyl[1-(1-butyl-2-phenyl-1H-imidazol-5-yl)pentyl]amino}methyl)-2- |
| | (hydroxymethyl)phenol; |
| 318. | 4-({butyl[1-(1-butyl-2-phenyl-1H-imidazol-5-yl)pentyl]amino}methyl)-2- |
| | (hydroxymethyl)phenol; |
| 319. | 4-{[[1-(1-butyl-2-phenyl-1H-imidazol-5-yl)pentyl](cyclohexylmethyl)amino]methyl}- |
| | 2-(hydroxymethyl)phenol; |
| 320. | methyl 5-({benzyl[1-(1-butyl-2-phenyl-1H-imidazol-5-yl)pentyl]amino}methyl)-2- |
| | methoxybenzoate; |
| 321. | methyl 5-({butyl[1-(1-butyl-2-phenyl-1H-imidazol-5-yl)pentyl]amino}methyl)-2- |
| | methoxybenzoate; |
| 322. | methyl 5-{[[1-(1-butyl-2-phenyl-1H-imidazol-5-yl)pentyl](cyclohexylmethyl)amino] |
| | methyl}-2-methoxybenzoate; |
| 323. | methyl 5-({benzyl[(1-butyl-2-phenyl-1H-imidazol-5-yl)methyl]amino}methyl)-2- |
| | methoxybenzoate; |
| 324. | methyl 5-{[[(1-butyl-2-phenyl-1H-imidazol-5-yl)methyl](cyclohexylmethyl)amino] |
| | methyl}-2-methoxybenzoate; |
| 325. | 5-{[[1-(1-butyl-2-phenyl-1H-imidazol-5-yl)pentyl](cyclohexylmethyl)amino]methyl}- |
| | 2-methoxybenzoic acid; |
| 326. | [5-({benzyl[1-(1-butyl-2-phenyl-1H-imidazol-5-yl)pentyl]amino}methyl)-2- |
| | methoxyphenyl]methanol; |
| 327. | [5-({butyl[1-(1-butyl-2-phenyl-1H-imidazol-5-yl)pentyl]amino}methyl)-2- |
| | methoxyphenyl]methanol; |
| 328. | (5-{[[1-(1-butyl-2-phenyl-1H-imidazol-5-yl)pentyl](cyclohexylmethyl)amino]methyl}- |
| | 2-methoxyphenyl)methanol; |
| 329. | [5-({benzyl[(1-butyl-2-phenyl-1H-imidazol-5-yl)methyl]amino}methyl)-2- |
| | methoxyphenyl]methanol; |
| 330. | (5-{[[(1-butyl-2-phenyl-1H-imidazol-5-yl)methyl](cyclohexylmethyl)amino]methyl}-2- |
| | methoxyphenyl)methanol; |
| 331. | methyl 4-{[[(1-butyl-2-phenyl-1H-imidazol-5-yl)methyl](4- |
| | hydroxybenzyl)amino]methyl}benzoate; |
| 332. | methyl 4-{[[(1-butyl-2-phenyl-1H-imidazol-5-yl)methyl](2-chloro-4- |
| | hydroxybenzyl)amino]methyl}benzoate; |
| 333. | methyl 4-{[[(1-butyl-2-phenyl-1H-imidazol-5-yl)methyl](4- |
| | methoxybenzyl)amino]methyl}benzoate; |
| 334. | methyl 4-({(1,3-benzodioxol-5-ylmethyl)[(1-butyl-2-phenyl-1H-imidazol-5- |
| | yl)methyl]amino}methyl)benzoate; |
| 335. | diethyl 5-({benzyl[(1-butyl-2-phenyl-1H-imidazol-5-yl)methyl]amino} |
| | methyl)isophthalate; |
| 336. | diethyl 5-({butyl[(1-butyl-2-phenyl-1H-imidazol-5- |
| | yl)methyl]amino}methyl)isophthalate; |
| 337. | diethyl 5-{[[(1-butyl-2-phenyl-1H-imidazol-5-yl)methyl](cyclohexylmethyl)amino] |
| | methyl}isophthalate; |
| 338. | 4-{[[(1-butyl-2-phenyl-1H-imidazol-5-yl)methyl](4-hydroxybenzyl)amino] |
| | methyl}benzoic acid; |
| 339. | 4-{[[(1-butyl-2-phenyl-1H-imidazol-5-yl)methyl](2-chloro-4-hydroxybenzyl)amino] |
| | methyl}benzoic acid; |
| 340. | 4-{[[(1-butyl-2-phenyl-1H-imidazol-5-yl)methyl](4-methoxybenzyl)amino] |
| | methyl}benzoic acid; |
| 341. | 4-({(1,3-benzodioxol-5-ylmethyl)[(1-butyl-2-phenyl-1H-imidazol-5-yl)methyl]amino} |
| | methyl)benzoic acid; |
| 342. | 4-({[(1-butyl-2-phenyl-1H-imidazol-5-yl)methyl][4-(hydroxymethyl)benzyl] |
| | amino}methyl)phenol; |
| 343. | 4-({[(1-butyl-2-phenyl-1H-imidazol-5-yl)methyl][4-(hydroxymethyl)benzyl] |
| | amino}methyl)-3-chlorophenol; |
| 344. | (4-{[[(1-butyl-2-phenyl-1H-imidazol-5-yl)methyl](4-methoxybenzyl)amino] |
| | methyl}phenyl)methanol; |
| 345. | [4-({(1,3-benzodioxol-5-ylmethyl)[(1-butyl-2-phenyl-1H-imidazol-5-yl)methyl]amino} |
| | methyl)phenyl]methanol; |
| 346. | N-(1,3-benzodioxol-5-ylmethyl)-N-benzyl-1-(1-butyl-2-phenyl-1H-imidazol-5- |
| | yl)pentan-1-amine; |
| 347. | 4-({benzyl[1-(1-butyl-2-phenyl-1H-imidazol-5-yl)pentyl]amino}methyl)phenol; |
| 348. | 4-({benzyl[1-(1-butyl-2-phenyl-1H-imidazol-5-yl)pentyl]amino}methyl)-3- |
| | chlorophenol; |
| 349. | 3-({benzyl[1-(1-butyl-2-phenyl-1H-imidazol-5-yl)pentyl]amino}methyl)phenol; |
| 350. | 4-({benzyl[1-(1-butyl-2-phenyl-1H-imidazol-5-yl)pentyl]amino}methyl)phenyl acetate; |
| 351. | 3-({benzyl[1-(1-butyl-2-phenyl-1H-imidazol-5-yl)pentyl]amino}methyl)phenyl acetate; |
| 352. | N-(1,3-benzodioxol-5-ylmethyl)-N-butyl-1-(1-butyl-2-phenyl-1H-imidazol-5-yl)pentan- |
| | 1-amine; |
| 353. | 4-({butyl[1-(1-butyl-2-phenyl-1H-imidazol-5-yl)pentyl]amino}methyl)phenol; |
| 354. | 4-({butyl[1-(1-butyl-2-phenyl-1H-imidazol-5-yl)pentyl]amino}methyl)-3-chlorophenol; |
| 355. | 3-({butyl[1-(1-butyl-2-phenyl-1H-imidazol-5-yl)pentyl]amino}methyl)phenol; |
| 356. | 4-({butyl [1-(1-butyl-2-phenyl-1H-imidazol-5-yl)pentyl]amino}methyl)phenyl acetate; |
| 357. | 3-({butyl[1-(1-butyl-2-phenyl-1H-imidazol-5-yl)pentyl]amino}methyl)phenyl acetate; |
| 358. | N-benzyl-1-(1-butyl-2-phenyl-1H-imidazol-5-yl)-N-(4-methoxybenzyl)pentan-1-amine; |
| 359. | N-butyl-1-(1-butyl-2-phenyl-1H-imidazol-5-yl)-N-(4-methoxybenzyl)pentan-1-amine; |
| 360. | 1-(1-butyl-2-phenyl-1H-imidazol-5-yl)-N-(cyclohexylmethyl)-N-(4- |
| | methoxybenzyl)pentan-1-amine; |
| 361. | N-butyl-1-(1-butyl-2-phenyl-1H-imidazol-5-yl)-N-[4-(trifluoromethoxy)benzyl]pentan- |
| | 1-amine; |
| 362. | N-benzyl-1-(1-butyl-2-phenyl-1H-imidazol-5-yl)-N-(3-methoxybenzyl)pentan-1-amine; |
| 363. | N-butyl-1-(1-butyl-2-phenyl-1H-imidazol-5-yl)-N-(3-methoxybenzyl)pentan-1-amine; |
| 364. | 1-(1-butyl-2-phenyl-1H-imidazol-5-yl)-N-(cyclohexylmethyl)-N-(3- |
| | methoxybenzyl)pentan-1-amine; |
| 365. | 4-({benzyl[1-(1-butyl-2-phenyl-1H-imidazol-5-yl)pentyl]amino}methyl)-N,N- |
| | dimethylaniline; |
| 366. | 4-({butyl[1-(1-butyl-2-phenyl-1H-imidazol-5-yl)pentyl]amino}methyl)-N,N- |
| | dimethylaniline; |
| 367. | 4-{[[1-(1-butyl-2-phenyl-1H-imidazol-5-yl)pentyl](cyclohexylmethyl)amino]methyl}- |
| | N,N-dimethylaniline; |
| 368. | (1R)-N-(1,3-benzodioxol-5-ylmethyl)-1-(1-butyl-2-phenyl-1H-imidazol-5-yl)-N- |
| | (cyclohexylmethyl)pentan-1-amine; |
| 369. | 4-{[[(1R)-1-(1-butyl-2-phenyl-1H-imidazol-5-yl)pentyl](cyclohexylmethyl)amino] |
| | methyl}-3-chlorophenol; |
| 370. | (1R)-1-(1-butyl-2-phenyl-1H-imidazol-5-yl)-N-(cyclohexylmethyl)-N-(4- |
| | methoxybenzyl)butan-1-amine. |
| 371. | N,N-dibenzyl-1-[1-butyl-2-(2-fluorophenyl)-1H-imidazol-5-yl]methanamine |
| 372. | N-benzyl-1-[1-butyl-2-(2-fluorophenyl)-1H-imidazol-5-yl]-N-(3- |
| | methylbenzyl)methanamine |
| 373. | N-benzyl-1-[1-butyl-2-(2-fluorophenyl)-1H-imidazol-5-yl]-N-(4- |
| | ethylbenzyl)methanamine |
| 374. | N-benzyl-1-[1-butyl-2-(4-fluorophenyl)-1H-imidazol-5-yl]-N-(3,4- |
| | dimethylbenzyl)methanamine |
| 375. | N-benzyl-1-[1-butyl-2-(2-fluorophenyl)-1H-imidazol-5-yl]-N-(4- |
| | isopropylbenzyl)methanamine |
| 376. | N-(1,3-benzodioxol-5-ylmethyl)-N-{[1-butyl-2-(4-fluorophenyl)-1H-imidazol-5- |
| | yl]methyl}-2-phenylethanamine |
| 377. | N-benzyl-1-[1-butyl-2-(4-methoxyphenyl)-1H-imidazol-5-yl]-N-(4- |
| | isopropylbenzyl)methanamine |
| 378. | N-(1,3-benzodioxol-5-ylmethyl)-N-[(1-butyl-2-phenyl-1H-imidazol-5-yl)methyl]butan- |
| | 1-amine |
| 379. | N-(1,3-benzodioxol-5-ylmethyl)-N-[(1-butyl-2-phenyl-1H-imidazol-5-yl)methyl]-2- |
| | methylpropan-1-amine |
| 380. | 1-(1,3-benzodioxol-5-yl)-N-[(1-butyl-2-phenyl-1H-imidazol-5-yl)methyl]-N- |
| | (cyclobutylmethyl)methanamine |
| 381. | N-(1,3-benzodioxol-5-ylmethyl)-N-[(1-butyl-2-phenyl-1H-imidazol-5- |
| | yl)methyl]pentan-1-amine |
| 382. | N-(1,3-benzodioxol-5-ylmethyl)-N-[(1-butyl-2-phenyl-1H-imidazol-5-yl)methyl]-3- |
| | methylbutan-1-amine |
| 383. | N-(1,3-benzodioxol-5-ylmethyl)-N-[(1-butyl-2-phenyl-1H-imidazol-5-yl)methyl]-2,2- |
| | dimethylpropan-1-amine |
| 384. | N-benzyl-1-(1-butyl-2-phenyl-1H-imidazol-5-yl)-N-(cyclopentylmethyl)methanamine |
| 385. | 1-(1,3-benzodioxol-5-yl)-N-[(1-butyl-2-phenyl-1H-imidazol-5-yl)methyl]-N- |
| | (cyclopentylmethyl)methanamine |
| 386. | N-(1,3-benzodioxol-5-ylmethyl)-N-[(1-butyl-2-phenyl-1H-imidazol-5-yl)methyl]hexan- |
| | 1-amine |
| 387. | N-(1,3-benzodioxol-5-ylmethyl)-N-[(1-butyl-2-phenyl-1H-imidazol-5-yl)methyl]-4- |
| | methylpentan-1-amine |
| 388. | N-(1,3-benzodioxol-5-ylmethyl)-N-[(1-butyl-2-phenyl-1H-imidazol-5-yl)methyl]-3,3- |
| | dimethylbutan-1-amine |
| 389. | N-(1,3-benzodioxol-5-ylmethyl)-N-[(1-butyl-2-phenyl-1H-imidazol-5-yl)methyl]-2- |
| | ethylbutan-1-amine |
| 390. | N-benzyl-N-[(1-butyl-2-phenyl-1H-imidazol-5-yl)methyl]-2,2-dimethylbutan-1-amine |
| 391. | N-(1,3-benzodioxol-5-ylmethyl)-N-[(1-butyl-2-phenyl-1H-imidazol-5-yl)methyl]-2,2- |
| | dimethylbutan-1-amine |
| 392. | N-(1,3-benzodioxol-5-ylmethyl)-N-[(1-butyl-2-phenyl-1H-imidazol-5-yl)methyl]-2- |
| | cyclopentylethanamine |
| 393. | 1-(1,3-benzodioxol-5-yl)-N-[(1-butyl-2-phenyl-1H-imidazol-5-yl)methyl]-N- |
| | (cyclohexylmethyl)methanamine |
| 394. | N-(1,3-benzodioxol-5-ylmethyl)-N-[(1-butyl-2-phenyl-1H-imidazol-5- |
| | yl)methyl]heptan-1-amine |
| 395. | N-(1,3-benzodioxol-5-ylmethyl)-N-[(1-butyl-2-phenyl-1H-imidazol-5-yl)methyl]-2- |
| | cyclohexylethanamine |
| 396. | 1-(1,3-benzodioxol-5-yl)-N-[(1-butyl-2-phenyl-1H-imidazol-5-yl)methyl]-N- |
| | (cycloheptylmethyl)methanamine |
| 397. | N-(1,3-benzodioxol-5-ylmethyl)-N-[(1-butyl-2-phenyl-1H-imidazol-5-yl)methyl]-3- |
| | cyclopentylpropan-1-amine |
| 398. | N-(1,3-benzodioxol-5-ylmethyl)-N-[(1-butyl-2-phenyl-1H-imidazol-5-yl)methyl]octan- |
| | 1-amine |
| 399. | 1-(1,3-benzodioxol-5-yl)-N-(1,1′-biphenyl-2-ylmethyl)-N-[(1-butyl-2-phenyl-1H- |
| | imidazol-5-yl)methyl]methanamine |
| 400. | 1-(1,3-benzodioxol-5-yl)-N-[(1-butyl-2-phenyl-1H-imidazol-5-yl)methyl]-N-(2- |
| | phenoxybenzyl)methanamine |
| 401. | N-benzyl-N-[(1-butyl-2-phenyl-1H-imidazol-5-yl)methyl]-2-phenylethanamine |
| 402. | N,N-dibenzyl-1-(1-butyl-2-phenyl-1H-imidazol-5-yl)methanamine |
| 403. | N-benzyl-1-(1-butyl-2-phenyl-1H-imidazol-5-yl)-N-(1-naphthylmethyl)methanamine |
| 404. | 1-(1,3-benzodioxol-5-yl)-N-benzyl-N-[(1-butyl-2-phenyl-1H-imidazol-5- |
| | yl)methyl]methanamine |
| 405. | N-[(1-butyl-2-phenyl-1H-imidazol-5-yl)methyl]-N-(3-methylbenzyl)-2- |
| | phenylethanamine |
| 406. | N-benzyl-1-(1-butyl-2-phenyl-1H-imidazol-5-yl)-N-(3-methylbenzyl)methanamine |
| 407. | 1-(1-butyl-2-phenyl-1H-imidazol-5-yl)-N-(3-methylbenzyl)-N-(1- |
| | naphthylmethyl)methanamine |
| 408. | 1-(1,3-benzodioxol-5-yl)-N-[(1-butyl-2-phenyl-1H-imidazol-5-yl)methyl]-N-(3- |
| | methylbenzyl)methanamine |
| 409. | N-[(1-butyl-2-phenyl-1H-imidazol-5-yl)methyl]-N-(4-methylbenzyl)-2- |
| | phenylethanamine |
| 410. | N-benzyl-1-(1-butyl-2-phenyl-1H-imidazol-5-yl)-N-(4-methylbenzyl)methanamine |
| 411. | 1-(1-butyl-2-phenyl-1H-imidazol-5-yl)-N-(4-methylbenzyl)-N-(1- |
| | naphthylmethyl)methanamine |
| 412. | 1-(1,3-benzodioxol-5-yl)-N-[(1-butyl-2-phenyl-1H-imidazol-5-yl)methyl]-N-(4- |
| | methylbenzyl)methanamine |
| 413. | N-benzyl-1-(1-butyl-2-phenyl-1H-imidazol-5-yl)-N-(2-methylbenzyl)methanamine |
| 414. | 1-(1-butyl-2-phenyl-1H-imidazol-5-yl)-N-(2-methylbenzyl)-N-(1- |
| | naphthylmethyl)methanamine |
| 415. | 1-(1,3-benzodioxol-5-yl)-N-[(1-butyl-2-phenyl-1H-imidazol-5-yl)methyl]-N-(2- |
| | methylbenzyl)methanamine |
| 416. | 1-(1,3-benzodioxol-5-yl)-N-[(1-butyl-2-phenyl-1H-imidazol-5-yl)methyl]-N-(3- |
| | fluorobenzyl)methanamine |
| 417. | N-[(1-butyl-2-phenyl-1H-imidazol-5-yl)methyl]-N-(4-fluorobenzyl)-2- |
| | phenylethanamine |
| 418. | 1-(1,3-benzodioxol-5-yl)-N-[(1-butyl-2-phenyl-1H-imidazol-5-yl)methyl]-N-(4- |
| | fluorobenzyl)methanamine |
| 419. | N-[(1-butyl-2-phenyl-1H-imidazol-5-yl)methyl]-N-(2-fluorobenzyl)-2- |
| | phenylethanamine |
| 420. | N-benzyl-1-(1-butyl-2-phenyl-1H-imidazol-5-yl)-N-(2-fluorobenzyl)methanamine |
| 421. | 1-(1-butyl-2-phenyl-1H-imidazol-5-yl)-N-(2-fluorobenzyl)-N-(1- |
| | naphthylmethyl)methanamine |
| 422. | 1-(1,3-benzodioxol-5-yl)-N-[(1-butyl-2-phenyl-1H-imidazol-5-yl)methyl]-N-(2- |
| | fluorobenzyl)methanamine |
| 423. | N-[(1-butyl-2-phenyl-1H-imidazol-5-yl)methyl]-N-(4-ethylbenzyl)-2-phenylethanamine |
| 424. | N-benzyl-1-(1-butyl-2-phenyl-1H-imidazol-5-yl)-N-(4-ethylbenzyl)methanamine |
| 425. | 1-(1-butyl-2-phenyl-1H-imidazol-5-yl)-N-(4-ethylbenzyl)-N-(1- |
| | naphthylmethyl)methanamine |
| 426. | 1-(1,3-benzodioxol-5-yl)-N-[(1-butyl-2-phenyl-1H-imidazol-5-yl)methyl]-N-(4- |
| | ethylbenzyl)methanamine |
| 427. | N-[(1-butyl-2-phenyl-1H-imidazol-5-yl)methyl]-N-(3,4-dimethylbenzyl)-2- |
| | phenylethanamine |
| 428. | N-benzyl-1-(1-butyl-2-phenyl-1H-imidazol-5-yl)-N-(3,4-dimethylbenzyl)methanamine |
| 429. | 1-(1-butyl-2-phenyl-1H-imidazol-5-yl)-N-(3,4-dimethylbenzyl)-N-(1- |
| | naphthylmethyl)methanamine |
| 430. | 1-(1,3-benzodioxol-5-yl)-N-[(1-butyl-2-phenyl-1H-imidazol-5-yl)methyl]-N-(3,4- |
| | dimethylbenzyl)methanamine |
| 431. | N-benzyl-1-(1-butyl-2-phenyl-1H-imidazol-5-yl)-N-(3,5-dimethylbenzyl)methanamine |
| 432. | 1-(1,3-benzodioxol-5-yl)-N-[(1-butyl-2-phenyl-1H-imidazol-5-yl)methyl]-N-(3,5- |
| | dimethylbenzyl)methanamine |
| 433. | N-[(1-butyl-2-phenyl-1H-imidazol-5-yl)methyl]-N-(2,3-dimethylbenzyl)-2- |
| | phenylethanamine |
| 434. | N-benzyl-1-(1-butyl-2-phenyl-1H-imidazol-5-yl)-N-(2,3-dimethylbenzyl)methanamine |
| 435. | 1-(1,3-benzodioxol-5-yl)-N-[(1-butyl-2-phenyl-1H-imidazol-5-yl)methyl]-N-(2,3- |
| | dimethylbenzyl)methanamine |
| 436. | N-[(1-butyl-2-phenyl-1H-imidazol-5-yl)methyl]-N-(2,5-dimethylbenzyl)-2- |
| | phenylethanamine |
| 437. | N-benzyl-1-(1-butyl-2-phenyl-1H-imidazol-5-yl)-N-(2,5-dimethylbenzyl)methanamine |
| 438. | 1-(1-butyl-2-phenyl-1H-imidazol-5-yl)-N-(2,5-dimethylbenzyl)-N-(1- |
| | naphthylmethyl)methanamine |
| 439. | 1-(1,3-benzodioxol-5-yl)-N-[(1-butyl-2-phenyl-1H-imidazol-5-yl)methyl]-N-(2,5- |
| | dimethylbenzyl)methanamine |
| 440. | N-[(1-butyl-2-phenyl-1H-imidazol-5-yl)methyl]-N-(2,4-dimethylbenzyl)-2- |
| | phenylethanamine |
| 441. | N-benzyl-1-(1-butyl-2-phenyl-1H-imidazol-5-yl)-N-(2,4-dimethylbenzyl)methanamine |
| 442. | 1-(1-butyl-2-phenyl-1H-imidazol-5-yl)-N-(2,4-dimethylbenzyl)-N-(1- |
| | naphthylmethyl)methanamine |
| 443. | 1-(1,3-benzodioxol-5-yl)-N-[(1-butyl-2-phenyl-1H-imidazol-5-yl)methyl]-N-(2,4- |
| | dimethylbenzyl)methanamine |
| 444. | N-[(1-butyl-2-phenyl-1H-imidazol-5-yl)methyl]-N-(3-methoxybenzyl)-2- |
| | phenylethanamine |
| 445. | N-benzyl-1-(1-butyl-2-phenyl-1H-imidazol-5-yl)-N-(3-methoxybenzyl)methanamine |
| 446. | 1-(1-butyl-2-phenyl-1H-imidazol-5-yl)-N-(3-methoxybenzyl)-N-(1- |
| | naphthylmethyl)methanamine |
| 447. | 1-(1,3-benzodioxol-5-yl)-N-[(1-butyl-2-phenyl-1H-imidazol-5-yl)methyl]-N-(3- |
| | methoxybenzyl)methanamine |
| 448. | N-[(1-butyl-2-phenyl-1H-imidazol-5-yl)methyl]-N-(4-methoxybenzyl)-2- |
| | phenylethanamine |
| 449. | N-benzyl-1-(1-butyl-2-phenyl-1H-imidazol-5-yl)-N-(4-methoxybenzyl)methanamine |
| 450. | 1-(1-butyl-2-phenyl-1H-imidazol-5-yl)-N-(4-methoxybenzyl)-N-(1- |
| | naphthylmethyl)methanamine |
| 451. | 1-(1,3-benzodioxol-5-yl)-N-[(1-butyl-2-phenyl-1H-imidazol-5-yl)methyl]-N-(4- |
| | methoxybenzyl)methanamine |
| 452. | N-[(1-butyl-2-phenyl-1H-imidazol-5-yl)methyl]-N-(2-methoxybenzyl)-2- |
| | phenylethanamine |
| 453. | N-benzyl-1-(1-butyl-2-phenyl-1H-imidazol-5-yl)-N-(2-methoxybenzyl)methanamine |
| 454. | 1-(1-butyl-2-phenyl-1H-imidazol-5-yl)-N-(2-methoxybenzyl)-N-(1- |
| | naphthylmethyl)methanamine |
| 455. | 1-(1,3-benzodioxol-5-yl)-N-[(1-butyl-2-phenyl-1H-imidazol-5-yl)methyl]-N-(2- |
| | methoxybenzyl)methanamine |
| 456. | N-[(1-butyl-2-phenyl-1H-imidazol-5-yl)methyl]-N-(3-fluoro-4-methylbenzyl)-2- |
| | phenylethanamine |
| 457. | N-benzyl-1-(1-butyl-2-phenyl-1H-imidazol-5-yl)-N-(3-fluoro-4- |
| | methylbenzyl)methanamine |
| 458. | 1-(1-butyl-2-phenyl-1H-imidazol-5-yl)-N-(3-fluoro-4-methylbenzyl)-N-(1- |
| | naphthylmethyl)methanamine |
| 459. | 1-(1,3-benzodioxol-5-yl)-N-[(1-butyl-2-phenyl-1H-imidazol-5-yl)methyl]-N-(3-fluoro- |
| | 4-methylbenzyl)methanamine |
| 460. | N-benzyl-1-(1-butyl-2-phenyl-1H-imidazol-5-yl)-N-(3-fluoro-2- |
| | methylbenzyl)methanamine |
| 461. | 1-(1,3-benzodioxol-5-yl)-N-[(1-butyl-2-phenyl-1H-imidazol-5-yl)methyl]-N-(3-fluoro- |
| | 2-methylbenzyl)methanamine |
| 462. | N-[(1-butyl-2-phenyl-1H-imidazol-5-yl)methyl]-N-(5-fluoro-2-methylbenzyl)-2- |
| | phenylethanamine |
| 463. | N-benzyl-1-(1-butyl-2-phenyl-1H-imidazol-5-yl)-N-(5-fluoro-2- |
| | methylbenzyl)methanamine |
| 464. | 1-(1-butyl-2-phenyl-1H-imidazol-5-yl)-N-(5-fluoro-2-methylbenzyl)-N-(1- |
| | naphthylmethyl)methanamine |
| 465. | 1-(1,3-benzodioxol-5-yl)-N-[(1-butyl-2-phenyl-1H-imidazol-5-yl)methyl]-N-(5-fluoro- |
| | 2-methylbenzyl)methanamine |
| 466. | 1-(1,3-benzodioxol-5-yl)-N-[(1-butyl-2-phenyl-1H-imidazol-5-yl)methyl]-N-(3- |
| | chlorobenzyl)methanamine |
| 467. | 1-(1-butyl-2-phenyl-1H-imidazol-5-yl)-N-(4-chlorobenzyl)-N-(1- |
| | naphthylmethyl)methanamine |
| 468. | 1-(1,3-benzodioxol-5-yl)-N-[(1-butyl-2-phenyl-1H-imidazol-5-yl)methyl]-N-(4- |
| | chlorobenzyl)methanamine |
| 469. | N-[(1-butyl-2-phenyl-1H-imidazol-5-yl)methyl]-N-(2-chlorobenzyl)-2- |
| | phenylethanamine |
| 470. | N-benzyl-1-(1-butyl-2-phenyl-1H-imidazol-5-yl)-N-(2-chlorobenzyl)methanamine |
| 471. | 1-(1-butyl-2-phenyl-1H-imidazol-5-yl)-N-(2-chlorobenzyl)-N-(1- |
| | naphthylmethyl)methanamine |
| 472. | 1-(1,3-benzodioxol-5-yl)-N-[(1-butyl-2-phenyl-1H-imidazol-5-yl)methyl]-N-(2- |
| | chlorobenzyl)methanamine |
| 473. | 1-(1,3-benzodioxol-5-yl)-N-[(1-butyl-2-phenyl-1H-imidazol-5-yl)methyl]-N-(3,4- |
| | difluorobenzyl)methanamine |
| 474. | N-benzyl-1-(1-butyl-2-phenyl-1H-imidazol-5-yl)-N-(2,3-difluorobenzyl)methanamine |
| 475. | 1-(1-butyl-2-phenyl-1H-imidazol-5-yl)-N-(2,3-difluorobenzyl)-N-(1- |
| | naphthylmethyl)methanamine |
| 476. | 1-(1,3-benzodioxol-5-yl)-N-[(1-butyl-2-phenyl-1H-imidazol-5-yl)methyl]-N-(2,3- |
| | difluorobenzyl)methanamine |
| 477. | N-benzyl-1-(1-butyl-2-phenyl-1H-imidazol-5-yl)-N-(2,5-difluorobenzyl)methanamine |
| 478. | 1-(1-butyl-2-phenyl-1H-imidazol-5-yl)-N-(2,5-difluorobenzyl)-N-(1- |
| | naphthylmethyl)methanamine |
| 479. | 1-(1,3-benzodioxol-5-yl)-N-[(1-butyl-2-phenyl-1H-imidazol-5-yl)methyl]-N-(2,5- |
| | difluorobenzyl)methanamine |
| 480. | 1-(1-butyl-2-phenyl-1H-imidazol-5-yl)-N-(2,4-difluorobenzyl)-N-(1- |
| | naphthylmethyl)methanamine |
| 481. | 1-(1,3-benzodioxol-5-yl)-N-[(1-butyl-2-phenyl-1H-imidazol-5-yl)methyl]-N-(2,4- |
| | difluorobenzyl)methanamine |
| 482. | N-[(1-butyl-2-phenyl-1H-imidazol-5-yl)methyl]-2-phenyl-N-(4- |
| | propylbenzyl)ethanamine |
| 483. | N-benzyl-1-(1-butyl-2-phenyl-1H-imidazol-5-yl)-N-(4-propylbenzyl)methanamine |
| 484. | 1-(1-butyl-2-phenyl-1H-imidazol-5-yl)-N-(1-naphthylmethyl)-N-(4- |
| | propylbenzyl)methanamine |
| 485. | 1-(1,3-benzodioxol-5-yl)-N-[(1-butyl-2-phenyl-1H-imidazol-5-yl)methyl]-N-(4- |
| | propylbenzyl)methanamine |
| 486. | N-benzyl-1-(1-butyl-2-phenyl-1H-imidazol-5-yl)-N-(4-isopropylbenzyl)methanamine |
| 487. | 1-(1-butyl-2-phenyl-1H-imidazol-5-yl)-N-(4-isopropylbenzyl)-N-(1- |
| | naphthylmethyl)methanamine |
| 488. | 1-(1,3-benzodioxol-5-yl)-N-[(1-butyl-2-phenyl-1H-imidazol-5-yl)methyl]-N-(4- |
| | isopropylbenzyl)methanamine |
| 489. | N-[(1-butyl-2-phenyl-1H-imidazol-5-yl)methyl]-N-(3-ethoxybenzyl)-2- |
| | phenylethanamine |
| 490. | N-benzyl-1-(1-butyl-2-phenyl-1H-imidazol-5-yl)-N-(3-ethoxybenzyl)methanamine |
| 491. | 1-(1-butyl-2-phenyl-1H-imidazol-5-yl)-N-(3-ethoxybenzyl)-N-(1- |
| | naphthylmethyl)methanamine |
| 492. | 1-(1,3-benzodioxol-5-yl)-N-[(1-butyl-2-phenyl-1H-imidazol-5-yl)methyl]-N-(3- |
| | ethoxybenzyl)methanamine |
| 493. | 1-(1-butyl-2-phenyl-1H-imidazol-5-yl)-N-(4-ethoxybenzyl)-N-(1- |
| | naphthylmethyl)methanamine |
| 494. | 1-(1,3-benzodioxol-5-yl)-N-[(1-butyl-2-phenyl-1H-imidazol-5-yl)methyl]-N-(4- |
| | ethoxybenzyl)methanamine |
| 495. | N-[(1-butyl-2-phenyl-1H-imidazol-5-yl)methyl]-N-(2-ethoxybenzyl)-2- |
| | phenylethanamine |
| 496. | 1-(1,3-benzodioxol-5-yl)-N-[(1-butyl-2-phenyl-1H-imidazol-5-yl)methyl]-N-(2- |
| | ethoxybenzyl)methanamine |
| 497. | N-(1,3-benzodioxol-5-ylmethyl)-N-[(1-butyl-2-phenyl-1H-imidazol-5-yl)methyl]-2- |
| | phenylethanamine |
| 498. | 1-(1,3-benzodioxol-5-yl)-N-[(1-butyl-2-phenyl-1H-imidazol-5-yl)methyl]-N-(1- |
| | naphthylmethyl)methanamine |
| 499. | 1-(1,3-benzodioxol-5-yl)-N-(1,3-benzodioxol-5-ylmethyl)-N-[(1-butyl-2-phenyl-1H- |
| | imidazol-5-yl)methyl]methanamine |
| 500. | N-[(1-butyl-2-phenyl-1H-imidazol-5-yl)methyl]-N-[4-(methylthio)benzyl]-2- |
| | phenylethanamine |
| 501. | N-benzyl-1-(1-butyl-2-phenyl-1H-imidazol-5-yl)-N-[4- |
| | (methylthio)benzyl]methanamine |
| 502. | 1-(1-butyl-2-phenyl-1H-imidazol-5-yl)-N-[4-(methylthio)benzyl]-N-(1- |
| | naphthylmethyl)methanamine |
| 503. | 1-(1,3-benzodioxol-5-yl)-N-[(1-butyl-2-phenyl-1H-imidazol-5-yl)methyl]-N-[4- |
| | (methylthio)benzyl]methanamine |
| 504. | N-[(1-butyl-2-phenyl-1H-imidazol-5-yl)methyl]-N-(3-fluoro-4-methoxybenzyl)-2- |
| | phenylethanamine |
| 505. | N-benzyl-1-(1-butyl-2-phenyl-1H-imidazol-5-yl)-N-(3-fluoro-4- |
| | methoxybenzyl)methanamine |
| 506. | 1-(1-butyl-2-phenyl-1H-imidazol-5-yl)-N-(3-fluoro-4-methoxybenzyl)-N-(1- |
| | naphthylmethyl)methanamine |
| 507. | 1-(1,3-benzodioxol-5-yl)-N-[(1-butyl-2-phenyl-1H-imidazol-5-yl)methyl]-N-(3-fluoro- |
| | 4-methoxybenzyl)methanamine |
| 508. | N-benzyl-1-(1-butyl-2-phenyl-1H-imidazol-5-yl)-N-(4-chloro-3- |
| | methylbenzyl)methanamine |
| 509. | 1-(1-butyl-2-phenyl-1H-imidazol-5-yl)-N-(4-chloro-3-methylbenzyl)-N-(1- |
| | naphthylmethyl)methanamine |
| 510. | 1-(1,3-benzodioxol-5-yl)-N-[(1-butyl-2-phenyl-1H-imidazol-5-yl)methyl]-N-(4-chloro- |
| | 3-methylbenzyl)methanamine |
| 511. | 1-(1,3-benzodioxol-5-yl)-N-[(1-butyl-2-phenyl-1H-imidazol-5-yl)methyl]-N-(3-chloro- |
| | 4-fluorobenzyl)methanamine |
| 512. | N-benzyl-1-(4-butylphenyl)-N-[(1-butyl-2-phenyl-1H-imidazol-5- |
| | yl)methyl]methanamine |
| 513. | 1-(1,3-benzodioxol-5-yl)-N-(4-butylbenzyl)-N-[(1-butyl-2-phenyl-1H-imidazol-5- |
| | yl)methyl]methanamine |
| 514. | N-(4-tert-butylbenzyl)-N-[(1-butyl-2-phenyl-1H-imidazol-5-yl)methyl]-2- |
| | phenylethanamine |
| 515. | N-benzyl-1-(4-tert-butylphenyl)-N-[(1-butyl-2-phenyl-1H-imidazol-5- |
| | yl)methyl]methanamine |
| 516. | 1-(1,3-benzodioxol-5-yl)-N-(4-tert-butylbenzyl)-N-[(1-butyl-2-phenyl-1H-imidazol-5- |
| | yl)methyl]methanamine |
| 517. | 1-(1,3-benzodioxol-5-yl)-N-[(1-butyl-2-phenyl-1H-imidazol-5-yl)methyl]-N-(4- |
| | propoxybenzyl)methanamine |
| 518. | N-benzyl-1-(1-butyl-2-phenyl-1H-imidazol-5-yl)-N-(4-isopropoxybenzyl)methanamine |
| 519. | 1-(1-butyl-2-phenyl-1H-imidazol-5-yl)-N-(4-isopropoxybenzyl)-N-(1- |
| | naphthylmethyl)methanamine |
| 520. | 1-(1,3-benzodioxol-5-yl)-N-[(1-butyl-2-phenyl-1H-imidazol-5-yl)methyl]-N-(4- |
| | isopropoxybenzyl)methanamine |
| 521. | N-[(1-butyl-2-phenyl-1H-imidazol-5-yl)methyl]-N-[4-(ethylthio)benzyl]-2- |
| | phenylethanamine |
| 522. | N-benzyl-1-(1-butyl-2-phenyl-1H-imidazol-5-yl)-N-[4-(ethylthio)benzyl]methanamine |
| 523. | 1-(1-butyl-2-phenyl-1H-imidazol-5-yl)-N-[4-(ethylthio)benzyl]-N-(1- |
| | naphthylmethyl)methanamine |
| 524. | 1-(1,3-benzodioxol-5-yl)-N-[(1-butyl-2-phenyl-1H-imidazol-5-yl)methyl]-N-[4- |
| | (ethylthio)benzyl]methanamine |
| 525. | N-benzyl-1-(1-butyl-2-phenyl-1H-imidazol-5-yl)-N-(2,3,5,6- |
| | tetrafluorobenzyl)methanamine |
| 526. | 1-(1,3-benzodioxol-5-yl)-N-[(1-butyl-2-phenyl-1H-imidazol-5-yl)methyl]-N-(2,3,5,6- |
| | tetrafluorobenzyl)methanamine |
| 527. | 1-(1-butyl-2-phenyl-1H-imidazol-5-yl)-N-(1-naphthylmethyl)-N-(2,4,6- |
| | trifluorobenzyl)methanamine |
| 528. | 1-(1,3-benzodioxol-5-yl)-N-[(1-butyl-2-phenyl-1H-imidazol-5-yl)methyl]-N-(2,4,6- |
| | trifluorobenzyl)methanamine |
| 529. | 1-(1-butyl-2-phenyl-1H-imidazol-5-yl)-N-(1-naphthylmethyl)-N-(2,3,6- |
| | trifluorobenzyl)methanamine |
| 530. | 1-(1,3-benzodioxol-5-yl)-N-[(1-butyl-2-phenyl-1H-imidazol-5-yl)methyl]-N-(2,3,6- |
| | trifluorobenzyl)methanamine |
| 531. | N-benzyl-1-(1-butyl-2-phenyl-1H-imidazol-5-yl)-N-(2-chloro-6- |
| | fluorobenzyl)methanamine |
| 532. | 1-(1-butyl-2-phenyl-1H-imidazol-5-yl)-N-(2-chloro-6-fluorobenzyl)-N-(1- |
| | naphthylmethyl)methanamine |
| 533. | 1-(1,3-benzodioxol-5-yl)-N-[(1-butyl-2-phenyl-1H-imidazol-5-yl)methyl]-N-(2-chloro- |
| | 6-fluorobenzyl)methanamine |
| 534. | 1-(1-butyl-2-phenyl-1H-imidazol-5-yl)-N-(1-naphthylmethyl)-N-(2,4,6- |
| | trichlorobenzyl)methanamine |
| 535. | N-(1,3-benzodioxol-5-ylmethyl)-N-[(1-butyl-2-phenyl-1H-imidazol-5-yl)methyl]-2- |
| | (2,4-dimethoxyphenyl)ethanamine |
| 536. | N-benzyl-N-[(1-butyl-2-phenyl-1H-imidazol-5-yl)methyl]-2-(2,6- |
| | difluorophenyl)ethanamine |
| 537. | N-(1,3-benzodioxol-5-ylmethyl)-N-[(1-butyl-2-phenyl-1H-imidazol-5-yl)methyl]-2- |
| | (2,6-difluorophenyl)ethanamine |
| 538. | N-benzyl-N-[(1-butyl-2-phenyl-1H-imidazol-5-yl)methyl]-2-[4- |
| | (methylthio)phenyl]ethanamine |
| 539. | N-(1,3-benzodioxol-5-ylmethyl)-N-[(1-butyl-2-phenyl-1H-imidazol-5-yl)methyl]-2-[4- |
| | (methylthio)phenyl]ethanamine |
| 540. | 2-(1,3-benzodioxol-5-yl)-N-benzyl-N-[(1-butyl-2-phenyl-1H-imidazol-5- |
| | yl)methyl]ethanamine |
| 541. | 2-(1,3-benzodioxol-5-yl)-N-(1,3-benzodioxol-5-ylmethyl)-N-[(1-butyl-2-phenyl-1H- |
| | imidazol-5-yl)methyl]ethanamine |
| 542. | 1-(1,3-benzodioxol-5-yl)-N-[(1-butyl-2-phenyl-1H-imidazol-5-yl)methyl]-N-[2-chloro- |
| | 5-(methylthio)benzyl]methanamine |
| 543. | N-benzyl-1-(2-bromo-5-methylphenyl)-N-[(1-butyl-2-phenyl-1H-imidazol-5- |
| | yl)methyl]methanamine |
| 544. | 1-(1,3-benzodioxol-5-yl)-N-(2-bromo-5-methylbenzyl)-N-[(1-butyl-2-phenyl-1H- |
| | imidazol-5-yl)methyl]methanamine |
| 545. | 1-(1-butyl-2-phenyl-1H-imidazol-5-yl)-N-(2-chloro-4,5-dimethoxybenzyl)-N-(1- |
| | naphthylmethyl)methanamine |
| 546. | 1-(1,3-benzodioxol-5-yl)-N-(2-bromo-3-methylbenzyl)-N-[(1-butyl-2-phenyl-1H- |
| | imidazol-5-yl)methyl]methanamine |
| 547. | 1-(1,3-benzodioxol-5-yl)-N-[(1-butyl-2-phenyl-1H-imidazol-5-yl)methyl]-N-[2-chloro- |
| | 5-(trifluoromethyl)benzyl]methanamine |
| 548. | 1-(2-bromo-4,5-dimethoxyphenyl)-N-[(1-butyl-2-phenyl-1H-imidazol-5-yl)methyl]-N- |
| | (1-naphthylmethyl)methanamine |
| 549. | N-benzyl-1-(1-butyl-2-phenyl-1H-imidazol-5-yl)-N-[2- |
| | (methylthio)benzyl]methanamine |
| 550. | 1-(1,3-benzodioxol-5-yl)-N-[(1-butyl-2-phenyl-1H-imidazol-5-yl)methyl]-N-[2- |
| | (methylthio)benzyl]methanamine |
| 551. | N-(1,3-benzodioxol-5-ylmethyl)-2-(2-bromo-4,5-dimethoxyphenyl)-N-[(1-butyl-2- |
| | phenyl-1H-imidazol-5-yl)methyl]ethanamine |
| 552. | N-(1,3-benzodioxol-5-ylmethyl)-2-(5-bromo-2-methoxyphenyl)-N-[(1-butyl-2-phenyl- |
| | 1H-imidazol-5-yl)methyl]ethanamine |
| 553. | N-(1,3-benzodioxol-5-ylmethyl)-2-[4-(benzyloxy)phenyl]-N-[(1-butyl-2-phenyl-1H- |
| | imidazol-5-yl)methyl]ethanamine |
| 554. | N-(1,3-benzodioxol-5-ylmethyl)-N-[(1-butyl-2-phenyl-1H-imidazol-5-yl)methyl]-2- |
| | (2,3-dimethoxyphenyl)ethanamine |
| 555. | N-(1,3-benzodioxol-5-ylmethyl)-N-[(1-butyl-2-phenyl-1H-imidazol-5-yl)methyl]-2- |
| | (2,3-difluorophenyl)ethanamine |
| 556. | N-(1,3-benzodioxol-5-ylmethyl)-N-[(1-butyl-2-phenyl-1H-imidazol-5-yl)methyl]-2-(2- |
| | phenoxyphenyl)ethanamine |
| 557. | 1-(1,3-benzodioxol-5-yl)-N-[(1-butyl-2-phenyl-1H-imidazol-5-yl)methyl]-N-(3-iodo-4- |
| | methylbenzyl)methanamine |
| 558. | N-(1,3-benzodioxol-5-ylmethyl)-N-[(1-butyl-2-phenyl-1H-imidazol-5-yl)methyl]-2-(4- |
| | iodophenyl)ethanamine |
| 559. | N-(1,3-benzodioxol-5-ylmethyl)-N-{[1-butyl-2-phenyl-1H-imidazol-5-yl]methyl}-2- |
| | (phenyl)ethanamine |
| 560. | 1-(1,3-benzodioxol-5-yl)-N-[(1-butyl-2-phenyl-1H-imidazol-5-yl)methyl]-N-[(5- |
| | methylthien-2-yl)methyl]methanamine |
| 561. | N-(1,3-benzodioxol-5-ylmethyl)-N-[(1-butyl-2-phenyl-1H-imidazol-5-yl)methyl]-2-(4- |
| | methylphenyl)ethanamine |
| 562. | N-(1,3-benzodioxol-5-ylmethyl)-N-[(1-butyl-2-phenyl-1H-imidazol-5-yl)methyl]-2-(2- |
| | methylphenyl)ethanamine |
| 563. | N-benzyl-N-[(1-butyl-2-phenyl-1H-imidazol-5-yl)methyl]-2-phenylpropan-1-amine |
| 564. | N-(1,3-benzodioxol-5-ylmethyl)-N-[(1-butyl-2-phenyl-1H-imidazol-5-yl)methyl]-2- |
| | phenylpropan-1-amine |
| 565. | N-(1,3-benzodioxol-5-ylmethyl)-N-[(1-butyl-2-phenyl-1H-imidazol-5-yl)methyl]-2- |
| | phenoxyethanamine |
| 566. | N-(1,3-benzodioxol-5-ylmethyl)-N-[(1-butyl-2-phenyl-1H-imidazol-5-yl)methyl]-2-(2- |
| | fluorophenyl)ethanamine |
| 567. | N-benzyl-N-[(1-butyl-2-phenyl-1H-imidazol-5-yl)methyl]-2-phenylbutan-1-amine |
| 568. | N-(1,3-benzodioxol-5-ylmethyl)-N-[(1-butyl-2-phenyl-1H-imidazol-5-yl)methyl]-2- |
| | phenylbutan-1-amine |
| 569. | N-benzyl-N-[(1-butyl-2-phenyl-1H-imidazol-5-yl)methyl]-2-(2-methylphenyl)propan- |
| | 1-amine |
| 570. | N-(1,3-benzodioxol-5-ylmethyl)-N-[(1-butyl-2-phenyl-1H-imidazol-5-yl)methyl]-2-(2- |
| | methylphenyl)propan-1-amine |
| 571. | N-(1,3-benzodioxol-5-ylmethyl)-N-[(1-butyl-2-phenyl-1H-imidazol-5-yl)methyl]-2-(3- |
| | methoxyphenyl)ethanamine |
| 572. | N-(1,3-benzodioxol-5-ylmethyl)-N-[(1-butyl-2-phenyl-1H-imidazol-5-yl)methyl]-2-(4- |
| | methoxyphenyl)ethanamine |
| 573. | (2Z)-N-(1,3-benzodioxol-5-ylmethyl)-N-[(1-butyl-2-phenyl-1H-imidazol-5-yl)methyl]- |
| | 3-(2-fluorophenyl)prop-2-en-1-amine |
| 574. | N-(1,3-benzodioxol-5-ylmethyl)-N-[(1-butyl-2-phenyl-1H-imidazol-5-yl)methyl]-2-(4- |
| | ethoxyphenyl)ethanamine |
| 575. | N-(1,3-benzodioxol-5-ylmethyl)-N-[(1-butyl-2-phenyl-1H-imidazol-5-yl)methyl]-2-(2- |
| | ethoxyphenyl)ethanamine |
| 576. | N-(1,3-benzodioxol-5-ylmethyl)-N-[(1-butyl-2-phenyl-1H-imidazol-5-yl)methyl]-2-(3- |
| | methylphenoxy)ethanamine |
| 577. | N-(1,3-benzodioxol-5-ylmethyl)-N-[(1-butyl-2-phenyl-1H-imidazol-5-yl)methyl]-2-(3- |
| | methoxyphenoxy)ethanamine |
| 578. | (2Z)-N-[(1-butyl-2-phenyl-1H-imidazol-5-yl)methyl]-3-(2,5-dimethoxyphenyl)-N-(1- |
| | naphthylmethyl)prop-2-en-1-amine |
| 579. | (2Z)-N-(1,3-benzodioxol-5-ylmethyl)-N-[(1-butyl-2-phenyl-1H-imidazol-5-yl)methyl]- |
| | 3-(2,5-dimethoxyphenyl)prop-2-en-1-amine |
| 580. | (2Z)-N-[(1-butyl-2-phenyl-1H-imidazol-5-yl)methyl]-3-(3,5-dimethoxyphenyl)-N-(2- |
| | phenylethyl)prop-2-en-1-amine |
| 581. | (2Z)-N-[(1-butyl-2-phenyl-1H-imidazol-5-yl)methyl]-3-(3,5-dimethoxyphenyl)-N-(1- |
| | naphthylmethyl)prop-2-en-1-amine |
| 582. | (2Z)-N-(1,3-benzodioxol-5-ylmethyl)-N-[(1-butyl-2-phenyl-1H-imidazol-5-yl)methyl]- |
| | 3-(3,5-dimethoxyphenyl)prop-2-en-1-amine |
| 583. | (2Z)-N-(1,3-benzodioxol-5-ylmethyl)-N-[(1-butyl-2-phenyl-1H-imidazol-5-yl)methyl]- |
| | 3-[3-(trifluoromethyl)phenyl]prop-2-en-1-amine |
| 584. | (2Z)-N-(1,3-benzodioxol-5-ylmethyl)-N-[(1-butyl-2-phenyl-1H-imidazol-5-yl)methyl]- |
| | 3-(2,6-dichlorophenyl)prop-2-en-1-amine |
| 585. | (2Z)-3-(3-bromophenyl)-N-[(1-butyl-2-phenyl-1H-imidazol-5-yl)methyl]-N-(2- |
| | phenylethyl)prop-2-en-1-amine |
| 586. | (2Z)-N-(1,3-benzodioxol-5-ylmethyl)-3-(3-bromophenyl)-N-[(1-butyl-2-phenyl-1H- |
| | imidazol-5-yl)methyl]prop-2-en-1-amine |
| 587. | (2Z)-N-benzyl-N-[(1-butyl-2-phenyl-1H-imidazol-5-yl)methyl]-3-thien-3-ylprop-2-en- |
| | 1-amine |
| 588. | (2Z)-N-[(1-butyl-2-phenyl-1H-imidazol-5-yl)methyl]-N-(1-naphthylmethyl)-3-thien-3- |
| | ylprop-2-en-1-amine |
| 589. | (2Z)-N-(1,3-benzodioxol-5-ylmethyl)-N-[(1-butyl-2-phenyl-1H-imidazol-5-yl)methyl]- |
| | 3-thien-3-ylprop-2-en-1-amine |
| 590. | (2Z)-N-[(1-butyl-2-phenyl-1H-imidazol-5-yl)methyl]-3-(2-furyl)-N-(1- |
| | naphthylmethyl)prop-2-en-1-amine |
| 591. | (2Z)-N-(1,3-benzodioxol-5-ylmethyl)-N-[(1-butyl-2-phenyl-1H-imidazol-5-yl)methyl]- |
| | 3-(2-furyl)prop-2-en-1-amine |
| 592. | (2Z)-N-(1,3-benzodioxol-5-ylmethyl)-N-[(1-butyl-2-phenyl-1H-imidazol-5-yl)methyl]- |
| | 3-(4-isopropylphenyl)prop-2-en-1-amine |
| 593. | N-[(1-butyl-2-phenyl-1H-imidazol-5-yl)methyl]-2-phenyl-N-[(2- |
| | phenylcyclopropyl)methyl]ethanamine |
| 594. | N-benzyl-1-(1-butyl-2-phenyl-1H-imidazol-5-yl)-N-[(2- |
| | phenylcyclopropyl)methyl]methanamine |
| 595. | 1-(1,3-benzodioxol-5-yl)-N-[(1-butyl-2-phenyl-1H-imidazol-5-yl)methyl]-N-[(2- |
| | phenylcyclopropyl)methyl]methanamine |
| 596. | (2E)-N-benzyl-N-[(1-butyl-2-phenyl-1H-imidazol-5-yl)methyl]-2-fluoro-3-phenylprop- |
| | 2-en-1-amine |
| 597. | (2E)-N-[(1-butyl-2-phenyl-1H-imidazol-5-yl)methyl]-2-fluoro-N-(1-naphthylmethyl)-3- |
| | phenylprop-2-en-1-amine |
| 598. | (2E)-N-(1,3-benzodioxol-5-ylmethyl)-N-[(1-butyl-2-phenyl-1H-imidazol-5-yl)methyl]- |
| | 2-fluoro-3-phenylprop-2-en-1-amine |
| 599. | N-benzyl-N-[(1-butyl-2-phenyl-1H-imidazol-5-yl)methyl]-2-(2- |
| | chlorophenyl)ethanamine |
| 600. | N-(1,3-benzodioxol-5-ylmethyl)-N-[(1-butyl-2-phenyl-1H-imidazol-5-yl)methyl]-2-(2- |
| | chlorophenyl)ethanamine |
| 601. | N-benzyl-N-[(1-butyl-2-phenyl-1H-imidazol-5-yl)methyl]-3-methyl-2-phenylbutan-1- |
| | amine |
| 602. | N-(1,3-benzodioxol-5-ylmethyl)-N-[(1-butyl-2-phenyl-1H-imidazol-5-yl)methyl]-3- |
| | methyl-2-phenylbutan-1-amine |
| 603. | N-benzyl-N-[(1-butyl-2-phenyl-1H-imidazol-5-yl)methyl]-2-(4- |
| | ethoxyphenyl)ethanamine |
| 604. | N-(1,3-benzodioxol-5-ylmethyl)-N-{[1-butyl-2-phenyl-1H-imidazol-5-yl]methyl}-2-(4- |
| | ethoxyphenyl)ethanamine |
| 605. | N-benzyl-N-[(1-butyl-2-phenyl-1H-imidazol-5-yl)methyl]-2-(2- |
| | ethoxyphenyl)ethanamine |
| 606. | N-(1,3-benzodioxol-5-ylmethyl)-N-{[1-butyl-2-phenyl-1H-imidazol-5-yl]methyl}-2-(2- |
| | ethoxyphenyl)ethanamine |
| 607. | N-benzyl-N-[(1-butyl-2-phenyl-1H-imidazol-5-yl)methyl]-2-(4-methoxyphenyl)butan- |
| | 1-amine |
| 608. | N-(1,3-benzodioxol-5-ylmethyl)-N-[(1-butyl-2-phenyl-1H-imidazol-5-yl)methyl]-2-(4- |
| | methoxyphenyl)butan-1-amine |
| 609. | N-benzyl-N-[(1-butyl-2-phenyl-1H-imidazol-5-yl)methyl]-2-(2,5- |
| | dimethoxyphenyl)ethanamine |
| 610. | N-(1,3-benzodioxol-5-ylmethyl)-N-[(1-butyl-2-phenyl-1H-imidazol-5-yl)methyl]-2- |
| | (2,5-dimethoxyphenyl)ethanamine |
| 611. | N-(1,3-benzodioxol-5-ylmethyl)-N-[(1-butyl-2-phenyl-1H-imidazol-5-yl)methyl]-2-[4- |
| | (trifluoromethyl)phenyl]ethanamine |
| 612. | N-(1,3-benzodioxol-5-ylmethyl)-N-[(1-butyl-2-phenyl-1H-imidazol-5-yl)methyl]-2- |
| | cyclopentyl-2-phenylethanamine |
| 613. | N-(1,3-benzodioxol-5-ylmethyl)-N-[(1-butyl-2-phenyl-1H-imidazol-5-yl)methyl]-2- |
| | (2,4-dichlorophenyl)ethanamine |
| 614. | N-(1,3-benzodioxol-5-ylmethyl)-N-[(1-butyl-2-phenyl-1H-imidazol-5-yl)methyl]-2,2- |
| | diphenylethanamine |
| 615. | N-(1,3-benzodioxol-5-ylmethyl)-2-(3-bromophenyl)-N-[(1-butyl-2-phenyl-1H- |
| | imidazol-5-yl)methyl]ethanamine |
| 616. | N-(1,3-benzodioxol-5-ylmethyl)-2-(4-bromophenyl)-N-[(1-butyl-2-phenyl-1H- |
| | imidazol-5-yl)methyl]ethanamine |
| 617. | N-(1,3-benzodioxol-5-ylmethyl)-2-(2-bromophenyl)-N-[(1-butyl-2-phenyl-1H- |
| | imidazol-5-yl)methyl]ethanamine |
| 618. | N-(1,3-benzodioxol-5-ylmethyl)-N-[(1-butyl-2-phenyl-1H-imidazol-5-yl)methyl]-2- |
| | cyclohexyl-2-phenylethanamine |
| 619. | 1-(1,3-benzodioxol-5-yl)-N-[(1-butyl-2-phenyl-1H-imidazol-5-yl)methyl]-N-(2,3- |
| | dimethoxybenzyl)methanamine |
| 620. | 1-(1,3-benzodioxol-5-yl)-N-[(1-butyl-2-phenyl-1H-imidazol-5-yl)methyl]-N-(2,5- |
| | dimethoxybenzyl)methanamine |
| 621. | 1-(1,3-benzodioxol-5-yl)-N-[(1-butyl-2-phenyl-1H-imidazol-5-yl)methyl]-N-(4-chloro- |
| | 2-methoxybenzyl)methanamine |
| 622. | N-benzyl-1-(3-bromo-4-methylphenyl)-N-[(1-butyl-2-phenyl-1H-imidazol-5- |
| | yl)methyl]methanamine |
| 623. | N-(3-bromo-4-methylbenzyl)-N-[(1-butyl-2-phenyl-1H-imidazol-5-yl)methyl]-2- |
| | phenylethanamine |
| 624. | 1-(3-bromo-4-methylphenyl)-N-[(1-butyl-2-phenyl-1H-imidazol-5-yl)methyl]-N-( 1- |
| | naphthylmethyl)methanamine |
| 625. | 1-(1,3-benzodioxol-5-yl)-N-(3-bromo-4-methylbenzyl)-N-[(1-butyl-2-phenyl-1H- |
| | imidazol-5-yl)methyl]methanamine |
| 626. | 1-(1,3-benzodioxol-5-yl)-N-(3-bromo-4-fluorobenzyl)-N-[(1-butyl-2-phenyl-1H- |
| | imidazol-5-yl)methyl]methanamine |
| 627. | 1-(1,3-benzodioxol-5-yl)-N-[(1-butyl-2-phenyl-1H-imidazol-5-yl)methyl]-N-[2-(2- |
| | phenylethyl)benzyl]methanamine |
| 628. | N-benzyl-1-(1-butyl-2-phenyl-1H-imidazol-5-yl)-N-(3-iodobenzyl)methanamine |
| 629. | 1-(1,3-benzodioxol-5-yl)-N-[(1-butyl-2-phenyl-1H-imidazol-5-yl)methyl]-N-(3- |
| | iodobenzyl)methanamine |
| 630. | N-benzyl-1-(1-butyl-2-phenyl-1H-imidazol-5-yl)-N-(4-iodobenzyl)methanamine |
| 631. | 1-(1,3-benzodioxol-5-yl)-N-[(1-butyl-2-phenyl-1H-imidazol-5-yl)methyl]-N-(4- |
| | iodobenzyl)methanamine |
| 632. | 1-(1,3-benzodioxol-5-yl)-N-[(1-butyl-2-phenyl-1H-imidazol-5-yl)methyl]-N-(2- |
| | iodobenzyl)methanamine |
| 633. | (2Z)-N-(1,3-benzodioxol-5-ylmethyl)-3-(5-bromo-2-fluorophenyl)-N-[(1-butyl-2- |
| | phenyl-1H-imidazol-5-yl)methyl]prop-2-en-1-amine |
| 634. | (2Z)-N-(1,3-benzodioxol-5-ylmethyl)-3-(4-bromo-2-fluorophenyl)-N-[(1-butyl-2- |
| | phenyl-1H-imidazol-5-yl)methyl]prop-2-en-1-amine |
| 635. | (2Z)-N-(1,3-benzodioxol-5-ylmethyl)-N-[(1-butyl-2-phenyl-1H-imidazol-5-yl)methyl]- |
| | 3-(4-chloro-2-fluorophenyl)prop-2-en-1-amine |
| 636. | (2Z)-N-(1,3-benzodioxol-5-ylmethyl)-N-[(1-butyl-2-phenyl-1H-imidazol-5-yl)methyl]- |
| | 3-(2-chloro-6-fluorophenyl)prop-2-en-1-amine |
| 637. | (2Z)-N-(1,3-benzodioxol-5-ylmethyl)-3-(5-bromo-2-ethoxyphenyl)-N-[(1-butyl-2- |
| | phenyl-1H-imidazol-5-yl)methyl]prop-2-en-1-amine |
| 638. | N-(1,3-benzodioxol-5-ylmethyl)-N-{[1-butyl-2-(4-fluorophenyl)-1H-imidazol-5- |
| | yl]methyl}butan-1-amine |
| 639. | N-(1,3-benzodioxol-5-ylmethyl)-N-{[1-butyl-2-(4-fluorophenyl)-1H-imidazol-5- |
| | yl]methyl}-2-methylpropan-1-amine |
| 640. | 1-(1,3-benzodioxol-5-yl)-N-{[1-butyl-2-(4-fluorophenyl)-1H-imidazol-5-yl]methyl}-N- |
| | (cyclobutylmethyl)methanamine |
| 641. | N-(1,3-benzodioxol-5-ylmethyl)-N-{[1-butyl-2-(4-fluorophenyl)-1H-imidazol-5- |
| | yl]methyl}pentan-1-amine |
| 642. | N-(1,3-benzodioxol-5-ylmethyl)-N-{[1-butyl-2-(4-fluorophenyl)-1H-imidazol-5- |
| | yl]methyl}-3-methylbutan-1-amine |
| 643. | N-(1,3-benzodioxol-5-ylmethyl)-N-{[1-butyl-2-(4-fluorophenyl)-1H-imidazol-5- |
| | yl]methyl}-2,2-dimethylpropan-1-amine |
| 644. | 1-(1,3-benzodioxol-5-yl)-N-{[1-butyl-2-(4-fluorophenyl)-1H-imidazol-5-yl]methyl}-N- |
| | (cyclopentylmethyl)methanamine |
| 645. | N-(1,3-benzodioxol-5-ylmethyl)-N-{[1-butyl-2-(4-fluorophenyl)-1H-imidazol-5- |
| | yl]methyl}hexan-1-amine |
| 646. | N-(1,3-benzodioxol-5-ylmethyl)-N-{[1-butyl-2-(4-fluorophenyl)-1H-imidazol-5- |
| | yl]methyl}-4-methylpentan-1-amine |
| 647. | N-(1,3-benzodioxol-5-ylmethyl)-N-{[1-butyl-2-(4-fluorophenyl)-1H-imidazol-5- |
| | yl]methyl}-3,3-dimethylbutan-1-amine |
| 648. | N-(1,3-benzodioxol-5-ylmethyl)-N-{[1-butyl-2-(4-fluorophenyl)-1H-imidazol-5- |
| | yl]methyl}-2-ethylbutan-1-amine |
| 649. | N-(1,3-benzodioxol-5-ylmethyl)-N-{[1-butyl-2-(4-fluorophenyl)-1H-imidazol-5- |
| | yl]methyl}-2,2-dimethylbutan-1-amine |
| 650. | N-(1,3-benzodioxol-5-ylmethyl)-N-{[1-butyl-2-(4-fluorophenyl)-1H-imidazol-5- |
| | yl]methyl}-2-cyclopentylethanamine |
| 651. | 1-(1,3-benzodioxol-5-yl)-N-{[1-butyl-2-(4-fluorophenyl)-1H-imidazol-5-yl]methyl}-N- |
| | (cyclohexylmethyl)methanamine |
| 652. | N-(1,3-benzodioxol-5-ylmethyl)-N-{[1-butyl-2-(4-fluorophenyl)-1H-imidazol-5- |
| | yl]methyl}heptan-1-amine |
| 653. | N-(1,3-benzodioxol-5-ylmethyl)-N-{[1-butyl-2-(4-fluorophenyl)-1H-imidazol-5- |
| | yl]methyl}-2-cyclohexylethanamine |
| 654. | 1-(1,3-benzodioxol-5-yl)-N-{[1-butyl-2-(4-fluorophenyl)-1H-imidazol-5-yl]methyl}-N- |
| | (cycloheptylmethyl)methanamine |
| 655. | N-(1,3-benzodioxol-5-ylmethyl)-N-{[1-butyl-2-(4-fluorophenyl)-1H-imidazol-5- |
| | yl]methyl}-3-cyclopentylpropan-1-amine |
| 656. | N-(1,3-benzodioxol-5-ylmethyl)-N-{[1-butyl-2-(4-fluorophenyl)-1H-imidazol-5- |
| | yl]methyl}octan-1-amine |
| 657. | N-(1,3-benzodioxol-5-ylmethyl)-N-{[1-butyl-2-(3-fluorophenyl)-1H-imidazol-5- |
| | yl]methyl}-2-ethylbutan-1-amine |
| 658. | N-(1,3-benzodioxol-5-ylmethyl)-N-{[1-butyl-2-(3-fluorophenyl)-1H-imidazol-5- |
| | yl]methyl}-2,2-dimethylbutan-1-amine |
| 659. | N-(1,3-benzodioxol-5-ylmethyl)-N-{[1-butyl-2-(3-fluorophenyl)-1H-imidazol-5- |
| | yl]methyl}-2-cyclopentylethanamine |
| 660. | 1-(1,3-benzodioxol-5-yl)-N-{[1-butyl-2-(3-fluorophenyl)-1H-imidazol-5-yl]methyl}-N- |
| | (cyclohexylmethyl)methanamine |
| 661. | N-(1,3-benzodioxol-5-ylmethyl)-N-{[1-butyl-2-(3-fluorophenyl)-1H-imidazol-5- |
| | yl]methyl}heptan-1-amine |
| 662. | 1-(1,3-benzodioxol-5-yl)-N-benzyl-N-{[1-butyl-2-(4-fluorophenyl)-1H-imidazol-5- |
| | yl]methyl}methanamine |
| 663. | 1-(1,3-benzodioxol-5-yl)-N-{[1-butyl-2-(4-fluorophenyl)-1H-imidazol-5-yl]methyl}-N- |
| | (3-methylbenzyl)methanamine |
| 664. | 1-(1,3-benzodioxol-5-yl)-N-{[1-butyl-2-(4-fluorophenyl)-1H-imidazol-5-yl]methyl}-N- |
| | (4-methylbenzyl)methanamine |
| 665. | N-{[1-butyl-2-(4-fluorophenyl)-1H-imidazol-5-yl]methyl}-N-(cyclohexylmethyl)-N-(4- |
| | methylbenzyl)amine |
| 666. | 1-(1,3-benzodioxol-5-yl)-N-{[1-butyl-2-(4-fluorophenyl)-1H-imidazol-5-yl]methyl}-N- |
| | (2-fluorobenzyl)methanamine |
| 667. | N-{[1-butyl-2-(4-fluorophenyl)-1H-imidazol-5-yl]methyl}-N-(3,4-dimethylbenzyl)-3- |
| | methylbutan-1-amine |
| 668. | 1-(1,3-benzodioxol-5-yl)-N-{[1-butyl-2-(4-fluorophenyl)-1H-imidazol-5-yl]methyl}-N- |
| | (3,4-dimethylbenzyl)methanamine |
| 669. | N-{[1-butyl-2-(4-fluorophenyl)-1H-imidazol-5-yl]methyl}-N-(cyclohexylmethyl)-N- |
| | (3,4-dimethylbenzyl)amine |
| 670. | 1-(1,3-benzodioxol-5-yl)-N-{[1-butyl-2-(4-fluorophenyl)-1H-imidazol-5-yl]methyl}-N- |
| | (2,3-dimethylbenzyl)methanamine |
| 671. | N-(2,4-dimethylphenylmethyl)-N-{[1-butyl-2-(4-fluorophenyl)-1H-imidazol-5- |
| | yl]methyl}-4-methylpentan-1-amine |
| 672. | 1-(1,3-benzodioxol-5-yl)-N-(1,3-benzodioxol-5-ylmethyl)-N-{[1-butyl-2-(4- |
| | fluorophenyl)-1H-imidazol-5-yl]methyl}methanamine |
| 673. | N-(1,3-benzodioxol-5-ylmethyl)-N-{[1-butyl-2-(4-fluorophenyl)-1H-imidazol-5- |
| | yl]methyl}-2-(cyclohexylmethyl)ethanamine |
| 674. | 1-(1,3-benzodioxol-5-yl)-N-{[1-butyl-2-(4-fluorophenyl)-1H-imidazol-5-yl]methyl}-N- |
| | [4-(methylthio)benzyl]methanamine |
| 675. | N-benzyl-1-[1-butyl-2-(3-fluorophenyl)-1H-imidazol-5-yl]-N- |
| | (cyclohexylmethyl)methanamine |
| 676. | N-{[1-butyl-2-(3-fluorophenyl)-1H-imidazol-5-yl]methyl}-3-methyl-N-(4- |
| | methylbenzyl)butan-1-amine |
| 677. | N-{[1-butyl-2-(3-fluorophenyl)-1H-imidazol-5-yl]methyl}-N-(cyclohexylmethyl)-N-(4- |
| | methylbenzyl)amine |
| 678. | N-{[1-butyl-2-(3-fluorophenyl)-1H-imidazol-5-yl]methyl}-N-(3,4-dimethylbenzyl)-3- |
| | methylbutan-1-amine |
| 679. | N-{[1-butyl-2-(3-fluorophenyl)-1H-imidazol-5-yl]methyl}-N-(cyclohexylmethyl)-N-(4- |
| | methoxybenzyl)amine |
| 680. | N-{[1-butyl-2-(3-fluorophenyl)-1H-imidazol-5-yl]methyl}-3-methyl-N-(4- |
| | propylbenzyl)butan-1-amine |
| 681. | N-{[1-butyl-2-(3-fluorophenyl)-1H-imidazol-5-yl]methyl}-N-(4-isopropylbenzyl)-3- |
| | methylbutan-1-amine |
| 682. | N-{[1-butyl-2-(3-fluorophenyl)-1H-imidazol-5-yl]methyl}-N-(cyclohexylmethyl)-N-(4- |
| | isopropylbenzyl)amine |
| 683. | N-(1,3-benzodioxol-5-ylmethyl)-N-{[1-butyl-2-(3-fluorophenyl)-1H-imidazol-5- |
| | yl]methyl}-3-methylbutan-1-amine |
| 684. | 1-(1,3-benzodioxol-5-yl)-N-(1,3-benzodioxol-5-ylmethyl)-N-{[1-butyl-2-(3- |
| | fluorophenyl)-1H-imidazol-5-yl]methyl}methanamine |
| 685. | N-(1,3-benzodioxol-5-ylmethyl)-N-{[1-butyl-2-(3-fluorophenyl)-1H-imidazol-5- |
| | yl]methyl}-2-(cyclohexylmethyl)ethanamine |
| 686. | N-{[1-butyl-2-(3-fluorophenyl)-1H-imidazol-5-yl]methyl}-3-methyl-N-[4- |
| | (methylthio)benzyl]butan-1-amine |
| 687. | 1-(1,3-benzodioxol-5-yl)-N-{[1-butyl-2-(3-fluorophenyl)-1H-imidazol-5-yl]methyl}-N- |
| | [4-(methylthio)benzyl]methanamine |
| 688. | N-(4-butylbenzyl)-N-{[1-butyl-2-(3-fluorophenyl)-1H-imidazol-5-yl]methyl}-3- |
| | methylbutan-1-amine |
| 689. | N-{[1-butyl-2-(3-fluorophenyl)-1H-imidazol-5-yl]methyl}-N-[4-(ethylthio)benzyl]-3- |
| | methylbutan-1-amine |
| 690. | 1-(1,3-benzodioxol-5-yl)-N-{[1-butyl-2-(4-fluorophenyl)-1H-imidazol-5-yl]methyl}-N- |
| | (3,5-dimethoxybenzyl)methanamine |
| 691. | 1-(1,3-benzodioxol-5-yl)-N-{[1-butyl-2-(4-fluorophenyl)-1H-imidazol-5-yl]methyl}-N- |
| | (2,5-dimethoxybenzyl)methanamine |
| 692. | 1-(1,3-benzodioxol-5-yl)-N-{[1-butyl-2-(4-fluorophenyl)-1H-imidazol-5-yl]methyl}-N- |
| | (2,4-dimethoxybenzyl)methanamine |
| 693. | 1-(1,3-benzodioxol-5-yl)-N-{[1-butyl-2-(4-fluorophenyl)-1H-imidazol-5-yl]methyl}-N- |
| | (2,5-dichlorobenzyl)methanamine |
| 694. | 1-(1,3-benzodioxol-5-yl)-N-(3-bromobenzyl)-N-{[1-butyl-2-(4-fluorophenyl)-1H- |
| | imidazol-5-yl]methyl}methanamine |
| 695. | 1-(1,3-benzodioxol-5-yl)-N-(4-bromobenzyl)-N-{[1-butyl-2-(4-fluorophenyl)-1H- |
| | imidazol-5-yl]methyl}methanamine |
| 696. | 1-(1,3-benzodioxol-5-yl)-N-(2-bromobenzyl)-N-{[1-butyl-2-(4-fluorophenyl)-1H- |
| | imidazol-5-yl]methyl}methanamine |
| 697. | 1-(1,3-benzodioxol-5-yl)-N-{[1-butyl-2-(4-fluorophenyl)-1H-imidazol-5-yl]methyl}-N- |
| | [2-(2-phenylethyl)benzyl]methanamine |
| 698. | N-(2-fluorobenzyl)-3-methyl-N-{[2-phenyl-1-(2-phenylethyl)-1H-imidazol-5- |
| | yl]methyl}butan-1-amine |
| 699. | 1-(1,3-benzodioxol-5-yl)-N-(2-fluorobenzyl)-N-{[2-phenyl-1-(2-phenylethyl)-1H- |
| | imidazol-5-yl]methyl}methanamine |
| 700. | N-(4-ethylbenzyl)-3-methyl-N-{[2-phenyl-1-(2-phenylethyl)-1H-imidazol-5- |
| | yl]methyl}butan-1-amine |
| 701. | N-(3,4-dimethylbenzyl)-3-methyl-N-{[2-phenyl-1-(2-phenylethyl)-1H-imidazol-5- |
| | yl]methyl}butan-1-amine |
| 702. | N-(3,5-dimethylbenzyl)-3-methyl-N-{[2-phenyl-1-(2-phenylethyl)-1H-imidazol-5- |
| | yl]methyl}butan-1-amine |
| 703. | N-(2,3-dimethylbenzyl)-3-methyl-N-{[2-phenyl-1-(2-phenylethyl)-1H-imidazol-5- |
| | yl]methyl}butan-1-amine |
| 704. | 1-(1,3-benzodioxol-5-yl)-N-{[1-butyl-2-(4-fluorophenyl)-1H-imidazol-5-yl]methyl}-N- |
| | (2,4,6-trifluorobenzyl)methanamine |
| 705. | 1-(1,3-benzodioxol-5-yl)-N-{[1-butyl-2-(4-fluorophenyl)-1H-imidazol-5-yl]methyl}-N- |
| | (2,3,6-trifluorobenzyl)methanamine |
| 706. | N-(1,3-benzodioxol-5-ylmethyl)-N-{[1-butyl-2-(4-fluorophenyl)-1H-imidazol-5- |
| | yl]methyl}-2-(3,4-dimethoxyphenyl)ethanamine |
| 707. | 2-(1,3-benzodioxol-5-yl)-N-(1,3-benzodioxol-5-ylmethyl)-N-{[1-butyl-2-(4- |
| | fluorophenyl)-1H-imidazol-5-yl]methyl}ethanamine |
| 708. | N-(1,3-benzodioxol-5-ylmethyl)-N-{[1-butyl-2-(4-fluorophenyl)-1H-imidazol-5- |
| | yl]methyl}-2-(2-naphthyl)ethanamine |
| 709. | N-(1,3-benzodioxol-5-ylmethyl)-N-{[1-butyl-2-(4-fluorophenyl)-1H-imidazol-5- |
| | yl]methyl}-2-(3-methyl-1-benzothien-2-yl)ethanamine |
| 710. | 1-(1,3-benzodioxol-5-yl)-N-{[1-butyl-2-(3-fluorophenyl)-1H-imidazol-5-yl]methyl}-N- |
| | (2,5-dimethoxybenzyl)methanamine |
| 711. | 1-(1,3-benzodioxol-5-yl)-N-{[1-butyl-2-(3-fluorophenyl)-1H-imidazol-5-yl]methyl}-N- |
| | (2,4-dimethoxybenzyl)methanamine |
| 712. | N-{[1-butyl-2-(3-fluorophenyl)-1H-imidazol-5-yl]methyl}-N-(3-chloro-4- |
| | methoxybenzyl)-3-methylbutan-1-amine |
| 713. | N-{[1-butyl-2-(3-fluorophenyl)-1H-imidazol-5-yl]methyl}-N-(cyclohexylmethyl)-N- |
| | (2,3,4-trimethoxybenzyl)amine |
| 714. | N-(1,3-benzodioxol-5-ylmethyl)-N-{[1-butyl-2-(4-fluorophenyl)-1H-imidazol-5- |
| | yl]methyl}-2-(4-methylphenyl)ethanamine |
| 715. | N-(1,3-benzodioxol-5-ylmethyl)-N-{[1-butyl-2-(4-fluorophenyl)-1H-imidazol-5- |
| | yl]methyl}-2-(3-methoxyphenyl)ethanamine |
| 716. | N-(1,3-benzodioxol-5-ylmethyl)-N-{[1-butyl-2-(4-fluorophenyl)-1H-imidazol-5- |
| | yl]methyl}-2-(4-methoxyphenyl)ethanamine |
| 717. | N-(1,3-benzodioxol-5-ylmethyl)-N-{[1-butyl-2-(4-fluorophenyl)-1H-imidazol-5- |
| | yl]methyl}-2-(4-chlorophenyl)ethanamine |
| 718. | N-(1,3-benzodioxol-5-ylmethyl)-N-{[1-butyl-2-(3-fluorophenyl)-1H-imidazol-5- |
| | yl]methyl}-2-(3-methoxyphenyl)ethanamine |
| 719. | N-(1,3-benzodioxol-5-ylmethyl)-N-{[1-butyl-2-(3-fluorophenyl)-1H-imidazol-5- |
| | yl]methyl}-2-(4-methoxyphenyl)ethanamine |
| 720. | N-(1,3-benzodioxol-5-ylmethyl)-N-{[1-butyl-2-(4-fluorophenyl)-1H-imidazol-5- |
| | yl]methyl}-2-(4-iodophenyl)ethanamine |
| 721. | N-(1,3-benzodioxol-5-ylmethyl)-2-(4-bromophenyl)-N-{[1-butyl-2-(4-fluorophenyl)- |
| | 1H-imidazol-5-yl]methyl}ethanamine |
| 722. | N-(1,3-benzodioxol-5-ylmethyl)-N-{[1-butyl-2-(4-fluorophenyl)-1H-imidazol-5- |
| | yl]methyl}-2-[4-(methylthio)phenyl]ethanamine |
| 723. | N-(1,3-benzodioxol-5-ylmethyl)-N-{[1-butyl-2-(3-fluorophenyl)-1H-imidazol-5- |
| | yl]methyl}-2-(4-iodophenyl)ethanamine |
| 724. | N-(1,3-benzodioxol-5-ylmethyl)-N-{[1-butyl-2-(3-fluorophenyl)-1H-imidazol-5- |
| | yl]methyl}-2-[4-(methylthio)phenyl]ethanamine |
| 725. | N-(1,3-benzodioxol-5-ylmethyl)-2-[4-(methylthio)phenyl]-N-{[2-phenyl-1-(2- |
| | phenylethyl)-1H-imidazol-5-yl]methyl}ethanamine |
| 726. | 4-{[{[1-butyl-2-(3-fluorophenyl)-1H-imidazol-5-yl]methyl}(isopentyl)amino]methyl}- |
| | N-methylaniline |
| 727. | 4-{[{[1-butyl-2-(3-fluorophenyl)-1H-imidazol-5- |
| | yl]methyl}(cyclohexylmethyl)amino]methyl}-N-methylaniline |
| 728. | 3-{[{[1-butyl-2-(3-fluorophenyl)-1H-imidazol-5- |
| | yl]methyl}(cyclohexylmethyl)amino]methyl}-2-methylaniline |
| 729. | 1-(1,3-benzodioxol-5-yl)-N-{[1-butyl-2-(4-fluorophenyl)-1H-imidazol-5-yl]methyl}-N- |
| | (2,6-difluorobenzyl)methanamine |
| 730. | 1-(1,3-benzodioxol-5-yl)-N-{[1-butyl-2-(4-fluorophenyl)-1H-imidazol-5-yl]methyl}-N- |
| | [2-(methylthio)benzyl]methanamine |
| 731. | 1-(1,3-benzodioxol-5-yl)-N-{[1-butyl-2-(4-fluorophenyl)-1H-imidazol-5-yl]methyl}-N- |
| | [2-(trifluoromethoxy)benzyl]methanamine |
| 732. | 1-(1,3-benzodioxol-5-yl)-N-[2,5-bis(2,2,2-trifluoroethoxy)benzyl]-N-{[1-butyl-2-(4- |
| | fluorophenyl)-1H-imidazol-5-yl]methyl}methanamine |
| 733. | 1-(1,3-benzodioxol-5-yl)-N-(2-bromo-3-methylbenzyl)-N-{[1-butyl-2-(4-fluorophenyl)- |
| | 1H-imidazol-5-yl]methyl}methanamine |
| 734. | 1-(1,3-benzodioxol-5-yl)-N-{[1-butyl-2-(4-fluorophenyl)-1H-imidazol-5-yl]methyl}-N- |
| | [2-chloro-5-(methylthio)benzyl]methanamine |
| 735. | 1-(1,3-benzodioxol-5-yl)-N-{[1-butyl-2-(4-fluorophenyl)-1H-imidazol-5-yl]methyl}-N- |
| | (1-naphthylmethyl)methanamine |
| 736. | 1-(1,3-benzodioxol-5-yl)-N-{[1-butyl-2-(4-fluorophenyl)-1H-imidazol-5-yl]methyl}-N- |
| | (9H-fluoren-4-ylmethyl)methanamine |
| 737. | N-benzyl-1-[1-butyl-2-(3-fluorophenyl)-1H-imidazol-5-yl]-N-[2- |
| | (methylthio)benzyl]methanamine |
| 738. | 1-(1,3-benzodioxol-5-yl)-N-{[1-butyl-2-(3-fluorophenyl)-1H-imidazol-5-yl]methyl}-N- |
| | [2-(methylthio)benzyl]methanamine |
| 739. | N-{[1-butyl-2-(3-fluorophenyl)-1H-imidazol-5-yl]methyl}-N-(2-chloro-3,4- |
| | dimethoxybenzyl)-3-methylbutan-1-amine |
| 740. | N-{[1-butyl-2-(3-fluorophenyl)-1H-imidazol-5-yl]methyl}-N-(2-chloro-4,5- |
| | dimethoxybenzyl)-N-(cyclohexylmethyl)amine |
| 741. | 1-(1,3-benzodioxol-5-yl)-N-{[1-butyl-2-(4-fluorophenyl)-1H-imidazol-5-yl]methyl}-N- |
| | (2-naphthylmethyl)methanamine |
| 742. | N-{[1-butyl-2-(4-fluorophenyl)-1H-imidazol-5-yl]methyl}-N-(3-iodo-4-methylbenzyl)- |
| | 3-methylbutan-1-amine |
| 743. | N-{[1-butyl-2-(4-fluorophenyl)-1H-imidazol-5-yl]methyl}-N-(3-chloro-4- |
| | methylbenzyl)-3-methylbutan-1-amine |
| 744. | 1-(1,3-benzodioxol-5-yl)-N-{[1-butyl-2-(4-fluorophenyl)-1H-imidazol-5-yl]methyl}-N- |
| | (3-chloro-4-methylbenzyl)methanamine |
| 745. | N-{[1-butyl-2-(4-fluorophenyl)-1H-imidazol-5-yl]methyl}-N-(2,3-dihydro-1- |
| | benzofuran-5-ylmethyl)-3-methylbutan-1-amine |
| 746. | 1-(1,3-benzodioxol-5-yl)-N-{[1-butyl-2-(4-fluorophenyl)-1H-imidazol-5-yl]methyl}-N- |
| | (2,3-dihydro-1-benzofuran-5-ylmethyl)methanamine |
| 747. | N-{[1-butyl-2-(4-fluorophenyl)-1H-imidazol-5 -yl]methyl}-N-(cyclohexylmethyl)-N- |
| | (2,3-dihydro-1-benzofuran-5-ylmethyl)amine |
| 748. | 1-(1,3-benzodioxol-5-yl)-N-{[1-butyl-2-(4-fluorophenyl)-1H-imidazol-5-yl]methyl}-N- |
| | [4-(difluoromethoxy)benzyl]methanamine |
| 749. | N-{[1-butyl-2-(3-fluorophenyl)-1H-imidazol-5-yl]methyl}-3-methyl-N-(2- |
| | naphthylmethyl)butan-1-amine |
| 750. | N-{[1-butyl-2-(3-fluorophenyl)-1H-imidazol-5-yl]methyl}-N-(3-iodo-4-methylbenzyl)- |
| | 3-methylbutan-1-amine |
| 751. | N-{[1-butyl-2-(3-fluorophenyl)-1H-imidazol-5-yl]methyl}-N-(cyclohexylmethyl)-N-(3- |
| | iodo-4-methylbenzyl)amine |
| 752. | 1-(1,3-benzodioxol-5-yl)-N-{[1-butyl-2-(3-fluorophenyl)-1H-imidazol-5-yl]methyl}-N- |
| | (2,3-dihydro-1-benzofuran-5-ylmethyl)methanamine |
| 753. | N-{[1-butyl-2-(3-fluorophenyl)-1H-imidazol-5-yl]methyl}-N-(cyclohexylmethyl)-N- |
| | (2,3-dihydro-1-benzofuran-5-ylmethyl)amine |
| 754. | N-{[1-butyl-2-(3-fluorophenyl)-1H-imidazol-5-yl]methyl}-N-[4- |
| | (difluoromethoxy)benzyl]-3-methylbutan-1-amine |
| 755. | N-{[1-butyl-2-(3-fluorophenyl)-1H-imidazol-5-yl]methyl}-N-(cyclohexylmethyl)-N-[4- |
| | (difluoromethoxy)benzyl]amine |
| 756. | N-(3-iodo-4-methylbenzyl)-3-methyl-N-{[2-phenyl-1-(2-phenylethyl)-1H-imidazol-5- |
| | yl]methyl}butan-1-amine |
| 757. | N-(1,3-benzodioxol-5-ylmethyl)-N-{[1-butyl-2-(4-fluorophenyl)-1H-imidazol-5- |
| | yl]methyl}-2-(2-phenoxyphenyl)ethanamine |
| 758. | N-(1,3-benzodioxol-5-ylmethyl)-N-{[1-butyl-2-(4-fluorophenyl)-1H-imidazol-5- |
| | yl]methyl}-2-(2-fluorophenyl)ethanamine |
| 759. | N-(1,3-benzodioxol-5-ylmethyl)-N-{[1-butyl-2-(4-fluorophenyl)-1H-imidazol-5- |
| | yl]methyl}-2-(2-methoxyphenyl)ethanamine |
| 760. | N-(1,3-benzodioxol-5-ylmethyl)-N-{[1-butyl-2-(4-fluorophenyl)-1H-imidazol-5- |
| | yl]methyl}-2-(2-ethoxyphenyl)ethanamine |
| 761. | N-(1,3-benzodioxol-5-ylmethyl)-2-(2-bromophenyl)-N-{[1-butyl-2-(4-fluorophenyl)- |
| | 1H-imidazol-5-yl]methyl}ethanamine |
| 762. | N-(1,3-benzodioxol-5-ylmethyl)-N-{[1-butyl-2-(4-fluorophenyl)-1H-imidazol-5- |
| | yl]methyl}-2-(2,5-dimethoxyphenyl)ethanamine |
| 763. | N-(1,3-benzodioxol-5-ylmethyl)-N-{[1-butyl-2-(4-fluorophenyl)-1H-imidazol-5- |
| | yl]methyl}-2-(2,3-dimethoxyphenyl)ethanamine |
| 764. | N-(1,3-benzodioxol-5-ylmethyl)-N-{[1-butyl-2-(4-fluorophenyl)-1H-imidazol-5- |
| | yl]methyl}-2-(2,4-dimethoxyphenyl)ethanamine |
| 765. | N-(1,3-benzodioxol-5-ylmethyl)-N-{[1-butyl-2-(3-fluorophenyl)-1H-imidazol-5- |
| | yl]methyl}-2-(3,4-dimethoxyphenyl)ethanamine |
| 766. | N-(1,3-benzodioxol-5-ylmethyl)-N-{[1-butyl-2-(3-fluorophenyl)-1H-imidazol-5- |
| | yl]methyl}-2-(4-ethoxy-3-methoxyphenyl)ethanamine |
| 767. | N-(1,3-benzodioxol-5-ylmethyl)-N-{[1-butyl-2-(3-fluorophenyl)-1H-imidazol-5- |
| | yl]methyl}-2-(2-naphthyl)ethanamine |
| 768. | 1-(1,3-benzodioxol-5-yl)-N-{[1-butyl-2-(4-fluorophenyl)-1H-imidazol-5-yl]methyl}-N- |
| | {[6-methyl-2-(4-methylphenoxy)pyridin-3-yl]methyl}methanamine |
| 769. | 1-(1,3-benzodioxol-5-yl)-N-{[1-butyl-2-(4-fluorophenyl)-1H-imidazol-5-yl]methyl}-N- |
| | [(2-phenoxypyridin-3-yl)methyl]methanamine |
| 770. | N-{[2-(allylthio)pyridin-3-yl]methyl}-N-(1,3-benzodioxol-5-ylmethyl)-N-{[1-butyl-2- |
| | (4-fluorophenyl)-1H-imidazol-5-yl]methyl}amine |
| 771. | 2-chloro-N-{[1-(2,3-dihydro-1H-indan-2-yl)-2-phenyl-1H-imidazol-5-yl]methyl}-N-(4- |
| | methoxybenzyl)benzamide |
| 772. | N-{[1-(2,3-dihydro-1H-indan-2-yl)-2-phenyl-1H-imidazol-5-yl]methyl}-2,5-difluoro- |
| | N-(4-methoxybenzyl)benzamide |
| 773. | N-(cyclohexylmethyl)-N-{[1-(2,3-dihydro-1H-indan-2-yl)-2-phenyl-1H-imidazol-5- |
| | yl]methyl}-2,5-difluorobenzamide |
| 774. | 5-chloro-N-{[1-(2,3-dihydro-1H-indan-2-yl)-2-phenyl-1H-imidazol-5-yl]methyl}-2- |
| | methoxy-N-(4-methoxybenzyl)benzamide |
| 775. | 5-chloro-N-(cyclohexylmethyl)-N-{[1-(2,3-dihydro-1H-indan-2-yl)-2-phenyl-1H- |
| | imidazol-5-yl]methyl}-2-methoxybenzamide |
| 776. | N-{[1-(2,3-dihydro-1H-indan-2-yl)-2-phenyl-1H-imidazol-5-yl]methyl}-N-(4- |
| | methoxybenzyl)-2-(trifluoromethyl)benzamide |
| 777. | N-benzyl-2,5-dichloro-N-{[1-(2,3-dihydro-1H-indan-2-yl)-2-phenyl-1H-imidazol-5- |
| | yl]methyl}benzamide |
| 778. | 2,5-dichloro-N-{[1-(2,3-dihydro-1H-indan-2-yl)-2-phenyl-1H-imidazol-5-yl]methyl}- |
| | N-(4-methoxybenzyl)benzamide |
| 779. | 2,5-dichloro-N-(cyclohexylmethyl)-N-{[1-(2,3-dihydro-1H-indan-2-yl)-2-phenyl-1H- |
| | imidazol-5-yl]methyl}benzamide |
| 780. | 2-bromo-N-{[1-(2,3-dihydro-1H-indan-2-yl)-2-phenyl-1H-imidazol-5-yl]methyl}-N-(4- |
| | methoxybenzyl)benzamide |
| 781. | N-{[1-(2,3-dihydro-1H-indan-2-yl)-2-phenyl-1H-imidazol-5-yl]methyl}-N-(4- |
| | methoxybenzyl)-2-(2-phenylethyl)benzamide |
| 782. | N-{[1-(2,3-dihydro-1H-indan-2-yl)-2-phenyl-1H-imidazol-5-yl]methyl}-2-iodo-N-(4- |
| | methoxybenzyl)benzamide |
| 783. | 3-chloro-N-{[1-(2,3-dihydro-1H-indan-2-yl)-2-phenyl-1H-imidazol-5-yl]methyl}-2,6- |
| | dimethoxy-N-(4-methoxybenzyl)benzamide |
| 784. | 2-bromo-N-{[1-(2,3-dihydro-1H-indan-2-yl)-2-phenyl-1H-imidazol-5-yl]methyl}-5- |
| | methoxy-N-(4-methoxybenzyl)benzamide |
| 785. | 3-bromo-N-{[1-(2,3-dihydro-1H-indan-2-yl)-2-phenyl-1H-imidazol-5-yl]methyl}-2,6- |
| | dimethoxy-N-(4-methoxybenzyl)benzamide |
| 786. | N-{[1-(2,3-dihydro-1H-indan-2-yl)-2-phenyl-1H-imidazol-5-yl]methyl}-2-fluoro-6- |
| | iodo-N-(4-methoxybenzyl)benzamide |
| 787. | 2-bromo-N-{[1-(2,3-dihydro-1H-indan-2-yl)-2-phenyl-1H-imidazol-5-yl]methyl}-N-(4- |
| | methoxybenzyl)-5-methylbenzamide |
| 788. | 2-bromo-N-(cyclohexylmethyl)-N-{[1-(2,3-dihydro-1H-indan-2-yl)-2-phenyl-1H- |
| | imidazol-5-yl]methyl}-5-methylbenzamide |
| 789. | 2-chloro-N-{[1-(2,3-dihydro-1H-indan-2-yl)-2-phenyl-1H-imidazol-5-yl]methyl}-N-(4- |
| | methoxybenzyl)-5-(methylthio)benzamide |
| 790. | 2-chloro-N-{[1-(2,3-dihydro-1H-indan-2-yl)-2-phenyl-1H-imidazol-5-yl]methyl}-N-(4- |
| | methoxybenzyl)-5-(trifluoromethyl)benzamide |
| 791. | 2-chloro-N-(cyclohexylmethyl)-N-{[1-(2,3-dihydro-1H-indan-2-yl)-2-phenyl-1H- |
| | imidazol-5-yl]methyl}-5-(trifluoromethyl)benzamide |
| 792. | N-{[1-(2,3-dihydro-1H-indan-2-yl)-2-phenyl-1H-imidazol-5-yl]methyl}-N-(4- |
| | methoxybenzyl)-2-(1H-pyrrol-1-yl)benzamide |
| 793. | N-benzyl-1-(1-butyl-2,4-diphenyl-1H-imidazol-5-yl)-N-(4- |
| | methoxybenzyl)methanamine |
| 794. | N-benzyl-N-[(4-bromo-1-butyl-2-phenyl-1H-imidazol-5-yl)methyl]butan-1-amine |
| 795. | N-[(4-bromo-1-butyl-2-phenyl-1H-imidazol-5-yl)methyl]-N-(2,3-dichlorobenzyl)butan- |
| | 1-amine |
| 796. | N-benzyl-N-[(1-butyl-4-chloro-2-phenyl-1H-imidazol-5-yl)methyl]butan-1-amine |
| 797. | N-benzyl-N-[(1-butyl-2,4-diphenyl-1H-imidazol-5-yl)methyl]butan-1-amine |
| 798. | N-benzyl-N-[(1-butyl-4-methyl-2-phenyl-1H-imidazol-5-yl)methyl]butan-1-amine |
| 799. | 1-(1,3-benzodioxol-5-yl)-N-benzyl-N-[(5-butyl-1-phenyl-1H-imidazol-4- |
| | yl)methyl]methanamine |
| 800. | 4-{[[(1-butyl-2,4-diphenyl-1H-imidazol-5-yl)methyl](isopentyl)amino]methyl}-3- |
| | chlorophenol |
| 801. | 4-({butyl[(1-butyl-2,4-diphenyl-1H-imidazol-5-yl)methyl]amino}methyl)-3- |
| | chlorophenol |
| 802. | 4-({benzyl[(1-butyl-2,4-diphenyl-1H-imidazol-5-yl)methyl]amino}methyl)-3- |
| | chlorophenol |
| 803. | 4-({butyl[(1-butyl-4-tert-butyl-2-phenyl-1H-imidazol-5-yl)methyl]amino}methyl)-3- |
| | chlorophenol |
| 804. | 1-(1,3-benzodioxol-5-yl)-N-benzyl-N-[(1-butyl-4-tert-butyl-2-phenyl-1H-imidazol-5- |
| | yl)methyl]methanamine |
| 805. | 1-(1,3-benzodioxol-5-yl)-N-(1,3-benzodioxol-5-ylmethyl)-N-[(1-butyl-4-tert-butyl-2- |
| | phenyl-1H-imidazol-5-yl)methyl]methanamine |
| 806. | 4-({(1,3-benzodioxol-5-ylmethyl)[(1-butyl-4-tert-butyl-2-phenyl-1H-imidazol-5- |
| | yl)methyl]amino}methyl)-3-chlorophenol |
| 807. | 4-({(1,3-benzodioxol-5-ylmethyl)[(1-butyl-4-tert-butyl-2-phenyl-1H-imidazol-5- |
| | yl)methyl]amino}methyl)-2-chlorophenol |
| 808. | 4-[(butyl{[1-butyl-4-(4-methoxyphenyl)-2-phenyl-1H-imidazol-5- |
| | yl]methyl}amino)methyl]-3-chlorophenol |
| 809. | 2-{[5-({butyl[(1-butyl-2,4-diphenyl-1H-imidazol-5-yl)methyl]amino}methyl)pyridin-2- |
| | yl]amino}ethanol |
| 810. | 4-[(butyl{[1-butyl-4-(4-methoxyphenyl)-2-phenyl-1H-imidazol-5- |
| | yl]methyl}amino)methyl]phenol |
| 811. | 4-[(benzyl{[1-butyl-4-(4-methoxyphenyl)-2-phenyl-1H-imidazol-5- |
| | yl]methyl}amino)methyl]phenol |
| 812. | N-(1,3-benzodioxol-5-ylmethyl)-N-{[1-butyl-4-(4-methoxyphenyl)-2-phenyl-1H- |
| | imidazol-5-yl]methyl}butan-1-amine |
| 813. | N-benzyl-N-{[1-butyl-4-(4-fluorophenyl)-2-phenyl-1H-imidazol-5-yl]methyl}butan-1- |
| | amine |
| 814. | 4-[(butyl{[1-butyl-4-(4-fluorophenyl)-2-phenyl-1H-imidazol-5- |
| | yl]methyl}amino)methyl]-3-chlorophenol |
| 815. | 4-[(benzyl{[1-butyl-4-(4-fluorophenyl)-2-phenyl-1H-imidazol-5- |
| | yl]methyl}amino)methyl]-3-chlorophenol |
| 816. | N-(1,3-benzodioxol-5-ylmethyl)-N-{[1-butyl-4-(4-fluorophenyl)-2-phenyl-1H- |
| | imidazol-5-yl]methyl}butan-1-amine |
| 817. | 1-(1,3-benzodioxol-5-yl)-N-benzyl-N-{[1-butyl-4-(4-fluorophenyl)-2-phenyl-1H- |
| | imidazol-5-yl]methyl}methanamine |
| 818. | N-[(1-butyl-2,4-diphenyl-1H-imidazol-5-yl)methyl]-N-[(6-chloropyridin-3- |
| | yl)methyl]butan-1-amine |
| 819. | N-benzyl-1-(1-butyl-2,4-diphenyl-1H-imidazol-5-yl)-N-[(6-chloropyridin-3- |
| | yl)methyl]methanamine |
| 820. | N-[(1-butyl-2,4-diphenyl-1H-imidazol-5-yl)methyl]-N-[(6-pyrrolidin-1-ylpyridin-3- |
| | yl)methyl]butan-1-amine |
| 821. | N-benzyl-1-(1-butyl-2,4-diphenyl-1H-imidazol-5-yl)-N-[(6-pyrrolidin-1-ylpyridin-3- |
| | yl)methyl]methanamine |
| 822. | 4-[(benzyl{[1-butyl-4-(4-methoxyphenyl)-2-phenyl-1H-imidazol-5- |
| | yl]methyl}amino)methyl]-3-chlorophenol |
| 823. | 2-{[5-({benzyl[(1-butyl-2,4-diphenyl-1H-imidazol-5-yl)methyl]amino}methyl)pyridin- |
| | 2-yl]amino}ethanol |
| 824. | 4-({butyl[(1-butyl-2,4-diphenyl-1H-imidazol-5-yl)methyl]amino}methyl)benzene-1,3- |
| | diol |
| 825. | 4-({benzyl[(1-butyl-2,4-diphenyl-1H-imidazol-5-yl)methyl]amino}methyl)phenol |
| 826. | 4-({butyl[(1-butyl-2,4-diphenyl-1H-imidazol-5-yl)methyl]amino}methyl)phenol |
| 827. | N-benzyl-1-(1-butyl-2,4-diphenyl-1H-imidazol-5-yl)-N-(2,3-dihydro-1-benzofuran-5- |
| | ylmethyl)methanamine |
| 828. | N-[(1-butyl-2,4-diphenyl-1H-imidazol-5-yl)methyl]-N-(2,3-dihydro-1-benzofuran-5- |
| | ylmethyl)butan-1-amine |
| 829. | N-benzyl-1-(1-butyl-2,4-diphenyl-1H-imidazol-5-yl)-N-(2,3-dihydro-1,4-benzodioxin- |
| | 6-ylmethyl)methanamine |
| 830. | N-[(1-butyl-2,4-diphenyl-1H-imidazol-5-yl)methyl]-N-(2,3-dihydro-1,4-benzodioxin-6- |
| | ylmethyl)butan-1-amine |
| 831. | 1-(1,3-benzodioxol-5-yl)-N-benzyl-N-[(1-butyl-2,4-diphenyl-1H-imidazol-5- |
| | yl)methyl]methanamine |
| 832. | N-(1,3-benzodioxol-5-ylmethyl)-N-[(1-butyl-2,4-diphenyl-1H-imidazol-5- |
| | yl)methyl]butan-1-amine |
| 833. | N-benzyl-N-[(1-butyl-2,4-diphenyl-1H-imidazol-5-yl)methyl]ethanamine |
| 834. | 5-({butyl[(1-butyl-2,4-diphenyl-1H-imidazol-5-yl)methyl]amino}methyl)pyridin-2- |
| | amine |
| 835. | N-[(1-butyl-2,4-diphenyl-1H-imidazol-5-yl)methyl]-N-(1,3-thiazol-2-ylmethyl)butan-1- |
| | amine |
| 836. | N-benzyl-1-(1-butyl-2,4-diphenyl-1H-imidazol-5-yl)-N-(1,3-thiazol-2- |
| | ylmethyl)methanamine |
| 837. | N-benzyl-N-[(2,4-diphenyl-1-propyl-1H-imidazol-5-yl)methyl]butan-1-amine |
| 838. | ethyl N-benzyl-N-[(1-butyl-2,4-diphenyl-1H-imidazol-5-yl)methyl]glycinate |
| 839. | 2-({benzyl[(1-butyl-2,4-diphenyl-1H-imidazol-5-yl)methyl]amino}methyl)phenol |
| 840. | 3-({benzyl[(1-butyl-2,4-diphenyl-1H-imidazol-5-yl)methyl]amino}methyl)phenol |
| 841. | 2-({butyl[(1-butyl-2,4-diphenyl-1H-imidazol-5-yl)methyl]amino}methyl)phenol |
| 842. | 3-({butyl[(1-butyl-2,4-diphenyl-1H-imidazol-5-yl)methyl]amino}methyl)phenol |
| 843. | 5-({butyl[(1-butyl-2,4-diphenyl-1H-imidazol-5-yl)methyl]amino}methyl)-N-(2- |
| | methoxyethyl)pyridin-2-amine |
| 844. | 5-({butyl[(1-butyl-2,4-diphenyl-1H-imidazol-5-yl)methyl]amino}methyl)-N- |
| | methylpyridin-2-amine |
| 845. | 5-({butyl[(1-butyl-2,4-diphenyl-1H-imidazol-5-yl)methyl]amino}methyl)-N- |
| | ethylpyridin-2-amine |
| 846. | methyl 4-({benzyl[(1-butyl-2,4-diphenyl-1H-imidazol-5- |
| | yl)methyl]amino}methyl)benzoate |
| 847. | methyl 4-({butyl[(1-butyl-2,4-diphenyl-1H-imidazol-5- |
| | yl)methyl]amino}methyl)benzoate |
| 848. | N-benzyl-1-(1-butyl-2,4-diphenyl-1H-imidazol-5-yl)-N-(quinolin-3- |
| | ylmethyl)methanamine |
| 849. | N-[(1-butyl-2,4-diphenyl-1H-imidazol-5-yl)methyl]-N-(quinolin-3-ylmethyl)butan-1- |
| | amine |
| 850. | 1-(1-butyl-2,4-diphenyl-1H-imidazol-5-yl)-N-(cyclohexylmethyl)-N-(quinolin-3- |
| | ylmethyl)methanamine |
| 851. | N-[(1-butyl-2,4-diphenyl-1H-imidazol-5-yl)methyl]-N-(4-methoxybenzyl)butan-1- |
| | amine |
| 852. | 5-({benzyl[(1-butyl-2,4-diphenyl-1H-imidazol-5-yl)methyl]amino}methyl)-N-(2- |
| | methoxyethyl)pyridin-2-amine |
| 853. | 4-({benzyl[(1-butyl-2,4-diphenyl-1H-imidazol-5-yl)methyl]amino}methyl)benzoic acid |
| 854. | 4-({butyl[(1-butyl-2,4-diphenyl-1H-imidazol-5-yl)methyl]amino}methyl)benzoic acid |
| 855. | 4-{[[(1-butyl-2,4-diphenyl-1H-imidazol-5- |
| | yl)methyl](cyclohexylmethyl)amino]methyl}benzoic acid |
| 856. | N-[(1-butyl-2,4-diphenyl-1H-imidazol-5-yl)methyl]-N-(quinolin-2-ylmethyl)butan-1- |
| | amine |
| 857. | 1-(1-butyl-2,4-diphenyl-1H-imidazol-5-yl)-N-(cyclohexylmethyl)-N-(quinolin-2- |
| | ylmethyl)methanamine |
| 858. | 5-({benzyl[(1-butyl-2,4-diphenyl-1H-imidazol-5-yl)methyl]amino}methyl)-N- |
| | ethylpyridin-2-amine |
| 859. | 1-(4-bromophenyl)-N-[(1-butyl-2,4-diphenyl-1H-imidazol-5-yl)methyl]-N- |
| | (cyclohexylmethyl)methanamine |
| 860. | 4-({benzyl[(1-butyl-2,4-diphenyl-1H-imidazol-5-yl)methyl]amino}methyl)-N,N- |
| | dimethylaniline |
| 861. | 4-({butyl[(1-butyl-2,4-diphenyl-1H-imidazol-5-yl)methyl]amino}methyl)-N,N- |
| | dimethylaniline |
| 862. | N-[(1-butyl-2,4-diphenyl-1H-imidazol-5-yl)methyl]-N-(cyclohexylmethyl)-N-[4- |
| | (dimethylamino)benzyl]amine |
| 863. | N-(1H-benzimidazol-5-ylmethyl)-N-[(1-butyl-2,4-diphenyl-1H-imidazol-5- |
| | yl)methyl]butan-1-amine |
| 864. | N-benzyl-1-(1-butyl-2,4-diphenyl-1H-imidazol-5-yl)-N-[4-(1,3-thiazol-5- |
| | yl)benzyl]methanamine |
| 865. | N-[(1-butyl-2,4-diphenyl-1H-imidazol-5-yl)methyl]-N-[4-(1,3-thiazol-5- |
| | yl)benzyl]butan-1-amine |
| 866. | 1-(1-butyl-2,4-diphenyl-1H-imidazol-5-yl)-N-(cyclohexylmethyl)-N-[4-(1,3-thiazol-5- |
| | yl)benzyl]methanamine |
| 867. | 4-({benzyl[(1-butyl-2,4-diphenyl-1H-imidazol-5-yl)methyl]amino}methyl)-N- |
| | methylbenzamide |
| 868. | 4-({butyl[(1-butyl-2,4-diphenyl-1H-imidazol-5-yl)methyl]amino}methyl)-N- |
| | methylbenzamide |
| 869. | 4-({butyl[(1-butyl-2,4-diphenyl-1H-imidazol-5-yl)methyl]amino}methyl)-N,N- |
| | dimethylbenzamide |
| 870. | 4-{[[(1-butyl-2,4-diphenyl-1H-imidazol-5- |
| | yl)methyl](cyclohexylmethyl)amino]methyl}-N,N-dimethylbenzamide |
| 871. | [4-({benzyl[(1-butyl-2,4-diphenyl-1H-imidazol-5- |
| | yl)methyl]amino}methyl)phenyl]methanol |
| 872. | [4-({butyl[(1-butyl-2,4-diphenyl-1H-imidazol-5- |
| | yl)methyl]amino}methyl)phenyl]methanol |
| 873. | (4-{[[(1-butyl-2,4-diphenyl-1H-imidazol-5- |
| | yl)methyl](cyclohexylmethyl)amino]methyl}phenyl)methanol |
| 874. | N-benzyl-1-(1-butyl-2,4-diphenyl-1H-imidazol-5-yl)-N-(2- |
| | methoxybenzyl)methanamine |
| 875. | N-[(1-butyl-2,4-diphenyl-1H-imidazol-5-yl)methyl]-N-(2-methoxybenzyl)butan-1- |
| | amine |
| 876. | 1-(1-butyl-2,4-diphenyl-1H-imidazol-5-yl)-N-(cyclohexylmethyl)-N-(2- |
| | methoxybenzyl)methanamine |
| 877. | N-benzyl-1-(1-butyl-2,4-diphenyl-1H-imidazol-5-yl)-N-(3- |
| | methoxybenzyl)methanamine |
| 878. | N-[(1-butyl-2,4-diphenyl-1H-imidazol-5-yl)methyl]-N-(3-methoxybenzyl)butan-1- |
| | amine |
| 879. | 1-(1-butyl-2,4-diphenyl-1H-imidazol-5-yl)-N-(cyclohexylmethyl)-N-(3- |
| | methoxybenzyl)methanamine |
| 880. | 4-{[[(1-butyl-2,4-diphenyl-1H-imidazol-5- |
| | yl)methyl](cyclohexylmethyl)amino]methyl}phenyl acetate |
| 881. | methyl 4-({butyl[(1-butyl-4-tert-butyl-2-phenyl-1H-imidazol-5- |
| | yl)methyl]amino}methyl)benzoate |
| 882. | [4-({butyl[(1-butyl-4-tert-butyl-2-phenyl-1H-imidazol-5- |
| | yl)methyl]amino}methyl)phenyl]methanol |
| 883. | [4-({benzyl[(1-butyl-2,4-diphenyl-1H-imidazol-5- |
| | yl)methyl]amino}methyl)phenoxy]acetic acid |
| 884. | (4-{[[(1-butyl-2,4-diphenyl-1H-imidazol-5- |
| | yl)methyl](cyclohexylmethyl)amino]methyl}phenoxy)acetic acid |
| 885. | 2-[4-({benzyl[(1-butyl-2,4-diphenyl-1H-imidazol-5- |
| | yl)methyl]amino}methyl)phenoxy]ethanol |
| 886. | 2-[4-({butyl[(1-butyl-2,4-diphenyl-1H-imidazol-5- |
| | yl)methyl]amino}methyl)phenoxy]ethanol |
| 887. | 2-(4-{[[(1-butyl-2,4-diphenyl-1H-imidazol-5- |
| | yl)methyl](cyclohexylmethyl)amino]methyl}phenoxy)ethanol |
| 888. | 4-({butyl[(1-butyl-4-tert-butyl-2-phenyl-1H-imidazol-5-yl)methyl]amino}methyl)-N- |
| | methylbenzamide |
| 889. | 4-({benzyl[(1-butyl-2,4-diphenyl-1H-imidazol-5-yl)methyl]amino}methyl)-2- |
| | nitrophenol |
| 890. | 4-({butyl[(1-butyl-2,4-diphenyl-1H-imidazol-5-yl)methyl]amino}methyl)-2-nitrophenol |
| 891. | 4-{[[(1-butyl-2,4-diphenyl-1H-imidazol-5- |
| | yl)methyl](cyclohexylmethyl)amino]methyl}-2-nitrophenol |
| 892. | 5-({benzyl[(1-butyl-2,4-diphenyl-1H-imidazol-5-yl)methyl]amino}methyl)-2- |
| | nitrophenol |
| 893. | 5-({butyl[(1-butyl-2,4-diphenyl-1H-imidazol-5-yl)methyl]amino}methyl)-2-nitrophenol |
| 894. | 5-{[[(1-butyl-2,4-diphenyl-1H-imidazol-5- |
| | yl)methyl](cyclohexylmethyl)amino]methyl}-2-nitrophenol |
| 895. | N,N-dibutyl-4-({butyl[(1-butyl-2,4-diphenyl-1H-imidazol-5- |
| | yl)methyl]amino}methyl)aniline |
| 896. | N,N-dibutyl-4-{[[(1-butyl-2,4-diphenyl-1H-imidazol-5- |
| | yl)methyl](cyclohexylmethyl)amino]methyl}aniline |
| 897. | N-[(1-butyl-2,4-diphenyl-1H-imidazol-5-yl)methyl]-N-{4-[3- |
| | (dimethylamino)propoxy]benzyl}butan-1-amine |
| 898. | 3-(4-{[[(1-butyl-2,4-diphenyl-1H-imidazol-5- |
| | yl)methyl](cyclohexylmethyl)amino]methyl}phenoxy)-N,N-dimethylpropan-1-amine |
| 899. | 4-({butyl[(1-butyl-4-tert-butyl-2-phenyl-1H-imidazol-5- |
| | yl)methyl]amino}methyl)benzamide |
| 900. | methyl 2-({benzyl[(1-butyl-2,4-diphenyl-1H-imidazol-5- |
| | yl)methyl]amino}methyl)benzoate |
| 901. | methyl 2-({butyl[(1-butyl-2,4-diphenyl-1H-imidazol-5- |
| | yl)methyl]amino}methyl)benzoate |
| 902. | methyl 2-{[[(1-butyl-2,4-diphenyl-1H-imidazol-5- |
| | yl)methyl](cyclohexylmethyl)amino]methyl}benzoate |
| 903. | [2-({benzyl[(1-butyl-2,4-diphenyl-1H-imidazol-5- |
| | yl)methyl]amino}methyl)phenyl]methanol |
| 904. | [2-({butyl[(1-butyl-2,4-diphenyl-1H-imidazol-5- |
| | yl)methyl]amino}methyl)phenyl]methanol |
| 905. | (2-{[[(1-butyl-2,4-diphenyl-1H-imidazol-5- |
| | yl)methyl](cyclohexylmethyl)amino]methyl}phenyl)methanol |
| 906. | 4-({benzyl[(1-butyl-2,4-diphenyl-1H-imidazol-5-yl)methyl]amino}methyl)phenyl |
| | glycinate |
| 907. | N-[4-(aminomethyl)benzyl]-N-[(1-butyl-4-tert-butyl-2-phenyl-1H-imidazol-5- |
| | yl)methyl]butan-1-amine |
| 908. | 4-({benzyl[(1-butyl-2,4-diphenyl-1H-imidazol-5- |
| | yl)methyl]amino}methyl)benzenesulfonamide |
| 909. | 4-({butyl[(1-butyl-2,4-diphenyl-1H-imidazol-5- |
| | yl)methyl]amino}methyl)benzenesulfonamide |
| 910. | N-{[1-butyl-4-(2-methoxyphenyl)-2-phenyl-1H-imidazol-5-yl]methyl}-N-(4- |
| | methoxybenzyl)butan-1-amine |
| 911. | N-{[1-butyl-4-(2-methoxyphenyl)-2-phenyl-1H-imidazol-5-yl]methyl}-N-[4- |
| | (difluoromethoxy)benzyl]butan-1-amine |
| 912. | methyl 6-{benzyl[(1-butyl-2,4-diphenyl-1H-imidazol-5-yl)methyl]amino}hexanoate |
| 913. | methyl 6-{butyl[(1-butyl-2,4-diphenyl-1H-imidazol-5-yl)methyl]amino}hexanoate |
| 914. | 6-{benzyl[(1-butyl-2,4-diphenyl-1H-imidazol-5-yl)methyl]amino}hexanoic acid |
| 915. | 6-{benzyl[(1-butyl-2,4-diphenyl-1H-imidazol-5-yl)methyl]amino}hexan-1-ol |
| 916. | 6-{butyl[(1-butyl-2,4-diphenyl-1H-imidazol-5-yl)methyl]amino}hexan-1-ol |
| 917. | methyl 3-({benzyl[(1-butyl-2,4-diphenyl-1H-imidazol-5- |
| | yl)methyl]amino}methyl)benzoate |
| 918. | methyl 3-({butyl[(1-butyl-2,4-diphenyl-1H-imidazol-5- |
| | yl)methyl]amino}methyl)benzoate |
| 919. | methyl 3-{[[(1-butyl-2,4-diphenyl-1H-imidazol-5- |
| | yl)methyl](cyclohexylmethyl)amino]methyl}benzoate |
| 920. | 1-(1,3-benzodioxol-5-yl)-N-(1,3-benzodioxol-5-ylmethyl)-N-[(1-butyl-2,4-diphenyl- |
| | 1H-imidazol-5-yl)methyl]methanamine |
| 921. | methyl 4-({(1,3-benzodioxol-5-ylmethyl)[(1-butyl-2,4-diphenyl-1H-imidazol-5- |
| | yl)methyl]amino}methyl)benzoate |
| 922. | 4-({(1,3-benzodioxol-5-ylmethyl)[(1-butyl-2,4-diphenyl-1H-imidazol-5- |
| | yl)methyl]amino}methyl)benzoic acid |
| 923. | N-(1,3-benzodioxol-5-ylmethyl)-N-benzyl-1-(1-butyl-2,4-diphenyl-1H-imidazol-5- |
| | yl)ethanamine |
| 924. | N-(1,3-benzodioxol-5-ylmethyl)-N-benzyl-1-(1-butyl-2,4-diphenyl-1H-imidazol-5- |
| | yl)pentan-1-amine |
| 925. | N-[4-({butyl[(1-butyl-2,4-diphenyl-1H-imidazol-5-yl)methyl]amino}methyl)phenyl]-N- |
| | (methylsulfonyl)methanesulfonamide |
| 926. | N-[4-({butyl[(1-butyl-2,4-diphenyl-1H-imidazol-5- |
| | yl)methyl]amino}methyl)phenyl]methanesulfonamide |
| 927. | 4-({(1,3-benzodioxol-5-ylmethyl)[(1-butyl-2,4-diphenyl-1H-imidazol-5- |
| | yl)methyl]amino}methyl)benzamide |
| 928. | 4-({butyl[(1-butyl-2,4-diphenyl-1H-imidazol-5-yl)methyl]amino}methyl)benzamide |
| 929. | 3-({benzyl[(1-butyl-2,4-diphenyl-1H-imidazol-5-yl)methyl]amino}methyl)benzoic acid |
| 930. | 3-{[[(1-butyl-2,4-diphenyl-1H-imidazol-5- |
| | yl)methyl](cyclohexylmethyl)amino]methyl}benzoic acid |
| 931. | [3-({benzyl[(1-butyl-2,4-diphenyl-1H-imidazol-5- |
| | yl)methyl]amino}methyl)phenyl]methanol |
| 932. | [3-({butyl[(1-butyl-2,4-diphenyl-1H-imidazol-5- |
| | yl)methyl]amino}methyl)phenyl]methanol |
| 933. | methyl 5-({benzyl[(1-butyl-2,4-diphenyl-1H-imidazol-5-yl)methyl]amino}methyl)-2- |
| | hydroxybenzoate |
| 934. | methyl 5-({butyl[(1-butyl-2,4-diphenyl-1H-imidazol-5-yl)methyl]amino}methyl)-2- |
| | hydroxybenzoate |
| 935. | methyl 5-{[[(1-butyl-2,4-diphenyl-1H-imidazol-5- |
| | yl)methyl](cyclohexylmethyl)amino]methyl}-2-hydroxybenzoate |
| 936. | 5-({benzyl[(1-butyl-2,4-diphenyl-1H-imidazol-5-yl)methyl]amino}methyl)-2- |
| | hydroxybenzoic acid |
| 937. | 5-{[[(1-butyl-2,4-diphenyl-1H-imidazol-5- |
| | yl)methyl](cyclohexylmethyl)amino]methyl}-2-hydroxybenzoic acid |
| 938. | 4-({benzyl[(1-butyl-2,4-diphenyl-1H-imidazol-5-yl)methyl]amino}methyl)-2- |
| | (hydroxymethyl)phenol |
| 939. | 4-({butyl[(1-butyl-2,4-diphenyl-1H-imidazol-5-yl)methyl]amino}methyl)-2- |
| | (hydroxymethyl)phenol |
| 940. | 4-{[[(1-butyl-2,4-diphenyl-1H-imidazol-5- |
| | yl)methyl](cyclohexylmethyl)amino]methyl}-2-(hydroxymethyl)phenol |
| 941. | N-[4-({butyl[(1-butyl-2,4-diphenyl-1H-imidazol-5- |
| | yl)methyl]amino}methyl)benzoyl]methanesulfonamide |
| 942. | N-{[1-butyl-4-(4-methoxyphenyl)-2-Phenyl-1H-imidazol-5-yl]methyl}-N-[4- |
| | (difluoromethoxy)benzyl]butan-1-amine |
| 943. | N-({1-butyl-4-[4-(2-chloroethoxy)phenyl]-2-phenyl-1H-imidazol-5-yl}methyl)-N-[4- |
| | (difluoromethoxy)benzyl]butan-1-amine |
| 944. | 3-({butyl[(1-butyl-4-{4-[2-(dimethylamino)ethoxy]phenyl}-2-phenyl-1H-imidazol-5- |
| | yl)methyl]amino}methyl)phenol |
| 945. | 4-({butyl[(1-butyl-4-{4-[2-(dimethylamino)ethoxy]phenyl}-2-phenyl-1H-imidazol-5- |
| | yl)methyl]amino}methyl)-3-chlorophenol |
| 946. | N-(1,3-benzodioxol-5-ylmethyl)-N[(1-butyl-4-{4-[2-(dimethylamino)ethoxy]phenyl}- |
| | 2-phenyl-1H-imidazol-5-yl)methyl]butan-1-amine |
| 947. | methyl 5-({benzyl[(1-butyl-2,4-diphenyl-1H-imidazol-5-yl)methyl]amino}methyl)-2- |
| | methoxybenzoate |
| 948. | methyl 5-({butyl[(1-butyl-2,4-diphenyl-1H-imidazol-5-yl)methyl]amino}methyl)-2- |
| | methoxybenzoate |
| 949. | methyl 5-{[[(1-butyl-2,4-diphenyl-1H-imidazol-5- |
| | yl)methyl](cyclohexylmethyl)amino]methyl}-2-methoxybenzoate |
| 950. | [5-({benzyl[(1-butyl-2,4-diphenyl-1H-imidazol-5-yl)methyl]amino}methyl)-2- |
| | methoxyphenyl]methanol |
| 951. | [5-({butyl[(1-butyl-2,4-diphenyl-1H-imidazol-5-yl)methyl]amino}methyl)-2- |
| | methoxyphenyl]methanol |
| 952. | (5-{[[(1-butyl-2,4-diphenyl-1H-imidazol-5- |
| | yl)methyl](cyclohexylmethyl)amino]methyl}-2-methoxyphenyl)methanol |
| 953. | N-[4-({benzyl[(1-butyl-2,4-diphenyl-1H-imidazol-5- |
| | yl)methyl]amino}methyl)benzoyl]methanesulfonamide |
| 954. | 4-({benzyl[(1-butyl-2,4-diphenyl-1H-imidazol-5-yl)methyl]amino}methyl)benzamide |
| 955. | N-[4-({butyl[(1-butyl-2,4-diphenyl-1H-imidazol-5-yl)methyl]amino}methyl)benzoyl]- |
| | 1,1,1-trifluoromethanesulfonamide |
| 956. | N-[(1-butyl-2,4-diphenyl-1H-imidazol-5-yl)methyl]-N-{4- |
| | [(methylthio)methoxy]benzyl}butan-1-amine |
| 957. | N-benzyl-1-(1-butyl-2,4-diphenyl-1H-imidazol-5-yl)-N-{4- |
| | [(methylthio)methoxy]benzyl}methanamine |
| 958. | 4-({(1,3-benzodioxol-5-ylmethyl)[(1-butyl-2,4-diphenyl-1H-imidazol-5- |
| | yl)methyl]amino}methyl)benzenesulfonamide |
| 959. | 4-{[[(1-butyl-2,4-diphenyl-1H-imidazol-5-yl)methyl](4- |
| | methoxybenzyl)amino]methyl}benzenesulfonamide |
| 960. | N-acetyl-4-({butyl[(1-butyl-2,4-diphenyl-1H-imidazol-5- |
| | yl)methyl]amino}methyl)benzenesulfonamide |
| 961. | 4-(5-{[(1,3-benzodioxol-5-ylmethyl)(butyl)amino]methyl}-1-butyl-2-phenyl-1H- |
| | imidazol-4-yl)benzonitrile |
| 962. | N-[4-(5-{[(1,3-benzodioxol-5-ylmethyl)(butyl)amino]methyl}-1-butyl-2-phenyl-1H- |
| | imidazol-4-yl)benzyl]methanesulfonamide |
| 963. | N′-[4-(5-{[(1,3-benzodioxol-5-ylmethyl)(butyl)amino]methyl}-1-butyl-2-phenyl-1H- |
| | imidazol-4-yl)benzyl]-N,N-dimethylsulfamide |
| 964. | N-(1,3-benzodioxol-5-ylmethyl)-N-[(1-butyl-4-{4-[(dimethylamino)methyl]phenyl}-2- |
| | phenyl-1H-imidazol-5-yl)methyl]butan-1-amine |
| 965. | methyl 4-(5-{[(1,3-benzodioxol-5-ylmethyl)(butyl)amino]methyl}-1-butyl-2-phenyl- |
| | 1H-imidazol-4-yl)benzylcarbamate |
| 966. | 4-[(butyl{[1-butyl-4-(4-cyanophenyl)-2-phenyl-1H-imidazol-5- |
| | yl]methyl}amino)methyl]phenyl acetate |
| 967. | 4-[(butyl{[1-butyl-2-(3-methoxyphenyl)-4-phenyl-1H-imidazol-5- |
| | yl]methyl}amino)methyl]-3-chlorophenol |
| 968. | N-(1,3-benzodioxol-5-ylmethyl)-N-{[1-butyl-2-(3-methoxyphenyl)-4-phenyl-1H- |
| | imidazol-5-yl]methyl}butan-1-amine |
| 969. | 4-[(butyl{[1-butyl-2-(3-methoxyphenyl)-4-phenyl-1H-imidazol-5- |
| | yl]methyl}amino)methyl]benzoic acid |
| 970. | N-{[1 -butyl-2-(3-methoxyphenyl)-4-phenyl-1H-imidazol-5-yl]methyl}-N-(4- |
| | methoxybenzyl)butan-1-amine |
| 971. | 4-[(benzyl{[1-butyl-2-(3-methoxyphenyl)-4-phenyl-1H-imidazol-5- |
| | yl]methyl}amino)methyl]-3-chlorophenol |
| 972. | 4-{[[(1-butyl-2,4-diphenyl-1H-imidazol-5-yl)methyl](4- |
| | hydroxybenzyl)amino]methyl}benzenesulfonamide |
| 973. | N-acetyl-4-({(1,3-benzodioxol-5-ylmethyl)[(1-butyl-2,4-diphenyl-1H-imidazol-5- |
| | yl)methyl]amino}methyl)benzenesulfonamide |
| 974. | N-acetyl-4-({benzyl[(1-butyl-2,4-diphenyl-1H-imidazol-5- |
| | yl)methyl]amino}methyl)benzenesulfonamide |
| 975. | N-[4-({(1,3-benzodioxol-5-ylmethyl)[(1-butyl-2,4-diphenyl-1H-imidazol-5- |
| | yl)methyl]amino}methyl)benzoyl]methanesulfonamide |
| 976. | N-[4-({benzyl[(1-butyl-2,4-diphenyl-1H-imidazol-5-yl)methyl]amino}methyl)phenyl]- |
| | N'-ethylurea |
| 977. | N-[4-({butyl[(1-butyl-2,4-diphenyl-1H-imidazol-5-yl)methyl]amino}methyl)-2- |
| | hydroxybenzyl]methanesulfonamide |
| 978. | N-(4-{[[(1-butyl-2,4-diphenyl-1H-imidazol-5- |
| | yl)methyl](cyclohexylmethyl)amino]methyl}-2-hydroxybenzyl)methanesulfonamide |
| 979. | 4-({(1,3-benzodioxol-5-ylmethyl)[1-(1-butyl-2,4-diphenyl-1H-imidazol-5- |
| | yl)ethyl]amino}methyl)benzoic acid |
| 980. | 4-({[4-(aminosulfonyl)benzyl][(1-butyl-2,4-diphenyl-1H-imidazol-5- |
| | yl)methyl]amino}methyl)benzoic acid |
| 981. | N-[4-({butyl[(1-butyl-2,4-diphenyl-1H-imidazol-5-yl)methyl]amino}methyl)-2- |
| | methoxybenzyl]methanesulfonamide |
| 982. | N-(4-{[[(1-butyl-2,4-diphenyl-1H-imidazol-5- |
| | yl)methyl](cyclohexylmethyl)amino]methyl}-2-methoxybenzyl)methanesulfonamide |
| 983. | 1-(1,3-benzodioxol-5-yl)-N-benzyl-N-{[1-butyl-2-(3-methoxyphenyl)-4-phenyl-1H- |
| | imidazol-5-yl]methyl}methanamine |
| 984. | 4-[(benzyl{[1-butyl-2-(3-methoxyphenyl)-4-phenyl-1H-imidazol-5- |
| | yl]methyl}amino)methyl]benzoic acid |
| 985. | N-(1,3-benzodioxol-5-ylmethyl)-N-[(1-butyl-2,4-diphenyl-1H-imidazol-5-yl)methyl]-4- |
| | methylbenzenesulfonamide |
| 986. | 4-({[4-(aminosulfonyl)benzyl][(1-butyl-2,4-diphenyl-1H-imidazol-5- |
| | yl)methyl]amino}methyl)benzamide |
| 987. | methyl 4-{[[(1-butyl-2,4-diphenyl-1H-imidazol-5-yl)methyl](4- |
| | hydroxybenzyl)amino]methyl}benzoate |
| 988. | methyl 4-{[[(1-butyl-2,4-diphenyl-1H-imidazol-5-yl)methyl](2-chloro-4- |
| | hydroxybenzyl)amino]methyl}benzoate |
| 989. | methyl 4-{[[(1-butyl-2,4-diphenyl-1H-imidazol-5-yl)methyl](4- |
| | methoxybenzyl)amino]methyl}benzoate |
| 990. | diethyl 5-({benzyl[(1-butyl-2,4-diphenyl-1H-imidazol-5- |
| | yl)methyl]amino}methyl)isophthalate |
| 991. | diethyl 5-({butyl[(1-butyl-2,4-diphenyl-1H-imidazol-5- |
| | yl)methyl]amino}methyl)isophthalate |
| 992. | 4-{[[(1-butyl-2,4-diphenyl-1H-imidazol-5-yl)methyl](4- |
| | hydroxybenzyl)amino]methyl}benzoic acid |
| 993. | 4-{[[(1-butyl-2,4-diphenyl-1H-imidazol-5-yl)methyl](2-chloro-4- |
| | hydroxybenzyl)amino]methyl}benzoic acid |
| 994. | 4-{[[(1-butyl-2,4-diphenyl-1H-imidazol-5-yl)methyl](4- |
| | methoxybenzyl)amino]methyl}benzoic acid |
| 995. | 5-({benzyl[(1-butyl-2,4-diphenyl-1H-imidazol-5-yl)methyl]amino}methyl)isophthalic |
| | acid |
| 996. | N-[4-({butyl[(1-butyl-2,4-diphenyl-1H-imidazol-5- |
| | yl)methyl]amino}methyl)benzyl]methanesulfonamide |
| 997. | 4-({[(1-butyl-2,4-diphenyl-1H-imidazol-5-yl)methyl][4- |
| | (hydroxymethyl)benzyl]amino}methyl)phenol |
| 998. | 4-({[(1-butyl-2,4-diphenyl-1H-imidazol-5-yl)methyl][4- |
| | (hydroxymethyl)benzyl]amino}methyl)-3-chlorophenol |
| 999. | (4-{[[(1-butyl-2,4-diphenyl-1H-imidazol-5-yl)methyl](4- |
| | methoxybenzyl)amino]methyl}phenyl)methanol |
| 1000. | N-benzyl-1-(1-butyl-2,4-diphenyl-1H-imidazol-5-yl)-N-(2,4- |
| | dihydroxymethylbenzyl)methanamine |
| 1001. | 1-(1-butyl-2,4-diphenyl-1H-imidazol-5-yl)-N-cyclohexylmethyl-N-(2,4- |
| | dihydroxymethylbenzyl)methanamine |
| 1002. | 4-({(1,3 -benzodioxol-5-ylmethyl)[(2,4-diphenyl-1-propyl-1H-imidazol-5- |
| | yl)methyl]amino}methyl)benzenesulfonamide |
| 1003. | methyl 4-[((1,3-benzodioxol-5-ylmethyl){[1-(4-ethoxy-4-oxobutyl)-2,4-diphenyl-1H- |
| | imidazol-5-yl]methyl}amino)methyl]benzoate |
| 1004. | methyl 4-[((2-chloro-4-hydroxybenzyl){[1-(4-ethoxy-4-oxobutyl)-2,4-diphenyl-1H- |
| | imidazol-5-yl]methyl}amino)methyl]benzoate |
| 1005. | methyl 4-{[{[1-(4-ethoxy-4-oxobutyl)-2,4-diphenyl-1H-imidazol-5-yl]methyl}(4- |
| | methoxybenzyl)amino]methyl}benzoate |
| 1006. | methyl 4-[([4-(dimethylamino)benzyl]{[1-(4-ethoxy-4-oxobutyl)-2,4-diphenyl-1H- |
| | imidazol-5-yl]methyl}amino)methyl]benzoate |
| 1007. | ethyl 4-(5-{[bis(1,3-benzodioxol-5-ylmethyl)amino]methyl}-2,4-diphenyl-1H-imidazol- |
| | 1-yl)butanoate |
| 1008. | ethyl 4-(5-{[(1,3-benzodioxol-5-ylmethyl)(2-chloro-4-hydroxybenzyl)amino]methyl}- |
| | 2,4-diphenyl-1H-imidazol-1-yl)butanoate |
| 1009. | ethyl 4-(5-{[(1,3-benzodioxol-5-ylmethyl)(4-methoxybenzyl)amino]methyl}-2,4- |
| | diphenyl-1H-imidazol-1-yl)butanoate |
| 1010. | ethyl 4-[5-({(1,3-benzodioxol-5-ylmethyl)[4-(dimethylamino)benzyl]amino}methyl)- |
| | 2,4-diphenyl-1H-imidazol-1-yl]butanoate |
| 1011. | methyl 4-({[(1-butyl-2,4-diphenyl-1H-imidazol-5-yl)methyl][4- |
| | (trifluoromethoxy)benzyl]amino}methyl)benzoate |
| 1012. | methyl 4-{[[(1-butyl-2,4-diphenyl-1H-imidazol-5-yl)methyl](3- |
| | methoxybenzyl)amino]methyl}benzoate |
| 1013. | methyl 4-({[(1-butyl-2,4-diphenyl-1H-imidazol-5-yl)methyl][4- |
| | (dimethylamino)benzyl]amino}methyl)benzoate |
| 1014. | 4-[5-({(1,3-benzodioxol-5-ylmethyl)[4-(hydroxymethyl)benzyl]amino}methyl)-2,4- |
| | diphenyl-1H-imidazol-1-yl]butan-1-ol |
| 1015. | 4-[5-({[4-(dimethylamino)benzyl][4-(hydroxymethyl)benzyl]amino}methyl)-2,4- |
| | diphenyl-1H-imidazol-1-yl]butan-1-ol |
| 1016. | 4-(5-{[bis(1,3-benzodioxol-5-ylmethyl)amino]methyl}-2,4-diphenyl-1H-imidazol-1- |
| | yl)butan-1-ol |
| 1017. | 4-[((1,3-benzodioxol-5-ylmethyl){[1-(4-hydroxybutyl)-2,4-diphenyl-1H-imidazol-5- |
| | yl]methyl}amino)methyl]-3-chlorophenol |
| 1018. | 4-(5-{[(1,3-benzodioxol-5-ylmethyl)(4-methoxybenzyl)amino]methyl}-2,4-diphenyl- |
| | 1H-imidazol-1-yl)butan-1-ol |
| 1019. | 4-[5-({(1,3-benzodioxol-5-ylmethyl)[4-(dimethylamino)benzyl]amino}methyl)-2,4- |
| | diphenyl-1H-imidazol-1-yl]butan-1-ol |
| 1020. | [4-({[(1-butyl-2,4-diphenyl-1H-imidazol-5-yl)methyl][4- |
| | (trifluoromethoxy)benzyl]amino}methyl)phenyl]methanol |
| 1021. | (4-{[[(1-butyl-2,4-diphenyl-1H-imidazol-5-yl)methyl](3- |
| | methoxybenzyl)amino]methyl}phenyl)methanol |
| 1022. | [4-({[(1-butyl-2,4-diphenyl-1H-imidazol-5-yl)methyl][4- |
| | (dimethylamino)benzyl]amino}methyl)phenyl]methanol |
| 1023. | 4-(5-{[[4-(hydroxymethyl)benzyl](4-methoxybenzyl)amino]methyl}-2,4-diphenyl-1H- |
| | imidazol-1-yl)butan-1-ol |
| 1024. | 4-(5-{[bis(1,3-benzodioxol-5-ylmethyl)amino]methyl}-2,4-diphenyl-1H-imidazol-1- |
| | yl)butanoic acid |
| 1025. | 4-(5-{[(1,3-benzodioxol-5-ylmethyl)(2-chloro-4-hydroxybenzyl)amino]methyl}-2,4- |
| | diphenyl-1H-imidazol-1-yl)butanoic acid |
| 1026. | 4-[(benzyl{[1-butyl-2-(2-methoxyphenyl)-4-phenyl-1H-imidazol-5- |
| | yl]methyl}amino)methyl]-3-chlorophenol |
| 1027. | 1-(1,3-benzodioxol-5-yl)-N-benzyl-N-{[1-butyl-2-(2-methoxyphenyl)-4-phenyl-1H- |
| | imidazol-5-yl]methyl}methanamine |
| 1028. | 4-[(benzyl{[1-butyl-2-(2-methoxyphenyl)-4-phenyl-1H-imidazol-5- |
| | yl]methyl}amino)methyl]benzenesulfonamide |
| 1029. | 4-[(butyl{[1-butyl-2-(2-methoxyphenyl)-4-phenyl-1H-imidazol-5- |
| | yl]methyl}amino)methyl]-3-chlorophenol |
| 1030. | N-(1,3-benzodioxol-5-ylmethyl)-N-{[1-butyl-2-(2-methoxyphenyl)-4-phenyl-1H- |
| | imidazol-5-yl]methyl}butan-1-amine |
| 1031. | 4-[(butyl{[1-butyl-2-(2-methoxyphenyl)-4-phenyl-1H-imidazol-5- |
| | yl]methyl}amino)methyl]phenol |
| 1032. | 4-[(butyl{[1-butyl-4-(4-methoxyphenyl)-2-phenyl-1H-imidazol-5- |
| | yl]methyl}amino)methyl]benzoic acid |
| 1033. | 4-[(butyl{[1-butyl-2-(3-methoxyphenyl)-4-phenyl-1H-imidazol-5- |
| | yl]methyl}amino)methyl]benzamide |
| 1034. | 4-[(butyl{[1-butyl-2-(2-methoxyphenyl)-4-phenyl-1H-imidazol-5- |
| | yl]methyl}amino)methyl]benzenesulfonamide |
| 1035. | 4-[(butyl{[1-butyl-2-(3-methoxyphenyl)-4-phenyl-1H-imidazol-5- |
| | yl]methyl}amino)methyl]benzenesulfonamide |
| 1036. | 4-[(butyl{[1-butyl-4-(4-methoxyphenyl)-2-phenyl-1H-imidazol-5- |
| | yl]methyl}amino)methyl]benzenesulfonamide |
| 1037. | 4-({[1-butyl-2,4-diphenyl-1H-imidazol-5-yl)methyl][4- |
| | (trifluoromethoxy)benzyl]amino}methyl)benzoic acid |
| 1038. | 4-{[[(1-butyl-2,4-diphenyl-1H-imidazol-5-yl)methyl](3- |
| | methoxybenzyl)amino]methyl}benzoic acid |
| 1039. | 4-({[(1-butyl-2,4-diphenyl-1H-imidazol-5-yl)methyl][4- |
| | (dimethylamino)benzyl]amino}methyl)benzoic acid |
| 1040. | 4-[((1,3-benzodioxol-5-ylmethyl){[1-butyl-4-(4-methoxyphenyl)-2-phenyl-1H- |
| | imidazol-5-yl]methyl}amino)methyl]benzoic acid |
| 1041. | 1-[1-butyl-4-(4-methoxyphenyl)-2-phenyl-1H-imidazol-5-yl]-N,N-bis(4- |
| | methoxybenzyl)methanamine |
| 1042. | methyl 3-({butyl[(1-butyl-2,4-diphenyl-1H-imidazol-5-yl)methyl]amino}methyl)-5- |
| | methoxybenzoate |
| 1043. | methyl 3-({[(1-butyl-2,4-diphenyl-1H-imidazol-5-yl)methyl][4- |
| | (methoxycarbonyl)benzyl]amino}methyl)-5-methoxybenzoate |
| 1044. | 4-({butyl[1-(1-butyl-2,4-diphenyl-1H-imidazol-5-yl)ethyl]amino}methyl)benzamide |
| 1045. | 4-{[[(1-butyl-2,4-diphenyl-1H-imidazol-5- |
| | yl)methyl](cyclohexylmethyl)amino]methyl}-2,6-dimethylphenol |
| 1046. | 4-{[[(1-butyl-2,4-diphenyl-1H-imidazol-5- |
| | yl)methyl](cyclohexylmethyl)amino]methyl}-2-methylphenol |
| 1047. | 4-({butyl[1-(1-butyl-2,4-diphenyl-1H-imidazol-5-yl)ethyl]amino}methyl)-2,6- |
| | dimethylphenol |
| 1048. | 4-({butyl[(1-butyl-2,4-diphenyl-1H-imidazol-5-yl)methyl]amino}methyl)-2,6- |
| | dimethylphenol |
| 1049. | [3-({butyl[(1-butyl-2,4-diphenyl-1H-imidazol-5-yl)methyl]amino}methyl)-5- |
| | methoxyphenyl]methanol |
| 1050. | [3-({[(1-butyl-2,4-diphenyl-1H-imidazol-5-yl)methyl][4- |
| | (hydroxymethyl)benzyl]amino}methyl)-5-methoxyphenyl]methanol |
| 1051. | 4-({benzyl[(1-butyl-2,4-diphenyl-1H-imidazol-5-yl)methyl]amino}methyl)-2- |
| | nitrophenol |
| 1052. | N-benzyl-N-[(1-butyl-2,4-diphenyl-1H-imidazol-5-yl)methyl]butan-1- |
| | amine |
| 1053. | N,N-dibenzyl-1-(1-butyl-2,4-diphenyl-1H-imidazol-5-yl)methanamine |
| 1054. | N-benzyl-1-(1-butyl-2,4-diphenyl-1H-imidazol-5-yl)-N-(4- |
| | methoxybenzyl)methanamine |
| 1055. | N-benzyl-1-(1-butyl-2,4-diphenyl-1H-imidazol-5-yl)-N- |
| | (cyclohexylmethyl)methanamine |
| 1056. | N-(2-bromobenzyl)-N-[(1-butyl-2,4-diphenyl-1H-imidazol-5-yl)methyl]butan-1-amine |
| 1057. | N-benzyl-1-(2-bromophenyl)-N-[(1-butyl-2,4-diphenyl-1H-imidazol-5- |
| | yl)methyl]methanamine |
| 1058. | 1-(2-bromophenyl)-N-[(1-butyl-2,4-diphenyl-1H-imidazol-5-yl)methyl]-N-(4- |
| | methoxybenzyl)methanamine |
| 1059. | 1-(2-bromophenyl)-N-[(1-butyl-2,4-diphenyl-1H-imidazol-5-yl)methyl]-N- |
| | (cyclohexylmethyl)methanamine |
| 1060. | N-[(1-butyl-2,4-diphenyl-1H-imidazol-5-yl)methyl]-N-(2-fluorobenzyl)butan-1-amine |
| 1061. | N-benzyl-1-(1-butyl-2,4-diphenyl-1H-imidazol-5-yl)-N-(2-fluorobenzyl)methanamine |
| 1062. | 1-(1-butyl-2,4-diphenyl-1H-imidazol-5-yl)-N-(2-fluorobenzyl)-N-(4- |
| | methoxybenzyl)methanamine |
| 1063. | 1-(1-butyl-2,4-diphenyl-1H-imidazol-5-yl)-N-(cyclohexylmethyl)-N-(2- |
| | fluorobenzyl)methanamine |
| 1064. | N-[(1-butyl-2,4-diphenyl-1H-imidazol-5-yl)methyl]-N-(2-chlorobenzyl)butan-1-amine |
| 1065. | N-benzyl-1-(1-butyl-2,4-diphenyl-1H-imidazol-5-yl)-N-(2-chlorobenzyl)methanamine |
| 1066. | 1-(1-butyl-2,4-diphenyl-1H-imidazol-5-yl)-N-(2-chlorobenzyl)-N-(4- |
| | methoxybenzyl)methanamine |
| 1067. | 1-(1-butyl-2,4-diphenyl-1H-imidazol-5-yl)-N-(2-chlorobenzyl)-N- |
| | (cyclohexylmethyl)methanamine |
| 1068. | N-[(1-butyl-2,4-diphenyl-1H-imidazol-5-yl)methyl]-N-(2-methylbenzyl)butan-1-amine |
| 1069. | N-benzyl-1-(1-butyl-2,4-diphenyl-1H-imidazol-5-yl)-N-(2-methylbenzyl)methanamine |
| 1070. | 1-(1-butyl-2,4-diphenyl-1H-imidazol-5-yl)-N-(4-methoxybenzyl)-N-(2- |
| | methylbenzyl)methanamine |
| 1071. | 1-(1-butyl-2,4-diphenyl-1H-imidazol-5-yl)-N-(cyclohexylmethyl)-N-(2- |
| | methylbenzyl)methanamine |
| 1072. | N-[(1-butyl-2,4-diphenyl-1H-imidazol-5-yl)methyl]-N-(2-methoxybenzyl)butan-1- |
| | amine |
| 1073. | N-benzyl-1-(1-butyl-2,4-diphenyl-1H-imidazol-5-yl)-N-(2- |
| | methoxybenzyl)methanamine |
| 1074. | 1-(1-butyl-2,4-diphenyl-1H-imidazol-5-yl)-N-(2-methoxybenzyl)-N-(4- |
| | methoxybenzyl)methanamine |
| 1075. | 1-(1-butyl-2,4-diphenyl-1H-imidazol-5-yl)-N-(cyclohexylmethyl)-N-(2- |
| | methoxybenzyl)methanamine |
| 1076. | N-[(1-butyl-2,4-diphenyl-1H-imidazol-5-yl)methyl]-N-[2- |
| | (trifluoromethyl)benzyl]butan-1-amine |
| 1077. | N-benzyl-1-(1-butyl-2,4-diphenyl-1H-imidazol-5-yl)-N-[2- |
| | (trifluoromethyl)benzyl]methanamine |
| 1078. | 1-(1-butyl-2,4-diphenyl-1H-imidazol-5-yl)-N-(4-methoxybenzyl)-N-[2- |
| | (trifluoromethyl)benzyl]methanamine |
| 1079. | 1-(1-butyl-2,4-diphenyl-1H-imidazol-5-yl)-N-(cyclohexylmethyl)-N-[2- |
| | (trifluoromethyl)benzyl]methanamine |
| 1080. | N-[(1-butyl-2,4-diphenyl-1H-imidazol-5-yl)methyl]-N-(2-ethoxybenzyl)butan-1-amine |
| 1081. | N-benzyl-1-(1-butyl-2,4-diphenyl-1H-imidazol-5-yl)-N-(2-ethoxybenzyl)methanamine |
| 1082. | 1-(1-butyl-2,4-diphenyl-1H-imidazol-5-yl)-N-(2-ethoxybenzyl)-N-(4- |
| | methoxybenzyl)methanamine |
| 1083. | 1-(1-butyl-2,4-diphenyl-1H-imidazol-5-yl)-N-(cyclohexylmethyl)-N-(2- |
| | ethoxybenzyl)methanamine |
| 1084. | N-[(1-butyl-2,4-diphenyl-1H-imidazol-5-yl)methyl]-N-(2,4-dichlorobenzyl)butan-1- |
| | amine |
| 1085. | N-benzyl-1-(1-butyl-2,4-diphenyl-1H-imidazol-5-yl)-N-(2,4- |
| | dichlorobenzyl)methanamine |
| 1086. | 1-(1-butyl-2,4-diphenyl-1H-imidazol-5-yl)-N-(cyclohexylmethyl)-N-(2,4- |
| | dichlorobenzyl)methanamine |
| 1087. | N-[(1-butyl-2,4-diphenyl-1H-imidazol-5-yl)methyl]-N-(2,4-dimethoxybenzyl)butan-1- |
| | amine |
| 1088. | N-benzyl-1-(1-butyl-2,4-diphenyl-1H-imidazol-5-yl)-N-(2,4- |
| | dimethoxybenzyl)methanamine |
| 1089. | 1-(1-butyl-2,4-diphenyl-1H-imidazol-5-yl)-N-(2,4-dimethoxybenzyl)-N-(4- |
| | methoxybenzyl)methanamine |
| 1090. | 1-(1-butyl-2,4-diphenyl-1H-imidazol-5-yl)-N-(cyclohexylmethyl)-N-(2,4- |
| | dimethoxybenzyl)methanamine |
| 1091. | N-[(1-butyl-2,4-diphenyl-1H-imidazol-5-yl)methyl]-N-(2,5-dimethoxybenzyl)butan-1- |
| | amine |
| 1092. | N-benzyl-1-(1-butyl-2,4-diphenyl-1H-imidazol-5-yl)-N-(2,5- |
| | dimethoxybenzyl)methanamine |
| 1093. | 1-(1-butyl-2,4-diphenyl-1H-imidazol-5-yl)-N-(2,5-dimethoxybenzyl)-N-(4- |
| | methoxybenzyl)methanamine |
| 1094. | 1-(1-butyl-2,4-diphenyl-1H-imidazol-5-yl)-N-(cyclohexylmethyl)-N-(2,5- |
| | dimethoxybenzyl)methanamine |
| 1095. | N-(4-bromobenzyl)-N-[(1-butyl-2,4-diphenyl-1H-imidazol-5-yl)methyl]butan-1-amine |
| 1096. | N-benzyl-1-(4-bromophenyl)-N-[(1-butyl-2,4-diphenyl-1H-imidazol-5- |
| | yl)methyl]methanamine |
| 1097. | 1-(4-bromophenyl)-N-[(1-butyl-2,4-diphenyl-1H-imidazol-5-yl)methyl]-N-(4- |
| | methoxybenzyl)methanamine |
| 1098. | 1-(4-bromophenyl)-N-[(1-butyl-2,4-diphenyl-1H-imidazol-5-yl)methyl]-N- |
| | (cyclohexylmethyl)methanamine |
| 1099. | N-[(1-butyl-2,4-diphenyl-1H-imidazol-5-yl)methyl]-N-(4-fluorobenzyl)butan-1-amine |
| 1100. | N-benzyl-1-(1-butyl-2,4-diphenyl-1H-imidazol-5-yl)-N-(4-fluorobenzyl)methanamine |
| 1101. | 1-(1-butyl-2,4-diphenyl-1H-imidazol-5-yl)-N-(cyclohexylmethyl)-N-(4- |
| | fluorobenzyl)methanamine |
| 1102. | N-[(1-butyl-2,4-diphenyl-1H-imidazol-5-yl)methyl]-N-(4-chlorobenzyl)butan-1-amine |
| 1103. | N-benzyl-1-(1-butyl-2,4-diphenyl-1H-imidazol-5-yl)-N-(4-chlorobenzyl)methanamine |
| 1104. | 1-(1-butyl-2,4-diphenyl-1H-imidazol-5-yl)-N-(4-chlorobenzyl)-N-(4- |
| | methoxybenzyl)methanamine |
| 1105. | 1-(1-butyl-2,4-diphenyl-1H-imidazol-5-yl)-N-(4-chlorobenzyl)-N- |
| | (cyclohexylmethyl)methanamine |
| 1106. | N-[(1-butyl-2,4-diphenyl-1H-imidazol-5-yl)methyl]-N-(4-methylbenzyl)butan-1-amine |
| 1107. | N-benzyl-1-(1-butyl-2,4-diphenyl-1H-imidazol-5-yl)-N-(4-methylbenzyl)methanamine |
| 1108. | 1-(1-butyl-2,4-diphenyl-1H-imidazol-5-yl)-N-(4-methoxybenzyl)-N-(4- |
| | methylbenzyl)methanamine |
| 1109. | 1-(1-butyl-2,4-diphenyl-1H-imidazol-5-yl)-N-(cyclohexylmethyl)-N-(4- |
| | methylbenzyl)methanamine |
| 1110. | N-[(1-butyl-2,4-diphenyl-1H-imidazol-5-yl)methyl]-N-(4-ethylbenzyl)butan-1-amine |
| 1111. | N-benzyl-1-(1-butyl-2,4-diphenyl-1H-imidazol-5-yl)-N-(4-ethylbenzyl)methanamine |
| 1112. | 1-(1-butyl-2,4-diphenyl-1H-imidazol-5-yl)-N-(4-ethylbenzyl)-N-(4- |
| | methoxybenzyl)methanamine |
| 1113. | 1-(1-butyl-2,4-diphenyl-1H-imidazol-5-yl)-N-(cyclohexylmethyl)-N-(4 |
| | ethylbenzyl)methanamine |
| 1114. | N-[(1-butyl-2,4-diphenyl-1H-imidazol-5-yl)methyl]-N-(4-methoxybenzyl)butan-1- |
| | amine |
| 1115. | 1-(1-butyl-2,4-diphenyl-1H-imidazol-5-yl)-N,N-bis(4-methoxybenzyl)methanamine |
| 1116. | 1-(1-butyl-2,4-diphenyl-1H-imidazol-5-yl)-N-(cyclohexylmethyl)-N-(4- |
| | methoxybenzyl)methanamine |
| 1117. | N-[(1-butyl-2,4-diphenyl-1H-imidazol-5-yl)methyl]-N-(4-ethoxybenzyl)butan-1-amine |
| 1118. | N-benzyl-1-(1-butyl-2,4-diphenyl-1H-imidazol-5-yl)-N-(4-ethoxybenzyl)methanamine |
| 1119. | 1-(1-butyl-2,4-diphenyl-1H-imidazol-5-yl)-N-(4-ethoxybenzyl)-N-(4- |
| | methoxybenzyl)methanamine |
| 1120. | 1-(1-butyl-2,4-diphenyl-1H-imidazol-5-yl)-N-(cyclohexylmethyl)-N-(4- |
| | ethoxybenzyl)methanamine |
| 1121. | N-(4-butoxybenzyl)-N-[(1-butyl-2,4-diphenyl-1H-imidazol-5-yl)methyl]butan-1-amine |
| 1122. | N-benzyl-1-(4-butoxyphenyl)-N-[(1-butyl-2,4-diphenyl-1H-imidazol-5- |
| | yl)methyl]methanamine |
| 1123. | 1-(4-butoxyphenyl)-N-[(1-butyl-2,4-diphenyl-1H-imidazol-5-yl)methyl]-N-(4- |
| | methoxybenzyl)methanamine |
| 1124. | 1-(4-butoxyphenyl)-N-[(1-butyl-2,4-diphenyl-1H-imidazol-5-yl)methyl]-N- |
| | (cyclohexylmethyl)methanamine |
| 1125. | N-[(1-butyl-2,4-diphenyl-1H-imidazol-5-yl)methyl]-N-[4- |
| | (trifluoromethyl)benzyl]butan-1-amine |
| 1126. | N-benzyl-1-(1-butyl-2,4-diphenyl-1H-imidazol-5-yl)-N-[4- |
| | (trifluoromethyl)benzyl]methanamine |
| 1127. | 1-(1-butyl-2,4-diphenyl-1H-imidazol-5-yl)-N-(4-methoxybenzyl)-N-[4- |
| | (trifluoromethyl)benzyl]methanamine |
| 1128. | 1-(1-butyl-2,4-diphenyl-1H-imidazol-5-yl)-N-(cyclohexylmethyl)-N-[4- |
| | (trifluoromethyl)benzyl]methanamine |
| 1129. | N-[(1-butyl-2,4-diphenyl-1H-imidazol-5-yl)methyl]-N-(4-isopropylbenzyl)butan-1- |
| | amine |
| 1130. | N-benzyl-1-(1-butyl-2,4-diphenyl-1H-imidazol-5-yl)-N-(4- |
| | isopropylbenzyl)methanamine |
| 1131. | 1-(1-butyl-2,4-diphenyl-1H-imidazol-5-yl)-N-(4-isopropylbenzyl)-N-(4- |
| | methoxybenzyl)methanamine |
| 1132. | 1-(1-butyl-2,4-diphenyl-1H-imidazol-5-yl)-N-(cyclohexylmethyl)-N-(4- |
| | isopropylbenzyl)methanamine |
| 1133. | N-(1,1′-biphenyl-4-ylmethyl)-N-[(1-butyl-2,4-diphenyl-1H-imidazol-5-yl)methyl]butan- |
| | 1-amine |
| 1134. | N-benzyl-1-(1,1′-biphenyl-4-yl)-N-[(1-butyl-2,4-diphenyl-1H-imidazol-5- |
| | yl)methyl]methanamine |
| 1135. | N-(1,1′-biphenyl-4-ylmethyl)-N-[(1-butyl-2,4-diphenyl-1H-imidazol-5-yl)methyl]-N-(4- |
| | methoxybenzyl)amine |
| 1136. | N-(1,1′-biphenyl-4-ylmethyl)-N-[(1-butyl-2,4-diphenyl-1H-imidazol-5-yl)methyl]-N- |
| | (cyclohexylmethyl)amine |
| 1137. | N-(1,3-benzodioxol-5-ylmethyl)-N-[(1-butyl-2,4-diphenyl-1H-imidazol-5- |
| | yl)methyl]butan-1-amine |
| 1138. | 1-(1,3-benzodioxol-5-yl)-N-benzyl-N-[(1-butyl-2,4-diphenyl-1H-imidazol-5- |
| | yl)methyl]methanamine |
| 1139. | 1-(1,3-benzodioxol-5-yl)-N-[(1-butyl-2,4-diphenyl-1H-imidazol-5-yl)methyl]-N-(4- |
| | methoxybenzyl)methanamine |
| 1140. | 1-(1,3-benzodioxol-5-yl)-N-[(1-butyl-2,4-diphenyl-1H-imidazol-5-yl)methyl]-N- |
| | (cyclohexylmethyl)methanamine |
| 1141. | N-[(1-butyl-2,4-diphenyl-1H-imidazol-5-yl)methyl]-N-(2,3-dihydro-1,4-benzodioxin-6- |
| | ylmethyl)butan-1-amine |
| 1142. | N-benzyl-1-(1-butyl-2,4-diphenyl-1H-imidazol-5-yl)-N-(2,3-dihydro-1,4-benzodioxin- |
| | 6-ylmethyl)methanamine |
| 1143. | 1-(1-butyl-2,4-diphenyl-1H-imidazol-5-yl)-N-(2,3-dihydro-1,4-benzodioxin-6- |
| | ylmethyl)-N-(4-methoxybenzyl)methanamine |
| 1144. | 1-(1-butyl-2,4-diphenyl-1H-imidazol-5-yl)-N-(cyclohexylmethyl)-N-(2,3-dihydro-1,4- |
| | benzodioxin-6-ylmethyl)methanamine |
| 1145. | N-[(1-butyl-2,4-diphenyl-1H-imidazol-5-yl)methyl]-N-(4-phenoxybenzyl)butan-1- |
| | amine |
| 1146. | 1-(1-butyl-2,4-diphenyl-1H-imidazol-5-yl)-N-(cyclohexylmethyl)-N-(4- |
| | phenoxybenzyl)methanamine |
| 1147. | N-[(1-butyl-2,4-diphenyl-1H-imidazol-5-yl)methyl]-N-(2,4,6-trimethoxybenzyl)butan- |
| | 1-amine |
| 1148. | N-benzyl-1-(1-butyl-2,4-diphenyl-1H-imidazol-5-yl)-N-(2,4,6- |
| | trimethoxybenzyl)methanamine |
| 1149. | 1-(1-butyl-2,4-diphenyl-1H-imidazol-5-yl)-N-(4-methoxybenzyl)-N-(2,4,6- |
| | trimethoxybenzyl)methanamine |
| 1150. | 1-(1-butyl-2,4-diphenyl-1H-imidazol-5-yl)-N-(cyclohexylmethyl)-N-(2,4,6- |
| | trimethoxybenzyl)methanamine |
| 1151. | N-[3-(benzyloxy)benzyl]-N-[(1-butyl-2,4-diphenyl-1H-imidazol-5-yl)methyl]-N- |
| | (cyclohexylmethyl)amine |
| 1152. | N-[4-(benzyloxy)benzyl]-N-[(1-butyl-2,4-diphenyl-1H-imidazol-5-yl)methyl]butan-1- |
| | amine |
| 1153. | N-[4-(benzyloxy)benzyl]-N-[(1-butyl-2,4-diphenyl-1H-imidazol-5-yl)methyl]-N-(4- |
| | methoxybenzyl)amine |
| 1154. | N-[4-(benzyloxy)benzyl]-N-[(1-butyl-2,4-diphenyl-1H-imidazol-5-yl)methyl]-N- |
| | (cyclohexylmethyl)amine |
| 1155. | N-[3-(benzyloxy)-4-methoxybenzyl]-N-[(1-butyl-2,4-diphenyl-1H-imidazol-5- |
| | yl)methyl]butan-1-amine |
| 1156. | N-benzyl-1-[3-(benzyloxy)-4-methoxyphenyl]-N-[(1-butyl-2,4-diphenyl-1H-imidazol- |
| | 5-yl)methyl]methanamine |
| 1157. | 1-[3-(benzyloxy)-4-methoxyphenyl]-N-[(1-butyl-2,4-diphenyl-1H-imidazol-5- |
| | yl)methyl]-N-(cyclohexylmethyl)methanamine |
| 1158. | 1-[4-(benzyloxy)-3-methoxyphenyl]-N-[(1-butyl-2,4-diphenyl-1H-imidazol-5- |
| | yl)methyl]-N-(cyclohexylmethyl)methanamine |
| 1159. | N-[(1-butyl-2,4-diphenyl-1H-imidazol-5-yl)methyl]-N-(3,4-diethoxybenzyl)butan-1- |
| | amine |
| 1160. | N-benzyl-1-(1-butyl-2,4-diphenyl-1H-imidazol-5-yl)-N-(3,4- |
| | diethoxybenzyl)methanamine |
| 1161. | 1-(1-butyl-2,4-diphenyl-1H-imidazol-5-yl)-N-(3,4-diethoxybenzyl)-N-(4- |
| | methoxybenzyl)methanamine |
| 1162. | 1-(1-butyl-2,4-diphenyl-1H-imidazol-5-yl)-N-(cyclohexylmethyl)-N-(3,4- |
| | diethoxybenzyl)methanamine |
| 1163. | N-[(1-butyl-2,4-diphenyl-1H-imidazol-5-yl)methyl]-N-(2,3-dimethoxybenzyl)butan-1- |
| | amine |
| 1164. | N-benzyl-1-(1-butyl-2,4-diphenyl-1H-imidazol-5-yl)-N-(2,3- |
| | dimethoxybenzyl)methanamine |
| 1165. | 1-(1-butyl-2,4-diphenyl-1H-imidazol-5-yl)-N-(2,3-dimethoxybenzyl)-N-(4- |
| | methoxybenzyl)methanamine |
| 1166. | 1-(1-butyl-2,4-diphenyl-1H-imidazol-5-yl)-N-(cyclohexylmethyl)-N-(2,3- |
| | dimethoxybenzyl)methanamine |
| 1167. | N-[(1-butyl-2,4-diphenyl-1H-imidazol-5-yl)methyl]-N-(3,4-dimethoxybenzyl)butan-1- |
| | amine |
| 1168. | N-benzyl-1-(1-butyl-2,4-diphenyl-1H-imidazol-5-yl)-N-(3,4- |
| | dimethoxybenzyl)methanamine |
| 1169. | 1-(1-butyl-2,4-diphenyl-1H-imidazol-5-yl)-N-(3,4-dimethoxybenzyl)-N-(4- |
| | methoxybenzyl)methanamine |
| 1170. | 1-(1-butyl-2,4-diphenyl-1H-imidazol-5-yl)-N-(cyclohexylmethyl)-N-(3,4- |
| | dimethoxybenzyl)methanamine |
| 1171. | 1-(1-butyl-2,4-diphenyl-1H-imidazol-5-yl)-N-(4-methoxybenzyl)-N-(2- |
| | nitrobenzyl)methanamine |
| 1172. | N-[(1-butyl-2,4-diphenyl-1H-imidazol-5-yl)methyl]-N-(3-methoxy-2- |
| | nitrobenzyl)butan-1-amine |
| 1173. | 1-(1-butyl-2,4-diphenyl-1H-imidazol-5-yl)-N-(cyclohexylmethyl)-N-(3-methoxy-2- |
| | nitrobenzyl)methanamine |
| 1174. | N-[(1-butyl-2,4-diphenyl-1H-imidazol-5-yl)methyl]-N-(2-chloro-6-nitrobenzyl)butan-1- |
| | amine |
| 1175. | 1-(1-butyl-2,4-diphenyl-1H-imidazol-5-yl)-N-(2-chloro-6-nitrobenzyl)-N-(4- |
| | methoxybenzyl)methanamine |
| 1176. | 1-(1-butyl-2,4-diphenyl-1H-imidazol-5-yl)-N-(2-chloro-6-nitrobenzyl)-N- |
| | (cyclohexylmethyl)methanamine |
| 1177. | 1-(1-butyl-2,4-diphenyl-1H-imidazol-5-yl)-N-(5-chloro-2-nitrobenzyl)-N-(4- |
| | methoxybenzyl)methanamine |
| 1178. | N-[(1-butyl-2,4-diphenyl-1H-imidazol-5-yl)methyl]-N-(2,4-dimethylbenzyl)butan-1- |
| | amine |
| 1179. | N-benzyl-1-(1-butyl-2,4-diphenyl-1H-imidazol-5-yl)-N-(2,4- |
| | dimethylbenzyl)methanamine |
| 1180. | 1-(1-butyl-2,4-diphenyl-1H-imidazol-5-yl)-N-(2,4-dimethylbenzyl)-N-(4- |
| | methoxybenzyl)methanamine |
| 1181. | 1-(1-butyl-2,4-diphenyl-1H-imidazol-5-yl)-N-(cyclohexylmethyl)-N-(2,4- |
| | dimethylbenzyl)methanamine |
| 1182. | N-[(1-butyl-2,4-diphenyl-1H-imidazol-5-yl)methyl]-N-(2,5-difluorobenzyl)butan-1- |
| | amine |
| 1183. | N-benzyl-1-(1-butyl-2,4-diphenyl-1H-imidazol-5-yl)-N-(2,5- |
| | difluorobenzyl)methanamine |
| 1184. | 1-(1-butyl-2,4-diphenyl-1H-imidazol-5-yl)-N-(2,5-difluorobenzyl)-N-(4- |
| | methoxybenzyl)methanamine |
| 1185. | 1-(1-butyl-2,4-diphenyl-1H-imidazol-5-yl)-N-(cyclohexylmethyl)-N-(2,5- |
| | difluorobenzyl)methanamine |
| 1186. | N-[(1-butyl-2,4-diphenyl-1H-imidazol-5-yl)methyl]-N-(2,3-dichlorobenzyl)butan-1- |
| | amine |
| 1187. | 1-(1-butyl-2,4-diphenyl-1H-imidazol-5-yl)-N-(2,3-dichlorobenzyl)-N-(4- |
| | methoxybenzyl)methanamine |
| 1188. | 1-(1-butyl-2,4-diphenyl-1H-imidazol-5-yl)-N-(cyclohexylmethyl)-N-(2,3- |
| | dichlorobenzyl)methanamine |
| 1189. | N-[3,5-bis(trifluoromethyl)benzyl]-N-[(1-butyl-2,4-diphenyl-1H-imidazol-5- |
| | yl)methyl]butan-1-amine |
| 1190. | 1-[3,5-bis(trifluoromethyl)phenyl]-N-[(1-butyl-2,4-diphenyl-1H-imidazol-5-yl)methyl]- |
| | N-(4-methoxybenzyl)methanamine |
| 1191. | 1-[3,5-bis(trifluoromethyl)phenyl]-N-[(1-butyl-2,4-diphenyl-1H-imidazol-5-yl)methyl]- |
| | N-(cyclohexylmethyl)methanamine |
| 1192. | N-[(1-butyl-2,4-diphenyl-1H-imidazol-5-yl)methyl]-N-(3,4-difluorobenzyl)butan-1- |
| | amine |
| 1193. | N-benzyl-1-(1-butyl-2,4-diphenyl-1H-imidazol-5-yl)-N-(3,4- |
| | difluorobenzyl)methanamine |
| 1194. | 1-(1-butyl-2,4-diphenyl-1H-imidazol-5-yl)-N-(3,4-difluorobenzyl)-N-(4- |
| | methoxybenzyl)methanamine |
| 1195. | 1-(1-butyl-2,4-diphenyl-1H-imidazol-5-yl)-N-(cyclohexylmethyl)-N-(3,4- |
| | difluorobenzyl)methanamine |
| 1196. | N-[(1-butyl-2,4-diphenyl-1H-imidazol-5-yl)methyl]-N-(3,4-dichlorobenzyl)butan-1- |
| | amine |
| 1197. | 1-(1-butyl-2,4-diphenyl-1H-imidazol-5-yl)-N-(3,4-dichlorobenzyl)-N-(4- |
| | methoxybenzyl)methanamine |
| 1198. | 1-(1-butyl-2,4-diphenyl-1H-imidazol-5-yl)-N-(cyclohexylmethyl)-N-(3,4- |
| | dichlorobenzyl)methanamine |
| 1199. | N-[(1-butyl-2,4-diphenyl-1H-imidazol-5-yl)methyl]-N-(2,6-dichlorobenzyl)butan-1- |
| | amine |
| 1200. | N-benzyl-1-(1-butyl-2,4-diphenyl-1H-imidazol-5-yl)-N-(2,6- |
| | dichlorobenzyl)methanamine |
| 1201. | 1-(1-butyl-2,4-diphenyl-1H-imidazol-5-yl)-N-(2,6-dichlorobenzyl)-N-(4- |
| | methoxybenzyl)methanamine |
| 1202. | 1-(1-butyl-2,4-diphenyl-1H-imidazol-5-yl)-N-(cyclohexylmethyl)-N-(2,6- |
| | dichlorobenzyl)methanamine |
| 1203. | N-[(1-butyl-2,4-diphenyl-1H-imidazol-5-yl)methyl]-N-(2-chloro-6-fluorobenzyl)butan- |
| | 1-amine |
| 1204. | N-benzyl-1-(1-butyl-2,4-diphenyl-1H-imidazol-5-yl)-N-(2-chloro-6- |
| | fluorobenzyl)methanamine |
| 1205. | 1-(1-butyl-2,4-diphenyl-1H-imidazol-5-yl)-N-(2-chloro-6-fluorobenzyl)-N-(4- |
| | methoxybenzyl)methanamine |
| 1206. | 1-(1-butyl-2,4-diphenyl-1H-imidazol-5-yl)-N-(2-chloro-6-fluorobenzyl)-N- |
| | (cyclohexylmethyl)methanamine |
| 1207. | N-[(1-butyl-2,4-diphenyl-1H-imidazol-5-yl)methyl]-N-(2,3-difluorobenzyl)butan-1- |
| | amine |
| 1208. | N-benzyl-1-(1-butyl-2,4-diphenyl-1H-imidazol-5-yl)-N-(2,3- |
| | difluorobenzyl)methanamine |
| 1209. | 1-(1-butyl-2,4-diphenyl-1H-imidazol-5-yl)-N-(2,3-difluorobenzyl)-N-(4- |
| | methoxybenzyl)methanamine |
| 1210. | 1-(1-butyl-2,4-diphenyl-1H-imidazol-5-yl)-N-(cyclohexylmethyl)-N-(2,3- |
| | difluorobenzyl)methanamine |
| 1211. | N-[(1-butyl-2,4-diphenyl-1H-imidazol-5-yl)methyl]-N-(2,6-difluorobenzyl)butan-1- |
| | amine |
| 1212. | N-benzyl-1-(1-butyl-2,4-diphenyl-1H-imidazol-5-yl)-N-(2,6- |
| | difluorobenzyl)methanamine |
| 1213. | 1-(1-butyl-2,4-diphenyl-1H-imidazol-5-yl)-N-(2,6-difluorobenzyl)-N-(4- |
| | methoxybenzyl)methanamine |
| 1214. | 1-(1-butyl-2,4-diphenyl-1H-imidazol-5-yl)-N-(cyclohexylmethyl)-N-(2,6- |
| | difluorobenzyl)methanamine |
| 1215. | N-[(1-butyl-2,4-diphenyl-1H-imidazol-5-yl)methyl]-N-(2,4-difluorobenzyl)butan-1- |
| | amine |
| 1216. | N-benzyl-1-(1-butyl-2,4-diphenyl-1H-imidazol-5-yl)-N-(2,4- |
| | difluorobenzyl)methanamine |
| 1217. | 1-(1-butyl-2,4-diphenyl-1H-imidazol-5-yl)-N-(2,4-difluorobenzyl)-N-(4- |
| | methoxybenzyl)methanamine |
| 1218. | 1-(1-butyl-2,4-diphenyl-1H-imidazol-5-yl)-N-(cyclohexylmethyl)-N-(2,4- |
| | difluorobenzyl)methanamine |
| 1219. | N-[(1-butyl-2,4-diphenyl-1H-imidazol-5-yl)methyl]-N-[3- |
| | (trifluoromethoxy)benzyl]butan-1-amine |
| 1220. | N-benzyl-1-(1-butyl-2,4-diphenyl-1H-imidazol-5-yl)-N-[3- |
| | (trifluoromethoxy)benzyl]methanamine |
| 1221. | 1-(1-butyl-2,4-diphenyl-1H-imidazol-5-yl)-N-(4-methoxybenzyl)-N-[3- |
| | (trifluoromethoxy)benzyl]methanamine |
| 1222. | 1-(1-butyl-2,4-diphenyl-1H-imidazol-5-yl)-N-(cyclohexylmethyl)-N-[3- |
| | (trifluoromethoxy)benzyl]methanamine |
| 1223. | N-[(1-butyl-2,4-diphenyl-1H-imidazol-5-yl)methyl]-N-[4- |
| | (difluoromethoxy)benzyl]butan-1-amine |
| 1224. | N-benzyl-1-(1-butyl-2,4-diphenyl-1H-imidazol-5-yl)-N-[4- |
| | (difluoromethoxy)benzyl]methanamine |
| 1225. | 1-(1-butyl-2,4-diphenyl-1H-imidazol-5-yl)-N-[4-(difluoromethoxy)benzyl]-N-(4- |
| | methoxybenzyl)methanamine |
| 1226. | 1-(1-butyl-2,4-diphenyl-1H-imidazol-5-yl)-N-(cyclohexylmethyl)-N-[4- |
| | (difluoromethoxy)benzyl]methanamine |
| 1227. | N-[(1-butyl-2,4-diphenyl-1H-imidazol-5-yl)methyl]-N-[4- |
| | (trifluoromethoxy)benzyl]butan-1-amine |
| 1228. | N-benzyl-1-(1-butyl-2,4-diphenyl-1H-imidazol-5-yl)-N-[4- |
| | (trifluoromethoxy)benzyl]methanamine |
| 1229. | 1-(1-butyl-2,4-diphenyl-1H-imidazol-5-yl)-N-(4-methoxybenzyl)-N-[4- |
| | (trifluoromethoxy)benzyl]methanamine |
| 1230. | 1-(1-butyl-2,4-diphenyl-1H-imidazol-5-yl)-N-(cyclohexylmethyl)-N-[4- |
| | (trifluoromethoxy)benzyl]methanamine |
| 1231. | N-[(1-butyl-2,4-diphenyl-1H-imidazol-5-yl)methyl]-N-(3,5-difluorobenzyl)butan-1- |
| | amine |
| 1232. | N-benzyl-1-(1-butyl-2,4-diphenyl-1H-imidazol-5-yl)-N-(3,5- |
| | difluorobenzyl)methanamine |
| 1233. | 1-(1-butyl-2,4-diphenyl-1H-imidazol-5-yl)-N-(3,5-difluorobenzyl)-N-(4- |
| | methoxybenzyl)methanamine |
| 1234. | 1-(1-butyl-2,4-diphenyl-1H-imidazol-5-yl)-N-(cyclohexylmethyl)-N-(3,5- |
| | difluorobenzyl)methanamine |
| 1235. | N-[(1-butyl-2,4-diphenyl-1H-imidazol-5-yl)methyl]-N-(3-chloro-4-fluorobenzyl)butan- |
| | 1-amine |
| 1236. | N-benzyl-1-(1-butyl-2,4-diphenyl-1H-imidazol-5-yl)-N-(3-chloro-4- |
| | fluorobenzyl)methanamine |
| 1237. | 1-(1-butyl-2,4-diphenyl-1H-imidazol-5-yl)-N-(3-chloro-4-fluorobenzyl)-N-(4- |
| | methoxybenzyl)methanamine |
| 1238. | 1-(1-butyl-2,4-diphenyl-1H-imidazol-5-yl)-N-(3-chloro-4-fluorobenzyl)-N- |
| | (cyclohexylmethyl)methanamine |
| 1239. | N-[(1-butyl-2,4-diphenyl-1H-imidazol-5-yl)methyl]-N-[4-fluoro-3- |
| | (trifluoromethyl)benzyl]butan-1-amine |
| 1240. | N-benzyl-1-(1-butyl-2,4-diphenyl-1H-imidazol-5-yl)-N-[4-fluoro-3- |
| | (trifluoromethyl)benzyl]methanamine |
| 1241. | 1-(1-butyl-2,4-diphenyl-1H-imidazol-5-yl)-N-[4-fluoro-3-(trifluoromethyl)benzyl]-N- |
| | (4-methoxybenzyl)methanamine |
| 1242. | 1-(1-butyl-2,4-diphenyl-1H-imidazol-5-yl)-N-(cyclohexylmethyl)-N-[4-fluoro-3- |
| | (trifluoromethyl)benzyl]methanamine |
| 1243. | N-(3-bromo-4-fluorobenzyl)-N-[(1-butyl-2,4-diphenyl-1H-imidazol-5-yl)methyl]butan- |
| | 1-amine |
| 1244. | N-benzyl-1-(3-bromo-4-fluorophenyl)-N-[(1-butyl-2,4-diphenyl-1H-imidazol-5- |
| | yl)methyl]methanamine |
| 1245. | 1-(3-bromo-4-fluorophenyl)-N-[(1-butyl-2,4-diphenyl-1H-imidazol-5-yl)methyl]-N-(4- |
| | methoxybenzyl)methanamine |
| 1246. | 1-(3-bromo-4-fluorophenyl)-N-[(1-butyl-2,4-diphenyl-1H-imidazol-5-yl)methyl]-N- |
| | (cyclohexylmethyl)methanamine |
| 1247. | N-(3-bromo-4-methoxybenzyl)-N-[(1-butyl-2,4-diphenyl-1H-imidazol-5- |
| | yl)methyl]butan-1-amine |
| 1248. | N-benzyl-1-(3-bromo-4-methoxyphenyl)-N-[(1-butyl-2,4-diphenyl-1H-imidazol-5- |
| | yl)methyl]methanamine |
| 1249. | N-(3-bromo-4-methoxybenzyl)-N-[(1-butyl-2,4-diphenyl-1H-imidazol-5-yl)methyl]-N- |
| | (4-methoxybenzyl)amine |
| 1250. | N-(3-bromo-4-methoxybenzyl)-N-[(1-butyl-2,4-diphenyl-1H-imidazol-5-yl)methyl]-N- |
| | (cyclohexylmethyl)amine |
| 1251. | N-[(1-butyl-2,4-diphenyl-1H-imidazol-5-yl)methyl]-N-(4-propoxybenzyl)butan-1- |
| | amine |
| 1252. | N-benzyl-1-(1-butyl-2,4-diphenyl-1H-imidazol-5-yl)-N-(4- |
| | propoxybenzyl)methanamine |
| 1253. | 1-(1-butyl-2,4-diphenyl-1H-imidazol-5-yl)-N-(4-methoxybenzyl)-N-(4- |
| | propoxybenzyl)methanamine |
| 1254. | 1-(1-butyl-2,4-diphenyl-1H-imidazol-5-yl)-N-(cyclohexylmethyl)-N-(4- |
| | propoxybenzyl)methanamine |
| 1255. | N-(4-tert-butylbenzyl)-N-[(1-butyl-2,4-diphenyl-1H-imidazol-5-yl)methyl]butan-1- |
| | amine |
| 1256. | N-benzyl-N-(4-tert-butylbenzyl)-1-(1-butyl-2,4-diphenyl-1H-imidazol-5- |
| | yl)methanamine |
| 1257. | N-(4-tert-butylbenzyl)-1-(1-butyl-2,4-diphenyl-1H-imidazol-5-yl)-N-(4- |
| | methoxybenzyl)methanamine |
| 1258. | N-(4-tert-butylbenzyl)-1-(1-butyl-2,4-diphenyl-1H-imidazol-5-yl)-N- |
| | (cyclohexylmethyl)methanamine |
| 1259. | N-[(1-butyl-2,4-diphenyl-1H-imidazol-5-yl)methyl]-N-(3-fluoro-4- |
| | methoxybenzyl)butan-1-amine |
| 1260. | N-benzyl-1-(1-butyl-2,4-diphenyl-1H-imidazol-5-yl)-N-(3-fluoro-4- |
| | methoxybenzyl)methanamine |
| 1261. | 1-(1-butyl-2,4-diphenyl-1H-imidazol-5-yl)-N-(3-fluoro-4-methoxybenzyl)-N-(4- |
| | methoxybenzyl)methanamine |
| 1262. | 1-(1-butyl-2,4-diphenyl-1H-imidazol-5-yl)-N-(cyclohexylmethyl)-N-(3-fluoro-4- |
| | methoxybenzyl)methanamine |
| 1263. | N-benzyl-N-[(1-butyl-2,4-diphenyl-1H-imidazol-5-yl)methyl]ethanamine |
| 1264. | N-benzyl-N-[(1-butyl-2,4-diphenyl-1H-imidazol-5-yl)methyl]propan-1-amine |
| 1265. | N-(2-bromobenzyl)-N-[(1-butyl-2,4-diphenyl-1H-imidazol-5-yl)methyl]ethanamine |
| 1266. | N-(2-bromobenzyl)-N-[(1-butyl-2,4-diphenyl-1H-imidazol-5-yl)methyl]propan-1-amine |
| 1267. | N-[(1-butyl-2,4-diphenyl-1H-imidazol-5-yl)methyl]-N-(2-fluorobenzyl)ethanamine |
| 1268. | N-[(1-butyl-2,4-diphenyl-1H-imidazol-5-yl)methyl]-N-(2-fluorobenzyl)propan-1-amine |
| 1269. | N-[(1-butyl-2,4-diphenyl-1H-imidazol-5-yl)methyl]-N-(2-chlorobenzyl)ethanamine |
| 1270. | N-[(1-butyl-2,4-diphenyl-1H-imidazol-5-yl)methyl]-N-(2-chlorobenzyl)propan-1-amine |
| 1271. | N-[(1-butyl-2,4-diphenyl-1H-imidazol-5-yl)methyl]-N-(2-methylbenzyl)ethanamine |
| 1272. | N-[(1-butyl-2,4-diphenyl-1H-imidazol-5-yl)methyl]-N-(2-methylbenzyl)propan-1- |
| | amine |
| 1273. | N-[(1-butyl-2,4-diphenyl-1H-imidazol-5-yl)methyl]-N-(2-methoxybenzyl)propan-1- |
| | amine |
| 1274. | N-[(1-butyl-2,4-diphenyl-1H-imidazol-5-yl)methyl]-N-[2- |
| | (trifluoromethyl)benzyl]ethanamine |
| 1275. | N-[(1-butyl-2,4-diphenyl-1H-imidazol-5-yl)methyl]-N-[2- |
| | (trifluoromethyl)benzyl]propan-1-amine |
| 1276. | N-[(1-butyl-2,4-diphenyl-1H-imidazol-5-yl)methyl]-N-(2-ethoxybenzyl)ethanamine |
| 1277. | N-[(1-butyl-2,4-diphenyl-1H-imidazol-5-yl)methyl]-N-(2-ethoxybenzyl)propan-1- |
| | amine |
| 1278. | N-[(1-butyl-2,4-diphenyl-1H-imidazol-5-yl)methyl]-N-(2,4-dichlorobenzyl)propan-1- |
| | amine |
| 1279. | N-[(1-butyl-2,4-diphenyl-1H-imidazol-5-yl)methyl]-N-(2,4-dimethoxybenzyl)propan-1- |
| | amine |
| 1280. | N-[(1-butyl-2,4-diphenyl-1H-imidazol-5-yl)methyl]-N-(2,5- |
| | dimethoxybenzyl)ethanamine |
| 1281. | N-[(1-butyl-2,4-diphenyl-1H-imidazol-5-yl)methyl]-N-(2,5-dimethoxybenzyl)propan-1- |
| | amine |
| 1282. | N-(3-bromobenzyl)-N-[(1-butyl-2,4-diphenyl-1H-imidazol-5-yl)methyl]ethanamine |
| 1283. | N-(3-bromobenzyl)-N-[(1-butyl-2,4-diphenyl-1H-imidazol-5-yl)methyl]propan-1-amine |
| 1284. | N-(3-bromobenzyl)-N-[(1-butyl-2,4-diphenyl-1H-imidazol-5-yl)methyl]butan-1-amine |
| 1285. | N-[(1-butyl-2,4-diphenyl-1H-imidazol-5-yl)methyl]-N-(3-fluorobenzyl)ethanamine |
| 1286. | N-[(1-butyl-2,4-diphenyl-1H-imidazol-5-yl)methyl]-N-(3-fluorobenzyl)propan-1-amine |
| 1287. | N-[(1-butyl-2,4-diphenyl-1H-imidazol-5-yl)methyl]-N-(3-fluorobenzyl)butan-1-amine |
| 1288. | N-[(1-butyl-2,4-diphenyl-1H-imidazol-5-yl)methyl]-N-(3-methylbenzyl)ethanamine |
| 1289. | N-[(1-butyl-2,4-diphenyl-1H-imidazol-5-yl)methyl]-N-(3-methylbenzyl)propan-1- |
| | amine |
| 1290. | N-[(1-butyl-2,4-diphenyl-1H-imidazol-5-yl)methyl]-N-(3-methylbenzyl)butan-1-amine |
| 1291. | N-[(1-butyl-2,4-diphenyl-1H-imidazol-5-yl)methyl]-N-[3- |
| | (trifluoromethyl)benzyl]ethanamine |
| 1292. | N-[(1-butyl-2,4-diphenyl-1H-imidazol-5-yl)methyl]-N-[3- |
| | (trifluoromethyl)benzyl]propan-1-amine |
| 1293. | N-[(1-butyl-2,4-diphenyl-1H-imidazol-5-yl)methyl]-N-[3- |
| | (trifluoromethyl)benzyl]butan-1-amine |
| 1294. | N-[(1-butyl-2,4-diphenyl-1H-imidazol-5-yl)methyl]-N-(3-methoxybenzyl)ethanamine |
| 1295. | N-[(1-butyl-2,4-diphenyl-1H-imidazol-5-yl)methyl]-N-(3-methoxybenzyl)propan-1- |
| | amine |
| 1296. | N-[(1-butyl-2,4-diphenyl-1H-imidazol-5-yl)methyl]-N-(3-methoxybenzyl)butan-1- |
| | amine |
| 1297. | N-[(1-butyl-2,4-diphenyl-1H-imidazol-5-yl)methyl]-N-(3-chlorobenzyl)ethanamine |
| 1298. | N-[(1-butyl-2,4-diphenyl-1H-imidazol-5-yl)methyl]-N-(3-chlorobenzyl)propan-1-amine |
| 1299. | N-[(1-butyl-2,4-diphenyl-1H-imidazol-5-yl)methyl]-N-(3-chlorobenzyl)butan-1-amine |
| 1300. | N-[(1-butyl-2,4-diphenyl-1H-imidazol-5-yl)methyl]-N-(3-phenoxybenzyl)ethanamine |
| 1301. | N-[(1-butyl-2,4-diphenyl-1H-imidazol-5-yl)methyl]-N-(3-phenoxybenzyl)propan-1- |
| | amine |
| 1302. | N-[(1-butyl-2,4-diphenyl-1H-imidazol-5-yl)methyl]-N-(3-phenoxybenzyl)butan-1- |
| | amine |
| 1303. | N-[(1-butyl-2,4-diphenyl-1H-imidazol-5-yl)methyl]-N-[(2,2-difluoro-1,3-benzodioxol- |
| | 5-yl)methyl]ethanamine |
| 1304. | N-[(1-butyl-2,4-diphenyl-1H-imidazol-5-yl)methyl]-N-[(2,2-difluoro-1,3-benzodioxol- |
| | 5-yl)methyl]propan-1-amine |
| 1305. | N-[(1-butyl-2,4-diphenyl-1H-imidazol-5-yl)methyl]-N-[(2,2-difluoro-1,3-benzodioxol- |
| | 5-yl)methyl]butan-1-amine |
| 1306. | 1-(4-bromophenyl)-N-[(1-butyl-2,4-diphenyl-1H-imidazol-5-yl)methyl]-N- |
| | methylmethanamine |
| 1307. | N-(4-bromobenzyl)-N-[(1-butyl-2,4-diphenyl-1H-imidazol-5-yl)methyl]ethanamine |
| 1308. | N-(4-bromobenzyl)-N-[(1-butyl-2,4-diphenyl-1H-imidazol-5-yl)methyl]propan-1-amine |
| 1309. | N-[(1-butyl-2,4-diphenyl-1H-imidazol-5-yl)methyl]-N-(4-fluorobenzyl)ethanamine |
| 1310. | N-[(1-butyl-2,4-diphenyl-1H-imidazol-5-yl)methyl]-N-(4-fluorobenzyl)propan-1-amine |
| 1311. | N-[(1-butyl-2,4-diphenyl-1H-imidazol-5-yl)methyl]-N-(4-chlorobenzyl)ethanamine |
| 1312. | N-[(1-butyl-2,4-diphenyl-1H-imidazol-5-yl)methyl]-N-(4-chlorobenzyl)propan-1-amine |
| 1313. | N-[(1-butyl-2,4-diphenyl-1H-imidazol-5-yl)methyl]-N-(4-methylbenzyl)ethanamine |
| 1314. | N-[(1-butyl-2,4-diphenyl-1H-imidazol-5-yl)methyl]-N-(4-methylbenzyl)propan-1- |
| | amine |
| 1315. | 1-(1-butyl-2,4-diphenyl-1H-imidazol-5-yl)-N-(4-ethylbenzyl)-N-methylmethanamine |
| 1316. | N-[(1-butyl-2,4-diphenyl-1H-imidazol-5-yl)methyl]-N-(4-ethylbenzyl)ethanamine |
| 1317. | N-[(1-butyl-2,4-diphenyl-1H-imidazol-5-yl)methyl]-N-(4-ethylbenzyl)propan-1-amine |
| 1318. | N-[(1-butyl-2,4-diphenyl-1H-imidazol-5-yl)methyl]-N-(4-methoxybenzyl)ethanamine |
| 1319. | N-[(1-butyl-2,4-diphenyl-1H-imidazol-5-yl)methyl]-N-(4-methoxybenzyl)propan-1- |
| | amine |
| 1320. | N-[(1-butyl-2,4-diphenyl-1H-imidazol-5-yl)methyl]-N-(4-ethoxybenzyl)ethanamine |
| 1321. | N-[(1-butyl-2,4-diphenyl-1H-imidazol-5-yl)methyl]-N-(4-ethoxybenzyl)propan-1- |
| | amine |
| 1322. | 1-(4-butoxyphenyl)-N-[(1-butyl-2,4-diphenyl-1H-imidazol-5-yl)methyl]-N- |
| | methylmethanamine |
| 1323. | N-(4-butoxybenzyl)-N-[(1-butyl-2,4-diphenyl-1H-imidazol-5-yl)methyl]ethanamine |
| 1324. | N-(4-butoxybenzyl)-N-[(1-butyl-2,4-diphenyl-1H-imidazol-5-yl)methyl]propan-1- |
| | amine |
| 1325. | N-[(1-butyl-2,4-diphenyl-1H-imidazol-5-yl)methyl]-N-[4- |
| | (trifluoromethyl)benzyl]propan-1-amine |
| 1326. | 1-(1-butyl-2,4-diphenyl-1H-imidazol-5-yl)-N-(4-isopropylbenzyl)-N- |
| | methylmethanamine |
| 1327. | N-[(1-butyl-2,4-diphenyl-1H-imidazol-5-yl)methyl]-N-(4-isopropylbenzyl)ethanamine |
| 1328. | N-[(1-butyl-2,4-diphenyl-1H-imidazol-5-yl)methyl]-N-(4-isopropylbenzyl)propan-1- |
| | amine |
| 1329. | N-(1,1′-biphenyl-4-ylmethyl)-N-[(1-butyl-2,4-diphenyl-1H-imidazol-5- |
| | yl)methyl]propan-1-amine |
| 1330. | 1-(1,3-benzodioxol-5-yl)-N-[(1-butyl-2,4-diphenyl-1H-imidazol-5-yl)methyl]-N- |
| | methylmethanamine |
| 1331. | N-(1,3-benzodioxol-5-ylmethyl)-N-[(1-butyl-2,4-diphenyl-1H-imidazol-5- |
| | yl)methyl]ethanamine |
| 1332. | N-(1,3-benzodioxol-5-ylmethyl)-N-[(1-butyl-2,4-diphenyl-1H-imidazol-5- |
| | yl)methyl]propan-1-amine |
| 1333. | N-[(1-butyl-2,4-diphenyl-1H-imidazol-5-yl)methyl]-N-(2,3-dihydro-1,4-benzodioxin-6- |
| | ylmethyl)ethanamine |
| 1334. | N-[(1-butyl-2,4-diphenyl-1H-imidazol-5-yl)methyl]-N-(2,3-dihydro-1,4-benzodioxin-6- |
| | ylmethyl)propan-1-amine |
| 1335. | N-[(1-butyl-2,4-diphenyl-1H-imidazol-5-yl)methyl]-N-(4-phenoxybenzyl)ethanamine |
| 1336. | N-[(1-butyl-2,4-diphenyl-1H-imidazol-5-yl)methyl]-N-(4-phenoxybenzyl)propan-1- |
| | amine |
| 1337. | N-[4-(benzyloxy)benzyl]-N-[(1-butyl-2,4-diphenyl-1H-imidazol-5- |
| | yl)methyl]ethanamine |
| 1338. | N-[4-(benzyloxy)benzyl]-N-[(1-butyl-2,4-diphenyl-1H-imidazol-5-yl)methyl]propan-1- |
| | amine |
| 1339. | N-[3-(benzyloxy)-4-methoxybenzyl]-N-[(1-butyl-2,4-diphenyl-1H-imidazol-5- |
| | yl)methyl]propan-1-amine |
| 1340. | N-[4-(benzyloxy)-3-methoxybenzyl]-N-[(1-butyl-2,4-diphenyl-1H-imidazol-5- |
| | yl)methyl]butan-1-amine |
| 1341. | N-[(1-butyl-2,4-diphenyl-1H-imidazol-5-yl)methyl]-N-(3,4-diethoxybenzyl)propan-1- |
| | amine |
| 1342. | N-[(1-butyl-2,4-diphenyl-1H-imidazol-5-yl)methyl]-N-(2,3-dimethoxybenzyl)propan-1- |
| | amine |
| 1343. | N-benzyl-N-[(2,4-diphenyl-1-propyl-1H-imidazol-5-yl)methyl]butan-1-amine |
| 1344. | N-benzyl-N-[(1-pentyl-2,4-diphenyl-1H-imidazol-5-yl)methyl]butan-1-amine |
| 1345. | N-(2-bromobenzyl)-N-[(2,4-diphenyl-1-propyl-1H-imidazol-5-yl)methyl]butan-1-amine |
| 1346. | N-(2-bromobenzyl)-N-[(1-pentyl-2,4-diphenyl-1H-imidazol-5-yl)methyl]butan-1-amine |
| 1347. | N-[(2,4-diphenyl-1-propyl-1H-imidazol-5-yl)methyl]-N-(2-fluorobenzyl)butan-1-amine |
| 1348. | N-(2-fluorobenzyl)-N-[(1-pentyl-2,4-diphenyl-1H-imidazol-5-yl)methyl]butan-1-amine |
| 1349. | N-(2-chlorobenzyl)-N-[(1-ethyl-2,4-diphenyl-1H-imidazol-5-yl)methyl]butan-1-amine |
| 1350. | N-(2-chlorobenzyl)-N-[(2,4-diphenyl-1-propyl-1H-imidazol-5-yl)methyl]butan-1-amine |
| 1351. | N-(2-chlorobenzyl)-N-[(1-pentyl-2,4-diphenyl-1H-imidazol-5-yl)methyl]butan-1-amine |
| 1352. | N-(2-methylbenzyl)-N-[(1-pentyl-2,4-diphenyl-1H-imidazol-5-yl)methyl]butan-1- |
| | amine |
| 1353. | N-[(2,4-diphenyl-1-propyl-1H-imidazol-5-yl)methyl]-N-(2-methoxybenzyl)butan-1- |
| | amine |
| 1354. | N-(2-methoxybenzyl)-N-[(1-pentyl-2,4-diphenyl-1H-imidazol-5-yl)methyl]butan-1- |
| | amine |
| 1355. | N-[(2,4-diphenyl-1-propyl-1H-imidazol-5-yl)methyl]-N-[2- |
| | (trifluoromethyl)benzyl]butan-1-amine |
| 1356. | N-[(2,4-diphenyl-1-propyl-1H-imidazol-5-yl)methyl]-N-(2-ethoxybenzyl)butan-1- |
| | amine |
| 1357. | N-(2-ethoxybenzyl)-N-[(1-pentyl-2,4-diphenyl-1H-imidazol-5-yl)methyl]butan-1-amine |
| 1358. | N-(2,4-dimethoxybenzyl)-N-[(2,4-diphenyl-1-propyl-1H-imidazol-5-yl)methyl]butan-1- |
| | amine |
| 1359. | N-(2,4-dimethoxybenzyl)-N-[(1-pentyl-2,4-diphenyl-1H-imidazol-5-yl)methyl]butan-1- |
| | amine |
| 1360. | N-(2,5-dimethoxybenzyl)-N-[(2,4-diphenyl-1-propyl-1H-imidazol-5-yl)methyl]butan-1- |
| | amine |
| 1361. | N-(2,5-dimethoxybenzyl)-N-[(1-pentyl-2,4-diphenyl-1H-imidazol-5-yl)methyl]butan-1- |
| | amine |
| 1362. | N-[(2,4-diphenyl-1-propyl-1H-imidazol-5-yl)methyl]-N-(3-fluorobenzyl)butan-1-amine |
| 1363. | N-[(2,4-diphenyl-1-propyl-1H-imidazol-5-yl)methyl]-N-(3-methylbenzyl)butan-1- |
| | amine |
| 1364. | N-(3-methylbenzyl)-N-[(1-pentyl-2,4-diphenyl-1H-imidazol-5-yl)methyl]butan-1- |
| | amine |
| 1365. | N-[(2,4-diphenyl-1-propyl-1H-imidazol-5-yl)methyl]-N-(3-methoxybenzyl)butan-1- |
| | amine |
| 1366. | N-(3-methoxybenzyl)-N-[(1-pentyl-2,4-diphenyl-1H-imidazol-5-yl)methyl]butan-1- |
| | amine |
| 1367. | N-(3-chlorobenzyl)-N-[(2,4-diphenyl-1-propyl-1H-imidazol-5-yl)methyl]butan-1-amine |
| 1368. | N-(4-bromobenzyl)-N-[(2,4-diphenyl-1-propyl-1H-imidazol-5-yl)methyl]butan-1-amine |
| 1369. | N-(4-bromobenzyl)-N-[(1-pentyl-2,4-diphenyl-1H-imidazol-5-yl)methyl]butan-1-amine |
| 1370. | N-[(2,4-diphenyl-1-propyl-1H-imidazol-5-yl)methyl]-N-(4-fluorobenzyl)butan-1-amine |
| 1371. | N-(4-fluorobenzyl)-N-[(1-pentyl-2,4-diphenyl-1H-imidazol-5-yl)methyl]butan-1-amine |
| 1372. | N-(4-chlorobenzyl)-N-[(2,4-diphenyl-1-propyl-1H-imidazol-5-yl)methyl]butan-1-amine |
| 1373. | N-(4-chlorobenzyl)-N-[(1-pentyl-2,4-diphenyl-1H-imidazol-5-yl)methyl]butan-1-amine |
| 1374. | N-[(1-ethyl-2,4-diphenyl-1H-imidazol-5-yl)methyl]-N-(4-methylbenzyl)butan-1-amine |
| 1375. | N-[(2,4-diphenyl-1-propyl-1H-imidazol-5-yl)methyl]-N-(4-methylbenzyl)butan-1- |
| | amine |
| 1376. | N-(4-methylbenzyl)-N-[(1-pentyl-2,4-diphenyl-1H-imidazol-5-yl)methyl]butan-1- |
| | amine |
| 1377. | N-(4-ethylbenzyl)-N-[(1-methyl-2,4-diphenyl-1H-imidazol-5-yl)methyl]butan-1-amine |
| 1378. | N-(4-ethylbenzyl)-N-[(1-ethyl-2,4-diphenyl-1H-imidazol-5-yl)methyl]butan-1-amine |
| 1379. | N-[(2,4-diphenyl-1-propyl-1H-imidazol-5-yl)methyl]-N-(4-ethylbenzyl)butan-1-amine |
| 1380. | N-(4-ethylbenzyl)-N-[(1-pentyl-2,4-diphenyl-1H-imidazol-5-yl)methyl]butan-1-amine |
| 1381. | N-[(2,4-diphenyl-1-propyl-1H-imidazol-5-yl)methyl]-N-(4-methoxybenzyl)butan-1- |
| | amine |
| 1382. | N-(4-methoxybenzyl)-N-[(1-pentyl-2,4-diphenyl-1H-imidazol-5-yl)methyl]butan-1- |
| | amine |
| 1383. | N-(4-ethoxybenzyl)-N-[(1-ethyl-2,4-diphenyl-1H-imidazol-5-yl)methyl]butan-1-amine |
| 1384. | N-[(2,4-diphenyl-1-propyl-1H-imidazol-5-yl)methyl]-N-(4-ethoxybenzyl)butan-1- |
| | amine |
| 1385. | N-(4-ethoxybenzyl)-N-[(1-pentyl-2,4-diphenyl-1H-imidazol-5-yl)methyl]butan-1-amine |
| 1386. | N-(4-butoxybenzyl)-N-[(2,4-diphenyl-1-propyl-1H-imidazol-5-yl)methyl]butan-1- |
| | amine |
| 1387. | N-(4-butoxybenzyl)-N-[(1-pentyl-2,4-diphenyl-1H-imidazol-5-yl)methyl]butan-1- |
| | amine |
| 1388. | N-[(1-pentyl-2,4-diphenyl-1H-imidazol-5-yl)methyl]-N-[4- |
| | (trifluoromethyl)benzyl]butan-1-amine |
| 1389. | N-[(1-ethyl-2,4-diphenyl-1H-imidazol-5-yl)methyl]-N-(4-isopropylbenzyl)butan-1- |
| | amine |
| 1390. | N-[(2,4-diphenyl-1-propyl-1H-imidazol-5-yl)methyl]-N-(4-isopropylbenzyl)butan-1- |
| | amine |
| 1391. | N-(4-isopropylbenzyl)-N-[(1-pentyl-2,4-diphenyl-1H-imidazol-5-yl)methyl]butan-1- |
| | amine |
| 1392. | N-(1,3-benzodioxol-5-ylmethyl)-N-[(1-ethyl-2,4-diphenyl-1H-imidazol-5- |
| | yl)methyl]butan-1-amine |
| 1393. | N-(1,3-benzodioxol-5-ylmethyl)-N-[(2,4-diphenyl-1-propyl-1H-imidazol-5- |
| | yl)methyl]butan-1-amine |
| 1394. | N-(1,3-benzodioxol-5-ylmethyl)-N-[(1-pentyl-2,4-diphenyl-1H-imidazol-5- |
| | yl)methyl]butan-1-amine |
| 1395. | N-(2,3-dihydro-1,4-benzodioxin-6-ylmethyl)-N-[(1-ethyl-2,4-diphenyl-1H-imidazol-5- |
| | yl)methyl]butan-1-amine |
| 1396. | N-(2,3-dihydro-1,4-benzodioxin-6-ylmethyl)-N-[(2,4-diphenyl-1-propyl-1H-imidazol- |
| | 5-yl)methyl]butan-1-amine |
| 1397. | N-(2,3-dihydro-1,4-benzodioxin-6-ylmethyl)-N-[(1-pentyl-2,4-diphenyl-1H-imidazol-5- |
| | yl)methyl]butan-1-amine |
| 1398. | N-[(1-pentyl-2,4-diphenyl-1H-imidazol-5-yl)methyl]-N-(4-phenoxybenzyl)butan-1- |
| | amine |
| 1399. | N-[4-(benzyloxy)benzyl]-N-[(1-pentyl-2,4-diphenyl-1H-imidazol-5-yl)methyl]butan-1- |
| | amine |
| 1400. | N-[3-(benzyloxy)-4-methoxybenzyl]-N-[(1-pentyl-2,4-diphenyl-1H-imidazol-5- |
| | yl)methyl]butan-1-amine |
| 1401. | N-(3,4-dimethoxybenzyl)-N-[(1-pentyl-2,4-diphenyl-1H-imidazol-5-yl)methyl]butan-1- |
| | amine |
| 1402. | N-[(2,4-diphenyl-1-propyl-1H-imidazol-5-yl)methyl]-N-[(6-nitro-1,3-benzodioxol-5- |
| | yl)methyl]butan-1-amine |
| 1403. | N-[(6-nitro-1,3-benzodioxol-5-yl)methyl]-N-[(1-pentyl-2,4-diphenyl-1H-imidazol-5- |
| | yl)methyl]butan-1-amine |
| 1404. | N-(2,4-dimethylbenzyl)-N-[(1-ethyl-2,4-diphenyl-1H-imidazol-5-yl)methyl]butan-1- |
| | amine |
| 1405. | N-(2,4-dimethylbenzyl)-N-[(2,4 diphenyl-1-propyl-1H-imidazol-5-yl)methyl]butan-1- |
| | amine |
| 1406. | N-(2,4-dimethylbenzyl)-N-[(1-pentyl-2,4-diphenyl-1H-imidazol-5-yl)methyl]butan-1- |
| | amine |
| 1407. | N-(2,5-difluorobenzyl)-N-[(2,4-diphenyl-1-propyl-1H-imidazol-5-yl)methyl]butan-1- |
| | amine |
| 1408. | N-(2,5-difluorobenzyl)-N-[(1-pentyl-2,4-diphenyl-1H-imidazol-5-yl)methyl]butan-1- |
| | amine |
| 1409. | N-(2,6-dichlorobenzyl)-N-[(1-ethyl-2,4-diphenyl-1H-imidazol-5-yl)methyl]butan-1- |
| | amine |
| 1410. | N-(2,6-dichlorobenzyl)-N-[(2,4-diphenyl-1-propyl-1H-imidazol-5-yl)methyl]butan-1- |
| | amine |
| 1411. | N-(2,6-dichlorobenzyl)-N-[(1-pentyl-2,4-diphenyl-1H-imidazol-5-yl)methyl]butan-1- |
| | amine |
| 1412. | N-(2-chloro-6-fluorobenzyl)-N-[(1-ethyl-2,4-diphenyl-1H-imidazol-5-yl)methyl]butan- |
| | 1-amine |
| 1413. | N-(2-chloro-6-fluorobenzyl)-N-[(2,4-diphenyl-1-propyl-1H-imidazol-5- |
| | yl)methyl]butan-1-amine |
| 1414. | N-(2-chloro-6-fluorobenzyl)-N-[(1-pentyl-2,4-diphenyl-1H-imidazol-5- |
| | yl)methyl]butan-1-amine |
| 1415. | N-(2,3-difluorobenzyl)-N-[(2,4-diphenyl-1-propyl-1H-imidazol-5-yl)methyl]butan-1- |
| | amine |
| 1416. | N-(2,3-difluorobenzyl)-N-[(1-pentyl-2,4-diphenyl-1H-imidazol-5-yl)methyl]butan-1- |
| | amine |
| 1417. | N-(2,6-difluorobenzyl)-N-[(1-methyl-2,4-diphenyl-1H-imidazol-5-yl)methyl]butan-1- |
| | amine |
| 1418. | N-(2,6-difluorobenzyl)-N-[(1-ethyl-2,4-diphenyl-1H-imidazol-5-yl)methyl]butan-1- |
| | amine |
| 1419. | N-(2,6-difluorobenzyl)-N-[(2,4-diphenyl-1-propyl-1H-imidazol-5-yl)methyl]butan-1- |
| | amine |
| 1420. | N-(2,6-difluorobenzyl)-N-[(1-pentyl-2,4-diphenyl-1H-imidazol-5-yl)methyl]butan-1- |
| | amine |
| 1421. | N-(2,4-difluorobenzyl)-N-[(1-methyl-2,4-diphenyl-1H-imidazol-5-yl)methyl]butan-1- |
| | amine |
| 1422. | N-(2,4-difluorobenzyl)-N-[(2,4-diphenyl-1-propyl-1H-imidazol-5-yl)methyl]butan-1- |
| | amine |
| 1423. | N-(2,4-difluorobenzyl)-N-[(1-pentyl-2,4-diphenyl-1H-imidazol-5-yl)methyl]butan-1- |
| | amine |
| 1424. | N-[(2,4-diphenyl-1-propyl-1H-imidazol-5-yl)methyl]-N-(2-fluoro-5- |
| | methoxybenzyl)butan-1-amine |
| 1425. | N-(2-fluoro-5-methoxybenzyl)-N-[(1-pentyl-2,4-diphenyl-1H-imidazol-5- |
| | yl)methyl]butan-1-amine |
| 1426. | N-[(2,4-diphenyl-1-propyl-1H-imidazol-5-yl)methyl]-N-(2,3,4-trimethoxybenzyl)butan- |
| | 1-amine |
| 1427. | N-[(1-pentyl-2,4-diphenyl-1H-imidazol-5-yl)methyl]-N-(2,3,4-trimethoxybenzyl)butan- |
| | 1-amine |
| 1428. | N-(1,3-benzodioxol-4-ylmethyl)-N-[(1-pentyl-2,4-diphenyl-1H-imidazol-5- |
| | yl)methyl]butan-1-amine |
| 1429. | N-[(1-ethyl-2,4-diphenyl-1H-imidazol-5-yl)methyl]-N-(4-methoxy-3- |
| | methylbenzyl)butan-1-amine |
| 1430. | N-[(2,4-diphenyl-1-propyl-1H-imidazol-5-yl)methyl]-N-(4-methoxy-3- |
| | methylbenzyl)butan-1-amine |
| 1431. | N-(4-methoxy-3-methylbenzyl)-N-[(1-pentyl-2,4-diphenyl-1H-imidazol-5- |
| | yl)methyl]butan-1-amine |
| 1432. | N-(2,4-dimethoxy-3-methylbenzyl)-N-[(2,4-diphenyl-1-propyl-1H-imidazol-5- |
| | yl)methyl]butan-1-amine |
| 1433. | N-(2,4-dimethoxy-3-methylbenzyl)-N-[(1-pentyl-2,4-diphenyl-1H-imidazol-5- |
| | yl)methyl]butan-1-amine |
| 1434. | N-[(2,4-diphenyl-1-propyl-1H-imidazol-5-yl)methyl]-N-(2,3,6-trifluorobenzyl)butan-1- |
| | amine |
| 1435. | N-[(2,4-diphenyl-1-propyl-1H-imidazol-5-yl)methyl]-N-[2-fluoro-6- |
| | (trifluoromethyl)benzyl]butan-1-amine |
| 1436. | N-[(1-ethyl-2,4-diphenyl-1H-imidazol-5-yl)methyl]-N-(4-methoxy-2,5- |
| | dimethylbenzyl)butan-1-amine |
| 1437. | N-[(2,4-diphenyl-1-propyl-1H-imidazol-5-yl)methyl]-N-(4-methoxy-2,5- |
| | dimethylbenzyl)butan-1-amine |
| 1438. | N-(4-methoxy-2,5-dimethylbenzyl)-N-[(1-pentyl-2,4-diphenyl-1H-imidazol-5- |
| | yl)methyl]butan-1-amine |
| 1439. | N-(2-chloro-3,4-dimethoxybenzyl)-N-[(2,4-diphenyl-1-propyl-1H-imidazol-5- |
| | yl)methyl]butan-1-amine |
| 1440. | N-(2-chloro-3,4-dimethoxybenzyl)-N-[(1-pentyl-2,4-diphenyl-1H-imidazol-5- |
| | yl)methyl]butan-1-amine |
| 1441. | N-(2-chloro-4-fluorobenzyl)-N-[(2,4-diphenyl-1-propyl-1H-imidazol-5- |
| | yl)methyl]butan-1-amine |
| 1442. | N-(2-chloro-4-fluorobenzyl)-N-[(1-pentyl-2,4-diphenyl-1H-imidazol-5- |
| | yl)methyl]butan-1-amine |
| 1443. | N-(2,6-dimethoxybenzyl)-N-[(1-pentyl-2,4-diphenyl-1H-imidazol-5-yl)methyl]butan-1- |
| | amine |
| 1444. | 2-({butyl[(1-pentyl-2,4-diphenyl-1H-imidazol-5-yl)methyl]amino}methyl)-6- |
| | methoxyphenol |
| 1445. | 2-({butyl[(2,4-diphenyl-1-propyl-1H-imidazol-5-yl)methyl]amino}methyl)-6- |
| | ethoxyphenol |
| 1446. | 2-({butyl[(1-pentyl-2,4-diphenyl-1H-imidazol-5-yl)methyl]amino}methyl)-6- |
| | ethoxyphenol |
| 1447. | 2-({butyl[(1-pentyl-2,4-diphenyl-1H-imidazol-5-yl)methyl]amino}methyl)-5- |
| | methoxyphenol |
| 1448. | 4-bromo-2-({butyl[(2,4-diphenyl-1-propyl-1H-imidazol-5-yl)methyl]amino}methyl)-6- |
| | methoxyphenol |
| 1449. | 4-bromo-2-({butyl[(1-pentyl-2,4-diphenyl-1H-imidazol-5-yl)methyl]amino}methyl)-6- |
| | methoxyphenol |
| 1450. | 2-({butyl[(2,4-diphenyl-1-propyl-1H-imidazol-5-yl)methyl]amino}methyl)-6- |
| | methylphenol |
| 1451. | 2-({butyl[(1-pentyl-2,4-diphenyl-1H-imidazol-5-yl)methyl]amino}methyl)-6- |
| | methylphenol |
| 1452. | 2-({butyl[(1-ethyl-2,4-diphenyl-1H-imidazol-5-yl)methyl]amino}methyl)-4- |
| | methylphenol |
| 1453. | 2-({butyl[(2,4-diphenyl-1-propyl-1H-imidazol-5-yl)methyl]amino}methyl)-4- |
| | methylphenol |
| 1454. | 4-({butyl[(2,4-diphenyl-1-propyl-1H-imidazol-5-yl)methyl]amino}methyl)-2- |
| | ethoxyphenol |
| 1455. | 4-({butyl[(1-pentyl-2,4-diphenyl-1H-imidazol-5-yl)methyl]amino}methyl)-2- |
| | ethoxyphenol |
| 1456. | 4-({butyl[(1-methyl-2,4-diphenyl-1H-imidazol-5-yl)methyl]amino}methyl)-2,6- |
| | dimethylphenol |
| 1457. | 4-({butyl[(1-ethyl-2,4-diphenyl-1H-imidazol-5-yl)methyl]amino}methyl)-2,6- |
| | dimethylphenol |
| 1458. | 4-({butyl[(2,4-diphenyl-1-propyl-1H-imidazol-5-yl)methyl]amino}methyl)-2,6- |
| | dimethylphenol |
| 1459. | 4-({butyl[(1-pentyl-2,4-diphenyl-1H-imidazol-5-yl)methyl]amino}methyl)-2,6- |
| | dimethylphenol |
| 1460. | 4-({butyl[(1-methyl-2,4-diphenyl-1H-imidazol-5-yl)methyl]amino}methyl)-2- |
| | methylphenol |
| 1461. | 4-({butyl[(1-ethyl-2,4-diphenyl-1H-imidazol-5-yl)methyl]amino}methyl)-2- |
| | methylphenol |
| 1462. | 4-({butyl[(2,4-diphenyl-1-propyl-1H-imidazol-5-yl)methyl]amino}methyl)-2- |
| | methylphenol |
| 1463. | 4-({butyl[(1-pentyl-2,4-diphenyl-1H-imidazol-5-yl)methyl]amino}methyl)-2- |
| | methylphenol |
| 1464. | N-(2-bromobenzyl)-N-{[1-butyl-4-(4-chlorophenyl)-2-phenyl-1H-imidazol-5- |
| | yl]methyl}butan-1-amine |
| 1465. | N-{[1-butyl-4-(4-chlorophenyl)-2-phenyl-1H-imidazol-5-yl]methyl}-N-(2- |
| | fluorobenzyl)butan-1-amine |
| 1466. | N-{[1-butyl-4-(4-methoxyphenyl)-2-phenyl-1H-imidazol-5-yl]methyl}-N-(2- |
| | fluorobenzyl)butan-1-amine |
| 1467. | N-{[1-butyl-4-(4-chlorophenyl)-2-phenyl-1H-imidazol-5-yl]methyl}-N-(2- |
| | chlorobenzyl)butan-1-amine |
| 1468. | N-{[1-butyl-4-(4-chlorophenyl)-2-phenyl-1H-imidazol-5-yl]methyl}-N-(2,4- |
| | dichlorobenzyl)butan-1-amine |
| 1469. | N-{[1-butyl-4-(4-chlorophenyl)-2-phenyl-1H-imidazol-5-yl]methyl}-N-(3- |
| | methylbenzyl)butan-1-amine |
| 1470. | N-{[1-butyl-4-(4-methoxyphenyl)-2-phenyl-1H-imidazol-5-yl]methyl}-N-(3- |
| | methylbenzyl)butan-1-amine |
| 1471. | N-{[1-butyl-4-(4-chlorophenyl)-2-phenyl-1H-imidazol-5-yl]methyl}-N-[3- |
| | (trifluoromethyl)benzyl]butan-1-amine |
| 1472. | N-{[1-butyl-4-(4-fluorophenyl)-2-phenyl-1H-imidazol-5-yl]methyl}-N-(3- |
| | methoxybenzyl)butan-1-amine |
| 1473. | N-{[1-butyl-4-(4-chlorophenyl)-2-phenyl-1H-imidazol-5-yl]methyl}-N-(3- |
| | methoxybenzyl)butan-1-amine |
| 1474. | N-{[1-butyl-4-(4-methoxyphenyl)-2-phenyl-1H-imidazol-5-yl]methyl}-N-(3- |
| | methoxybenzyl)butan-1-amine |
| 1475. | N-{[1-butyl-4-(4-chlorophenyl)-2-phenyl-1H-imidazol-5-yl]methyl}-N-(3- |
| | chlorobenzyl)butan-1-amine |
| 1476. | N-{[1-butyl-4-(4-chlorophenyl)-2-phenyl-1H-imidazol-5-yl]methyl}-N-(3- |
| | phenoxybenzyl)butan-1-amine |
| 1477. | N-{[1-butyl-4-(4-chlorophenyl)-2-phenyl-1H-imidazol-5-yl]methyl}-N-(4- |
| | chlorobenzyl)butan-1-amine |
| 1478. | N-{[1-butyl-4-(4-chlorophenyl)-2-phenyl-1H-imidazol-5-yl]methyl}-N-(4- |
| | methylbenzyl)butan-1-amine |
| 1479. | N-{[1-butyl-4-(4-methoxyphenyl)-2-phenyl-1H-imidazol-5-yl]methyl}-N-(4- |
| | methylbenzyl)butan-1-amine |
| 1480. | N-{[1-butyl-4-(4-chlorophenyl)-2-phenyl-1H-imidazol-5-yl]methyl}-N-(4- |
| | ethylbenzyl)butan-1-amine |
| 1481. | N-{[1-butyl-4-(4-methoxyphenyl)-2-phenyl-1H-imidazol-5-yl]methyl}-N-(4- |
| | ethylbenzyl)butan-1-amine |
| 1482. | N-{[1-butyl-4-(4-chlorophenyl)-2-phenyl-1H-imidazol-5-yl]methyl}-N-(4- |
| | methoxybenzyl)butan-1-amine |
| 1483. | N-{[1-butyl-4-(4-methoxyphenyl)-2-phenyl-1H-imidazol-5-yl]methyl}-N-(4- |
| | methoxybenzyl)butan-1-amine |
| 1484. | N-{[1-butyl-4-(4-chlorophenyl)-2-phenyl-1H-imidazol-5-yl]methyl}-N-(4- |
| | ethoxybenzyl)butan-1-amine |
| 1485. | N-{[1-butyl-4-(4-methoxyphenyl)-2-phenyl-1H-imidazol-5-yl]methyl}-N-(4- |
| | ethoxybenzyl)butan-1-amine |
| 1486. | N-(4-butoxybenzyl)-N-{[1-butyl-4-(4-chlorophenyl)-2-phenyl-1H-imidazol-5- |
| | yl]methyl}butan-1-amine |
| 1487. | N-(4-butoxybenzyl)-N-{[1-butyl-4-(4-methoxyphenyl)-2-phenyl-1H-imidazol-5- |
| | yl]methyl}butan-1-amine |
| 1488. | N-{[1-butyl-4-(4-chlorophenyl)-2-phenyl-1H-imidazol-5-yl]methyl}-N-[4- |
| | (trifluoromethyl)benzyl]butan-1-amine |
| 1489. | N-{[1-butyl-4-(4-chlorophenyl)-2-phenyl-1H-imidazol-5-yl]methyl}-N-(4- |
| | isopropylbenzyl)butan-1-amine |
| 1490. | N-{[1-butyl-4-(4-methoxyphenyl)-2-phenyl-1H-imidazol-5-yl]methyl}-N-(4- |
| | isopropylbenzyl)butan-1-amine |
| 1491. | N-(1,1′-biphenyl-4-ylmethyl)-N-{[1-butyl-4-(4-chlorophenyl)-2-phenyl-1H-imidazol-5- |
| | yl]methyl}butan-1-amine |
| 1492. | N-(1,3-benzodioxol-5-ylmethyl)-N-{[1-butyl-4-(4-fluorophenyl)-2-phenyl-1H- |
| | imidazol-5-yl]methyl}butan-1-amine |
| 1493. | N-(1,3-benzodioxol-5-ylmethyl)-N-{[1-butyl-4-(4-chlorophenyl)-2-phenyl-1H- |
| | imidazol-5-yl]methyl}butan-1-amine |
| 1494. | N-(1,3-benzodioxol-5-ylmethyl)-N-{[1-butyl-4-(4-methoxyphenyl)-2-phenyl-1H- |
| | imidazol-5-yl]methyl}butan-1-amine |
| 1495. | N-(1,3-benzodioxol-5-ylmethyl)-N-{[1-butyl-4-(2-methoxyphenyl)-2-phenyl-1H- |
| | imidazol-5-yl]methyl}butan-1-amine |
| 1496. | N-{[1-butyl-4-(4-fluorophenyl)-2-phenyl-1H-imidazol-5-yl]methyl}-N-(2,3-dihydro- |
| | 1,4-benzodioxin-6-ylmethyl)butan-1-amine |
| 1497. | N-{[1-butyl-4-(4-chlorophenyl)-2-phenyl-1H-imidazol-5-yl]methyl}-N-(2,3-dihydro- |
| | 1,4-benzodioxin-6-ylmethyl)butan-1-amine |
| 1498. | N-{[1-butyl-4-(4-methoxyphenyl)-2-phenyl-1H-imidazol-5-yl]methyl}-N-(2,3-dihydro- |
| | 1,4-benzodioxin-6-ylmethyl)butan-1-amine |
| 1499. | N-{[1-butyl-4-(2-methoxyphenyl)-2-phenyl-1H-imidazol-5-yl]methyl}-N-(2,3-dihydro- |
| | 1,4-benzodioxin-6-ylmethyl)butan-1-amine |
| 1500. | N-[4-(benzyloxy)benzyl]-N-{[1-butyl-4-(4-chlorophenyl)-2-phenyl-1H-imidazol-5- |
| | yl]methyl}butan-1-amine |
| 1501. | N-[3-(benzyloxy)-4-methoxybenzyl]-N-{[1-butyl-4-(4-chlorophenyl)-2-phenyl-1H- |
| | imidazol-5-yl]methyl}butan-1-amine |
| 1502. | N-{[1-butyl-4-(4-methoxyphenyl)-2-phenyl-1H-imidazol-5-yl]methyl}-N-(3,4- |
| | diethoxybenzyl)butan-1-amine |
| 1503. | N-{[1-butyl-4-(4-chlorophenyl)-2-phenyl-1H-imidazol-5-yl]methyl}-N-(2,3- |
| | dimethoxybenzyl)butan-1-amine |
| 1504. | N-{[1-butyl-4-(4-methoxyphenyl)-2-phenyl-1H-imidazol-5-yl]methyl}-N-(2,3- |
| | dimethoxybenzyl)butan-1-amine |
| 1505. | N-{[1-butyl-4-(4-fluorophenyl)-2-phenyl-1H-imidazol-5-yl]methyl}-N-(3,4- |
| | dimethoxybenzyl)butan-1-amine |
| 1506. | N-{[1-butyl-4-(4-chlorophenyl)-2-phenyl-1H-imidazol-5-yl]methyl}-N-(2- |
| | nitrobenzyl)butan-1-amine |
| 1507. | N-{[1-butyl-4-(4-chlorophenyl)-2-phenyl-1H-imidazol-5-yl]methyl}-N-(3- |
| | nitrobenzyl)butan-1-amine |
| 1508. | N-{[1-butyl-4-(4-chlorophenyl)-2-phenyl-1H-imidazol-5-yl]methyl}-N-(3-methoxy-2- |
| | nitrobenzyl)butan-1-amine |
| 1509. | N-{[1-butyl-4-(4-methoxyphenyl)-2-phenyl-1H-imidazol-5-yl]methyl}-N-(3-methoxy- |
| | 2-nitrobenzyl)butan-1-amine |
| 1510. | N-{[1-butyl-4-(4-chlorophenyl)-2-phenyl-1H-imidazol-5-yl]methyl}-N-(2-chloro-6- |
| | nitrobenzyl)butan-1-amine |
| 1511. | N-{[1-butyl-4-(4-chlorophenyl)-2-phenyl-1H-imidazol-5-yl]methyl}-N-(2-fluoro-5- |
| | nitrobenzyl)butan-1-amine |
| 1512. | N-{[1-butyl-4-(4-chlorophenyl)-2-phenyl-1H-imidazol-5-yl]methyl}-N-(2,5- |
| | difluorobenzyl)butan-1-amine |
| 1513. | N-{[1-butyl-4-(4-chlorophenyl)-2-phenyl-1H-imidazol-5-yl]methyl}-N-(2,3- |
| | dichlorobenzyl)butan-1-amine |
| 1514. | N-[3,5-bis(trifluoromethyl)benzyl]-N-{[1-butyl-4-(4-chlorophenyl)-2-phenyl-1H- |
| | imidazol-5-yl]methyl}butan-1-amine |
| 1515. | N-{[1-butyl-4-(4-chlorophenyl)-2-phenyl-1H-imidazol-5-yl]methyl}-N-(2,6- |
| | dichlorobenzyl)butan-1-amine |
| 1516. | N-{[1-butyl-4-(4-chlorophenyl)-2-phenyl-1H-imidazol-5-yl]methyl}-N-(2-chloro-6- |
| | fluorobenzyl)butan-1-amine |
| 1517. | N-{[1-butyl-4-(4-fluorophenyl)-2-phenyl-1H-imidazol-5-yl]methyl}-N-(2,6- |
| | difluorobenzyl)butan-1-amine |
| 1518. | N-{[1-butyl-4-(4-chlorophenyl)-2-phenyl-1H-imidazol-5-yl]methyl}-N-(2,6- |
| | difluorobenzyl)butan-1-amine |
| 1519. | N-{[1-butyl-4-(4-chlorophenyl)-2-phenyl-1H-imidazol-5-yl]methyl}-N-(2,4- |
| | difluorobenzyl)butan-1-amine |
| 1520. | 2-[(butyl{[1-butyl-4-(4-chlorophenyl)-2-phenyl-1H-imidazol-5- |
| | yl]methyl}amino)methyl]-4-chlorophenol |
| 1521. | 4-bromo-2-[(butyl{[1-butyl-4-(4-chlorophenyl)-2-phenyl-1H-imidazol-5- |
| | yl]methyl}amino)methyl]-6-methoxyphenol |
| 1522. | 2-[(butyl{[1-butyl-4-(4-fluorophenyl)-2-phenyl-1H-imidazol-5- |
| | yl]methyl}amino)methyl]-6-methylphenol |
| 1523. | 2-[(butyl{[1-butyl-4-(4-chlorophenyl)-2-phenyl-1H-imidazol-5- |
| | yl]methyl}amino)methyl]-6-methylphenol |
| 1524. | 2-[(butyl{[1-butyl-4-(4-chlorophenyl)-2-phenyl-1H-imidazol-5- |
| | yl]methyl}amino)methyl]-4-methylphenol |
| 1525. | 4-tert-butyl-2-[(butyl{[1-butyl-4-(4-chlorophenyl)-2-phenyl-1H-imidazol-5- |
| | yl]methyl}amino)methyl]phenol |
| 1526. | 4-[(butyl{[1-butyl-4-(4-fluorophenyl)-2-phenyl-1H-imidazol-5- |
| | yl]methyl}amino)methyl]-2-ethoxyphenol |
| 1527. | 4-[(butyl{[1-butyl-4-(2-methoxyphenyl)-2-phenyl-1H-imidazol-5- |
| | yl]methyl}amino)methyl]-2-ethoxyphenol |
| 1528. | 4-[(butyl{[1-butyl-4-(4-fluorophenyl)-2-phenyl-1H-imidazol-5- |
| | yl]methyl}amino)methyl]-2,6-dimethylphenol |
| 1529. | 4-[(butyl{[1-butyl-4-(4-chlorophenyl)-2-phenyl-1H-imidazol-5- |
| | yl]methyl}amino)methyl]-2,6-dimethylphenol |
| 1530. | 4-[(butyl{[1-butyl-4-(4-methoxyphenyl)-2-phenyl-1H-imidazol-5- |
| | yl]methyl}amino)methyl]-2,6-dimethylphenol |
| 1531. | 4-[(butyl{[1-butyl-4-(2-methoxyphenyl)-2-phenyl-1H-imidazol-5- |
| | yl]methyl}amino)methyl]-2,6-dimethylphenol |
| 1532. | 4-[(butyl{[1-butyl-4-(4-fluorophenyl)-2-phenyl-1H-imidazol-5- |
| | yl]methyl}amino)methyl]-2-methylphenol |
| 1533. | 4-[(butyl{[1-butyl-4-(4-chlorophenyl)-2-phenyl-1H-imidazol-5- |
| | yl]methyl}amino)methyl]-2-methylphenol |
| 1534. | 4-[(butyl{[1-butyl-4-(4-methoxypheyl)-2-phenyl-1H-imidazol-5- |
| | yl]methyl}amino)methyl]-2-methylphenol |
| 1535. | 4-[(butyl{[1-butyl-4-(2-methoxyphenyl)-2-phenyl-1H-imidazol-5- |
| | yl]methyl}amino)methyl]-2-methylphenol |
| 1536. | N-{[1-butyl-4-(4-chlorophenyl)-2-phenyl-1H-imidazol-5-yl]methyl}-N-[(9-ethyl-9H- |
| | carbazol-3-yl)methyl]butan-1-amine |
| 1537. | 3-[(butyl{[1-butyl-4-(4-fluorophenyl)-2-phenyl-1H-imidazol-5- |
| | yl]methyl}amino)methyl]phenol |
| 1538. | 3-[(butyl{[1-butyl-4-(4-chlorophenyl)-2-phenyl-1H-imidazol-5- |
| | yl]methyl}amino)methyl]phenol |
| 1539. | 3-[(butyl{[1-butyl-4-(4-methoxyphenyl)-2-phenyl-1H-imidazol-5- |
| | yl]methyl}amino)methyl]phenol |
| 1540. | 3-[(butyl{[1-butyl-4-(2-methoxyphenyl)-2-phenyl-1H-imidazol-5- |
| | yl]methyl}amino)methyl]phenol |
| 1541. | 5-[(butyl{[1-butyl-4-(4-fluorophenyl)-2-phenyl-1H-imidazol-5- |
| | yl]methyl}amino)methyl]-2-methoxyphenol |
| 1542. | 5-[(butyl{[1-butyl-4-(2-methoxyphenyl)-2-phenyl-1H-imidazol-5- |
| | yl]methyl}amino)methyl]-2-methoxyphenol |
| 1543. | 4-[(butyl{[1-butyl-4-(4-fluorophenyl)-2-phenyl-1H-imidazol-5- |
| | yl]methyl}amino)methyl]phenol |
| 1544. | 4-[(butyl{[1-butyl-4-(4-methoxyphenyl)-2-phenyl-1H-imidazol-5- |
| | yl]methyl}amino)methyl]phenol |
| 1545. | 4-[(butyl{[1-butyl-4-(2-methoxyphenyl)-2-phenyl-1H-imidazol-5- |
| | yl]methyl}amino)methyl]phenol |
| 1546. | 4-[(butyl{[1-butyl-4-(4-fluorophenyl)-2-phenyl-1H-imidazol-5- |
| | yl]methyl}amino)methyl]-2-methoxyphenol |
| 1547. | 4-[(butyl{[1-butyl-4-(2-methoxyphenyl)-2-phenyl-1H-imidazol-5- |
| | yl]methyl}amino)methyl]-2-methoxyphenol |
| 1548. | 4-[(butyl{[1-butyl-4-(4-fluorophenyl)-2-phenyl-1H-imidazol-5- |
| | yl]methyl}amino)methyl]-3-chlorophenol |
| 1549. | 4-[(butyl{[1-butyl-4-(4-chlorophenyl)-2-phenyl-1H-imidazol-5- |
| | yl]methyl}amino)methyl]-3-chlorophenol |
| 1550. | 4-[(butyl{[1-butyl-4-(2-methoxyphenyl)-2-phenyl-1H-imidazol-5- |
| | yl]methyl}amino)methyl]-3-chlorophenol |
| 1551. | N-{[1-butyl-4-(4-methoxyphenyl)-2-phenyl-1H-imidazol-5-yl]methyl}-N-(2,3,4- |
| | trimethoxybenzyl)butan-1-amine |
| 1552. | N-(1,3-benzodioxol-4-ylmethyl)-N-{[1-butyl-4-(4-chlorophenyl)-2-phenyl-1H- |
| | imidazol-5-yl]methyl}butan-1-amine |
| 1553. | N-{[1-butyl-4-(4-chlorophenyl)-2-phenyl-1H-imidazol-5-yl]methyl}-N-(4-methoxy-3- |
| | methylbenzyl)butan-1-amine |
| 1554. | N-{[1-butyl-4-(4-methoxyphenyl)-2-phenyl-1H-imidazol-5-yl]methyl}-N-(4-methoxy- |
| | 3-methylbenzyl)butan-1-amine |
| 1555. | N-{[1-butyl-4-(4-fluorophenyl)-2-phenyl-1H-imidazol-5-yl]methyl}-N-(2,4-dimethoxy- |
| | 3-methylbenzyl)butan-1-amine |
| 1556. | N-{[1-butyl-4-(4-chlorophenyl)-2-phenyl-1H-imidazol-5-yl]methyl}-N-(2,4- |
| | dimethoxy-3-methylbenzyl)butan-1-amine |
| 1557. | N-{[1-butyl-4-(4-methoxyphenyl)-2-phenyl-1H-imidazol-5-yl]methyl}-N-(2,4- |
| | dimethoxy-3-methylbenzyl)butan-1-amine |
| 1558. | N-{[1-butyl-4-(2-methoxyphenyl)-2-phenyl-1H-imidazol-5-yl]methyl}-N-(2,4- |
| | dimethoxy-3-methylbenzyl)butan-1-amine |
| 1559. | N-{[1-butyl-4-(4-chlorophenyl)-2-phenyl-1H-imidazol-5-yl]methyl}-N-(2,3,4- |
| | trifluorobenzyl)butan-1-amine |
| 1560. | N-{[1-butyl-4-(4-chlorophenyl)-2-phenyl-1H-imidazol-5-yl]methyl}-N-[2-fluoro-6- |
| | (trifluoromethyl)benzyl]butan-1-amine |
| 1561. | N-{[1-butyl-4-(4-chlorophenyl)-2-phenyl-1H-imidazol-5-yl]methyl}-N-(2-chloro-3,4- |
| | dimethoxybenzyl)butan-1-amine |
| 1562. | N-{[1-butyl-4-(4-chlorophenyl)-2-phenyl-1H-imidazol-5-yl]methyl}-N-(2-chloro-4- |
| | fluorobenzyl)butan-1-amine |
| 1563. | N-{[1-butyl-4-(4-fluorophenyl)-2-phenyl-1H-imidazol-5-yl]methyl}-N-[4- |
| | (difluoromethoxy)benzyl]butan-1-amine |
| 1564. | N-{[1-butyl-4-(4-chlorophenyl)-2-phenyl-1H-imidazol-5-yl]methyl}-N-[4- |
| | (difluoromethoxy)benzyl]butan-1-amine |
| 1565. | N-{[1-butyl-4-(2-methoxyphenyl)-2-phenyl-1H-imidazol-5-yl]methyl}-N-[4- |
| | (difluoromethoxy)benzyl]butan-1-amine |
| 1566. | N-{[1-butyl-4-(4-chlorophenyl)-2-phenyl-1H-imidazol-5-yl]methyl}-N-[4- |
| | (trifluoromethoxy)benzyl]butan-1-amine |
| 1567. | N-{[1-butyl-4-(4-chlorophenyl)-2-phenyl-1H-imidazol-5-yl]methyl}-N-[4-fluoro-3- |
| | (trifluoromethyl)benzyl]butan-1-amine |
| 1568. | N-(3-bromo-4-fluorobenzyl)-N-{[1-butyl-4-(4-chlorophenyl)-2-phenyl-1H-imidazol-5- |
| | yl]methyl}butan-1-amine |
| 1569. | N-(3-bromo-4-methoxybenzyl)-N-{[1-butyl-4-(4-chlorophenyl)-2-phenyl-1H-imidazol- |
| | 5-yl]methyl}butan-1-amine |
| 1570. | N-(3-bromo-4-methoxybenzyl)-N-{[1-butyl-4-(4-methoxyphenyl)-2-phenyl-1H- |
| | imidazol-5-yl]methyl}butan-1-amine |
| 1571. | N-{[1-butyl-4-(4-fluorophenyl)-2-phenyl-1H-imidazol-5-yl]methyl}-N-(4- |
| | propoxybenzyl)butan-1-amine |
| 1572. | N-{[1-butyl-4-(4-chlorophenyl)-2-phenyl-1H-imidazol-5-yl]methyl}-N-(4- |
| | propoxybenzyl)butan-1-amine |
| 1573. | N-{[1-butyl-4-(4-methoxyphenyl)-2-phenyl-1H-imidazol-5-yl]methyl}-N-(4- |
| | propoxybenzyl)butan-1-amine |
| 1574. | N-(4-tert-butylbenzyl)-N-{[1-butyl-4-(4-chlorophenyl)-2-phenyl-1H-imidazol-5- |
| | yl]methyl}butan-1-amine |
| 1575. | N-(4-tert-butylbenzyl)-N-{[1-butyl-4-(4-methoxyphenyl)-2-phenyl-1H-imidazol-5- |
| | yl]methyl}butan-1-amine |
| 1576. | N-{[1-butyl-4-(4-chlorophenyl)-2-phenyl-1H-imidazol-5-yl]methyl}-N-(3-fluoro-4- |
| | methoxybenzyl)butan-1-amine |
| 1577. | 1-(1,3-benzodioxol-5-yl)-N-(1,3-benzodioxol-5-ylmethyl)-N-[(5-methyl-1,3-diphenyl- |
| | 1H-pyrazol-4-yl)methyl]methanamine |
| 1578. | 1-(1,3-benzodioxol-5-yl)-N-(cyclopentylmethyl)-N-[(5-methyl-1,3-diphenyl-1H- |
| | pyrazol-4-yl)methyl]methanamine |
| 1579. | 4-({(1,3-benzodioxol-5-ylmethyl)[(1,3-diphenyl-5-propyl-1H-pyrazol-4- |
| | yl)methyl]amino}methyl)-3-chlorophenol |
| 1580. | 1-(1,3-benzodioxol-5-yl)-N-benzyl-N-[(1,3-diphenyl-5-propyl-1H-pyrazol-4- |
| | yl)methyl]methanamine |
| 1581. | 1-(1,3-benzodioxol-5-yl)-N-(1,3-benzodioxol-5-ylmethyl)-N-[(1,3-diphenyl-5-propyl- |
| | 1H-pyrazol-4-yl)methyl]methanamine |
| 1582. | N-(1,3-benzodioxol-5-ylmethyl)-N-[(1,3-diphenyl-5-propyl-1H-pyrazol-4- |
| | yl)methyl]butan-1-amine |
| 1583. | 4-({benzyl[(1,3-diphenyl-5-propyl-1H-pyrazol-4-yl)methyl]amino}methyl)-3- |
| | chlorophenol |
| 1584. | 4-({butyl[(1,3-diphenyl-5-propyl-1H-pyrazol-4-yl)methyl]amino}methyl)-3- |
| | chlorophenol |
| 1585. | N-(2,3-dihydro-1H-indan-2-yl)-N-(3-methylbenzyl)-1,1′-biphenyl-2-carboxamide |
| 1586. | N-(2,3-dihydro-1H-indan-2-yl)-N-(2-methylbenzyl)-1,1′-biphenyl-2-carboxamide |
| 1587. | N-(2,3-dihydro-1H-indan-2-yl)-N-(4-methylbenzyl)-1,1′-biphenyl-2-carboxamide |
| 1588. | N-(2,3-dihydro-1H-indan-2-yl)-N-(2-fluorobenzyl)-1,1′-biphenyl-2-carboxamide |
| 1589. | N-(2,3-dihydro-1H-indan-2-yl)-3-methyl-N-(4-methylbenzyl)-2-(2- |
| | methylphenyl)butanamide |
| 1590. | 2-cyclopentyl-N-(2,3-dihydro-1H-indan-2-yl)-N-(4-methylbenzyl)-2-phenylacetamide |
| 1591. | N-(4-chlorobenzyl)-N-(2,3-dihydro-1H-indan-2-yl)-1,1′-biphenyl-2-carboxamide |
| 1592. | N-benzyl-N-(1,2,3,4-tetrahydronaphthalen-2-yl)-1,1′-biphenyl-2-carboxamide |
| 1593. | N-(2,3-dihydro-1H-indan-2-yl)-N-(2-phenylethyl)-1,1′-biphenyl-2-carboxamide |
| 1594. | N-(2,3-dihydro-1H-indan-2-yl)-N-(3-phenylpropyl)-1,1′-biphenyl-2-carboxamide |
| 1595. | N-(2,3-dihydro-1H-indan-2-yl)-8-iodo-N-(2-methylbenzyl)-1-naphthamide |
| 1596. | N-(2,3-dihydro-1H-indan-2-yl)-N-(3-methylbenzyl)-9H-fluorene-4-carboxamide |
| 1597. | N-(2,3-dihydro-1H-indan-2-yl)-N-(2-methylbenzyl)-9H-fluorene-4-carboxamide |
| 1598. | N-(2,3-dihydro-1H-indan-2-yl)-N-(2-fluorobenzyl)-9H-fluorene-4-carboxamide |
| 1599. | 8-bromo-N-(2,3-dihydro-1H-indan-2-yl)-N-(2-methylbenzyl)-1-naphthamide |
| 1600. | 8-bromo-N-[(6-chloro-1-cyclopentyl-1H-benzimidazol-2-yl)methyl]-N-cyclopentyl-1- |
| | naphthamide |
| 1601. | N-(2-cyclohexylethyl)-N-(2,3-dihydro-1H-indan-2-yl)-2-(methylthio)benzamide |
| 1602. | N-(2-cyclohexylethyl)-N-(2,3-dihydro-1H-indan-2-yl)-2,6-dimethoxybenzamide |
| 1603. | 3,5-dichloro-N-(2-cyclohexylethyl)-2,6-dimethoxy-N-(3-phenylpropyl)benzamide |
| 1604. | N-(2-cyclohexylethyl)-N-(2,3-dihydro-1H-indan-2-yl)-2-fluoro-6-iodobenzamide |
| 1605. | N-(2-cyclohexylethyl)-8-iodo-N-(2-phenylpropyl)-1-naphthamide |
| 1606. | N-(2-cyclopentylethyl)-N-(2-phenylethyl)-9H-fluorene-4-carboxamide |
| 1607. | N-(2-cyclohexylethyl)-N-(2-phenylpropyl)-9H-fluorene-4-carboxamide |
| 1608. | N-(2,3-dihydro-1H-indan-2-yl)-N-(2-methylbenzyl)-2-phenoxybenzamide |
| 1609. | 2-butoxy-N-(2,3-dihydro-1H-indan-2-yl)-N-(3-methylbenzyl)benzamide |
| 1610. | 2-butoxy-N-(2,3-dihydro-1H-indan-2-yl)-N-(2-methylbenzyl)benzamide |
| 1611. | N-benzyl-2-butoxy-N-(1,2,3,4-tetrahydronaphthalen-2-yl)benzamide |
| 1612. | N-(2,3-dihydro-1H-indan-2-yl)-2,3-dimethoxy-N-(2-methylbenzyl)benzamide |
| 1613. | N-(2,3-dihydro-1H-indan-2-yl)-2,3-dimethoxy-N-(4-methylbenzyl)benzamide |
| 1614. | N-(4-chlorobenzyl)-N-(2,3-dihydro-1H-indan-2-yl)-2,3-dimethoxybenzamide |
| 1615. | N-benzyl-N-(2,3-dihydro-1H-indan-2-yl)-2-(trifluoromethoxy)benzamide |
| 1616. | 1-(2-cyclohexylethyl)-2-(3,5-dimethoxybenzoyl)-1,2,3,4-tetrahydroisoquinoline |
| 1617. | 1-(2-cyclohexylethyl)-2-(2,3-dimethoxybenzoyl)-1,2,3,4-tetrahydroisoquinoline |
| 1618. | 1-(2-cyclohexylethyl)-2-[2-(trifluoromethyl)benzoyl]-1,2,3,4-tetrahydroisoquinoline |
| 1619. | 2-[1-(2-chlorophenyl)-3,4-dihydroisoquinolin-2(1H)-yl]-N-(2-methylbenzyl)-N-(2- |
| | phenylethyl)acetamide |
| 1620. | N-(2-chlorobenzyl)-2-[1-(2-methoxyphenyl)-3,4-dihydroisoquinolin-2(1H)-yl]-N-(2- |
| | phenylethyl)acetamide |
| 1621. | N-(2-chlorobenzyl)-2-[1-(2-chlorophenyl)-3,4-dihydroisoquinolin-2(1H)-yl]-N-(2- |
| | phenylethyl)acetamide |
| 1622. | N-(2-chlorobenzyl)-N-(2-phenylethyl)-2-[1-[2-(trifluoromethyl)phenyl]-3,4- |
| | dihydroisoquinolin-2(1H)-yl]acetamide |
| 1623. | 1-(2-chlorophenyl)-2-[2-oxo-2-(2-phenylazepan-1-yl)ethyl]-1,2,3,4- |
| | tetrahydroisoquinoline |
| 1624. | 2-[2-oxo-2-(2-phenylazepan-1-yl)ethyl]-1-[2-(trifluoromethyl)phenyl]-1,2,3,4- |
| | tetrahydroisoquinoline |
| 1625. | N-{[1-(3-fluorobenzyl)-1H-benzimidazol-2-yl]methyl}-N-pentyl-2-(1H-pyrrol-1- |
| | yl)benzamide |
| 1626. | N-(4-fluorobenzyl)-1,2,5-trimethyl-N-[(3-propyl-3H-imidazo[4,5-b]pyridin-2- |
| | yl)methyl]-1H-pyrrole-3-carboxamide |
| 1627. | N-benzyl-N-[(3-propyl-3H-imidazo[4,5-b]pyridin-2-yl)methyl]-2-(1H-pyrrol-1- |
| | yl)benzamide |
| 1628. | N-(2-fluorobenzyl)-N-[(3-propyl-3H-imidazo[4,5-b]pyridin-2-yl)methyl]-2-(1H-pyrrol- |
| | 1-yl)benzamide |
| 1629. | N-(4-fluorobenzyl)-N-[(3-propyl-3H-imidazo[4,5-b]pyridin-2-yl)methyl]-2-(1H-pyrrol- |
| | 1-yl)benzamide |
| 1630. | N-(4-methoxybenzyl)-N-[(3-propyl-3H-imidazo[4,5-b]pyridin-2-yl)methyl]-2-(1H- |
| | pyrrol-1-yl)benzamide |
| 1631. | N-(3-cyclohexyl-1-phenylpropyl)-N-methyl-2-(1H-pyrrol-1-yl)benzamide |
| 1632. | 1-(2-cyclohexylethyl)-2-[2-(1H-pyrrol-1-yl)benzoyl]-1,2,3,4-tetrahydroisoquinoline |
| 1633. | N-benzyl-N-pentyl-2-(1H-pyrrol-1-yl)benzamide |
| 1634. | N,N-dibenzyl-2-(1H-pyrrol-1-yl)benzamide |
| 1635. | 5-tert-butyl-N-{[1-(2,6-dimethoxybenzyl)-1H-benzimidazol-2-yl]methyl}-2-methyl-N- |
| | pentyl-3-furamide |
| 1636. | N-butyl-N-{[1-(2,6-dimethoxybenzyl)-1H-benzimidazol-2-yl]methyl}-4- |
| | (methylthio)benzamide |
| 1637. | N-{[1-(2,6-dimethoxybenzyl)-1H-benzimidazol-2-yl]methyl}-4-(methylthio)-N- |
| | pentylbenzamide |
| 1638. | N-{[1-(2,6-dimethoxybenzyl)-1H-benzimidazol-2-yl]methyl}-3-fluoro-4-methoxy-N- |
| | pentylbenzamide |
| 1639. | N-butyl-4-chloro-N-{[1-(2,6-dimethoxybenzyl)-1H-benzimidazol-2-yl]methyl}-3- |
| | methylbenzamide |
| 1640. | N-{[1-(2,6-dimethoxybenzyl)-1H-benzimidazol-2-yl]methyl}-N-pentyl-4- |
| | propoxybenzamide |
| 1641. | N-{[1-(2,6-dimethoxybenzyl)-1H-benzimidazol-2-yl]methyl}-4-isopropoxy-N- |
| | pentylbenzamide |
| 1642. | N-{[1-(2,6-dimethoxybenzyl)-1H-benzimidazol-2-yl]methyl}-3,4-dimethoxy-N- |
| | pentylbenzamide |
| 1643. | N-{[1-(2,6-dimethoxybenzyl)-1H-benzimidazol-2-yl]methyl}-N-isopentyl-3,4- |
| | dimethoxybenzamide |
| 1644. | N-butyl-N-{[3-(2-chlorobenzyl)-3H-imidazo[4,5-b]pyridin-2-yl]methyl}-9H-fluorene- |
| | 4-carboxamide |
| 1645. | N-{[3-(2-chlorobenzyl)-3H-imidazo[4,5-b]pyridin-2-yl]methyl}-N-isobutyl-9H- |
| | fluorene-4-carboxamide |
| 1646. | N-{[3-(2-chlorobenzyl)-3H-imidazo[4,5-b]pyridin-2-yl]methyl}-N-pentyl-9H-fluorene- |
| | 4-carboxamide |
| 1647. | 2-chloro-N-{[3-(2-chlorobenzyl)-3H-imidazo[4,5-b]pyridin-2-yl]methyl}-3,4- |
| | dimethoxy-N-pentylbenzamide |
| 1648. | 8-bromo-N-butyl-N-{[3-(2-chlorobenzyl)-3H-imidazo[4,5-b]pyridin-2-yl]methyl}-1- |
| | naphthamide |
| 1649. | 8-bromo-N-{[3-(2-chlorobenzyl)-3H-imidazo[4,5-b]pyridin-2-yl]methyl}-N-isobutyl-1- |
| | naphthamide |
| 1650. | 8-bromo-N-{[3-(2-chlorobenzyl)-3H-imidazo[4,5-b]pyridin-2-yl]methyl}-N-pentyl-1- |
| | naphthamide |
| 1651. | N-butyl-N-{[3-(2-chlorobenzyl)-3H-imidazo[4,5-b]pyridin-2-yl]methyl}-2-(1H-pyrrol- |
| | 1-yl)benzamide |
| 1652. | N-{[3-(2-chlorobenzyl)-3H-imidazo[4,5-b]pyridin-2-yl]methyl}-N-isobutyl-2-(1H- |
| | pyrrol-1-yl)benzamide |
| 1653. | N-{[3-(2-chlorobenzyl)-3H-imidazo[4,5-b]pyridin-2-yl]methyl}-N-pentyl-2-(1H- |
| | pyrrol-1-yl)benzamide |
| 1654. | N-{[3-(2-chlorobenzyl)-3H-imidazo[4,5-b]pyridin-2-yl]methyl}-N-isopentyl-2-(1H- |
| | pyrrol-1-yl)benzamide |
| 1655. | N-butyl-N-{[3-(3-chlorobenzyl)-3H-imidazo[4,5-b]pyridin-2-yl]methyl}-9H-fluorene- |
| | 4-carboxamide |
| 1656. | N-butyl-N-{[3-(3-chlorobenzyl)-3H-imidazo[4,5-b]pyridin-2-yl]methyl}-2-(1H-pyrrol- |
| | 1-yl)benzamide |
| 1657. | N-{[3-(3-chlorobenzyl)-3H-imidazo[4,5-b]pyridin-2-yl]methyl}-N-isobutyl-2-(1H- |
| | pyrrol-1-yl)benzamide |
| 1658. | N-{[3-(3-chlorobenzyl)-3H-imidazo[4,5-b]pyridin-2-yl]methyl}-N-pentyl-2-(1H- |
| | pyrrol-1-yl)benzamide |
| 1659. | N-{[3-(3-chlorobenzyl)-3H-imidazo[4,5-b]pyridin-2-yl]methyl}-N-isopentyl-2-(1H- |
| | pyrrol-1-yl)benzamide |
| 1660. | N-(2,3-dihydro-1H-indan-2-yl)-N-(2-fluorobenzyl)-2-[1-(3-methylphenyl)-3,4- |
| | dihydroisoquinolin-2(1H)-yl]acetamide |
| 1661. | N-(2,3-dihydro-1H-indan-2-yl)-N-(2-fluorobenzyl)-2-[1-(4-methylphenyl)-3,4- |
| | dihydroisoquinolin-2(1H)-yl]acetamide |
| 1662. | N-(2,3-dihydro-1H-indan-2-yl)-N-(2-fluorobenzyl)-2-[1-(4-fluorophenyl)-3,4- |
| | dihydroisoquinolin-2(1H)-yl]acetamide |
| 1663. | N-(2,3-dihydro-1H-indan-2-yl)-N-(2-fluorobenzyl)-2-[1-(4-methoxyphenyl)-3,4- |
| | dihydroisoquinolin-2(1H)-yl]acetamide |
| 1664. | 2-[1-(3-chlorophenyl)-3,4-dihydroisoquinolin-2(1H)-yl]-N-(2,3-dihydro-1H-indan-2- |
| | yl)-N-(2-fluorobenzyl)acetamide |
| 1665. | N-benzyl-N-(2,3-dihydro-1H-indan-2-yl)-2-[1-(3-methylphenyl)-3,4- |
| | dihydroisoquinolin-2(1H)-yl]acetamide |
| 1666. | N-benzyl-N-(2,3-dihydro-1H-indan-2-yl)-2-[1-(4-methylphenyl)-3,4- |
| | dihydroisoquinolin-2(1H)-yl]acetamide |
| 1667. | N-benzyl-N-(2,3-dihydro-1H-indan-2-yl)-2-[1-(4-methoxyphenyl)-3,4- |
| | dihydroisoquinolin-2(1H)-yl]acetamide |
| 1668. | N-[(5-bromo-2-phenyl-1,3-thiazol-4-yl)methyl]-N-isopentyl-2-(1H-pyrrol-1- |
| | yl)benzamide |
| 1669. | N-butyl-N-[(5-chloro-2-phenyl-1,3-thiazol-4-yl)methyl]-2-(1H-pyrrol-1-yl)benzamide |
| 1670. | N-[(5-chloro-2-phenyl-1,3-thiazol-4-yl)methyl]-N-isobutyl-2-(1H-pyrrol-1- |
| | yl)benzamide |
| 1671. | N-[(5-chloro-2-phenyl-1,3-thiazol-4-yl)methyl]-N-isopentyl-2-(1H-pyrrol-1- |
| | yl)benzamide |
| 1672. | N-[(5-chloro-2-phenyl-1,3-thiazol-4-yl)methyl]-N-cyclopentyl-2-(1H-pyrrol-1- |
| | yl)benzamide |
| 1673. | 8-bromo-N-[(2-phenylquinolin-4-yl)methyl]-N-propyl-1-naphthamide |
| 1674. | N-(2-chlorobenzyl)-N-(2-phenylethyl)-9H-fluorene-4-carboxamide |
| 1675. | 2-(2-iodophenyl)-N-[(2-phenylquinolin-4-yl)methyl]-N-propylacetamide |
| 1676. | 2-(2-phenoxyphenyl)-N-[(2-phenylquinolin-4-yl)methyl]-N-propylacetamide |
| 1677. | 2-(2-methoxyphenyl)-N-[(2-phenylquinolin-4-yl)methyl]-N-propylacetamide |
| 1678. | 2-(2-bromophenyl)-N-[(2-phenylquinolin-4-yl)methyl]-N-propylacetamide |
| 1679. | N-benzyl-N-[2-(2-chlorophenyl)ethyl]-9H-fluorene-4-carboxamide |
| 1680. | N-benzyl-N-(3-phenylbutyl)-9H-fluorene-4-carboxamide |
| 1681. | N-benzyl-N-[2-(4-chlorophenyl)ethyl]-9H-fluorene-4-carboxamide |
| 1682. | N-benzyl-N-(2,3-dihydro-1H-indan-2-yl)-1,1′-biphenyl-2-carboxamide |
| 1683. | N-benzyl-N-(2,3-dihydro-1H-indan-2-yl)-9H-fluorene-4-carboxamide |
| 1684. | N-benzyl-N-[2-(4-fluorophenyl)ethyl]-9H-fluorene-4-carboxamide |
| 1685. | N-benzyl-N-[2-(4-methoxyphenyl)ethyl]-9H-fluorene-4-carboxamide |
| 1686. | N-benzyl-N-[2-(4-methoxyphenyl)-1-methylethyl]-9H-fluorene-4-carboxamide |
| 1687. | 2-chloro-N-{[1-(2,3-dihydro-1H-indan-2-yl)-2-phenyl-1H-imidazol-5-yl]methyl}-N-(4- |
| | methoxybenzyl)benzamide |
| 1688. | N-{[1-(2,3-dihydro-1H-indan-2-yl)-2-phenyl-1H-imidazol-5-yl]methyl}-2,5-difluoro- |
| | N-(4-methoxybenzyl)benzamide |
| 1689. | N-(cyclohexylmethyl)-N-{[1-(2,3-dihydro-1H-indan-2-yl)-2-phenyl-1H-imidazol-5- |
| | yl]methyl}-2,5-difluorobenzamide |
| 1690. | 5-chloro-N-{[1-(2,3-dihydro-1H-indan-2-yl)-2-phenyl-1H-imidazol-5-yl]methyl}-2- |
| | methoxy-N-(4-methoxybenzyl)benzamide |
| 1691. | 5-chloro-N-(cyclohexylmethyl)-N-{[1-(2,3-dihydro-1H-indan-2-yl)-2-phenyl-1H- |
| | imidazol-5-yl]methyl}-2-methoxybenzamide |
| 1692. | N-{[1-(2,3-dihydro-1H-indan-2-yl)-2-phenyl-1H-imidazol-5-yl]methyl}-N-(4- |
| | methoxybenzyl)-2-(trifluoromethyl)benzamide |
| 1693. | N-benzyl-2,5-dichloro-N-{[1-(2,3-dihydro-1H-indan-2-yl)-2-phenyl-1H-imidazol-5- |
| | yl]methyl}benzamide |
| 1694. | 2,5-dichloro-N-{[1-(2,3-dihydro-1H-indan-2-yl)-2-phenyl-1H-imidazol-5-yl]methyl}- |
| | N-(4-methoxybenzyl)benzamide |
| 1695. | 2,5-dichloro-N-(cyclohexylmethyl)-N-{[1-(2,3-dihydro-1H-indan-2-yl)-2-phenyl-1H- |
| | imidazol-5-yl]methyl}benzamide |
| 1696. | 2-bromo-N-{[1-(2,3-dihydro-1H-indan-2-yl)-2-phenyl-1H-imidazol-5-yl]methyl}-N-(4- |
| | methoxybenzyl)benzamide |
| 1697. | N-{[1-(2,3-dihydro-1H-indan-2-yl)-2-phenyl-1H-imidazol-5-yl]methyl}-N-(4- |
| | methoxybenzyl)-2-(2-phenylethyl)benzamide |
| 1698. | N-{[1-(2,3-dihydro-1H-indan-2-yl)-2-phenyl-1H-imidazol-5-yl]methyl}-2-iodo-N-(4- |
| | methoxybenzyl)benzamide |
| 1699. | 3-chloro-N-{[1-(2,3-dihydro-1H-indan-2-yl)-2-phenyl-1H-imidazol-5-yl]methyl}-2,6- |
| | dimethoxy-N-(4-methoxybenzyl)benzamide |
| 1700. | 2-bromo-N-{[1-(2,3-dihydro-1H-indan-2-yl)-2-phenyl-1H-imidazol-5-yl]methyl}-5- |
| | methoxy-N-(4-methoxybenzyl)benzamide |
| 1701. | 3-bromo-N-{[1-(2,3-dihydro-1H-indan-2-yl)-2-phenyl-1H-imidazol-5-yl]methyl}-2,6- |
| | dimethoxy-N-(4-methoxybenzyl)benzamide |
| 1702. | N-{[1-(2,3-dihydro-1H-indan-2-yl)-2-phenyl-1H-imidazol-5-yl]methyl}-2-fluoro-6- |
| | iodo-N-(4-methoxybenzyl)benzamide |
| 1703. | 2-bromo-N-{[1-(2,3-dihydro-1H-indan-2-yl)-2-phenyl-1H-imidazol-5-yl]methyl}-N-(4- |
| | methoxybenzyl-5-methylbenzamide |
| 1704. | 2-bromo-N-(cyclohexylmethyl)-N-{[1-(2,3-dihydro-1H-indan-2-yl)-2-phenyl-1H- |
| | imidazol-5-yl]methyl}-5-methylbenzamide |
| 1705. | 2-chloro-N-{[1-(2,3-dihydro-1H-indan-2-yl)-2-phenyl-1H-imidazol-5-yl]methyl}-N-(4- |
| | methoxybenzyl)-5-(methylthio)benzamide |
| 1706. | 2-chloro-N-{[1-(2,3-dihydro-1H-indan-2-yl)-2-phenyl-1H-imidazol-5-yl]methyl}-N-(4- |
| | methoxybenzyl)-5-(trifluoromethyl)benzamide |
| 1707. | 2-chloro-N-(cyclohexylmethyl)-N-{[1-(2,3-dihydro-1H-indan-2-yl)-2-phenyl-1H- |
| | imidazol-5-yl]methyl}-5-(trifluoromethyl)benzamide |
| 1708. | N-{[1-(2,3-dihydro-1H-indan-2-yl)-2-phenyl-1H-imidazol-5-yl]methyl}-N-(4- |
| | methoxybenzyl)-2-(1H-pyrrol-1-yl)benzamide |
| 1709. | 1-(cyclopentylmethyl)-2-[(2-phenylpiperidin-1-yl)carbonyl]-1,2,3,4- |
| | tetrahydroisoquinoline |
| 1710. | 1-(cyclopentylmethyl)-2-[(2-phenylazepan-1-yl)carbonyl]-1,2,3,4- |
| | tetrahydroisoquinoline |
| 1711. | 1-hexyl-2-[(2-phenylpiperidin-1-yl)carbonyl]-1,2,3,4-tetrahydroisoquinoline |
| 1712. | 1-hexyl-2-[(2-phenylazepan-1-yl)carbonyl]-1,2,3,4-tetrahydroisoquinoline |
| 1713. | N-benzyl-N-(2,3-dihydro-1H-indan-2-yl)-5′-isopropyl-2′-methoxy-1,1′-biphenyl-2- |
| | carboxamide |
| 1714. | N-benzyl-N-(1-naphthylmethyl)-1,1′-biphenyl-2-carboxamide |
| 1715. | N-(1-benzothien-5-ylmethyl)-N-benzyl-1,1′-biphenyl-2-carboxamide |
| 1716. | N-benzyl-2′-fluoro-N-(1-naphthylmethyl)-1,1′-biphenyl-2-carboxamide |
| 1717. | N-(4-chlorobenzyl)-N-(2,3-dihydro-1H-indan-2-yl)-2′-fluoro-1,1′-biphenyl-2- |
| | carboxamide |
| 1718. | N-benzyl-N-(1-naphthylmethyl)-2-thien-3-ylbenzamide |
| 1719. | N-(4-chlorobenzyl)-N-(2,3-dihydro-1H-indan-2-yl)-2-thien-3-ylbenzamide |
| 1720. | N-benzyl-2-(5-chlorothien-2-yl)-N-(2,3-dihydro-1H-indan-2-yl)benzamide |
| 1721. | N-(4-chlorobenzyl)-2-(5-chlorothien-2-yl)-N-(2,3-dihydro-1H-indan-2-yl)benzamide |
| 1722. | N-benzyl-N-(2,3-dihydro-1H-indan-2-yl)-2-(5-methylthien-2-yl)benzamide |
| 1723. | N-(2,3-dihydro-1H-indan-2-yl)-N-(2-methoxybenzyl)-1,1′-biphenyl-2-carboxamide |
| 1724. | N-(2,3-dihydro-1H-indan-2-yl)-N-(3-methoxybenzyl)-1,1′-biphenyl-2-carboxamide |
| 1725. | N-(2,3-dihydro-1H-indan-2-yl)-N-(4-methoxybenzyl)-1,1′-biphenyl-2-carboxamide |
| 1726. | N-(2,3-dihydro-1H-indan-2-yl)-N-(3-hydroxybenzyl)-1,1′-biphenyl-2-carboxamide |
| 1727. | ethyl (3-{[(1,1′-biphenyl-2-ylcarbonyl)(2,3-dihydro-1H-indan-2- |
| | yl)amino]methyl}phenoxy)acetate |
| 1728. | N-(3-methoxybenzyl)-N-(2-phenylethyl)-1,1′-biphenyl-2-carboxamide |
| 1729. | N-(2,3-dihydro-1H-indan-2-yl)-N-(2-hydroxybenzyl)-1,1′-biphenyl-2-carboxamide |
| 1730. | ethyl (2-{[(1,1′-biphenyl-2-ylcarbonyl)(2,3-dihydro-1H-indan-2- |
| | yl)amino]methyl}phenoxy)acetate |
| 1731. | N-(2,3-dihydro-1H-indan-2-yl)-N-(4-hydroxybenzyl)-1,1′-biphenyl-2-carboxamide |
| 1732. | ethyl (4-{[(1,1′-biphenyl-2-ylcarbonyl)(2,3-dihydro-1H-indan-2- |
| | yl)amino]methyl}phenoxy)acetate |
| 1733. | N-(4-methoxybenzyl)-N-[(3 -propyl-3H-imidazo[4,5-b]pyridin-2-yl)methyl]-1,1′- |
| | biphenyl-2-carboxamide |
| 1734. | N-(4-fluorobenzyl)-N-[(3-propyl-3H-imidazo[4,5-b]pyridin-2-yl)methyl]-1,1′-biphenyl- |
| | 2-carboxamide |
| 1735. | N-(2,4-difluorobenzyl)-N-[(3-propyl-3H-imidazo[4,5-b]pyridin-2-yl)methyl]-2-(1H- |
| | pyrrol-1-yl)benzamide |
| 1736. | N-benzyl-N-(2,3-dihydro-1H-indan-2-yl)-2-[4-(trifluoromethyl)phenyl]nicotinamide |
| 1737. | N-benzyl-N-[(1-butyl-1H-benzimidazol-2-yl)methyl]-1-naphthamide |
| 1738. | N˜2˜-(1,3-benzodioxol-5-ylmethyl)-N˜1˜,N˜2˜-dibenzyl-N˜1˜-butylglycinamide |
| 1739. | N˜1˜,N˜2˜-dibenzyl-N˜1˜-butyl-N˜2˜-(2-naphthylmethyl)glycinamide |
| 1740. | 1-(1,3-benzodioxol-5-yl)-N-[(3-butyl-2-phenylquinolin-4-yl)methyl]methanamine |
| 1741. | 1-(1,3-benzodioxol-5-yl)-N-(1,3-benzodioxol-5-ylmethyl)-N-[(3-butyl-2- |
| | phenylquinolin-4-yl)methyl]methanamine |
| 1742. | 1-(1,3-benzodioxol-5-yl)-N-benzyl-N-[(3-butyl-2-phenylquinolin-4- |
| | yl)methyl]methanamine |
| 1743. | 1-(1,3-benzodioxol-5-yl)-N-(1,3-benzodioxol-5-ylmethyl)-N-[(3-butyl-2-phenylpyridin- |
| | 4-yl)methyl]methanamine |
| 1744. | 1-(1,3-benzodioxol-5-yl)-N-benzyl-N-[(3-butyl-2-phenylpyridin-4- |
| | yl)methyl]methanamine |
| 1745. | N-(1,3-benzodioxol-5-ylmethyl)-N-benzyl-1,1′-biphenyl-2-carboxamide |
| 1746. | N-(1,3-benzodioxol-5-ylmethyl)-N-(cyclopentylmethyl)-1,1′-biphenyl-2-carboxamide |
| 1747. | N,N-bis(1,3-benzodioxol-5-ylmethyl)-1,1′-biphenyl-2-carboxamide |
| 1748. | N-benzyl-N-[4-(dimethylamino)benzyl]-1,1′-biphenyl-2-carboxamide |
| 1749. | N-(1,3-benzodioxol-5-ylmethyl)-N-benzyl-4′-(trifluoromethyl)-1,1′-biphenyl-2- |
| | carboxamide |
| 1750. | N-benzyl-N-(1-naphthylmethyl)-4′-(trifluoromethyl)-1,1′-biphenyl-2-carboxamide |
| 1751. | N-(1,3-benzodioxol-5-ylmethyl)-N-(cyclopentylmethyl)-4′-(trifluoromethyl)-1,1′- |
| | biphenyl-2-carboxamide |
| 1752. | N-benzyl-N-[4-(dimethylamino)benzyl]-4′-(trifluoromethyl)-1,1′-biphenyl-2- |
| | carboxamide |
| 1753. | N-benzyl-N-(2,3-dihydro-1H-indan-2-yl)-4′-(trifluoromethyl)-1,1′-biphenyl-2- |
| | carboxamide |
| 1754. | N-benzyl-2-methoxy-N-(1-naphthylmethyl)-1,1′-biphenyl-3-carboxamide |
| 1755. | N-(1,3-benzodioxol-5-ylmethyl)-N-(cyclopentylmethyl)-2-methoxy-1,1′-biphenyl-3- |
| | carboxamide |
| 1756. | N-benzyl-N-(2,3-dihydro-1H-indan-2-yl)-2-methoxy-1,1′-biphenyl-3-carboxamide |
| 1757. | 4-({(1,3-benzodioxol-5-ylmethyl)[(3-butyl-2-phenylpyridin-4- |
| | yl)methyl]amino}methyl)-3-chlorophenol |
| 1758. | N-(1,3-benzodioxol-4-ylmethyl)-N-benzyl-1-butyl-2-phenyl-4,5,6,7-tetrahydro-1H- |
| | benzimidazol-7-amine |
| 1759. | 4-{[benzyl(1-butyl-2-phenyl-4,5,6,7-tetrahydro-1H-benzimidazol-7-yl)amino]methyl}- |
| | 3-chlorophenol |
| 1760. | N-(1,3-benzodioxol-50-ylmethyl)-N-benzyl-3-butyl-2-phenyl-3,4,5,6- |
| | tetrahydrocyclopenta[d]imidazol-4-amine |
| 1761. | N-(1,3-benzodioxol-5-ylmethyl)-N,3-dibutyl-2-phenyl-3,4,5,6- |
| | tetrahydrocyclopenta[d]imidazol-4-amine |
| 1762. | 4-{[benzyl(3-butyl-2-phenyl-3,4,5,6-tetrahydrocyclopenta[d]imidazol-4- |
| | yl)amino]methyl}-3-chlorophenol |
| 1763. | 4-{[butyl(3-butyl-2-phenyl-3,4,5,6-tetrahydrocyclopenta[d]imidazol-4- |
| | yl)amino]methyl}-3-chlorophenol |
| 1764. | 4-({butyl[(3-butyl-2-phenylpyridin-4-yl)methyl]amino}methyl)-3-chlorophenol |
| 1765. | 4-({benzyl[(3-butyl-2-phenylpyridin-4-yl)methyl]amino}methyl)-3-chlorophenol |
| 1766. | 4-{[benzyl(3-butyl-2-phenyl-3,4,5,6-tetrahydrocyclopenta[d]imidazol-4- |
| | yl)amino]methyl}phenol |
| 1767. | 4-{[butyl(3-butyl-2-phenyl-3,4,5,6-tetrahydrocyclopenta[d]imidazol-4- |
| | yl)amino]methyl}phenol |
| 1768. | N-(1,3-benzodioxl-5-ylmethyl)-N-benzyl-1-butyl-5,5-dimethyl-2-phenyl-4,5,6,7- |
| | tetrahydro-1H-benzimidazol-7-amine |
| 1769. | 4-{[butyl(1-butyl-5,5-dimethyl-2-phenyl-4,5,6,7-tetrahydro-1H-benzimidazol-7- |
| | yl)amino]methyl}-3-chlorophenol |
| 1770. | 3-{[butyl(1-butyl-5,5-dimethyl-2-phenyl-4,5,6,7-tetrahydro-1H-benzimidazol-7- |
| | yl)amino]methyl}phenol |
| 1771. | 4-{[butyl(1-butyl-5,5-dimethyl-2-phenyl-4,5,6,7-tetrahydro-1H-benzimidazol-7- |
| | yl)amino]methyl}phenol |
| 1772. | 4-{[benzyl(1-butyl-5,5-dimethyl-2-phenyl-4,5,6,7-tetrahydro-1H-benzimidazol-7- |
| | yl)amino]methyl}-3-chlorophenol |
| 1773. | N-(1,3-benzodioxol-5-ylmethyl)-N-benzyl-2-(1H-imidazol-1-yl)benzamide |
| 1774. | methyl 4-{[butyl(1-butyl-5,5-dimethyl-2-phenyl-4,5,6,7-tetrahydro-1H-benzimidazol-7- |
| | yl)amino]methyl}benzoate |
| 1775. | 4-{[butyl(1-butyl-5,5-dimethyl-2-phenyl-4,5,6,7-tetrahydro-1H-benzimidazol-7- |
| | yl)amino]methyl}benzoic acid |
| 1776. | N-(2,3-dihydro-1H-indan-2-yl)-N-(2-fluorobenzyl)-2-[1-(2-methylphenyl)-3,4- |
| | dihydroisoquinolin-2(1H)-yl]acetamide |
| 1777. | N-(2,3-dihydro-1H-indan-2-yl)-N-(2-fluorobenzyl)-2-[1-(2-fluorophenyl)-3,4- |
| | dihydroisoquinolin-2(1H)-yl]acetamide |
| 1778. | 2-[1-(2-chlorophenyl)-3,4-dihydroisoquinolin-2(1H)-yl]-N-(2,3-dihydro-1H-indan-2- |
| | yl)-N-(2-fluorobenzyl)acetamide |
| 1779. | N-(2,3-dihydro-1H-indan-2-yl)-N-(2-fluorobenzyl)-2-[1-[2-(trifluoromethyl)phenyl]- |
| | 3,4-dihydroisoquinolin-2(1H)-yl]acetamide |
| 1780. | N-(2,3-dihydro-1H-indan-2-yl)-N-(2-fluorobenzyl)-2-(1-phenyl-3,4-dihydroisoquinolin- |
| | 2(1H)-yl)acetamide |
| 1781. | 2-[1-(4-chlorophenyl)-3,4-dihydroisoquinolin-2(1H)-yl]-N-(2,3-dihydro-1H-indan-2- |
| | yl)-N-(2-fluorobenzyl)acetamide |
| 1782. | N,N-dibenzyl-4′-fluoro-1,1′-biphenyl-2-carboxamide |
| 1783. | 3′-(acetylamino)-N,N-dibenzyl-1,1′-biphenyl-2-carboxamide |
| 1784. | N,N-dibenzyl-4′-(trifluoromethoxy)-1,1′-biphenyl-2-carboxamide |
| 1785. | N,N-dibenzyl-2′-(methylthio)-1,1′-biphenyl-2-carboxamide |
| 1786. | N,N-dibenzyl-4′-(ethylthio)-1,1′-biphenyl-2-carboxamide |
| 1787. | N,N-dibenzyl-3′-fluoro-1,1′-biphenyl-2-carboxamide |
| 1788. | N,N-dibenzyl-3′,4′-dimethyl-1,1′-biphenyl-2-carboxamide |
| 1789. | N,N-dibenzyl-2-thien-3-ylbenzamide |
| 1790. | N,N-dibenzyl-4′-(trifluoromethyl)-1,1′-biphenyl-2-carboxamide |
| 1791. | N,N-dibenzyl-2′-methoxy-1,1′-biphenyl-2-carboxamide |
| 1792. | N,N-dibenzyl-4′-methoxy-1,1′-biphenyl-2-carboxamide |
| 1793. | N,N-dibenzyl-2′,4′-dimethoxy-1,1′-biphenyl-2-carboxamide |
| 1794. | N,N-dibenzyl-3′,4′-dimethoxy-1,1′-biphenyl-2-carboxamide |
| 1795. | N,N-dibenzyl-1,1′-biphenyl-2-carboxamide |
| 1796. | N,N-dibenzyl-2′-chloro-1,1′-biphenyl-2-carboxamide |
| 1797. | N,N-dibenzyl-3′-chloro-1,1′-biphenyl-2-carboxamide |
| 1798. | N,N-dibenzyl-4′-chloro-1,1′-biphenyl-2-carboxamide |
| 1799. | N,N-dibenzyl-2′,3′-dichloro-1,1′-biphenyl-2-carboxamide |
| 1800. | N,N-dibenzyl-2′,4′-dichloro-1,1′-biphenyl-2-carboxamide |
| 1801. | N,N-dibenzyl-2′-methyl-1,1′-biphenyl-2-carboxamide |
| 1802. | N,N-dibenzyl-3′-methyl-1,1′-biphenyl-2-carboxamide |
| 1803. | N,N-dibenzyl-4′-methyl-1,1′-biphenyl-2-carboxamide |
| 1804. | N,N-dibenzyl-3′,5′-dichloro-1,1′-biphenyl-2-carboxamide |
| 1805. | N-benzyl-N-[2-(4-methoxyphenyl)-1-methylethyl]-4′-methyl-1,1′-biphenyl-2- |
| | carboxamide |
| 1806. | N-benzyl-N-(2,3-dihydro-1H-indan-2-yl)-4′-methyl-1,1′-biphenyl-2-carboxamide |
| 1807. | N-benzyl-N-(2,3-dihydro-1H-indan-2-yl)-2-(4-methylthien-2-yl)benzamide |
| 1808. | N-benzyl-N-(2,3-dihydro-1H-indan-2-yl)-1,1′-biphenyl-2-carboxamide |
| 1809. | N-benzyl-N-(2,3-dihydro-1H-indan-1-yl)-2′-fluoro-1,1′-biphenyl-2-carboxamide |
| 1810. | N-benzyl-N-(2,3-dihydro-1H-indan-2-yl)-3′-methoxy-1,1′-biphenyl-2-carboxamide |
| 1811. | N-benzyl-N-(2,3-dihydro-1H-indan-2-yl)-2-thien-2-ylbenzamide |
| 1812. | N-(1,3-benzodioxol-5-ylmethyl)-N-(2,3-dihydro-1H-indan-2-yl)-3′-methoxy-1,1′- |
| | biphenyl-2-carboxamide |
| 1813. | N-(1,3-benzodioxol-5-ylmethyl)-N-(2,3-dihydro-1H-indan-2-yl)-9H-fluorene-4- |
| | carboxamide |
| 1814. | N-benzyl-N-(2,3-dihydro-1H-indan-2-yl)-4-methoxy-1,1′-biphenyl-2-carboxamide |
| 1815. | N-benzyl-N-(2,3-dihydro-1H-indan-2-yl)-3-methoxy-1,1′-biphenyl-2-carboxamide |
| 1816. | N-benzyl-N-(2,3-dihydro-1H-indan-2-yl)-4-hydroxy-1,1′-biphenyl-2-carboxamide |
| 1817. | N-benzyl-N-(2,3-dihydro-1H-indan-2-yl)-3-hydroxy-1,1′-biphenyl-2-carboxamide |
| 1818. | N-benzyl-N-(2,3-dihydro-1H-indan-2-yl)-5-methoxy-2-(5-methylthien-2-yl)benzamide |
| 1819. | N-benzyl-N-(5-methoxy-2,3-dihydro-1H-indan-2-yl)-1,1′-biphenyl-2-carboxamide |
| 1820. | N-benzyl-N-(5-methyl-2,3-dihydro-1H-indan-2-yl)-1,1′-biphenyl-2-carboxamide |
| 1821. | N-(2,3-dihydro-1H-indan-2-yl)-N-(2-methylbenzyl)-1,1′-biphenyl-2-carboxamide |
| 1822. | N-(2,3-dihydro-1H-indan-2-yl)-N-(3-methylbenzyl)-1,1′-biphenyl-2-carboxamide |
| 1823. | N-(2,3-dihydro-1H-indan-2-yl)-N-(4-methylbenzyl)-1,1′-biphenyl-2-carboxamide |
| 1824. | N-(2,3-dihydro-1H-indan-2-yl)-N-(4-fluorobenzyl)-1,1′-biphenyl-2-carboxamide |
| 1825. | N-(2,3-dihydro-1H-indan-2-yl)-N-(3-fluorobenzyl)-1,1′-biphenyl-2-carboxamide |
| 1826. | N-(2,3-dihydro-1H-indan-2-yl)-N-(2-fluorobenzyl)-1,1′-biphenyl-2-carboxamide |
| 1827. | N-(1,3-benzodioxol-5-ylmethyl)-N-(2,3-dihydro-1H-indan-2-yl)-1,1′-biphenyl-2- |
| | carboxamide |
| 1828. | N-(3,4-difluorobenzyl)-N-(2,3-dihydro-1H-indan-2-yl)-1,1′-biphenyl-2-carboxamide |
| 1829. | N-(2,3-dihydro-1H-indan-2-yl)-N-(2-methoxybenzyl)-2-thien-3-ylbenzamide |
| 1830. | N-(2,3-dihydro-1H-indan-2-yl)-2′-fluoro-N-(2-methoxybenzyl)-1,1′-biphenyl-2- |
| | carboxamide |
| 1831. | N-(2,3-dihydro-1H-indan-2-yl)-N-(4-methoxybenzyl)-2-thien-3-ylbenzamide |
| 1832. | N-(2,3-dihydro-1H-indan-2-yl)-N-(3,5-dimethoxybenzyl)-2-thien-3-ylbenzamide |
| 1833. | N-(2,3-dihydro-1H-indan-2-yl)-2-thien-3-yl-N-(3-thien-3-ylbenzyl)benzamide |
| 1834. | N-(2,3-dihydro-1H-indan-2-yl)-N-(2-naphthylmethyl)-2-thien-3-ylbenzamide |
| 1835. | N,N-dibenzyl-2′-fluoro-1,1′-biphenyl-2-carboxamide |
| 1836. | N-(2,3-dihydro-1H-indan-2-yl)-4′-methyl-N-(4-methylbenzyl)-1,1′-biphenyl-2- |
| | carboxamide |
| 1837. | N-(2,3-dihydro-1H-indan-2-yl)-3′-methoxy-N-(4-methylbenzyl)-1,1′-biphenyl-2- |
| | carboxamide |
| 1838. | N-(2,3-dihydro-1H-indan-2-yl)-2′-fluoro-N-(4-methylbenzyl)-1,1′-biphenyl-2- |
| | carboxamide |
| 1839. | N-(2,3-dihydro-1H-indan-2-yl)-N-(4-fluorobenzyl)-4′-methyl-1,1′-biphenyl-2- |
| | carboxamide |
| 1840. | N-(2,3-dihydro-1H-indan-2-yl)-N-(4-flurorobenzyl)-3′-methoxy-1,1′-biphenyl-2- |
| | carboxamide |
| 1841. | N-(2,3-dihydro-1H-indan-2-yl)-2′-fluoro-N-(4-fluorobenzyl)-1,1′-biphenyl-2- |
| | carboxamide |
| 1842. | N-(2,3-dihydro-1H-indan-2-yl)-N-(2-flurobenzyl)-4′-methyl-1,1′-biphenyl-2- |
| | carboxamide |
| 1843. | N-(2,3-dihydro-1H-indan-2-yl)-N-(3-fluorobenzyl)-4′-methyl-1,1′-biphenyl-2- |
| | carboxamide |
| 1844. | N-(2,3-dihydro-1H-indan-2-yl)-2′-fluoro-N-(3-fluorobenzyl)-1,1′-biphenyl-2- |
| | carboxamide |
| 1845. | N-(2,3-dihydro-1H-indan-2-yl)-4′-methyl-N-(3-methylbenzyl)-1,1′-biphenyl-2- |
| | carboxamide |
| 1846. | N-(2,3-dihydro-1H-indan-2-yl)-3′-methoxy-N-(3-methylbenzyl)-1,1′-biphenyl-2- |
| | carboxamide |
| 1847. | N-(2,3-dihydro-1H-indan-2-yl)-2′-fluoro-N-(3-methylbenzyl)-1,1′-biphenyl-2- |
| | carboxamide |
| 1848. | N-(2,3-dihydro-1H-indan-2-yl)-4′-methyl-N-(2-methylbenzyl)-1,1′-biphenyl-2- |
| | carboxamide |
| 1849. | N-(2,3-dihydro-1H-indan-2-yl)-3-methoxy-N-(3-methoxybenzyl)-1,1′-biphenyl-2- |
| | carboxamide |
| 1850. | N-(2,3-dihydro-1H-indan-2-yl)-5-methoxy-N-(3-methoxybenzyl)-2-(5-methylthien-2- |
| | yl)benzamide |
| 1851. | N-(2,3-dihydro-1H-indan-2-yl)-4-methoxy-N-(3-methoxybenzyl)-1,1′-biphenyl-2- |
| | carboxamide |
| 1852. | N-(2,3-dihydro-1H-indan-2-yl)-5-methoxy-N-(3-methoxybenzyl)-2-thien-3- |
| | ylbenzamide |
| 1853. | N-(2,3-dihydro-1H-indan-2-yl)-4-methoxy-N-(2-methylbenzyl)-1,1′-biphenyl-2- |
| | carboxamide |
| 1854. | N-(2,3-dihydro-1H-indan-2-yl)-4-methoxy-N-(3-methylbenzyl)-1,1′-biphenyl-2- |
| | carboxamide |
| 1855. | N-(2,3-dihydro-1H-indan-2-yl)-4-methoxy-N-(4-methylbenzyl)-1,1′-biphenyl-2- |
| | carboxamide |
| 1856. | N-(2,3-dihydro-1H-indan-2-yl)-N-(2-fluorobenzyl)-4-methoxy-1,1′-biphenyl-2- |
| | carboxamide |
| 1857. | N-(2,3-dihydro-1H-indan-2-yl)-N-(3-fluorobenzyl)-4-methoxy-1,1′-biphenyl-2- |
| | carboxamide |
| 1858. | N-(2,3-dihydro-1H-indan-2-yl)-N-(4-fluorobenzyl)-4-methoxy-1,1′-biphenyl-2- |
| | carboxamide |
| 1859. | N-(3,4-difluorobenzyl)-N-(2,3-dihydro-1H-indan-2-yl)-4-methoxy-1,1′-biphenyl-2- |
| | carboxamide |
| 1860. | N-benzyl-N-(2,3-dihydro-1H-indan-2-yl)-3-fluoro-1,1′-biphenyl-2-carboxamide |
| 1861. | N-benzyl-N-(2,3-dihydro-1H-indan-2-yl)-3-methyl-1,1′-biphenyl-2-carboxamide |
| 1862. | N-benzyl-N-(2,3-dihydro-1H-indan-2-yl)-2-fluoro-6-(5-methylthien-2-yl)benzamide |
| 1863. | N-benzyl-N-(2,3-dihydro-1H-indan-2-yl)-2-methyl-6-(5-methylthien-2-yl)benzamide |
| 1864. | N-benzyl-N-(2,3-dihydro-1H-indan-2-yl)-4-methyl-1,1′-biphenyl-2-carboxamide |
| 1865. | N-benzyl-4-chloro-N-(2,3-dihydro-1H-indan-2-yl)-1,1′-biphenyl-2-carboxamide |
| 1866. | N-benzyl-N-(2,3-dihydro-1H-indan-2-yl)-5-methyl-2-thien-3-ylbenzamide |
| 1867. | N-benzyl-5-chloro-N-(2,3-dihydro-1H-indan-2-yl)-2-thien-3-ylbenzamide |
| 1868. | N-(2,3-dihydro-1H-indan-2-yl)-3-methyl-N-(2-methylbenzyl)-1,1′-biphenyl-2- |
| | carboxamide |
| 1869. | N-(2,3-dihydro-1H-indan-2-yl)-3-methyl-N-(3-methylbenzyl)-1,1′-biphenyl-2- |
| | carboxamide |
| 1870. | N-(2,3-dihydro-1H-indan-2-yl)-3-methyl-N-(4-methylbenzyl)-1,1′-biphenyl-2- |
| | carboxamide |
| 1871. | N-(2,3-dihydro-1H-indan-2-yl)-N-(2-fluorobenzyl)-3-methyl-1,1′-biphenyl-2- |
| | carboxamide |
| 1872. | N-(2,3-dihydro-1H-indan-2-yl)-N-(3-fluorobenzyl)-3-methyl-1,1′-biphenyl-2- |
| | carboxamide |
| 1873. | N-(2,3-dihydro-1H-indan-2-yl)-N-(4-fluorobenzyl)-3-methyl-1,1′-biphenyl-2- |
| | carboxamide |
| 1874. | N-(3,4-difluorobenzyl)-N-(2,3-dihydro-1H-indan-2-yl)-3-methyl-1,1′-biphenyl-2- |
| | carboxamide |
| 1875. | N-(2,3-dihydro-1H-indan-2-yl)-N-(3-methoxybenzyl)-3-methyl-1,1′-biphenyl-2- |
| | carboxamide |
| 1876. | N-(2,3-dihydro-1H-indan-2-yl)-N-(4-methoxybenzyl)-3-methyl-1,1′-biphenyl-2- |
| | carboxamide |
| 1877. | N-(1,3-benzodioxol-5-ylmethyl)-N-(2,3-dihydro-1H-indan-2-yl)-3-methyl-1,1′- |
| | biphenyl-2-carboxamide |
| 1878. | N-(2,3-dihydro-1H-indan-2-yl)-3-methyl-N-(2-phenylethyl)-1,1′-biphenyl-2- |
| | carboxamide |
| 1879. | 4-chloro-N-(2,3-dihydro-1H-indan-2-yl)-N-(2-methylbenzyl)-1,1′-biphenyl-2- |
| | carboxamide |
| 1880. | 4-chloro-N-(2,3-dihydro-1H-indan-2-yl)-N-(3-methylbenzyl)-1,1′-biphenyl-2- |
| | carboxamide |
| 1881. | 4-chloro-N-(2,3-dihydro-1H-indan-2-yl)-N-(4-methylbenzyl)-1,1′-biphenyl-2- |
| | carboxamide |
| 1882. | 4-chloro-N-(2,3-dihydro-1H-indan-2-yl)-N-(2-fluorobenzyl)-1,1′-biphenyl-2- |
| | carboxamide |
| 1883. | 4-chloro-N-(2,3-dihydro-1H-indan-2-yl)-N-(3-fluorobenzyl)-1,1′-biphenyl-2- |
| | carboxamide |
| 1884. | 4-chloro-N-(2,3-dihydro-1H-indan-2-yl)-N-(4-fluorobenzyl)-1,1′-biphenyl-2- |
| | carboxamide |
| 1885. | 4-chloro-N-(3,4-difluorobenzyl)-N-(2,3-dihydro-1H-indan-2-yl)-1,1′-biphenyl-2- |
| | carboxamide |
| 1886. | 4-chloro-N-(2,3-dihydro-1H-indan-2-yl)-N-(4-methoxybenzyl)-1,1′-biphenyl-2- |
| | carboxamide |
| 1887. | N-(1,3-benzodioxol-5-ylmethyl)-4-chloro-N-(2,3-dihydro-1H-indan-2-yl)-1,1′- |
| | biphenyl-2-carboxamide |
| 1888. | 4-chloro-N-(2,3-dihydro-1H-indan-2-yl)-N-(3-methoxybenzyl)-1,1′-biphenyl-2- |
| | carboxamide |
| 1889. | 4-chloro-N-(2,3-dihydro-1H-indan-2-yl)-N-(2-phenylethyl)-1,1′-biphenyl-2- |
| | carboxamide |
| 1890. | N-benzyl-4-chloro-N-(4-hydroxy-3,5-dimethylbenzyl)-1,1′-biphenyl-2-carboxamide |
| 1891. | N-benzyl-N-{(1S,2S)-2-[4-(2-methoxyphenyl)piperazin-1-yl]cyclohexyl}-1,1′- |
| | biphenyl-2-carboxamide |
| 1892. | N-benzyl-4-chloro-N-(2,3-dihydro-1-benzofuran-6-ylmethyl)-1,1′-biphenyl-2- |
| | carboxamide |
| 1893. | 4-chloro-N-(3-methoxybenzyl)-N-(2-phenylethyl)-1,1′-biphenyl-2-carboxamide |
| 1894. | N-benzyl-4-chloro-N-(1-naphthylmethyl)-1,1′-biphenyl-2-carboxamide |
| 1895. | N-benzyl-N-(4-hydroxy-3,5-dimethylbenzyl)-2-(5-methylthien-2-yl)benzamide |
| 1896. | N-benzyl-N-[4-(difluoromethoxy)benzyl]-2-(5-methylthien-2-yl)benzamide |
| 1897. | N-benzyl-N-(2-chloro-4-hydroxybenzyl)-2-(5-methylthien-2-yl)benzamide |
| 1898. | N-benzyl-N-(4-hydroxy-3,5-dimethylbenzyl)-1,1′-biphenyl-2-carboxamide |
| 1899. | N-benzyl-N-[4-(difluoromethoxy)benzyl]-1,1′-biphenyl-2-carboxamide |
| 1900. | N-benzyl-2-(5-methylthien-2-yl)-N-[3-(trifluoromethyl)benzyl]benzamide |
| 1901. | 1-(1,3-benzodioxol-5-yl)-N-(1,3-benzodioxol-5-ylmethyl)-N-{[1-butyl-4-(methylthio)- |
| | 2-phenyl-1H-imidazol-5-yl]methyl}methanamine |
| 1902. | 1-(1,3-benzodioxol-5-yl)-N-(1,3-benzodioxol-5-ylmethyl)-N-{[1-butyl-4- |
| | (methylsulfonyl)-2-phenyl-1H-imidazol-5-yl]methyl}methanamine |
| 1903. | 1-(1,3-benzodioxol-5-yl)-N-{[1-butyl-4-(methylsulfonyl)-2-phenyl-1H-imidazol-5- |
| | yl]methyl}-N-[4-(difluoromethoxy)benzyl]methanamine |
| 1904. | 1-(1,3-benzodioxol-5-yl)-N-{[1-butyl-4-(4-methoxyphenyl)-2-phenyl-1H-imidazol-5- |
| | yl]methyl}-N-[3-(1,1,2,2-tetrafluoroethoxy)benzyl]methanamine |
| 1905. | 1-(1,3-benzodioxol-5-yl)-N-{[1-butyl-4-(4-methoxyphenyl)-2-phenyl-1H-imidazol-5- |
| | yl]methyl}-N-(3-ethoxybenzyl)methanamine |
| 1906. | 1-(1,3-benzodioxol-5-yl)-N-{[1-butyl-4-(4-ethoxyphenyl)-2-phenyl-1H-imidazol-5- |
| | yl]methyl}-N-[3-(1,1,2,2-tetrafluoroethoxy)benzyl]methanamine |
| 1907. | 1-(1,3-benzodioxol-5-yl)-N-({1-butyl-4-[4-(methylthio)phenyl]-2-phenyl-1H-imidazol- |
| | 5-yl}methyl)-N-[3-(1,1,2,2-tetrafluoroethoxy)benzyl]methanamine |
| 1908. | 1-(1,3-benzodioxol-5-yl)-N-({1-butyl-4-[4-(ethylthio)phenyl]-2-phenyl-1H-imidazol-5- |
| | yl}methyl)-N-[3-(1,1,2,2-tetrafluoroethoxy)benzyl]methanamine |
| 1909. | 1-(1,3-benzodioxol-5-yl)-N-{[1-butyl-4-(3-methoxyphenyl)-2-phenyl-1H-imidazol-5- |
| | yl]methyl}-N-[3-(1,1,2,2-tetrafluoroethoxy)benzyl]methanamine |
| 1910. | 1-(1,3-benzodioxol-5-yl)-N-{[1-butyl-4-(3-ethoxyphenyl)-2-phenyl-1H-imidazol-5- |
| | yl]methyl}-N-[3-(1,1,2,2-tetrafluoroethoxy)benzyl]methanamine |
| 1911. | 1-(1,3-benzodioxol-5-yl)-N-({1-butyl-4-[3-(methylthio)phenyl]-2-phenyl-1H-imidazol- |
| | 5-yl}methyl)-N-[3-(1,1,2,2-tetrafluoroethoxy)benzyl]methanamine |
| 1912. | 1-(1,3-benzodioxol-5-yl)-N-{[1-butyl-4-(3-methylphenyl)-2-phenyl-1H-imidazol-5- |
| | yl]methyl}-N-[3-(1,1,2,2-tetrafluoroethoxy)benzyl]methanamine |
| 1913. | 1-(1,3-benzodioxol-5-yl)-N-{[1-butyl-4-(4-methylphenyl)-2-phenyl-1H-imidazol-5- |
| | yl]methyl}-N-[3-(1,1,2,2-tetrafluoroethoxy)benzyl]methanamine |
| 1914. | 1-(1,3-benzodioxol-5-yl)-N-{[1-butyl-4-(3,4-dimethylphenyl)-2-phenyl-1H-imidazol-5- |
| | yl]methyl}-N-[3-(1,1,2,2-tetrafluoroethoxy)benzyl]methanamine |
| 1915. | 1-(1,3-benzodioxol-5-yl)-N-{[1-butyl-4-(3,5-dimethylphenyl)-2-phenyl-1H-imidazol-5- |
| | yl]methyl}-N-[3-(1,1,2,2-tetrafluoroethoxy)benzyl]methanamine |
| 1916. | 1-(1,3-benzodioxol-5-yl)-N-{[1-butyl-4-(3-fluorophenyl)-2-phenyl-1H-imidazol-5- |
| | yl]methyl}-N-[3-(1,1,2,2-tetrafluoroethoxy)benzyl]methanamine |
| 1917. | 1-(1,3-benzodioxol-5-yl)-N-{[1-butyl-4-(4-fluorophenyl)-2-phenyl-1H-imidazol-5- |
| | yl]methyl}-N-[3-(1,1,2,2-tetrafluoroethoxy)benzyl]methanamine |
| 1918. | 1-(1,3-benzodioxol-5-yl)-N-{[1-butyl-4-(3,4-difluorophenyl)-2-phenyl-1H-imidazol-5- |
| | yl]methyl}-N-[3-(1,1,2,2-tetrafluoroethoxy)benzyl]methanamine |
| 1919. | 1-(1,3-benzodioxol-5-yl)-N-{[1-butyl-4-(3,5-difluorophenyl)-2-phenyl-1H-imidazol-5- |
| | yl]methyl}-N-[3-(1,1,2,2-tetrafluoroethoxy)benzyl]methanamine |
| 1920. | 1-(1,3-benzodioxol-5-yl)-N-{[1-butyl-4-(4-ethoxyphenyl)-2-phenyl-1H-imidazol-5- |
| | yl]methyl}-N-(3-ethoxybenzyl)methanamine |
| 1921. | 1-(1,3-benzodioxol-5-yl)-N-({1-butyl-4-[4-(methylthio)phenyl]-2-phenyl-1H-imidazol- |
| | 5-yl}methyl)-N-(3-ethoxybenzyl)methanamine |
| 1922. | 1-(1,3-benzodioxol-5-yl)-N-({1-butyl-4-[4-(ethylthio)phenyl]-2-phenyl-1H-imidazol-5- |
| | yl}methyl)-N-(3-ethoxybenzyl)methanamine |
| 1923. | 1-(1,3-benzodioxol-5-yl)-N-{[1-butyl-4-(3-methoxyphenyl)-2-phenyl-1H-imidazol-5- |
| | yl]methyl}-N-(3-ethoxybenzyl)methanamine |
| 1924. | 1-(1,3-benzodioxol-5-yl)-N-{[1-butyl-4-(3-ethoxyphenyl)-2-phenyl-1H-imidazol-5- |
| | yl]methyl}-N-(3-ethoxybenzyl)methanamine |
| 1925. | 1-(1,3-benzodioxol-5-yl)-N-({1-butyl-4-[3-(methylthio)phenyl]-2-phenyl-1H-imidazol- |
| | 5-yl}methyl)-N-(3-ethoxybenzyl)methanamine |
| 1926. | 1-(1,3-benzodioxol-5-yl)-N-{[1-butyl-4-(3-methylphenyl)-2-phenyl-1H-imidazol-5- |
| | yl]methyl}-N-(3-ethoxybenzyl)methanamine |
| 1927. | 1-(1,3-benzodioxol-5-yl)-N-{[1-butyl-4-(4-methylphenyl)-2-phenyl-1H-imidazol-5- |
| | yl]methyl}-N-(3-ethoxybenzyl)methanamine |
| 1928. | 1-(1,3-benzodioxol-5-yl)-N-{[1-butyl-4-(3,4-dimethylphenyl)-2-phenyl-1H-imidazol-5- |
| | yl]methyl}-N-(3-ethoxybenzyl)methanamine |
| 1929. | 1-(1,3-benzodioxol-5-yl)-N-{[1-butyl-4-(3,5-dimethylphenyl)-2-phenyl-1H-imidazol-5- |
| | yl]methyl}-N-(3-ethoxybenzyl)methanamine |
| 1930. | 1-(1,3-benzodioxol-5-yl)-N-{[1-butyl-4-(3-fluorophenyl)-2-phenyl-1H-imidazol-5- |
| | yl]methyl}-N-(3-ethoxybenzyl)methanamine |
| 1931. | 1-(1,3-benzodioxol-5-yl)-N-{[1-butyl-4-(4-fluorophenyl)-2-phenyl-1H-imidazol-5- |
| | yl]methyl}-N-(3-ethoxybenzyl)methanamine |
| 1932. | 1-(1,3-benzodioxol-5-yl)-N-{[1-butyl-4-(3,4-difluorophenyl)-2-phenyl-1H-imidazol-5- |
| | yl]methyl}-N-(3-ethoxybenzyl)methanamine |
| 1933. | 1-(1,3-benzodioxol-5-yl)-N-{[1-butyl-4-(3,5-difluorophenyl)-2-phenyl-1H-imidazol-5- |
| | yl]methyl}-N-(3-ethoxybenzyl)methanamine |
| 1934. | 1-(1,3-benzodioxol-5-yl)-N-({1-butyl-2-phenyl-4-[3-(trifluoromethyl)phenyl]-1H- |
| | imidazol-5-yl}methyl)-N-(3-ethoxybenzyl)methanamine |
| 1935. | 1-(1,3-benzodioxol-5-yl)-N-({1-butyl-2-phenyl-4-[4-(trifluoromethyl)phenyl]-1H- |
| | imidazol-5-yl}methyl)-N-(3-ethoxybenzyl)methanamine |
| 1936. | 1-(1,3-benzodioxol-5-yl)-N-({1-butyl-2-phenyl-4-[3-(trifluoromethoxy)phenyl]-1H- |
| | imidazol-5-yl}methyl)-N-(3-ethoxybenzyl)methanamine |
| 1937. | 1-(1,3-benzodioxol-5-yl)-N-({1-butyl-2-phenyl-4-[4-(trifluoromethoxy)phenyl]-1H- |
| | imidazol-5-yl}methyl)-N-(3-ethoxybenzyl)methanamine |
| 1938. | 1-(1,3-benzodioxol-5-yl)-N-({1-butyl-4-[(1E)-pent-1-enyl]-2-phenyl-1H-imidazol-5- |
| | yl}methyl)-N-(3-ethoxybenzyl)methanamine |
| 1939. | 1-(1,3-benzodioxol-5-yl)-N-({1-butyl-2-phenyl-4-[3-(trifluoromethyl)phenyl]-1H- |
| | imidazol-5-yl}methyl)-N-[3-(1,1,2,2-tetrafluoroethoxy)benzyl]methanamine |
| 1940. | 1-(1,3-benzodioxol-5-yl)-N-({1-butyl-2-phenyl-4-[4-(trifluoromethyl)phenyl]-1H- |
| | imidazol-5-yl}methyl)-N-[3-(1,1,2,2-tetrafluoroethoxy)benzyl]methanamine |
| 1941. | 1-(1,3-benzodioxol-5-yl)-N-({1-butyl-2-phenyl-4-[3-(trifluoromethoxy)phenyl]-1H- |
| | imidazol-5-yl}methyl)-N-[3-(1,1,2,2-tetrafluoroethoxy)benzyl]methanamine |
| 1942. | 1-(1,3-benzodioxol-5-yl)-N-({1-butyl-2-phenyl-4-[4-(trifluoromethoxy)phenyl]-1H- |
| | imidazol-5-yl}methyl)-N-[3-(1,1,2,2-tetrafluoroethoxy)benzyl]methanamine |
| 1943. | 1-(1,3-benzodioxol-5-yl)-N-({1-butyl-4-[(1E)-pent-1-enyl]-2-phenyl-1H-imidazol-5- |
| | yl}methyl)-N-[3-(1,1,2,2-tetrafluoroethoxy)benzyl]methanamine |
| 1944. | 1-(1,3-benzodioxol-5-yl)-N-{[1-butyl-4-(4-chlorophenyl)-2-phenyl-1H-imidazol-5- |
| | yl]methyl}-N-(3-ethoxybenzyl)methanamine |
| 1945. | 1-(1,3-benzodioxol-5-yl)-N-{[1-butyl-4-(3-chlorophenyl)-2-phenyl-1H-imidazol-5- |
| | yl]methyl}-N-(3-ethoxybenzyl)methanamine |
| 1946. | 1-(1,3-benzodioxol-5-yl)-N-{[1-butyl-4-(3-isopropylphenyl)-2-phenyl-1H-imidazol-5- |
| | yl]methyl}-N-(3-ethoxybenzyl)methanamine |
| 1947. | 1-(1,3-benzodioxol-5-yl)-N-{[1-butyl-4-(2,4-difluorophenyl)-2-phenyl-1H-imidazol-5- |
| | yl]methyl}-N-(3-ethoxybenzyl)methanamine |
| 1948. | 1-(1,3-benzodioxol-5-yl)-N-{[1-butyl-4-(4-chlorophenyl)-2-phenyl-1H-imidazol-5- |
| | yl]methyl}-N-[3-(1,1,2,2-tetrafluoroethoxy)benzyl]methanamine |
| 1949. | 1-(1,3-benzodioxol-5-yl)-N-{[1-butyl-4-(3-chlorophenyl)-2-phenyl-1H-imidazol-5- |
| | yl]methyl}-N-[3-(1,1,2,2-tetrafluoroethoxy)benzyl]methanamine |
| 1950. | 1-(1,3-benzodioxol-5-yl)-N-{[1-butyl-4-(3-isopropylphenyl)-2-phenyl-1H-imidazol-5- |
| | yl]methyl}-N-[3-(1,1,2,2-tetrafluoroethoxy)benzyl]methanamine |
| 1951. | 1-(1,3-benzodioxol-5-yl)-N-{[1-butyl-4-(2,4-difluorophenyl)-2-phenyl-1H-imidazol-5- |
| | yl]methyl}-N-[3-(1,1,2,2-tetrafluoroethoxy)benzyl]methanamine |
| 1952. | 1-(1,3-benzodioxol-5-yl)-N-(1,3-benzodioxol-5-ylmethyl)-N-{[1-butyl-4-(3- |
| | isopropylphenyl)-2-phenyl-1H-imidazol-5-yl]methyl}methanamine |
| 1953. | 1-(1,3-benzodioxol-5-yl)-N-(1,3-benzodioxol-5-ylmethyl)-N-{[1-butyl-4-(3,4- |
| | dimethylphenyl)-2-phenyl-1H-imidazol-5-yl]methyl}methanamine |
| 1954. | 1-(1,3-benzodioxol-5-yl)-N-(1,3-benzodioxol-5-ylmethyl)-N-{[1-butyl-4-(2,4- |
| | difluorophenyl)-2-phenyl-1H-imidazol-5-yl]methyl}methanamine |
| 1955. | 1-(1,3-benzodioxol-5-yl)-N-(1,3-benzodioxol-5-ylmethyl)-N-{[1-butyl-4-(3,5- |
| | difluorophenyl)-2-phenyl-1H-imidazol-5-yl]methyl}methanamine |
| 1956. | 1-(1,3-benzodioxol-5-yl)-N-(1,3-benzodioxol-5-ylmethyl)-N-({1-butyl-2-phenyl-4-[(E)- |
| | 2-phenylethenyl]-1H-imidazol-5-yl}methyl)methanamine |
| 1957. | 1-(1,3-benzodioxol-5-yl)-N-({1-butyl-2-phenyl-4-[(E)-2-phenylethenyl]-1H-imidazol- |
| | 5-yl}methyl)-N-(3-ethoxybenzyl)methanamine |
| 1958. | 1-(1,3-benzodioxol-5-yl)-N-({1-butyl-2-phenyl-4-[(E)-2-phenylethenyl]-1H-imidazol- |
| | 5-yl}methyl)-N-[3-(1,1,2,2-tetrafluoroethoxy)benzyl]methanamine |
| 1959. | N-(1,3-benzodioxol-5-ylmethyl)-N-[(1-butyl-4-chloro-2-phenyl-1H-imidazol-5- |
| | yl)methyl]butan-1-amine |
| 1960. | 4-({(1,3-benzodioxol-5-ylmethyl)[(1-butyl-4-chloro-2-phenyl-1H-imidazol-5- |
| | yl)methyl]amino}methyl)benzamide |
| 1961. | 4-({(1,3-benzodioxol-5-ylmethyl)[(1-butyl-4-chloro-2-phenyl-1H-imidazol-5- |
| | yl)methyl]amino}methyl)benzenesulfonamide |
| 1962. | methyl 4-({(1,3-benzodioxol-5-ylmethyl)[(1-butyl-4-chloro-2-phenyl-1H-imidazol-5- |
| | yl)methyl]amino}methyl)benzoate |
| 1963. | 1-(1,3-benzodioxol-5-yl)-N-[(1-butyl-4-chloro-2-phenyl-1H-imidazol-5-yl)methyl]-N- |
| | (3-methoxybenzyl)methanamine |
| 1964. | 6-{[{[1-butyl-2-phenyl-4-(trifluoromethyl)-1H-imidazol-5- |
| | yl]methyl}(cyclohexylmethyl)amino]methyl}-3,4-dihydroquinolin-2(1H)-one |
| 1965. | 6-[(butyl{[1-butyl-2-phenyl-4-(trifluoromethyl)-1H-imidazol-5- |
| | yl]methyl}amino)methyl]-3,4-dihydroquinolin-2(1H)-one |
| 1966. | 6-[((1,3-benzodioxol-5-ylmethyl){[1-butyl-2-phenyl-4-(trifluoromethyl)-1H-imidazol- |
| | 5-yl]methyl}amino)methyl]-3,4-dihydroquinolin-2(1H)-one |
| 1967. | 6-({benzyl[(1-butyl-2,4-diphenyl-1H-imidazol-5-yl)methyl]amino}methyl)-3,4- |
| | dihydroquinolin-2(1H)-one |
| 1968. | 6-({butyl[(1-butyl-2,4-diphenyl-1H-imidazol-5-yl)methyl]amino}methyl)-3,4- |
| | dihydroquinolin-2(1H)-one |
| 1969. | 6-({(1,3-benzodioxol-5-ylmethyl)[(1-butyl-2,4-diphenyl-1H-imidazol-5- |
| | yl)methyl]amino}methyl)-3,4-dihydroquinolin-2(1H)-one |
| 1970. | 6-{[[(1-butyl-2,4-diphenyl-1H-imidazol-5- |
| | yl)methyl](cyclohexylmethyl)amino]methyl}-3,4-dihydroquinolin-2(1H)-one |
| 1971. | 6-({[(1-butyl-2,4-diphenyl-1H-imidazol-5-yl)methyl][4- |
| | (difluoromethoxy)benzyl]amino}methyl)-3,4-dihydroquinolin-2(1H)-one |
| 1972. | 1-(1-butyl-2-phenyl-1H-imidazol-5-yl)-N,N-bis(2,3-dihydro-1,4-benzodioxin-6- |
| | ylmethyl)methanamine |
| 1973. | 1-[1-butyl-2-phenyl-4-(trifluoromethyl)-1H-imidazol-5-yl]-N,N-bis(2,3-dihydro-1,4- |
| | benzodioxin-6-ylmethyl)methanamine |
| 1974. | 1-(1-butyl-2,4-diphenyl-1H-imidazol-5-yl)-N,N-bis(2,3-dihydro-1,4-benzodioxin-6- |
| | ylmethyl)methanamine |
| 1975. | 1-(1,3-benzodioxol-5-yl)-N-[(1-butyl-4-chloro-2-phenyl-1H-imidazol-5-yl)methyl]-N- |
| | (2,3-dihydro-1,4-benzodioxin-6-ylmethyl)methanamine |
| 1976. | 1-(1,3-benzodioxol-5-yl)-N-[(1-butyl-4-chloro-2-phenyl-1H-imidazol-5-yl)methyl]-N- |
| | (2,3-dihydro-1-benzofuran-5-ylmethyl)methanamine |
| 1977. | N-[(1-butyl-4-chloro-2-phenyl-1H-imidazol-5-yl)methyl]-N,N-bis(2,3-dihydro-1,4- |
| | benzodioxin-6-ylmethyl)amine |
| 1978. | 1-(1,3-benzodioxol-5-yl)-N-{[1-butyl-2-phenyl-4-(trifluoromethyl)-1H-imidazol-5- |
| | yl]methyl}-N-(2,3-dihydro-1,4-benzodioxin-6-ylmethyl)methanamine |
| 1979. | 1-(1,3-benzodioxol-5-yl)-N-{[1-butyl-2-phenyl-4-(trifluoromethyl)-1H-imidazol-5- |
| | yl]methyl}-N-(2,3-dihydro-1-benzofuran-5-ylmethyl)methanamine |
| 1980. | 1-(1,3-benzodioxol-5-yl)-N-(1,3-benzodioxol-5-ylmethyl)-N-{[1-butyl-4-phenyl-2-(1H- |
| | pyrazol-1-yl)-1H-imidazol-5-yl]methyl}methanamine |
| 1981. | 4-[(butyl{[1-butyl-4-(5-methylthien-2-yl)-2-phenyl-1H-imidazol-5- |
| | yl]methyl}amino)methyl]benzamide |
| 1982. | N-{[1-butyl-4-(5-methylthien-2-yl)-2-phenyl-1H-imidazol-5-yl]methyl}-N-(3- |
| | ethoxybenzyl)butan-1-amine |
| 1983. | N-{[1-butyl-4-(5-methylthien-2-yl)-2-phenyl-1H-imidazol-5-yl]methyl}-N-[3-(1,1,2,2- |
| | tetrafluoroethoxy)benzyl]butan-1-amine |
| 1984. | N-(1,3-benzodioxol-5-ylmethyl)-N-{[1-butyl-4-(5-methylthien-2-yl)-2-phenyl-1H- |
| | imidazol-5-yl]methyl}butan-1-amine |
| 1985. | 1-(1,3-benzodioxol-5-yl)-N-(1,3-benzodioxol-5-ylmethyl)-N-[(4-butyl-5-phenyl-4H- |
| | 1,2,4-triazol-3-yl)methyl]methanamine |
| 1986. | 1-(1,3-benzodioxol-5-yl)-N-[(4-butyl-5-phenyl-4H-1,2,4-triazol-3-yl)methyl]-N- |
| | (cyclohexylmethyl)methanamine |
| 1987. | N-(1,3-benzodioxol-5-ylmethyl)-N-[(4-butyl-5-phenyl-4H-1,2,4-triazol-3- |
| | yl)methyl]butan-1-amine |
| 1988. | N-(1,3-benzodioxol-5-ylmethyl)-N-[(1-butyl-2,4-diphenyl-1H-imidazol-5-yl)methyl]-2- |
| | morpholin-4-ylethanamine |
| 1989. | 1-(1,3-benzodioxol-5-yl)-N-(1,3-benzoxazol-2-ylmethyl)-N-[(1-butyl-2,4-diphenyl-1H- |
| | imidazol-5-yl)methyl]methanamine |
| 1990. | 1-(1,3-benzodioxol-5-yl)-N-[(1-butyl-2,4-diphenyl-1H-imidazol-5-yl)methyl]-N- |
| | (tetrahydro-2H-pyran-4-ylmethyl)methanamine |
| 1991. | N-(1,3-benzodioxol-5-ylmethyl)-N-[(1-butyl-2,4-diphenyl-1H-imidazol-5-yl)methyl]-2- |
| | piperidin-1-ylethanamine |
| 1992. | N˜1˜-(1,3-benzodioxol-5-ylmethyl)-N˜1˜-[(1-butyl-2,4-diphenyl-1H-imidazol-5- |
| | yl)methyl]-N˜2˜,N˜2˜-diethylethane-1,2-diamine |
| 1993. | N-(1,3-benzodioxol-5-ylmethyl)-N-[(1-butyl-2,4-diphenyl-1H-imidazol-5-yl)methyl]-2- |
| | thiomorpholin-4-ylethanamine |
| 1994. | 1-(1,3-benzodioxol-5-yl)-N-[(1-butyl-2,4-diphenyl-1H-imidazol-5-yl)methyl]-N-[(2S)- |
| | tetrahydrofuran-2-ylmethyl]methanamine |
| 1995. | N-[(1-butyl-2,4-diphenyl-1H-imidazol-5-yl)methyl]-N-methylchroman-4-amine |
| 1996. | 1-(1,3-benzodioxol-5-yl)-N-(1,3-benzodioxol-5-ylmethyl)-N-[(1-butyl-2-pyrimidin-2- |
| | yl-1H-imidazol-5-yl)methyl]methanamine |
| 1997. | N,N-bis(1,3-benzodioxol-5-ylmethyl)-2-(1-butyl-2,4-diphenyl-1H-imidazol-5- |
| | yl)acetamide |
| 1998. | N,N-bis(1,3-benzodioxol-5-ylmethyl)-2-(1-butyl-2,4-diphenyl-1H-imidazol-5- |
| | yl)ethanamine |
| 1999. | 1-(1,3-benzodioxol-5-yl)-N-(1,3-benzodioxol-5-ylmethyl)-N-[(4-butyl-1-methyl-3- |
| | phenyl-1H-pyrazol-5-yl)methyl]methanamine |
| 2000. | N-(1,3-benzodioxol-5-ylmethyl)-N-[(4-butyl-1-methyl-3-phenyl-1H-pyrazol-5- |
| | yl)methyl]butan-1-amine |
| 2001. | 1-(1,3-benzodioxol-5-yl)-N-[(4-butyl-1-methyl-3-phenyl-1H-pyrazol-5-yl)methyl]-N- |
| | (3-ethoxybenzyl)methanamine |
| 2002. | 1-(1,3-benzodioxol-5-yl)-N-benzyl-N[(4-butyl-1-methyl-3-phenyl-1H-pyrazol-5- |
| | yl)methyl]methanamine |
| 2003. | 1-(1,3-benzodioxol-5-yl)-N-(1,3-benzodioxol-5-ylmethyl)-N-[(4-butyl-1-methyl-5- |
| | phenyl-1H-pyrazol-3-yl)methyl]methanamine |
| 2004. | N-(1,3-benzodioxol-5-ylmethyl)-N-[(4-butyl-1-methyl-5-phenyl-1H-pyrazol-3- |
| | yl)methyl]butan-1-amine |
| 2005. | N,N-bis(1,3-benzodioxol-5-ylmethyl)-3-(1-butyl-2,4-diphenyl-1H-imidazol-5- |
| | yl)propanamide |
| 2006. | 1-(1,3-benzodioxol-5-yl)-N-[(1-butyl-2,4-diphenyl-1H-imidazol-5-yl)methyl]-N-(1,4- |
| | dioxaspiro[4.5]dec-8-ylmethyl)methanamine |
| 2007. | 4-({(1,3-benzodioxol-5-ylmethyl)[(1-butyl-2,4-diphenyl-1H-imidazol-5- |
| | yl)methyl]amino}methyl)cyclohexanone |
| 2008. | N,N-bis(1,3-benzodioxol-5-ylmethyl)-3-(1-butyl-2,4-diphenyl-1H-imidazol-5- |
| | yl)propan-1-amine |
| 2009. | 4-({(1,3-benzodioxol-5-ylmethyl)[(1-butyl-2,4-diphenyl-1H-imidazol-5- |
| | yl)methyl]amino}methyl)-1-methylcyclohexanol |
| 2010. | 1-(1,3-benzodioxol-5-yl)-N-[(1-butyl-2,4-diphenyl-1H-imidazol-5-yl)methyl]-N-[(2- |
| | methyl-1,3-benzoxazol-5-yl)methyl]methanamine |
| 2011. | 1-(1,3-benzodioxol-5-yl)-N-[(1-butyl-2,4-diphenyl-1H-imidazol-5-yl)methyl]-N- |
| | methylethanamine |
| 2012. | 4-({(1,3-benzodioxol-5-ylmethyl)[(1-butyl-2,4-diphenyl-1H-imidazol-5- |
| | yl)methyl]amino}methyl)cyclohexanol |
| 2013. | N-(1,3-benzodioxol-5-ylmethyl)-N-[(5-butyl-1,3-diphenyl-1H-pyrazol-4- |
| | yl)methyl]butan-1-amine |
| 2014. | 1-(1,3-benzodioxol-5-yl)-N-[(5-butyl-1,3-diphenyl-1H-pyrazol-4-yl)methyl]-N-(1H- |
| | imidazol-2-ylmethyl)methanamine |
| 2015. | 1-(1,3-benzodioxol-5-yl)-N-(1,3-benzodioxol-5-ylmethyl)-N-[(5-butyl-1-1,3-diphenyl- |
| | 1H-pyrazol-4-yl)methyl]methanamine |
| 2016. | 1-(1,3-benzodioxol-5-yl)-N-[(1-butyl-2,4-diphenyl-1H-imidazol-5-yl)methyl]-N-[(2- |
| | methyl-1,3-benzothiazol-5-yl)methyl]methanamine |
| 2017. | N-[(1-butyl-2,4-diphenyl-1H-imidazol-5-yl)methyl]-N-[(1-ethyl-3,5-dimethyl-1H- |
| | pyrazol-4-yl)methyl]butan-1-amine |
| 2018. | N-[(1-butyl-2,4-diphenyl-1H-imidazol-5-yl)methyl]-N-[(3,5-dimethyl-1H-pyrazol-4- |
| | yl)methyl]butan-1-amine |
| 2019. | 1-(1,3-benzodioxol-5-yl)-N-[(1-butyl-2-phenyl-4-thien-3-yl-1H-imidazol-5-yl)methyl]- |
| | N-[3-(1,1,2,2-tetrafluoroethoxy)benzyl]methanamine |
| 2020. | methyl 2-amino-4-[(butyl{[1-butyl-4-(3-methoxyphenyl)-2-phenyl-1H-imidazol-5- |
| | yl]methyl}amino)methyl]benzoate |
| 2021. | 1-(1,3-benzodioxol-5-yl)-N-[(1-butyl-2,4-diphenyl-1H-imidazol-5-yl)methyl]-N-(2,3- |
| | dihydro-1,4-benzodioxin-6-ylmethyl)methanamine |
| 2022. | 1-(1,3-benzodioxol-5-yl)-N-[(1-butyl-2,4-diphenyl-1H-imidazol-5-yl)methyl]-N-(2,3- |
| | dihydro-1-benzofuran-5-ylmethyl)methanamine |
| 2023. | 1-(1,3-benzodioxol-5-yl)-N-[(1-butyl-2-phenyl-4-thien-3-yl-1H-imidazol-5-yl)methyl]- |
| | N-(3-ethoxybenzyl)methanamine |
| 2024. | 1-(1,3-benzodioxol-5-yl)-N-(1,3-benzodioxol-5-ylmethyl)-N-{[1-butyl-2-(4- |
| | fluorophenyl)-4-phenyl-1H-imidazol-5-yl]methyl}methanamine |
| 2025. | 1-(1-butyl-2-phenyl-1H-imidazol-5-yl)-N-(2,3-dihydro-1,4-benzodioxin-6-ylmethyl)-N- |
| | (2,3-dihydro-1-benzofuran-5-ylmethyl)methanamine |
| 2026. | 1-(1-butyl-2-phenyl-1H-imidazol-5-yl)-N-(2,3-dihydro-1,4-benzodioxin-6-ylmethyl)-N- |
| | (3-ethoxybenzyl)methanamine |
| 2027. | 1-[1-butyl-2-phenyl-4-(trifluoromethyl)-1H-imidazol-5-yl]-N-(2,3-dihydro-1,4- |
| | benzodioxin-6-ylmethyl)-N-(2,3-dihydro-1-benzofuran-5-ylmethyl)methanamine |
| 2028. | 1-[1-butyl-2-phenyl-4-(trifluoromethyl)-1H-imidazol-5-yl]-N-(2,3-dihydro-1,4- |
| | benzodioxin-6-ylmethyl)-N-(3-ethoxybenzyl)methanamine |
| 2029. | 1-(1,3-benzodioxol-5-yl)-N-(1,3-benzodioxol-5-ylmethyl)-N-{[1-butyl-2-(4- |
| | methylphenyl)-4-phenyl-1H-imidazol-5-yl]methyl}methanamine |
| 2030. | 1-(1,3-benzodioxol-5-yl)-N-(1,3-benzodioxol-5-ylmethyl)-N-{[1-butyl-2-(2- |
| | methylphenyl)-4-phenyl-1H-imidazol-5-yl]methyl}methanamine |
| 2031. | 1-(1,3-benzodioxol-5-yl)-N-(1,3-benzodioxol-5-ylmethyl)-N-{[1-butyl-2-(3- |
| | fluorophenyl)-4-phenyl-1H-imidazol-5-yl]methyl}methanamine |
| 2032. | N-(1,3-benzodioxol-5-ylmethyl)-N-[(1-butyl-4-fluoro-2-phenyl-1H-imidazol-5- |
| | yl)methyl]butan-1-amine |
| 2033. | 1-(1,3-benzodioxol-5-yl)-N-[(1-butyl-4-fluoro-2-phenyl-1H-imidazol-5-yl)methyl]-N- |
| | (cyclohexylmethyl)methanamine |
| 2034. | 1-(1,3-benzodioxol-5-yl)-N-benzyl-N-[(1-butyl-4-fluoro-2-phenyl-1H-imidazol-5- |
| | yl)methyl]methanamine |
| 2035. | N-[(1-butyl-4-fluoro-2-phenyl-1H-imidazol-5-yl)methyl]-N-(cyclohexylmethyl)-N-(1H- |
| | indol-5-ylmethyl)amine |
| 2036. | 1-(1,3-benzodioxol-5-yl)-N-[(1-butyl-2,4-diphenyl-1H-imidazol-5-yl)methyl]-N- |
| | (cyclohexylmethyl)methanamine |
| 2037. | N-{[1-butyl-4-phenyl-2-(1H-pyrazol-1-yl)-1H-imidazol-5-yl]methyl}-N-(2,3-dihydro- |
| | 1,4-benzodioxin-6-ylmethyl)-N-(3-ethoxybenzyl)amine |
| 2038. | 4-(2-{(1,3-benzodioxol-5-ylmethyl)[(1-butyl-2,4-diphenyl-1H-imidazol-5- |
| | yl)methyl]amino}ethyl)phenol |
| 2039. | methyl 2-amino-4-[((1,3-benzodioxol-5-ylmethyl){[1-butyl-2-phenyl-4- |
| | (trifluoromethyl)-1H-imidazol-5-yl]methyl}amino)methyl]benzoate |
| 2040. | 7-[((1,3-benzodioxol-5-ylmethyl){[1-butyl-2-phenyl-4-(trifluoromethyl)-1H-imidazol- |
| | 5-yl]methyl}amino)methyl]quinazolin-4-ol |
| 2041. | methyl 4-({(1,3-benzodioxol-5-ylmethyl)[(1-butyl-2,4-diphenyl-1H-imidazol-5- |
| | yl)methyl]amino}methyl)-2-nitrobenzoate |
| 2042. | 1-(1,3-benzodioxol-5-yl)-N-(1,3-benzodioxol-5-ylmethyl)-N-{[1-butyl-4-(4- |
| | fluorophenyl)-2-phenyl-1H-imidazol-5-yl]methyl}methanamine |
| 2043. | 7-({(1,3-benzodioxol-5-ylmethyl)[(1-butyl-2,4-diphenyl-1H-imidazol-5- |
| | yl)methyl]amino}methyl)quinazolin-4-ol |
| 2044. | 7-({(1,3-benzodioxol-5-ylmethyl)[(1-butyl-2,4-diphenyl-1H-imidazol-5- |
| | yl)methyl]amino}methyl)quinazolin-4-amine |
| 2045. | 7-({(1,3-benzodioxol-5-ylmethyl)[(1-butyl-2,4-diphenyl-1H-imidazol-5- |
| | yl)methyl]amino}methyl)-N,N-diethylquinazolin-4-amine |
| 2046. | 7-({(1,3-benzodioxol-5-ylmethyl)[(1-butyl-2,4-diphenyl-1H-imidazol-5- |
| | yl)methyl]amino}methyl)-N-ethylquinazolin-4-amine |
| 2047. | 1-(1,3-benzodioxol-5-yl)-N-[(1-butyl-2,4-diphenyl-1H-imidazol-5-yl)methyl]-N-[(4- |
| | ethoxyquinazolin-7-yl)methyl]methanamine |
| 2048. | 1-(1-butyl-2,4-diphenyl-1H-imidazol-5-yl)-N-(cyclohexylmethyl)-N- |
| | methylmethanamine |
| 2049. | N-[(1-butyl-2,4-diphenyl-1H-imidazol-5-yl)methyl]-N-(cyclohexylmethyl)butan-1- |
| | amine |
| 2050. | N-[(1-butyl-2,4-diphenyl-1H-imidazol-5-yl)methyl]-1-cyclohexyl-N-methylethanamine |
| 2051. | N-[(1-butyl-4-chloro-2-phenyl-1H-imidazol-5-yl)methyl]-N-(2,3-dihydro-1,4- |
| | benzodioxin-6-ylmethyl)butan-1-amine |
| 2052. | N-[(1-butyl-4-chloro-2-phenyl-1H-imidazol-5-yl)methyl]-N-[4- |
| | (difluoromethoxy)benzyl]butan-1-amine |
| 2053. | N-[(1-butyl-4-chloro-2-phenyl-1H-imidazol-5-yl)methyl]-N-(3-ethoxybenzyl)butan-1- |
| | amine |
| 2054. | N-[(1-butyl-4-chloro-2-phenyl-1H-imidazol-5-yl)methyl]-N-(3-methoxybenzyl)butan- |
| | 1-amine |
| 2055. | 1-(1,3-benzodioxol-5-yl)-N-(1,3-benzodioxol-5-ylmethyl)-N-{[1-butyl-2-phenyl-4- |
| | (phenylsulfonyl)-1H-imidazol-5-yl]methyl}methanamine |
| 2056. | 1-(1,3-benzodioxol-5-yl)-N-[(1-butyl-4-fluoro-2-phenyl-1H-imidazol-5-yl)methyl]-N- |
| | (3-ethoxybenzyl)methanamine |
| 2057. | 1-(1,3-benzodioxol-5-yl)-N-(1,3-benzodioxol-5-ylmethyl)-N-[(1-butyl-4-fluoro-2- |
| | phenyl-1H-imidazol-5-yl)methyl]methanamine |
| 2058. | 1-(1,3-benzodioxol-5-yl)-N-[(1-butyl-2,4-diphenyl-1H-imidazol-5-yl)methyl]-N- |
| | methylmethanamine |
| 2059. | N-(1,3-benzodioxol-5-ylmethyl)-N-[(1-butyl-2,4-diphenyl-1H-imidazol-5- |
| | yl)methyl]ethanamine |
| 2060. | N-(1,3-benzodioxol-5-ylmethyl)-N-[(1-butyl-2,4-diphenyl-1H-imidazol-5- |
| | yl)methyl]propan-1-amine |
| 2061. | N-(1,3-benzodioxol-5-ylmethyl)-N-[(1-butyl-2,4-diphenyl-1H-imidazol-5-yl)methyl]-3- |
| | ethoxypropan-1-amine |
| 2062. | 1-(1-butyl-2,4-diphenyl-1H-imidazol-5-yl)-N-(2,3-dihydro-1,4-benzodioxin-6- |
| | ylmethyl)-N-(2,3-dihydro-1-benzofuran-5-ylmethyl)methanamine |
| 2063. | 1-(1-butyl-2,4-diphenyl-1H-imidazol-5-yl)-N-(2,3-dihydro-1,4-benzodioxin-6- |
| | ylmethyl)-N-(3-ethoxybenzyl)methanamine |
| 2064. | 1-(1,3-benzodioxol-5-yl)-N-[(1-butyl-4-chloro-2-phenyl-1H-imidazol-5-yl)methyl]-N- |
| | methylmethanamine |
| 2065. | N-(1,3-benzodioxol-5-ylmethyl)-N-[(1-butyl-4-chloro-2-phenyl-1H-imidazol-5- |
| | yl)methyl]ethanamine |
| 2066. | N-(1,3-benzodioxol-5-ylmethyl)-N-[(1-butyl-4-chloro-2-phenyl-1H-imidazol-5- |
| | yl)methyl]propan-1-amine |
| 2067. | N-(1,3-benzodioxol-5-ylmethyl)-N-[(1-butyl-4-chloro-2-phenyl-1H-imidazol-5- |
| | yl)methyl]-3-ethoxypropan-1-amine |
| 2068. | N-[(1-butyl-4-chloro-2-phenyl-1H-imidazol-5-yl)methyl]-N-(2,3-dihydro-1,4- |
| | benzodioxin-6-ylmethyl)-N-(2,3-dihydro-1-benzofuran-5-ylmethyl)amine |
| 2069. | N-[(1-butyl-4-chloro-2-phenyl-1H-imidazol-5-yl)methyl]-N-(2,3-dihydro-1,4- |
| | benzodioxin-6-ylmethyl)-N-(3-ethoxybenzyl)amine |
| 2070. | 1-(1,3-benzodioxol-5-yl)-N-{[1-butyl-4-phenyl-2-(1H-pyrazol-1-yl)-1H-imidazol-5- |
| | yl]methyl}-N-(3-ethoxybenzyl)methanamine |
| 2071. | 1-(1,3-benzodioxol-5-yl)-N-(1,3-benzodioxol-5-ylmethyl)-N-{[1-butyl-2-(2,6- |
| | dimethylphenyl)-4-phenyl-1H-imidazol-5-yl]methyl}methanamine |
| 2072. | N-[(1-butyl-4-chloro-2-phenyl-1H-imidazol-5-yl)methyl]-N-(2,3-dihydro-1- |
| | benzofuran-5-ylmethyl)butan-1-amine |
| 2073. | N-[(1-butyl-4-chloro-2-phenyl-1H-imidazol-5-yl)methyl]-N-[3- |
| | (difluoromethoxy)benzyl]butan-1-amine |
| 2074. | 1-(1,3-benzodioxol-5-yl)-N-(1,3-benzodioxol-5-ylmethyl)-N-[(5-butyl-1-phenyl-1H- |
| | pyrazol-4-yl)methyl]methanamine |
| 2075. | N-(1,3-benzodioxol-5-ylmethyl)-N-[(5-butyl-1-phenyl-1H-pyrazol-4-yl)methyl]butan- |
| | 1-amine |
| 2076. | 1-(1,3-benzodioxol-5-yl)-N-[(1-butyl-2,4-diphenyl-1H-imidazol-5-yl)methyl]-N-[(1,1- |
| | dioxidotetrahydro-2H-thiopyran-4-yl)methyl]methanamine |
| 2077. | N-(1,3-benzodioxol-5-ylmethyl)-N-[(1-butyl-2,4-diphenyl-1H-imidazol-5- |
| | yl)methyl]methanesulfonamide |
| 2078. | N-[1-(1,3-benzodioxol-5-yl)ethyl]-N-[(1-butyl-2,4-diphenyl-1H-imidazol-5- |
| | yl)methyl]butan-1-amine |
| 2079. | N-[(1-butyl-2,4-diphenyl-1H-imidazol-5-yl)methyl]-N-[1-(3-ethoxyphenyl)ethyl]butan- |
| | 1-amine |
| 2080. | N-[(1-butyl-2,4-diphenyl-1H-imidazol-5-yl)methyl]-N-{1-[4- |
| | (difluoromethoxy)phenyl]ethyl}butan-1-amine |
| 2081. | N-[(1-butyl-2,4-diphenyl-1H-imidazol-5-yl)methyl]-N-[1-(2,3-dihydro-1,4- |
| | benzodioxin-6-yl)ethyl]butan-1-amine |
| 2082. | 4-(1-{butyl[(1-butyl-2,4-diphenyl-1H-imidazol-5-yl)methyl]amino}ethyl)benzoic acid |
| 2083. | N-{[3-(benzyloxy)isoxazol-5-yl]methyl}-N-[(1-butyl-2,4-diphenyl-1H-imidazol-5- |
| | yl)methyl]butan-1-amine |
| 2084. | 1-(1,3-benzodioxol-5-yl)-N-[(1-butyl-4-chloro-2-phenyl-1H-imidazol-5-yl)methyl]-N- |
| | [(2-methyl-1,3-benzothiazol-5-yl)methyl]methanamine |
| 2085. | N-[(1-butyl-4-chloro-2-phenyl-1H-imidazol-5-yl)methyl]-N-[(2-methyl-1,3- |
| | benzothiazol-5-yl)methyl]butan-1-amine |
| 2086. | 1-(1,3-benzodioxol-5-yl)-N-(1,3-benzodioxol-5-ylmethyl)-N-[(5-butyl-3-chloro-6- |
| | phenylpyridazin-4-yl)methyl]methanamine |
| 2087. | N-(1,3-benzodioxol-5-ylmethyl)-N-[(5-butyl-3-chloro-6-phenylpyridazin-4- |
| | yl)methyl]butan-1-amine |
| 2088. | 5-({butyl[(1-butyl-2,4-diphenyl-1H-imidazol-5-yl)methyl]amino}methyl)isoxazol-3-ol |
| 2089. | N-[1-(1,3-benzodioxol-5-yl)ethyl]-N-[(1-butyl-4-chloro-2-phenyl-1H-imidazol-5- |
| | yl)methyl]propan-1-amine |
| 2090. | N-[(1-butyl-4-chloro-2-phenyl-1H-imidazol-5-yl)methyl]-N-[1-(2,3-dihydro-1,4- |
| | benzodioxin-6-yl)ethyl]propan-1-amine |
| 2091. | 1-(1,3-benzodioxol-5-yl)-N-[(5-butyl-3-chloro-6-phenylpyridazin-4-yl)methyl]-N-(3- |
| | ethoxybenzyl)methanamine |
| 2092. | 4-[((1,3-benzodioxol-5-ylmethyl){[1-butyl-4-(4-methoxyphenyl)-2-phenyl-1H- |
| | imidazol-5-yl]methyl}amino)methyl]benzamide |
| 2093. | 1-(1,3-benzodioxol-5-yl)-N-(1,3-benzodioxol-5-ylmethyl)-N-[(1-butyl-2,4-diphenyl- |
| | 1H-imidazol-5-yl)methyl]methanamine |
| 2094. | 4-({benzyl[(1-butyl-2,4-diphenyl-1H-imidazol-5-yl)methyl]amino}methyl)-2,6- |
| | dichlorophenol |
| 2095. | 4-({benzyl[(1-butyl-2,4-diphenyl-1H-imidazol-5-yl)methyl]amino}methyl)-2- |
| | chlorophenol |
| 2096. | 4-({(1,3-benzodioxol-5-ylmethyl)[1-(1-butyl-2,4-diphenyl-1H-imidazol-5- |
| | yl)ethyl]amino}methyl)-2,6-dimethylphenol |
| 2097. | N-[(1-butyl-2,4-diphenyl-1H-imidazol-5-yl)methyl]-N-(3,4-dichlorobenzyl)butan-1- |
| | amine |
| 2098. | (1R)-N-(1,3-benzodioxol-5-ylmethyl)-1-(1-butyl-2,4-diphenyl-1H-imidazol-5-yl)-N- |
| | (cyclohexylmethyl)pentan-1-amine |
| 2099. | (1R)-N,N-bis(1,3-benzodioxol-5-ylmethyl)-1-(1-butyl-2,4-diphenyl-1H-imidazol-5- |
| | yl)pentan-1-amine |
| 2100. | (1R)-1-(1-butyl-2,4-diphenyl-1H-imidazol-5-yl)-N-(cyclohexylmethyl)-N-(2,3- |
| | dihydro-1-benzofuran-6-ylmethyl)pentan-1-amine |
| 2101. | (1R)-N-(1,3-benzodioxol-5-ylmethyl)-1-(1-butyl-2,4-diphenyl-1H-imidazol-5-yl)-N- |
| | (2,3-dihydro-1-benzofuran-6-ylmethyl)pentan-1-amine |
| 2102. | 4-{[[(1R)-1-(1-butyl-2,4-diphenyl-1H-imidazol-5- |
| | yl)pentyl](cyclohexylmethyl)amino]methyl}-2,6-dimethylphenol |
| 2103. | 4-({(1,3-benzodioxol-5-ylmethyl)[(1R)-1-(1-butyl-2,4-diphenyl-1H-imidazol-5- |
| | yl)pentyl]amino}methyl)-2,6-dimethylphenol |
| 2104. | (1R)-N-(1,3-benzodioxol-5-ylmethyl)-1-(1-butyl-2,4-diphenyl-1H-imidazol-5-y1)-N- |
| | (cyclohexylmethyl)ethanamine |
| 2105. | (1R)-N,N-bis(1,3-benzodioxol-5-ylmethyl)-1-(1-butyl-2,4-diphenyl-1H-imidazol-5- |
| | yl)ethanamine |
| 2106. | (1S)-N,N-bis(1,3-benzodioxol-5-ylmethyl)-1-(1-butyl-2,4-diphenyl-1H-imidazol-5- |
| | yl)ethanamine |
| 2107. | (1R)-1-(1-butyl-2,4-diphenyl-1H-imidazol-5-yl)-N-(cyclohexylmethyl)-N-(2,3-dihydro- |
| | 1-benzofuran-6-ylmethyl)ethanamine |
| 2108. | (1R)-N-(1,3-benzodioxol-5-ylmethyl)-1-(1-butyl-2,4-diphenyl-1H-imidazol-5-yl)-N- |
| | (2,3-dihydro-1-benzofuran-6-ylmethyl)ethanamine |
| 2109. | (1S)-1-(1-butyl-2,4-diphenyl-1H-imidazol-5-yl)-N-(cyclohexylmethyl)-N-(2,3-dihydro- |
| | 1-benzofuran-6-ylmethyl)ethanamine |
| 2110. | 4-{[[(1R)-1-(1-butyl-2,4-diphenyl-1H-imidazol-5- |
| | yl)ethyl](cyclohexylmethyl)amino]methyl}-2,6-dimethylphenol |
| 2111. | 4-{[[(1S)-1-(1-butyl-2,4-diphenyl-1H-imidazol-5- |
| | yl)ethyl](cyclohexylmethyl)amino]methyl}-2,6-dimethylphenol |
| 2112. | N-{(1-butyl-2,4-diphenyl-1H-imidazol-5-yl)methyl]-N-(3-fluoro-4- |
| | methoxybenzyl)butan-1-amine |
| 2113. | N-benzyl-1-(1-butyl-2,4-diphenyl-1H-imidazol-5-yl)-N-(3-fluoro-4- |
| | methoxybenzyl)methanamine |
| 2114. | N-[(1-butyl-2,4-diphenyl-1H-imidazol-5-yl)methyl]-N-(4-methoxy-3- |
| | methylbenzyl)butan-1-amine |
| 2115. | N-benzyl-1-(1-butyl-2,4-diphenyl-1H-imidazol-5-yl)-N-(4-methoxy-3- |
| | methylbenzyl)methanamine |
| 2116. | N-[(1-butyl-2,4-diphenyl-1H-imidazol-5-yl)methyl]-N-(3-chloro-4-fluorobenzyl)butan- |
| | 1-amine |
| 2117. | N-benzyl-1-(1-butyl-2,4-diphenyl-1H-imidazol-5-yl)-N-(3-chloro-4- |
| | fluorobenzyl)methanamine |
| 2118. | methyl 4-({butyl[1-(1-butyl-2-phenyl-1H-imidazol-5-yl)ethyl]amino}methyl)benzoate |
| 2119. | methyl 4-({benzyl[1-(1-butyl-2-phenyl-1H-imidazol-5-yl)ethyl]amino}methyl)benzoate |
| 2120. | methyl 4-({butyl[-(1-butyl-2-phenyl-1H-imidazol-5-yl)pentyl]amino}methyl)benzoate |
| 2121. | 5-({benzyl[(1-butyl-2,4-diphenyl-1H-imidazol-5-yl)methyl]amino}methyl)-2- |
| | methoxyphenol |
| 2122. | 4-({butyl[(1-butyl-2,4-diphenyl-1H-imidazol-5-yl)methyl]amino}methyl)-2- |
| | methoxyphenol |
| 2123. | 4-({benzyl[(1-butyl-2,4-diphenyl-1H-imidazol-5-yl)methyl]amino}methyl)-2- |
| | methoxyphenol |
| 2124. | 4-({[(1-butyl-2,4-diphenyl-1H-imidazol-5-yl)methyl]amino}methyl)benzene-1,2-diol |
| 2125. | 1-(1-butyl-2,4-diphenyl-1H-imidazol-5-yl)-N,N-bis(4-methoxybenzyl)methanamine |
| 2126. | 1-(1-butyl-2,4-diphenyl-1H-imidazol-5-yl)-N,N-bis(3,4-dihydroxybenzyl)methanamine |
| 2127. | N-[(1-butyl-2,4-diphenyl-1H-imidazol-5-yl)methyl-N-[2-fluoro-5- |
| | (trifluoromethyl)benzyl]butan-1-amine |
| 2128. | N-[(1-butyl-2,4-diphenyl-1H-imidazol-5-yl)methyl]-N-(4-methoxy-3,5- |
| | dimethylbenzyl)butan-1-amine |
| 2129. | N-[(1-butyl-2,4-diphenyl-1H-imidazol-5-yl)methyl]-N-(3,5-dichloro-4- |
| | methoxybenzyl)butan-1-amine |
| 2130. | N-[(1-butyl-2,4-diphenyl-1H-imidazol-5-yl)methyl]-N-(3-chloro-4- |
| | methoxybenzyl)butan-1-amine |
| 2131. | N-benzyl-1-(1-butyl-2,4-diphenyl-1H-imidazol-5-yl)-N-(4-methoxy-3,5- |
| | dimethylbenzyl)methanamine |
| 2132. | 4-({butyl[(1-butyl-2-phenyl-1H-imidazol-5-yl)methyl]amino}methyl)benzamide |
| 2133. | N-benzyl-1-(1-butyl-2,4-diphenyl-1H-imidazol-5-yl)-N-(3,5-dichloro-4- |
| | methoxybenzyl)methanamine |
| 2134. | N-benzyl-1-(1-butyl-2,4-diphenyl-1H-imidazol-5-yl)-N-(3-chloro-4- |
| | methoxybenzyl)methanamine |
| 2135. | 4-{[[(1-butyl-2,4-diphenyl-1H-imidazol-5-yl)methyl](2,3-dihydro-1-benzofuran-5- |
| | ylmethyl)amino]methyl}benzenesulfonamide |
| 2136. | N-(1,3-benzodioxol-5-ylmethyl)-N-[(1S)-1-(1-butyl-2,4-diphenyl-1H-imidazol-5- |
| | yl)ethyl]butan-1-amine |
| 2137. | N-[(1S)-1-(1-butyl-2,4-diphenyl-1H-imidazol-5-yl)ethyl]-N-(2,3-dihydro-1,4- |
| | benzodioxin-6-ylmethyl)butan-1-amine |
| 2138. | 4-({butyl[(1S)-1-(1-butyl-2,4-diphenyl-1H-imidazol-5-yl)ethyl]amino}methyl)benzoic |
| | acid |
| 2139. | N-(1,3-benzodioxol-5-ylmethyl)-N-[(1R)-1-(1-butyl-2,4-diphenyl-1H-imidazol-5- |
| | yl)ethyl]butan-1-amine |
| 2140. | N-[(1R)-1-(1-butyl-2,4-diphenyl-1H-imidazol-5-yl)ethyl]-N-(2,3-dihydro-1,4- |
| | benzodioxin-6-ylmethyl)butan-1-amine |
| 2141. | 4-({butyl[(1R)-1-(1-butyl-2,4-diphenyl-1H-imidazol-5-yl)ethyl]amino}methyl)benzoic |
| | acid |
| 2142. | methyl 4-({butyl[(1S)-1-(1-butyl-2,4-diphenyl-1H-imidazol-5- |
| | yl)ethyl]amino}methyl)benzoate |
| 2143. | methyl 4-({butyl[(1R)-1-(1-butyl-2,4-diphenyl-1H-imidazol-5- |
| | yl)ethyl]amino}methyl)benzoate |
| 2144. | 4-({butyl[(1S)-1-(1-butyl-2,4-diphenyl-1H-imidazol-5- |
| | yl)ethyl]amino}methyl)benzamide |
| 2145. | 4-({butyl[(1R)-1-(1-butyl-2,4-diphenyl-1H-imidazol-5- |
| | yl)ethyl]amino}methyl)benzamide |
| 2146. | 4-({butyl[(1-butyl-2,4-diphenyl-1H-imidazol-5-yl)methyl]amino}methyl)-2,6- |
| | dichlorophenol |
| 2147. | 5-({butyl[(1-butyl-2,4-diphenyl-1H-imidazol-5-yl)methyl]amino}methyl)-2- |
| | methoxyphenol |
| 2148. | 4-({benzyl[(1-butyl-2,4-diphenyl-1H-imidazol-5-yl)methyl]amino}methyl)-2- |
| | methylphenol |
| 2149. | 4-({butyl[(1-butyl-2,4-diphenyl-1H-imidazol-5-yl)methyl]amino}methyl)-2- |
| | methylphenol |
| 2150. | 4-({butyl[(1-butyl-2,4-diphenyl-1H-imidazol-5-yl)methyl]amino}methyl)-2- |
| | chlorophenol |
| 2151. | N-benzyl-1-(1-butyl-2,4-diphenyl-1H-imidazol-5-yl)-N-[2-fluoro-3- |
| | (trifluoromethyl)benzyl]methanamine |
| 2152. | N-[(1-butyl-2,4-diphenyl-1H-imidazol-5-yl)methyl]-N-[2-fluoro-3- |
| | (trifluoromethyl)benzyl]butan-1-amine |
| 2153. | 1-(1-butyl-2,4-diphenyl-1H-imidazol-5-yl)-N-(cyclohexylmethyl)-N-[2-fluoro-3- |
| | (trifluoromethyl)benzyl]methanamine |
| 2154. | 1-(1,3-benzodioxol-5-yl)-N-[(1-butyl-2,4-diphenyl-1H-imidazol-5-yl)methyl]-N-[2- |
| | fluoro-3-(trifluoromethyl)benzyl]methanamine |
| 2155. | N-benzyl-1-(1-butyl-2,4-diphenyl-1H-imidazol-5-yl)-N-[2-fluoro-4- |
| | (trifluoromethyl)benzyl]methanamine |
| 2156. | N-[(1-butyl-2,4-diphenyl-1H-imidazol-5-yl)methyl]-N-[2-fluoro-4- |
| | (trifluoromethyl)benzyl]butan-1-amine |
| 2157. | 1-(1-butyl-2,4-diphenyl-1H-imidazol-5-yl)-N-(cyclohexylmethyl)-N-[2-fluoro-4- |
| | (trifluoromethyl )benzyl]methanamine |
| 2158. | 1-(1,3-benzodioxol-5-yl)-N-[(1-butyl-2,4-diphenyl-1H-imidazol-5-yl)methyl]-N-[2- |
| | fluoro-4-(trifluoromethyl)benzyl]methanamine |
| 2159. | N-benzyl-1-(1-butyl-2,4-diphenyl-1H-imidazol-5-yl)-N-[2-fluoro-5- |
| | (trifluoromethyl)benzyl]methanamine |
| 2160. | 1-(1-butyl-2,4-diphenyl-1H-imidazol-5-yl)-N-(cyclohexylmethyl)-N-[2-fluoro-5- |
| | (trifluoromethyl)benzyl]methanamine |
| 2161. | 1-(1,3-benzodioxol-5-yl)-N-[(1-butyl-2,4-diphenyl-1H-imidazol-5-yl)methyl]-N-[2- |
| | fluoro-5-(trifluoromethyl)benzyl]methanamine |
| 2162. | N-benzyl-1-(1-butyl-2,4-diphenyl-1H-imidazol-5-yl)-N-[4- |
| | (difluoromethoxy)benzyl]methanamine |
| 2163. | N-[(1-butyl-2,4-diphenyl-1H-imidazol-5-yl)methyl]-N-[4- |
| | (difluoromethoxy)benzyl]butan-1-amine |
| 2164. | 1-(1-butyl-2,4-diphenyl-1H-imidazol-5-yl)-N-(cyclohexylmethyl)-N-[4- |
| | (difluoromethoxy)benzyl]methanamine |
| 2165. | 1-(1,3-benzodioxol-5-yl)-N-[(1-butyl-2,4-diphenyl-1H-imidazol-5-yl)methyl]-N-[4- |
| | (difluoromethoxy)benzyl]methanamine |
| 2166. | N-benzyl-1-(1-butyl-2,4-diphenyl-1H-imidazol-5-yl)-N-[3-fluoro-4- |
| | (trifluoromethyl)benzyl]methanamine |
| 2167. | N-[(1-butyl-2,4-diphenyl-1H-imidazol-5-yl)methyl]-N-[3-fluoro-4- |
| | (trifluoromethyl)benzyl]butan-1-amine |
| 2168. | 1-(1-butyl-2,4-diphenyl-1H-imidazol-5-yl)-N-(cyclohexylmethyl)-N-[3-fluoro-4- |
| | (trifluoromethyl)benzyl]methanamine |
| 2169. | 1-(1,3-benzodioxol-5-yl)-N-[(1-butyl-2,4-diphenyl-1H-imidazol-5-yl)methyl]-N-[3- |
| | fluoro-4-(trifluoromethyl)benzyl]methanamine |
| 2170. | N-benzyl-1-(1-butyl-2,4-diphenyl-1H-imidazol-5-yl)-N-[3- |
| | (trifluoromethyl)benzyl]methanamine |
| 2171. | N-[(1-butyl-2,4-diphenyl-1H-imidazol-5-yl)methyl]-N-[3- |
| | (trifluoromethyl)benzyl]butan-1-amine |
| 2172. | 1-(1-butyl-2,4-diphenyl-1H-imidazol-5-yl)-N-(cyclohexylmethyl )-N-[3- |
| | (trifluoromethyl)benzyl]methanamine |
| 2173. | 1-(1,3-benzodioxol-5-yl)-N-[(1-butyl-2,4-diphenyl-1H-imidazol-5-yl)methyl]-N-[3- |
| | (trifluoromethyl)benzyl]methanamine |
| 2174. | N-benzyl-1-(1-butyl-2,4-diphenyl-1H-imidazol-5-yl)-N-[4- |
| | (methylthio)benzyl]methanamine |
| 2175. | N-[(1-butyl-2,4-diphenyl-1H-imidazol-5-yl)methyl]-N-[4-(methylthio)benzyl]butan-1- |
| | amine |
| 2176. | N-benzyl-1-(1-butyl-2,4-diphenyl-1H-imidazol-5-yl)-N-[(2,2-dimethyl-3,4-dihydro-2H- |
| | chromen-6-yl)methyl]methanamine |
| 2177. | N-[(1-butyl-2,4-diphenyl-1H-imidazol-5-yl)methyl]-N-[(2,2-dimethyl-3,4-dihydro-2H- |
| | chromen-6-yl)methyl]butan-1-amine |
| 2178. | N-benzyl-1-(1-butyl-2,4-diphenyl-1H-imidazol-5-yl)-N-(4- |
| | isopropylbenzyl)methanamine |
| 2179. | N-[(1-butyl-2,4-diphenyl-1H-imidazol-5-yl)methyl]-N-(4-isopropylbenzyl)butan-1- |
| | amine |
| 2180. | 2-{[1-(2,3-dihydro-1H-inden-2-yl)-2-phenyl-1H-imidazol-5-yl]methyl}-1-(2- |
| | methylphenyl)-1,2,3,4-tetrahydroisoquinoline |
| 2181. | 1-(1,3-benzodioxol-5-yl)-N-[(1-butyl-2,4-diphenyl-1H-imidazol-5-yl)methyl]-N-[4- |
| | fluoro-3-(trifluoromethyl)benzyl]methanamine |
| 2182. | N-[(1-butyl-2,4-diphenyl-1H-imidazol-5-yl)methyl]-N-[3-fluoro-5- |
| | (trifluoromethyl)benzyl]butan-1-amine |
| 2183. | 1-(1-butyl-2,4-diphenyl-1H-imidazol-5-yl)-N-(cyclohexylmethyl)-N-[3-fluoro-5- |
| | (trifluoromethyl)benzyl]methanamine |
| 2184. | 1-(1,3-benzodioxol-5-yl)-N-[(1-butyl-2,4-diphenyl-1H-imidazol-5-yl)methyl]-N-[3- |
| | fluoro-5-(trifluoromethyl)benzyl]methanamine |
| 2185. | N-benzyl-1-(1-butyl-2,4-diphenyl-1H-imidazol-5-yl)-N-[4-chloro-3- |
| | (trifluoromethyl)benzyl]methanamine |
| 2186. | N-[(1-butyl-2,4-diphenyl-1H-imidazol-5-yl)methyl]-N-[4-chloro-3- |
| | (trifluoromethyl)benzyl]butan-1-amine |
| 2187. | 1-(1-butyl-2,4-diphenyl-1H-imidazol-5-yl)-N-[4-chloro-3-(trifluoromethyl)benzyl]-N- |
| | (cyclohexylmethyl)methanamine |
| 2188. | 1-(1,3-benzodioxol-5-yl)-N-[(1-butyl-2,4-diphenyl-1H-imidazol-5-yl)methyl]-N-[4- |
| | chloro-3-(trifluoromethyl)benzyl]methanamine |
| 2189. | N-[(1-butyl-2,4-diphenyl-1H-imidazol-5-yl)methyl]-N-[2-chloro-3- |
| | (trifluoromethyl)benzyl]butan-1-amine |
| 2190. | 1-(1-butyl-2,4-diphenyl-1H-imidazol-5-yl)-N-[2-chloro-3-(trifluoromethyl)benzyl]-N- |
| | (cyclohexylmethyl)methanamine |
| 2191. | 1-(1,3-benzodioxol-5-yl)-N-[(1-butyl-2,4-diphenyl-1H-imidazol-5-yl)methyl]-N-[2- |
| | chloro-3-(trifluoromethyl)benzyl]methanamine |
| 2192. | N-benzyl-1-(1-butyl-2,4-diphenyl-1H-imidazol-5-yl)-N-[2-chloro-5- |
| | (trifluoromethyl)benzyl]methanamine |
| 2193. | N-[(1-butyl-2,4-diphenyl-1H-imidazol-5-yl)methyl]-N-[2-chloro-5- |
| | (trifluoromethyl)benzyl]butan-1-amine |
| 2194. | 1-(1-butyl-2,4-diphenyl-1H-imidazol-5-yl)-N-[2-chloro-5-(trifluoromethyl)benzyl]-N- |
| | (cyclohexylmethyl)methanamine |
| 2195. | 1-(1,3-benzodioxol-5-yl)-N-[(1-butyl-2,4-diphenyl-1H-imidazol-5-yl)methyl]-N-[2- |
| | chloro-5-(trifluoromethyl )benzyl]methanamine |
| 2196. | N-benzyl-1-(1-butyl-2,4-diphenyl-1H-imidazol-5-yl)-N-[2,3-difluoro-4- |
| | (trifluoromethyl)benzyl]methanamine |
| 2197. | N-[(1-butyl-2,4-diphenyl-1H-imidazol-5-yl)methyl]-N-[2,3-difluoro-4- |
| | (trifluoromethyl)benzyl]butan-1-amine |
| 2198. | 1-(1-butyl-2,4-diphenyl-1H-imidazol-5-yl)-N-(cyclohexylmethyl)-N-[2,3-difluoro-4- |
| | (trifluoromethyl)benzyl]methanamine |
| 2199. | 1-(1,3-benzodioxol-5-yl)-N-[(1-butyl-2,4-diphenyl-1H-imidazol-5-yl)methyl]-N-[2,3- |
| | difluoro-4-(trifluoromethyl)benzyl]methanamine |
| 2200. | N-benzyl-1-[2,4-bis(trifluoromethyl)phenyl]-N-[(1-butyl-2,4-diphenyl-1H-imidazol-5- |
| | yl)methyl]methanamine |
| 2201. | N-[2,4-bis(trifluoromethyl)benzyl]-N-[(1-butyl-2,4-diphenyl-1H-imidazol-5- |
| | yl)methyl]butan-1-amine |
| 2202. | 1-[2,4-bis(trifluoromethyl)phenyl]-N-[(1-butyl-2,4-diphenyl-1H-imidazol-5-yl)methyl]- |
| | N-(cyclohexylmethyl)methanamine |
| 2203. | N-benzyl-1-[2,5-bis(trifluoromethyl)phenyl]-N-[(1-butyl-2,4-diphenyl-1H-imidazol-5- |
| | yl)methyl]methanamine |
| 2204. | 1-(1,3-benzodioxol-5-yl)-N-[2,5-bis(trifluoromethyl)benzyl]-N-[(1-butyl-2,4-diphenyl- |
| | 1H-imidazol-5-yl)methyl]methanamine |
| 2205. | N-[3,5-bis(trifluoromethyl)benzyl]-N-[(1-butyl-2,4-diphenyl-1H-imidazol-5- |
| | yl)methyl]butan-1-amine |
| 2206. | 1-[3,5-bis(trifluoromethyl)phenyl]-N-[(1-butyl-2,4-diphenyl-1H-imidazol-5-yl)methyl]- |
| | N-(cyclohexylmethyl)methanamine |
| 2207. | 1-(1,3-benzodioxol-5-yl)-N-[3,5-bis(trifluoromethyl)benzyl]-N-[(1-butyl-2,4-diphenyl- |
| | 1H-imidazol-5-yl)methyl]methanamine |
| 2208. | 4-({benzyl[(1-butyl-2,4-diphenyl-1H-imidazol-5-yl)methyl]amino}methyl)-2- |
| | methylphenyl acetate |
| 2209. | 4-({butyl[(1-butyl-2,4-diphenyl-1H-imidazol-5-yl)methyl]amino}methyl)-2- |
| | methylphenyl acetate |
| 2210. | 4-({benzyl[(1-butyl-2,4-diphenyl-1H-imidazol-5-yl)methyl]amino}methyl)-2,6- |
| | dimethylphenyl acetate |
| 2211. | 4-({butyl[(1-butyl-2,4-diphenyl-1H-imidazol-5-yl)methyl]amino}methyl)-2,6- |
| | dimethylphenyl acetate |
| 2212. | 1-(1-butyl-2,4-diphenyl-1H-imidazol-5-yl)-N-[2-fluoro-4-(trifluoromethyl)benzyl]-N- |
| | (4-methoxybenzyl)methanamine |
| 2213. | N,N-dibenzyl-1-(1-butyl-2,4-diphenyl-1H-imidazol-5-yl)methanamine |
| 2214. | 4-({benzyl[(1-butyl-2,4-diphenyl-1H-imidazol-5-yl)methyl]amino}methyl)-2,6- |
| | dimethylphenyl methanesulfonate |
| 2215. | N-butyl-N-[(1-butyl-2,4-diphenyl-1H-imidazol-5-yl)methyl]butan-1-amine |
| 2216. | 4-({butyl[(1-butyl-2,4-diphenyl-1H-imidazol-5-yl)methyl]amino}methyl)-2,6- |
| | dimethylphenyl methanesulfonate |
| 2217. | 4-({[(1-butyl-2,4-diphenyl-1H-imidazol-5-yl)methyl][4- |
| | (trifluoromethyl)benzyl]amino}methyl)-2,6-dimethylphenol |
| 2218. | 1-(1-butyl-2,4-diphenyl-1H-imidazol-5-yl)-N-[4-(difluoromethoxy)benzyl]-N-[4- |
| | (trifluoromethyl)benzyl]methanamine |
| 2219. | 1-(1-butyl-2,4-diphenyl-1H-imidazol-5-yl)-N-[4-(difluoromethoxy)benzyl]-N-[3- |
| | (trifluoromethyl)benzyl]methanamine |
| 2220. | 1-(1-butyl-2,4-diphenyl-1H-imidazol-5-yl)-N-[4-(trifluoromethoxy)benzyl]-N-[3- |
| | (trifluoromethyl)benzyl]methanamine |
| 2221. | 1-(1-butyl-2,4-diphenyl-1H-imidazol-5-yl)-N,N-bis[4- |
| | (difluoromethoxy)benzyl]methanamine |
| 2222. | 1-(1-butyl-2,4-diphenyl-1H-imidazol-5-yl)-N-[4-(difluoromethoxy)benzyl]-N-[4- |
| | (trifluoromethoxy)benzyl]methanamine |
| 2223. | 4-({[(1-butyl-2,4-diphenyl-1H-imidazol-5-yl)methyl][3- |
| | (trifluoromethyl)benzyl]amino}methyl)-3-chlorophenol |
| 2224. | 1-(1-butyl-2,4-diphenyl-1H-imidazol-5-yl)-N-[4-(difluoromethoxy)benzyl]-N-[2- |
| | fluoro-4-(trifluoromethyl)benzyl]methanamine |
| 2225. | N-benzyl-1-(1-butyl-2,4-diphenyl-1H-imidazol-5-yl)-N-(3,4- |
| | dimethoxybenzyl)methanamine |
| 2226. | N-[(1-butyl-2,4-diphenyl-1H-imidazol-5-yl)methyl]-N-(3,4-dimethoxybenzyl)butan-1- |
| | amine |
| 2227. | 1-(1-butyl-2,4-diphenyl-1H-imidazol-5-yl)-N-(cyclohexylmethyl)-N-(3,4- |
| | dimethoxybenzyl)methanamine |
| 2228. | 1-(1,3-benzodioxol-5-yl)-N-[(1-butyl-2,4-diphenyl-1H-imidazol-5-yl)methyl]-N-(3,4- |
| | dimethoxybenzyl)methanamine |
| 2229. | N-benzyl-1-(1-butyl-2,4-diphenyl-1H-imidazol-5-yl)-N-(3,5- |
| | dimethoxybenzyl)methanamine |
| 2230. | N-[(1-butyl-2,4-diphenyl-1H-imidazol-5-yl)methyl]-N-(3,5-dimethoxybenzyl)butan-1- |
| | amine |
| 2231. | 1-(1-butyl-2,4-diphenyl-1H-imidazol-5-yl)-N-(cyclohexylmethyl)-N-(3,5- |
| | dimethoxybenzyl)methanamine |
| 2232. | 1-(1,3-benzodioxol-5-yl)-N-[(1-butyl-2,4-diphenyl-1H-imidazol-5-yl)methyl]-N-(3,5- |
| | dimethoxybenzyl)methanamine |
| 2233. | N-benzyl-1-(1-butyl-2,4-diphenyl-1H-imidazol-5-yl)-N-(3,4- |
| | diethoxybenzyl)methanamine |
| 2234. | N-[(1-butyl-2,4-diphenyl-1H-imidazol-5-yl)methyl]-N-(3,4-diethoxybenzyl)butan-1- |
| | amine |
| 2235. | 1-(1-butyl-2,4-diphenyl-1H-imidazol-5-yl)-N-(cyclohexylmethyl)-N-(3,4- |
| | diethoxybenzyl)methanamine |
| 2236. | 1-(1,3-benzodioxol-5-yl)-N-[(1-butyl-2,4-diphenyl-1H-imidazol-5-yl)methyl]-N-(3,4- |
| | diethoxybenzyl)methanamine |
| 2237. | 1-(1-butyl-2,4-diphenyl-1H-imidazol-5-yl)-N-[4-(difluoromethoxy)benzyl]-N-(3,4- |
| | dimethoxybenzyl)methanamine |
| 2238. | 1-(1-butyl-2,4-diphenyl-1H-imidazol-5-yl)-N-(3,5-dimethoxybenzyl)-N-[3- |
| | (trifluoromethyl)benzyl]methanamine |
| 2239. | 1-(1-butyl-2,4-diphenyl-1H-imidazol-5-yl)-N-[4-(difluoromethoxy)benzyl]-N-(3,5- |
| | dimethoxybenzyl)methanamine |
| 2240. | 1-(1-butyl-2,4-diphenyl-1H-imidazol-5-yl)-N-(3,4-diethoxybenzyl)-N-[4- |
| | (difluoromethoxy)benzyl]methanamine |
| 2241. | 4-({[(1-butyl-2,4-diphenyl-1H-imidazol-5-yl)methyl][3- |
| | (trifluoromethyl)benzyl]amino}methyl)benzenesulfonamide |
| 2242. | 4-({[(1-butyl-2,4-diphenyl-1H-imidazol-5-yl)methyl][4- |
| | (difluoromethoxy)benzyl]amino}methyl)benzenesulfonamide |
| 2243. | 4-({[(1-butyl-2,4-diphenyl-1H-imidazol-5-yl)methyl][4- |
| | (trifluoromethoxy)benzyl]amino}methyl)benzenesulfonamide |
| 2244. | (1R)-N-(1,3-benzodioxol-5-ylmethyl)-1-(1-butyl-2,4-diphenyl-1H-imidazol-5-yl)-N-[4- |
| | (difluoromethoxy)benzyl]pentan-1-amine |
| 2245. | (1R)-N-(1,3-benzodioxol-5-ylmethyl)-1-(1-butyl-2,4-diphenyl-1H-imidazol-5-yl)-N-[4- |
| | (difluoromethoxy)benzyl]ethanamine |
| 2246. | (1R)-1-(1-butyl-2,4-diphenyl-1H-imidazol-5-yl)-N-(cyclohexylmethyl)-N-[4- |
| | (difluoromethoxy)benzyl]ethanamine |
| 2247. | (1S)-1-(1-butyl-2,4-diphenyl-1H-imidazol-5-yl)-N-(cyclohexylmethyl)-N-[4- |
| | (difluoromethoxy)benzyl]pentan-1-amine |
| 2248. | (1S)-1-(1-butyl-2,4-diphenyl-1H-imidazol-5-yl)-N-(cyclohexylmethyl)-N-[4- |
| | (difluoromethoxy)benzyl]ethanamine |
| 2249. | N-[(1-butyl-2,4-diphenyl-1H-imidazol-5-yl)methyl]-N-[2-chloro-4- |
| | (difluoromethoxy)benzyl]butan-1-amine |
| 2250. | N-benzyl-1-(1-butyl-2,4-diphenyl-1H-imidazol-5-yl)-N-[2-chloro-4- |
| | (difluoromethoxy)benzyl]methanamine |
| 2251. | 4-({[(1-butyl-2,4-diphenyl-1H-imidazol-5-yl)methyl][2-chloro-4- |
| | (difluoromethoxy)benzyl]amino}methyl)benzenesulfonamide |
| 2252. | N-[(1-butyl-2,4-diphenyl-1H-imidazol-5-yl)methyl]-N-[4-(difluoromethoxy)-3- |
| | methylbenzyl]butan-1-amine |
| 2253. | N-benzyl-3-butyl-N-[4-(difluoromethoxy)benzyl]-2-phenyl-3,4,5,6- |
| | tetrahydrocyclopenta[d]imidazol-4-amine |
| 2254. | N-benzyl-3-butyl-2-phenyl-N-[3-(trifluoromethyl)benzyl]-3,4,5,6- |
| | tetrahydrocyclopenta[d]imidazol-4-amine |
| 2255. | N,3-dibutyl-N-[4-(difluoromethoxy)benzyl]-2-phenyl-3,4,5,6- |
| | tetrahydrocyclopenta[d]imidazol-4-amine |
| 2256. | N,3-dibutyl-2-phenyl-N-[3-(trifluoromethyl)benzyl]-3,4,5,6- |
| | tetrahydrocyclopenta[d]imidazol-4-amine |
| 2257. | N-benzyl-1-(1-butyl-2,4-diphenyl-1H-imidazol-5-yl)-N-[4-(difluoromethoxy)-3- |
| | methylbenzyl]methanamine |
| 2258. | N-benzyl-1-(1-butyl-2,4-diphenyl-1H-imidazol-5-yl)-N-[4-(difluoromethoxy)-3,5- |
| | dimethylbenzyl]methanamine |
| 2259. | N-[(1-butyl-2,4-diphenyl-1H-imidazol-5-yl)methyl]-N-[4-(difluoromethoxy)-3,5- |
| | dimethylbenzyl]butan-1-amine |
| 2260. | N,1-dibutyl-5,5-dimethyl-2-phenyl-N-[3-(trifluoromethyl)benzyl]-4,5,6,7-tetrahydro- |
| | 1H-benzimidazol-7-amine |
| 2261. | 1-butyl-N-[4-(difluoromethoxy)benzyl]-5,5-dimethyl-2-phenyl-N-[3- |
| | (trifluoromethyl)benzyl]-4,5,6,7-tetrahydro-1H-benzimidazol-7-amine |
| 2262. | bis(1,3-benzodioxol-5-ylmethyl)(1-butyl-2,4-diphenyl-1H-imidazol-5-yl)methylamine |
| | oxide |
| 2263. | N-[(1-butyl-2,4-diphenyl-1H-imidazol-5-yl)methyl]-N-[4-(difluoromethoxy)-3- |
| | fluorobenzyl]butan-1-amine |
| 2264. | N-benzyl-1-(1-butyl-2,4-diphenyl-1H-imidazol-5-yl)-N-[4-(difluoromethoxy)-3- |
| | fluorobenzyl]methanamine |
| 2265. | 4-({[(1-butyl-2,4-diphenyl-1H-imidazol-5-yl)methyl][4-(difluoromethoxy)-3- |
| | fluorobenzyl]amino}methyl)benzenesulfonamide |
| 2266. | 4-({[(1-butyl-2,4-diphenyl-1H-imidazol-5-yl)methyl][4-(difluoromethoxy)-3,5- |
| | dimethyl benzyl]amino}methyl)benzenesulfonamide |
| 2267. | (1S)-1-(1-butyl-2,4-diphenyl-1H-imidazol-5-yl)-N-(cyclohexylmethyl)-N-[3- |
| | (trifluoromethyl)benzyl]pentan-1-amine |
| 2268. | (1R)-N-(1,3-benzodioxol-5-ylmethyl)-1-(1-butyl-2,4-diphenyl-1H-imidazol-5-yl)-N-[3- |
| | (trifluoromethyl)benzyl]pentan-1-amine |
| 2269. | (1R)-N-(1,3-benzodioxol-5-ylmethyl)-1-(1-butyl-2,4-diphenyl-1H-imidazol-5-yl)-N-[3- |
| | (trifluoromethyl)benzyl]ethanamine |
| 2270. | (1R)-1-(1-butyl-2,4-diphenyl-1H-imidazol-5-yl)-N-(cyclohexylmethyl)-N-[3- |
| | (trifluoromethyl)benzyl]ethanamine |
| 2271. | (1R)-1-(1-butyl-2,4-diphenyl-1H-imidazol-5-yl)-N-(cyclohexylmethyl)-N-[3- |
| | (trifluoromethyl)benzyl]pentan-1-amine |
| 2272. | (1S)-1-(1-butyl-2,4-diphenyl-1H-imidazol-5-yl)-N-(cyclohexylmethyl)-N-[3- |
| | (trifluoromethyl)benzyl]ethanamine |
| 2273. | (1S)-N-(1,3-benzodioxol-5-ylmethyl)-1-(1-butyl-2,4-diphenyl-1H-imidazol-5-yl)-N-[3- |
| | (trifluoromethyl)benzyl]ethanamine |
| 2274. | (1S)-N-(1,3-benzodioxol-5-ylmethyl)-1-(1-butyl-2,4-diphenyl-1H-imidazol-5-yl)-N-[3- |
| | (trifluoromethyl)benzyl]pentan-1-amine |
| 2275. | N-benzyl-1-(1-butyl-2,4-diphenyl-1H-imidazol-5-yl)-N-(4-ethoxybenzyl)methanamine |
| 2276. | N-[(1-butyl-2,4-diphenyl-1H-imidazol-5-yl)methyl]-N-(4-ethoxybenzyl)butan-1-amine |
| 2277. | 1-(1-butyl-2,4-diphenyl-1H-imidazol-5-yl)-N-(cyclohexylmethyl)-N-(4- |
| | ethoxybenzyl)methanamine |
| 2278. | 1-(1,3-benzodioxol-5-yl)-N-[(1-butyl-2,4-diphenyl-1H-imidazol-5-yl)methyl]-N-(4- |
| | ethoxybenzyl)methanamine |
| 2279. | N-benzyl-1-(1-butyl-2,4-diphenyl-1H-imidazol-5-yl)-N-(3-ethoxybenzyl)methanamine |
| 2280. | N-[(1-butyl-2,4-diphenyl-1H-imidazol-5-yl)methyl]-N-(3-ethoxybenzyl)butan-1-amine |
| 2281. | 1-(1-butyl-2,4-diphenyl-1H-imidazol-5-yl)-N-(cyclohexylmethyl)-N-(3- |
| | ethoxybenzyl)methanamine |
| 2282. | 1-(1,3-benzodioxol-5-yl)-N-[(1-butyl-2,4-diphenyl-1H-imidazol-5-yl)methyl]-N-(3- |
| | ethoxybenzyl)methanamine |
| 2283. | N-benzyl-1-(1-butyl-2,4-diphenyl-1H-imidazol-5-yl)-N-[3-(1,1,2,2- |
| | tetrafluoroethoxy)benzyl]methanamine |
| 2284. | N-[(1-butyl-2,4-diphenyl-1H-imidazol-5-yl)methyl]-N-[3-(1,1,2,2- |
| | tetrafluoroethoxy)benzyl]butan-1-amine |
| 2285. | 1-(1-butyl-2,4-diphenyl-1H-imidazol-5-yl)-N-(cyclohexylmethyl)-N-[3-(1,1,2,2- |
| | tetrafluoroethoxy)benzyl]methanamine |
| 2286. | 1-(1,3-benzodioxol-5-yl)-N-[(1-butyl-2,4-diphenyl-1H-imidazol-5-yl)methyl]-N-[3- |
| | (1,1,2,2-tetrafluoroethoxy)benzyl]methanamine |
| 2287. | N-benzyl-1-(1-butyl-2,4-diphenyl-1H-imidazol-5-yl)-N-[3- |
| | (difluoromethoxy)benzyl]methanamine |
| 2288. | N-[(1-butyl-2,4-diphenyl-1H-imidazol-5-yl)methyl]-N-[3- |
| | (difluoromethoxy)benzyl]butan-1-amine |
| 2289. | 1-(1-butyl-2,4-diphenyl-1H-imidazol-5-yl)-N-(cyclohexylmethyl)-N-[3- |
| | (difluoromethoxy)benzyl]methanamine |
| 2290. | 1-(1,3-benzodioxol-5-yl)-N-[(1-butyl-2,4-diphenyl-1H-imidazol-5-yl)methyl]-N-[3- |
| | (difluoromethoxy)benzyl]methanamine |
| 2291. | 1-(1-butyl-2,4-diphenyl-1H-imidazol-5-yl)-N-[3-(difluoromethoxy)benzyl]-N-[3- |
| | (trifluoromethyl)benzyl]methanamine |
| 2292. | 1-(1-butyl-2,4-diphenyl-1H-imidazol-5-yl)-N-[3-(difluoromethoxy)benzyl]-N-[4- |
| | (difluoromethoxy)benzyl]methanamine |
| 2293. | N-benzyl-1-(1-butyl-2,4-diphenyl-1H-imidazol-5-yl)-N-[2- |
| | (difluoromethoxy)benzyl]methanamine |
| 2294. | N-[(1-butyl-2,4-diphenyl-1H-imidazol-5-yl)methyl]-N-[2- |
| | (difluoromethoxy)benzyl]butan-1-amine |
| 2295. | 1-(1-butyl-2,4-diphenyl-1H-imidazol-5-yl)-N-(cyclohexylmethyl)-N-[2- |
| | (difluoromethoxy)benzyl]methanamine |
| 2296. | 1-(1,3-benzodioxol-5-yl)-N-[(1-butyl-2,4-diphenyl-1H-imidazol-5-yl)methyl]-N-[2- |
| | (difluoromethoxy)benzyl]methanamine |
| 2297. | 1-(1-butyl-2,4-diphenyl-1H-imidazol-5-yl)-N-[2-(difluoromethoxy)benzyl]-N-[3- |
| | (trifluoromethyl)benzyl]methanamine |
| 2298. | 1-(1-butyl-2,4-diphenyl-1H-imidazol-5-yl)-N-[2-(difluoromethoxy)benzyl]-N-[4- |
| | (difluoromethoxy)benzyl]methanamine |
| 2299. | 1-(1-butyl-2,4-diphenyl-1H-imidazol-5-yl)-N-(cyclohexylmethyl)-N-[4- |
| | (difluoromethoxy)-3,5-dimethylbenzyl]methanamine |
| 2300. | 1-(1,3-benzodioxol-5-yl)-N-[(1-butyl-2,4-diphenyl-1H-imidazol-5-yl)methyl]-N-[4- |
| | (difluoromethoxy)-3,5-dimethylbenzyl]methanamine |
| 2301. | 1-(1-butyl-2,4-diphenyl-1H-imidazol-5-yl)-N-[4-(difluoromethoxy)-3,5- |
| | dimethylbenzyl]-N-[3-(trifluoromethyl)benzyl]methanamine |
| 2302. | 1-(1-butyl-2,4-diphenyl-1H-imidazol-5-yl)-N-[4-(difluoromethoxy)benzyl]-N-[4- |
| | (difluoromethoxy)-3,5-dimethylbenzyl]methanamine |
| 2303. | 1-(1-butyl-2,4-diphenyl-1H-imidazol-5-yl)-N-(cyclohexylmethyl)-N-[4- |
| | (difluoromethoxy)-3-fluorobenzyl]methanamine |
| 2304. | 1-(1,3-benzodioxol-5-yl)-N-[(1-butyl-2,4-diphenyl-1H-imidazol-5-yl)methyl]-N-[4- |
| | (difluoromethoxy)-3-fluorobenzyl]methanamine |
| 2305. | 1-(1-butyl-2,4-diphenyl-1H-imidazol-5-yl)-N-[4-(difluoromethoxy)-3-fluorobenzyl]-N- |
| | [3-(trifluoromethyl)benzyl]methanamine |
| 2306. | 1-(1-butyl-2,4-diphenyl-1H-imidazol-5-yl)-N-[4-(difluoromethoxy)benzyl]-N-[4- |
| | (difluoromethoxy)-3-fluorobenzyl]methanamine |
| 2307. | 1-(1-butyl-2,4-diphenyl-1H-imidazol-5-yl)-N-[4-(difluoromethoxy)-3-methylbenzyl]- |
| | N-[3-(trifluoromethyl)benzyl]methanamine |
| 2308. | 1-(1-butyl-2,4-diphenyl-1H-imidazol-5-yl)-N-[4-(difluoromethoxy)benzyl]-N-[4- |
| | (difluoromethoxy)-3-methylbenzyl]methanamine |
| 2309. | 1-(1,3-benzodioxol-5-yl)-N-[(1-butyl-2,4-diphenyl-1H-imidazol-5-yl)methyl]-N-(4- |
| | methoxy-3,5-dimethylbenzyl)methanamine |
| 2310. | 1-(1-butyl-2,4-diphenyl-1H-imidazol-5-yl)-N-(4-ethoxybenzyl)-N-[3- |
| | (trifluoromethyl)benzyl]methanamine |
| 2311. | 1-(1-butyl-2,4-diphenyl-1H-imidazol-5-yl)-N-[4-(difluoromethoxy)benzyl]-N-(4- |
| | ethoxybenzyl)methanamine |
| 2312. | 1-(1-butyl-2,4-diphenyl-1H-imidazol-5-yl)-N-(3-ethoxybenzyl)-N-[3- |
| | (trifluoromethyl)benzyl]methanamine |
| 2313. | 1-(1-butyl-2,4-diphenyl-1H-imidazol-5-yl)-N-[4-(difluoromethoxy)benzyl]-N-(3- |
| | ethoxybenzyl)methanamine |
| 2314. | 1-(1-butyl-2,4-diphenyl-1H-imidazol-5-yl)-N-[3-( 1,1,2,2-tetrafluoroethoxy)benzyl]-N- |
| | [3-(trifluoromethyl)benzyl]methanamine |
| 2315. | 1-(1-butyl-2,4-diphenyl-1H-imidazol-5-yl)-N-[4-(difluoromethoxy)benzyl]-N-[3- |
| | (1,1,2,2-tetrafluoroethoxy)benzyl]methanamine |
| 2316. | 1-(1-butyl-2,4-diphenyl-1H-imidazol-5-yl)-N-(4-methoxy-3-methylbenzyl)-N-[3- |
| | (trifluoromethyl)benzyl]methanamine |
| 2317. | 1-(1-butyl-2,4-diphenyl-1H-imidazol-5-yl)-N-[4-(difluoromethoxy)benzyl]-N-(4- |
| | methoxy-3-methylbenzyl)methanamine |
| 2318. | 1-(1-butyl-2,4-diphenyl-1H-imidazol-5-yl)-N-(2,3-dihydro-1-benzofuran-5-ylmethyl)- |
| | N-[3-(trifluoromethyl)benzyl]methanamine |
| 2319. | 1-(1-butyl-2,4-diphenyl-1H-imidazol-5-yl)-N-[4-(difluoromethoxy)benzyl]-N-(2,3- |
| | dihydro-1-benzofuran-5-ylmethyl)methanamine |
| 2320. | 1-(1-butyl-2,4-diphenyl-1H-imidazol-5-yl)-N-(4-methoxy-3,5-dimethylbenzyl)-N-[3- |
| | (trifluoromethyl)benzyl]methanamine |
| 2321. | 1-(1-butyl-2,4-diphenyl-1H-imidazol-5-yl)-N-[4-(difluoromethoxy)benzyl]-N-(4- |
| | methoxy-3,5-dimethylbenzyl)methanamine |
| 2322. | 1-(1,3-benzodioxol-5-yl)-N-(1,3-benzodioxol-5-ylmethyl)-N-{[1-butyl-2-phenyl-4- |
| | (trifluoromethyl)-1H-imidazol-5-yl]methyl}methanamine |
| 2323. | 1-butyl-N-[4-(difluoromethoxy)benzyl]-N-[4-(difluoromethoxy)-3-fluorobenzyl]-5,5- |
| | dimethyl-2-phenyl-4,5,6,7-tetrahydro-1H-benzimidazol-7-amine |
| 2324. | N-{[1-butyl-2-phenyl-4-(trifluoromethyl)-1H-imidazol-5-yl]methyl}-N-[4- |
| | (difluoromethoxy)benzyl]ethanamine |
| 2325. | methyl 4-({butyl[(1-butyl-2,4-diphenyl-1H-imidazol-5-yl)methyl]amino}methyl)-2- |
| | methoxybenzoate |
| 2326. | methyl 4-{[[(1-butyl-2,4-diphenyl-1H-imidazol-5- |
| | yl)methyl](cyclohexylmethyl)amino]methyl}-2-methoxybenzoate |
| 2327. | methyl 4-({(1,3-benzodioxol-5-ylmethyl)[(1-butyl-2,4-diphenyl-1H-imidazol-5- |
| | yl)methyl]amino}methyl)-2-methoxybenzoate |
| 2328. | methyl 4-{[[(1-butyl-2,4-diphenyl-1H-imidazol-5-yl)methyl](isopentyl)amino]methyl}- |
| | 2-methoxybenzoate |
| 2329. | 4-({butyl[(1-butyl-2,4-diphenyl-1H-imidazol-5-yl)methyl]amino}methyl)-2- |
| | methoxybenzamide |
| 2330. | 4-({butyl[(1-butyl-2,4-diphenyl-1H-imidazol-5-yl)methyl]amino}methyl)-2- |
| | hydroxybenzamide |
| 2331. | 4-({(1,3-benzodioxol-5-ylmethyl)[(1-butyl-2,4-diphenyl-1H-imidazol-5- |
| | yl)methyl]amino}methyl)-2-methoxybenzamide |
| 2332. | 1-(1,3-benzodioxol-5-yl)-N-(1,3-benzodioxol-5-ylmethyl)-N-({1-butyl-4-[4- |
| | (ethylthio)phenyl]-2-phenyl-1H-imidazol-5-yl}methyl)methanamine |
| 2333. | 5-({{[1-butyl-2-phenyl-4-(trifluoromethyl)-1H-imidazol-5-yl]methyl}[4- |
| | (difluoromethoxy)benzyl]amino}methyl)-1,3-benzoxazol-2(3H)-one |
| 2334. | 1-(1,3-benzodioxol-5-yl)-N-(1,3-benzodioxol-5-ylmethyl)-N-({1-butyl-4-[2- |
| | (methylthio)phenyl]-2-phenyl-1H-imidazol-5-yl}methyl)methanamine |
| 2335. | 1-(1,3-benzodioxol-5-yl)-N-(1,3-benzodioxol-5-ylmethyl)-N-({1-butyl-4-[(E)-2-(4- |
| | fluorophenyl)ethenyl]-2-phenyl-1H-imidazol-5-yl}methyl)methanamine |
| 2336. | 1-(1,3-benzodioxol-5-yl)-N-(1,3-benzodioxol-5-ylmethyl)-N-({1-butyl-4-[3- |
| | (methylthio)phenyl]-2-phenyl-1H-imidazol-5-yl}methyl)methanamine |
| 2337. | 1-(1,3-benzodioxol-5-yl)-N-{[4-(1,3-benzodioxol-5-yl)-1-butyl-2-phenyl-1H-imidazol- |
| | 5-yl]methyl}-N-(1,3-benzodioxol-5-ylmethyl)methanamine |
| 2338. | 1-(1,3-benzodioxol-5-yl)-N-(1,3-benzodioxol-5-ylmethyl)-N-{[1-butyl-4- |
| | (dimethoxymethyl)-2-phenyl-1H-imidazol-5-yl]methyl}methanamine |
| 2339. | 1-(1,3-benzodioxol-5-yl)-N-(1,3-benzodioxol-5-ylmethyl)-N-{[1-butyl-4- |
| | (difluoromethyl)-2-phenyl-1H-imidazol-5-yl]methyl}methanamine |
| 2340. | N,N-bis(1,3-benzodioxol-5-ylmethyl)-N-({1-butyl-4-[(methylamino)methyl]-2-phenyl- |
| | 1H-imidazol-5-yl}methyl)amine |
| 2341. | (5-{[bis(1,3-benzodioxol-5-ylmethyl)amino]methyl}-1-butyl-2-phenyl-1H-imidazol-4- |
| | yl)methanol |
| 2342. | 1-(1,3-benzodioxol-5-yl)-N-(1,3-benzodioxol-5-ylmethyl)-N-{[1-butyl-4-(3- |
| | methoxyphenyl)-2-phenyl-1H-imidazol-5-yl]methyl}methanamine |
| 2343. | 1-(1,3-benzodioxol-5-yl)-N-(1,3-benzodioxol-5-ylmethyl)-N-({1-butyl-2-phenyl-4-[4- |
| | (trifluoromethoxy)phenyl]-1H-imidazol-5-yl}methyl)methanamine |
| 2344. | 1-(1,3-benzodioxol-5-yl)-N-(1,3-benzodioxol-5-ylmethyl)-N-{[1-butyl-4-(3,4- |
| | difluorophenyl)-2-phenyl-1H-imidazol-5-yl]methyl}methanamine |
| 2345. | 1-(1,3-benzodioxol-5-yl)-N-{[1-butyl-2-phenyl-4-(trifluoromethyl)-1H-imidazol-5- |
| | yl]methyl}-N-(3-ethoxybenzyl)methanamine |
| 2346. | 1-(1,3-benzodioxol-5-yl)-N-{[1-butyl-2-phenyl-4-(trifluoromethyl)-1H-imidazol-5- |
| | yl]methyl}-N-[3-(1,1,2,2-tetrafluoroethoxy)benzyl]methanamine |
| 2347. | 1-(1,3-benzodioxol-5-yl)-N-(1,3-benzodioxol-5-ylmethyl)-N-{[1-butyl-4-(3- |
| | nitrophenyl)-2-phenyl-1H-imidazol-5-yl]methyl}methanamine |
| 2348. | 1-(1-butyl-2,4-diphenyl-1H-imidazol-5-yl)-N-(2,6-difluorobenzyl)-N-(4- |
| | methoxybenzyl)methanamine |
| 2349. | 4-({butyl[(1-butyl-2,4-diphenyl-1H-imidazol-5-yl)methyl]amino}methyl)-2- |
| | hydroxybenzoic acid |
| 2350. | 4-{[[(1-butyl-2,4-diphenyl-1H-imidazol-5- |
| | yl)methyl](cyclohexylmethyl)amino]methyl}-2-methoxybenzoic acid |
| 2351. | 4-{[[(1-butyl-2,4-diphenyl-1H-imidazol-5- |
| | yl)methyl](cyclohexylmethyl)amino]methyl}-2-hydroxybenzamide |
| 2352. | 4-({benzyl[(1-butyl-2,4-diphenyl-1H-imidazol-5-yl)methyl]amino}methyl)-2- |
| | hydroxybenzamide |
| 2353. | 4-{[[(1-butyl-2,4-diphenyl-1H-imidazol-5-yl)methyl](isopentyl)amino]methyl}-2- |
| | methoxybenzamide |
| 2354. | 4-({benzyl[(1-butyl-2,4-diphenyl-1H-imidazol-5-yl)methyl]amino}methyl)-2- |
| | methoxybenzoic acid |
| 2355. | 4-{[[(1-butyl-2,4-diphenyl-1H-imidazol-5-yl)methyl](isopentyl)amino]methyl}-2- |
| | hydroxybenzamide |
| 2356. | 4-({benzyl[(1-butyl-2,4-diphenyl-1H-imidazol-5-yl)methyl]amino}methyl)-2- |
| | hydroxybenzoic acid |
| 2357. | 1-(1-butyl-2,4-diphenyl-1H-imidazol-5-yl)-N-(2-chloro-6-fluorobenzyl)-N-(4- |
| | methoxybenzyl)methanamine |
| 2358. | 1-(1,3-benzodioxol-5-yl)-N-(1,3-benzodioxol-5-ylmethyl)-N-{[1-butyl-4-(4- |
| | methylphenyl)-2-phenyl-1H-imidazol-5-yl]methyl}methanamine |
| 2359. | 1-[1-butyl-2-(2-methoxyphenyl)-4-(trifluoromethyl)-1H-imidazol-5-yl]-N,N-bis(3- |
| | ethoxybenzyl)methanamine |
| 2360. | 1-[1-butyl-2-phenyl-4-(trifluoromethyl)-1H-imidazol-5-yl]-N,N-bis(3- |
| | ethoxybenzyl)methanamine |
| 2361. | 4-(5-{[bis(1,3-benzodioxol-5-ylmethyl)amino]methyl}-1-butyl-2-phenyl-1H-imidazol- |
| | 4-yl)benzonitrile |
| 2362. | 1-(1,3-benzodioxol-5-yl)-N-(1,3-benzodioxol-5-ylmethyl)-N-({1-butyl-2-phenyl-4-[4- |
| | (trifluoromethyl)phenyl]-1H-imidazol-5-yl}methyl)methanamine |
| 2363. | 7-[(butyl{[1-butyl-4-(3-methoxyphenyl)-2-phenyl-1H-imidazol-5- |
| | yl]methyl}amino)methyl]quinazolin-4-amine |
| 2364. | 1-(1,3-benzodioxol-5-yl)-N-[(1-butyl-4-chloro-2-phenyl-1H-imidazol-5-yl)methyl]-N- |
| | (3-ethoxybenzyl)methanamine |
| 2365. | methyl 4-[(butyl{[1-butyl-2-phenyl-4-(trifluoromethyl)-1H-imidazol-5- |
| | yl]methyl}amino)methyl]-2-methoxybenzoate |
| 2366. | 4-[(butyl{[1-butyl-2-phenyl-4-(trifluoromethyl)-1H-imidazol-5- |
| | yl]methyl}amino)methyl]-2-methoxybenzoic acid |
| 2367. | 4-[(butyl{[1-butyl-2-phenyl-4-(trifluoromethyl)-1H-imidazol-5- |
| | yl]methyl}amino)methyl]-2-methoxybenzamide |
| 2368. | 4-[(butyl{[1-butyl-2-phenyl-4-(trifluoromethyl)-1H-imidazol-5- |
| | yl]methyl}amino)methyl]-2-hydroxybenzamide |
| 2369. | 1-(1,3-benzodioxol-5-yl)-N-[(1-butyl-2,4-diphenyl-1H-imidazol-5-yl)methyl]-N-(1H- |
| | indol-5-ylmethyl)methanamine |
| 2370. | N-benzyl-1-(1-butyl-2,4-diphenyl-1H-imidazol-5-yl)-N-(1H-indol-5- |
| | ylmethyl)methanamine |
| 2371. | N-[(1-butyl-2,4-diphenyl-1H-imidazol-5-yl)methyl]-N-(1H-indol-5-ylmethyl)butan-1- |
| | amine |
| 2372. | 1-(1,3-benzodioxol-5-yl)-N-[(1-butyl-2-phenyl-1H-imidazol-5-yl)methyl]-N-[4- |
| | (difluoromethoxy)benzyl]methanamine |
| 2373. | 5-{[bis(1,3-benzodioxol-5-ylmethyl)amino]methyl}-1-butyl-2-phenyl-1H-imidazole-4- |
| | carboxylic acid |
| 2374. | methyl 5-{[bis(1,3-benzodioxol-5-ylmethyl)amino]methyl}-1-butyl-2-phenyl-1H- |
| | imidazole-4-carboxylate |
| 2375. | 1-(1-butyl-2-phenyl-1H-imidazol-5-yl)-N,N-bis(3-ethoxybenzyl)methanamine |
| 2376. | 1-(1-butyl-2-phenyl-1H-imidazol-5-yl)-N-[3-difluoromethoxy)benzyl]-N-[4- |
| | (difluoromethoxy)benzyl]methanamine |
| 2377. | 2-(5-{[bis(1,3-benzodioxol-5-ylmethyl)amino]methyl}-1-butyl-2-phenyl-1H-imidazol- |
| | 4-yl)propan-2-ol |
| 2378. | 1-(1,3-benzodioxol-5-yl)-N-[(4-bromo-1-butyl-2-phenyl-1H-imidazol-5-yl)methyl]-N- |
| | [4-(difluoromethoxy)benzyl]methanamine |
| 2379. | 1-[1-butyl-4-(4-methoxyphenyl)-2-phenyl-1H-imidazol-5-yl]-N,N-bis(3- |
| | ethoxybenzyl)methanamine |
| 2380. | 1-[1-butyl-4-(3-methoxyphenyl)-2-phenyl-1H-imidazol-5-yl]-N,N-bis(3- |
| | ethoxybenzyl)methanamine |
| 2381. | 1-(1,3-benzodioxol-5-yl)-N-(1,3-benzodioxol-5-ylmethyl)-N-[(1-butyl-4-ethoxy-2- |
| | phenyl-1H-imidazol-5-yl)methyl]methanamine |
| 2382. | 1-[1-butyl-2-(3-methoxyphenyl)-4-phenyl-1H-imidazol-5-yl]-N,N-bis(3- |
| | ethoxybenzyl)methanamine |
| 2383. | N-[(4-bromo-1-butyl-2-phenyl-1H-imidazol-5-yl)methyl]-N,N-bis(3- |
| | ethoxybenzyl)amine |
| 2384. | N-[(4-bromo-1-butyl-2-phenyl-1H-imidazol-5-yl)methyl]-N,N-bis[4- |
| | (difluoromethoxy)benzyl]amine |
| 2385. | N-[(1-butyl-4-chloro-2-phenyl-1H-imidazol-5-yl)methyl]-N,N-bis(3- |
| | ethoxybenzyl)amine |
| 2386. | N-[(1-butyl-4-chloro-2-phenyl-1H-imidazol-5-yl)methyl]-N,N-bis[4- |
| | (difluoromethoxy)benzyl]amine |
| 2387. | 1-[1-butyl-4-(4-methoxyphenyl)-2-phenyl-1H-imidazol-5-yl]-N-[4- |
| | (difluoromethoxy)benzyl]-N-(3-ethoxybenzyl)methanamine |
| 2388. | 1-(1,3-benzodioxol-5-yl)-N-{[1-butyl-4-(4-methoxyphenyl)-2-phenyl-1H-imidazol-5- |
| | yl]methyl}-N-[4-(difluoromethoxy)benzyl]methanamine |
| 2389. | 1-[1-butyl-4-(4-methoxyphenyl)-2-phenyl-1H-imidazol-5-yl]-N,N-bis[4- |
| | (difluoromethoxy)benzyl]methanamine |
| 2390. | 1-[1-butyl-4-(3-methoxyphenyl)-2-phenyl-1H-imidazol-5-yl]-N-[4- |
| | (difluoromethoxy)benzyl]-N-(3-ethoxybenzyl)methanamine |
| 2391. | 1-(1,3-benzodioxol-5-yl)-N-{[1-butyl-4-(3-methoxyphenyl)-2-phenyl-1H-imidazol-5- |
| | yl]methyl}-N-[4-(difluoromethoxy)benzyl]methanamine |
| 2392. | 1-[1-butyl-4-(3-methoxyphenyl)-2-phenyl-1H-imidazol-5-yl]-N,N-bis[4- |
| | (difluoromethoxy)benzyl]methanamine |
| 2393. | N-{[1-butyl-4-(3-ethoxyphenyl)-2-phenyl-1H-imidazol-5-yl]methyl}-N-[4- |
| | (difluoromethoxy)benzyl]-N-(3-ethoxybenzyl)amine |
| 2394. | 1-(1,3-benzodioxol-5-yl)-N-{[1-butyl-4-(3-ethoxyphenyl)-2-phenyl-1H-imidazol-5- |
| | yl]methyl}-N-[4-(difluoromethoxy)benzyl]methanamine |
| 2395. | N-{[1-butyl-4-(3-ethoxyphenyl)-2-phenyl-1H-imidazol-5-yl]methyl}-N,N-bis(3- |
| | ethoxybenzyl)amine |
| 2396. | N-{[1-butyl-4-(3-ethoxyphenyl)-2-phenyl-1H-imidazol-5-yl]methyl}-N,N-bis[4- |
| | (difluoromethoxy)benzyl]amine |
| 2397. | 1-(1-butyl-2-phenyl-4-thien-3-yl-1H-imidazol-5-yl)-N-[4-(difluoromethoxy)benzyl]-N- |
| | (3-ethoxybenzyl)methanamine |
| 2398. | 1-(1,3-benzodioxol-5-yl)-N-[(1-butyl-2-phenyl-4-thien-3-yl-1H-imidazol-5-yl)methyl]- |
| | N-[4-(difluoromethoxy)benzyl]methanamine |
| 2399. | 4-{[[(1-butyl-2,4-diphenyl-1H-imidazol-5-yl)methyl](4-hydroxy-3,5- |
| | dimethylbenzyl)amino]methyl}benzenesulfonamide |
| 2400. | 4-{[[(1-butyl-2,4-diphenyl-1H-imidazol-5-yl)methyl](4-hydroxy-3- |
| | methylbenzyl)amino]methyl}benzenesulfonamide |
| 2401. | 4-({[(1-butyl-2,4-diphenyl-1H-imidazol-5-yl)methyl][4-(difluoromethoxy)-3- |
| | methylbenzyl]amino}methyl)benzenesulfonamide |
| 2402. | N-benzyl-1-[3,4-bis(difluoromethoxy)phenyl]-N-[(1-butyl-2,4-diphenyl-1H-imidazol-5- |
| | yl)methyl]methanamine |
| 2403. | N-[(1-butyl-2,4-diphenyl-1H-imidazol-5-yl)methyl]-N-[4- |
| | (difluoromethoxy)benzyl]propan-1-amine |
| 2404. | N-[(1-butyl-2,4-diphenyl-1H-imidazol-5-yl)methyl]-N-[4- |
| | (difluoromethoxy)benzyl]pentan-1-amine |
| 2405. | N-[(1-butyl-2,4-diphenyl-1H-imidazol-5-yl)methyl]-N-[4- |
| | (difluoromethoxy)benzyl]cyclopentanamine |
| 2406. | N-[(1-butyl-2,4-diphenyl-1H-imidazol-5-yl)methyl]-N-[4- |
| | (difluoromethoxy)benzyl]propan-2-amine |
| 2407. | N-[(1-butyl-2,4-diphenyl-1H-imidazol-5-yl)methyl]-N-[4-(difluoromethoxy)benzyl]- |
| | 2,2-dimethylpropan-1-amine |
| 2408. | N-[(1-butyl-2,4-diphenyl-1H-imidazol-5-yl)methyl]-N-[4-(difluoromethoxy)benzyl]-3- |
| | methylbutan-1-amine |
| 2409. | N-[(1-butyl-2,4-diphenyl-1H-imidazol-5-yl)methyl]-N-[4- |
| | (difluoromethoxy)benzyl]cyclohexanamine |
| 2410. | N-[(1-butyl-2,4-diphenyl-1H-imidazol-5-yl)methyl]-N-[4- |
| | (difluoromethoxy)benzyl]ethanamine |
| 2411. | N-{[1-butyl-2-phenyl-4-(trifluoromethyl)-1H-imidazol-5-yl]methyl}-N-[4- |
| | (difluoromethoxy)benzyl]pentan-1-amine |
| 2412. | N-benzyl-1-[1-butyl-2-phenyl-4-(trifluoromethyl)-1H-imidazol-5-yl]-N-[4- |
| | (difluoromethoxy)benzyl]methanamine |
| 2413. | N,1-dibutyl-N-[4-(difluoromethoxy)benzyl]-2-phenyl-4,5,6,7-tetrahydro-1H- |
| | benzimidazol-7-amine |
| 2414. | N,1-dibutyl-N-(4-methoxy-3,5-dimethylbenzyl)-2-phenyl-4,5,6,7-tetrahydro-1H- |
| | benzimidazol-7-amine |
| 2415. | 4-{[butyl(1-butyl-2-phenyl-4,5,6,7-tetrahydro-1H-benzimidazol-7-yl)amino]methyl}- |
| | 2,6-dimethylphenol |
| 2416. | N-{[1-butyl-2-phenyl-4-(trifluoromethyl)-1H-imidazol-5-yl]methyl}-N-[4- |
| | (difluoromethoxy)benzyl]butan-1-amine |
| 2417. | 4-[(butyl{[1-butyl-2-phenyl-4-(trifluoromethyl)-1H-imidazol-5- |
| | yl]methyl}amino)methyl]benzenesulfonamide |
| 2418. | 1-[1-butyl-2-phenyl-4-(trifluoromethyl)-1H-imidazol-5-yl]-N-[4- |
| | (difluoromethoxy)benzyl]-N-(4-methoxy-3,5-dimethylbenzyl)methanamine |
| 2419. | 1-[1-butyl-2-phenyl-4-(trifluoromethyl)-1H-imidazol-5-yl]-N-[4- |
| | (difluoromethoxy)benzyl]-N-[4-(difluoromethoxy)-3,5-dimethylbenzyl]methanamine |
| 2420. | 1-(1,3-benzodioxol-5-yl)-N-{[1-butyl-2-phenyl-4-(trifluoromethyl)-1H-imidazol-5- |
| | yl]methyl}-N-[4-(difluoromethoxy)benzyl]methanamine |
| 2421. | 1-[1-butyl-2-phenyl-4-(trifluoromethyl)-1H-imidazol-5-yl]-N-[4- |
| | (difluoromethoxy)benzyl]-N-[4-(difluoromethoxy)-3-fluorobenzyl]methanamine |
| 2422. | N-{[1-butyl-2-phenyl-4-(trifluoromethyl)-1H-imidazol-5-yl]methyl}-N-(4-methoxy- |
| | 3,5-dimethylbenzyl)butan-1-amine |
| 2423. | 4-({{[1-butyl-2-phenyl-4-(trifluoromethyl)-1H-imidazol-5-yl]methyl}[4- |
| | (difluoromethoxy)benzyl]amino}methyl)benzenesulfonamide |
| 2424. | 1-[1-butyl-2-phenyl-4-(trifluoromethyl)-1H-imidazol-5-yl]-N,N-bis[4- |
| | (difluoromethoxy)benzyl]methanamine |
| 2425. | 5-({benzyl[(1-butyl-2,4-diphenyl-1H-imidazol-5-yl)methyl]amino}methyl)-2- |
| | (difluoromethoxy)phenol |
| 2426. | 5-({butyl[(1-butyl-2,4-diphenyl-1H-imidazol-5-yl)methyl]amino}methyl)-2- |
| | (difluoromethoxy)phenol |
| 2427. | 4-{[[(1-butyl-2,4-diphenyl-1H-imidazol-5-yl)methyl](4-methoxy-3,5- |
| | dimethylbenzyl)amino]methyl}benzenesulfonamide |
| 2428. | 4-{[[(1-butyl-2,4-diphenyl-1H-imidazol-5-yl)methyl](2-chloro-4- |
| | hydroxybenzyl)amino]methyl}benzenesulfonamide |
| 2429. | 4-{[[(1-butyl-2,4-diphenyl-1H-imidazol-5-yl)methyl](3-fluoro-4- |
| | methoxybenzyl)amino]methyl}benzenesulfonamide |
| 2430. | 4-({[4-(aminosulfonyl)benzyl][(1-butyl-2,4-diphenyl-1H-imidazol-5- |
| | yl)methyl]amino}methyl)phenyl acetate |
| 2431. | 4-{[[(1-butyl-2,4-diphenyl-1H-imidazol-5-yl)methyl](4- |
| | methoxybenzyl)amino]methyl}phenyl acetate |
| 2432. | 2-(5-{[bis(1,3-benzodioxol-5-ylmethyl)amino]methyl}-1-butyl-2-phenyl-1H-imidazol- |
| | 4-yl)phenol |
| 2433. | 4-({butyl[(1-butyl-2,4-diphenyl-1H-imidazol-5-yl)methyl]amino}methyl)-2- |
| | methylphenyl 3-methylbutanoate |
| 2434. | 4-({benzyl[(1-butyl-2,4-diphenyl-1H-imidazol-5-yl)methyl]amino}methyl)-2- |
| | methylphenyl 3-methylbutanoate |
| 2435. | 2-(5-{[bis(1,3-benzodioxol-5-ylmethyl)amino]methyl}-1-butyl-4-phenyl-1H-imidazol- |
| | 2-yl)phenol |
| 2436. | 4-(5-{[bis(1,3-benzodioxol-5-ylmethyl)amino]methyl}-1-butyl-2-phenyl-1H-imidazol- |
| | 4-yl)phenol |
| 2437. | N-(1,3-benzodioxol-5-ylmethyl)-N-{[1-butyl-2-phenyl-4-(trifluoromethyl)-1H- |
| | imidazol-5-yl]methyl}butan-1-amine |
| 2438. | N-{[1-butyl-2-phenyl-4-(trifluoromethyl)-1H-imidazol-5-yl]methyl}-N-[4- |
| | (difluoromethoxy)-3-fluorobenzyl]butan-1-amine |
| 2439. | N-{[1-butyl-2-phenyl-4-(trifluoromethyl)-1H-imidazol-5-yl]methyl}-N-[4- |
| | (difluoromethoxy)-3,5-dimethylbenzyl]butan-1-amine |
| 2440. | 4-[(benzyl{[1-butyl-2-phenyl-4-(trifluoromethyl)-1H-imidazol-5- |
| | yl]methyl}amino)methyl]benzenesulfonamide |
| 2441. | 1-(1-butyl-2,4-diphenyl-1H-imidazol-5-yl)-N-(4-methoxybenzyl)-N-(4-methoxy-3,5- |
| | dimethylbenzyl)methanamine |
| 2442. | 1-(1-butyl-2,4-diphenyl-1H-imidazol-5-yl)-N-(cyclohexylmethyl)-N-(4-methoxy-3,5- |
| | dimethylbenzyl)methanamine |
| 2443. | 4-({benzyl[(1-butyl-2,4-diphenyl-1H-imidazol-5-yl)methyl]amino}methyl)-2- |
| | fluorophenol |
| 2444. | 4-({butyl[(1-butyl-2,4-diphenyl-1H-imidazol-5-yl)methyl]amino}methyl)-2- |
| | fluorophenol |
| 2445. | 4-{[[(1-butyl-2,4-diphenyl-1H-imidazol-5-yl)methyl](3-fluoro-4-hydroxybenzyl)amino] |
| | methyl}benzenesulfonamide |
| 2446. | 4-{[[(1-butyl-2,4-diphenyl-1H-imidazol-5-yl)methyl](4- |
| | methoxybenzyl)amino]methyl}-2-fluorophenol |
| 2447. | 1-(1-butyl-2,4-diphenyl-1H-imidazol-5-yl)-N-(cyclohex-3-en-1-ylmethyl)-N- |
| | (cyclohexylmethyl)methanamine |
| 2448. | 4-{[[(1-butyl-2,4-diphenyl-1H-imidazol-5- |
| | yl)methyl](cyclohexylmethyl)amino]methyl}cyclohexane-1,2-diol |
| 2449. | 1-(1-butyl-2,4-diphenyl-1H-imidazol-5-yl)-N-(cyclohexylmethyl)-N-(hexahydro-1,3- |
| | benzodioxol-5-ylmethyl)methanamine |
| 2450. | 1-(1-butyl-2,4-diphenyl-1H-imidazol-5-yl)-N-(cyclohex-3-en-1-ylmethyl)-N-[4- |
| | (difluoromethoxy)benzyl]methanamine |
| 2451. | 4-({[(1-butyl-2,4-diphenyl-1H-imidazol-5-yl)methyl][4- |
| | (difluoromethoxy)benzyl]amino}methyl)cyclohexane-1,2-diol |
| 2452. | 1-(1-butyl-2,4-diphenyl-1H-imidazol-5-yl)-N-[4-(difluoromethoxy)benzyl]-N- |
| | (hexahydro-1,3-benzodioxol-5-ylmethyl)methanamine |
| 2453. | 1-(1-butyl-2,4-diphenyl-1H-imidazol-5-yl)-N-[(2,2-difluoro-1,3-benzodioxol-5- |
| | yl)methyl]-N-[4-(difluoromethoxy)benzyl]methanamine |
| 2454. | 1-(1-butyl-2,4-diphenyl-1H-imidazol-5-yl)-N-(cyclohexylmethyl)-N-[(2,2-difluoro-1,3- |
| | benzodioxol-5-yl)methyl]methanamine |
| 2455. | N-[(1-butyl-2,4-diphenyl-1H-imidazol-5-yl)methyl]-N-[(2,2-difluoro-1,3-benzodioxol- |
| | 5-yl)methyl]-3-methylbutan-1-amine |
| 2456. | N-[(1-butyl-2,4-diphenyl-1H-imidazol-5-yl)methyl]-N-[4- |
| | (difluoromethoxy)benzyl]hexan-1-amine |
| 2457. | N-[(1-butyl-2,4-diphenyl-1H-imidazol-5-yl)methyl]-N-[(2,2-difluoro-1,3-benzodioxol- |
| | 5-yl)methyl]butan-1-amine |
| 2458. | N-[(1-butyl-2,4-diphenyl-1H-imidazol-5-yl)methyl]-N-[4- |
| | (difluoromethoxy)benzyl]heptan-1-amine |
| 2459. | N-[(1-butyl-2,4-diphenyl-1H-imidazol-5-yl)methyl]-N-[4- |
| | (difluoromethoxy)benzyl]octan-1-amine |
| 2460. | N-[(1-butyl-2,4-diphenyl-1H-imidazol-5-yl)methyl]-N-[4-(difluoromethoxy)benzyl]- |
| | 3,3-dimethylbutan-1-amine |
| 2461. | N-[(1-butyl-2,4-diphenyl-1H-imidazol-5-yl)methyl]-2-cyclohex-1-en-1-yl-N-[4- |
| | (difluoromethoxy)benzyl]ethanamine |
| 2462. | 4-{[[(1-butyl-2,4-diphenyl-1H-imidazol-5- |
| | yl)methyl](cyclohexylmethyl)amino]methyl}-2-fluorophenol |
| 2463. | N-benzyl-1-(1-butyl-2,4-diphenyl-1H-imidazol-5-yl)-N-[(2,2-difluoro-1,3-benzodioxol- |
| | 5-yl)methyl]methanamine |
| 2464. | 4-({[(1-butyl-2,4-diphenyl-1H-imidazol-5-yl)methyl][(2,2-difluoro-1,3-benzodioxol-5- |
| | yl)methyl]amino}methyl)benzenesulfonamide |
| 2465. | 1-(1-butyl-2,4-diphenyl-1H-imidazol-5-yl)-N-[(2,2-difluoro-1,3-benzodioxol-5- |
| | yl)methyl]-N-(4-methoxybenzyl)methanamine |
| 2466. | 4-({[(1-butyl-2,4-diphenyl-1H-imidazol-5-yl)methyl][3-(1,1,2,2- |
| | tetrafluoroethoxy)benzyl]amino}methyl)benzenesulfonamide |
| 2467. | 4-{[[(1-butyl-2,4-diphenyl-1H-imidazol-5-yl)methyl](3- |
| | ethoxybenzyl)amino]methyl}benzenesulfonamide |
| 2468. | 1-(1-butyl-2,4-diphenyl-1H-imidazol-5-yl)-N-(4-methoxybenzyl)-N-[3-(1,1,2,2- |
| | tetrafluoroethoxy)benzyl]methanamine |
| 2469. | 1-(1-butyl-2,4-diphenyl-1H-imidazol-5-yl)-N-(3-ethoxybenzyl)-N-(4- |
| | methoxybenzyl)methanamine |
| 2470. | N-benzyl-1-[1-butyl-2-phenyl-4-(trifluoromethyl)-1H-imidazol-5-yl]-N-[4- |
| | (difluoromethoxy)-3-fluorobenzyl]methanamine |
| 2471. | N-benzyl-1-[1-butyl-2-phenyl-4-(trifluoromethyl)-1H-imidazol-5-yl]-N-(4-methoxy- |
| | 3,5-dimethylbenzyl)methanamine |
| 2472. | N,N-dibenzyl-1-[1-butyl-2-phenyl-4-(trifluoromethyl)-1H-imidazol-5-yl]methanamine |
| 2473. | 1-(1,3-benzodioxol-5-yl)-N-benzyl-N-{[1-butyl-2-phenyl-4-(trifluoromethyl)-1H- |
| | imidazol-5-yl]methyl}methanamine |
| 2474. | N-benzyl-1-[1-butyl-2-phenyl-4-(trifluoromethyl)-1H-imidazol-5-yl]-N-[3-(1,1,2,2- |
| | tetrafluoroethoxy)benzyl]methanamine |
| 2475. | 4-{[{[1-butyl-2-phenyl-4-(trifluoromethyl)-1H-imidazol-5-yl]methyl}(4-hydroxy-3,5- |
| | dimethylbenzyl)amino]methyl}benzenesulfonamide |
| 2476. | 4-[(benzyl{[1-butyl-2-phenyl-4-(trifluoromethyl)-1H-imidazol-5- |
| | yl]methyl}amino)methyl]-2-fluorophenol |
| 2477. | 1-[1-butyl-2-phenyl-4-(trifluoromethyl)-1H-imidazol-5-yl]-N-[4- |
| | (difluoromethoxy)benzyl]-N-[3-(1,1,2,2-tetrafluoroethoxy)benzyl]methanamine |
| 2478. | 1-[1-butyl-2-phenyl-4-(trifluoromethyl)-1H-imidazol-5-yl]-N-[4- |
| | (difluoromethoxy)benzyl]-N-(3-fluoro-4-methoxybenzyl)methanamine |
| 2479. | 4-({{[1-butyl-2-phenyl-4-(trifluoromethyl)-1H-imidazol-5-yl]methyl}[4- |
| | (difluoromethoxy)benzyl]amino}methyl)-2-fluorophenol |
| 2480. | 4-({{[1-butyl-2-phenyl-4-(trifluoromethyl)-1H-imidazol-5-yl]methyl}[4- |
| | (difluoromethoxy)-3-fluorobenzyl]amino}methyl)benzenesulfonamide |
| 2481. | 4-{[{[1-butyl-2-phenyl-4-(trifluoromethyl)-1H-imidazol-5-yl]methyl}(2-chloro-4- |
| | hydroxybenzyl)amino]methyl}benzenesulfonamide |
| 2482. | 4-[((1,3-benzodioxol-5-ylmethyl){[1-butyl-2-phenyl-4-(trifluoromethyl)-1H-imidazol- |
| | 5-yl]methyl}amino)methyl]benzenesulfonamide |
| 2483. | 1-(1,3-benzodioxol-5-yl)-N-(1,3-benzodioxol-5-ylmethyl)-N-[(1-butyl-4-chloro-2- |
| | phenyl-1H-imidazol-5-yl)methyl]methanamine |
| 2484. | N-benzyl-1-[1-butyl-2-phenyl-4-(trifluoromethyl)-1H-imidazol-5-yl]-N-(3-fluoro-4- |
| | methoxybenzyl)methanamine |
| 2485. | 4-(5-{[bis(1,3-benzodioxol-5-ylmethyl)amino]methyl}-1-butyl-4-phenyl-1H-imidazol- |
| | 2-yl)phenol |
| 2486. | 4-({[(1-butyl-2,4-diphenyl-1H-imidazol-5-yl)methyl][3- |
| | (difluoromethoxy)benzyl]amino}methyl)benzenesulfonamide |
| 2487. | 4-{[[(1-butyl-2,4-diphenyl-1H-imidazol-5-yl)methyl](3- |
| | methoxybenzyl)amino]methyl}benzenesulfonamide |
| 2488. | 1-(1-butyl-2,4-diphenyl-1H-imidazol-5-yl)-N-[3-(difluoromethoxy)benzyl]-N-(4- |
| | methoxybenzyl)methanamine |
| 2489. | 1-(1-butyl-2,4-diphenyl-1H-imidazol-5-yl)-N-(3-methoxybenzyl)-N-(4- |
| | methoxybenzyl)methanamine |
| 2490. | 1-[1-butyl-2-phenyl-4-(trifluoromethyl)-1H-imidazol-5-yl]-N-[(2,2-difluoro-1,3- |
| | benzodioxol-5-yl)methyl]-N-[4-(difluoromethoxy)benzyl]methanamine |
| 2491. | 4-({{[1-butyl-2-phenyl-4-(trifluoromethyl)-1H-imidazol-5-yl]methyl}[(2,2-difluoro- |
| | 1,3-benzodioxol-5-yl)methyl]amino}methyl)benzenesulfonamide |
| 2492. | 1-(1,3-benzodioxol-5-yl)-N-(1,3-benzodioxol-5-ylmethyl)-N-[(4-bromo-1-butyl-2- |
| | phenyl-1H-imidazol-5-yl)methyl]methanamine |
| 2493. | 1-(1,3-benzodioxol-5-yl)-N-(1,3-benzodioxol-5-ylmethyl)-N-{[1-butyl-2-(2- |
| | methoxyphenyl)-4-(trifluoromethyl)-1H-imidazol-5-yl]methyl}methanamine |
| 2494. | 1-[1-butyl-2-phenyl-4-(trifluoromethyl)-1H-imidazol-5-yl]-N-[4- |
| | (difluoromethoxy)benzyl]-N-(3-ethoxybenzyl)methanamine |
| 2495. | N-benzyl-1-[1-butyl-2-phenyl-4-(trifluoromethyl)-1H-imidazol-5-yl]-N-(3- |
| | ethoxybenzyl)methanamine |
| 2496. | 4-({(1,3-benzodioxol-5-ylmethyl)[(1R)-1-(1-butyl-2,4-diphenyl-1H-imidazol-5- |
| | yl)ethyl]amino}methyl)benzenesulfonamide |
| 2497. | 4-({(1,3-benzodioxol-5-ylmethyl)[(1S)-1-(1-butyl-2,4-diphenyl-1H-imidazol-5- |
| | yl)ethyl]amino}methyl)benzenesulfonamide |
| 2498. | 4-{[[(1R)-1-(1-butyl-2,4-diphenyl-1H-imidazol-5- |
| | yl)ethyl](cyclohexylmethyl)amino]methyl}benzenesulfonamide |
| 2499. | 4-({(1,3-benzodioxol-5-ylmethyl)[(1S)-1-(1-butyl-2,4-diphenyl-1H-imidazol-5- |
| | yl)pentyl]amino}methyl)benzenesulfonamide |
| 2500. | N-(1,3-benzodioxol-5-ylmethyl)-1-(1-butyl-2-phenyl-1H-imidazol-5-yl)-N-[(2,2- |
| | difluoro-1,3-benzodioxol-5-yl)methyl]pentan-1-amine |
| 2501. | N-(1,3-benzodioxol-5-ylmethyl)-1-(1-butyl-2-phenyl-1H-imidazol-5-yl)-N-[4- |
| | (difluoromethoxy)benzyl]pentan-1-amine |
| 2502. | N-(1,3-benzodioxol-5-ylmethyl)-1-(1-butyl-2-phenyl-1H-imidazol-5-yl)-N-(3- |
| | ethoxybenzyl)pentan-1-amine |
| 2503. | N,N-bis(1,3-benzodioxol-5-ylmethyl)-1-(1-butyl-2-phenyl-1H-imidazol-5-yl)pentan-1- |
| | amine |
| 2504. | 4-({(1,3-benzodioxol-5-ylmethyl)[(1R)-1-(1-butyl-2,4-diphenyl-1H-imidazol-5- |
| | yl)pentyl]amino}methyl)benzenesulfonamide |
| 2505. | methyl 4-[(butyl{[1-butyl-2-phenyl-4-(trifluoromethyl)-1H-imidazol-5- |
| | yl]methyl}amino)methyl]benzoate |
| 2506. | 4-[(butyl{[1-butyl-2-phenyl-4-(trifluoromethyl)-1H-imidazol-5- |
| | yl]methyl}amino)methyl]benzoic acid |
| 2507. | 4-[(butyl{[1-butyl-2-phenyl-4-(trifluoromethyl)-1H-imidazol-5- |
| | yl]methyl}amino)methyl]benzamide |
| 2508. | 4-({{[1-butyl-2-phenyl-4-(trifluoromethyl)-1H-imidazol-5-yl]methyl}[3-(1,1,2,2- |
| | tetrafluoroethoxy)benzyl]amino}methyl)benzenesulfonamide |
| 2509. | 1-(1,3-benzodioxol-5-yl)-N-(1,3-benzodioxol-5-ylmethyl)-N-{[1-butyl-4- |
| | (diphenylphosphoryl)-2-phenyl-1H-imidazol-5-yl]methyl}methanamine |
| 2510. | 1-(1,3-benzodioxol-5-yl)-N-(1,3-benzodioxol-5-ylmethyl)-N-{[1-butyl-4-(2- |
| | methoxyphenyl)-2-phenyl-1H-imidazol-5-yl]methyl}methanamine |
| 2511. | 1-(1,3-benzodioxol-5-yl)-N-{[1-butyl-2-(2-methoxyphenyl)-4-(trifluoromethyl)-1H- |
| | imidazol-5-yl]methyl}-N-[4-(difluoromethoxy)benzyl]methanamine |
| 2512. | 4-{[{[1-butyl-2-phenyl-4-(trifluoromethyl)-1H-imidazol-5-yl]methyl}(3- |
| | ethoxybenzyl)amino]methyl}benzenesulfonamide |
| 2513. | 4-[((1,3-benzodioxol-5-ylmethyl) {[1-butyl-2-(2-methoxyphenyl)-4-(trifluoromethyl)- |
| | 1H-imidazol-5-yl]methyl}amino)methyl]benzenesulfonamide |
| 2514. | 4-[((1,3-benzodioxol-5-ylmethyl) {[1-butyl-2-(2-methoxyphenyl)-4-(trifluoromethyl)- |
| | 1H-imidazo1-5-yl]methyl}amino)methyl]-2,6-dimethylphenol |
| 2515. | 1-(1,3-benzodioxol-5-yl)-N-{[1-butyl-2-(2-methoxyphenyl)-4-(trifluoromethyl)-1H- |
| | imidazol-5-yl]methyl}-N-(3-ethoxybenzyl)methanamine |
| 2516. | 1-(1,3-benzodioxol-5-yl)-N-{[1-butyl-2-(2-methoxyphenyl)-4-(trifluoromethyl)-1H- |
| | imidazol-5-yl]methyl}-N-[4-(difluoromethoxy)-3-fluorobenzyl]methanamine |
| 2517. | 1-(1,3-benzodioxol-5-yl)-N-{[1-butyl-2-(2-methoxyphenyl)-4-(trifluoromethyl)-1H- |
| | imidazol-5-yl]methyl}-N-[3-(difluoromethoxy)benzyl]methanamine |
| 2518. | N-benzyl-1-(1-butyl-2,4-diphenyl-1H-imidazol-5-yl)-N-(2,3- |
| | difluorobenzyl)methanamine |
| 2519. | N-[(1-butyl-2,4-diphenyl-1H-imidazo1-5-yl)methyl]-N-(2,3-difluorobenzyl)butan-1- |
| | amine |
| 2520. | 1-(1-butyl-2,4-diphenyl-1H-imidazol-5-yl)-N-(cyclohexylmethyl)-N-(2,3- |
| | difluorobenzyl)methanamine |
| 2521. | 1-(1,3-benzodioxol-5-yl)-N-[(1-butyl-2,4-diphenyl-1H-imidazol-5-yl)methyl]-N-(2,3- |
| | difluorobenzyl)methanamine |
| 2522. | 1-(1-butyl-2,4-diphenyl-1H-imidazol-5-yl)-N-(2,3-difluorobenzyl)-N-[3- |
| | (trifluoromethyl)benzyl]methanamine |
| 2523. | 1-(1-butyl-2,4-diphenyl-1H-imidazol-5-yl)-N-(2,3-difluorobenzyl)-N-[4- |
| | (difluoromethoxy)benzyl]methanamine |
| 2524. | N-benzyl-1-(1-butyl-2,4-diphenyl-1H-imidazol-5-yl)-N-(3,5- |
| | difluorobenzyl)methanamine |
| 2525. | N-[(1-butyl-2,4-diphenyl-1H-imidazol-5-yl)methyl]-N-(3,5-difluorobenzyl)butan-1- |
| | amine |
| 2526. | 1-(1-butyl-2,4-diphenyl-1H-imidazol-5-yl)-N-(cyclohexylmethyl)-N-(3,5- |
| | difluorobenzyl)methanamine |
| 2527. | 1-(1,3-benzodioxol-5-yl)-N-[(1-butyl-2,4-diphenyl-1H-imidazol-5-yl)methyl]-N-(3,5- |
| | difluorobenzyl)methanamine |
| 2528. | 1-(1-butyl-2,4-diphenyl-1H-imidazol-5-yl)-N-(3,5-difluorobenzyl)-N-[3- |
| | (trifluoromethyl)benzyl]methanamine |
| 2529. | 1-(1-butyl-2,4-diphenyl-1H-imidazol-5-yl)-N-(3,5-difluorobenzyl)-N-[4- |
| | (difluoromethoxy)benzyl]methanamine |
| 2530. | 1-(1-butyl-2,4-diphenyl-1H-imidazo1-5-yl)-N-(3-chloro-4-fluorobenzyl)-N- |
| | (cyclohexylmethyl)methanamine |
| 2531. | 1-(1,3-benzodioxol-5-yl)-N-[(1-butyl-2,4-diphenyl-1H-imidazol-5-yl)methyl]-N-(3- |
| | chloro-4-fluorobenzyl)methanamine |
| 2532. | 1-(1-butyl-2,4-diphenyl-1H-imidazol-5-yl)-N-(3-chloro-4-fluorobenzyl)-N-[4- |
| | (difluoromethoxy)benzyl]methanamine |
| 2533. | N-benzyl-1-(1-butyl-2,4-diphenyl-1H-imidazol-5-yl)-N-(4-chloro-3- |
| | fluorobenzyl)methanamine |
| 2534. | N-[(1-butyl-2,4-diphenyl-1H-imidazo1-5-yl)methyl]-N-(4-chloro-3-fluorobenzyl)butan- |
| | 1-amine |
| 2535. | 1-(1-butyl-2,4-diphenyl-1H-imidazol-5-yl)-N-(4-chloro-3-fluorobenzyl)-N- |
| | (cyclohexylmethyl)methanamine |
| 2536. | 1-(1,3-benzodioxol-5-yl)-N-[(1-butyl-2,4-diphenyl-1H-imidazol-5-yl)methyl]-N-(4- |
| | chloro-3-fluorobenzyl)methanamine |
| 2537. | 1-(1-butyl-2,4-diphenyl-1H-imidazol-5-yl)-N-(4-chloro-3-fluorobenzyl)-N-[3- |
| | (trifluoromethyl)benzyl]methanamine |
| 2538. | N-[(1-butyl-2,4-diphenyl-1H-imidazol-5-yl)methyl]-N-(2-chloro-4-fluorobenzyl)butan- |
| | 1-amine |
| 2539. | 1-(1-butyl-2,4-diphenyl-1H-imidazol-5-yl)-N-(2-chloro-4-fluorobenzyl)-N- |
| | (cyclohexylmethyl)methanamine |
| 2540. | 1-(1,3-benzodioxol-5-yl)-N-[(1-butyl-2,4-diphenyl-1H-imidazol-5-yl)methyl]-N-(2- |
| | chloro-4-fluorobenzyl)methanamine |
| 2541. | 1-(1-butyl-2,4-diphenyl-1H-imidazol-5-yl)-N-(2-chloro-4-fluorobenzyl)-N-[3- |
| | (trifluoromethyl)benzyl]methanamine |
| 2542. | 1-(1-butyl-2,4-diphenyl-1H-imidazol-5-yl)-N-(2-chloro-4-fluorobenzyl)-N-[4- |
| | (difluoromethoxy)benzyl]methanamine |
| 2543. | N-benzyl-1-(3-bromo-4-fluorophenyl)-N-[(1-butyl-2,4-diphenyl-1H-imidazol-5- |
| | yl)methyl]methanamine |
| 2544. | N-(3-bromo-4-fluorobenzyl)-N-[(1-butyl-2,4-diphenyl-1H-imidazol-5-yl)methyl]butan- |
| | 1-amine |
| 2545. | 1-(3-bromo-4-fluorophenyl)-N-[(1-butyl-2,4-diphenyl-1H-imidazol-5-yl)methyl]-N- |
| | (cyclohexylmethyl)methanamine |
| 2546. | 1-(1,3-benzodioxol-5-yl)-N-(3-bromo-4-fluorobenzyl)-N-[(1-butyl-2,4-diphenyl-1H- |
| | imidazol-5-yl)methyl]methanamine |
| 2547. | 1-(3-bromo-4-fluorophenyl)-N-[(1-butyl-2,4-diphenyl-1H-imidazol-5-yl)methyl]-N-[4- |
| | (difluoromethoxy)benzyl]methanamine |
| 2548. | 3-(5-{[bis(1,3-benzodioxol-5-ylmethyl)amino]methyl}-1-butyl-2-phenyl-1H-imidazol- |
| | 4-yl)phenol |
| 2549. | 3-(5-{[bis(1,3-benzodioxol-5-ylmethyl)amino]methyl}-1-butyl-4-phenyl-1H-imidazol- |
| | 2-yl)phenol |
| 2550. | 4-[((1,3-benzodioxol-5-ylmethyl){[1-butyl-2-(2-methoxyphenyl)-4-(trifluoromethyl)- |
| | 1H-imidazol-5-yl]methyl}amino)methyl]-3-chlorophenol |
| 2551. | 1-(1,3-benzodioxol-5-yl)-N-(1,3-benzodioxol-5-ylmethyl)-N-[(1-butyl-2-phenyl-4- |
| | thien-3-yl-1H-imidazol-5-yl)methyl]methanamine |
| 2552. | methyl 4-{[{[1-butyl-2-phenyl-4-(trifluoromethyl)-1H-imidazol-5- |
| | yl]methyl}(isopentyl)amino]methyl}benzoate |
| 2553. | methyl 4-{[{[1-butyl-2-phenyl-4-(trifluoromethyl)-1H-imidazol-5- |
| | yl]methyl}(isobutyl)amino]methyl}benzoate |
| 2554. | methyl 4-{[{[1-butyl-2-phenyl-4-(trifluoromethyl)-1H-imidazol-5- |
| | yl]methyl}(cyclohexylmethyl)amino]methyl}benzoate |
| 2555. | 4-{[{[1-butyl-2-phenyl-4-(trifluoromethyl)-1H-imidazol-5- |
| | yl]methyl}(isopentyl)amino]methyl}benzoic acid |
| 2556. | 4-{[{[1-butyl-2-phenyl-4-(trifluoromethyl)-1H-imidazol-5- |
| | yl]methyl}(isobutyl)amino]methyl}benzoic acid |
| 2557. | 4-{[{[1-butyl-2-phenyl-4-(trifluoromethyl)-1H-imidazol-5- |
| | yl]methyl}(cyclohexylmethyl)amino]methyl}benzoic acid |
| 2558. | 4-{[{[1-butyl-2-phenyl-4-(trifluoromethyl)-1H-imidazol-5- |
| | yl]methyl}(cyclohexylmethyl)amino]methyl}benzamide |
| 2559. | 4-{[{[1-butyl-2-phenyl-4-(trifluoromethyl)-1H-imidazol-5- |
| | yl]methyl}(isobutyl)amino]methyl}benzamide |
| 2560. | 4-{[{[1-butyl-2-phenyl-4-(trifluoromethyl)-1H-imidazol-5- |
| | yl]methyl}(isopentyl)amino]methyl}benzamide |
| 2561. | 1-(1,3-benzodioxol-5-yl)-N-(1,3-benzodioxol-5-ylmethyl)-N-{[1-butyl-4- |
| | (pentafluoroethyl)-2-phenyl-1H-imidazol-5-yl]methyl}methanamine |
| 2562. | 1-(1,3-benzodioxol-5-yl)-N-{[1-butyl-4-(pentafluoroethyl)-2-phenyl-1H-imidazol-5- |
| | yl]methyl}-N-[3-(1,1,2,2-tetrafluoroethoxy)benzyl]methanamine |
| 2563. | 1-(1,3-benzodioxol-5-yl)-N-{[1-butyl-4-(pentafluoroethyl)-2-phenyl-1H-imidazol-5- |
| | yl]methyl}-N-[4-(difluoromethoxy)benzyl]methanamine |
| 2564. | 1-(1,3-benzodioxol-5-yl)-N-{[1-butyl-4-(pentafluoroethyl)-2-phenyl-1H-imidazol-5- |
| | yl]methyl}-N-(3-ethoxybenzyl)methanamine |
| 2565. | 1-(1,3-benzodioxol-5-yl)-N-{[1-butyl-4-(pentafluoroethyl)-2-phenyl-1H-imidazol-5- |
| | yl]methyl}-N-[3-(difluoromethoxy)benzyl]methanamine |
| 2566. | methyl 4-( {{[1-butyl-2-phenyl-4-(trifluoromethyl)-1H-imidazol-5-yl]methyl}[3- |
| | (difluoromethoxy)benzyl]amino}methyl)benzoate |
| 2567. | 1-(1-butyl-2-phenyl-1H-imidazol-5-yl)-N-[4-(difluoromethoxy)benzyl]-N-(3- |
| | ethoxybenzyl)methanamine |
| 2568. | 1-[4-({benzyl [(1-butyl-2,4-diphenyl-1H-imidazol-5- |
| | yl)methyl]amino}methyl)phenyl]ethanone |
| 2569. | 1-[4-({butyl[(1-butyl-2,4-diphenyl-1H-imidazol-5- |
| | yl)methyl]amino}methyl)phenyl]ethanone |
| 2570. | 1-[4-({(1,3-benzodioxol-5-ylmethyl)[(1-butyl-2,4-diphenyl-1H-imidazol-5- |
| | yl)methyl]amino}methyl)phenyl]ethanone |
| 2571. | 1-[4-({[(1-butyl-2,4-diphenyl-1H-imidazol-5-yl)methyl]4- |
| | (difluoromethoxy)benzyl]amino}methyl)phenyl]ethanone |
| 2572. | N-[4-({benzyl[(1-butyl-2,4-diphenyl-1H-imidazol-5- |
| | yl)methyl]amino}methyl)phenyl]acetamide |
| 2573. | N-[4-({butyl[(1-butyl-2,4-diphenyl-1H-imidazol-5- |
| | yl)methyl]amino}methyl)phenyl]acetamide |
| 2574. | N-[4-({(1,3-benzodioxol-5-ylmethyl)[(1-butyl-2,4-diphenyl-1H-imidazol-5- |
| | yl)methyl]amino}methyl)phenyl]acetamide |
| 2575. | N-[4-({[(1-butyl-2,4-diphenyl-1H-imidazol-5-yl)methyl][4- |
| | (difluoromethoxy)benzyl]amino}methyl)phenyl]acetamide |
| 2576. | N-benzyl-1-(1-butyl-2,4-diphenyl-1H-imidazol-5-yl)-N-[(6-methylpyridin-2- |
| | yl)methyl]methanamine |
| 2577. | N-[(1-butyl-2,4-diphenyl-1H-imidazol-5-yl)methyl]-N-[(6-methylpyridin-2- |
| | yl)methyl]butan-1-amine |
| 2578. | 1-(1,3-benzodioxol-5-yl)-N-[(1-butyl-2,4-diphenyl-1H-imidazol-5-yl)methyl]-N-[(6- |
| | methylpyridin-2-yl)methyl]methanamine |
| 2579. | 1-(1-butyl-2,4-diphenyl-1H-imidazol-5-yl)-N-[4-(difluoromethoxy)benzyl]-N-[(6- |
| | methylpyridin-2-yl)methyl]methanamine |
| 2580. | N-[(4-bromo-1-butyl-2-phenyl-1H-imidazol-5-yl)methyl]-N-[4- |
| | (difluoromethoxy)benzyl]-N-(3-ethoxybenzyl)amine |
| 2581. | 1-(1,3-benzodioxol-5-yl)-N-(1,3-benzodioxol-5-ylmethyl)-N-({1-butyl-4-[4- |
| | (methylthio)phenyl]-2-phenyl-1H-imidazol-5-yl}methyl)methanamine |
| 2582. | 1-(1,3-benzodioxol-5-yl)-N-(1,3-benzodioxol-5-ylmethyl)-N-({1-butyl-4-[4- |
| | (methylsulfonyl)phenyl]-2-phenyl-1H-imidazol-5-yl}methyl)methanamine |
| 2583. | 1-[1-butyl-2-phenyl-4-(trifluoromethyl)-1H-imidazol-5-yl]-N-[4- |
| | (difluoromethoxy)benzyl]-N-[4-(methylthio)benzyl]methanamine |
| 2584. | 4-({{[1-butyl-2-phenyl-4-(trifluoromethyl)-1H-imidazol-5-yl]methyl}[4- |
| | (methylthio)benzyl]amino}methyl)benzenesulfonamide |
| 2585. | 1-(1,3-benzodioxol-5-yl)-N-{[1-butyl-2-(2-methoxyphenyl)-4-(trifluoromethyl)-1H- |
| | imidazol-5-yl]methyl}-N-[4-(methylthio)benzyl]methanamine |
| 2586. | 1-[1-butyl-2-(2-methoxyphenyl)-4-(trifluoromethyl)-1H-imidazol-5-yl]-N-[4- |
| | (difluoromethoxy)benzyl]-N-[4-(methylthio)benzyl]methanamine |
| 2587. | 1-(1-butyl-2,4-diphenyl-1H-imidazol-5-yl)-N-[3-(difluoromethoxy)benzyl]-N-[4- |
| | (methylthio)benzyl]methanamine |
| 2588. | 1-(1,3-benzodioxol-5-yl)-N-{[1-butyl-2-phenyl-4-(trifluoromethyl)-1H-imidazol-5- |
| | yl]methyl}-N-[4-(methylsulfonyl)benzyl]methanamine |
| 2589. | methyl 4-({{[1-butyl-2-phenyl-4-(trifluoromethyl)-1H-imidazol-5-yl]methyl}[4- |
| | (difluoromethoxy)benzyl]amino}methyl)benzoate |
| 2590. | methyl 4-[((1,3-benzodioxol-5-ylmethyl){[1-butyl-2-phenyl-4-(trifluoromethyl)-1H- |
| | imidazol-5-yl]methyl}amino)methyl]benzoate |
| 2591. | 4-[((1,3-benzodioxol-5-ylmethyl){[1-butyl-2-phenyl-4-(trifluoromethyl)-1H-imidazol- |
| | 5-yl]methyl}amino)methyl]benzoic acid |
| 2592. | 4-({{[1-butyl-2-phenyl-4-(trifluoromethyl)-1H-imidazol-5-yl]methyl}[4- |
| | (difluoromethoxy)benzyl]amino}methyl)benzoic acid |
| 2593. | 4-({{[1-butyl-2-phenyl-4-(trifluoromethyl)-1H-imidazol-5-yl]methyl}[4- |
| | (difluoromethoxy)benzyl]amino}methyl)benzamide |
| 2594. | 4-[((1,3-benzodioxol-5-ylmethyl){[1-butyl-2-phenyl-4-(trifluoromethyl)-1H-imidazol- |
| | 5-yl]methyl}amino)methyl]benzamide |
| 2595. | methyl 4-({{[1-butyl-2-phenyl-4-(trifluoromethyl)-1H-imidazol-5-yl]methyl}[3- |
| | (1,1,2,2-tetrafluoroethoxy)benzyl]amino}methyl)benzoate |
| 2596. | methyl 4-{[{[1-butyl-2-phenyl-4-(trifluoromethyl)-1H-imidazol-5-yl]methyl}(3- |
| | ethoxybenzyl)amino]methyl}benzoate |
| 2597. | 4-({{[1-butyl-2-phenyl-4-(trifluoromethyl)-1H-imidazol-5-yl]methyl}[3- |
| | (difluoromethoxy)benzyl]amino}methyl)benzoic acid |
| 2598. | 4-({{[1-butyl-2-phenyl-4-(trifluoromethyl)-1H-imidazol-5-yl]methyl}[3-(1,1,2,2- |
| | tetrafluoroethoxy)benzyl]amino}methyl)benzoic acid |
| 2599. | 4-{[{[1-butyl-2-phenyl-4-(trifluoromethyl)-1H-imidazol-5-yl]methyl}(3- |
| | ethoxybenzyl)amino]methyl)benzoic acid |
| 2600. | 4-({{[1-butyl-2-phenyl-4-(trifluoromethyl)-1H-imidazol-5-yl]methyl}[3- |
| | (trifluoromethyl)benzyl]amino}methyl)benzoic acid |
| 2601. | 4-({{[1-butyl-2-phenyl-4-(trifluoromethyl)-1H-imidazol-5-yl]methyl}[3- |
| | (difluoromethoxy)benzyl]amino}methyl)benzamide |
| 2602. | 4-{[{[1-butyl-2-phenyl-4-(trifluoromethyl)-1H-imidazol-5-yl]methyl}(3- |
| | ethoxybenzyl)amino]methyl}benzamide |
| 2603. | 4-({{[1-butyl-2-phenyl-4-(trifluoromethyl)-1H-imidazol-5-yl]methyl}[3-(1,1,2,2- |
| | tetrafluoroethoxy)benzyl]amino}methyl)benzamide |
| 2604. | 4-({{[1-butyl-2-phenyl-4-(trifluoromethyl)-1H-imidazol-5-yl]methyl}[3- |
| | (trifluoromethyl)benzyl]amino}methyl)benzamide |
| 2605. | 5-{[bis(1,3-benzodioxol-5-ylmethyl)amino]methyl}-1-butyl-2-phenyl-1H-imidazole-4- |
| | carbonitrile |
| 2606. | 1-(1,3-benzodioxol-5-yl)-N-(1,3-benzodioxol-5-ylmethyl)-N-[(1-butyl-2-phenyl-4- |
| | pyrazin-2-yl-1H-imidazol-5-yl)methyl]methanamine |
| 2607. | 1-(1,3-benzodioxol-5-yl)-N-(1,3-benzodioxol-5-ylmethyl)-N-{[1-butyl-2-phenyl-4-(1,3- |
| | thiazol-2-yl)-1H-imidazol-5-yl]methyl}methanamine |
| 2608. | 1-(1,3-benzodioxol-5-yl)-N-(1,3-benzodioxol-5-ylmethyl)-N-{[1-butyl-2-phenyl-4- |
| | (phenylethynyl)-1H-imidazol-5-yl]methyl}methanamine |
| 2609. | 1-(1,3-benzodioxol-5-yl)-N-(1,3-benzodioxol-5-ylmethyl)-N-({1-butyl-4-[(1E)-pent-1- |
| | enyl]-2-phenyl-1H-imidazol-5-yl}methyl)methanamine |
| 2610. | 1-(1,3-benzodioxol-5-yl)-N-(1,3-benzodioxol-5-ylmethyl)-N-[(1-butyl-2-phenyl-4- |
| | vinyl-1H-imidazol-5-yl)methyl]methanamine |
| 2611. | 1-(1,3-benzodioxol-5-yl)-N-(1,3-benzodioxol-5-ylmethyl)-N-[(1-butyl-2-phenyl-4- |
| | pyridin-2-yl-1H-imidazol-5-yl)methyl]methanamine |
| 2612. | 1-[4-({{[1-butyl-2-phenyl-4-(trifluoromethyl)-1H-imidazol-5-yl]methyl}[4- |
| | (difluoromethoxy)benzyl]amino}methyl)phenyl]ethanone |
| 2613. | 1-{4-[((1,3-benzodioxol-5-ylmethyl){[1-butyl-2-phenyl-4-(trifluoromethyl)-1H- |
| | imidazol-5-yl]methyl}amino)methyl]phenyl}ethanone |
| 2614. | 4-[((4-acetylbenzyl){[1-butyl-2-phenyl-4-(trifluoromethyl)-1H-imidazol-5- |
| | yl]methyl}amino)methyl]benzenesulfonamide |
| 2615. | N,N-bis(1,3-benzodioxol-5-ylmethyl)-1-[1-butyl-2-phenyl-4-(trifluoromethyl)-1H- |
| | imidazol-5-yl]ethanamine |
| 2616. | 1-(1,3-benzodioxol-5-yl)-N-(1,3-benzodioxol-5-ylmethyl)-N-{[1-butyl-4-(1-naphthyl)- |
| | 2-phenyl-1H-imidazol-5-yl]methyl}methanamine |
| 2617. | 1-(1,3-benzodioxol-5-yl)-N-(1,3-benzodioxol-5-ylmethyl)-N-[(1-butyl-2-phenyl-4- |
| | pyridin-3-yl-1H-imidazol-5-yl)methyl]methanamine |
| 2618. | 1-(1,3-benzodioxol-5-yl)-N-(1,3-benzodioxol-5-ylmethyl)-N-{[1-butyl-4-(3- |
| | ethoxyphenyl)-2-phenyl-1H-imidazol-5-yl]methyl}methanamine |
| 2619. | 1-(1,3-benzodioxol-5-yl)-N-(1,3-benzodioxol-5-ylmethyl)-N-{[1-butyl-4-(5- |
| | methylthien-2-yl)-2-phenyl-1H-imidazol-5-yl]methyl}methanamine |
| 2620. | 1-(1,3-benzodioxol-5-yl)-N-(1,3-benzodioxol-5-ylmethyl)-N-({1-butyl-2-phenyl-4-[2- |
| | (trifluoromethyl)phenyl]-1H-imidazol-5-yl}methyl)methanamine |
| 2621. | 5-({(1,3-benzodioxol-5-ylmethyl)[4-(difluoromethoxy)benzyl]amino}methyl)-1-butyl- |
| | 2-phenyl-1H-imidazole-4-carbonitrile |
| 2622. | 4-({(1,3-benzodioxol-5-ylmethyl)[(1-butyl-4-cyano-2-phenyl-1H-imidazol-5- |
| | yl)methyl]amino}methyl)benzenesulfonamide |
| 2623. | 4-({(1,3-benzodioxol-5-ylmethyl)[(1-butyl-4-cyano-2-phenyl-1H-imidazol-5- |
| | yl)methyl]amino}methyl)benzamide |
| 2624. | 5-{[(1,3-benzodioxol-5-ylmethyl)(3-ethoxybenzyl)amino]methyl}-1-butyl-2-phenyl- |
| | 1H-imidazole-4-carbonitrile |
| 2625. | 5-({(1,3-benzodioxol-5-ylmethyl)[3-(1,1,2,2-tetrafluoroethoxy)benzyl]amino}methyl)- |
| | 1-butyl-2-phenyl-1H-imidazole-4-carbonitrile |
| 2626. | 5-({(1,3-benzodioxol-5-ylmethyl)[3-(difluoromethoxy)benzyl]amino}methyl)-1-butyl- |
| | 2-phenyl-1H-imidazole-4-carbonitrile |
| 2627. | 1-(1,3-benzodioxol-5-yl)-N-(1,3-benzodioxol-5-ylmethyl)-N-[( 1-butyl-4-methyl-2- |
| | phenyl-1H-imidazol-5-yl)methyl]methanamine |
| 2628. | 1-(1,3-benzodioxol-5-yl)-N-(1,3-benzodioxol-5-ylmethyl)-N-[(1-butyl-4-ethyl-2- |
| | phenyl-1H-imidazol-5-yl)methyl]methanamine |
| 2629. | N-[(1-butyl-4-chloro-2-phenyl-1H-imidazol-5-yl)methyl]-N-[4- |
| | (difluoromethoxy)benzyl]-N-(3-ethoxybenzyl)amine |
| 2630. | 5-({butyl[(1-butyl-2,4-diphenyl-1H-imidazol-5-yl)methyl]amino}methyl)-2- |
| | (methylamino)benzenethiol |
| 2631. | 5-({benzyl[(1-butyl-2,4-diphenyl-1H-imidazol-5-yl)methyl]amino}methyl)-2- |
| | (methylamino)benzenethiol |
| 2632. | 1-(1,3-benzodioxol-5-yl)-N-(1,3-benzodioxol-5-ylmethyl)-N-{[1-butyl-4-(4- |
| | ethoxyphenyl)-2-phenyl-1H-imidazol-5-yl]methyl}methanamine |
| 2633. | ethyl (2E)-3-(5-{[bis(1,3-benzodioxol-5-ylmethyl)amino]methyl}-1-butyl-2-phenyl-1H- |
| | imidazol-4-yl)prop-2-enoate |
| 2634. | 1-(1,3-benzodioxol-5-yl)-N-(1,3-benzodioxol-5-ylmethyl)-N-[(1-butyl-2-phenyl-4- |
| | thien-2-yl-1H-imidazol-5-yl)methyl]methanamine |
| 2635. | 1-[5-(5-{[bis(1,3-benzodioxol-5-ylmethyl)amino]methyl}-1-butyl-2-phenyl-1H- |
| | imidazol-4-yl)thien-2-yl]ethanone |
| 2636. | 6-({butyl[(1-butyl-2,4-diphenyl-1H-imidazol-5-yl)methyl]amino}methyl)-3-methyl-1,3- |
| | benzothiazol-2(3H)-one |
| 2637. | 1-(1-butyl-2-phenyl-4-thien-3-yl-1H-imidazol-5-yl)-N,N-bis(3- |
| | ethoxybenzyl)methanamine |
| 2638. | 1-(1-butyl-2-phenyl-4-thien-3-yl-1H-imidazol-5-yl)-N,N-bis[4- |
| | (difluoromethoxy)benzyl]methanamine |
| 2639. | 2-(aminocarbonyl)-5-[(butyl{[1-butyl-4-(4-methoxyphenyl)-2-phenyl-1H-imidazol-5- |
| | yl]methyl}amino)methyl]phenyl acetate |
| 2640. | 4-[(butyl{[1-butyl-4-(4-methoxyphenyl)-2-phenyl-1H-imidazol-5- |
| | yl]methyl}amino)methyl]-2-hydroxybenzamide |
| 2641. | 2-(aminocarbonyl)-5-{[{[1-butyl-4-(4-methoxyphenyl)-2-phenyl-1H-imidazol-5- |
| | yl]methyl}(isopentyl)amino]methyl}phenyl acetate |
| 2642. | 4-{[{[1-butyl-4-(4-methoxyphenyl)-2-phenyl-1H-imidazol-5- |
| | yl]methyl}(isopentyl) amino]methyl}-2-hydroxybenzamide |
| 2643. | 2-(aminocarbonyl)-5-{[{[1-butyl-4-(4-methoxyphenyl)-2-phenyl-1H-imidazol-5- |
| | yl]methyl}(cyclohexylmethyl)amino]methyl}phenyl acetate |
| 2644. | 4-{[{[1-butyl-4-(4-methoxyphenyl)-2-phenyl-1H-imidazol-5- |
| | yl]methyl}(cyclohexylmethyl)amino]methyl}-2-hydroxybenzamide |
| 2645. | 2-(aminocarbonyl)-5-[((1,3-benzodioxol-5-ylmethyl){[1-butyl-4-(4-methoxyphenyl)-2- |
| | phenyl-1H-imidazol-5-yl]methyl}amino)methyl]phenyl acetate |
| 2646. | 4-[((1,3-benzodioxol-5-ylmethyl){[1-butyl-4-(4-methoxyphenyl)-2-phenyl-1H- |
| | imidazol-5-yl]methyl}amino)methyl]-2-hydroxybenzamide |
| 2647. | ethyl 5-{[bis(1,3-benzodioxol-5-ylmethyl)amino]methyl}-1-butyl-2-phenyl-1H- |
| | imidazole-4-carboxylate |
| 2648. | 1-(1,3-benzodioxol-5-yl)-N-[(1-butyl-4-chloro-2-phenyl-1H-imidazol-5-yl)methyl]-N- |
| | [4-(difluoromethoxy)benzyl]methanamine |
| 2649. | 1-(1,3-benzodioxol-5-yl)-N-(1,3-benzodioxol-5-ylmethyl)-N-{[1-butyl-4- |
| | (methoxymethyl)-2-phenyl-1H-imidazol-5-yl]methyl}methanamine |
| 2650. | N-{[1-butyl-4-(4-methylphenyl)-2-phenyl-1H-imidazol-5-yl]methyl}-N-[4- |
| | (difluoromethoxy)benzyl]-N-(3-ethoxybenzyl)amine |
| 2651. | 1-(1,3-benzodioxol-5-yl)-N-{[1-butyl-4-(4-methylphenyl)-2-phenyl-1H-imidazol-5- |
| | yl]methyl}-N-[4-(difluoromethoxy)benzyl]methanamine |
| 2652. | N-{[1-butyl-4-(4-methylphenyl)-2-phenyl-1H-imidazol-5-yl]methyl}-N,N-bis(3- |
| | ethoxybenzyl)amine |
| 2653. | N-{[1-butyl-4-(4-methylphenyl)-2-phenyl-1H-imidazol-5-yl]methyl}-N,N-bis[4- |
| | (difluoromethoxy)benzyl]amine |
| 2654. | N-{[1-butyl-4-(3-methylphenyl)-2-phenyl-1H-imidazol-5-yl]methyl}-N-[4- |
| | (difluoromethoxy)benzyl]-N-(3-ethoxybenzyl)amine |
| 2655. | 1-(1,3-benzodioxol-5-yl)-N-{[1-butyl-4-(3-methylphenyl)-2-phenyl-1H-imidazol-5- |
| | yl]methyl}-N-[4-(difluoromethoxy)benzyl]methanamine |
| 2656. | N-{[1-butyl-4-(3-methylphenyl)-2-phenyl-1H-imidazol-5-yl]methyl}-N,N-bis(3- |
| | ethoxybenzyl)amine |
| 2657. | N-{[1-butyl-4-(3-methylphenyl)-2-phenyl-1H-imidazol-5-yl]methyl}-N,N-bis[4- |
| | (difluoromethoxy)benzyl]amine |
| 2658. | N-{[1-butyl-4-(4-fluorophenyl)-2-phenyl-1H-imidazol-5-yl]methyl}-N-[4- |
| | (difluoromethoxy)benzyl]-N-(3-ethoxybenzyl)amine |
| 2659. | 1-(1,3-benzodioxol-5-yl)-N-{[1-butyl-4-(4-fluorophenyl)-2-phenyl-1H-imidazol-5- |
| | yl]methyl}-N-[4-(difluoromethoxy)benzyl]methanamine |
| 2660. | N-{[1-butyl-4-(4-fluorophenyl)-2-phenyl-1H-imidazol-5-yl]methyl}-N,N-bis(3- |
| | ethoxybenzyl)amine |
| 2661. | N-{[1-butyl-4-(4-fluorophenyl)-2-phenyl-1H-imidazol-5-yl]methyl}-N,N-bis[4- |
| | (difluoromethoxy)benzyl]amine |
| 2662. | 5-{[bis(1,3-benzodioxol-5-ylmethyl)amino]methyl}-1-butyl-N,N-dimethyl-2-phenyl- |
| | 1H-imidazole-4-carboxamide |
| 2663. | isopropyl 5-{[bis(1,3-benzodioxol-5-ylmethyl)amino]methyl}-1-butyl-2-phenyl-1H- |
| | imidazole-4-carboxylate |
| 2664. | 1-(1,3-benzodioxol-5-yl)-N-(1,3-benzodioxol-5-ylmethyl)-N-{[1-butyl-4- |
| | (ethoxymethyl)-2-phenyl-1H-imidazol-5-yl]methyl}methanamine |
| 2665. | 1-(1,3-benzodioxol-5-yl)-N-(1,3-benzodioxol-5-ylmethyl)-N-{[1-butyl-4-(3,5- |
| | dimethylphenyl)-2-phenyl-1H-imidazol-5-yl]methyl}methanamine |
| 2666. | 1-(1,3-benzodioxol-5-yl)-N-(1,3-benzodioxol-5-ylmethyl)-N-({[1-butyl-2-phenyl-4-[3- |
| | (trifluoromethyl)phenyl]-1H-imidazol-5-yl}methyl)methanamine |
| 2667. | 1-(1,3-benzodioxol-5-yl)-N-(1,3-benzodioxol-5-ylmethyl)-N-({[1-butyl-2-phenyl-4-[3- |
| | (trifluoromethoxy)phenyl]-1H-imidazol-5-yl}methyl)methanamine |
| 2668. | 7-[(butyl{[1-butyl-4-(3-methoxyphenyl)-2-phenyl-1H-imidazol-5- |
| | yl]methyl}amino)methyl]-2,3-dihydroquinazolin-4(1H)-one |
| 2669. | 1-(1,3-benzodioxol-5-yl)-N-[(1-butyl-2,4-diphenyl-1H-imidazol-5-yl)methyl]-N-[(1- |
| | methyl-1H-indol-5-yl)methyl]methanamine |
| 2670. | 1-[1-butyl-2-phenyl-4-(trifluoromethyl)-1H-imidazol-5-yl]-N-(cyclohexylmethyl)-N- |
| | (1H-indol-5-ylmethyl)methanamine |
| 2671. | 1-(1,3-benzodioxol-5-yl)-N-[(1-butyl-2,4-diphenyl-1H-imidazol-5-yl)methyl]-N-[(1- |
| | methyl-2,3-dihydro-1H-indol-5-yl)methyl]methanamine |
| 2672. | N-benzyl-1-(1-butyl-2,4-diphenyl-1H-imidazol-5-yl)-N-[(1-methyl-1H-indol-5- |
| | yl)methyl]methanamine |
| 2673. | 1-(1-butyl-2,4-diphenyl-1H-imidazol-5-yl)-N-[4-(difluoromethoxy)benzyl]-N-[(1- |
| | methyl-2,3-dihydro-1H-indol-5-yl)methyl]methanamine |
| 2674. | 1-(1,3-benzodioxol-5-yl)-N-{[1-butyl-2-phenyl-4-(trifluoromethyl)-1H-imidazol-5- |
| | yl]methyl}-N-(1H-indol-5-ylmethyl)methanamine |
| 2675. | N-{[1-butyl-2-phenyl-4-(trifluoromethyl)-1H-imidazol-5-yl]methyl}-N-(1H-indol-5- |
| | ylmethyl)butan-1-amine |
| 2676. | N-{[1-butyl-2-phenyl-4-(trifluoromethyl)-1H-imidazol-5-yl]methyl}-N-[(1-methyl-1H- |
| | indol-5-yl)methyl]butan-1-amine |
| 2677. | 1-[1-butyl-2-phenyl-4-(trifluoromethyl)-1H-imidazol-5-yl]-N-(cyclohexylmethyl)-N- |
| | [(1-methyl-1H-indol-5-yl)methyl]methanamine |
| 2678. | 1-(1-butyl-2,4-diphenyl-1H-imidazol-5-yl)-N-[4-(difluoromethoxy)benzyl]-N-[(1- |
| | methyl-1H-indol-5-yl)methyl]methanamine |
| 2679. | 1-(1,3-benzodioxol-5-yl)-N-{[1-butyl-2-phenyl-4-(trifluoromethyl)-1H-imidazol-5- |
| | yl]methyl}-N-[(1-methyl-1H-indol-5-yl)methyl]methanamine |
| 2680. | 1-(1-butyl-2,4-diphenyl-1H-imidazol-5-yl)-N-[4-(difluoromethoxy)benzyl]-N-(1H- |
| | indol-5-ylmethyl)methanamine |
| 2681. | 1-(1,3-benzodioxol-5-yl)-N-(2,1,3-benzoxadiazol-5-ylmethyl)-N-{[1-butyl-2-phenyl-4- |
| | (trifluoromethyl)-1H-imidazol-5-yl]methyl}methanamine |
| 2682. | 1-(1,3-benzodioxol-5-yl)-N-(2,1,3-benzoxadiazol-5-ylmethyl)-N-{[1-butyl-4-(3- |
| | methoxyphenyl)-2-phenyl-1H-imidazol-5-yl]methyl}methanamine |
| 2683. | 1-(1,3-benzodioxol-5-yl)-N-[(1-butyl-2-phenyl-1H-imidazol-5-yl)methyl]-N-(3- |
| | ethoxybenzyl)methanamine |
| 2684. | 1-(1,3-benzodioxol-5-yl)-N-[(1-butyl-2-phenyl-1H-imidazol-5-yl)methyl]-N-[3- |
| | (1,1,2,2-tetrafluoroethoxy)benzyl]methanamine |
| 2685. | 1-(1,3-benzodioxol-5-yl)-N-[(1-butyl-4-ethoxy-2-phenyl-1H-imidazol-5-yl)methyl]-N- |
| | (3-ethoxybenzyl)methanamine |
| 2686. | 1-(1,3-benzodioxol-5-yl)-N-[(4-bromo-1-butyl-2-phenyl-1H-imidazol-5-yl)methyl]-N- |
| | (3-ethoxybenzyl)methanamine |
| 2687. | 1-(1,3-benzodioxol-5-yl)-N-[(4-bromo-1-butyl-2-phenyl-1H-imidazol-5-yl)methyl]-N- |
| | [3-(1,1,2,2-tetrafluoroethoxy)benzyl]methanamine |
| 2688. | 1-(5-{[bis(1,3-benzodioxol-5-ylmethyl)amino]methyl}-1-butyl-2-phenyl-1H-imidazol- |
| | 4-yl)ethanol |
| 2689. | N-{[1-butyl-4-(3-fluorophenyl)-2-phenyl-1H-imidazol-5-yl]methyl}-N-[4- |
| | (difluoromethoxy)benzyl]-N-(3-ethoxybenzyl)amine |
| 2690. | 1-(1,3-benzodioxol-5-yl)-N-{[1-butyl-4-(3-fluorophenyl)-2-phenyl-1H-imidazol-5- |
| | yl]methyl}-N-[4-(difluoromethoxy)benzyl]methanamine |
| 2691. | N-{[1-butyl-4-(3-fluorophenyl)-2-phenyl-1H-imidazol-5-yl]methyl}-N,N-bis(3- |
| | ethoxybenzyl)amine |
| 2692. | N-{[1-butyl-4-(3-fluorophenyl)-2-phenyl-1H-imidazol-5-yl]methyl}-N,N-bis[4- |
| | (difluoromethoxy)benzyl]amine |
| 2693. | N-{[1-butyl-4-(4-ethoxyphenyl)-2-phenyl-1H-imidazol-5-yl]methyl}-N-[4- |
| | (difluoromethoxy)benzyl]-N-(3-ethoxybenzyl)amine |
| 2694. | 1-(1,3-benzodioxol-5-yl)-N-{[1-butyl-4-(4-ethoxyphenyl)-2-phenyl-1H-imidazol-5- |
| | yl]methyl}-N-[4-(difluoromethoxy)benzyl]methanamine |
| 2695. | N-{[1-butyl-4-(4-ethoxyphenyl)-2-phenyl-1H-imidazol-5-yl]methyl}-N,N-bis(3- |
| | ethoxybenzyl)amine |
| 2696. | N-{[1-butyl-4-(4-ethoxyphenyl)-2-phenyl-1H-imidazol-5-yl]methyl}-N,N-bis[4- |
| | (difluoromethoxy)benzyl]amine |
| 2697. | 1-[1-butyl-4-(3,4-difluorophenyl)-2-phenyl-1H-imidazol-5-yl]-N-[4- |
| | (difluoromethoxy)benzyl]-N-(3-ethoxybenzyl)methanamine |
| 2698. | 1-(1,3-benzodioxol-5-yl)-N-{[1-butyl-4-(3,4-difluorophenyl)-2-phenyl-1H-imidazol-5- |
| | yl]methyl}-N-[4-(difluoromethoxy)benzyl]methanamine |
| 2699. | 1-[1-butyl-4-(3,4-difluorophenyl)-2-phenyl-1H-imidazol-5-yl]-N,N-bis(3- |
| | ethoxybenzyl)methanamine |
| 2700. | 1-[1-butyl-4-(3,4-difluorophenyl)-2-phenyl-1H-imidazol-5-yl]-N,N-bis[4- |
| | (difluoromethoxy)benzyl]methanamine |
| 2701. | N-{[1-butyl-4-(3-chlorophenyl)-2-phenyl-1H-imidazol-5-yl]methyl}-N-[4- |
| | (difluoromethoxy)benzyl]-N-(3-ethoxybenzyl)amine |
| 2702. | 1-(1,3-benzodioxol-5-yl)-N-{[1-butyl-4-(3-chlorophenyl)-2-phenyl-1H-imidazol-5- |
| | yl]methyl}-N-[4-(difluoromethoxy)benzyl]methanamine |
| 2703. | N-{[1-butyl-4-(3-chlorophenyl)-2-phenyl-1H-imidazol-5-yl]methyl}-N,N-bis(3- |
| | ethoxybenzyl)amine |
| 2704. | N-{[1-butyl-4-(3-chlorophenyl)-2-phenyl-1H-imidazol-5-yl]methyl}-N,N-bis[4- |
| | (difluoromethoxy)benzyl]amine |
| 2705. | N-{[1-butyl-4-(4-chlorophenyl)-2-phenyl-1H-imidazol-5-yl]methyl}-N-[4- |
| | (difluoromethoxy)benzyl]-N-(3-ethoxybenzyl)amine |
| 2706. | 1-(1,3-benzodioxol-5-yl)-N-{[1-butyl-4-(4-chlorophenyl)-2-phenyl-1H-imidazol-5- |
| | yl]methyl}-N-[4-(difluoromethoxy)benzyl]methanamine |
| 2707. | N-{[1-butyl-4-(4-chlorophenyl)-2-phenyl-1H-imidazol-5-yl]methyl}-N,N-bis(3- |
| | ethoxybenzyl)amine |
| 2708. | N-{[1-butyl-4-(4-chlorophenyl)-2-phenyl-1H-imidazol-5-yl]methyl}-N,N-bis[4- |
| | (difluoromethoxy)benzyl]amine |
| 2709. | 1-(1,3-benzodioxol-5-yl)-N-(1,3-benzodioxol-5-ylmethyl)-N-{[1-butyl-4-(3- |
| | chlorophenyl)-2-phenyl-1H-imidazol-5-yl]methyl}methanamine |
| 2710. | 1-(1,3-benzodioxol-5-yl)-N-(1,3-benzodioxol-5-ylmethyl)-N-{[1-butyl-4-(4- |
| | chlorophenyl)-2-phenyl-1H-imidazol-5-yl]methyl}methanamine |
| 2711. | 1-(1,3-benzodioxol-5-yl)-N-(1,3-benzodioxol-5-ylmethyl)-N-{[1-butyl-4-(3- |
| | fluorophenyl)-2-phenyl-1H-imidazol-5-yl]methyl}methanamine |
| 2712. | 1-(1,3-benzodioxol-5-yl)-N-(1,3-benzodioxol-5-ylmethyl)-N-{[1-butyl-4-(3- |
| | methylphenyl)-2-phenyl-1H-imidazol-5-yl]methyl}methanamine |
| 2713. | 1-(5-{[bis(1,3-benzodioxol-5-ylmethyl)amino]methyl}-1-butyl-2-phenyl-1H-imidazol- |
| | 4-yl)ethanone |
| 2714. | 5-{[bis(1,3-benzodioxol-5-ylmethyl)amino]methyl}-1-butyl-2-phenyl-1H-imidazole-4- |
| | carbaldehyde |
| 2715. | 1-(1,3-benzodioxol-5-yl)-N-(1,3-benzodioxol-5-ylmethyl)-N-{[1-butyl-2-phenyl-4- |
| | (phenylthio)-1H-imidazol-5-yl]methyl}methanamine |
| 2716. | ethyl 5-({(1,3-benzodioxol-5-ylmethyl)[(1-butyl-2,4-diphenyl-1H-imidazol-5- |
| | yl)methyl]amino}methyl)indoline-1-carboxylate |
| 2717. | ethyl 5-({[(1-butyl-2,4-diphenyl-1H-imidazol-5-yl)methyl][4- |
| | (difluoromethoxy)benzyl]amino}methyl)indoline-1-carboxylate |
| 2718. | N-benzyl-1-(1-butyl-2,4-diphenyl-1H-imidazol-5-yl)-N-[2-chloro-3- |
| | (trifluoromethyl)benzyl]methanamine |
| 2719. | N-(2,3-dihydro-1H-inden-2-yl)-N-(2-fluorobenzyl)-2-[8-methoxy-1-(2-methylphenyl)- |
| | 3,4-dihydroisoquinolin-2(1H)-yl]acetamide |
| 2720. | N-(2,3-dihydro-1H-inden-2-yl)-N-(2-fluorobenzyl)-2-[1-methyl-1-(2-methylphenyl)- |
| | 3,4-dihydroisoquinolin-2(1H)-yl]acetamide |
| 2721. | N-(2,3-dihydro-1H-inden-2-yl)-2-[1-ethyl-1-(2-methylphenyl)-3,4-dihydroisoquinolin- |
| | 2(1H)-yl]-N-(2-fluorobenzyl)acetamide |
| 2722. | 2-[1-(2,4-difluorophenyl)-3,4-dihydroisoquinolin-2(1H)-yl]-N-(2,3-dihydro-1H-inden- |
| | 2-yl)-N-(2-fluorobenzyl)acetamide |
| 2723. | N-(2-fluorobenzyl)-N-{3-[1-(2-methylphenyl)-3,4-dihydroisoquinolin-2(1H)-yl]-3- |
| | oxopropyl}indan-2-amine |
| 2724. | N-(2,3-dihydro-1H-inden-2-yl)-N-(2-fluorobenzyl)-2-[1-(2-methylphenyl)-3,4- |
| | dihydroisoquinolin-2(1H)-yl]propanamide |
| 2725. | N-(2,3-dihydro-1H-inden-2-yl)-2-[1-(2-methylphenyl)-3,4-dihydroisoquinolin-2(1H)- |
| | yl]-N-(pyridin-2-ylmethyl)acetamide |
| 2726. | N-(2,3-dihydro-1H-inden-2-yl)-2-[1-(2-methylphenyl)-3,4-dihydroisoquinolin-2(1H)- |
| | yl]-N-(pyridin-3-ylmethyl)acetamide |
| 2727. | 2-[1-(2-bromophenyl)-3,4-dihydroisoquinolin-2(1H)-yl]-N-(2,3-dihydro-1H-inden-2- |
| | yl)-N-(2-fluorobenzyl)acetamide |
| 2728. | N-(2,3-dihydro-1H-inden-2-yl)-2-[1-(2-methylphenyl)-3,4-dihydroisoquinolin-2(1H)- |
| | yl]-N-(1,3-thiazol-2-ylmethyl)acetamide |
| 2729. | N-(2,3-dihydro-1H-inden-2-yl)-N-(2-fluorobenzyl)-2-[1-(2-methoxyphenyl)-3,4- |
| | dihydroisoquinolin-2(1H)-yl]acetamide |
| 2730. | N-(2,3-dihydro-1H-inden-2-yl)-2-[1-(2,3-dimethylphenyl)-3,4-dihydroisoquinolin- |
| | 2(1H)-yl)]-N-(2-fluorobenzyl)acetamide |
| 2731. | N-(2,3-dihydro-1H-inden-2-yl)-N-(2-fluorobenzyl)-2-[(1R)-1-(2-methylphenyl)-3,4- |
| | dihydroisoquinolin-2(1H)-yl]acetamide |
| 2732. | N-(2,3-dihydro-1H-inden-2-yl)-N-(2-fluorobenzyl)-2-[(4R)-4-methyl-1-(2- |
| | methylphenyl)-3,4-dihydroisoquinolin-2(1H)-yl]acetamide |
| 2733. | N-(2,3-dihydro-1H-inden-2-yl)-2-[1-(2-ethylphenyl)-3,4-dihydroisoquinolin-2(1H)-yl]- |
| | N-(2-fluorobenzyl)acetamide |
| 2734. | N-(2-Fluoro-benzyl)-N-indan-2-yl-2- |
| | (4-methyl-1-o-tolyl-3,4-dihydro-1H- |
| | isoquinolin-2-yl)-acetamide |
| 2735. | N-(2,3-dihydro-1H-inden-2-yl)-N-(2-fluorobenzyl)-2-[(1R,4S)-4-methyl-1-(2- |
| | methylphenyl)-3,4-dihydroisoquinolin-2(1H)-yl]acetamide |
| 2736. | N-(2,3-dihydro-1H-inden-2-yl)-N-(2-fluorobenzyl)-2-[(1S,4S)-4-methyl-1-(2- |
| | methylphenyl)-3,4-dihydroisoquinolin-2(1H)-yl]acetamide |
| 2737. | 2-{[1-(2,3-dihydro-1H-inden-2-yl)-2-phenyl-1H-imidazol-5-yl]methyl}-1-(2- |
| | methylphenyl)-1,2,3,4-tetrahydroisoquinoline |
| 2738. | (2S)-N-(2,3-dihydro-1H-inden-2-yl)-N-(2-fluorobenzyl)-2-[(1R)-1-(2-methylphenyl)- |
| | 3,4-dihydroisoquinolin-2(1H)-yl]propanamide |
| 2739. | N-(2,3-dihydro-1H-inden-2-yl)-2-[1-(3,4-dimethylphenyl)-3,4-dihydroisoquinolin- |
| | 2(1H)-yl]-N-(2-fluorobenzyl)acetamide |
| 2740. | 2-[1-(2,3-dichlorophenyl)-3,4-dihydroisoquinolin-2(1H)-yl]-N-(2,3-dihydro-1H-inden- |
| | 2-yl)-N-(2-fluorobenzyl)acetamide |
| 2741. | N-(2,3-dihydro-1H-inden-2-yl)-N-(2-fluorobenzyl)-2-[1-[4-fluoro-2- |
| | (trifluoromethyl)phenyl]-3,4-dihydroisoquinolin-2(1H)-yl]acetamide |
| 2742. | N-(2,3-dihydro-1H-inden-2-yl)-N-(2-fluorobenzyl)-2-[1-[5-fluoro-2- |
| | (trifluoromethyl)phenyl]-3,4-dihydroisoquinolin-2(1H)-yl]acetamide |
| 2743. | (2S)-N-(2,3-dihydro-1H-inden-2-yl)-N-(2-fluorobenzyl)-2-[(1S)-1-(2-methylphenyl)- |
| | 3,4-dihydroisoquinolin-2(1H)-yl]propanamide |
| 2744. | 2-[(1S)-1-(2-bromophenyl)-3,4-dihydroisoquinolin-2(1H)-yl]-N-(2,3-dihydro-1H- |
| | inden-2-yl)-N-(2-fluorobenzyl)acetamide |
| 2745. | N-(2,3-dihydro-1H-inden-2-yl)-N-(2-fluorobenzyl)-2-[(1S)-1-[2- |
| | (trifluoromethyl)phenyl]-3,4-dihydroisoquinolin-2(1H)-yl]acetamide |
| 2746. | 2-[1-(1,1′-biphenyl-2-yl)-3,4-dihydroisoquinolin-2(1H)-yl]-N-(2,3-dihydro-1H-inden-2- |
| | yl)-N-(2-fluorobenzyl)acetamide |
| 2747. | N-(2,3-dihydro-1H-inden-2-yl)-N-(2-fluorobenzyl)-2-[1-(1-naphthyl)-3,4- |
| | dihydroisoquinolin-2(1H)-yl]acetamide |
| 2748. | 2-[1-(2-chloro-3-methylphenyl)-3,4-dihydroisoquinolin-2(1H)-yl]-N-(2,3-dihydro-1H- |
| | inden-2-yl)-N-(2-fluorobenzyl)acetamide |
| 2749. | N-(2,3-dihydro-1H-inden-2-yl)-N-(2-fluorobenzyl)-2-[1-(3-fluoro-2-methylphenyl)-3,4- |
| | dihydroisoquinolin-2(1H)-yl]acetamide |
| 2750. | N-(2,3-dihydro-1H-inden-2-yl)-2-[1-(2,5-dimethylphenyl)-3,4-dihydroisoquinolin- |
| | 2(1H)-yl]-N-(2-fluorobenzyl)acetamide |
| 2751. | N-(2,3-dihydro-1H-inden-2-yl)-N-(2-fluorobenzyl)-2-[1-[3-(trifluoromethyl)phenyl]- |
| | 3,4-dihydroisoquinolin-2(1H)-yl]acetamide |
| 2752. | 2-[1-(5-chloro-2-methylphenyl)-3,4-dihydroisoquinolin-2(1H)-yl]-N-(2,3-dihydro-1H- |
| | inden-2-yl)-N-(2-fluorobenzyl)acetamide |
| 2753. | 2-[1-(2-chloro-5-methylphenyl)-3,4-dihydroisoquinolin-2(1H)-yl]-N-(2,3-dihydro-1H- |
| | inden-2-yl)-N-(2-fluorobenzyl)acetamide |
| 2754. | 2-[1-(2,3-dihydro-1-benzofuran-7-yl)-3,4-dihydroisoquinolin-2(1H)-yl]-N-(2,3- |
| | dihydro-1H-inden-2-yl)-N-(2-fluorobenzyl)acetamide |
| 2755. | N-(2,3-dihydro-1H-inden-2-yl)-N-(2-fluorobenzyl)-2-[(1R,4R)-1-(2-fluorophenyl)-4- |
| | methyl-3,4-dihydroisoquinolin-2(1H)-yl]acetamide |
| 2756. | N-(2,3-dihydro-1H-inden-2-yl)-N-(2-fluorobenzyl)-2-[(3S)-3-methyl-1-(2- |
| | methylphenyl)-3,4-dihydroisoquinolin-2(1H)-yl]acetamide |
| 2757. | N-(2,3-dihydro-1H-inden-2-yl)-2-[1-(2,6-dimethylphenyl)-3,4-dihydroisoquinolin- |
| | 2(1H)-yl]-N-(2-fluorobenzyl)acetamide |
| 2758. | N-(2,3-dihydro-1H-inden-2-yl)-N-(2-fluorobenzyl)-2-[1-(5-fluoro-2-methylphenyl)-3,4- |
| | dihydroisoquinolin-2(1H)-yl]acetamide |
| 2759. | N-(2,3-dihydro-1H-inden-2-yl)-N-(2-fluorobenzyl)-2-[(1R)-1-(2-fluorophenyl)-3,4- |
| | dihydroisoquinolin-2(1H)-yl]acetamide |
| 2760. | 2-[(1R,4R)-1-(2-chlorophenyl)-4-methyl-3,4-dihydroisoquinolin-2(1H)-yl]-N-(2,3- |
| | dihydro-1H-inden-2-yl)-N-(2-fluorobenzyl)acetamide |
| 2761. | (2S)-N-(2,3-dihydro-1H-inden-2-yl)-N-(2-fluorobenzyl)-2-[(1R)-1-(2-fluorophenyl)- |
| | 3,4-dihydroisoquinolin-2(1H)-yl]propanamide |
| 2762. | N-(2,3-dihydro-1H-inden-2-yl)-N-(2-fluorobenzyl)-2-[1-(3-fluoro-4-methylphenyl)-3,4- |
| | dihydroisoquinolin-2(1H)-yl]acetamide |
| 2763. | N-(2,3-dihydro-1H-inden-2-yl)-N-(2-fluorobenzyl)-2-(1-quinolin-8-yl-3,4- |
| | dihydroisoquinolin-2(1H)-yl)acetamide |
| 2764. | (2S)-N-(2,3-dihydro-1H-inden-2-yl)-N-(2-fluorobenzyl)-2-[(1R)-1-(1-naphthyl)-3,4- |
| | dihydroisoquinolin-2(1H)-yl]propanamide |
| 2765. | (2S)-2-[(1R)-1-(2-chlorophenyl)-3,4-dihydroisoquinolin-2(1H)-yl]-N-(2,3-dihydro-1H- |
| | inden-2-yl)-N-(2-fluorobenzyl)propanamide |
| 2766. | 2-[1-(3-chloro-2-methylphenyl)-3,4-dihydroisoquinolin-2(1H)-yl]-N-(2,3-dihydro-1H- |
| | inden-2-yl)-N-(2-fluorobenzyl)acetamide |
| 2767. | N-(2,3-dihydro-1H-inden-2-yl)-N-(2-fluorobenzyl)-2-[1-(2-fluoro-5-methylphenyl)-3,4- |
| | dihydroisoquinolin-2(1H)-yl]acetamide |
| 2768. | (2S)-N-(2,3-dihydro-1H-inden-2-yl)-2-[(1R)-1-(2,3-dimethylphenyl)-3,4- |
| | dihydroisoquinolin-2(1H)-yl]-N-(2-fluorobenzyl)propanamide |
| 2769. | (2S)-N-(2,3-dihydro-1H-inden-2-yl)-2-[(1S)-1-(2,3-dimethylphenyl)-3,4- |
| | dihydroisoquinolin-2(1H)-yl]-N-(2-fluorobenzyl)propanamide |
| 2770. | (2S)-N-(2,3-dihydro-1H-inden-2-yl)-N-(2-fluorobenzyl)-2-[(1R)-1-[2- |
| | (trifluoromethyl)phenyl]-3,4-dihydroisoquinolin-2(1H)-yl]propanamide |
| 2771. | (2S)-2-[(1R)-1-(2-bromophenyl)-3,4-dihydroisoquinolin-2(1H)-yl]-N-(2,3-dihydro-1H- |
| | inden-2-yl)-N-(2-fluorobenzyl)propanamide |
| 2772. | (2S)-N-(2,3-dihydro-1H-inden-2-yl)-2-[(1R)-1-(2,6-dimethylphenyl)-3,4- |
| | dihydroisoquinolin-2(1H)-yl]-N-(2-fluorobenzyl)propanamide |
| 2773. | N-(2,3-dihydro-1H-inden-2-yl)-2-[6,7-dimethyl-1-(2-methylphenyl)-3,4- |
| | dihydroisoquinolin-2(1H)-yl]-N-(2-fluorobenzyl)acetamide |
| 2774. | N-(2,3-dihydro-1H-inden-2-yl)-2-[7,8-dimethyl-1-(2-methylphenyl)-3,4- |
| | dihydroisoquinolin-2(1H)-yl]-N-(2-fluorobenzyl)acetamide |
| 2775. | 2-[1-(2,3-difluorophenyl)-3,4-dihydroisoquinolin-2(1H)-yl]-N-(2,3-dihydro-1H-inden- |
| | 2-yl)-N-(2-fluorobenzyl)acetamide |
| 2776. | (2S)-2-[(1R)-1-(2,3-dihydro-1-benzofuran-7-yl)-3,4-dihydroisoquinolin-2(1H)-yl]-N- |
| | (2,3-dihydro-1H-inden-2-yl)-N-(2-fluorobenzyl)propanamide |
| 2777. | methyl 4-(2-{2-[2,3-dihydro-1H-inden-2-yl(2-fluorobenzyl)amino]-2-oxoethyl}- |
| | 1,2,3,4-tetrahydroisoquinolin-1-yl)benzoate |
| 2778. | (2S)-N-benzyl-2-[(1R)-1-(2-bromophenyl)-3,4-dihydroisoquinolin-2(1H)-yl]-N-(2- |
| | chloro-4-hydroxybenzyl)propanamide |
| 2779. | N-(2,3-dihydro-1H-inden-2-yl)-N-(2-fluorobenzyl)-2-[8-methyl-1-(2-methylphenyl)- |
| | 3,4-dihydroisoquinolin-2(1H)-yl]acetamide |
| 2780. | N-(2,3-dihydro-1H-inden-2-yl)-N-(3-methoxybenzyl)-2-[1-(2-methylphenyl)-3,4- |
| | dihydroisoquinolin-2(1H)-yl]acetamide |
| 2781. | N-(2,3-dihydro-1H-inden-2-yl)-N-(2-fluorobenzyl)-2-(1-mesityl-3,4- |
| | dihydroisoquinolin-2(1H)-yl)acetamide |
| 2782. | 2-[1-(2,6-difluorophenyl)-3,4-dihydroisoquinolin-2(1H)-yl]-N-(2,3-dihydro-1H-inden- |
| | 2-yl)-N-(2-fluorobenzyl)acetamide |
| 2783. | N-(2-fluorobenzyl)-2-[1-(2-methylphenyl)-3,4-dihydroisoquinolin-2(1H)-yl]-N- |
| | phenylacetamide |
| 2784. | N-(2,3-dihydro-1H-inden-2-yl)-N-(2-fluorobenzyl)-2-[1-(2-methyl-1-naphthyl)-3,4- |
| | dihydroisoquinolin-2(1H)-yl]acetamide |
| 2785. | (2S)-2-[(1R)-1-(2-chloro-5-methylphenyl)-3,4-dihydroisoquinolin-2(1H)-yl]-N-(2,3- |
| | dihydro-1H-inden-2-yl)-N-(2-fluorobenzyl)propanamide |
| 2786. | (2S)-N-(2,3-dihydro-1H-inden-2-yl)-2-[(1S)-1-(2,5-dimethylphenyl)-3,4- |
| | dihydroisoquinolin-2(1H)-yl]-N-(2-fluorobenzyl)propanamide |
| 2787. | (2S)-N-(2,3-dihydro-1H-inden-2-yl)-N-(2-fluorobenzyl)-2-[(1R)-1-(2-fluoro-5- |
| | methylphenyl)-3,4-dihydroisoquinolin-2(1H)-yl]propanamide |
| 2788. | (2S)-2-[(1R)-1-(2-chlorophenyl)-3,4-dihydroisoquinolin-2(1H)-yl]-N,N-bis(2- |
| | fluorobenzyl)propanamide |
| 2789. | 4-[(2,3-dihydro-1H-inden-2-yl{[1-(2-methylphenyl)-3,4-dihydroisoquinolin-2(1H)- |
| | yl]acetyl}amino)methyl]benzoic acid |
| 2790. | (2S)-2-[(1R)-1-(2,6-difluorophenyl)-3,4-dihydroisoquinolin-2(1H)-yl]-N-(2,3-dihydro- |
| | 1H-inden-2-yl)-N-(2-fluorobenzyl)propanamide |
| 2791. | (2S)-2-[(1R)-1-(2-chlorophenyl)-3,4-dihydroisoquinolin-2(1H)-yl]-N-(2,3-dihydro-1H- |
| | inden-2-yl)-N-(2-hydroxybenzyl)propanamide |
| 2792. | (2S)-2-[(1R)-1-(2-chlorophenyl)-3,4-dihydroisoquinolin-2(1H)-yl]-N-(2-fluorobenzyl)- |
| | N-(2-phenylethyl)propanamide |
| 2793. | N-(2,3-dihydro-1H-inden-2-yl)-N-(3-hydroxybenzyl)-2-[1-(2-methylphenyl)-3,4- |
| | dihydroisoquinolin-2(1H)-yl]acetamide |
| 2794. | 2-[1-(2-chlorophenyl)-3,4-dihydroisoquinolin-2(1H)-yl]-N-(2-fluorobenzyl)-N-(5- |
| | methoxy-2,3-dihydro-1H-inden-2-yl)acetamide |
| 2795. | N-(2-fluorobenzyl)-N-(5-methoxy-2,3-dihydro-1H-inden-2-yl)-2-[1-[2- |
| | (trifluoromethyl)phenyl]-3,4-dihydroisoquinolin-2(1H)-yl]acetamide |
| 2796. | 2-[1-(2,6-dichlorophenyl)-3,4-dihydroisoquinolin-2(1H)-yl]-N-(2,3-dihydro-1H-inden- |
| | 2-yl)-N-(2-fluorobenzyl)acetamide |
| 2797. | 2-[1-(2-chloro-6-fluorophenyl)-3,4-dihydroisoquinolin-2(1H)-yl]-N-(2,3-dihydro-1H- |
| | inden-2-yl)-N-(2-fluorobenzyl)acetamide |
| 2798. | (2S)-2-[(1R)-1-(2-chlorophenyl)-3,4-dihydroisoquinolin-2(1H)-yl]-N-(2,3-dihydro-1H- |
| | inden-2-yl)-N-(1H-imidazol-4-ylmethyl)propanamide |
| 2799. | 3-{[{(2S)-2-[(1R)-1-(2-chlorophenyl)-3,4-dihydroisoquinolin-2(1H)-yl]propanoyl}(2,3- |
| | dihydro-1H-inden-2-yl)amino]methyl}benzoic acid |
| 2800. | N-(2,3-dihydro-1H-inden-2-yl)-N-(2-fluorobenzyl)-2[1-[2-fluoro-6- |
| | (trifluoromethyl)phenyl]-3,4-dihydroisoquinolin-2(1H)-yl]acetamide |
| 2801. | N-(2,3-dihydro-1H-inden-2-yl)-N-(2-fluorobenzyl)-2-[8-fluoro-1-(2-methylphenyl)-3,4- |
| | dihydroisoquinolin-2(1H)-yl]acetamide |
| 2802. | N-(2,3-dihydro-1H-inden-2-yl)-N-(2-fluoro-5-hydroxybenzyl)-2-[1-(2-methylphenyl)- |
| | 3,4-dihydroisoquinolin-2(1H)-yl]acetamide |
| 2803. | (2S)-2-[(1R)-1-(2,6-dichlorophenyl)-3,4-dihydroisoquinolin-2(1H)-yl]-N-(2,3-dihydro- |
| | 1H-inden-2-yl)-N-(2-fluorobenzyl)propanamide |
| 2804. | (2S)-2-[(1R)-1-(2-chloro-6-fluorophenyl)-3,4-dihydroisoquinolin-2(1H)-yl]-N-(2,3- |
| | dihydro-1H-inden-2-yl)-N-(2-fluorobenzyl)propanamide |
| 2805. | (2S)-2-[(1R)-1-(2-chlorophenyl)-3,4-dihydroisoquinolin-2(1H)-yl]-N-[2-(2- |
| | fluorophenyl)ethyl]-N-(4-hydroxybenzyl)propanamide |
| 2806. | 3-{[{(2S)-2-[(1R)-1-(2-chlorophenyl)-3,4-dihydroisoquinolin-2(1H)-yl]propanoyl}(2,3- |
| | dihydro-1H-inden-2-yl)amino]methyl}-N,N-dimethylbenzamide |
| 2807. | (2S)-2-[(1R)-1-(2-chlorophenyl)-3,4-dihydroisoquinolin-2(1H)-yl]-N-(2,3-dihydro-1H- |
| | inden-2-yl)-N-(4-hydroxy-3,5-dimethylbenzyl)propanamide |
| 2808. | (2S)-2-[(1R)-1-(2-chlorophenyl)-3,4-dihydroisoquinolin-2(1H)-yl]-N-[2-(2- |
| | fluorophenyl)ethyl]-N-(2-phenylethyl)propanamide |
| 2809. | (2S)-2-[(1R)-1-(2-chlorophenyl)-3,4-dihydroisoquinolin-2(1H)-yl]-N-(2-fluorobenzyl)- |
| | N-[2-(2-fluorophenyl)ethyl]propanamide |
| 2810. | (2S)-2-[(1R)-1-(2,6-dichlorophenyl)-3,4-dihydroisoquinolin-2(1H)-yl]-N-(2,3-dihydro- |
| | 1H-inden-2-yl)-N-(4-hydroxy-3,5-dimethylbenzyl)propanamide |
| 2811. | (2S)-2-[(1R)-1-(2-chlorophenyl)-3,4-dihydroisoquinolin-2(1H)-yl]-N-(3-cyanobenzyl)- |
| | N-(2,3-dihydro-1H-inden-2-yl)propanamide |
| 2812. | (2S)-2-[(1R)-1-(2-chlorophenyl)-3,4-dihydroisoquinolin-2(1H)-yl]-N-(2,3-dihydro-1H- |
| | inden-2-yl)-N-(3-nitrobenzyl)propanamide |
| 2813. | N-(2,3-dihydro-1H-inden-2-yl)-N-(3-hydroxybenzyl)-2-[(1S)-1-(2-methylphenyl)-3,4- |
| | dihydroisoquinolin-2(1H)-yl]acetamide |
| 2814. | (2S)-2-[(1R)-1-(2-chlorophenyl)-3,4-dihydroisoquinolin-2(1H)-yl]-N-(2,3-dihydro-1H- |
| | inden-2-yl)-N-(3-hydroxybenzyl)propanamide |
| 2815. | (2S)-2-[(1R)-1-(2-chlorophenyl)-3,4-dihydroisoquinolin-2(1H)-yl]-N-(2,3-dihydro-1H- |
| | inden-2-yl)-N-(3-fluoro-3-hydroxybenzyl)propanamide |
| 2816. | 2-[8-chloro-1-(2-methylphenyl)-3,4-dihydroisoquinolin-2(1H)-yl]-N-(2,3-dihydro-1H- |
| | inden-2-yl)-N-(2-fluorobenzyl)acetamide |
| 2817. | 2-[8-chloro-1-(2-chloro-6-fluoropheneyl)-3,4-dihydroisoquinolin-2(1H)-yl]-N-(2,3- |
| | dihydro-1H-inden-2-yl)-N-(2-fluorobenzyl)acetamide |
| 2818. | (2S)-N-(2-fluorobenzyl)-N-[2-(4-hydroxyphenyl)ethyl]-2-[(1R)-1-(1-naphthyl)-3,4- |
| | dihydroisoquinolin-2(1H)-yl]propanamide |
| 2819. | (2S)-N-(2-fluorobenzyl)-N-[2-(1H-indol-3-yl)ethyl]-2-[(1R)-1-(1-naphthyl)-3,4- |
| | dihydroisoquinolin-2(1H)-yl]propanamide |
| 2820. | (2S)-2-[(1R)-1-(2-chlorophenyl)-3,4-dihydroisoquinolin-2(1H)-yl]-N-[3- |
| | (difluoromethoxy)-2-fluorobenzyl]-N-(2,3-dihydro-1H-inden-2-yl)propanamide |
| 2821. | (2S)-N-(2,3-dihydro-1H-inden-2-yl)-N-[(2-methoxypyridin-3-yl)methyl]-2[(1R)-1-(1- |
| | naphthyl)-3,4-dihydroisoquinolin-2(1H)-yl]propanamide |
| 2822. | (2S)-N-(2-fluorobenzyl)-N-[(2-methoxypyridin-3-yl)methyl]-2-[(1R)-1-(1-naphthyl)- |
| | 3,4-dihydroisoquinolin-2(1H)-yl]propanamide |
| 2823. | N-(2,3-dihydro-1H-inden-2-yl)-N-(2-fluorobenzyl)-2-[1-(4-hydroxyphenyl)-3,4- |
| | dihydroisoquinolin-2(1H)-yl]acetamide |
| 2824. | (2S)-N-(2,3-dihydro-1H-inden-2-yl)-N-[(6-methoxypyridin-2-yl)methyl]-2-[(1R)-1-(1- |
| | naphthyl)-3,4-dihydroisoquinolin-2(1H)-yl]propanamide |
| 2825. | (2S)-N-(2-fluorobenzyl)-N-[(6-methoxypyridin-2-yl)methyl]-2-[(1R)-1-(1-naphthyl)- |
| | 3,4-dihydroisoquinolin-2(1H)-yl]propanamide |
| 2826. | (2S)-N-(2,3-dihydro-1H-inden-2-yl)-N-[(3-fluoropyridin-2-yl)methyl]-2-[(1R)-1-(1- |
| | naphthyl)-3,4-dihydroisoquinolin-2(1H)-yl]propanamide |
| 2827. | (2S)-N-(2,3-dihydro-1H-inden-2-yl)-N-[(5-methoxypyridin-3-yl)methyl]-2-(1R)-1-(1- |
| | naphthyl)-3,4-dihydroisoquinolin-2(1H)-yl]propanamide |
| 2828. | 7-({butyl[(1-butyl-4-chloro-2-phenyl-1H-imidazol-5- |
| | yl)methyl]amino}methyl)quinazolin-4-ol |
| 2829. | methyl 4-{[{[1-butyl-4-chloro-2-(2-methylphenyl)-1H-imidazol-5- |
| | yl]methyl}(neopentyl)amino]methyl}-2-methoxybenzoate |
| 2830. | 4-{[{[1-butyl-4-chloro-2-(2-methylphenyl)-1H-imidazol-5- |
| | yl]methyl}(neopentyl)amino]methyl}-2-methoxybenzoic acid |
| 2831. | 4-{[{[1-butyl-4-chloro-2-(2-methylphenyl)-1H-imidazol-5- |
| | yl]methyl}(neopentyl)amino]methyl}-2-methoxybenzamide |
| 2832. | 4-{[{[1-butyl-4-chloro-2-(2-methylphenyl)-1H-imidazol-5- |
| | yl]methyl}(neopentyl)amino]methyl}-2-hydroxybenzamide |
| 2833. | 4-{[{[1-butyl-4-(4-methoxyphenyl)-2-phenyl-1H-imidazol-5- |
| | yl]methyl}(neopentyl)amino]methyl}-2-hydroxybenzamide |
| 2834. | 4-[(benzyl{[1-butyl-4-(4-methoxyphenyl)-2-phenyl-1H-imidazol-5- |
| | yl]methyl}amino)methyl]-2-hydroxybenzamide |
| 2835. | 5-{[bis(2,3-dihydro-1,4-benzodioxin-6-ylmethyl)amino]methyl}-1-butyl-2-phenyl-1H- |
| | imidazole-4-carbonitrile |
| 2836. | N-[(1-butyl-4-fluoro-2-phenyl-1H-imidazol-5-yl)methyl]-N-(2,3-dihydro-1,4- |
| | benzodioxin-6-ylmethyl)-N-(3-ethoxybenzyl)amine |
| 2837. | N-[(1-butyl-4-fluoro-2-phenyl-1H-imidazol-5-yl)methyl]-N,N-bis(2,3-dihydro-1,4- |
| | benzodioxin-6-ylmethyl)amine |
| 2838. | 4-({butyl[(1-butyl-4-chloro-2-phenyl-1H-imidazol-5- |
| | yl)methyl]amino}methyl)benzenesulfonamide |
| 2839. | 4-({butyl[(1-butyl-4-chloro-2-phenyl-1H-imidazol-5- |
| | yl)methyl]amino}methyl)benzamide |
| 2840. | methyl 2-amino-4-({butyl[(1-butyl-2,4-diphenyl-1H-imidazol-5- |
| | yl)methyl]amino}methyl)benzoate |
| 2841. | 7-({butyl[(1-butyl-2,4-diphenyl-1H-imidazol-5-yl)methyl]amino}methyl)quinazolin-4- |
| | ol |
| 2842. | N-(1,3-benzodioxol-5-ylmethyl)-N-[(1-butyl-2,4-diphenyl-1H-imidazol-5-yl)methyl]-4- |
| | isopropylaniline |
| 2843. | 1-[1-butyl-2-(2-methoxyphenyl)-4-(trifluoromethyl)-1H-imidazol-5-yl]-N-(2,3-dihydro- |
| | 1,4-benzodioxin-6-ylmethyl)-N-(3-ethoxybenzyl)methanamine |
| 2844. | 4-({(1,3-benzodioxol-5-ylmethyl)[(1-butyl-2,4-diphenyl-1H-imidazol-5- |
| | yl)methyl]amino}methyl)-N,N-dimethylcyclohexanamine |
| 2845. | 1-(1-butyl-2,4-diphenyl-1H-imidazol-5-yl)-N-(2,3-dihydro-1,4-benzodioxin-6- |
| | ylmethyl)-N-(tetrahydro-2H-pyran-4-ylmethyl)methanamine |
| 2846. | N-{[1-butyl-2-(2-methylphenyl)-4-(trifluoromethyl)-1H-imidazol-5-yl]methyl}-N-(2,3- |
| | dihydro-1,4-benzodioxin-6-ylmethyl)-N-(3-ethoxybenzyl)amine |
| 2847. | N-[1-(1-butyl-4-chloro-2-phenyl-1H-imidazol-5-yl)ethyl]-N-(2,3-dihydro-1,4- |
| | benzodioxin-6-ylmethyl)-N-(3-ethoxybenzyl)amine |
| 2848. | 1-(1,3-benzodioxol-5-yl)-N-(1,3-benzodioxol-5-ylmethyl)-N-{[1-butyl-4-chloro-2-(2- |
| | methoxyphenyl)-1H-imidazol-5-yl]methyl}methanamine |
| 2849. | N-[(1-butyl-4-chloro-2-phenyl-1H-imidazol-5-yl)methyl]-N-(2,3-dihydro-1,4- |
| | benzodioxin-6-ylmethyl)-1-(3-ethoxyphenyl)ethanamine |
| 2850. | methyl 4-{[[(1-butyl-4-chloro-2-phenyl-1H-imidazol-5-yl)methyl](2,3-dihydro-1,4- |
| | benzodioxin-6-ylmethyl)amino]methyl}cyclohexanecarboxylate |
| 2851. | 4-{[[(1-butyl-4-chloro-2-phenyl-1H-imidazol-5-yl)methyl](2,3-dihydro-1,4- |
| | benzodioxin-6-ylmethyl)amino]methyl}cyclohexanecarboxylic acid |
| 2852. | N-[(1-butyl-4-chloro-2-phenyl-1H-imidazol-5-yl)methyl]-1-(2,3-dihydro-1,4- |
| | benzodioxin-6-yl)-N-(3-ethoxybenzyl)ethanamine |
| 2853. | N-[(1-butyl-4-chloro-2-phenyl-1H-imidazol-5-yl)methyl]-N-(cyclopropylmethyl)-N- |
| | (2,3-dihydro-1,4-benzodioxin-6-ylmethyl)amine |
| 2854. | N-[(1-butyl-4-chloro-2-phenyl-1H-imidazol-5-yl)methyl]-N-(2,3-dihydro-1,4- |
| | benzodioxin-6-ylmethyl)-N-[(6-methoxypyridin-3-yl)methyl]amine |
| 2855. | 1-[1-butyl-4-chloro-2-(2-methoxyphenyl)-1H-imidazol-5-yl]-N-(2,3-dihydro-1,4- |
| | benzodioxin-6-ylmethyl)-N-(3-ethoxybenzyl)methanamine |
| 2856. | 5-{[[(1-butyl-4-chloro-2-phenyl-1H-imidazol-5-yl)methyl](2,3-dihydro-1,4- |
| | benzodioxin-6-ylmethyl)amino]methyl}pyridin-2-ol |
| 2857. | N-[(1-butyl-4-chloro-2-phenyl-1H-imidazol-5-yl)methyl]-N-(2,3-dihydro-1,4- |
| | benzodioxin-6-ylmethyl)-N-(3-propoxybenzyl)amine |
| 2858. | N-[(1-butyl-4-chloro-2-phenyl-1H-imidazol-5-yl)methyl]-N-(2,3-dihydro-1,4- |
| | benzodioxin-6-ylmethyl)-N-(3-isopropoxybenzyl)amine |
| 2859. | N-[(1-butyl-4-chloro-2-phenyl-1H-imidazol-5-yl)methyl]-N-(2,3-dihydro-1,4- |
| | benzodioxin-6-ylmethyl)-N-(1H-indazol-6-ylmethyl)amine |
| 2860. | methyl 4-({[(1-butyl-4-chloro-2-phenyl-1H-imidazol-5-yl)methyl][(2-methyl-1,3- |
| | benzothiazol-5-yl)methyl]amino}methyl)benzoate |
| 2861. | 4-{[[(1-butyl-4-chloro-2-phenyl-1H-imidazol-5-yl)methyl](2,3-dihydro-1,4- |
| | benzodioxin-6-ylmethyl)amino]methyl}benzoic acid |
| 2862. | N-[(1-butyl-4-chloro-2-phenyl-1H-imidazol-5-yl)methyl]-N-(2,3-dihydro-1,4- |
| | benzodioxin-6-ylmethyl)-N-[(3,5-dimethyl-1H-pyrazol-4-yl)methyl]amine |
| 2863. | N-[(1-butyl-4-chloro-2-phenyl-1H-imidazol-5-yl)methyl]-N-(2,3-dihydro-1,4- |
| | benzodioxin-6-ylmethyl)-N-[(6-methoxypyridin-2-yl)methyl]amine |
| 2864. | 4-({[(1-butyl-4-chloro-2-phenyl-1H-imidazol-5-yl)methyl][(2-methyl-1,3-benzothiazol- |
| | 5-yl)methyl]amino}methyl)benzoic acid |
| 2865. | 3-[[(1-butyl-4-chloro-2-phenyl-1H-imidazol-5-yl)methyl](2,3-dihydro-1,4-benzodioxin- |
| | 6-ylmethyl)amino]propan-1-ol |
| 2866. | N-[(1-butyl-4-chloro-2-phenyl-1H-imidazol-5-yl)methyl]-N-(2,3-dihydro-1,4- |
| | benzodioxin-6-ylmethyl)-N-(1H-indazol-5-ylmethyl)amine |
| 2867. | N-[(1-butyl-4-chloro-2-phenyl-1H-imidazol-5-yl)methyl]-N-(2,3-dihydro-1,4- |
| | benzodioxin-6-ylmethyl)-3-morpholin-4-ylpropan-1-amine |
| 2868. | 4-{1-[[(1-butyl-4-chloro-2-phenyl-1H-imidazol-5-yl)methyl](2,3-dihydro-1,4- |
| | benzodioxin-6-ylmethyl)amino]ethyl}benzoic acid |
| 2869. | N-[(1-butyl-4-chloro-2-phenyl-1H-imidazol-5-yl)methyl]-N-(2,3-dihydro-1,4- |
| | benzodioxin-6-ylmethyl)-N-[(5-methoxypyridin-3-yl)methyl]amine |
| 2870. | N-[(1-butyl-4-chloro-2-phenyl-1H-imidazol-5-yl)methyl]-N-(2,3-dihydro-1,4- |
| | benzodioxin-6-ylmethyl)-3-(3-methyl-1H-pyrazol-1-yl)propan-1-amine |
| 2871. | N-[(1-butyl-4-chloro-2-phenyl-1H-imidazol-5-yl)methyl]-2-cyclopropyl-N-(2,3- |
| | dihydro-1,4-benzodioxin-6-ylmethyl)ethanamine |
| 2872. | N-[(4-chloro-2-phenyl-1-propyl-1H-imidazol-5-yl)methyl]-1-(2,3-dihydro-1,4- |
| | benzodioxin-6-yl)-N-(3-ethoxybenzyl)ethanamine |
| 2873. | 1-butyl-5-{[(2,3-dihydro-1,4-benzodioxin-6-ylmethyl)(3-ethoxybenzyl)amino]methyl}- |
| | 2-(2-methoxyphenyl)-1H-imidazole-4-carbonitrile |
| 2874. | N-{[4-chloro-1-methyl-2-(2-methylphenyl)-1H-imidazol-5-yl]methyl}-1-(2,3-dihydro- |
| | 1,4-benzodioxin-6-yl)-N-(3-ethoxybenzyl)ethanamine |
| 2875. | N-(1,3-benzodioxol-5-ylmethyl)-N-[(1-methyl-2,4-diphenyl-1H-imidazol-5- |
| | yl)methyl]butan-1-amine |
| 2876. | 1-(1,3-benzodioxol-5-yl)-N-(1,3-benzodioxol-5-ylmethyl)-N-[(1 -methyl-2,4-diphenyl- |
| | 1H-imidazol-5-yl)methyl]methanamine |
| 2877. | N-(1,3-benzodioxol-5-ylmethyl)-N-[(1-ethyl-2,4-diphenyl-1H-imidazol-5- |
| | yl)methyl]butan-1-amine |
| 2878. | 1-(1,3-benzodioxol-5-yl)-N-(1,3 -benzodioxol-5-ylmethyl)-N-[(1-ethyl-2,4-diphenyl- |
| | 1H-imidazol-5-yl)methyl]methanamine |
| 2879. | N-(1,3-benzodioxol-5-ylmethyl)-N-[(2,4-diphenyl-1-propyl-1H-imidazol-5- |
| | yl)methyl]butan-1-amine |
| 2880. | 1-(1,3-benzodioxol-5-yl)-N-(1,3-benzodioxol-5-ylmethyl)-N-[(2,4-diphenyl-1-propyl- |
| | 1H-imidazol-5-yl)methyl]methanamine |
| 2881. | 1-butyl-5-{[(2,3-dihydro-1,4-benzodioxin-6-ylmethyl)(3-ethoxybenzyl)amino]methyl}- |
| | 2-phenyl-1H-imidazole-4-carbonitrile |
| 2882. | 1-(1,3-benzodioxol-5-yl)-N-(1,3-benzodioxol-5-ylmethyl)-N-[(1-butyl-2-cyclohex-1- |
| | en-1-yl-4-phenyl-1H-imidazol-5-yl)methyl]methanamine |
| 2883. | 1-[1-butyl-4-chloro-2-(2-methylphenyl)-1H-imidazol-5-yl]-N-(2,3-dihydro-1,4- |
| | benzodioxin-6-ylmethyl)-N-(3-ethoxybenzyl)methanamine |
| 2884. | 1-(1,3-benzodioxol-5-yl)-N-(1,3-benzodioxol-5-ylmethyl)-N-{[1-butyl-4-chloro-2-(2- |
| | methylphenyl)-1H-imidazol-5-yl]methyl}methanamine |
| 2885. | 1-(4-chloro-2-phenyl-1-propyl-1H-imidazol-5-yl)-N-(2,3-dihydro-1,4-benzodioxin-6- |
| | ylmethyl)-N-(3-ethoxybenzyl)methanamine |
| 2886. | 1-(4-chloro-2-phenyl-1-propyl-1H-imidazol-5-yl)-N,N-bis(2,3-dihydro-1,4- |
| | benzodioxin-6-ylmethyl)methanamine |
| 2887. | 1-(1,3-benzodioxol-5-yl)-N-[(4-chloro-2-phenyl-1-propyl-1H-imidazol-5-yl)methyl]-N- |
| | (3-ethoxybenzyl)methanamine |
| 2888. | 1-(1,3-benzodioxol-5-yl)-N-(1,3-benzodioxol-5-ylmethyl)-N-[(4-chloro-2-phenyl-1- |
| | propyl-1H-imidazol-5-yl)methyl]methanamine |
| 2889. | 1-(2,3-dihydro-1,4-benzodioxin-6-yl)-N-(3-ethoxybenzyl)-N-[(1-methyl-2,4-diphenyl- |
| | 1H-imidazol-5-yl)methyl]methanamine |
| 2890. | 1-(2,3-dihydro-1,4-benzodioxin-6-yl)-N-(2,3-dihydro-1,4-benzodioxin-6-ylmethyl)-N- |
| | [(1-methyl-2,4-diphenyl-1H-imidazol-5-yl)methyl]methanamine |
| 2891. | 1-(1,3-benzodioxol-5-yl)-N-(3-ethoxybenzyl)-N-[(1-methyl-2,4-diphenyl-1H-imidazol- |
| | 5-yl)methyl]methanamine |
| 2892. | 1-(2,3-dihydro-1,4-benzodioxin-6-yl)-N-[(2,4-diphenyl-1-propyl-1H-imidazol-5- |
| | yl)methyl]-N-(3-ethoxybenzyl)methanamine |
| 2893. | 1-(2,3-dihydro-1,4-benzodioxin-6-yl)-N-(2,3-dihydro-1,4-benzodioxin-6-ylmethyl)-N- |
| | [(2,4-diphenyl-1-propyl-1H-imidazol-5-yl)methyl]methanamine |
| 2894. | 1-(1,3-benzodioxol-5-yl)-N-[(2,4-diphenyl-1-propyl-1H-imidazol-5-yl)methyl]-N-(3- |
| | ethoxybenzyl)methanamine |
| 2895. | 1-(1,3-benzodioxol-5-yl)-N-{[1-butyl-4-fluoro-2-(2-methylphenyl)-1H-imidazol-5- |
| | yl]methyl}-N-(3-ethoxybenzyl)methanamine |
| 2896. | 1-[1-butyl-4-fluoro-2-(2-methylphenyl)-1H-imidazol-5-yl]-N-(2,3-dihydro-1,4- |
| | benzodioxin-6-ylmethyl)-N-(3-ethoxybenzyl)methanamine |
| 2897. | 1-(1,3-benzodioxol-5-yl)-N-(1,3-benzodioxol-5-ylmethyl)-N-{[1-butyl-2-(2- |
| | chlorophenyl)-4-phenyl-1H-imidazol-5-yl]methyl}methanamine |
| 2898. | N-{[1-butyl-2-(2-chlorophenyl)-4-phenyl-1H-imidazol-5-yl]methyl}-N-(2,3-dihydro- |
| | 1,4-benzodioxin-6-ylmethyl)-N-(3-ethoxybenzyl)amine |
| 2899. | 5-{[(1,3-benzodioxol-5-ylmethyl)(3-ethoxybenzyl)amino]methyl}-1-butyl-2-(2- |
| | methylphenyl)-1H-imidazole-4-carbonitrile |
| 2900. | 1-butyl-5-{[(2,3-dihydro-1,4-benzodioxin-6-ylmethyl)(3-ethoxybenzyl)amino]methyl}- |
| | 2-(2-methylphenyl)-1H-imidazole-4-carbonitrile |
| 2901. | N-[(1-butyl-4-chloro-2-phenyl-1H-imidazol-5-yl)methyl]-N-(2,3-dihydro-1,4- |
| | benzodioxin-6-ylmethyl)-N-(2-fluoro-5-methoxybenzyl)amine |
| 2902. | N-[(1-butyl-4-chloro-2-phenyl-1H-imidazol-5-yl)methyl]-N-(2,3-dihydro-1,4- |
| | benzodioxin-6-ylmethyl)-N-(4-fluoro-3-methoxybenzyl)amine |
| 2903. | N-[(1-butyl-4-chloro-2-phenyl-1H-imidazol-5-yl)methyl]-N-(2,6-difluorobenzyl)-N- |
| | (2,3-dihydro-1,4-benzodioxin-6-ylmethyl)amine |
| 2904. | N-[(1-butyl-4-chloro-2-phenyl-1H-imidazol-5-yl)methyl]-N-(2,3-dihydro-1,4- |
| | benzodioxin-6-ylmethyl)-N-(3-fluorobenzyl)amine |
| 2905. | N-[(1-butyl-4-chloro-2-phenyl-1H-imidazol-5-yl)methyl]-N-(3-chlorobenzyl)-N-(2,3- |
| | dihydro-1,4-benzodioxin-6-ylmethyl)amine |
| 2906. | N-[(1-butyl-4-chloro-2-phenyl-1H-imidazol-5-yl)methyl]-N-(2,3-dihydro-1,4- |
| | benzodioxin-6-ylmethyl)-N-(3-methylbenzyl)amine |
| 2907. | 1-(3-{[[(1-butyl-4-chloro-2-phenyl-1H-imidazol-5-yl)methyl](2,3-dihydro-1,4- |
| | benzodioxin-6-ylmethyl)amino]methyl}phenyl)ethanone |
| 2908. | N-[(1-butyl-4-chloro-2-phenyl-1H-imidazol-5-yl)methyl]-N-(2,3-dihydro-1,4- |
| | benzodioxin-6-ylmethyl)-N-(3-methoxybenzyl)amine |
| 2909. | 3-{[[(1-butyl-4-chloro-2-phenyl-1H-imidazol-5-yl)methyl](2,3-dihydro-1,4- |
| | benzodioxin-6-ylmethyl)amino]methyl}benzoic acid |
| 2910. | 3-{[[(1-butyl-4-chloro-2-phenyl-1H-imidazol-5-yl)methyl](2,3-dihydro-1,4- |
| | benzodioxin-6-ylmethyl)amino]methyl}benzamide |
| 2911. | 3-{[[(1-butyl-4-chloro-2-phenyl-1H-imidazol-5-yl)methyl](2,3-dihydro-1,4- |
| | benzodioxin-6-ylmethyl)amino]methyl}benzonitrile |
| 2912. | N-[(1-butyl-2,4-diphenyl-1H-imidazol-5-yl)methyl]-N-(2,3-dihydro-1,4-benzodioxin-6- |
| | ylmethyl)-2-methoxyethanamine |
| 2913. | N-[(1-butyl-2,4-diphenyl-1H-imidazol-5-yl)methyl]-N-(2,3-dihydro-1,4-benzodioxin-6- |
| | ylmethyl)-3-methoxypropan-1-amine |
| 2914. | N-[(1-butyl-2,4-diphenyl-1H-imidazol-5-yl)methyl]-N-(2,3-dihydro-1,4-benzodioxin-6- |
| | ylmethyl)-2-ethoxyethanamine |
| 2915. | 4-[[(1-butyl-2,4-diphenyl-1H-imidazol-5-yl)methyl](2,3-dihydro-1,4-benzodioxin-6- |
| | ylmethyl)amino]butan-1-ol |
| 2916. | N-[(1-butyl-4-chloro-2-phenyl-1H-imidazol-5-yl)methyl]-N-(2,3-dihydro-1,4- |
| | benzodioxin-6-ylmethyl)-2-methoxyethanamine |
| 2917. | N-[(1-butyl-4-chloro-2-phenyl-1H-imidazol-5-yl)methyl]-N-(2,3-dihydro-1,4- |
| | benzodioxin-6-ylmethyl)-3-methoxypropan-1-amine |
| 2918. | N-[(1-butyl-4-chloro-2-phenyl-1H-imidazol-5-yl)methyl]-N-(2,3-dihydro-1,4- |
| | benzodioxin-6-ylmethyl)-2-ethoxyethanamine |
| 2919. | 4-[[(1-butyl-4-chloro-2-phenyl-1H-imidazol-5-yl)methyl](2,3-dihydro-1,4-benzodioxin- |
| | 6-ylmethyl)amino]butan-1-ol |
| 2920. | 1-[1-butyl-2-(2-methyl-1,3-thiazol-4-yl)-4-phenyl-1H-imidazol-5-yl]-N-(2,3-dihydro- |
| | 1,4-benzodioxin-6-ylmethyl)-N-(3-ethoxybenzyl)methanamine |
| 2921. | 1-[4-chloro-1-methyl-2-(2-methylphenyl)-1H-imidazol-5-yl]-N-(2,3-dihydro-1,4- |
| | benzodioxin-6-ylmethyl)-N-(3-ethoxybenzyl)methanamine |
| 2922. | 1-[4-chloro-1-methyl-2-(2-methylphenyl)-1H-imidazol-5-yl]-N-(3-ethoxybenzyl)-N- |
| | (1H-indol-5-ylmethyl)methanamine |
| 2923. | 1-[1-butyl-4-chloro-2-(4-fluorophenyl)-1H-imidazol-5-yl]-N-(2,3-dihydro-1,4- |
| | benzodioxin-6-ylmethyl)-N-(3-ethoxybenzyl)methanamine |
| 2924. | 1-(4-{[[(1-butyl-4-chloro-2-phenyl-1H-imidazol-5-yl)methyl](2,3-dihydro-1,4- |
| | benzodioxin-6-ylmethyl)amino]methyl}phenyl)ethanone |
| 2925. | N-[(1-butyl-4-chloro-2-phenyl-1H-imidazol-5-yl)methyl]-N-(2,3-dihydro-1,4- |
| | benzodioxin-6-ylmethyl)-N-(4-methylbenzyl)amine |
| 2926. | N-[(4-bromo-1-butyl-2-phenyl-1H-imidazol-5-yl)methyl]-N-(2,3-dihydro-1,4- |
| | benzodioxin-6-ylmethyl)-N-(3-ethoxybenzyl)amine |
| 2927. | N-[(1-butyl-4-methyl-2-phenyl-1H-imidazol-5-yl)methyl]-N-(2,3-dihydro-1,4- |
| | benzodioxin-6-ylmethyl)-N-(3-ethoxybenzyl)amine |
| 2928. | 1-[1-butyl-4-fluoro-2-(2-methylphenyl)-1H-imidazol-5-yl]-N-(3-ethoxybenzyl)-N-(1H- |
| | indol-5-ylmethyl)methanamine |
| 2929. | N-[(1-butyl-4-chloro-2-phenyl-1H-imidazol-5-yl)methyl]-N-(2,3-dihydro-1,4- |
| | benzodioxin-6-ylmethyl)-N-(1H-indol-5-ylmethyl)amine |
| 2930. | 1-(2,3-dihydro-1,4-benzodioxin-6-yl)-N-(3-ethoxybenzyl)-N-[(4-methyl-2-phenyl-1- |
| | propyl-1H-imidazol-5-yl)methyl]methanamine |
| 2931. | 6-{[[(1-butyl-4-chloro-2-phenyl-1H-imidazol-5-yl)methyl](2,3-dihydro-1,4- |
| | benzodioxin-6-ylmethyl)amino]methyl}-1,2-benzisoxazol-3-amine |
| 2932. | 2-[[(1-butyl-4-chloro-2-phenyl-1H-imidazol-5-yl)methyl](2,3-dihydro-1,4-benzodioxin- |
| | 6-ylmethyl)amino]-1-(3-methoxyphenyl)ethanone |
| 2933. | 2-[Butyl-(3-butyl-5-chloro-2-phenyl-3H-imidazol-4-ylmethyl)-amino]-N-(2,3-dihydro- |
| | benzo[1,4]dioxin-6-ylmethyl)-acetamide |
| 2934. | 2-[[(1-butyl-4-chloro-2-phenyl-1H-imidazol-5-yl)methyl](2,3-dihydro-1,4-benzodioxin- |
| | 6-ylmethyl)amino]-1-(3-methoxyphenyl)ethanol |
| 2935. | N-[2-(benzyloxy)ethyl]-N-[(1-butyl-4-chloro-2-phenyl-1H-imidazol-5-yl)methyl]butan- |
| | 1-amine |
| 2936. | N-[2-(benzyloxy)ethyl]-N-[(1-butyl-4-chloro-2-phenyl-1H-imidazol-5-yl)methyl]-N- |
| | (2,3-dihydro-1,4-benzodioxin-6-ylmethyl)amine |
| 2937. | methyl 3-{[[(1-butyl-4-chloro-2-phenyl-1H-imidazol-5-yl)methyl](2,3-dihydro-1,4- |
| | benzodioxin-6-ylmethyl)amino]methyl}benzoate |
| 2938. | 5-{[[(1-butyl-4-chloro-2-phenyl-1H-imidazol-5-yl)methyl](2,3-dihydro-1,4- |
| | benzodioxin-6-ylmethyl)amino]methyl}-N-(2-methoxyethyl)pyridin-2-amine |
| 2939. | N-[(1-butyl-4-chloro-2-phenyl-1H-imidazol-5-yl)methyl]-N-(2,3-dihydro-1,4- |
| | benzodioxin-6-ylmethyl)-N-[(6-morpholin-4-ylpyridin-3-yl)methyl]amine |
| 2940. | N-[(1-butyl-4-chloro-2-phenyl-1H-imidazol-5-yl)methyl]-N-[(6-chloropyridin-3- |
| | yl)methyl]-N-(2,3-dihydro-1,4-benzodioxin-6-ylmethyl)amine |
| 2941. | N-[(1-butyl-4-chloro-2-phenyl-1H-imidazol-5-yl)methyl]-2-(2,3-dihydro-1,4- |
| | benzodioxin-6-yl)-N-(3-ethoxybenzyl)propan-2-amine |
| 2942. | 4-{[[(1-butyl-4-chloro-2-phenyl-1H-imidazol-5-yl)methyl](2,3-dihydro-1,4- |
| | benzodioxin-6-ylmethyl)amino]methyl}-3-chlorobenzoic acid |
| 2943. | 4-({[(1-butyl-4-chloro-2-phenyl-1H-imidazol-5-yl)methyl][1-(2,3-dihydro-1,4- |
| | benzodioxin-6-yl)ethyl]amino}methyl)benzoic acid |
| 2944. | 4-({butyl[(1-butyl-4-chloro-2-phenyl-1H-imidazol-5-yl)methyl]amino}methyl)-3- |
| | chlorobenzoic acid |
| 2945. | N-[(1-butyl-2,4-diphenyl-1H-imidazol-5-yl)methyl]-N-methyl-1,2,3,4- |
| | tetrahydronaphthalen-1-amine |
| 2946. | 4-({[(1-butyl-4-chloro-2-phenyl-1H-imidazol-5-yl)methyl][4- |
| | (difluoromethoxy)benzyl]amino}methyl)benzoic acid |
| 2947. | N-[(1-butyl-4-chloro-2-phenyl-1H-imidazol-5-yl)methyl]-N-(2,3-dihydro-1,4- |
| | benzodioxin-6-ylmethyl)-N-(1H-pyrazol-4-ylmethyl)amine |
| 2948. | N-[(1-butyl-4-chloro-2-phenyl-1H-imidazol-5-yl)methyl]-N-(2,3-dihydro-1,4- |
| | benzodioxin-6-ylmethyl)-N-[(1-ethyl-1H-pyrazol-4-yl)methyl]amine |
| 2949. | N-[(1-butyl-4-chloro-2-phenyl-1H-imidazol-5-yl)methyl]-N-(2,3-dihydro-1,4- |
| | benzodioxin-6-ylmethyl)-N-(1H-pyrazol-5-ylmethyl)amine |
| 2950. | N-[(1-butyl-4-chloro-2-phenyl-1H-imidazol-5-yl)methyl]-N-(2,3-dihydro-1,4- |
| | benzodioxin-6-ylmethyl)-N-[(1,4-dimethyl-1H-pyrazol-3-yl)methyl]amine |
| 2951. | N-[(1-butyl-4-chloro-2-phenyl-1H-imidazol-5-yl)methyl]-N-(2,3-dihydro-1,4- |
| | benzodioxin-6-ylmethyl)-N-(pyridin-4-ylmethyl)amine |
| 2952. | N-[(1-butyl-4-chloro-2-phenyl-1H-imidazol-5-yl)methyl]-N-(2,3-dihydro-1,4- |
| | benzodioxin-6-ylmethyl)-N-[4-(1H-tetraazol-5-yl)benzyl]amine |
| 2953. | N-[(1-butyl-4-chloro-2-phenyl-1H-imidazol-5-yl)methyl]-N-(2,3-dihydro-1,4- |
| | benzodioxin-6-ylmethyl)-N-[(1-oxidopyridin-4-yl)methyl]amine |
| 2954. | N-[(1-butyl-4-chloro-2-phenyl-1H-imidazol-5-yl)methyl]-N-[(5-chloro-1,3-dimethyl- |
| | 1H-pyrazol-4-yl)methyl]-N-(2,3-dihydro-1,4-benzodioxin-6-ylmethyl)amine |
| 2955. | N-[(1-butyl-4-chloro-2-phenyl-1H-imidazol-5-yl)methyl]-N-{[5-chloro-1-methyl-3- |
| | (trifluoromethyl)-1H-pyrazol-4-yl]methyl}-N-(2,3-dihydro-1,4-benzodioxin-6- |
| | ylmethyl)amine |
| 2956. | 4-{[[(1-butyl-4-chloro-2-phenyl-1H-imidazol-5-yl)methyl](2,3-dihydro-1,4- |
| | benzodioxin-6-ylmethyl)amino]methyl}-3-fluorobenzoic acid |
| 2957. | 5-{3-[[(1-butyl-4-chloro-2-phenyl-1H-imidazol-5-yl)methyl](2,3-dihydro-1,4- |
| | benzodioxin-6-ylmethyl)amino]propyl}isoxazol-3-ol |
| 2958. | 5-{5-[[(1-butyl-4-chloro-2-phenyl-1H-imidazol-5-yl)methyl](2,3-dihydro-1,4- |
| | benzodioxin-6-ylmethyl)amino]pentyl}isoxazol-3-ol |
| 2959. | 4-{[[(1-butyl-4-chloro-2-phenyl-1H-imidazol-5-yl)methyl](2,3-dihydro-1,4- |
| | benzodioxin-6-ylmethyl)amino]methyl}-2-fluorobenzoic acid |
| 2960. | N-[(1-butyl-4-chloro-2-phenyl-1H-imidazol-5-yl)methyl]-N-(2,3-dihydro-1,4- |
| | benzodioxin-6-ylmethyl)-N-[(4-methyl-3,4-dihydro-2H-1,4-benzoxazin-6- |
| | yl)methyl]amine |
| 2961. | 6-{[[(1-butyl-4-chloro-2-phenyl-1H-imidazol-5-yl)methyl](2,3-dihydro-1,4- |
| | benzodioxin-6-ylmethyl)amino]methyl}nicotinic acid |
| 2962. | methyl 4-{[{[1-butyl-4-chloro-2-(2-methylphenyl)-1H-imidazol-5-yl]methyl}(2,3- |
| | dihydro-1,4-benzodioxin-6-ylmethyl)amino]methyl}benzoate |
| 2963. | 4-{[{[1-butyl-4-chloro-2-(2-methylphenyl)-1H-imidazol-5-yl]methyl}(2,3-dihydro-1,4- |
| | benzodioxin-6-ylmethyl)amino]methyl}benzoic acid |
| 2964. | methyl 4-{[[(4-chloro-2-phenyl-1-propyl-1H-imidazol-5-yl)methyl](2,3-dihydro-1,4- |
| | benzodioxin-6-ylmethyl)amino]methyl}benzoate |
| 2965. | methyl 4-{[{[1-butyl-2-phenyl-4-(trifluoromethyl)-1H-imidazol-5-yl]methyl}(2,3- |
| | dihydro-1,4-benzodioxin-6-ylmethyl)amino]methyl}benzoate |
| 2966. | methyl 4-{[[(1-butyl-4-cyano-2-phenyl-1H-imidazol-5-yl)methyl](2,3-dihydro-1,4- |
| | benzodioxin-6-ylmethyl)amino]methyl}benzoate |
| 2967. | methyl 4-{[[(1-butyl-2,4-diphenyl-1H-imidazol-5-yl)methyl](2,3-dihydro-1,4- |
| | benzodioxin-6-ylmethyl)amino]methyl}benzoate |
| 2968. | methyl 4-{[[(1-butyl-4-methyl-2-phenyl-1H-imidazol-5-yl)methyl](2,3-dihydro-1,4- |
| | benzodioxin-6-ylmethyl)amino]methyl}benzoate |
| 2969. | methyl 4-({(2,3-dihydro-1,4-benzodioxin-6-ylmethyl)[(4-methyl-2-phenyl-1-propyl- |
| | 1H-imidazol-5-yl)methyl]amino}methyl)benzoate |
| 2970. | 4-{[[(4-chloro-2-phenyl-1-propyl-1H-imidazol-5-yl)methyl](2,3-dihydro-1,4- |
| | benzodioxin-6-ylmethyl)amino]methyl}benzoic acid |
| 2971. | 4-{[{[1-butyl-2-phenyl-4-(trifluoromethyl)-1H-imidazol-5-yl]methyl}(2,3-dihydro-1,4- |
| | benzodioxin-6-ylmethyl)amino]methyl}benzoic acid |
| 2972. | 4-{[[(1-butyl-4-cyano-2-phenyl-1H-imidazol-5-yl)methyl](2,3-dihydro-1,4- |
| | benzodioxin-6-ylmethyl)amino]methyl}benzoic acid |
| 2973. | 4-{[[(1-butyl-2,4-diphenyl-1H-imidazol-5-yl)methyl](2,3-dihydro-1,4-benzodioxin-6- |
| | ylmethyl)amino]methyl}benzoic acid |
| 2974. | 4-{[[(1-butyl-4-methyl-2-phenyl-1H-imidazol-5-yl)methyl](2,3-dihydro-1,4- |
| | benzodioxin-6-ylmethyl)amino]methyl}benzoic acid |
| 2975. | 4-({(2,3-dihydro-1,4-benzodioxin-6-ylmethyl)[(4-methyl-2-phenyl-1-propyl-1H- |
| | imidazol-5-yl)methyl]amino}methyl)benzoic acid |
| 2976. | methyl 4-{[{[1-butyl-4-cyano-2-(2-methylphenyl)-1H-imidazol-5-yl]methyl}(2,3- |
| | dihydro-1,4-benzodioxin-6-ylmethyl)amino]methyl}benzoate |
| 2977. | methyl 4-{[{[1-butyl-4-fluoro-2-(2-methylphenyl)-1H-imidazol-5-yl]methyl}(3- |
| | ethoxybenzyl)amino]methyl}benzoate |
| 2978. | 4-{[{[1-butyl-4-cyano-2-(2-methylphenyl)-1H-imidazol-5-yl]methyl}(2,3-dihydro-1,4- |
| | benzodioxin-6-ylmethyl)amino]methyl}benzoic acid |
| 2979. | 4-{[{[1-butyl-4-fluoro-2-(2-methylphenyl)-1H-imidazol-5-yl]methyl}(3- |
| | ethoxybenzyl)amino]methyl}benzoic acid |
| 2980. | 4-{[{[1-butyl-4-fluoro-2-(2-methylphenyl)-1H-imidazol-5-yl]methyl}(2,3-dihydro-1,4- |
| | benzodioxin-6-ylmethyl)amino]methyl}benzoic acid |
| 2981. | methyl 4-{[{[1-butyl-4-fluoro-2-(2-methylphenyl)-1H-imidazol-5-yl]methyl}(2,3- |
| | dihydro-1,4-benzodioxin-6-ylmethyl)amino]methyl}benzoate |
| 2982. | N-{[1-butyl-4-chloro-2-(2-methylphenyl)-1H-imidazol-5-yl]methyl}-N-(1H-indol-5- |
| | ylmethyl)-3-methylbutan-1-amine |
| 2983. | N-{[1-butyl-4-chloro-2-(2-methylphenyl)-1H-imidazol-5-yl]methyl}-N-(1H-indol-5- |
| | ylmethyl)butan-1-amine |
| 2984. | methyl 4-{[[(1-butyl-2-phenyl-1H-imidazol-5-yl)methyl](2,3-dihydro-1,4-benzodioxin- |
| | 6-ylmethyl)amino]methyl}benzoate |
| 2985. | 4-{[[(1-butyl-2-phenyl-1H-imidazol-5-yl)methyl](2,3-dihydro-1,4-benzodioxin-6- |
| | ylmethyl)amino]methyl}benzoic acid |
| 2986. | methyl 4-{[[(4-bromo-1-butyl-2-phenyl-1H-imidazol-5-yl)methyl](2,3-dihydro-1,4- |
| | benzodioxin-6-ylmethyl)amino]methyl}benzoate |
| 2987. | 4-{[[(4-bromo-1-butyl-2-phenyl-1H-imidazol-5-yl)methyl](2,3-dihydro-1,4- |
| | benzodioxin-6-ylmethyl)amino]methyl}benzoic acid |
| 2988. | N-{[1-butyl-4-chloro-2-(2-methylphenyl)-1H-imidazol-5-yl]methyl}-N-(2,3-dihydro- |
| | 1,4-benzodioxin-6-ylmethyl)-3-methylbutan-1-amine |
| 2989. | methyl 4-{[{[1-butyl-4-chloro-2-(2-methylphenyl)-1H-imidazol-5- |
| | yl]methyl}(isopentyl)amino]methyl}benzoate |
| 2990. | 4-{[{[1-butyl-4-chloro-2-(2-methylphenyl)-1H-imidazol-5- |
| | yl]methyl}(isopentyl)amino]methyl}benzoic acid |
| 2991. | N-[(1-butyl-4-chloro-2-phenyl-1H-imidazol-5-yl)methyl]-N-(2,3-dihydro-1,4- |
| | benzodioxin-6-ylmethyl)-3-methylbutan-1-amine |
| 2992. | methyl 2-{[[(1-butyl-4-chloro-2-phenyl-1H-imidazol-5-yl)methyl](2,3-dihydro-1,4- |
| | benzodioxin-6-ylmethyl)amino]methyl}benzoate |
| 2993. | methyl 6-[[(1-butyl-4-chloro-2-phenyl-1H-imidazol-5-yl)methyl](2,3-dihydro-1,4- |
| | benzodioxin-6-ylmethyl)amino]hexanoate |
| 2994. | N-[(1-butyl-4-chloro-2-phenyl-1H-imidazol-5-yl)methyl]-N-(2,3-dihydro-1,4- |
| | benzodioxin-6-ylmethyl)prop-2-yn-1-amine |
| 2995. | N-[(1-butyl-4-chloro-2-phenyl-1H-imidazol-5-yl)methyl]-N-(2,3-dihydro-1,4- |
| | benzodioxin-6-ylmethyl)prop-2-en-1-amine |
| 2996. | ethyl 2-{[[(1-butyl-4-chloro-2-phenyl-1H-imidazol-5-yl)methyl](2,3-dihydro-1,4- |
| | benzodioxin-6-ylmethyl)amino]methyl}cyclopropanecarboxylate |
| 2997. | 2-{[[(1-butyl-4-chloro-2-phenyl-1H-imidazol-5-yl)methyl](2,3-dihydro-1,4- |
| | benzodioxin-6-ylmethyl)amino]methyl}benzoic acid |
| 2998. | 6-[[(1-butyl-4-chloro-2-phenyl-1H-imidazol-5-yl)methyl](2,3-dihydro-1,4-benzodioxin- |
| | 6-ylmethyl)amino]hexanoic acid |
| 2999. | ethyl 4-{3-[[(1-butyl-4-chloro-2-phenyl-1H-imidazol-5-yl)methyl](2,3-dihydro-1,4- |
| | benzodioxin-6-ylmethyl)amino]propyl}benzoate |
| 3000. | ethyl 4-(3-{butyl[(4-chloro-2-phenyl-1-propyl-1H-imidazol-5- |
| | yl)methyl]amino}propyl)benzoate |
| 3001. | ethyl 4-[3-(butyl{[1-butyl-4-chloro-2-(2-methylphenyl)-1H-imidazol-5- |
| | yl]methyl}amino)propyl]benzoate |
| 3002. | 4-{3-[[(1-butyl-4-chloro-2-phenyl-1H-imidazol-5-yl)methyl](2,3-dihydro-1,4- |
| | benzodioxin-6-ylmethyl)amino]propyl}benzoic acid |
| 3003. | 4-[3-(butyl{[1-butyl-4-chloro-2-(2-methylphenyl)-1H-imidazol-5- |
| | yl]methyl}amino)propyl]benzoic acid |
| 3004. | methyl (4-{[[(1-butyl-4-chloro-2-phenyl-1H-imidazol-5-yl)methyl](2,3-dihydro-1,4- |
| | benzodioxin-6-ylmethyl)amino]methyl}phenyl)acetate |
| 3005. | methyl 2-(4-{[[(1-butyl-4-chloro-2-phenyl-1H-imidazol-5-yl)methyl](2,3-dihydro-1,4- |
| | benzodioxin-6-ylmethyl)amino]methyl}phenyl)propanoate |
| 3006. | methyl 2-(4-{[[(1-butyl-4-chloro-2-phenyl-1H-imidazol-5-yl)methyl](2,3-dihydro-1,4- |
| | benzodioxin-6-ylmethyl)amino]methyl}phenyl)-2-methylpropanoate |
| 3007. | methyl (3-{[[(1-butyl-4-chloro-2-phenyl-1H-imidazol-5-yl)methyl](2,3-dihydro-1,4- |
| | benzodioxin-6-ylmethyl)amino]methyl}phenyl)acetate |
| 3008. | (4-{[[(1-butyl-4-chloro-2-phenyl-1H-imidazol-5-yl)methyl](2,3-dihydro-1,4- |
| | benzodioxin-6-ylmethyl)amino]methyl}phenyl)acetic acid |
| 3009. | 2-(4-{[[(1-butyl-4-chloro-2-phenyl-1H-imidazol-5-yl)methyl](2,3-dihydro-1,4- |
| | benzodioxin-6-ylmethyl)amino]methyl}phenyl)propanoic acid |
| 3010. | 2-(4-{[[(1-butyl-4-chloro-2-phenyl-1H-imidazol-5-yl)methyl](2,3-dihydro-1,4- |
| | benzodioxin-6-ylmethyl)amino]methyl}phenyl)-2-methylpropanoic acid |
| 3011. | (3-{[[(1-butyl-4-chloro-2-phenyl-1H-imidazol-5-yl)methyl](2,3-dihydro-1,4- |
| | benzodioxin-6-ylmethyl)amino]methyl}phenyl)acetic acid |
| 3012. | N-{[1-butyl-4-chloro-2-(2-methylphenyl)-1H-imidazol-5-yl]methyl}-N-(2,3-dihydro- |
| | 1,4-benzodioxin-6-ylmethyl)butan-1-amine |
| 3013. | 4-[(butyl{[1-butyl-4-chloro-2-(2-methylphenyl)-1H-imidazol-5- |
| | yl]methyl}amino)methyl]benzenesulfonamide |
| 3014. | 4-{[{[1-butyl-4-chloro-2-(2-methylphenyl)-1H-imidazol-5- |
| | yl]methyl}(isopentyl)amino]methyl}benzenesulfonamide |
| 3015. | 4-{[[(1-butyl-4-chloro-2-phenyl-1H-imidazol-5-yl)methyl](2,3-dihydro-1,4- |
| | benzodioxin-6-ylmethyl)amino]methyl}benzenesulfonamide |
| 3016. | N-{[1-butyl-4-chloro-2-(2-methylphenyl)-1H-imidazol-5-yl]methyl}-N-[(3-chloro-1H- |
| | indol-5-yl)methyl]-3-methylbutan-1-amine |
| 3017. | 5-{[{[1-butyl-4-chloro-2-(2-methylphenyl)-1H-imidazol-5- |
| | yl]methyl}(isopentyl)amino]methyl}-1H-indole-3-carbonitrile |
| 3018. | 4-{[{[1-butyl-4-chloro-2-(2-methylphenyl)-1H-imidazol-5-yl]methyl}(2,3-dihydro-1,4- |
| | benzodioxin-6-ylmethyl)amino]methyl}benzenesulfonamide |
| 3019. | N-{[1-butyl-4-chloro-2-(2-methylphenyl)-1H-imidazol-5-yl]methyl}-N-(quinoxalin-6- |
| | ylmethyl)butan-1-amine |
| 3020. | 5-(5-{butyl[(1-butyl-4-chloro-2-phenyl-1H-imidazol-5- |
| | yl)methyl]amino}pentyl)isoxazol-3-ol |
| 3021. | methyl [[(1-butyl-4-chloro-2-phenyl-1H-imidazol-5-yl)methyl](2,3-dihydro-1,4- |
| | benzodioxin-6-ylmethyl)amino](phenyl)acetate |
| 3022. | N-[(1-butyl-2-phenyl-4-vinyl-1H-imidazol-5-yl)methyl]-N-(2,3-dihydro-1,4- |
| | benzodioxin-6-ylmethyl)-N-(3-ethoxybenzyl)amine |
| 3023. | methyl 4-{[[(1-butyl-2-phenyl-4-vinyl-1H-imidazol-5-yl)methyl](2,3-dihydro-1,4- |
| | benzodioxin-6-ylmethyl)amino]methyl}benzoate |
| 3024. | N-[(1-butyl-4-phenyl-2-vinyl-1H-imidazol-5-yl)methyl]-N-(2,3-dihydro-1,4- |
| | benzodioxin-6-ylmethyl)-N-(3-ethoxybenzyl)amine |
| 3025. | N-[(1-butyl-4-ethyl-2-phenyl-1H-imidazol-5-yl)methyl]-N-(2,3-dihydro-1,4- |
| | benzodioxin-6-ylmethyl)-N-(3-ethoxybenzyl)amine |
| 3026. | methyl 4-{[[(1-butyl-4-ethyl-2-phenyl-1H-imidazol-5-yl)methyl](2,3-dihydro-1,4- |
| | benzodioxin-6-ylmethyl)amino]methyl}benzoate |
| 3027. | (1S)-N-(1,3-benzodioxol-5-ylmethyl)-1-(1-butyl-2,4-diphenyl-1H-imidazol-5-yl)-N- |
| | methylpentan-1-amine |
| 3028. | N-[(1-butyl-4-chloro-2-phenyl-1H-imidazol-5-yl)methyl]-N-(2,3-dihydro-1,4- |
| | benzodioxin-6-ylmethyl)-N-(quinoxalin-6-ylmethyl)amine |
| 3029. | 4-{[[(1-butyl-2-phenyl-4-vinyl-1H-imidazol-5-yl)methyl](2,3-dihydro-1,4-benzodioxin- |
| | 6-ylmethyl)amino]methyl}benzoic acid |
| 3030. | N-{1-[1-butyl-4-chloro-2-(2-methylphenyl)-1H-imidazol-5-yl]ethyl}-N-(2,3-dihydro- |
| | 1,4-benzodioxin-6-ylmethyl)butan-1-amine |
| 3031. | N-{1-[1-butyl-4-chloro-2-(2-methylphenyl)-1H-imidazol-5-yl]ethyl}-N-(2,3-dihydro- |
| | 1,4-benzodioxin-6-ylmethyl)-3-methylbutan-1-amine |
| 3032. | N-{1-[1-butyl-4-chloro-2-(2-methylphenyl)-1H-imidazol-5-yl]ethyl}-N-(1H-indol-5- |
| | ylmethyl)butan-1-amine |
| 3033. | N-{[1-butyl-4-chloro-2-(2-methylphenyl)-1H-imidazol-5-yl]methyl}-3-methyl-N-[(2- |
| | methyl-1H-indol-5-yl)methyl]butan-1-amine |
| 3034. | 1-(1-butyl-2-phenyl-1H-imidazol-5-yl)-N-(cyclohexylmethyl)-N-methylpentan-1-amine |
| 3035. | 4-{[[(1-butyl-4-ethyl-2-phenyl-1H-imidazol-5-yl)methyl](2,3-dihydro-1,4-benzodioxin- |
| | 6-ylmethyl)amino]methyl}benzoic acid |
| 3036. | N-{[4-chloro-1-(ethoxymethyl)-2-phenyl-1H-imidazol-5-yl]methyl}-N-(2,3-dihydro- |
| | 1,4-benzodioxin-6-ylmethyl)-N-(3-ethoxybenzyl)amine |
| 3037. | 1-(4-chloro-2-phenyl-1H-imidazol-5-yl)-N-(2,3-dihydro-1,4-benzodioxin-6-ylmethyl)- |
| | N-(3-ethoxybenzyl)methanamine |
| 3038. | N-{[1-butyl-4-chloro-2-(2-methylphenyl)-1H-imidazol-5-yl]methyl}-N-methyl-1,2,3,4- |
| | tetrahydronaphthalen-1-amine |
| 3039. | 1-(1-butyl-2,4-diphenyl-1H-imidazol-5-yl)-N-(cyclohexylmethyl)-N-(1,2,3,4- |
| | tetrahydroquinolin-6-ylmethyl)methanamine |
| 3040. | 1-[1-butyl-2-phenyl-4-(trifluoromethyl)-1H-imidazol-5-yl]-N-(cyclohexylmethyl)-N- |
| | (1,2,3,4-tetrahydroquinolin-6-ylmethyl)methanamine |
| 3041. | N-{[4-bromo-1-butyl-2-(2,6-diethylphenyl)-1H-imidazol-5-yl]methyl}-N-methyl- |
| | 1,2,3,4-tetrahydronaphthalen-1-amine |
| 3042. | methyl 4-{[{[1-butyl-4-(4-methylphenyl)-2-phenyl-1H-imidazol-5-yl]methyl}(2,3- |
| | dihydro-1,4-benzodioxin-6-ylmethyl)amino]methyl}benzoate |
| 3043. | methyl 4-{[{[1-butyl-4-(3-fluorophenyl)-2-phenyl-1H-imidazol-5-yl]methyl}(2,3- |
| | dihydro-1,4-benzodioxin-6-ylmethyl)amino]methyl}benzoate |
| 3044. | 4-{[{[1-butyl-4-(4-methylphenyl)-2-phenyl-1H-imidazol-5-yl]methyl}(2,3-dihydro-1,4- |
| | benzodioxin-6-ylmethyl)amino]methyl}benzoic acid |
| 3045. | 4-{[{[1-butyl-4-(3-fluorophenyl)-2-phenyl-1H-imidazol-5-yl]methyl}(2,3-dihydro-1,4- |
| | benzodioxin-6-ylmethyl)amino]methyl}benzoic acid |
| 3046. | N-{[1-butyl-4-chloro-2-(2-methylphenyl)-1H-imidazol-5-yl]methyl}-N-(3- |
| | methoxybenzyl)butan-1-amine |
| 3047. | N-{[1-butyl-4-chloro-2-(2-methylphenyl)-1H-imidazol-5-yl]methyl}-N-(3- |
| | ethoxybenzyl)butan-1-amine |
| 3048. | N-{[1-butyl-4-chloro-2-(2-methylphenyl)-1H-imidazol-5-yl]methyl}-N-(1H-indol-5- |
| | ylmethyl)-3,3-dimethylbutan-1-amine |
| 3049. | 6-[(butyl{[1-butyl-4-chloro-2-(2-methylphenyl)-1H-imidazol-5- |
| | yl]methyl}amino)methyl]-3,4-dihydroquinolin-2(1H)-one |
| 3050. | N-{[1-butyl-4-chloro-2-(2-methylphenyl)-1H-imidazol-5-yl]methyl}-N-(1,2,3,4- |
| | tetrahydroquinolin-6-ylmethyl)butan-1-amine |
| 3051. | N-{[1-butyl-4-chloro-2-(2,6-diethylphenyl)-1H-imidazol-5-yl]methyl}-N-methyl- |
| | 1,2,3,4-tetrahydronaphthalen-1-amine |
| 3052. | (1S)-N-{[1-butyl-4-chloro-2-(2,6-diethylphenyl)-1H-imidazol-5-yl]methyl}-N-methyl- |
| | 1,2,3,4-tetrahydronaphthalen-1-amine |
| 3053. | 6-{[{[1-butyl-4-chloro-2-(2-methylphenyl)-1H-imidazol-5- |
| | yl]methyl}(isopentyl)amino]methyl}-3,4-dihydroquinolin-2(1H)-one |
| 3054. | N-{[1-butyl-4-chloro-2-(2-methylphenyl)-1H-imidazol-5-yl]methyl}-N-(2,3-dihydro-1- |
| | benzofuran-5-ylmethyl)-3-methylbutan-1-amine |
| 3055. | N-{[1-butyl-4-chloro-2-(2-methylphenyl)-1H-imidazol-5-yl]methyl}-N-(4- |
| | methoxybenzyl)-3-methylbutan-1-amine |
| 3056. | N-{[1-butyl-4-chloro-2-(2-methylphenyl)-1H-imidazol-5-yl]methyl}-N-(3- |
| | methoxybenzyl)-3-methylbutan-1-amine |
| 3057. | N-{[1-butyl-4-chloro-2-(2-methylphenyl)-1H-imidazol-5-yl]methyl}-N-(4- |
| | ethoxybenzyl)-3-methylbutan-1-amine |
| 3058. | N-{[1-butyl-4-chloro-2-(2-methylphenyl)-1H-imidazol-5-yl]methyl}-N-(3- |
| | ethoxybenzyl)-3-methylbutan-1-amine |
| 3059. | ethyl N-{[1-butyl-4-chloro-2-(2-methylphenyl)-1H-imidazol-5-yl]methyl}-N-(2,3- |
| | dihydro-1,4-benzodioxin-6-ylmethyl)-beta-alaninate |
| 3060. | 4-[{[1-butyl-4-chloro-2-(2-methylphenyl)-1H-imidazol-5-yl]methyl}(2,3-dihydro-1,4- |
| | benzodioxin-6-ylmethyl)amino]-2-methylbutan-2-ol |
| 3061. | ethyl N-{[1-butyl-4-chloro-2-(2-methylphenyl)-1H-imidazol-5-yl]methyl}-N-(1H- |
| | indol-5-ylmethyl)-beta-alaninate |
| 3062. | 4-[{[1-butyl-4-chloro-2-(2-methylphenyl)-1H-imidazol-5-yl]methyl}(1H-indol-5- |
| | ylmethyl)amino]-2-methylbutan-2-ol |
| 3063. | N-{[1-butyl-4-chloro-2-(2-methylphenyl)-1H-imidazol-5-yl]methyl}-N-[(3-chloro-1H- |
| | indol-5-yl)methyl]-3,3-dimethylbutan-1-amine |
| 3064. | N-{[1-butyl-2-(2,6-diethylphenyl)-4-(4-methylphenyl)-1H-imidazol-5-yl]methyl}-N- |
| | methyl-1,2,3,4-tetrahydronaphthalen-1-amine |
| 3065. | N-{[1-butyl-2-(2,6-diethylphenyl)-1H-imidazol-5-yl]methyl}-N-methyl-1,2,3,4- |
| | tetrahydronaphthalen-1-amine |
| 3066. | N-{[1-butyl-2-(2,6-diethylphenyl)-4-methyl-1H-imidazol-5-yl]methyl}-N-methyl- |
| | 1,2,3,4-tetrahydronaphthalen-1-amine |
| 3067. | methyl 4-{[{[1-butyl-4-(4-fluorophenyl)-2-phenyl-1H-imidazol-5-yl]methyl }(2,3- |
| | dihydro-1,4-benzodioxin-6-ylmethyl)amino]methyl}benzoate |
| 3068. | methyl 4-{[{[1-butyl-4-(3-methylphenyl)-2-phenyl-1H-imidazol-5-yl]methyl}(2,3- |
| | dihydro-1,4-benzodioxin-6-ylmethyl)amino]methyl}benzoate |
| 3069. | methyl 4-{[{[1-butyl-4-(3-methoxyphenyl)-2-phenyl-1H-imidazol-5-yl]methyl}(2,3- |
| | dihydro-1,4-benzodioxin-6-ylmethyl)amino]methyl}benzoate |
| 3070. | methyl 4-{[{[1-butyl-4-(4-methoxyphenyl)-2-phenyl-1H-imidazol-5-yl]methyl}(2,3- |
| | dihydro-1,4-benzodioxin-6-ylmethyl)amino]methyl}benzoate |
| 3071. | methyl 4-{[{[1-butyl-4-(4-chlorophenyl)-2-phenyl-1H-imidazol-5-yl]methyl}(2,3- |
| | dihydro-1,4-benzodioxin-6-ylmethyl)amino]methyl}benzoate |
| 3072. | 4-{{[1-butyl-4-chloro-2-(2-methylphenyl)-1H-imidazol-5-yl]methyl}[(3-chloro-1H- |
| | indol-5-yl)methyl]amino}1-2-methylbutan-2-ol |
| 3073. | N-{[1-butyl-4-chloro-2-(2-methylphenyl)-1H-imidazol-5-yl]methyl}-N-(1H-indol-5- |
| | ylmethyl)-2,2-dimethylpropan-1-amine |
| 3074. | N-{[1-butyl-4-chloro-2-(2-methylphenyl)-1H-imidazol-5-yl]methyl}-N-[(3-chloro-1H- |
| | indol-5-yl)methyl]-2,2-dimethylpropan-1-amine |
| 3075. | N-{[1-butyl-4-chloro-2-(2-methylphenyl)-1H-imidazol-5-yl]methyl}-N-(2,3-dihydro- |
| | 1,4-benzodioxin-6-ylmethyl)-2,2-dimethylpropan-1-amine |
| 3076. | N-{[1-butyl-4-chloro-2-(2-methylphenyl)-1H-imidazol-5-yl]methyl}-N-(2-fluoro-4- |
| | methoxybenzyl)-3-methylbutan-1-amine |
| 3077. | N-{[1-butyl-4-chloro-2-(2-methylphenyl)-1H-imidazol-5-yl]methyl}-N-(2-chloro-4- |
| | methoxybenzyl)-3-methylbutan-1-amine |
| 3078. | N-{[4-bromo-2-(2,6-diethylphenyl)-1-(1,3-dioxolan-2-ylmethyl)-1H-imidazol-5- |
| | yl]methyl}-N-methyl-1,2,3,4-tetrahydronaphthalen-1-amine |
| 3079. | N-{[4-bromo-2-(2,6-diethylphenyl)-1-(ethoxymethyl)-1H-imidazol-5-yl]methyl}-N- |
| | methyl-1,2,3,4-tetrahydronaphthalen-1-amine |
| 3080. | 4-{[{[1-butyl-4-(4-fluorophenyl)-2-phenyl-1H-imidazol-5-yl]methyl}(2,3-dihydro-1,4- |
| | benzodioxin-6-ylmethyl)amino]methyl}benzoic acid |
| 3081. | 4-{[{[1-butyl-4-(3-methylphenyl)-2-phenyl-1H-imidazol-5-yl]methyl}(2,3-dihydro-1,4- |
| | benzodioxin-6-ylmethyl)amino]methyl}benzoic acid |
| 3082. | 4-{[{[1-butyl-4-(3-methoxyphenyl)-2-phenyl-1H-imidazol-5-yl]methyl}(2,3-dihydro- |
| | 1,4-benzodioxin-6-ylmethyl)amino]methyl}benzoic acid |
| 3083. | 4-{[{[1-butyl-4-(4-chlorophenyl)-2-phenyl-1H-imidazol-5-yl]methyl}(2,3-dihydro-1,4- |
| | benzodioxin-6-ylmethyl)amino]methyl}benzoic acid |
| 3084. | 4-{[{[1-butyl-4-(4-methoxyphenyl)-2-phenyl-1H-imidazol-5-yl]methyl}(2,3-dihydro- |
| | 1,4-benzodioxin-6-ylmethyl)amino]methyl}benzoic acid |
| 3085. | N-{[4-bromo-2-(2,6-diethylphenyl)-1-(2-morpholin-4-ylethyl)-1H-imidazol-5- |
| | yl]methyl}-N-methyl-1,2,3,4-tetrahydronaphthalen-1-amine |
| 3086. | N-{[4-bromo-2-(2,6-diethylphenyl)-1H-imidazol-5-yl]methyl}-N-methyl-1,2,3,4- |
| | tetrahydronaphthalen-1-amine |
| 3087. | 1-[2-(2,6-diethylphenyl)-4-methoxy-6-methylpyrimidin-5-yl]-N-methyl-N-[(8- |
| | methylimidazo[1,2-a]pyridin-2-yl)methyl]methanamine |
| 3088. | N-{[4-chloro-1-(2-morpholin-4-ylethyl)-2-phenyl-1H-imidazol-5-yl]methyl}-N-(2,3- |
| | dihydro-1,4-benzodioxin-6-ylmethyl)-N-(3-ethoxybenzyl)amine |
| 3089. | 1-[4-chloro-2-phenyl-1-(2-piperidin-1-ylethyl)-1H-imidazol-5-yl]-N-(2,3-dihydro-1,4- |
| | benzodioxin-6-ylmethyl)-N-(3-ethoxybenzyl)methanamine |
| 3090. | methyl 4-[(butyl{[1-butyl-2-(2-methylphenyl)-4-(4-methylphenyl)-1H-imidazol-5- |
| | yl]methyl}amino)methyl]benzoate |
| 3091. | methyl 4-{[{[1-butyl-2-(2-methylphenyl)-4-(4-methylphenyl)-1H-imidazol-5- |
| | yl]methyl}(isobutyl)amino]methyl}benzoate |
| 3092. | methyl 4-{[{[1-butyl-2-(2-methylphenyl)-4-(4-methylphenyl)-1H-imidazol-5- |
| | yl]methyl}(2-ethylbutyl)amino]methyl}benzoate |
| 3093. | methyl 4-{[{[1-butyl-2-(2-methylphenyl)-4-(4-methylphenyl)-1H-imidazol-5- |
| | yl]methyl}(isopentyl)amino]methyl}benzoate |
| 3094. | methyl 4-{[{[1-butyl-2-(2-methylphenyl)-4-(4-methylphenyl)-1H-imidazol-5- |
| | yl]methyl}(cyclohexylmethyl)amino]methyl}benzoate |
| 3095. | methyl 4-[(benzyl{[1-butyl-2-(2-methylphenyl)-4-(4-methylphenyl)-1H-imidazol-5- |
| | yl]methyl}amino)methyl]benzoate |
| 3096. | methyl 4-{[{[1-butyl-2-(2-methylphenyl)-4-(4-methylphenyl)-1H-imidazol-5- |
| | yl]methyl}(2,3-dihydro-1,4-benzodioxin-6-ylmethyl)amino]methyl}benzoate |
| 3097. | methyl 4-{[{[1-butyl-2-(2-methylphenyl)-4-phenyl-1H-imidazol-5- |
| | yl]methyl}(isopentyl)amino]methyl}benzoate |
| 3098. | methyl 4-{[{[1-butyl-2-(2-methylphenyl)-4-phenyl-1H-imidazol-5-yl]methyl}(2,3- |
| | dihydro-1,4-benzodioxin-6-ylmethyl)amino]methyl}benzoate |
| 3099. | methyl 4-[(benzyl{[1-butyl-2-(2-methylphenyl)-4-phenyl-1H-imidazol-5- |
| | yl]methyl}amino)methyl]benzoate |
| 3100. | 4-[(butyl{[1-butyl-2-(2-methylphenyl)-4-(4-methylphenyl)-1H-imidazol-5- |
| | yl]methyl}amino)methyl]benzoic acid |
| 3101. | 4-{[{[1-butyl-2-(2-methylphenyl)-4-(4-methylphenyl)-1H-imidazol-5- |
| | yl]methyl}(isobutyl)amino]methyl}benzoic acid |
| 3102. | 4-{[{[1-butyl-2-(2-methylphenyl)-4-(4-methylphenyl)-1H-imidazol-5-yl]methyl}(2- |
| | ethylbutyl)amino]methyl}benzoic acid |
| 3103. | 4-{[{[1-butyl-2-(2-methylphenyl)-4-(4-methylphenyl)-1H-imidazol-5- |
| | yl]methyl}(isopentyl)amino]methyl}benzoic acid |
| 3104. | 4-{[{[1-butyl-2-(2-methylphenyl)-4-(4-methylphenyl)-1H-imidazol-5- |
| | yl]methyl}(cyclohexylmethyl)amino]methyl}benzoic acid |
| 3105. | 4-[(benzyl{[1-butyl-2-(2-methylphenyl)-4-(4-methylphenyl)-1H-imidazol-5- |
| | yl]methyl}amino)methyl]benzoic acid |
| 3106. | N-{[4-chloro-2-(2,6-dimethylphenyl)-1-methyl-1H-imidazol-5-yl]methyl}-N-methyl- |
| | 1,2,3,4-tetrahydronaphthalen-1-amine |
| 3107. | 4-{[{[1-butyl-2-(2-methylphenyl)-4-(4-methylphenyl)-1H-imidazol-5-yl]methyl}(2,3- |
| | dihydro-1,4-benzodioxin-6-ylmethyl)amino]methyl}benzoic acid |
| 3108. | 4-{[{[1-butyl-2-(2-methylphenyl)-4-phenyl-1H-imidazol-5- |
| | yl]methyl}(isopentyl)amino]methyl}benzoic acid |
| 3109. | 4-{[{[1-butyl-2-(2-methylphenyl)-4-phenyl-1H-imidazol-5-yl]methyl}(2,3-dihydro-1,4- |
| | benzodioxin-6-ylmethyl)amino]methyl}benzoic acid |
| 3110. | 4-[(benzyl{[1-butyl-2-(2-methylphenyl)-4-phenyl-1H-imidazol-5- |
| | yl]methyl}amino)methyl]benzoic acid |
| 3111. | 4-[(butyl{[1-butyl-2-(2-methylphenyl)-4-phenyl-1H-imidazol-5- |
| | yl]methyl}amino)methyl]benzoic acid |
| 3112. | methyl 4-{[{[1-butyl-2-(2-methylphenyl)-4-(4-methylphenyl)-1H-imidazol-5- |
| | yl]methyl}(propyl)amino]methyl}benzoate |
| 3113. | methyl 4-{[{[1-butyl-2-(2-methylphenyl)-4-(4-methylphenyl)-1H-imidazol-5- |
| | yl]methyl}(neopentyl)amino]methyl}benzoate |
| 3114. | methyl 4-{[{[1-butyl-2-(2-methylphenyl)-4-(4-methylphenyl)-1H-imidazol-5- |
| | yl]methyl}(3,3-dimethylbutyl)amino]methyl}benzoate |
| 3115. | methyl 4-{[{[1-butyl-2-(2-methylphenyl)-4-(4-methylphenyl)-1H-imidazol-5- |
| | yl]methyl}(pentyl)amino]methyl}benzoate |
| 3116. | methyl 4-{[{[1-butyl-2-(2-methylphenyl)-4-(4-methylphenyl)-1H-imidazol-5- |
| | yl]methyl}(hexyl)amino]methyl}benzoate |
| 3117. | methyl 4-{[{[1-butyl-2-(2-methylphenyl)-4-(4-methylphenyl)-1H-imidazol-5- |
| | yl]methyl}(3-methylbut-2-enyl)amino]methyl}benzoate |
| 3118. | methyl 4-[((bicyclo[2.2.1]hept-5-en-2-ylmethyl){[1-butyl-2-(2-methylphenyl)-4-(4- |
| | methylphenyl)-1H-imidazol-5-yl]methyl}amino)methyl]benzoate |
| 3119. | methyl 4-{[{[1-butyl-2-(2-methylphenyl)-4-(4-methylphenyl)-1H-imidazol-5- |
| | yl]methyl}(tetrahydrofuran-3-ylmethyl)amino]methyl}benzoate |
| 3120. | methyl 4-{[{[1-butyl-2-(2-methylphenyl)-4-(4-methylphenyl)-1H-imidazol-5- |
| | yl]methyl}(thien-3-ylmethyl)amino]methyl}benzoate |
| 3121. | 4-{[{[1-butyl-2-(2-methylphenyl)-4-(4-methylphenyl)-1H-imidazol-5- |
| | yl]methyl}(propyl)amino]methyl}benzoic acid |
| 3122. | 4-{[{[1-butyl-2-(2-methylphenyl)-4-(4-methylphenyl)-1H-imidazol-5- |
| | yl]methyl}(neopentyl)amino]methyl}benzoic acid |
| 3123. | 4-{[{[1-butyl-2-(2-methylphenyl)-4-(4-methylphenyl)-1H-imidazol-5-yl]methyl}(3,3- |
| | dimethylbutyl)amino]methyl}benzoic acid |
| 3124. | 4-{[{[1-butyl-2-(2-methylphenyl)-4-(4-methylphenyl)-1H-imidazol-5- |
| | yl]methyl}(pentyl)amino]methyl}benzoic acid |
| 3125. | 4-{[{[1-butyl-2-(2-methylphenyl)-4-(4-methylphenyl)-1H-imidazol-5- |
| | yl]methyl}(hexyl)amino]methyl}benzoic acid |
| 3126. | 4-{[{[1-butyl-2-(2-methylphenyl)-4-(4-methylphenyl)-1H-imidazol-5-yl]methyl}(3- |
| | methylbut-2-enyl)amino]methyl}benzoic acid |
| 3127. | 4-[((bicyclo[2.2.1]hept-5-en-2-ylmethyl){[1-butyl-2-(2-methylphenyl)-4-(4- |
| | methylphenyl)-1H-imidazol-5-yl]methyl}amino)methyl]benzoic acid |
| 3128. | 4-{[{[1-butyl-2-(2-methylphenyl)-4-(4-methylphenyl)-1H-imidazol-5-yl]methyl}(thien- |
| | 3-ylmethyl)amino]methyl}benzoic acid |
| 3129. | methyl 4-{[{[1-butyl-2-(2-methylphenyl)-4-(4-methylphenyl)-1H-imidazol-5- |
| | yl]methyl}(1H-indol-5-ylmethyl)amino]methyl}benzoate |
| 3130. | 4-{[{[1-butyl-2-(2-methylphenyl)-4-(4-methylphenyl)-1H-imidazol-5-yl]methyl}(1H- |
| | indol-5-ylmethyl)amino]methyl}benzoic acid |
| 3131. | N-{[1-butyl-4-chloro-2-(2-methylphenyl)-1H-imidazol-5-yl]methyl}-N-(1H-indazol-5- |
| | ylmethyl)-3-methylbutan-1-amine |
| 3132. | 4-{[{[1-butyl-4-(4-methoxyphenyl)-2-phenyl-1H-imidazol-5- |
| | yl]methyl}(ethyl)amino]methyl}-2-hydroxybenzamide |
| 3133. | (1S)-N-{[1-butyl-4-chloro-2-(2,6-dimethylphenyl)-1H-imidazol-5-yl]methyl}-N- |
| | methyl-1,2,3,4-tetrahydronaphthalen-1-amine |
| 3134. | N-{[1-butyl-4-chloro-2-(2-methylphenyl)-1H-imidazol-5-yl]methyl}-N-(1H-indazol-5- |
| | ylmethyl)-2,2-dimethylpropan-1-amine |
| 3135. | 1-[1-butyl-4-chloro-2-(2-methylphenyl)-1H-imidazol-5-yl]-N-(1H-indazol-5-ylmethyl)- |
| | N-(3-methoxybenzyl)methanamine |
| 3136. | 1-[1-butyl-4-chloro-2-(2-methylphenyl)-1H-imidazol-5-yl]-N-(1H-indazol-5-ylmethyl)- |
| | N-(4-methoxybenzyl)methanamine |
| 3137. | 4-{[{[1-butyl-4-chloro-2-(2-methylphenyl)-1H-imidazol-5-yl]methyl}(4- |
| | methoxybenzyl)amino]methyl}-2-hydroxybenzamide |
| 3138. | 4-{[{[1-butyl-4-chloro-2-(2-methylphenyl)-1H-imidazol-5-yl]methyl}(3- |
| | methoxybenzyl)amino]methyl}-2-hydroxybenzamide |
| 3139. | 4-{[{[1-butyl-4-(4-methoxyphenyl)-2-phenyl-1H-imidazol-5-yl]methyl}(2- |
| | methoxyethyl)amino]methyl}-2-hydroxybenzamide |
| 3140. | 4-{[{[1-butyl-4-(4-methoxyphenyl)-2-pyridin-3-yl-1H-imidazol-5- |
| | yl]methyl}(cyclohexylmethyl)amino]methyl}-2-hydroxybenzamide |
| 3141. | 7-{[{[1-butyl-4-(4-methoxyphenyl)-2-phenyl-1H-imidazol-5- |
| | yl]methyl}(neopentyl)amino]methyl}-2H-1,3-benzoxazine-2,4(3H)-dione |
| 3142. | 1-(1-butyl-2,4-diphenyl-1H-imidazol-5-yl)-N-(cyclohexylmethyl)-N-(1H-indazol-5- |
| | ylmethyl)methanamine |
| 3143. | N-[(1-butyl-2,4-diphenyl-1H-imidazol-5-yl)methyl]-N-(1H-indazol-5-ylmethyl)-2,2- |
| | dimethylpropan-1-amine |
| 3144. | 1-[1-butyl-4-(4-methoxyphenyl)-2-phenyl-1H-imidazol-5-yl]-N-(cyclohexylmethyl)-N- |
| | (1H-indazol-5-ylmethyl)methanamine |
| 3145. | methyl 4-[((cyclohexylmethyl){[1-[2-(dimethylamino)ethyl]-4-(4-methoxyphenyl)-2- |
| | phenyl-1H-imidazol-5-yl]methyl}amino)methyl]-2-methoxybenzoate |
| 3146. | N-{[4-chloro-2-(2,6-diethylphenyl)-1-(2-methoxyethyl)-1H-imidazol-5-yl]methyl}-N- |
| | propylpropan-1-amine |
| 3147. | 4-[((cyclohexylmethyl){[1-[2-(dimethylamino)ethyl]-4-(4-methoxyphenyl)-2-phenyl- |
| | 1H-imidazol-5-yl]methyl}amino)methyl]-2-hydroxybenzamide |
| 3148. | 1-butyl-5-{[(1H-indazol-5-ylmethyl)(neopentyl)amino]methyl}-2-(2-methylphenyl)- |
| | 1H-imidazole-4-carbonitrile |
| 3149. | 1-butyl-5-{[(cyclohexylmethyl)(1H-indazol-5-ylmethyl)amino]methyl}-2-(2- |
| | methylphenyl)-1H-imidazole-4-carbonitrile |
| 3150. | N-{[4-chloro-2-(2,6-diethylphenyl)-1-(2-methoxyethyl)-1H-imidazol-5-yl]methyl}-N- |
| | (4-methoxybenzyl)-2,2-dimethylpropan-1-amine |
| 3151. | 4-{[{[1-butyl-4-cyano-2-(2-methylphenyl)-1H-imidazol-5- |
| | yl]methyl}(cyclohexylmethyl)amino]methyl}-2-hydroxybenzamide |
| 3152. | N-{[4-chloro-2-(2,6-diethylphenyl)-1-(2-methoxyethyl)-1H-imidazol-5-yl]methyl}-N- |
| | (3-ethoxybenzyl)-2,2-dimethylpropan-1-amine |
| 3153. | N-{[4-chloro-2-(2,6-diethylphenyl)-1-(2-methoxyethyl)-1H-imidazol-5-yl]methyl}-N- |
| | (cyclohexylmethyl)-2,2-dimethylpropan-1-amine |
| 3154. | N-{[4-chloro-2-(2,6-diethylphenyl)-1-(2-methoxyethyl)-1H-imidazol-5-yl]methyl}-N- |
| | (cyclohexylmethyl)-2-methylpropan-2-amine |
| 3155. | 1-(5-butyl-3-morpholin-4-yl-6-phenylpyridazin-4-yl)-N-(2,3-dihydro-1,4-benzodioxin- |
| | 6-ylmethyl)-N-(3-ethoxybenzyl)methanamine |
| 3156. | N-1-(5-butyl-4-{[(2,3-dihydro-1,4-benzodioxin-6-ylmethyl)(3- |
| | ethoxybenzyl)amino]methyl}-6-phenylpyridazin-3-yl)-N-2-,N-2-dimethylethane-1,2- |
| | diamine |
| 3157. | N-[(5-butyl-6-phenylpyrimidin-4-yl)methyl]-N-(2,3-dihydro-1,4-benzodioxin-6- |
| | ylmethyl)-N-(3-ethoxybenzyl)amine |
| 3158. | 1-(5-butyl-6-phenyl-3-pyrrolidin-1-ylpyridazin-4-yl)-N-(2,3-dihydro-1,4-benzodioxin- |
| | 6-ylmethyl)-N-methylmethanamine |
| 3159. | 1-(5-butyl-6-phenyl-3-piperidin-1-ylpyridazin-4-yl)-N-(2,3-dihydro-1,4-benzodioxin-6- |
| | ylmethyl)-N-methylmethanamine |
| 3160. | 5-butyl-4-{[(2,3-dihydro-1,4-benzodioxin-6-ylmethyl)(methyl)amino]methyl}-6- |
| | phenyl-N,N-dipropylpyridazin-3-amine |
| 3161. | 1-[3-chloro-5-methyl-6-(2-methylphenyl)pyridazin-4-yl]-N-(2,3-dihydro-1,4- |
| | benzodioxin-6-ylmethyl)-N-(3-ethoxybenzyl)methanamine |
| 3162. | 1-(2,3-dihydro-1,4-benzodioxin-6-yl)-N-(3-ethoxybenzyl)-N-{[5-methyl-6-(2- |
| | methylphenyl)-3-pyrrolidin-1-ylpyridazin-4-yl]methyl}methanamine |
| 3163. | 1-(2,3-dihydro-1,4-benzodioxin-6-yl)-N-(3-ethoxybenzyl)-N-{[5-methyl-6-(2- |
| | methylphenyl)-3-piperidin-1-ylpyridazin-4-yl]methyl}methanamine |
| 3164. | 1-(5-butyl-6-phenyl-3-pyrrolidin-1-ylpyridazin-4-yl)-N-(2,3-dihydro-1,4-benzodioxin- |
| | 6-ylmethyl)-N-(3-ethoxybenzyl)methanamine |
| 3165. | 1-(5-butyl-6-phenyl-3-piperidin-1-ylpyridazin-4-yl)-N-(2,3-dihydro-1,4-benzodioxin-6- |
| | ylmethyl)-N-(3-ethoxybenzyl)methanamine |
| 3166. | 1-(5-butyl-3-isopropoxy-6-phenylpyridazin-4-yl)-N-(2,3-dihydro-1,4-benzodioxin-6- |
| | ylmethyl)-N-(3-ethoxybenzyl)methanamine |
| 3167. | 1-(5-butyl-3-isobutoxy-6-phenylpyridazin-4-yl)-N-(2,3-dihydro-1,4-benzodioxin-6- |
| | ylmethyl)-N-(3-ethoxybenzyl)methanamine |
| 3168. | 4-[(5-butyl-4-{[(2,3-dihydro-1,4-benzodioxin-6-ylmethyl)(3- |
| | ethoxybenzyl)amino]methyl}-6-phenylpyridazin-3-yl)oxy]-2-methylbutan-2-ol |
| 3169. | N-[(5-butyl-3-chloro-6-phenylpyridazin-4-yl)methyl]-N-(2,3-dihydro-1,4-benzodioxin- |
| | 6-ylmethyl)butan-1-amine |
| 3170. | N-[(5-butyl-6-phenyl-3-piperidin-1-ylpyridazin-4-yl)methyl]-N-(2,3-dihydro-1,4- |
| | benzodioxin-6-ylmethyl)butan-1-amine |
| 3171. | N-[(5-butyl-3-isobutoxy-6-phenylpyridazin-4-yl)methyl]-N-(2,3-dihydro-1,4- |
| | benzodioxin-6-ylmethyl)butan-1-amine |
| 3172. | N,5-dibutyl-4-{[butyl(2,3-dihydro-1,4-benzodioxin-6-ylmethyl)amino]methyl}-6- |
| | phenylpyridazin-3-amine |
| 3173. | N-{[5-butyl-3-(4-methylpiperazin-1-yl)-6-phenylpyridazin-4-yl]methyl}-N-(2,3- |
| | dihydro-1,4-benzodioxin-6-ylmethyl)butan-1-amine |
| 3174. | N-{[5-butyl-3-(2-methoxyethoxy)-6-phenylpyridazin-4-yl]methyl}-N-(2,3-dihydro-1,4- |
| | benzodioxin-6-ylmethyl)butan-1-amine |
| 3175. | 1-(5-butyl-2-methoxy-6-phenylpyrimidin-4-yl)-N-(2,3-dihydro-1,4-benzodioxin-6- |
| | ylmethyl)-N-(3-ethoxybenzyl)methanamine |
| 3176. | N-{[5-butyl-3-(3,5-dimethylpiperidin-1-yl)-6-phenylpyridazin-4-yl]methyl}-N-(2,3- |
| | dihydro-1,4-benzodioxin-6-ylmethyl)butan-1-amine |
| 3177. | 5-butyl-4-{[butyl(2,3-dihydro-1,4-benzodioxin-6-ylmethyl)amino]methyl}-N-methyl-6- |
| | phenyl-N-propylpyridazin-3-amine |
| 3178. | 4-{[[(5-butyl-3-chloro-6-phenylpyridazin-4-yl)methyl](2,3-dihydro-1,4-benzodioxin-6- |
| | ylmethyl)amino]methyl}benzoic acid |
| 3179. | 4-{[[(5-butyl-3-isopropoxy-6-phenylpyridazin-4-yl)methyl](2,3-dihydro-1,4- |
| | benzodioxin-6-ylmethyl)amino]methyl}benzoic acid |
| 3180. | 4-{[[(5-butyl-3-isobutoxy-6-phenylpyridazin-4-yl)methyl](2,3-dihydro-1,4- |
| | benzodioxin-6-ylmethyl)amino]methyl}benzoic acid |
| 3181. | 5-butyl-4-{[(2,3-dihydro-1,4-benzodioxin-6-ylmethyl)(3-ethoxybenzyl)amino]methyl}- |
| | 6-phenylpyrimidin-2-ol |
| 3182. | 4-{[[(5-butyl-2-methoxy-6-phenylpyrimidin-4-yl)methyl](2,3-dihydro-1,4-benzodioxin- |
| | 6-ylmethyl)amino]methyl}benzoic acid |
| 3183. | 4-({[(5-butyl-3-chloro-6-phenylpyridazin-4-yl)methyl][4- |
| | (difluoromethoxy)benzyl]amino}methyl)benzoic acid |
| 3184. | 4-{[[(5-butyl-2-isopropoxy-6-phenylpyrimidin-4-yl)methyl](2,3-dihydro-1,4- |
| | benzodioxin-6-ylmethyl)amino]methyl}benzoic acid |
| 3185. | 4-{[[(5-butyl-2-isobutoxy-6-phenylpyrimidin-4-yl)methyl](2,3-dihydro-1,4- |
| | benzodioxin-6-ylmethyl)amino]methyl}benzoic acid |
| 3186. | 4-{[[(5-butyl-3-chloro-6-phenylpyridazin-4-yl)methyl](2,3-dihydro-1,4-benzodioxin-6- |
| | ylmethyl)amino]methyl}-3-fluorobenzoic acid |
| 3187. | 4-{[[(5-butyl-3-chloro-6-phenylpyridazin-4-yl)methyl](2,3-dihydro-1,4-benzodioxin-6- |
| | ylmethyl)amino]methyl}-3-chlorobenzoic acid |
| 3188. | 4-{[[(5-butyl-2-methyl-6-phenylpyrimidin-4-yl)methyl](2,3-dihydro-1,4-benzodioxin- |
| | 6-ylmethyl)amino]methyl}benzoic acid |
| 3189. | N-[(5-butyl-3-chloro-6-phenylpyridazin-4-yl)methyl]-N-(2-fluoro-4-methoxybenzyl)- |
| | 2,2-dimethylpropan-1-amine |
| 3190. | N-[(5-butyl-3-ethoxy-6-phenylpyridazin-4-yl)methyl]-N-(2-fluoro-4-methoxybenzyl)- |
| | 2,2-dimethylpropan-1-amine |
| 3191. | N-benzyl-N-(2-methylbenzyl)-2-(1H-pyrrol-1-yl)benzamide |
| 3192. | N-(2-methoxybenzyl)-N-(2-methylbenzyl)-2-(1H-pyrrol-1-yl)benzamide |
| 3193. | N-(2,2-diphenylethyl)-N-(2-methylbenzyl)-2-(1H-pyrrol-1-yl)benzamide |
| 3194. | N-(2,4-difluorobenzyl)-N-(2-methoxybenzyl)-2-(1H-pyrrol-1-yl)benzamide |
| 3195. | N-(2,4-difluorobenzyl)-N-(2,2-diphenylethyl)-2-(1H-pyrrol-1-yl)benzamide |
| 3196. | N-(2,6-dichlorobenzyl)-N-(2-methoxybenzyl)-2-(1H-pyrrol-1-yl)benzamide |
| 3197. | N-benzyl-N-(1,1′-biphenyl-2-ylmethyl)-2-(1H-pyrrol-1-yl)benzamide |
| 3198. | N-(1,1′-biphenyl-2-ylmethyl)-N-(2-methoxybenzyl)-2-(1H-pyrrol-1-yl)benzamide |
| 3199. | N-(1,1′-biphenyl-2-ylmethyl)-N-(2-phenylbutyl)-2-(1H-pyrrol-1-yl)benzamide |
| 3200. | N-(1,1′-biphenyl-2-ylmethyl)-N-(2,2-diphenylethyl)-2-(1H-pyrrol-1-yl)benzamide |
| 3201. | N-(1,1′-biphenyl-2-ylmethyl)-N-(cyclohexylmethyl)-2-(1H-pyrrol-1-yl)benzamide |
| 3202. | N-(2,4-dimethoxybenzyl)-N-(4-fluorobenzyl)-2-(1H-pyrrol-1-yl)benzamide |
| 3203. | N-(2-methoxybenzyl)-N-(4-methylbenzyl)-2-(1H-pyrrol-1-yl)benzamide |
| 3204. | N-(3-methoxybenzyl)-N-(4-methylbenzyl)-2-(1H-pyrrol-1-yl)benzamide |
| 3205. | N-(4-methoxybenzyl)-N-(4-methylbenzyl)-2-(1H-pyrrol-1-yl)benzamide |
| 3206. | N-(4-methylbenzyl)-2-(1H-pyrrol-1-yl)-N-[4-(trifluoromethoxy)benzyl]benzamide |
| 3207. | N-(2,4-dimethoxybenzyl)-N-(4-methylbenzyl)-2-(1H-pyrrol-1-yl)benzamide |
| 3208. | N-(3,5-dimethoxybenzyl)-N-(4-methylbenzyl)-2-(1H-pyrrol-1-yl)benzamide |
| 3209. | N-(4-chlorobenzyl)-N-(2-methoxybenzyl)-2-(1H-pyrrol-1-yl)benzamide |
| 3210. | N-(4-chlorobenzyl)-N-(3-methoxybenzyl)-2-(1H-pyrrol-1-yl)benzamide |
| 3211. | N-(4-chlorobenzyl)-N-(4-methoxybenzyl)-2-(1H-pyrrol-1-yl)benzamide |
| 3212. | N-(1,3-benzodioxol-5-ylmethyl)-N-(4-chlorobenzyl)-2-(1H-pyrrol-1-yl)benzamide |
| 3213. | N-(4-chlorobenzyl)-2-(1H-pyrrol-1-yl)-N-[4-(trifluoromethoxy)benzyl]benzamide |
| 3214. | N-(4-chlorobenzyl)-N-(2,4-dimethoxybenzyl)-2-(1H-pyrrol-1-yl)benzamide |
| 3215. | N-(2-methoxybenzyl)-2-(1H-pyrrol-1-yl)-N-[4-(trifluoromethyl) benzyl]benzamide |
| 3216. | N-(4-methoxybenzyl)-2-(1H-pyrrol-1-yl)-N-[4-(trifluoromethyl) benzyl]benzamide |
| 3217. | N-(2,4-dimethoxybenzyl)-2-(1H-pyrrol-1-yl)-N-[4-(trifluoromethyl) benzyl]benzamide |
| 3218. | N-(3,5-dimethoxybenzyl)-2-(1H-pyrrol-1-yl)-N-[4-(trifluoromethyl) benzyl]benzamide |
| 3219. | N-(2-methoxybenzyl)-2-(1H-pyrrol-1-yl)-N-[2,4-(difluoro) benzyl]benzamide |
| 3220. | N-(2,4-difluorobenzyl)-N-(4-methoxybenzyl)-2-(1H-pyrrol-1-yl)benzamide |
| 3221. | N-(2,4-difluorobenzyl)-N-(2,4-dimethoxybenzyl)-2-(1H-pyrrol-1-yl)benzamide |
| 3222. | N-(2,4-dichlorobenzyl)-N-(2-methoxybenzyl)-2-(1H-pyrrol-1-yl)benzamide |
| 3223. | N-(2,4-dichlorobenzyl)-N-(3-methoxybenzyl)-2-(1H-pyrrol-1-yl)benzamide |
| 3224. | N-(2,4-dichlorobenzyl)-N-(4-methoxybenzyl)-2-(1H-pyrrol-1-yl)benzamide |
| 3225. | N-(1,3-benzodioxol-5-ylmethyl)-N-(2,4-dichlorobenzyl)-2-(1H-pyrrol-1-yl)benzamide |
| 3226. | N-(2,4-dichlorobenzyl)-2-(1H-pyrrol-1-yl)-N-[4-(trifluoromethoxy)benzyl]benzamide |
| 3227. | N-(2,4-dichlorobenzyl)-N-(2,4-dimethoxybenzyl)-2-(1H-pyrrol-1-yl)benzamide |
| 3228. | N-(2,4-dichlorobenzyl)-N-(3,5-dimethoxybenzyl)-2-(1H-pyrrol-1-yl)benzamide |
| 3229. | N-(2-chloro-4-fluorobenzyl)-N-(3-methoxybenzyl)-2-(1H-pyrrol-1-yl)benzamide |
| 3230. | N-(2-chloro-4-fluorobenzyl)-N-(4-methoxybenzyl)-2-(1H-pyrrol-1-yl)benzamide |
| 3231. | N-(1,3-benzodioxol-5-ylmethyl)-N-(2-chloro-4-fluorobenzyl)-2-(1H-pyrrol-1- |
| | yl)benzamide |
| 3232. | N-(2-chloro-4-fluorobenzyl)-N-(2,4-dimethoxybenzyl)-2-(1H-pyrrol-1-yl)benzamide |
| 3233. | N-(2-chloro-4-fluorobenzyl)-N-(3,5-dimethoxybenzyl)-2-(1H-pyrrol-1-yl)benzamide |
| 3234. | N-(2,3-difluorobenzyl)-N-(2,4-dimethoxybenzyl)-2-(1H-pyrrol-1-yl)benzamide |
| 3235. | N-(3-chloro-2-fluorobenzyl)-N-(2,4-dimethoxybenzyl)-2-(1H-pyrrol-1-yl)benzamide |
| 3236. | N-(3-chloro-2-fluorobenzyl)-N-(3,5-dimethoxybenzyl)-2-(1H-pyrrol-1-yl)benzamide |
| 3237. | N-[(5-chlorothien-2-yl)methyl]-N-(4-methoxybenzyl)-2-(1H-pyrrol-1-yl)benzamide |
| 3238. | N-[(5-chlorothien-2-yl)methyl]-N-(2,4-dimethoxybenzyl)-2-(1H-pyrrol-1-yl)benzamide |
| 3239. | N-[(5-chlorothien-2-yl)methyl]-N-(3,5-dimethoxybenzyl)-2-(1H-pyrrol-1-yl)benzamide |
| 3240. | N-(2,5-difluorobenzyl)-N-(2,4-dimethoxybenzyl)-2-(1H-pyrrol-1-yl)benzamide |
| 3241. | N-(2,4-difluorobenzyl)-N-[2-(2-fluorophenyl)ethyl]-2-(1H-pyrrol-1-yl)benzamide |
| 3242. | N-(indan-2-yl)-N-(3-methylbenzyl)-2-(1H-pyrrol-1-yl)benzamide |
| 3243. | N-(indan-2-yl)-N-(4-methylbenzyl)-2-(1H-pyrrol-1-yl)benzamide |
| 3244. | N-(indan-1-yl)-2-(1H-pyrrol-1-yl)-N-[4-(trifluoromethyl)benzyl]benzamide |
| 3245. | N-(2,3-dihydro-1H-inden-1-yl)-N-(4-methylbenzyl)-2-(1H-pyrrol-1-yl)benzamide |
| 3246. | N-Indan-2-yl-N-(4-methyl-benzyl)-2-pyrrol-1-yl-benzamide |
| 3247. | N-(4-chlorobenzyl)-N-(2,3-dihydro-1H-inden-1-yl)-2-(1H-pyrrol-1-yl)benzamide |
| 3248. | N-(4-chlorobenzyl)-N-(indan-2-yl)-2-(1H-pyrrol-1-yl)benzamide |
| 3249. | N-(indan-2-yl)-2-(1H-pyrrol-1-yl)-N-[4-(trifluoromethyl)benzyl]benzamide |
| 3250. | N-(2,3-dihydro-1H-inden-1-yl)-N-(3,5-dimethylbenzyl)-2-(1H-pyrrol-1-yl)benzamide |
| 3251. | N-(indan-2-yl)-N-(3,5-dimethylbenzyl)-2-(1H-pyrrol-1-yl)benzamide |
| 3252. | N-(indan-2-yl)-N-(1-naphthylmethyl)-2-(1H-pyrrol-1-yl)benzamide |
| 3253. | N-(2,3-dihydro-1H-inden-1-yl)-N-(2-naphthylmethyl)-2-(1H-pyrrol-1-yl)benzamide |
| 3254. | N-(indan-2-yl)-N-(2-naphthylmethyl)-2-(1H-pyrrol-1-yl)benzamide |

The foregoing description is illustrative thereof, and it will be understood that variations and modifications can be effected without departing from the scope or spirit of the invention as set forth in the following claims.

What is claimed is:

1. A composition comprising a therapeutically effective amount of at least one C5a antagonist and a therapeutically effective amount of at least one C5a receptor-inactive therapeutic agent, wherein the C5a antagonist is a compound of the formula: 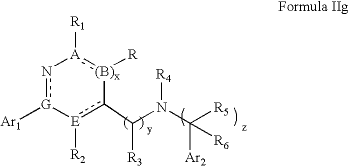
or a pharmaceutically acceptable salts thereof, wherein 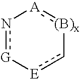
is: i) a 5-membered heteroaryl ring system, in which x is 0; A is carbon, nitrogen, oxygen, or sulfur; and E and G are independently carbon or nitrogen, provided that the 5-membered heteroaryl ring system does not contain more than 3 heteroatoms or more than 1 oxygen or sulfur atom; or
ii) a 6-membered heteroaryl ring system, in which x is 1; A, B, E, and G are independently chosen from carbon and nitrogen, provided that the 6-membered heteroaryl ring system does not contain more than 3 nitrogen atoms.
R and R₁ independently represent:
i) hydrogen, hydroxy, halogen, amino, cyano, nitro, —CHO, —CONH₂, C₁-C₆haloalkyl, or C₁-C₆ haloalkoxy;
ii) C₁-C₆alkyl, C₁-C₆alkenyl, C₁-C₆alkynyl, C₁-C₆ alkoxy, C₃-C₇cycloalkyl, (C₃-C₇cycloalkyl)C₁-C₄alkyl, mono- or di-C₁-C₆alkylamino, mono- or di-C₁-C₆alkyl aminoC₁-C₆alkyl, mono- or di-C₁-C₆alkylcarboxamide, C₁-C₆alkoxycarbonyl, —NHSOₙC₁-C₆alkyl, —SOₙN(C₁-C₆alkyl) (C₁-C₆alkyl), or phenyl-SOₙ—, each of which is optionally substituted, wherein n is 0, 1 or 2; or
iii) naphthyl, phenyl, phenylC₁-C₄carbhydryl, 5- or 6-membered heteroaryl, or 5- or 6-membered heteroarylC₁-C₄carbhydryl, each of which is optionally substituted;
If E is nitrogen, R₂ is chosen from C₁-C₇alkyl, C₂-C₇alkenyl, C₂-C₇ alkynyl, C₃-C₇cycloalkyl(C₁-C₄alkyl), benzyl and C₁-C₆haloalkyl;
If E is carbon, R₂ is chosen from halogen, hydroxy, C₁-C₇alkyl, C₂-C₇alkenyl, C₂-C₇alkynyl, C₁-C₇alkoxy, C₁-C₇alkylamino, C₃-C₇cycloalkyl(C₁-C₄alkyl), benzyl, C₁-C₆haloalkyl, and C₁-C₆haloalkoxy;
R₃ is hydrogen, C₁-C₆alkyl, C₂-C₆alkenyl, hydroxyC[-C₆alkyl, C₁-C₆haloalkyl, C₃-C₇cycloalkyl, (C₃-C₇cycloalkyl)C₁-C₄alkyl, or phenyl(C₁-C₄alkyl); or when x is 0, R₁ and R₃ may be joined to form an optionally substituted (C₃-C₇)cycloalkyl;
R₄ is i) hydrogen;
ii) C₁-C₆alkyl, C₂-C₆alkenyl, C₂-C₆alkynyl, C₃-C₇cycloalkyl, C₃-C₇cycloalkenyl, (C₃-C₇cycloalkyl)C₁-C₄alkyl, (C₃-C₇cycloalkenyl)C₁-C₄alkyl, or hexahydro-1,3-benzodioxolylmethyl, each of which is optionally substituted;
iii) optionally substituted arylC₁-C₄alkyl having 1 or 2 fused or pendant rings;
iv) optionally substituted arylC₁-C₄alkyl, wherein the aryl portion is fused to a 5- to 7-membered saturated or partially unsaturated ring having 0, 1, or 2 ring atoms independently chosen from N, O, and S with remaining ring atoms being carbon;
v) optionally substituted heterocycloalkyl(C₀C₄alkyl);
vi) optionally substituted heteroarylC₁-C₂alkyl having from 1 to 2 fused or pendant rings, from 5 to 7 members in each ring, and in at least one ring from 1 to 3 heteroatoms independently selected from N, O, and S; or
vii) optionally substituted saturated or partially unsaturated heterocyclic(C₀C₄alkyl) having from 4 to 7 ring members, 1 or 2 of which ring members are N, S or O, with remaining ring members being carbon;
R₅ and R₆ are independently chosen from hydrogen and C₁-C₆alkyl;
z is 1, 2, or 3;
Ar₁ is i) optionally substituted aryl having 1 or 2 fused or pendant rings;
ii) optionally substituted phenyl fused to a 5- to 7-membered saturated or partially unsaturated ring having 0, 1, or 2 ring atoms independently chosen from N, O, and S with remaining ring atoms being carbon; or
iii) optionally substituted heteroaryl having 1 or 2 fused or pendant rings, from 5 to 7 members in each ring, and in at least one ring from 1 to 3 heteroatoms independently selected from N, O, and S;
Ar₂ is i) optionally substituted C₃-C₇cycloalkyl, C₃-C₇cycloalkyl(C₁-C₄alkyl), C₃-C₇cycloalkenyl, C₃-C₇cycloalkenyl(C₁-C₄alkyl), or hexahydro-1,3-benzodioxolyl;
ii) optionally substituted aryl having 1 or 2 fused or pendant rings;
iii) optionally substituted phenyl fused to a 5- to 7-membered saturated or partially unsaturated ring having 0, 1, or 2 ring atoms independently chosen from N, O, and S with remaining ring atoms being carbon; or
iv) optionally substituted heteroaryl having 1 or 2 fused or pendant rings, from 5 to 7 members in each ring, and in at least one ring from 1 to 3 heteroatoms independently selected from N, O, and S; and
y is 1 or 2.

2. A composition according to claim 1, wherein x is 0, A and G are carbon, and E is nitrogen.

3. A composition according to claim 2, wherein:
Ar₁ is phenyl, substituted with from 0 to 3 substituents independently chosen from hydroxy, cyano, halogen, methyl, ethyl, methoxy, and ethoxy; and
R₁ is phenyl substituted with from 0 to 3 substituents independently chosen from halogen, hydroxy, nitro, methyl, ethyl, methoxy, ethoxy, trifluoromethyl, difluoromethyl, trifluoromethoxy, difluoromethoxy, —CONH₂, —OC(═O)CH₃, —COOH, methylthio, ethylthio, and —SO₂CH₃.

4. A composition according to claim 3, wherein z is 1, R₂ is C₃-C₅alkyl and R₃ is hydrogen or methyl.

5. A composition according to claim 4, wherein R₄ is:
i) 1,3-benzodioxol-5-ylmethyl or 2,3-dihydro-1,4-benzodioxin-6-ylmethyl, each of which is substituted with from 0 to 3 substituents independently chosen from halogen, methyl and methoxy; or
ii) benzyl substituted with from 0 to 3 substituents independently chosen from halogen, hydroxy, nitro, methyl, ethyl, methoxy, ethoxy, trifluoromethyl, difluoromethyl, pentafluoroethyl, —CF₂CHF₂, trifluoromethoxy, difluoromethoxy, pentafluoroethoxy, —OCF₂CHF₂, —CONH₂, —C(═O)OCH₃, —OC(═O)CH₃, —COOH, methylthio, ethylthio, —SO₂NH₂, and —SO₂CH₃.

6. A composition according to claim 5, wherein
R₅ is hydrogen or methyl;
R₆ is hydrogen; and
Ar₂ is: i) benzo[1,3]dioxole or 2,3-dihydro-benzo[1,4]dioxine, each of which is substituted with from 0 to 3 substituents independently chosen from halogen, methyl and methoxy; or
ii) phenyl substituted with from 0 to 3 substituents independently chosen from halogen, hydroxy, nitro, methyl, ethyl, methoxy, ethoxy, trifluoromethyl, difluoromethyl, pentafluoroethyl, —CF₂CHF₂, trifluoromethoxy, difluoromethoxy, pentafluoroethoxy, —OCF₂CHF₂, —CONH₂, —C(═O)OCH₃, —OC(═O)CH₃, —COOH, methylthio, ethylthio, —SO₂NH₂, and —SO₂CH₃

7. A composition according to claim 1, wherein the C5a antagonist exhibits C5a antagonist activity with an IC₅₀ of less than 100 nM in a standard assay for C5a antagonist activity.

8. A composition according to claim 1, wherein C5a receptor-inactive therapeutic agent is an NSAID.

9. A composition according to claim 1, wherein the C5a receptor-inactive therapeutic agent is a cyclo-oxygenase enzyme inhibitor.

10. A composition according to claim 1, wherein the C5a receptor-inactive therapeutic agent is an inhibitor of the cyclo-oxygenase 2 enzyme.

11. A composition according to claim 1, wherein the C5a receptor-inactive therapeutic agent is a gold compound or a salicylate.

12. A composition according to claim 1, wherein C5a receptor-inactive therapeutic agent is a steroid.

13. A composition according to claim 12, wherein C5a receptor-inactive therapeutic agent is a corticosteroid.

14. A composition according to claim 1, wherein C5a receptor-inactive therapeutic agent is methotrexate or leflunomide.

15. A composition according to claim 1, wherein C5a receptor-inactive therapeutic agent is a TNF antagonist.

16. A composition according to claim 1, wherein the C5a receptor-inactive therapeutic agent is a cholesterol lowering agent.

17. A composition according to claim 16, wherein the cholesterol lowering agent is an HMG-CoA reductase inhibitor.

18. A composition according to claim 1, wherein the C5a receptor-inactive therapeutic agent is a platelet aggregation inhibitor.

19. A composition according to claim 1, wherein the C5a receptor-inactive therapeutic agent is an anti-hypertensive agent.

20. A composition according to claim 19, wherein the antihypertensive agent is an adrenergic receptor stimulator.

21. A composition according to claim 19, wherein the antihypertensive agent is an alpha/beta adrenergic receptor antagonist, beta adrenergic receptor antagonist, ACE inhibitor, angiotensin II receptor antagonist, calcium channel blocker, diuretic, or periphereal vasodilator.

22. A composition comprising a therapeutically effective amount of at least one C5a antagonist and a therapeutically effective amount of at least one C5a receptor-inactive therapeutic agent, wherein the C5a antagonist is selected from:
1-(1-Butyl)-2-(2-methoxyphenyl)-5-(N,N-di[3,4-methylenedioxyphenylmethyl]) aminomethylimidazole;
1-(1-Butyl)-2-(2-methoxyphenyl)-5-(N-[4-dimethylaminophenylmethyl]-N-phenylmethyl) aminomethylimidazole; 1-(1-Butyl)-2-(2-methylphenyl)-5-(N-[3,4-methylenedioxyphenylmethyl]-N-phenylmethyl) aminomethylimidazole;
1-(1-Butyl)-2-(4-fluorophenyl)-5-(N,N-di[3,4-methylenedioxyphenylmethyl])amino-methylimidazole;
1-(1-Butyl)-2-(2-methylphenyl)-5-(N,N-di[3,4-methylenedioxyphenylmethyl])amino-methylimidazole;
1-(1-Butyl)-2-(3-fluorophenyl)-5-(N-[naphth-2-ylmethyl]-N-phenylmethyl)amino methylimidazole;
1-(1-Butyl)-2-(3-fluorophenyl)-5-(N-[3,4-methylenedioxyphenylmethyl]-N-phenylmethyl) aminomethylimidazole;
1-(1-Butyl)-2-(3-fluorophenyl)-5-(N,N-di[3,4-methylenedioxyphenylmethyl])amino-methylimidazole;
1-(1-Butyl)-2-(3-methoxyphenyl)-5-(N-[3,4-methylenedioxyphenylmethyl]-N-phenylmethyl)-aminomethylimidazole;
1-(1-Butyl)-2-phenyl-5-{1-(N-[3,4-methylenedioxyphenylmethyl]-N-phenylmethyl)amino}ethylimidazole;
1-(1-Pentyl)-2-phenyl-5-(N-[indol-5-ylmethyl]-N-phenylmethyl) aminomethylimidazole;
Bis-benzo[1,3]dioxol-5-ylmethyl-(3-butyl-2,5-diphenyl-3H-imidazol-4-ylmethyl)amine;
Benzo[1,3]dioxol-5-ylmethyl-benzyl-[3-butyl-5-(4-methoxy-phenyl)-2-phenyl-3H-imdiazol-4-ylmethyl]-amine;
4-({Benzyl-[1-(3-butyl-2,5-diphenyl-3H-imidazol-4-yl)-ethyl)-amino}-methyl)benzamide;
4-{[Benzyl-(3-butyl-2,5-diphenyl-3H-imidazol-4-ylmethyl)-amino]-methyl}3-chloro-phenol;
4-({[1-(3-Butyl-2-phenyl-3H-imidazol-4-yl)-pentyl]-cyclohexymethyl-amino}-methyl)-phenol;
4-{[Benzyl-(3-butyl-2,5-diphenyl-3H-imidazol-4-ylmethyl)-amino]-methyl}benzamide;
1-(1-Propyl)-2-phenyl-5-(N-[indol-5-ylmethyl]-N-phenylmethyl) aminomethylimidazole;
1-(1-Butyl)-2-phenyl-5-(N-[1-(S)-phenylethyl]-N-phenylmethyl)aminomethylimidazole;
1-(1-Butyl)-2-phenyl-5-(N-[1-(R)-phenylethyl]-N-phenylmethyl)aminomethylimidazole 1-(1-Butyl)-2-phenyl-5-(N-[3,4-methylenedioxyphenylmethyl]-N-[3,4-dichlorophenyl]methyl)aminomethylimidazole;
1-(1-Butyl)-2-phenyl-5-(N,N-di[3,4-methylenedioxyphenylmethyl]) aminomethylimidazole;
1-(1-Butyl)-2-phenyl-5-(N-[3,4-methylenedioxyphenylmethyl]-N-[3,4-methoxyphenylmethyl])-aminomethylimidazole;
1-(1-Butyl)-2-phenyl-5-(N-[3,4-methylenedioxyphenylmethyl]-N-[4-{1-propyl}phenylmethyl])aminomethylimidazole;
1-(1-Butyl)-2-phenyl-5-(N-[3,4-methylenedioxyphenylmethyl]-N-[3,4-dichlorophenylethyl])aminomethylimidazole;
1-(1-Butyl)-2-phenyl-5-(N-[3,4-methylenedioxyphenyl]methyl-N-[4-nitrophenylmethyl])aminomethylimidazole;
1-(1-Butyl)-2-phenyl-5-(N-[3,4-methylenedioxyphenylmethyl]-N-[4-{1-propyloxy}phenylmethyl])aminomethylimidazole;
1-(1-Butyl)-2-phenyl-5-(N-[3,4-methylenedioxyphenylmethyl]-N-[quinol-6-ylmethyl])-aminomethylimidazole;
1-(1-Butyl)-2-phenyl-5-(N-[3,4-methylenedioxyphenylmethyl]-N-[2,3-dichlorophenylmethyl])-aminomethylimidazole;
1-(1-Butyl)-2-phenyl-5-(N-[3,4-methylenedioxyphenylmethyl]-N-[3,4-dimethylphenylmethyl])-aminomethylimidazole;
1-(1-Butyl)-2-phenyl-5-(N-[3,4-methylenedioxyphenyl]methyl-N-[indan-2-yl])-aminomethylimidazole;
1-(1-Butyl)-2-phenyl-5-(N-[3,4-methylenedioxyphenylmethyl]-N-[2-phenylethyl])aminomethylimidazole;
1-(1-Propyl)-2-phenyl-5-(N-[1,4-benzodioxan-6-ylmethyl]-N-phenylmethyl)aminomethylimidazole;
1-(1-Butyl)-2-phenyl-5-(N-[3,4-methylenedioxyphenylmethyl]-N-phenylmethyl)aminomethylimidazole;
1-(1-Butyl)-2-phenyl-5-(N-[3,4-methylenedioxyphenylmethyl]-N-ethyl)aminomethylimidazole;
1-(1-Butyl)-2-phenyl-5-(N-[3,4-methylenedioxyphenylmethyl]-N-[1-propyl])aminomethylimidazole; 1-(1-Butyl)-2-phenyl-5-(N-[3,4-methylenedioxyphenylmethyl]-N-[1-butyl])aminomethylimidazole;
1-(1-Butyl)-2-phenyl-5-(N-[3,4-methylenedioxyphenylmethyl]-N-cycloheptylmethyl)aminomethylimidazole;
1-(1-Butyl)-2-phenyl-5-(N-[3,4-methylenedioxyphenylmethyl]-N-isobutyl)aminomethylimidazole;
1-(1-Butyl)-2-phenyl-5-(N-[3,4-methylenedioxyphenylmethyl]-N-[2-cyclopentylethyl])aminomethylimidazole;
1-(1-Butyl)-2-phenyl-5-(N-[3,4-methylenedioxyphenylmethyl]-N-[3-cyclopentylpropyl])amino-methylimidazole;
1-(1-Butyl)-2-phenyl-5-(N-[3,4-methylenedioxyphenylmethyl]-N-[1-n-octyl])aminomethylimidazole;
1-(1-Butyl)-2-phenyl-5-(N-[3,4-methylenedioxyphenylmethyl]-N-cyclopropylmethyl)aminomethylimidazole;
1-(1-Butyl)-2-phenyl-5-(N-[3,4-methylenedioxyphenylmethyl]-N-cyclopentylmethyl)aminomethylimidazole; 1-(1-Butyl)-2-phenyl-5-(N-[3,4-methylenedioxyphenylmethyl]-N-cyclohexylmethyl)aminomethylimidazole;
1-(1-Butyl)-2-phenyl-5-(N-[3,4-methylenedioxyphenylmethyl]-N-[t-amyl])aminomethylimidazole;
1-(1-Butyl)-2-phenyl-5-(N-[3,4-methylenedioxyphenylmethyl]-N-[1-{3-methyl}butyl)]aminomethylimidazole;
1-(1-Butyl)-2-phenyl-5-(N-[3,4-methylenedioxyphenylmethyl]-N-[1-{2,2-dimethyl}butyl]) aminomethylimidazole;
1-(1-Butyl)-2-phenyl-5-(N-[3,4-methylenedioxyphenylmethyl]-N-methyl)aminomethylimidazole;
1-(1-Butyl)-2-phenyl-5-(N-[3,4-methylenedioxyphenylmethyl]-N-[2-thiophenylmethyl])aminomethylimidazole;
1-(1-Butyl)-2-phenyl-5-(N-[3,4-methylenedioxyphenylmethyl]-N-[indol-5-ylmethyl])aminomethylimidazole;
1-(1-Butyl)-2-phenyl-5-(N-[3,4-methylenedioxyphenylmethyl]-N-[{1-methylindol-5-yl}methyl])aminomethylimidazole;
1-(1-Butyl)-2-phenyl-5-(N-[3,4-methylenedioxyphenyl]methyl-N-[4-hydroxy-2-chlorophenyl]-methyl)aminomethylimidazole;
1-(1-Butyl)-2-(3-fluorophenyl)-5-(1-[N-{2-chloro-4-hydroxyphenyl}methyl-N-phenylmethyl]) aminoethylimidazole;
1-(1-Butyl)-2-phenyl-5-(N-[3,4-methylenedioxyphenyl]methyl-N-[2,3-dihydrobenzo[b]furan-5-yl]methyl)aminomethylimidazole;
1-Butyl-2-(4-fluorophenyl)-5-(1-[N-{3,4-methylenedioxyphenyl}methyl-N-phenylmethyl]-amino)ethylimidazole;
1-(1-Butyl)-2-(2-thienyl)-5-(N-[3,4-methylenedioxyphenyl]methyl-N-phenylmethyl]aminomethylimidazole;
1-(1-Butyl)-2-phenyl-5-(N-[3,4,5-trimethoxyphenylmethyl]-N-phenylmethyl)aminomethylimidazole;
1-(1-Butyl)-2-phenyl-5-(N-phenylmethyl-N-[3,4-dimethoxyphenylmethyl])aminomethylimidazole;
1-(1-Butyl)-2-phenyl-5-(N-[4-dimethylaminophenylmethyl]-N-phenylmethyl)aminomethylimidazole;
1-(1-Butyl)-2-phenyl-5-(N-[4-methylaminophenylmethyl]-N-phenylmethyl)aminomethylimidazole;
1-(1-Butyl)-2-phenyl-5-(N-[3-methyl-4-aminophenylmethyl]-N-phenylmethyl)aminomethylimidazole);
1-(1-Butyl)-2-phenyl-5-(N-[2,3-dichlorophenylmethyl]-N-phenylmethyl)aminomethylimidazole;
1-(1-Butyl)-2-phenyl-5-(N-[3,4-dichlorophenylmethyl]-N-phenylmethyl)aminomethylimidazole;
1-(1-Butyl)-2-phenyl-5-(N-[3,4-difluorophenylmethyl]-N-phenylmethyl)aminomethylimidazole;
1-(1-Butyl)-2-phenyl-5-(N-(benzo[b]thiophen-5-ylmethyl)-N-phenylmethyl)aminomethylimidazole; and
1-(1-Butyl)-2-phenyl-5-(N-[4-ethoxyphenylmethyl]-N-phenylmethyl)aminomethylimidazole.

23. A composition comprising a therapeutically effective amount of at least one C5a antagonist and a therapeutically effective amount of at least one C5a receptor-inactive therapeutic agent, wherein the C5a antagonist is selected from:
N-benzyl-N-(2,3-dihydro-1H-indan-2-yl)-2-[1-(3-methylphenyl)-3,4-dihydroisoquinolin-2(1H)-yl]acetamide;
N-benzyl-N-(2,3-dihydro-1H-indan-2-yl)-2-[1-(4-methylphenyl)-3,4-dihydroisoquinolin-2(1H)-yl]acetamide;
N-benzyl-N-(2,3-dihydro-1H-indan-2-yl)-2-[1-(4-methoxyphenyl)-3,4-dihydroisoquinolin-2(1H)-yl]acetamide;
N-[(5-bromo-2-phenyl-1,3-thiazol-4-yl)methyl]-N-isopentyl-2-(1H-pyrrol-1-yl)benzamide;
N-butyl-N-[(5-chloro-2-phenyl-1,3-thiazol-4-yl)methyl]-2-(1H-pyrrol-1-yl)benzamide;
N-[(5-chloro-2-phenyl-1,3-thiazol-4-yl)methyl]-N-isobutyl-2-(1H-pyrrol-1-yl)benzamide;
N-[(5-chloro-2-phenyl-1,3-thiazol-4-yl)methyl]-N-isopentyl-2-(1H-pyrrol-1-yl)benzamide;
N-[(5-chloro-2-phenyl-1,3-thiazol-4-yl)methyl]-N-cyclopentyl-2-(1H-pyrrol-1-yl)benzamide;
8-bromo-N-[(2-phenylquinolin-4-yl)methyl]-N-propyl-1-naphthamide;
N-(2-chlorobenzyl)-N-(2-phenylethyl)-9H-fluorene-4-carboxamide;
2-(2-iodophenyl)-N-[(2-phenylquinolin-4-yl)methyl]-N-propylacetamide;
2-(2-phenoxyphenyl)-N-[(2-phenylquinolin-4-yl)methyl]-N-propylacetamide;
2-(2-methoxyphenyl)-N-[(2-phenylquinolin-4-yl)methyl]-N-propylacetamide;
2-(2-bromophenyl)-N-[(2-phenylquinolin-4-yl)methyl]-N-propylacetamide;
N-benzyl-N-[2-(2-chlorophenyl)ethyl]-9H-fluorene-4-carboxamide;
N-benzyl-N-(3-phenylbutyl)-9H-fluorene-4-carboxamide;
N-benzyl-N-[2-(4-chlorophenyl)ethyl]-9H-fluorene-4-carboxamide;
N-benzyl-N-(2,3-dihydro-1H-indan-2-yl)-1,1′-biphenyl-2-carboxamide;
N-benzyl-N-(2,3-dihydro-1H-indan-2-yl)-9H-fluorene-4-carboxamide;
N-benzyl-N-[2-(4-fluorophenyl)ethyl]-9H-fluorene-4-carboxamide;
N-benzyl-N-[2-(4-methoxyphenyl)ethyl]-9H-fluorene-4-carboxamide;
N-benzyl-N-[2-(4-methoxyphenyl)-1-methylethyl]-9H-fluorene-4-carboxamide;
2-chloro-N-{[1-(2,3-dihydro-1H-indan-2-yl)-2-phenyl-1H-imidazol-5-yl]methyl}-N-(4-methoxybenzyl)benzamide;
N-{[1-(2,3-dihydro-1H-indan-2-yl)-2-phenyl-1H-imidazol-5-yl]methyl}-2,5-difluoro-N-(4-methoxybenzyl)benzamide;
N-(cyclohexylmethyl)-N-{[1-(2,3-dihydro-1H-indan-2-yl)-2-phenyl-1H-imidazol-5-yl]methyl}-2,5-difluorobenzamide;
5-chloro-N{[1-(2,3-dihydro-1H-indan-2-yl)-2-phenyl-1H-imidazol-5-yl]methyl}-2-methoxy-N-(4-methoxybenzyl)benzamide;
5-chloro-N-(cyclohexylmethyl)-N-{[1-(2,3-dihydro-1H-indan-2-yl)-2-phenyl-1H-imidazol-5-yl]methyl}-2-methoxybenzamide;
N-{[1-(2,3-dihydro-1H-indan-2-yl)-2-phenyl-1H-imidazol-5-yl]methyl}-N-(4-methoxybenzyl)-2-(trifluoromethyl)benzamide;
N-benzyl-2,5-dichloro-N-{[1-(2,3-dihydro-1H-indan-2-yl)-2-phenyl-1H-imidazol-5-yl]methyl}benzamide;
2,5-dichloro-N-{[1-(2,3-dihydro-1H-indan-2-yl)-2-phenyl-1H-imidazol-5-yl]methyl}-N-(4-methoxybenzyl)benzamide;
2,5-dichloro-N-(cyclohexylmethyl)-N-{[1-(2,3-dihydro-1H-indan-2-yl)-2-phenyl-1H-imidazol-5-yl]methyl}benzamide;
2-bromo-N-{[1-(2,3-dihydro-1H-indan-2-yl)-2-phenyl-1H-imidazol-5-yl]methyl}-N-(4-methoxybenzyl)benzamide;
N-{[1-(2,3-dihydro-1H-indan-2-yl)-2-phenyl-1H-imidazol-5-yl]methyl)}-N-(4-methoxybenzyl)-2-(2-phenylethyl)benzamide;
N-{[1-(2,3-dihydro-1H-indan-2-yl)-2-phenyl-1H-imidazol-5-yl]methyl}-2-iodo-N-(4-methoxybenzyl)benzamide;
3-chloro-N-{[1-(2,3-dihydro-1H-indan-2-yl)-2-phenyl-1H-imidazol-5-yl]methyl}-2,6-dimethoxy-N-(4-methoxybenzyl)benzamide; and
2-bromo-N-{[1-(2,3-dihydro-1H-indan-2-yl)-2-phenyl-1H-imidazol-5-yl]methyl}-5-methoxy-N-(4-methoxybenzyl)benzamide.

24. A composition comprising a therapeutically effective amount of at least one C5a antagonist and a therapeutically effective amount of at least one C5a receptor-inactive therapeutic agent, wherein the C5a antagonist is selected from:
Ethyl 4-(3-{butyl [(4-chloro-2-phenyl-1-propyl-1H-imidazol-5-yl)methyl]amino}propyl)benzoate;
Ethyl 4-[3-(butyl {[1-butyl-4-chloro-2-(2-methylphenyl)-1H-imidazol-5-yl]methyl}amino)propyl]benzoate;
4-{3-[[(1-Butyl-4-chloro-2-phenyl-1H-imidazol-5-yl)methyl](2,3-dihydro-1,4-benzodioxin-6-ylmethyl)amino]propyl}benzoic acid;
4-[3-(Butyl {[1-butyl-4-chloro-2-(2-methylphenyl)-1H-imidazol-5-yl]methyl}amino)propyl]benzoic acid;
methyl (4-{[[(1-butyl-4-chloro-2-phenyl-1H-imidazol-5-yl)methyl](2,3-dihydro-1,4-benzodioxin-6-ylmethyl)amino]methyl}phenyl)acetate;
methyl 2-(4-{[[(1-butyl-4-chloro-2-phenyl-1H-imidazol-5-yl)methyl](2,3-dihydro-1,4-benzodioxin-6-ylmethyl)amino]methyl}phenyl)propanoate;
methyl 2-(4-{[[(1-butyl-4-chloro-2-phenyl-1H-imidazol-5-yl)methyl](2,3-dihydro-1,4-benzodioxin-6-ylmethyl)amino]methyl}phenyl)-2-methylpropanoate;
methyl (3-{[[(1-butyl-4-chloro-2-phenyl-1H-imidazol-5-yl)methyl](2,3-dihydro-1,4-benzodioxin-6-ylmethyl)amino]methyl}phenyl)acetate;
(4-{[[(1-butyl-4-chloro-2-phenyl-1H-imidazol-5-yl)methyl](2,3-dihydro-1,4-benzodioxin-6-ylmethyl)amino]methyl}phenyl)acetic acid;
2-(4-{[[(1-butyl-4-chloro-2-phenyl-1H-imidazol-5-yl)methyl](2,3-dihydro-1,4-benzodioxin-6-ylmethyl)amino]methyl}phenyl)propanoic acid;
2-(4-{[[(1-butyl-4-chloro-2-phenyl-1H-imidazol-5-yl)methyl](2,3-dihydro-1,4-benzodioxin-6-ylmethyl)amino]methyl}phenyl)-2-methylpropanoic acid;
(3-{[[(1-butyl-4-chloro-2-phenyl-1H-imidazol-5-yl)methyl](2,3-dihydro-1,4-benzodioxin-6-ylmethyl)amino]methyl}phenyl)acetic acid;
N-{[1-butyl-4-chloro-2-(2-methylphenyl)-1H-imidazol-5-yl]methyl}-N-(2,3-dihydro-1,4-benzodioxin-6-ylmethyl)butan-1-amine;
4-[(butyl {[1-butyl-4-chloro-2-(2-methylphenyl)-1H-imidazol-5-yl]methyl}amino)methyl]benzenesulfonamide;
4-{[{[1-butyl-4-chloro-2-(2-methylphenyl)-1H-imidazol-5-yl]methyl}(isopentyl)amino]methyl}benzenesulfonamide;
4-{[[(1-butyl-4-chloro-2-phenyl-1H-imidazol-5-yl)methyl](2,3-dihydro-1,4-benzodioxin-6-ylmethyl)amino]methyl}benzenesulfonamide;
N-{[1-butyl-4-chloro-2-(2-methylphenyl)-1H-imidazol-5-yl]methyl}-N-[(3-chloro-1H-indol-5-yl)methyl]-3-methylbutan-1-amine;
5-{[{[1-butyl-4-chloro-2-(2-methylphenyl)-1H-imidazol-5-yl]methyl}(isopentyl)amino]methyl}-1H-indole-3-carbonitrile;
4-{[{[1-butyl-4-chloro-2-(2-methylphenyl)-1H-imidazol-5-yl]methyl}(2,3-dihydro-1,4-benzodioxin-6-ylmethyl)amino]methyl}benzenesulfonamide;
N-{[1-butyl-4-chloro-2-(2-methylphenyl)-1H-imidazol-5-yl]methyl}-N-(quinoxalin-6-ylmethyl)butan-1-amine;
5-(5-{butyl[(1-butyl-4-chloro-2-phenyl-1H-imidazol-5-yl)methyl]amino}pentyl)isoxazol-3-ol;
methyl [[(1-butyl-4-chloro-2-phenyl-1H-imidazol-5-yl)methyl](2,3-dihydro-1,4-benzodioxin-6-ylmethyl)amino](phenyl)acetate;
N-[(1-butyl-2-phenyl-4-vinyl-1H-imidazol-5-yl)methyl]-N-(2,3-dihydro-1,4-benzodioxin-6-ylmethyl)-N-(3-ethoxybenzyl)amine;
methyl 4-{[[(1-butyl-2-phenyl-4-vinyl-1H-imidazol-5-yl)methyl](2,3-dihydro-1,4-benzodioxin-6-ylmethyl)amino]methyl}benzoate;
N-[(1-butyl-4-phenyl-2-vinyl-1H-imidazol-5-yl)methyl]-N-(2,3-dihydro-1,4-benzodioxin-6-ylmethyl)-N-(3-ethoxybenzyl)amine;
N-[(1-butyl-4-ethyl-2-phenyl-1H-imidazol-5-yl)methyl]-N-(2,3-dihydro-1,4-benzodioxin-6-ylmethyl)-N-(3-ethoxybenzyl)amine;
methyl 4-{[[(1-butyl-4-ethyl-2-phenyl-1H-imidazol-5-yl)methyl](2,3-dihydro-1,4-benzodioxin-6-ylmethyl)amino]methyl}benzoate;
(1S)-N-(1,3-benzodioxol-5-ylmethyl)-1-(1-butyl-2,4-diphenyl-1H-imidazol-5-yl)-N-methylpentan-1-amine;
N-[(1-butyl-4-chloro-2-phenyl-1H-imidazol-5-yl)methyl]-N-(2,3-dihydro-1,4-benzodioxin-6-ylmethyl)-N-(quinoxalin-6-ylmethyl)amine;
4-{[[(1-butyl-2-phenyl-4-vinyl-1H-imidazol-5-yl)methyl](2,3-dihydro-1,4-benzodioxin-6-ylmethyl)amino]methyl}benzoic acid;
N-{1-[1-butyl-4-chloro-2-(2-methylphenyl)-1H-imidazol-5-yl]ethyl}-N-(2,3-dihydro-1,4-benzodioxin-6-ylmethyl)butan-1-amine;
N-{1-[1-butyl-4-chloro-2-(2-methylphenyl)-1H-imidazol-5-yl]ethyl}-N-(2,3-dihydro-1,4-benzodioxin-6-ylmethyl)-3-methylbutan-1-amine;
N-{1-[1-butyl-4-chloro-2-(2-methylphenyl)-1H-imidazol-5-yl]ethyl}-N-(1H-indol-5-ylmethyl)butan-1-amine;
N-{[1-butyl-4-chloro-2-(2-methylphenyl)-1H-imidazol-5-yl]methyl}-3-methyl-N-[(2-methyl-1H-indol-5-yl)methyl]butan-1-amine;
1-(1-butyl-2-phenyl-1H-imidazol-5-yl)-N-(cyclohexylmethyl)-N-methylpentan-1-amine;
4-{[[(1-butyl-4-ethyl-2-phenyl-1H-imidazol-5-y])methyl](2,3-dihydro-1,4-benzodioxin-6-ylmethyl)amino]methyl}benzoic acid;
N-{[4-chloro-1-(ethoxymethyl)-2-phenyl-1H-imidazol-5-yl]methyl}-N-(2,3-dihydro-1,4-benzodioxin-6-ylmethyl)-N-(3-ethoxybenzyl)amine;
1-(4-chloro-2-phenyl-1H-imidazol-5-yl)-N-(2,3-dihydro-1,4-benzodioxin-6-ylmethyl)-N-(3-ethoxybenzyl)methanamine;
N-{[1-butyl-4-chloro-2-(2-methylphenyl)-1H-imidazol-5-yl]methyl}-N-methyl-1,2,3,4-tetrahydronaphthalen-1-amine;
1-(1-butyl-2,4-diphenyl-1H-imidazol-5-yl)-N-(cyclohexylmethyl)-N-(1,2,3,4-tetrahydroquinolin-6-ylmethyl)methanamine;
1-[1-butyl-2-phenyl-4-(trifluoromethyl)-1H-imidazol-5-yl]-N-(cyclohexylmethyl)-N-(1,2,3,4-tetrahydroquinolin-6-ylmethyl)methanamine;
N-{[4-bromo-1-butyl-2-(2,6-diethylphenyl)-1H-imidazol-5-yl]methyl}-N-methyl-1,2,3,4-tetrahydronaphthalen-1-amine;
methyl 4-{[{[1-butyl-4-(4-methylphenyl)-2-phenyl-1H-imidazol-5-yl]methyl}(2,3-dihydro-1,4-benzodioxin-6-ylmethyl)amino]methyl}benzoate;
methyl 4-{[{[1-butyl-4-(3-fluorophenyl)-2-phenyl-1H-imidazol-5-yl]methyl}(2,3-dihydro-1,4-benzodioxin-6-ylmethyl)amino]methyl benzoate;
4-{[[[1-butyl-4-(4-methylphenyl)-2-phenyl-1H-imidazol-5-yl]methyl}(2,3-dihydro-1,4-benzodioxin-6-ylmethyl)amino]methyl}benzoic acid;
4-{[{[1-butyl-4-(3-fluorophenyl)-2-phenyl-1H-imidazol-5-yl]methyl}(2,3-dihydro-1,4-benzodioxin-6-ylmethyl)amino]methyl}benzoic acid;
N-{[1-butyl-4-chloro-2-(2-methylphenyl)-1H-imidazol-5-yl]methyl}-N-(3-methoxybenzyl)butan-1-amine;
N-{[1-butyl-4-chloro-2-(2-methylphenyl)-1H-imidazol-5-yl]methyl}-N-(3-ethoxybenzyl)butan-1-amine;
N-{[1-butyl-4-chloro-2-(2-methylphenyl)-1H-imidazol-5-yl]methyl}-N-(1H-indol-5-ylmethyl)-3,3-dimethylbutan-1-amine;
6-[(butyl {[1-butyl-4-chloro-2-(2-methylphenyl)-1H-imidazol-5-yl]methyl}amino)methyl]-3,4-dihydroquinolin-2(1H)-one; and
N-{[1-butyl-4-chloro-2-(2-methylphenyl)-1H-imidazol-5-yl]methyl}-N-(1,2,3,4-tetrahydroquinolin-6-ylmethyl)butan-1-amine.

25. A pharmaceutical composition comprising a therapeutically effective amount of a C5a antagonist, a therapeutically effective amount of a C5a receptor-inactive therapeutic agent and a physiologically acceptable carrier, wherein the C5a antagonist is a compound of the formula: 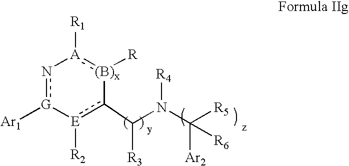
or a pharmaceutically acceptable salts thereof, wherein 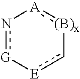
is: i) a 5-membered heteroaryl ring system, in which x is 0; A is carbon, nitrogen, oxygen, or sulfur; and E and G are independently carbon or nitrogen, provided that the 5-membered heteroaryl ring system does not contain more than 3 heteroatoms or more than 1 oxygen or sulfur atom; or
ii) a 6-membered heteroaryl ring system, in which x is 1; A, B, E, and G are independently chosen from carbon and nitrogen, provided that the 6-membered heteroaryl ring system does not contain more than 3 nitrogen atoms.
R and R₁ independently represent:
i) hydrogen, hydroxy, halogen, amino, cyano, nitro, —CHO, —CONH₂, C₁-C₆haloalkyl, or C₁-C₆ haloalkoxy;
ii) C₁-C₆alkyl, C₁-C₆alkenyl, C₁-C₆alkynyl, C₁-C₆ alkoxy, C₃-C₇cycloalkyl, (C₃-C₇cycloalkyl)C₁-C₄alkyl, mono- or di-C₁-C₆alkylamino, mono- or di-C₁-C₆alkylaminoC₁-C₆alkyl, mono- or di-C₁-C₆alkylcarboxamide, C₁-C₆alkoxycarbonyl, —NHSOₙC₁-C₆alkyl, —SOₙN(C₁-C₆alkyl) (C₁-C₆alkyl), or phenyl-SOₙ—, each of which is optionally substituted, wherein n is 0, 1, or 2; or
iii) naphthyl, phenyl, phenylC₁-C₄carbhydryl, 5- or 6-membered heteroaryl, or 5- or 6-membered heteroarylC₁-C₄carbhydryl, each of which is optionally substituted;
If E is nitrogen, R₂ is chosen from C₁-C₇alkyl, C₂-C₇alkenyl, C₂-C₇ alkynyl, C₃-C₇cycloalkyl(C₁-C₄alkyl), benzyl and C₁-C₆haloalkyl;
If E is carbon, R₂ is chosen from halogen, hydroxy, C₁-C₇alkyl, C₂-C₇alkenyl, C₂-C₇alkynyl, C₁-C₇alkoxy, C₁-C₇alkylamino, C₃-C₇cycloalkyl(C₁-C₄alkyl), benzyl, C₁-C₆haloalkyl, and C₁-C₆haloalkoxy;
R₃ is hydrogen, C₁-C₆alkyl, C₂-C₆alkenyl, hydroxyC₁-C₆alkyl, C₁-C₆haloalkyl, C₃-C₇cycloalkyl, (C₃-C₇cycloalkyl)C₁-C₄alkyl, or phenyl(C₁-C₄alkyl); or when x is 0, R₁ and R₃ may be joined to form an optionally substituted (C₃-C₇)cycloalkyl;
R₄ is i) hydrogen;
ii) C₁-C₆alkyl, C₂-C₆alkenyl, C₂-C₆alkynyl, C₃-C₇cycloalkyl, C₃-C₇cycloalkenyl, (C₃-C₇cycloalkyl)C₁-C₄alkyl, (C₃-C₇cycloalkenyl)C₁-C₄alkyl, or hexahydro-1,3-benzodioxolylmethyl, each of which is optionally substituted;
iii) optionally substituted arylC₁-C₄alkyl having 1 or 2 fused or pendant rings;
iv) optionally substituted arylC₁-C₄alkyl, wherein the aryl portion is fused to a 5- to 7-membered saturated or partially unsaturated ring having 0, 1, or 2 ring atoms independently chosen from N, O, and S with remaining ring atoms being carbon;
v) optionally substituted heterocycloalkyl(C₀-C₄alkyl);
vi) optionally substituted heteroarylC₁-C₂alkyl having from 1 to 2 fused or pendant rings, from 5 to 7 members in each ring, and in at least one ring from 1 to 3 heteroatoms independently selected from N, O, and S; or
vii) optionally substituted saturated or partially unsaturated heterocyclic(C₀-C₄alkyl) having from 4 to 7 ring members, 1 or 2 of which ring members are N, S or O, with remaining ring members being carbon;
R₅ and R₆ are independently chosen from hydrogen and C₁-C₆alkyl;
z is 1,2, or 3;
Ar₁ is i) optionally substituted aryl having 1 or 2 fused or pendant rings;
ii) optionally substituted phenyl fused to a 5- to 7-membered saturated or partially unsaturated ring having 0, 1, or 2 ring atoms independently chosen from N, O, and S with remaining ring atoms being carbon; or
iii) optionally substituted heteroaryl having 1 or 2 fused or pendant rings, from 5 to 7 members in each ring, and in at least one ring from 1 to 3 heteroatoms independently selected from N, O, and S;
Ar₂ is i) optionally substituted C₃-C₇cycloalkyl, C₃-C₇cycloalkyl(C₁-C₄alkyl), C₃-C₇cycloalkenyl, C₃-C₇cycloalkenyl(C₁-C₄alkyl), or hexahydro-1,3-benzodioxolyl;
ii) optionally substituted aryl having 1 or 2 fused or pendant rings;
iii) optionally substituted phenyl fused to a 5- to 7-membered saturated or partially unsaturated ring having 0, 1, or 2 ring atoms independently chosen from N, O, and S with remaining ring atoms being carbon; or
iv) optionally substituted heteroaryl having 1 or 2 fused or pendant rings, from 5 to 7 members in each ring, and in at least one ring from 1 to 3 heteroatoms independently selected from N, O, and S; and
y is 1 or 2.

26. A pharmaceutical composition according to claim 25, wherein the pharmaceutical composition is an injectible solution, a tablet, a capsule, or a pill.

27. A pharmaceutical composition according to claim 25, wherein the pharmaceutical composition is an inhalable preparation.

28. A pharmaceutical composition according to claim 25, wherein the pharmaceutical composition is a topical preparation.

29. A package comprising a pharmaceutical composition according to claim 25 and instructions for using the pharmaceutical composition to treat a patient suffering from a condition with a pathogenic inflammatory component.

30. A package according to claim 29, wherein the condition is an autoimmune disorder, asthma, cardio- or cerebrovascular disease, psoriasis, reperfusion injury, burn, traumatic CNS or spinal cord injury, rheumatoid arthritis, psoriasis, asthma, or cardio or cerebrovascular disease.

31. A package comprising a pharmaceutical composition according to claim 25 and instructions for using the pharmaceutical composition to reduce the risk of myocardial infarction or stroke in a patient at risk for myocardial infarction or stroke.

32. A method for treating a condition with a pathogenic inflammatory component in a patient, comprising administering to a patient a therapeutically effective amount of a C5a receptor antagonist and a therapeutically effective amount of a C5a receptor-inactive therapeutic agent, wherein the C5a antagonist is a compound of the formula: 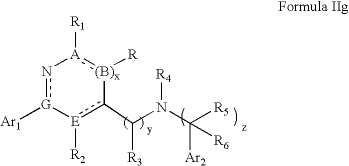
or a pharmaceutically acceptable salts thereof, wherein 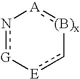
is: i) a 5-membered heteroaryl ring system, in which x is 0; A is carbon, nitrogen, oxygen, or sulfur; and E and G are independently carbon or nitrogen, provided that the 5-membered heteroaryl ring system does not contain more than 3 heteroatoms or more than 1 oxygen or sulfur atom; or
ii) a 6-membered heteroaryl ring system, in which x is 1; A, B, E, and G are independently chosen from carbon and nitrogen, provided that the 6-membered heteroaryl ring system does not contain more than 3 nitrogen atoms.
R and R₁ independently represent:
i) hydrogen, hydroxy, halogen, amino, cyano, nitro, —CHO, —CONH₂, C₁-C₆haloalkyl, or C₁-C₆ haloalkoxy;
ii) C₁-C₆alkyl, C₁-C₆alkenyl, C₁-C₆alkynyl, C₁-C₆ alkoxy, C₃-C₇cycloalkyl, (C₃-C₇cycloalkyl)C₁-C₄alkyl, mono- or di-C₁-C₆alkylamino, mono- or di-C₁-C₆alkylaminoC₁-C₆alkyl, mono- or di-C₁-C₆alkylcarboxamide, C₁-C₆alkoxycarbonyl, —NHSOₙC₁-C₆alkyl, —SOₙN(C₁-C₆alkyl) (C₁-C₆alkyl), or phenyl-SOₙ—, each of which is optionally substituted, wherein n is 0, 1 or 2; or
iii) naphthyl, phenyl, phenylC₁-C₄carbhydryl, 5- or 6-membered heteroaryl, or 5- or 6-membered heteroarylC₁-C₄carbhydryl, each of which is optionally substituted;
If E is nitrogen, R₂ is chosen from C₁-C₇alkyl, C₂-C₇alkenyl, C₂-C₇ alkynyl, C₃-C₇Cycloalkyl(C₁-C₄alkyl), benzyl and C₁-C₆haloalkyl;
If E is carbon, R₂ is chosen from halogen, hydroxy, C₁-C₇alkyl, C₂-C₇alkenyl, C₂-C₇alkynyl, C₁-C₇alkoxy, C₁-C₇alkylamino, C₃-C₇cycloalkyl(C₁-C₄alkyl), benzyl, C₁-C₆haloalkyl, and C₁-C₆haloalkoxy;
R₃ is hydrogen, C₁-C₆alkyl, C₂-C₆alkenyl, hydroxyC₁-C₆alkyl, C₁-C₆haloalkyl, C₃-C₇cycloalkyl, (C₃-C₇cycloalkyl)C₁-C₄alkyl, or phenyl(C₁-C₄alkyl); or when x is 0, R₁ and R₃ may be joined to form an optionally substituted (C₃-C₇)cycloalkyl;
R₄ is i) hydrogen;
ii) C₁-C₆alkyl, C₂-C₆alkenyl, C₂-C₆alkynyl, C₃-C₇cycloalkyl, C₃-C₇cycloalkenyl, (C₃-C₇Cycloalkyl)C₁-C₄alkyl, (C₃-C₇cycloalkenyl)C₁-C₄alkyl, or hexahydro-1,3-benzodioxolylmethyl, each of which is optionally substituted;
iii) optionally substituted arylC₁-C₄alkyl having 1 or 2 fused or pendant rings;
iv) optionally substituted arylC₁-C₄alkyl, wherein the aryl portion is fused to a 5- to 7-membered saturated or partially unsaturated ring having 0, 1, or 2 ring atoms independently chosen from N, O, and S with remaining ring atoms being carbon;
v) optionally substituted heterocycloalkyl(C₀C₄alkyl);
vi) optionally substituted heteroarylC₁-C₂alkyl having from 1 to 2 fused or pendant rings, from 5 to 7 members in each ring, and in at least one ring from 1 to 3 heteroatoms independently selected from N, O, and S; or
vii) optionally substituted saturated or partially unsaturated heterocyclic(Co-C₄alkyl) having from 4 to 7 ring members, 1 or 2 of which ring members are N, S or O, with remaining ring members being carbon;
R₅ and R₆ are independently chosen from hydrogen and C₁-C₆alkyl;
z is 1, 2, or 3;
Ar₁ is i) optionally substituted aryl having 1 or 2 fused or pendant rings;
ii) optionally substituted phenyl fused to a 5- to 7-membered saturated or partially unsaturated ring having 0, 1, or 2 ring atoms independently chosen from N, O, and S with remaining ring atoms being carbon; or
iii) optionally substituted heteroaryl having 1 or 2 fused or pendant rings, from 5 to 7 members in each ring, and in at least one ring from 1 to 3 heteroatoms independently selected from N, O, and S;
Ar₂ is i) optionally substituted C₃-C₇cycloalkyl, C₃-C₇cycloalkyl(C₁-C₄alkyl), C₃-C₇cycloalkenyl, C₃-C₇cycloalkenyl(C₁-C₄alkyl), or hexahydro-1,3-benzodioxolyl;
ii) optionally substituted aryl having 1 or 2 fused or pendant rings;
iii) optionally substituted phenyl fused to a 5- to 7-membered saturated or partially unsaturated ring having 0, 1, or 2 ring atoms independently chosen from N, O, and S with remaining ring atoms being carbon; or
iv) optionally substituted heteroaryl having 1 or 2 fused or pendant rings, from 5 to 7 members in each ring, and in at least one ring from 1 to 3 heteroatoms independently selected from N, O, and S; and
y is 1 or 2.

33. A method according to claim 32, wherein the disorder is an autoimmune disorder, asthma, cardio- or cerebrovascular disease, psoriasis, reperfusion injury, burn, traumatic CNS or spinal cord injury, rheumatoid arthritis, asthma, psoriasis, or cardio- or cerebrovascular disease.

34. A method according to claim 32, wherein the patient is a human.

35. A process for preparing a pharmaceutical composition, which comprises combining a therapeutically effective amount of a C5a antagonist, a therapeutically effective amount of a therapeutic agent that is not a C5a antagonist, and a pharmaceutically acceptable carrier, wherein the C5a antagonist is a compound of the formula: 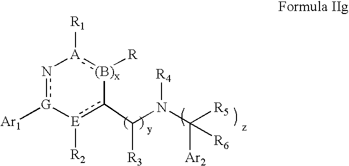
or a pharmaceutically acceptable salts thereof, wherein 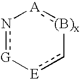
is: i) a 5-membered heteroaryl ring system, in which x is 0; A is carbon, nitrogen, oxygen, or sulfur; and E and G are independently carbon or nitrogen, provided that the 5-membered heteroaryl ring system does not contain more than 3 heteroatoms or more than 1 oxygen or sulfur atom; or
ii) a 6-membered heteroaryl ring system, in which x is 1; A, B, E, and G are independently chosen from carbon and nitrogen, provided that the 6-membered heteroaryl ring system does not contain more than 3 nitrogen atoms.
R and R₁ independently represent:
i) hydrogen, hydroxy, halogen, amino, cyano, nitro, —CHO, —CONH₂, C₁-C₆haloalkyl, or C₁-C₆ haloalkoxy;
ii) C₁-C₆alkyl, C₁-C₆alkenyl, C₁-C₆alkynyl, C₁-C₆ alkoxy, C₃-C₇cycloalkyl, (C₃-C₇cycloalkyl)C₁-C₄alkyl, mono- or di-C₁-C₆alkylamino, mono- or di-C₁-C₆alkylaminoC₁-C₆alkyl, mono- or di-C₁-C₆alkylcarboxamide, C₁-C₆alkoxycarbonyl, —NHSOₙC₁-C₆alkyl, —SOₙN(C₁-C₆alkyl) (C₁-C₆alkyl), or phenyl-SOₙ—, each of which is optionally substituted, wherein n is 0, 1 or 2; or
iii) naphthyl, phenyl, phenylC₁-C₄carbhydryl, 5- or 6-membered heteroaryl, or 5- or 6-membered heteroary]C₁-C₄carbhydryl, each of which is optionally substituted;
If E is nitrogen, R₂ is chosen from C₁-C₇alkyl, C₂-C₇alkenyl, C₂-C₇ alkynyl, C₃-C₇cycloalkyl(C₁-C₄alkyl), benzyl and C₁-C₆haloalkyl;
If E is carbon, R₂ is chosen from halogen, hydroxy, C₁-C₇alkyl, C₂-C₇alkenyl, C₂-C₇alkynyl, C₁-C₇alkoxy, C₁-C₇alkylamino, C₃-C₇cycloalkyl(C₁-C₄alkyl), benzyl, C₁-C₆haloalkyl, and C₁-C₆haloalkoxy;
R₃ is hydrogen, C₁-C₆alkyl, C₂-C₆alkenyl, hydroxyC₁-C₆alkyl, C₁-C₆haloalkyl, C₃-C₇cycloalkyl, (C₃-C₇cycloalkyl)C₁-C₄alkyl, or phenyl(C₁-C₄alkyl); or when x is 0, R₁ and R₃ may be joined to form an optionally substituted (C₃-C₇)cycloalkyl;
R₄ is i) hydrogen;
ii) C₁-C₆alkyl, C₂-C₆alkenyl, C₂-C₆alkynyl, C₃-C₇cycloalkyl, C₃-C₇Cycloalkenyl, (C₃-C₇cycloalkyl)C₁-C₄alkyl, (C₃-C₇cycloalkenyl)C₁-C₄alkyl, or hexahydro-1,3-benzodioxolylmethyl, each of which is optionally substituted;
iii) optionally substituted arylC₁-C₄alkyl having 1 or 2 fused or pendant rings;
iv) optionally substituted arylC₁-C₄alkyl, wherein the aryl portion is fused to a 5- to 7-membered saturated or partially unsaturated ring having 0, 1, or 2 ring atoms independently chosen from N, O, and S with remaining ring atoms being carbon;
v) optionally substituted heterocycloalkyl(C₀C₄alkyl);
vi) optionally substituted heteroarylC₁-C₂alkyl having from 1 to 2 fused or pendant rings, from 5 to 7 members in each ring, and in at least one ring from 1 to 3 heteroatoms independently selected from N, O, and S; or
vii) optionally substituted saturated or partially unsaturated heterocyclic(C₀C₄alkyl) having from 4 to 7 ring members, 1 or 2 of which ring members are N, S or O, with remaining ring members being carbon;
R₅ and R₆ are independently chosen from hydrogen and C₁-C₆alkyl;
z is 1, 2, or 3;
Ar₁ is i) optionally substituted aryl having 1 or 2 fused or pendant rings;
ii) optionally substituted phenyl fused to a 5- to 7-membered saturated or partially unsaturated ring having 0, 1, or 2 ring atoms independently chosen from N, O, and S with remaining ring atoms being carbon; or
iii) optionally substituted heteroaryl having 1 or 2 fused or pendant rings, from 5 to 7 members in each ring, and in at least one ring from 1 to 3 heteroatoms independently selected from N, O, and S;
Ar₂ is i) optionally substituted C₃-C₇cycloalkyl, C₃-C₇cycloalkyl(C₁-C₄alkyl), C₃-C₇cycloalkenyl, C₃-C₇cycloalkenyl(C₁-C₄alkyl), or hexahydro-1,3-benzodioxolyl;
ii) optionally substituted aryl having 1 or 2 fused or pendant rings;
iii) optionally substituted phenyl fused to a 5- to 7-membered saturated or partially unsaturated ring having 0, 1, or 2 ring atoms independently chosen from N, O, and S with remaining ring atoms being carbon; or
iv) optionally substituted heteroaryl having 1 or 2 fused or pendant rings, from 5 to 7 members in each ring, and in at least one ring from 1 to 3 heteroatoms independently selected from N, O, and S; and
y is 1 or 2.
